(19) United States
(12) Patent Application Publication
(10) Pub. No.: US 2026/0258007 A1
(43) Pub. Date: Sep. 3, 2026

(54) COMPOUNDS, COMPOSITIONS AND METHODS OF USE TO TREAT HYPOPARATHYROIDISM AND OSTEOPOROSIS

(71) Applicant: SEPTERNA, INC., South San Francisco, CA (US)

(72) Inventors: Xiaohui Du, Belmont, CA (US); Xiang Zhou, Hayward, CA (US); Todd J.A. Ewing, Walnut Creek, CA (US); Daniel D. Long, San Francisco, CA (US); Steven j. McKerrall, San Francisco, CA (US); Paul Gibbons, San Francisco, CA (US); Matthew Volgraf, Oakland, CA (US); Robin Larouche-Gauthier, Vaudreuil -Dorion (CA); Samir Bouayad-Gervais, Montreal (CA); Curtis Eugene Colwell, Orleans (CA); Vincent Albert, Rigaud (CA); Liang Zhao, Foster City, CA (US)

(21) Appl. No.: 19/148,193

(22) PCT Filed: Jan. 16, 2024
(86) PCT No.: PCT/US2024/011633

§ 371 Date: Jul. 15, 2025

Related U.S. Application Data
(60) Provisional application No. 63/439,471, filed on Jan. 17, 2023.
(60) Provisional application No. 63/471,847, filed on Jun. 8, 2023.

(51) Int. Cl. C07D 277/82; A61K 31/405; A61K 31/417; A61K 31/4184; A61K 31/423; A61K 31/428; A61K 31/433; A61K 31/437; A61K 31/4439; A61K 31/47; A61K 31/505; A61K 31/506; A61K 31/517; C07D 209/24; C07D 209/40; C07D 215/38; C07D 233/90; C07D 235/30; C07D 239/48; C07D 263/58; C07D 417/04; C07D 417/06; C07D 417/12; C07D 417/14; C07D 471/04; C07D 487/04; C07D 513/04

(52) U.S. Cl. CPC C07D 277/82; A61K 31/405; A61K 31/417; A61K 31/4184; A61K 31/423; A61K 31/428; A61K 31/433; A61K 31/437; A61K 31/4439; A61K 31/47; A61K 31/505; A61K 31/506; A61K 31/517; C07D 209/24; C07D 209/40; C07D 215/38; C07D 233/90; C07D 235/30; C07D 239/48; C07D 263/58; C07D 417/04; C07D 417/06; C07D 417/12; C07D 417/14; C07D 471/04; C07D 487/04; C07D 513/04

(57) ABSTRACT

Disclosed are compounds that are parathyroid hormone receptor 1 agonists, and methods of using them for preventing or treating osteoporosis, fracture, osteomalacia, arthritis, thrombocytopenia, hypoparathyroidism, hyperphosphatemia or tumoral calcinosis.

### CROSS-REFERENCE TO RELATED APPLICATIONS

This application claims the benefit of priority to U.S. Provisional Application Nos. 63/471,847, filed Jun. 8, 2023; and 63/439,471, filed Jan. 17, 2023; each of which is incorporated herein by reference in ts entirety.

### BACKGROUND

Regulation of calcium concentration is important to normal function of the gastrointestinal tract, skeletal system, nervous system, muscular nervous system and cardiovascular system. Parathyroid hormone (PTH) synthesis and release is primarily controlled by serum calcium levels.

Osteoporosis is characterized by bone loss resulting in an increased incidence of fracture. This condition, which is most prevalent in the spine and hip, affects 1 in 3 postmenopausal women, a lesser but significant number of aging men, and is also caused by other conditions including hypogonadism and prolonged glucocorticoid use. Current therapies to treat osteoporosis, such as bisphosphonates, hormone replacement therapy, SERMs and calcitonin, serve to arrest further bone loss by inhibiting bone resorption. Although these treatments may slow or even prevent continued bone loss, new bone formation leading to increased bone mass and strength, does not occur. Consequently, there is still a need for a therapeutic agent capable of stimulating bone formation. Such a therapeutic agent would be beneficial both to patients who are at risk of developing osteoporosis or who present with established osteoporosis.

Parathyroid hormone (PTH) is a significant regulator of calcium homeostasis and acts, in part, by mobilizing calcium from the skeleton through increased bone resorption. Additionally, pulsatile administration of PTH can stimulate new bone formation, both in laboratory animals and in humans. Thus, there is evidence to suggest that targeting of the receptor for PTH with a small molecule agonist mimicking the actions of PTH, would be a suitable approach for generating an anabolic response in bone. PTH elicits its effects by binding and activating a class B, G protein-coupled receptor of the 7 transmembrane superfamily, designated PTH1R. PTH1R activates multiple signaling pathways, but predominantly the adenylyl cyclase/cyclic AMP and the phospholipase C/calcium mobilization pathways.

Accordingly, there is a need in the art to provide small molecule therapeutics that treat or prevent hypoparathyroidism, osteoporosis and related conditions. In particular, there is a need for providing compounds that act as PTH1R agonists.

### SUMMARY

One aspect of the invention provides compounds, compositions, and methods useful for preventing or treating or preventing osteoporosis, fracture, osteomalacia, arthritis, thrombocytopenia, hypoparathyroidism, hyperphosphatemia or tumoral calcinosis.

Accordingly, provided herein in some embodiments is a compound having the structure of Formula (I):

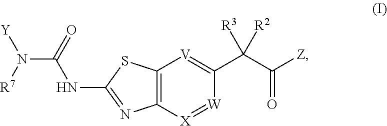

or a pharmaceutically acceptable salt thereof;
wherein:
   Q is O or S;
   V, W, and X are independently N or CR¹; provided that at least one of V, W, and X is CR¹;
   Y is (C₁-C₆)alkyl, (C₁-C₆)cycloalkyl, 4- to 7-membered heterocycloalkyl, phenyl, or 5- or 6-membered heteroaryl; each of which is optionally substituted with one, two, or three substituents selected independently for each occurrence from fluoro, chloro, cyano, hydroxyl, (C₁-C₆)alkyl, (C₃-C₈)cycloalkyl, (C₁-C₆)haloalkyl, (C₁-C₆)hydroxyalkyl, (C₁-C₆)alkoxyalkyl, and phenyl; each of which substituents is optionally substituted with one, two, or three substituents selected independently for each occurrence from fluoro, (C₁-C₆)alkyl, (C₁-C₆)haloalkyl, (C₁-C₆)hydroxyalkyl, and (C₁-C₆)alkoxyalkyl;
   Z is NR⁴R⁵ or OR⁶;
   R¹ is independently for each occurrence H, (C₁-C₆)alkyl, (C₁-C₆)haloalkyl, (C₁-C₆)hydroxyalkyl, (C₁-C₆)alkoxyalkyl, fluoro, chloro, cyano, hydroxyl, or (C₁-C₆)alkoxy;
   R² is (C₁-C₆)haloalkyl, (C₁-C₆)alkyl, or (C₁-C₆)cycloalkyl;
   R³ is hydroxyl, (C₁-C₆)alkyl, (C₁-C₆)alkoxyalkyl, (C₁-C₆)hydroxyalkyl, NR¹¹R¹², NC(O)R¹³, OC(O)NR¹¹R¹², or NR¹⁴SO₂R¹⁵; or R² and R³ together with the carbon atom to which they are attached form a (C₃-C₈)cycloalkyl or a 4- to 7-membered heterocycloalkyl; wherein (C₃-C₈)cycloalkyl or 4- to 7-membered heterocycloalkyl is optionally substituted with one, two, or three substituents selected independently for each occurrence from fluoro, chloro, cyano, hydroxyl, (C₁-C₆)alkyl, (C₁-C₆)haloalkyl, (C₁-C₆)hydroxyalkyl, and (C₁-C₆)alkoxyalkyl;
   R⁴ is hydrogen, (C₁-C₆)alkyl, (C₃-C₈)cycloalkyl, 4- to 7-membered heterocycloalkyl, phenyl, or 5- or 6-membered heteroaryl; wherein (C₁-C₆)alkyl is optionally substituted with one, two or three substituents selected independently for each occurrence from R^{4a}, halo, hydroxyl, cyano, carboxy, (C₁-C₆)alkylsulfonyl, (C₁-C₆)alkylcarbonyl, C(O)NR¹¹R¹², NR¹¹R¹², OC(O)NR¹¹R¹² and (C₁-C₆)alkoxy; and wherein (C₃-C₈)cycloalkyl, 4- to 7-membered heterocycloalkyl, phenyl, or 5- or 6-membered heteroaryl is optionally substituted with one, two, or three substituents selected independently for each occurrence from fluoro, chloro, cyano, hydroxyl, 5- to 10-membered heteroaryl, (C₁-C₆)alkyl, (C₁-C₆)haloalkyl, (C₁-C₆)hydroxyalkyl, (C₁-C₆)alkoxyalkyl, (C₁-C₆)alkylsulfonyl, (C₁-C₆)alkylcarbonyl C(O)NR¹¹R¹², NR¹¹R¹², NC(O)OR¹³, NC(O)R¹³, OC(O)NR¹¹R¹², and SO₂NR¹¹R¹²;
   R^{4a} is (C₃-C₈)cycloalkyl, phenyl, 5- to 10-membered heteroaryl, or 4- to 7-membered heterocycloalkyl, each of which is optionally substituted with one, two, or three substituents selected independently for each occurrence from fluoro, chloro, cyano, hydroxyl, (C₁-C₆)alkyl, (C₁-C₆)haloalkyl, (C₁-C₆)hydroxyalkyl, (C₁-C₆)alkoxyalkyl, and (C₁-C₆)alkylsulfonyl;
   R⁵ is hydrogen or (C₁-C₆)alkyl; or R⁴ and R⁵ taken together with the nitrogen atom to which they are attached form an optionally 4- to 7-membered heterocycloalkyl; wherein 4- to 7-membered heterocycloalkyl is optionally substituted with one, two, or three substituents selected independently for each occurrence from fluoro, chloro, cyano, hydroxyl, carboxyl, 5- to 10-membered heteroaryl, (C₁-C₆)alkyl, (C₁-C₆)haloalkyl, (C₁-C₆)hydroxyalkyl, (C₁-C₆)alkoxyalkyl, (C₁-C₆)carboxyalkyl, (C₁-C₆)alkylsulfonyl, NR¹⁴SO₂R¹⁵, and C(O)NR¹¹R¹²;
   R⁶ is hydrogen or (C₁-C₆)alkyl;
   R⁷ is hydrogen or (C₁-C₆)alkyl; or R⁷ and Y taken together with the nitrogen atom to which they are attached form a 4- to 7-membered heterocycloalkyl:
   R¹¹ and R¹² are independently for each occurrence selected from hydrogen, (C₁-C₆)alkyl, and (C₃-C₈)cycloalkyl; or R¹¹ and R¹² taken together with the atoms to which they are attached form a 4- to 7-membered heterocycloalkyl;
   R¹³ is independently for each occurrence selected from (C₁-C₆)alkyl and (C₃-C₈)cycloalkyl; and
   R¹⁴ is independently for each occurrence selected from hydrogen, (C₁-C₆)alkyl, and (C₃-C₈)cycloalkyl, and R¹⁵ is independently for each occurrence selected from (C₁-C₆)alkyl and (C₁-C₆)cycloalkyl; or R¹⁴ and R¹⁵ taken together with the atoms to which they are attached form a 4- to 7-membered heterocycloalkyl.

Provided in other embodiments is a compound having the structure of Formula (Ia):

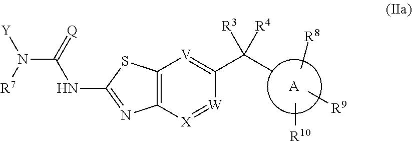

or a pharmaceutically acceptable salt thereof;
wherein:
   A is 5- to 10-membered heteroaryl;
   Q is S or O;
   V, W, and X are independently N or CR¹; provided that at least one of V, W, and X is CR¹;
   Y is (C₁-C₆)alkyl, (C₁-C₆)cycloalkyl, 4- to 7-membered heterocycloalkyl, phenyl, or 5- or 6-membered heteroaryl; each of which is optionally substituted with one, two, or three substituents selected independently for each occurrence from fluoro, chloro, cyano, hydroxyl, (C₁-C₆)alkyl, (C₁-C₆)cycloalkyl, (C₁-C₆)haloalkyl, (C₁-C₆)hydroxyalkyl, (C₁-C₆)alkoxyalkyl, and phenyl; each of which substituents is optionally substituted with one, two, or three substituents selected independently for each occurrence from fluoro, (C₁-C₆)alkyl, (C₁-C₆)haloalkyl, (C₁-C₆)hydroxyalkyl, and (C₁-C₆)alkoxyalkyl;
   R¹ is independently for each occurrence H, (C₁-C₆)alkyl, (C₁-C₆)haloalkyl, (C₁-C₆)hydroxyalkyl, (C₁-C₆)alkoxyalkyl, fluoro, chloro, cyano, hydroxyl, or (C₁-C₆)alkoxy;
   R² is (C₁-C₆)haloalkyl, (C₁-C₆)alkyl, or (C₃-C₈)cycloalkyl;
   R³ is hydroxyl, (C₁-C₆)alkyl, (C₁-C₆)alkoxyalkyl, (C₁-C₆)hydroxyalkyl, NR¹¹R¹², NC(O)R¹³, OC(O)NR¹¹R¹², NR¹¹SO₂R¹²; or R² and R³ together with the carbon atom to which they are attached form a (C₁-C₆)cycloalkyl or a 4- to 7-membered heterocycloalkyl; wherein (C₃-C₈)cycloalkyl or 4- to 7-membered heterocycloalkyl is optionally substituted with one, two, or three substituents selected independently for each occurrence from fluoro, chloro, cyano, hydroxyl, (C₁-C₆)alkyl, (C₁-C₆)haloalkyl, (C₁-C₆)hydroxyalkyl, and (C₁-C₆)alkoxyalkyl;
   R⁷ is hydrogen or (C₁-C₆)alkyl; or R⁷ and Y taken together with the nitrogen atom to which they are attached form a 4- to 7-membered heterocycloalkyl;
   R⁸, R⁹ and R¹⁰ are independently for each occurrence H, (C₁-C₆)alkyl, or carboxy; wherein each (C₁-C₆)alkyl is optionally substituted with one, two, or three substituents selected independently for each occurrence from the group consisting of (C₁-C₆)alkoxy, hydroxy, fluoro, chloro, cyano, and NR¹¹R¹²,
   R¹¹ and R¹² are independently for each occurrence selected from hydrogen. (C₁-C₆)alkyl, and (C₃-C₈)cycloalkyl; or R¹¹ and R¹² taken together with the atoms to which they are attached form a 4- to 7-membered heterocycloalkyl;
   R¹³ is independently for each occurrence selected from (C₁-C₆)alkyl and (C₃-C₈)cycloalkyl; and
   R¹⁴ is independently for each occurrence selected from hydrogen, (C₁-C₆)alkyl, and (C₃-C₈)cycloalkyl, and R¹⁵ is independently for each occurrence selected from (C₁-C₆)alkyl and (C₃-C₈)cycloalkyl; or R¹⁴ and R¹⁵ taken together with the atoms to which they are attached form a 4- to 7-membered heterocycloalkyl.

Other aspects of the disclosure provide a pharmaceutical composition comprising a compound of formula (I), or a pharmaceutically acceptable salt thereof, and at least one pharmaceutically acceptable excipient.

In still other aspects, provided herein is a method for treating or preventing osteoporosis, fracture, osteomalacia, arthritis, thrombocytopenia, hypoparathyroidism, hyperphosphatemia or tumoral calcinosis, comprising administering to a subject in need thereof an effective amount of a compound of compound of formula ( ), or a pharmaceutically acceptable salt thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features, objects, and advantages of the invention will be apparent from the detailed description, and from the claims.

### BRIEF DESCRIPTION OF THE FIGURES

FIG. 1 tabulates exemplary compounds of the invention, and their characterization data and biological activity.

FIG. 2 tabulates additional exemplary compounds of the invention, and their characterization data and biological activity.

FIG. 3 tabulates additional exemplary compounds of the invention, and their characterization data and biological activity.

### DETAILED DESCRIPTION

### Definitions

For convenience, before further description of the present invention, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

In order for the present invention to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list. “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including.” “carrying,” “having.” “containing.” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in “atropisomeric” forms or as “atropisomers.” Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from a mixture of isomers. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Percent purity by mole fraction is the ratio of the moles of the enantiomer (or diastereomer) or over the moles of the enantiomer (or diastereomer) plus the moles of its optical isomer. When the stereochemistry of a disclosed compound is named or depicted by structure, the named or depicted stereoisomer is at least about 60%, about 70%, about 80%, about 90%, about 99% or about 99.9% by mole fraction pure relative to the other stereoisomers. When a single enantiomer is named or depicted by structure, the depicted or named enantiomer is at least about 60%, about 70%, about 80%, about 90%, about 99% or about 99.9% by mole fraction pure. When a single diastereomer is named or depicted by structure, the depicted or named diastereomer is at least about 60%, about 70%, about 80%, about 90%, about 99% or about 99.9% by mole fraction pure.

When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

Structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention.

The term “prodrug” as used herein encompasses compounds that, under physiological conditions, are converted into therapeutically active agents. A common method for making a prodrug is to include selected moieties that are hydrolyzed under physiological conditions to reveal the desired molecule. In other embodiments, the prodrug is converted by an enzymatic activity of the host animal.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body, to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth: (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphtbylate, mesylate, glucoheptonate, lactobionate, and laurylsulphonate salts, and the like. (See, for example, Berge et al. (1977) “Pharmaceutical Salts”, J. Pharm. Sci. 66:1-19.)

In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

The term “pharmaceutically acceptable cocrystals” refers to solid coformers that do not form formal ionic interactions with the small molecule.

A “therapeutically effective amount” (or “effective amount”) of a compound with respect to use in treatment, refers to an amount of the compound in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “patient” or “subject” refers to a mammal in need of a particular treatment. In certain embodiments, a patient is a primate, canine, feline, or equine. In certain embodiments, a patient is a human.

An aliphatic chain comprises the classes of alkyl, alkenyl and alkynyl defined below. A straight aliphatic chain is limited to unbranched carbon chain moieties. As used herein, the term “aliphatic group” refers to a straight chain, branched-chain, or cyclic aliphatic hydrocarbon group and includes saturated and unsaturated aliphatic groups, such as an alkyl group, an alkenyl group, or an alkynyl group.

“Alkyl” refers to a fully saturated cyclic or acyclic, branched or unbranched carbon chain moiety having the number of carbon atoms specified, or up to 30 carbon atoms if no specification is made. For example, alkyl of 1 to 8 carbon atoms refers to moieties such as methyl, ethyl, propyl, butyl, pentyl, hexyl, heptyl, and octyl, and those moieties which are positional isomers of these moieties. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. In certain embodiments, a straight chain or branched chain alkyl has 30 or fewer carbon atoms in its backbone (e.g., C₁-C₃₀ for straight chains, C₃-C₃₀ for branched chains), and more preferably 20 or fewer. Alkyl groups may be substituted or unsubstituted.

As used herein, the term “heteroalkyl” refers to an alkyl moiety as hereinbefore defined which contain one or more oxygen, sulfur, nitrogen, phosphorus, or silicon atoms in place of carbon atoms.

As used herein, the term “haloalkyl” refers to an alkyl group as hereinbefore defined substituted with at least one halogen.

As used herein, the term “hydroxyalkyl” refers to an alkyl group as hereinbefore defined substituted with at least one hydroxyl.

As used herein, the term “alkylene” refers to an alkyl group having the specified number of carbons, for example from 2 to 12 carbon atoms, that contains two points of attachment to the rest of the compound on its longest carbon chain. Non-limiting examples of alkylene groups include methylene —(CH₂)—, ethylene —(CH₂CH₂)—, n-propylene —(CH₂CH₂CH₂)—, isopropylene —(CH₂CH(CH₃))—, and the like. Alkylene groups can be cyclic or acyclic, branched or unbranched carbon chain moiety, and may be optionally substituted with one or more substituents.

“Cycloalkyl” means mono- or bicyclic or bridged or spirocyclic, or polycyclic saturated carbocyclic rings, each having from 3 to 12 carbon atoms. Preferred cycloalkyls have from 3-10 carbon atoms in their ring structure, and more preferably have 3-6 carbons in the ring structure. Cycloalkyl groups may be substituted or unsubstituted.

As used herein, the term “halocycloalkyl” refers to a cycloalkyl group as hereinbefore defined substituted with at least one halogen.

“Cyclobeteroalkyl” refers to an cycloalkyl moiety as hereinbefore defined which contain one or more oxygen, sulfur, nitrogen, phosphorus, or silicon atoms in place of carbon atoms. Preferred cycloheteroalkyls have from 4-8 carbon atoms and heteroatoms in their ring structure, and more preferably have 4-6 carbons and heteroatoms in the ring structure. Cycloheteroalkyl groups may be substituted or unsubstituted.

Unless the number of carbons is otherwise specified, “lower alkyl,” as used herein, means an alkyl group, as defined above, but having from one to ten carbons, more preferably from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. Throughout the application, preferred alkyl groups are lower alkyls. In certain embodiments, a substituent designated herein as alkyl is a lower alkyl.

“Alkenyl” refers to any cyclic or acyclic, branched or unbranched unsaturated carbon chain moiety having the number of carbon atoms specified, or up to 26 carbon atoms if no limitation on the number of carbon atoms is specified; and having one or more double bonds in the moiety. Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosoenyl, docosenyl, tricosenyl, and tetracosenyl, in their various isomeric forms, where the unsaturated bond(s) can be located anywhere in the moiety and can have either the (Z) or the (E) configuration about the double bond(s)

“Alkynyl” refers to hydrocarbyl moieties of the scope of alkenyl, but having one or more triple bonds in the moiety.

The term “aryl” as used herein includes 3- to 12-membered substituted or unsubstituted single-ring aromatic groups in which each atom of the ring is carbon (i.e., carbocyclic aryl) or where one or more atoms are heteroatoms (i.e., heteroaryl). Preferably, aryl groups include 5- to 12-membered rings, more preferably 6- to 10-membered rings The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Carboycyclic aryl groups include benzene, naphthalene, phenantbrene, phenol, aniline, and the like. Heteroaryl groups include substituted or unsubstituted aromatic 3- to 12-membered ring structures, more preferably 5- to 12-membered rings, more preferably 5- to 10-membered rings, whose ring structures include one to four heteroatoms. Heteroaryl groups include, for example, pyrrole, furan, thiophene, imidazole, oxazole, thiazole, triazole, pyrazole, pyridine, pyrazine, pyridazine and pyrimidine, and the like. Aryl and heteroaryl can be monocyclic, bicyclic, or polycyclic.

The term “halo”, “halide”, or “halogen” as used herein means halogen and includes, for example, and without being limited thereto, fluoro, chloro, bromo, iodo and the like, in both radioactive and non-radioactive forms. In a preferred embodiment, halo is selected from the group consisting of fluoro, chloro and bromo.

The terms “heterocyclyl” or “heterocyclic group” or “heterocycloalkyl” refer to 3- to 12-membered ring structures, more preferably 5- to 12-membered rings, more preferably 5- to 10-membered rings, whose ring structures include one to four heteroatoms. Heterocycles can be monocyclic, bicyclic, spirocyclic, or polycyclic. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. Heterocycloalkyl groups can be fully saturated or partially saturated. Heterocycloalkyl groups include, for example, bicyclic ring systems having either or both constituent rings saturated (e.g., 2,3-dihydroindole, 4,5,6,7-tetrahydro-benzofuran, decahydroquinoline, and the like) or partially saturated (e.g., octahydroquinoline and the like) The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, and the like.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

As used herein, the definition of each expression, e.g., alkyl, m, n, etc., when it occurs more than once in any structure, is intended to be independent of its definition elsewhere in the same structure.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

In some embodiments, a “small molecule” refers to an organic, inorganic, or organometallic compound typically having a molecular weight of less than about 1000. In some embodiments, a small molecule is an organic compound, with a size on the order of 1 nm. In some embodiments, small molecule drugs of the invention encompass oligopeptides and other biomolecules having a molecular weight of less than about 1000.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

The terms “decrease,” “reduce,” “reduced”, “reduction”, “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a S-fold or at least about a 10-fold increase, or any increase between 2-fold and 10-fold or greater as compared to a reference level.

As used herein, the term “modulate” includes up-regulation and down-regulation, e.g., enhancing or inhibiting a response.

A “radiopharmaceutical agent,” as defined herein, refers to a pharmaceutical agent which contains at least one radiation-emitting radioisotope. Radiopharmaceutical agents are routinely used in nuclear medicine for the diagnosis and/or therapy of various diseases. The radiolabelled pharmaceutical agent, for example, a radiolabelled antibody, contains a radioisotope (RI) which serves as the radiation source. As contemplated herein, the term “radioisotope” includes metallic and non-metallic radioisotopes. The radioisotope is chosen based on the medical application of the radiolabeled pharmaceutical agents. When the radioisotope is a metallic radioisotope, a chelator is typically employed to bind the metallic radioisotope to the rest of the molecule. When the radioisotope is a non-metallic radioisotope, the non-metallic radioisotope is typically linked directly, or via a linker, to the rest of the molecule.

For purposes of this invention, the chemical elements are identified in accordance with the Periodic Table of the Elements, CAS version, Handbook of Chemistry and Physics, 67th Ed., 1986-87, inside cover.

### COMPOUNDS OF THE INVENTION

In some embodiments, provided herein is a compound of Formula (I):

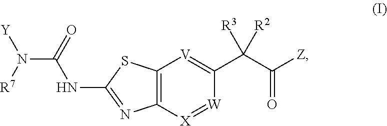

or a pharmaceutically acceptable salt thereof;
wherein:
   Q is O or S;
   V, W, and X are independently N or CR¹; provided that at least one of V, W, and X is CR¹;
   Y is (C₁-C₆)alkyl, (C₃-C₈)cycloalkyl, 4- to 7-membered heterocycloalkyl, phenyl, or 5- or 6-membered heteroaryl; each of which is optionally substituted with one, two, or three substituents selected independently for each occurrence from fluoro, chloro, cyano, hydroxyl, (C₁-C₆)alkyl, (C₃-C₈)cycloalkyl, (C₁-C₆)haloalkyl, (C₁-C₆)hydroxyalkyl, (C₁-C₆)alkoxyalkyl, and phenyl; each of which substituents is optionally substituted with one, two, or three substituents selected independently for each occurrence from fluoro, (C₁-C₆)alkyl, (C₁-C₆)haloalkyl, (C₁-C₆)hydroxyalkyl, and (C₁-C₆)alkoxyalkyl;
   Z is NR⁴R⁵ or OR⁶;
   R¹ is independently for each occurrence H, (C₁-C₆)alkyl, (C₁-C₆)haloalkyl, (C₁-C₆)hydroxyalkyl, (C₁-C₆)alkoxyalkyl, fluoro, chloro, cyano, hydroxyl, or (C₁-C₆)alkoxy;
   R² is (C₁-C₆)haloalkyl, (C₁-C₆)alkyl, or (C₃-C₈)cycloalkyl;
   R³ is hydroxyl, (C₁-C₆)alkyl, (C₁-C₆)alkoxyalkyl, (C₁-C₆)hydroxyalkyl, NR¹¹R¹², NC(O)R¹³, OC(O)NR¹¹R¹², or NR¹⁴SO₂R¹⁵; or R² and R³ together with the carbon atom to which they are attached form a (C₃-C₈)cycloalkyl or a 4- to 7-membered heterocycloalkyl; wherein (C₃-C₈)cycloalkyl or 4- to 7-membered heterocycloalkyl is optionally substituted with one, two, or three substituents selected independently for each occurrence from fluoro, chloro, cyano, hydroxyl, (C₁-C₆)alkyl. (C₁-C₆)haloalkyl. (C₁-C₆)hydroxyalkyl, and (C₁-C₆)alkoxyalkyl;
   R⁴ is hydrogen, (C₁-C₆)alkyl, (C₃-C₈)cycloalkyl, 4- to 7-membered heterocycloalkyl, phenyl, or 5- or 6-membered heteroaryl; wherein (C₁-C₆)alkyl is optionally substituted with one, two or three substituents selected independently for each occurrence from R^{4a}, halo, hydroxyl, cyano, carboxy, (C₁-C₆)alkylsulfonyl, (C₁-C₆)alkylcarbonyl, C(O)NR¹¹R¹², NR¹¹R¹², OC(O)NR¹¹R¹² and (C₁-C₆)alkoxy; and wherein (C₃-C₈)cycloalkyl, 4- to 7-membered heterocycloalkyl, phenyl, or 5- or 6-membered heteroaryl is optionally substituted with one, two, or three substituents selected independently for each occurrence from fluoro, chloro, cyano, hydroxyl, 5- to 10-membered heteroaryl, (C₁-C₆)alkyl, (C₁-C₆)haloalkyl, (C₁-C₆)hydroxyalkyl, (C₁-C₆)alkoxyalkyl, (C₁-C₆)alkylsulfonyl, (C₁-C₆)alkylcarbonyl C(O)NR¹¹R¹², NR¹¹R¹², NC(O)OR¹³, NC(O)R¹³, OC(O)NR¹¹R¹², and SO₂NR¹¹R¹².
   R^{4a} is (C₃-C₈)cycloalkyl, phenyl, 5- to 10-membered heteroaryl, or 4- to 7-membered heterocycloalkyl, each of which is optionally substituted with one, two, or three substituents selected independently for each occurrence from fluoro, chloro, cyano, hydroxyl, (C₁-C₆)alkyl, (C₁-C₆)haloalkyl, (C₁-C₆)hydroxyalkyl, (C₁-C₆)alkoxyalkyl, and (C₁-C₆)alkylsulfonyl;
   R⁵ is hydrogen or (C₁-C₆)alkyl; or R⁴ and R⁵ taken together with the nitrogen atom to which they are attached form an optionally 4- to 7-membered heterocycloalkyl: wherein 4- to 7-membered heterocycloalkyl is optionally substituted with one, two, or three substituents selected independently for each occurrence from fluoro, chloro, cyano, hydroxyl, carboxyl, 5- to 10-membered heteroaryl, (C₁-C₆)alkyl, (C₁-C₆)haloalkyl, (C₁-C₆)hydroxyalkyl, (C₁-C₆)alkoxyalkyl, (C₁-C₆)carboxyalkyl, (C₁-C₆)alkylsulfonyl, NR¹⁴SO₂R¹⁵, and C(O)NR¹¹R¹²;
   R⁶ is hydrogen or (C₁-C₆)alkyl;
   R⁷ is hydrogen or (C₁-C₆)alkyl; or R⁷ and Y taken together with the nitrogen atom to which they are attached form a 4- to 7-membered heterocycloalkyl;
   R¹¹ and R¹² are independently for each occurrence selected from hydrogen, (C₁-C₆)alkyl, and (C₃-C₈)cycloalkyl; or R¹¹ and R¹² taken together with the atoms to which they are attached form a 4- to 7-membered heterocycloalkyl;
   R¹³ is independently for each occurrence selected from (C₁-C₆)alkyl and (C₃-C₈)cycloalkyl; and
   R¹⁴ is independently for each occurrence selected from hydrogen, (C₁-C₆)alkyl, and (C₃-C₈)cycloalkyl, and R¹⁵ is independently for each occurrence selected from (C₁-C₆)alkyl and (C₃-C₈)cycloalkyl; or R¹⁴ and R¹⁵ taken together with the atoms to which they are attached form a 4- to 7-membered heterocycloalkyl.

In some embodiments, Q is O. In other embodiments, Q is S.

In more particular embodiments, provided herein is a compound of Formula (I):

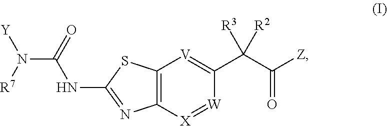

or a pharmaceutically acceptable salt thereof;
wherein:
   V, W, and X are independently N or CR¹; provided that at least one of V, W, and X is CR¹:
   Y is (C₁-C₆)alkyl, (C₃-C₈)cycloalkyl, 4- to 7-membered heterocycloalkyl, phenyl, or 5- or 6-membered heteroaryl; each of which is optionally substituted with one, two, or three substituents selected independently for each occurrence from fluoro, chloro, cyano, hydroxyl, (C₁-C₆)alkyl, (C₃-C₈)cycloalkyl, (C₁-C₆)haloalkyl, (C₁-C₆)hydroxyalkyl, (C₁-C₆)alkoxyalkyl, and phenyl: each of which substituents is optionally substituted with one, two, or three substituents selected independently for each occurrence from fluoro, (C₁-C₆)haloalkyl, (C₁-C₆)hydroxyalkyl, and (C₁-C₆)alkoxyalkyl;
   Z is NR⁴R⁵ or OR⁶;
   R¹ is independently for each occurrence H, (C₁-C₆)alkyl, (C₁-C₆)haloalkyl, (C₁-C₆)hydroxyalkyl, (C₁-C₆)alkoxyalkyl, fluoro, chloro, cyano, hydroxyl, or (C₁-C₆)alkoxy;
   R² is (C₁-C₆)haloalkyl, (C₁-C₆)alkyl, or (C₃-C₈)cycloalkyl;
   R² is hydroxyl, (C₁-C₆)alkoxyalkyl, or —NH₂; or R² and R³ together with the carbon atom to which they are attached form a (C₃-C₈)cycloalkyl or a 4- to 7-membered heterocycloalkyl: wherein (C₃-C₈)cycloalkyl or 4- to 7-membered heterocycloalkyl is optionally substituted with one, two, or three substituents selected independently for each occurrence from fluoro, chloro, cyano, hydroxyl, (C₁-C₆)alkyl, (C₁-C₆)haloalkyl, (C₁-C₆)hydroxyalkyl, and (C₁-C₆)alkoxyalkyl;
   R⁴ is hydrogen, (C₁-C₆)alkyl, (C₁-C₆)cycloalkyl, 4- to 7-membered heterocycloalkyl, phenyl, or 5- or 6-membered heteroaryl; wherein (C₁-C₆)alkyl is optionally substituted with one, two or three substituents selected independently for each occurrence from R^{4a}, halo, hydroxyl, cyano, carboxy, (C₁-C₆)alkylsulfonyl, and (C₁-C₆)alkoxy; and wherein (C₃-C₈)cycloalkyl, 4- to 7-membered heterocycloalkyl, phenyl, or 5- or 6-membered heteroaryl is optionally substituted with one, two, or three substituents selected independently for each occurrence from fluoro, chloro, cyano, hydroxyl, (C₁-C₆)alkyl, (C₁-C₆)haloalkyl, (C₁-C₆)hydroxyalkyl, and (C₁-C₆)alkoxyalkyl;
   R^{4a} is (C₃-C₈)cycloalkyl, phenyl, or 4- to 7-membered heterocycloalkyl, each of which is optionally substituted with one, two, or three substituents selected independently for each occurrence from fluoro, chloro, cyano, hydroxyl, (C₁-C₆)alkyl, (C₁-C₆)haloalkyl, (C₁-C₆)hydroxyalkyl, and (C₁-C₆)alkoxyalkyl;
   R⁵ is hydrogen or (C₁-C₆)alkyl; or R⁴ and R⁵ taken together with the nitrogen atom to which they are attached form an optionally 4- to 7-membered heterocycloalkyl; wherein 4- to 7-membered heterocycloalkyl is optionally substituted with one, two, or three substituents selected independently for each occurrence from fluoro, chloro, cyano, hydroxyl, carboxyl, (C₁-C₆)alkyl, (C₁-C₆)haloalkyl, (C₁-C₆)hydroxyalkyl, and (C₁-C₆)alkoxyalkyl; and
   R⁶ is hydrogen or (C₁-C₆)alkyl; and
   R⁷ is hydrogen or (C₁-C₆)alkyl; or R⁷ and Y taken together with the nitrogen atom to which they are attached form a 4- to 7-membered heterocyloalkyl.

In some embodiments, provided here in is a compound of Formula (I):

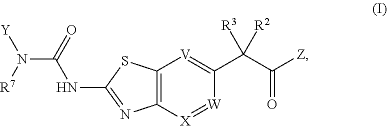

or a pharmaceutically acceptable salt thereof;
wherein;
   V, W, and X are independently N or CR¹; provided that at least one of V, W, and X is CR¹;
   Y is (C₁-C₆)alkyl, (C₃-C₆)cycloalkyl, 4- to 7-membered heterocycloalkyl, phenyl, or 5- or 6-membered heteroaryl, each of which is optionally substituted with one, two, or three substituents selected independently for each occurrence from fluoro, chloro, cyano, hydroxyl, (C₁-C₆)alkyl, (C₁-C₆)haloalkyl, (C₁-C₆)hydroxyalkyl, (C₁-C₆)alkoxyalkyl, or phenyl; Z is NR⁴R⁵ or OR⁶;
   R¹ is independently for each occurrence H, (C₁-C₆)alkyl, (C₁-C₆)haloalkyl, (C₁-C₆)hydroxyalkyl, (C₁-C₆)alkoxyalkyl, fluoro, chloro, cyano, hydroxyl, or (C₁-C₆)alkoxy;
   R² is (C₁-C₆)haloalkyl or (C₁-C₆)alkyl;
   R³ is hydroxyl; or R² and R³ together with the carbon atom to which they are attached form a (C₃-C₈)cycloalkyl or a 4- to 7-membered heterocycloalkyl; wherein (C₃-C₈)cycloalkyl or 4- to 7-membered heterocycloalkyl is optionally substituted with one, two, or three substituents selected independently for each occurrence from fluoro, chloro, cyano, hydroxyl, (C₁-C₆)alkyl, (C₁-C₆)haloalkyl, (C₁-C₆)hydroxyalkyl, and (C₁-C₆)alkoxyalkyl;
   R⁴ is hydrogen, (C₁-C₆)alkyl, (C₃-C₈)cycloalkyl, 4- to 7-membered heterocycloalkyl, phenyl, or 5- or 6-membered heteroaryl; wherein (C₁-C₆)alkyl is optionally substituted with one, two or three substituents selected independently for each occurrence from R^{4a}, halo, hydroxyl, and (C₁-C₆)alkoxy; and (C₃-C₈)cycloalkyl, 4- to 7-membered heterocycloalkyl, phenyl, or 5- or 6-membered heteroaryl is optionally substituted with one, two, or three substituents selected independently for each occurrence from fluoro, chloro, cyano, hydroxyl, (C₁-C₆)alkyl, (C₁-C₆)haloalkyl, (C₁-C₆)hydroxyalkyl, and (C₁-C₆)alkoxyalkyl;
   R^{4a} is (C₃-C₈)cycloalkyl, phenyl, or 4- to 7-membered heterocycloalkyl, each of which is optionally substituted with one, two, or three substituents selected independently for each occurrence from fluoro, chloro, cyano, hydroxyl, (C₁-C₆)alkyl, (C₁-C₆)haloalkyl, (C₁-C₆)hydroxyalkyl, and (C₁-C₆)alkoxyalkyl;
   R³ is hydrogen or (C₁-C₆)alkyl; or R⁴ and R⁵ taken together with the nitrogen atom to which they are attached form an optionally 4- to 7-membered heterocycloalkyl; wherein 4- to 7-membered heterocycloalkyl is optionally substituted with one, two, or three substituents selected independently for each occurrence from fluoro, chloro, cyano, hydroxyl, (C₁-C₆)alkyl, (C₁-C₆)haloalkyl, (C₁-C₆)hydroxyalkyl, and (C₁-C₆)alkoxyalkyl; and
   R⁶ is hydrogen or (C₁-C₆)alkyl; and
   R⁷ is (C₁-C₆)alkyl, or R⁷ and Y taken together with the nitrogen atom to which they are attached form a 4- to 7-membered heterocyloalkyl.

In more particular embodiments, the compound has the structure of formula Ia:

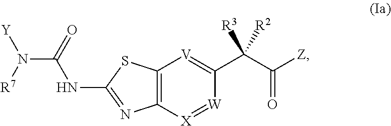

or a pharmaceutically acceptable salt thereof, or alternatively the structure of formula Ib:

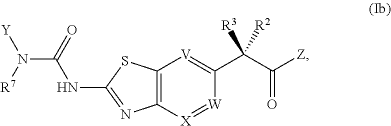

or a pharmaceutically acceptable salt thereof.

In other embodiments, provided herein is a compound of Formula (IIa):

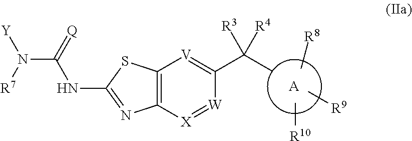

or a pharmaceutically acceptable salt thereof;
wherein:
A is 5- to 10-membered heteroaryl;
Q is S or O;
V, W, and X are independently N or CR¹, provided that at least one of V, W, and X is CR¹;
Y is (C₁-C₆)alkyl, (C₃-C₈)cycloalkyl, 4- to 7-membered heterocycloalkyl, phenyl, or 5- or 6-membered heteroaryl; each of which is optionally substituted with one, two, or three substituents selected independently for each occurrence from fluoro, chloro, cyano, hydroxyl, (C₁-C₆)alkyl, (C₃-C₈)cycloalkyl, (C₁-C₆)haloalkyl, (C₁-C₆)hydroxyalkyl, (C₁-C₆)alkoxyalkyl, and phenyl; each of which substituents is optionally substituted with one, two, or three substituents selected independently for each occurrence from fluoro, (C₁-C₆)alkyl, (C₁-C₆)haloalkyl, (C₁-C₆)hydroxyalkyl, and (C₁-C₆)alkoxyalkyl;
R¹ is independently for each occurrence H, (C₁-C₆)alkyl, (C₁-C₆)haloalkyl, (C₁-C₆)hydroxyalkyl, (C₁-C₆)alkoxyalkyl, fluoro, chloro, cyano, hydroxyl, or (C₁-C₆)alkoxy;
R² is (C₁-C₆)haloalkyl, (C₁-C₆)alkyl, or (C₃-C₈)cycloalkyl;
R³ is hydroxyl, (C₁-C₆)alkyl, (C₁-C₆)alkoxyalkyl, (C₁-C₆)hydroxyalkyl, NR¹¹R¹², NC(O)R¹³, OC(O)NR¹¹R¹², NR¹¹SO₂R¹²; or R² and R³ together with the carbon atom to which they are attached form a (C₃-C₈)cycloalkyl or a 4- to 7-membered heterocycloalkyl; wherein (C₃-C₈)cycloalkyl or 4- to 7-membered heterocycloalkyl is optionally substituted with one, two, or three substituents selected independently for each occurrence from fluoro, chloro, cyano, hydroxyl, (C₁-C₆)alkyl. (C₁-C₆)haloalkyl, (C₁-C₆)hydroxyalkyl, and (C₁-C₆)alkoxyalkyl;
R⁷ is hydrogen or (C₁-C₆)alkyl; or R⁷ and Y taken together with the nitrogen atom to which they are attached form a 4- to 7-membered heterocycloalkyl;
R⁸, R⁹ and R¹⁰ are independently for each occurrence H, (C₁-C₆)alkyl, or carboxy; wherein each (C₁-C₆)alkyl is optionally substituted with one, two, or three substituents selected independently for each occurrence from the group consisting of (C₁-C₆)alkoxy, hydroxy, fluoro, chloro, cyano, and NR¹¹R¹²;
R¹¹ and R¹² are independently for each occurrence selected from hydrogen, (C₁-C₆)alkyl, and (C₁-C₆)cycloalkyl; or R¹¹ and R¹² taken together with the atoms to which they are attached form a 4- to 7-membered heterocycloalkyl;
R¹³ is independently for each occurrence selected from (C₁-C₆)alkyl and (C₃-C₈)cycloalkyl; and
R¹⁴ is independently for each occurrence selected from hydrogen, (C₁-C₆)alkyl, and (C₃-C₈)cycloalkyl, and R¹⁵ is independently for each occurrence selected from (C₁-C₆)alkyl and (C₁-C₆)cycloalkyl; or R¹⁴ and R¹⁵ taken together with the atoms to which they are attached form a 4- to 7-membered heterocycloalkyl.

In certain embodiment, A is a 5- or 6-membered heteroaryl. In some embodiments, A comprises 1, 2, 3, or 4 nitrogen atoms. In more particular embodiments, A is diazolyl, triazolyl, imidazolyl, tetrazolyl, oxadiazolyl, thiadiazole, diazole, pyridazinyl, or pyrazinyl.

In some embodiments, R⁸, R⁹ and R¹⁰ are independently for each occurrence H, (C₁-C₆)alkyl, or carboxy; and each (C₁-C₆)alkyl is optionally substituted with one, two, or three instances of fluoro.

In some embodiments, Q is O, while in other embodiments, Q is S.

In some embodiments, provided herein is a compound of Formula (IIb):

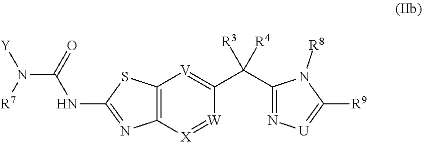

or a pharmaceutically acceptable salt thereof;
wherein:
   V, W, and X are independently N or CR¹; provided that at least one of V, W, and X is CR¹;
   Y is (C₁-C₆)alkyl, (C₃-C₈)cycloalkyl, 4- to 7-membered heterocycloalkyl, phenyl, or 5- or 6-membered heteroaryl; each of which is optionally substituted with one, two, or three substituents selected independently for each occurrence from fluoro, chloro, cyano, hydroxyl, (C₁-C₆)alkyl, (C₃-C₈)cycloalkyl, (C₁-C₆)haloalkyl, (C₁-C₆)hydroxyalkyl, (C₁-C₆)alkoxyalkyl, and phenyl; each of which substituents is optionally substituted with one, two, or three substituents selected independently for each occurrence from fluoro, (C₁-C₆)haloalkyl, (C₁-C₆)hydroxyalkyl, and (C₁-C₆)alkoxyalkyl;
   U is N or CR¹⁰;
   R¹ is independently for each occurrence H, (C₁-C₆)alkyl, (C₁-C₆)haloalkyl, (C₁-C₆)hydroxyalkyl. (C₁-C₆)alkoxyalkyl, fluoro, chloro, cyano, hydroxyl, or (C₁-C₆)alkoxy;
   R² is (C₁-C₆)haloalkyl, (C₁-C₆)alkyl, or (C₁-C₆)cycloalkyl;
   R³ is hydroxyl, (C₁-C₆)alkoxyalkyl, or —NH₂; or R² and R³ together with the carbon atom to which they are attached form a (C₃-C₈)cycloalkyl or a 4- to 7-membered heterocycloalkyl; wherein (C₃-C₈)cycloalkyl or 4- to 7-membered heterocycloalkyl is optionally substituted with one, two, or three substituents selected independently for each occurrence from fluoro, chloro, cyano, hydroxyl, (C₁-C₆)alkyl, (C₁-C₆)haloalkyl, (C₁-C₆)hydroxyalkyl, and (C₁-C₆)alkoxyalkyl;
   R⁷ is hydrogen or (C₁-C₆)alkyl; or R⁷ and Y taken together with the nitrogen atom to which they are attached form a 4- to 7-membered heterocyloalkyl;
   R⁸, R⁹ and R¹⁰ are independently for each occurrence H or (C₁-C₆)alkyl, wherein each (C₁-C₆)alkyl is optionally substituted with one, two, or three substituents selected independently for each occurrence from the group consisting of hydroxy, (C₁-C₆)hydroxyalkyl, (C₁-C₆)alkoxyalkyl, (C₁-C₆)alkoxy, fluoro, chloro, cyano, or NR¹¹R¹², and
   R¹¹ and R¹² are each independently hydrogen or (C₁-C₆)alkyl; or R¹¹ and R¹² taken together with the nitrogen atom to which they are attached form a 4- to 7-membered heterocycloalkyl.

In some embodiments, R⁸ is H.

In some embodiments, R⁹ is (C₁-C₆)alkyl, preferably methyl.

In some embodiments, U is N, while in other embodiments, U is CR¹⁰. In more particular embodiments, R¹⁰ is H.

In some embodiments, V, W, and X are each CR¹. In other embodiments, one of V, W and X is N. For example, in some embodiments, V is N, and W and X are each CR¹. In other embodiments, W is N; and V and X are each CR¹. In still other embodiments, X is N; and V and W are each CR¹. In other embodiments, two of V, W, and X are N. For example, in some embodiments, V and W are each N, and X is CR¹.

In some embodiments, R¹ is independently for each occurrence selected from hydrogen, methyl, trifluoromethyl, fluoro, and methoxy. In more particular embodiments, one and only one instance of R¹ is selected from methyl, trifluoromethyl, fluoro, and methoxy; and the remaining instances of R¹ are hydrogen. In other embodiments, each occurrence of R¹ is hydrogen.

In certain embodiments, Y is optionally substituted with fluoro or chloro. In some embodiments, Y is optionally substituted phenyl. In more particular embodiments, Y is phenyl optionally substituted with 1 or 2 substituents independently selected from fluoro and cyano. In still more particular embodiments, Y is 2,3-difluorophenyl, 2,4-difluorophenyl, 4-fluorophenyl, or 4-cyanophenyl. More preferably, Y is 4-fluoro phenyl. In some embodiments, Y is optionally substituted 2-pyridyl, such as 4-fluoro-2-pyridyl.

In other embodiments, Y is 5-thiazolyl or 5-isothiazolyl, each of which is optionally substituted with methyl.

In certain embodiments, Y is optionally substituted (C₃-C₈)cycloalkyl.

In some embodiments, Y is bicyclo[1.1.1]pent-1-yl or cyclopentyl, each of which is optionally substituted with one or two fluoro substituents.

In some embodiments, Y is methyl substituted with cyclopropyl, cyclobutyl, or cyclopentyl, each of which is optionally substituted with one or two substituents independently selected from fluoro, trifluoromethyl, and methyl.

In some embodiments, R² is (C₁-C₆)haloalkyl. In more particular embodiments, R² is (C₁-C₆)fluoroalkyl, preferably trifluoromethyl. In other embodiments, R² is difluoromethyl or 2,2,2-trifluoroethyl.

In some embodiments, R² is cyclopropyl.

In some embodiments, R² is (C₁-C₆)alkyl. In preferred embodiments, R² is methyl. In other embodiments, R² is ethyl.

In some embodiments, R³ is hydroxy. In other embodiments R³ is methoxy, and in still other embodiments, R³ is —NH₂. In further embodiments, R³ is NR¹¹R¹², NC(O)R¹³, OC(O)NR¹¹R¹², or NR¹¹SO₂R¹².

In some embodiments, R² and R³ together with the carbon atom to which they are attached form an optionally substituted (C₃-C₈)cycloalkyl, in some embodiments, R² and R³ together with the carbon atom to which they are attached form a cyclopentyl, cyclobutyl, or cyclopropyl.

In some embodiments, R² and R³ together with the carbon atom to which they are attached form an optionally substituted 4- to 7-membered heterocycloalkyl. In more particular embodiments, R² and R³, together with the carbon atom to which they are attached, form an optionally substituted 5-membered heterocycloalkyl. In still more particular embodiments, R² and R³ together with the carbon atom to which they are attached form a pyrrolidine or a tetrahydrofuran.

In certain embodiments, Z is NR⁴R⁵.

In some embodiments, R⁴ is hydrogen, while in other embodiments, R⁴ is (C₁-C₆)alkyl optionally substituted with one, two, or three substituents independently selected from fluoro, (C₁-C₆)alkylsulfonyl, cyano, carboxy, NR¹¹R¹², or C(O)NR¹¹R¹². In more particular embodiments, R⁴ is (C₁-C₆)alkyl optionally substituted with one, two, or three substituents independently selected from halo and R^{4a}.

In certain embodiments, R⁴ is (C₁-C₃)alkyl optionally substituted with fluoro, (C₁-C₆)alkylsulfonyl, cyano, or carboxy. In more particular embodiments, R⁴ is (C₁-C₆)alkyl substituted with one, two, or three instances of fluoro.

In certain embodiments, R⁴ is methyl, ethyl or isopropyl, each of which is optionally substituted with fluoro, carboxy, methylsulfonyl, or cyano, In some embodiments, R⁴ is ethyl or isopropyl.

In certain embodiments, R⁴ is (C₁-C₆)alkyl substituted with one, two, or three instances of R^{4a}. In more particular embodiments, R⁴ is methyl substituted with one, two, or three substituents independently selected from R^{4a}.

In some embodiments, R^{4a} is (C₃-C₈)cycloalkyl optionally substituted with one, two, or three substituents independently selected from halo, cyano, carboxy, hydroxyl, (C₁-C₆)alkyl, (C₁-C₆)haloalkyl, and (C₁-C₆)alkoxy. In some embodiments, R^{4a} is (C₃-C₈)cycloalkyl optionally substituted with fluoro. In some embodiments, R^{4a} is cyclopentyl optionally substituted with hydroxy. In other embodiments, R^{4a} is cyclopropyl substituted with carboxy. In other embodiments, R^{4a} is unsubstituted cyclopropyl.

In some embodiments, R^{4a} is phenyl optionally substituted with one, two, or three substituents independently selected from halo, hydroxyl, and (C₁-C₆)alkoxy. In some embodiments, R^{4a} is phenyl optionally substituted with one, two, or three instances of (C₁-C₆)alkoxy. In more particular embodiments, R^{4a} is phenyl optionally substituted with methoxy.

In some embodiments, R^{4a} is 4- to 7-membered heterocycloalkyl optionally substituted with one, two, or three substituents independently selected from halo, cyano, hydroxyl, (C₁-C₆)alkyl, (C₁-C₆)haloalkyl, and (C₁-C₆)alkoxy.

In some embodiments, R^{4a} is phenyl optionally substituted with one, two, or three substituents independently selected from halo, hydroxyl, and (C₁-C₆)alkoxy. More particularly, R^{4a} is phenyl optionally substituted with one, two, or three substituents independently selected from (C₁-C₆)alkoxy, such as methoxy.

In some embodiments, wherein R^{4a} is 4- to 7-membered heterocycloalkyl optionally substituted with one, two, or three substituents independently selected from halo, cyano, hydroxyl, (C₁-C₆)alkyl, (C₁-C₆)haloalkyl, and (C₁-C₆)alkoxy. In more particular embodiments, R^{4a} is tetrahydrofuranyl, oxetanyl, piperidinyl, or pyrrolidinyl, each of which is optionally substituted with one, two, or three substituents independently selected from halo, cyano, hydroxyl, (C₁-C₆)alkyl, (C₁-C₆)haloalkyl, and (C₁-C₆)alkoxy.

In some embodiments, R⁴ is (C₃-C₈)cycloalkyl optionally substituted with one, two, or three substituents independently selected from halo, hydroxyl, carboxy, (C₁-C₆)alkyl, and (C₁-C₆)alkoxy. In more particular embodiments, R⁴ is (C₃-C₈)cycloalkyl optionally substituted with one, two, or three substituents independently selected from halo, hydroxyl, and (C₁-C₆)alkoxy.

In more particular embodiments, (C₃-C₈)cycloalkyl is optionally substituted with one, two or three instances of hydroxyl. In other particular embodiments, (C₁-C₆)cycloalkyl is optionally substituted with one, two, or three instances of fluoro. In some embodiments, (C₃-C₆)cycloalkyl is optionally substituted with (C₁-C₆)alkylsulfonyl, (C₁-C₆)alkylcarbonyl C(O)NR¹¹R¹², NR¹¹R¹², NC(O)OR¹³, NC(O)R¹³, OC(O)NR¹¹R¹², or SO₂NR¹¹R¹².

In certain embodiments, R⁴ is (C₁-C₆)cycloalkyl, wherein (C₃-C₈)cycloalkyl is cyclopropyl, cyclobutyl, or cyclopentyl. In certain preferred embodiments, (C₃-C₈)cycloalkyl is cyclobutyl.

In some embodiments, R⁴ is 4- to 7-membered heterocycloalkyl optionally substituted with one, two, or three substituents independently selected from halo, hydroxyl, (C₁-C₆)alkoxy, carboxy, and (C₁-C₆)alkyl. In some embodiments, is R⁴ is 4- to 7-membered heterocycloalkyl optionally substituted with one, two, or three substituents independently selected from halo, hydroxyl, and (C₁-C₆)alkoxy. In more particular embodiments, R⁴ is oxetanyl, piperidinyl, pyrrolidinyl, or tetrahydrofuranyl, each of which is optionally substituted with one, two, or three substituents independently selected from hydroxyl and carboxyl, and in certain preferred embodiments, R⁴ is oxetane or tetrahydrofuran.

In some embodiments, R⁴ is phenyl.

In some embodiments, R⁴ is 5- or 6-membered heteroaryl, such as pyridyl or diazolyl. In some embodiments, R⁵ is hydrogen.

In some embodiments, R⁵ is hydrogen. In other embodiments, R⁵ is (C₁-C₆)alkyl. In more particular embodiments, R⁵ is ethyl.

In some embodiments, R⁴ and R⁵ taken together with the nitrogen atom to which they are attached form a 4- to 7-membered heterocycloalkyl optionally substituted with one, two, or three substituents independently selected from fluoro, hydroxy, carboxy, cyano, (C₁-C₆)alkyl, (C₁-C₆)alkoxycarbonyl, (C₁-C₆)alkylcarbonyl, (C₁-C₆)alkylsulfonyl, (C₁-C₆)fluoroalkyl, and (C₃-C₈)cycloalkyl. In other embodiments, R⁴ and R⁵ taken together with the nitrogen atom to which they are attached form a 4- to 7-membered heterocycloalkyl optionally substituted with 5- to 10-membered heteroaryl. (C₁-C₆)carboxyalkyl, (C₁-C₆)alkylsulfonyl, NR¹⁴SO₂R¹⁵, or C(O)NR¹¹R¹².

In more particular embodiments, R⁴ and R⁵ taken together with the nitrogen atom to which they are attached form azetidinyl, pyrrolidinyl, piperidinyl, piperazinyl, tetrahydropyranyl, morpholinyl, 3-azabicyclo[3.1.0]hexanyl, 2-oxa-6-aza-6-spiro[3.3]heptyl, 8-oxa-3-azabicyclo[3.2.1]oct-3-yl, and 1,7-diaza-1-indanyl. In certain preferred embodiments, R⁴ and R⁵ taken together with the nitrogen atom to which they are attached form azetidinyl.

In some embodiments, Z is OR⁶. In more particular embodiments, R⁶ is hydrogen. In alternative embodiments, R⁶ is (C₁-C₆)alkyl. For example, R⁶ may be methyl or ethyl. In some embodiments, R⁷ is hydrogen.

In other embodiments, R⁷ is (C₁-C₆)alkyl.

In still other embodiments, Y and R⁷ taken together with the nitrogen atom to which they are attached form a 4- to 7-membered heterocycloalkyl. For example, the 4- to 7-membered heterocycloalkyl may be piperidine.

In some embodiments, R¹¹ is hydrogen. In some embodiments, R¹² is hydrogen, while in other embodiments. R¹² is (C₁-C₆)alkyl.

In some embodiments, the compound has the following structure:

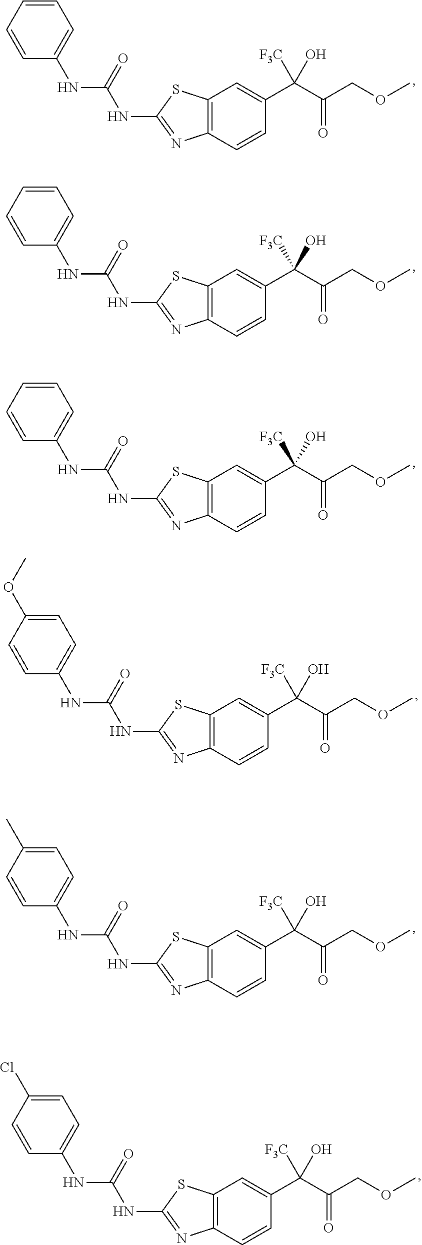 [SMILES: COCC(=O)C(O)(c1ccc2nc(NC(=O)Nc3ccccc3)sc2c1)C(F)(F)F ; COCC(=O)[C@](O)(c1ccc2nc(NC(=O)Nc3ccccc3)sc2c1)C(F)(F)F ; COCC(=O)[C@@](O)(c1ccc2nc(NC(=O)Nc3ccccc3)sc2c1)C(F)(F)F ; COCC(=O)C(O)(c1ccc2nc(NC(=O)Nc3ccc(OC)cc3)sc2c1)C(F)(F)F ; COCC(=O)C(O)(c1ccc2nc(NC(=O)Nc3ccc(C)cc3)sc2c1)C(F)(F)F ; COCC(=O)C(O)(c1ccc2nc(NC(=O)Nc3ccc(Cl)cc3)sc2c1)C(F)(F)F]

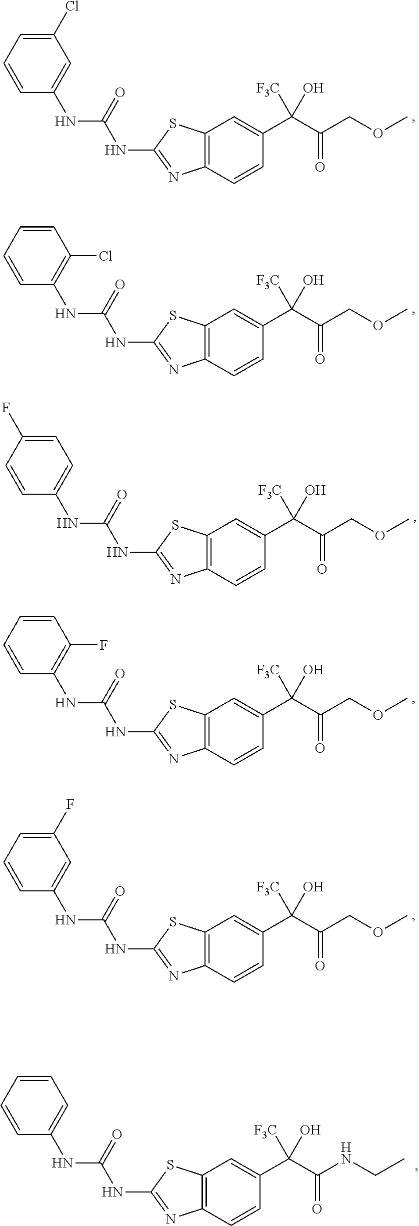 [SMILES: COCC(=O)C(O)(c1ccc2nc(NC(=O)Nc3cccc(Cl)c3)sc2c1)C(F)(F)F ; COCC(=O)C(O)(c1ccc2nc(NC(=O)Nc3ccccc3Cl)sc2c1)C(F)(F)F ; COCC(=O)C(O)(c1ccc2nc(NC(=O)Nc3ccc(F)cc3)sc2c1)C(F)(F)F ; COCC(=O)C(O)(c1ccc2nc(NC(=O)Nc3ccccc3F)sc2c1)C(F)(F)F ; COCC(=O)C(O)(c1ccc2nc(NC(=O)Nc3cccc(F)c3)sc2c1)C(F)(F)F ; CCNC(=O)C(O)(c1ccc2nc(NC(=O)Nc3ccccc3)sc2c1)C(F)(F)F]

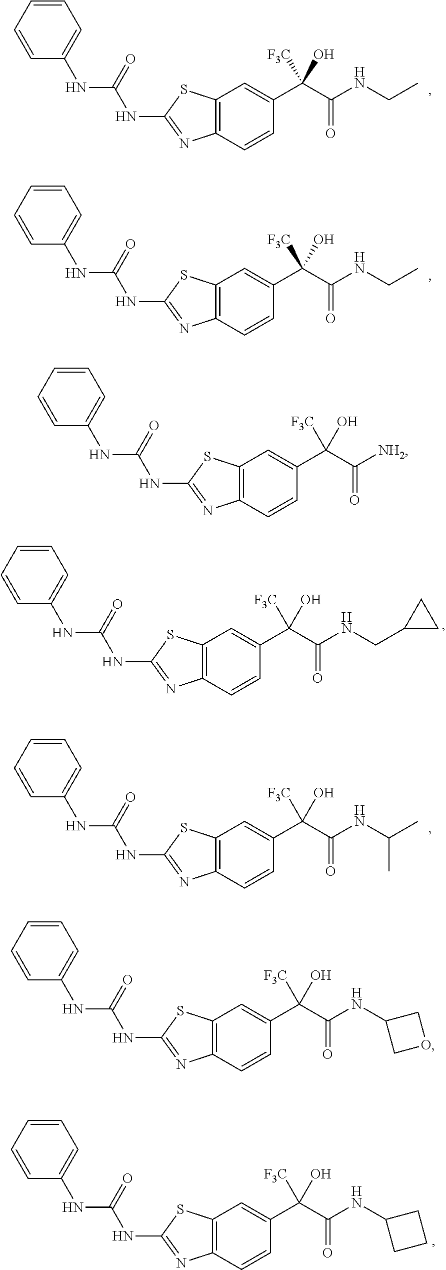

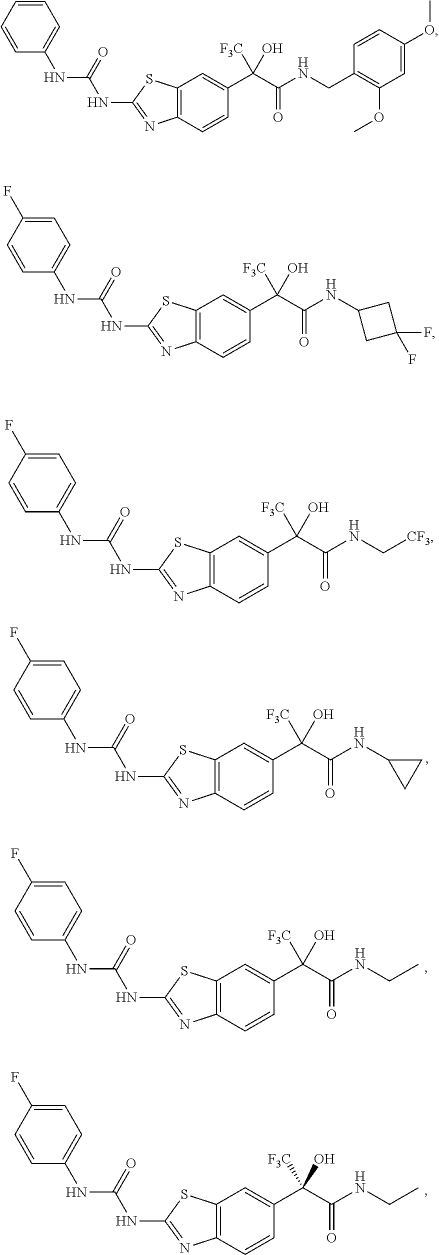 [SMILES: COc1ccc(CNC(=O)C(O)(c2ccc3nc(NC(=O)Nc4ccccc4)sc3c2)C(F)(F)F)c(OC)c1 ; O=C(Nc1ccc(F)cc1)Nc1nc2ccc(C(O)(C(=O)NC3CC(F)(F)C3)C(F)(F)F)cc2s1 ; O=C(Nc1ccc(F)cc1)Nc1nc2ccc(C(O)(C(=O)NCC(F)(F)F)C(F)(F)F)cc2s1 ; O=C(Nc1ccc(F)cc1)Nc1nc2ccc(C(O)(C(=O)NC3CC3)C(F)(F)F)cc2s1 ; CCNC(=O)C(O)(c1ccc2nc(NC(=O)Nc3ccc(F)cc3)sc2c1)C(F)(F)F ; CCNC(=O)[C@](O)(c1ccc2nc(NC(=O)Nc3ccc(F)cc3)sc2c1)C(F)(F)F]

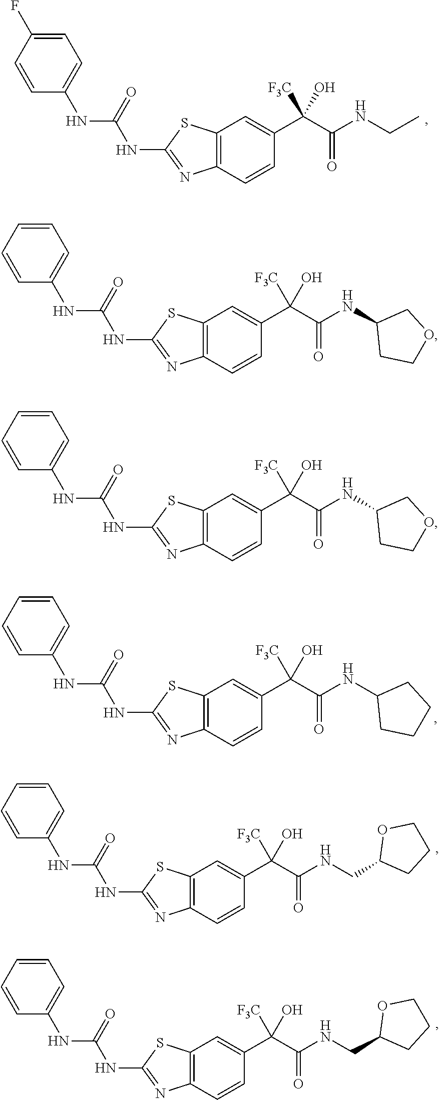 [SMILES: CCNC(=O)[C@@](O)(c1ccc2nc(NC(=O)Nc3ccc(F)cc3)sc2c1)C(F)(F)F ; O=C(Nc1ccccc1)Nc1nc2ccc(C(O)(C(=O)N[C@@H]3CCOC3)C(F)(F)F)cc2s1 ; O=C(Nc1ccccc1)Nc1nc2ccc(C(O)(C(=O)N[C@H]3CCOC3)C(F)(F)F)cc2s1 ; O=C(Nc1ccccc1)Nc1nc2ccc(C(O)(C(=O)NC3CCCC3)C(F)(F)F)cc2s1 ; O=C(Nc1ccccc1)Nc1nc2ccc(C(O)(C(=O)NC[C@H]3CCCO3)C(F)(F)F)cc2s1 ; O=C(Nc1ccccc1)Nc1nc2ccc(C(O)(C(=O)NC[C@@H]3CCCO3)C(F)(F)F)cc2s1]

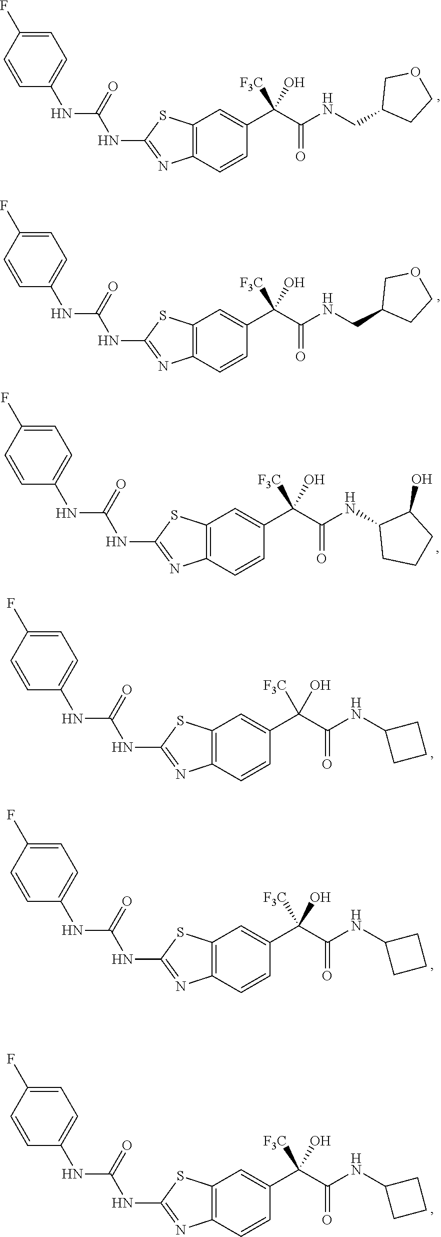 [SMILES: O=C(Nc1ccc(F)cc1)Nc1nc2ccc([C@](O)(C(=O)NC[C@H]3CCOC3)C(F)(F)F)cc2s1 ; O=C(Nc1ccc(F)cc1)Nc1nc2ccc([C@](O)(C(=O)NC[C@@H]3CCOC3)C(F)(F)F)cc2s1 ; O=C(Nc1ccc(F)cc1)Nc1nc2ccc([C@](O)(C(=O)N[C@H]3CCC[C@@H]3O)C(F)(F)F)cc2s1 ; O=C(Nc1ccc(F)cc1)Nc1nc2ccc(C(O)(C(=O)NC3CCC3)C(F)(F)F)cc2s1 ; O=C(Nc1ccc(F)cc1)Nc1nc2ccc([C@@](O)(C(=O)NC3CCC3)C(F)(F)F)cc2s1 ; O=C(Nc1ccc(F)cc1)Nc1nc2ccc([C@](O)(C(=O)NC3CCC3)C(F)(F)F)cc2s1]

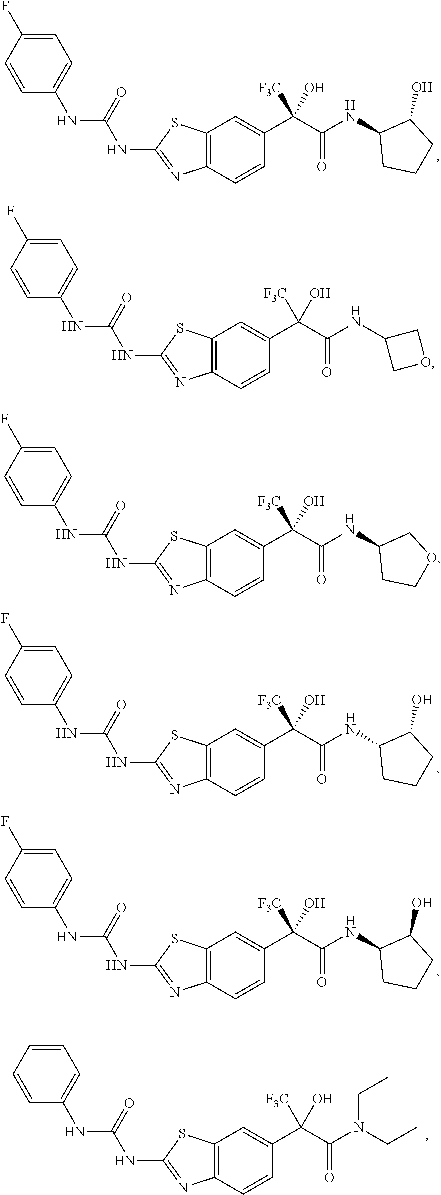 [SMILES: O=C(Nc1ccc(F)cc1)Nc1nc2ccc([C@](O)(C(=O)N[C@@H]3CCC[C@H]3O)C(F)(F)F)cc2s1 ; O=C(Nc1ccc(F)cc1)Nc1nc2ccc(C(O)(C(=O)NC3COC3)C(F)(F)F)cc2s1 ; O=C(Nc1ccc(F)cc1)Nc1nc2ccc([C@](O)(C(=O)N[C@@H]3CCOC3)C(F)(F)F)cc2s1 ; O=C(Nc1ccc(F)cc1)Nc1nc2ccc([C@](O)(C(=O)N[C@H]3CCC[C@H]3O)C(F)(F)F)cc2s1 ; O=C(Nc1ccc(F)cc1)Nc1nc2ccc([C@](O)(C(=O)N[C@@H]3CCC[C@@H]3O)C(F)(F)F)cc2s1 ; CCN(CC)C(=O)C(O)(c1ccc2nc(NC(=O)Nc3ccccc3)sc2c1)C(F)(F)F]

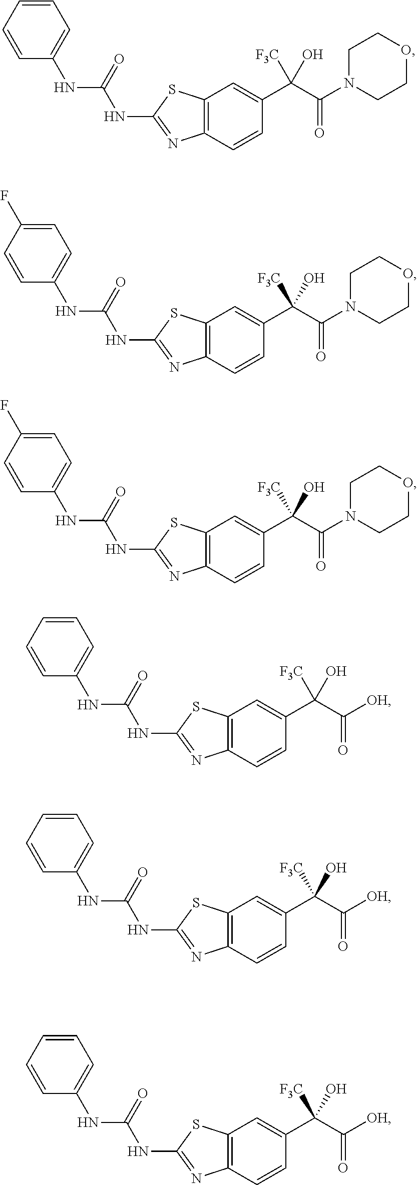 [SMILES: O=C(Nc1ccccc1)Nc1nc2ccc(C(O)(C(=O)N3CCOCC3)C(F)(F)F)cc2s1 ; O=C(Nc1ccc(F)cc1)Nc1nc2ccc([C@](O)(C(=O)N3CCOCC3)C(F)(F)F)cc2s1 ; O=C(Nc1ccc(F)cc1)Nc1nc2ccc([C@@](O)(C(=O)N3CCOCC3)C(F)(F)F)cc2s1 ; O=C(Nc1ccccc1)Nc1nc2ccc(C(O)(C(=O)O)C(F)(F)F)cc2s1 ; O=C(Nc1ccccc1)Nc1nc2ccc([C@@](O)(C(=O)O)C(F)(F)F)cc2s1 ; O=C(Nc1ccccc1)Nc1nc2ccc([C@](O)(C(=O)O)C(F)(F)F)cc2s1]

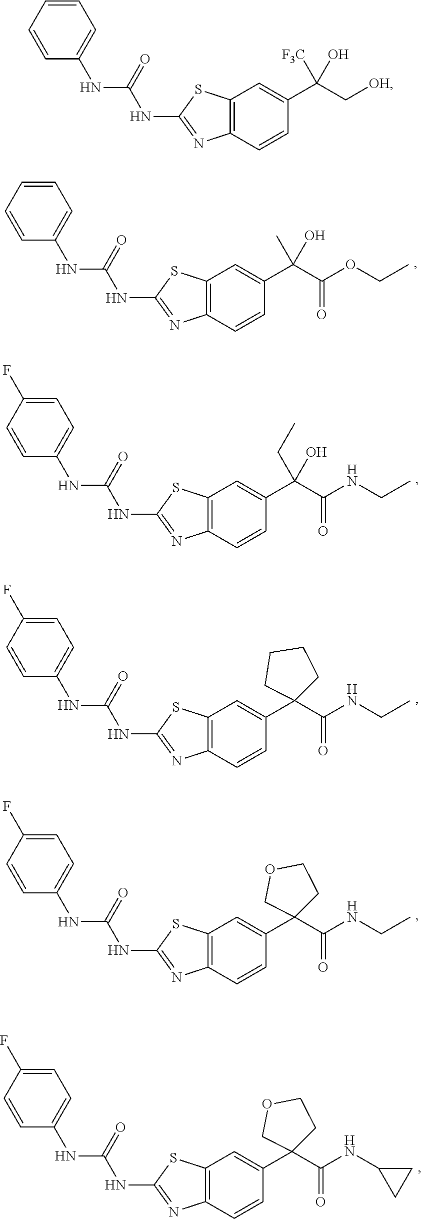 [SMILES: O=C(Nc1ccccc1)Nc1nc2ccc(C(O)(CO)C(F)(F)F)cc2s1 ; CCOC(=O)C(C)(O)c1ccc2nc(NC(=O)Nc3ccccc3)sc2c1 ; CCNC(=O)C(O)(CC)c1ccc2nc(NC(=O)Nc3ccc(F)cc3)sc2c1 ; CCNC(=O)C1(c2ccc3nc(NC(=O)Nc4ccc(F)cc4)sc3c2)CCCC1 ; CCNC(=O)C1(c2ccc3nc(NC(=O)Nc4ccc(F)cc4)sc3c2)CCOC1 ; O=C(Nc1ccc(F)cc1)Nc1nc2ccc(C3(C(=O)NC4CC4)CCOC3)cc2s1]

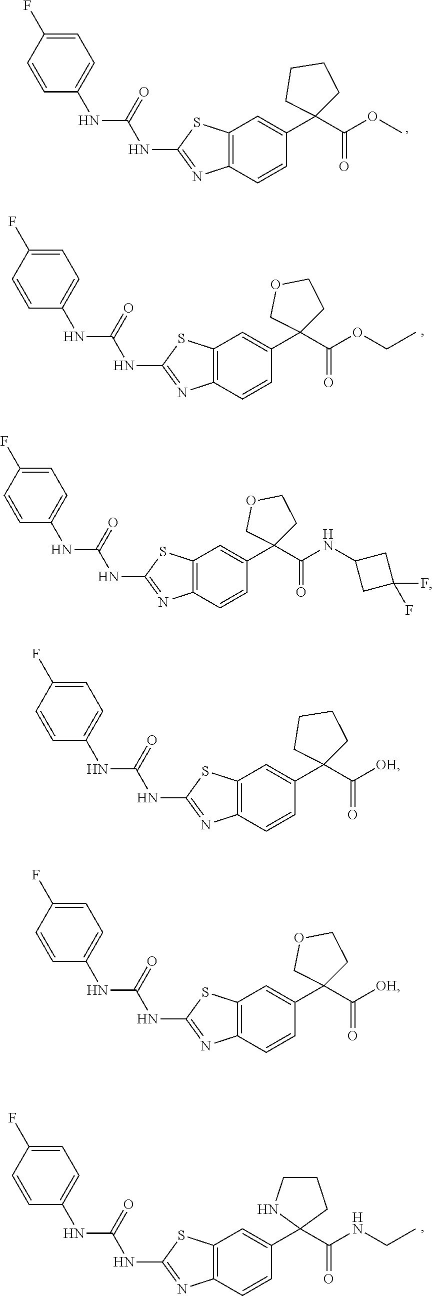 [SMILES: COC(=O)C1(c2ccc3nc(NC(=O)Nc4ccc(F)cc4)sc3c2)CCCC1 ; CCOC(=O)C1(c2ccc3nc(NC(=O)Nc4ccc(F)cc4)sc3c2)CCOC1 ; O=C(Nc1ccc(F)cc1)Nc1nc2ccc(C3(C(=O)NC4CC(F)(F)C4)CCOC3)cc2s1 ; O=C(Nc1ccc(F)cc1)Nc1nc2ccc(C3(C(=O)O)CCCC3)cc2s1 ; O=C(Nc1ccc(F)cc1)Nc1nc2ccc(C3(C(=O)O)CCOC3)cc2s1 ; CCNC(=O)C1(c2ccc3nc(NC(=O)Nc4ccc(F)cc4)sc3c2)CCCN1]

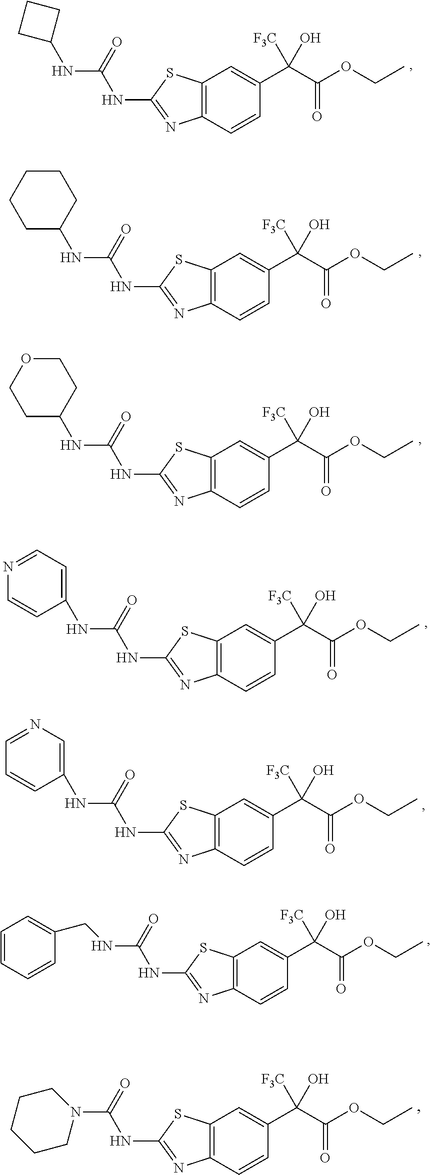

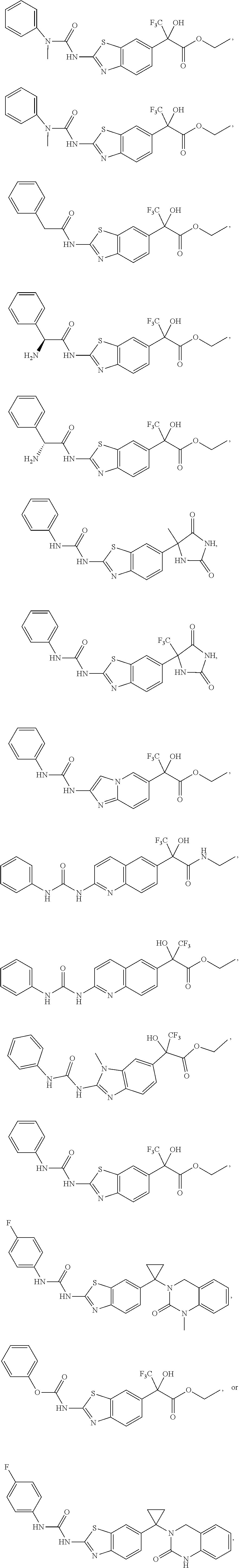

or a pharmaceutically acceptable salt thereof.

In other embodiments, the compound has the structure:

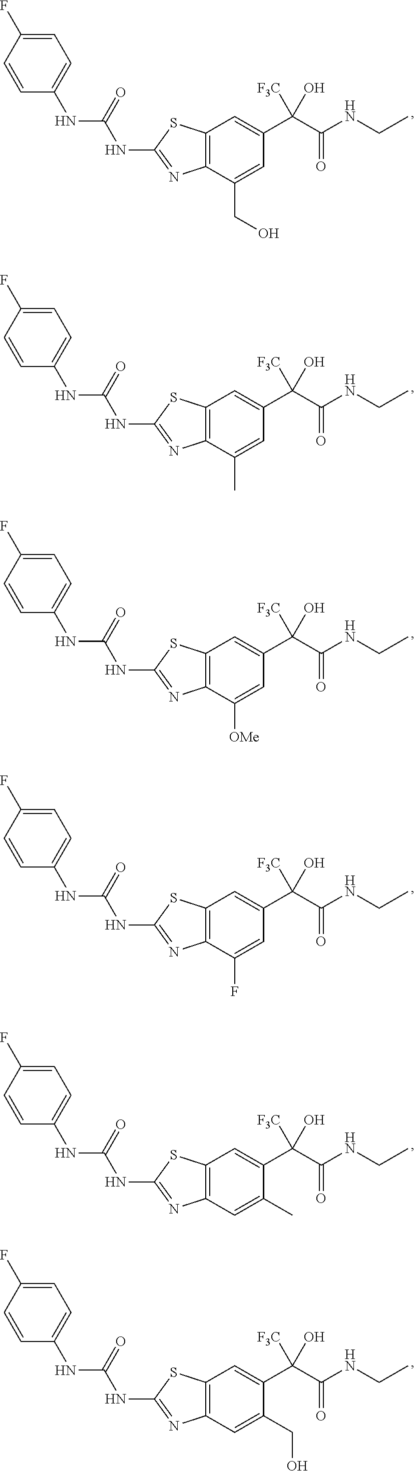 [SMILES: CCNC(=O)C(O)(c1cc(CO)c2nc(NC(=O)Nc3ccc(F)cc3)sc2c1)C(F)(F)F ; CCNC(=O)C(O)(c1cc(C)c2nc(NC(=O)Nc3ccc(F)cc3)sc2c1)C(F)(F)F ; CCNC(=O)C(O)(c1cc(OC)c2nc(NC(=O)Nc3ccc(F)cc3)sc2c1)C(F)(F)F ; CCNC(=O)C(O)(c1cc(F)c2nc(NC(=O)Nc3ccc(F)cc3)sc2c1)C(F)(F)F ; CCNC(=O)C(O)(c1cc2sc(NC(=O)Nc3ccc(F)cc3)nc2cc1C)C(F)(F)F ; CCNC(=O)C(O)(c1cc2sc(NC(=O)Nc3ccc(F)cc3)nc2cc1CO)C(F)(F)F]

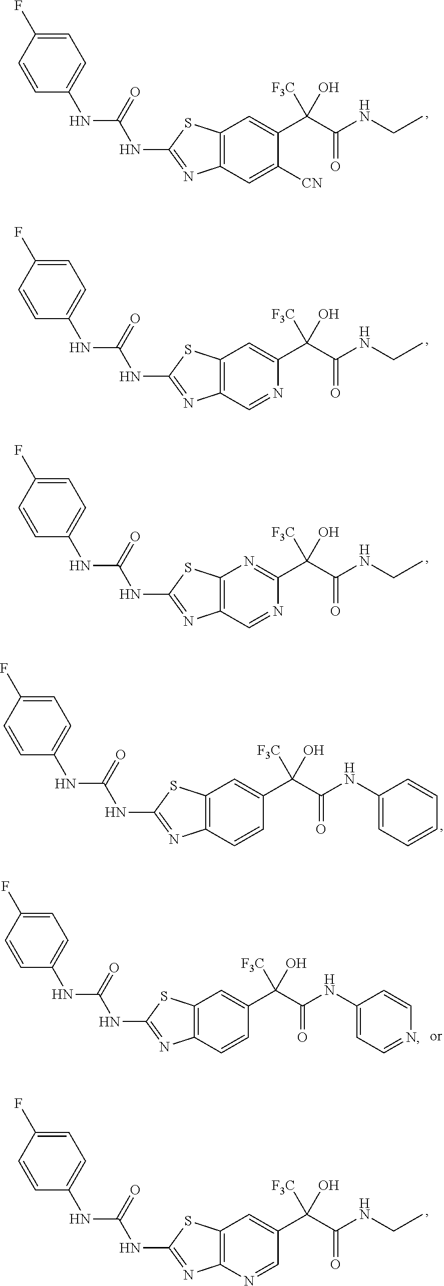

or a pharmaceutically acceptable salt thereof.

In still other embodiments, the compound has a structure selected from the following table:

| # | Structure |
|---|---|
| 87 | 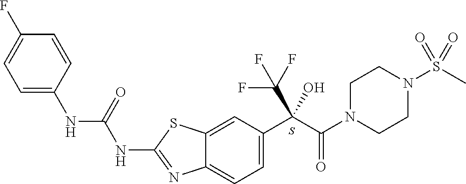 |
| 88 | 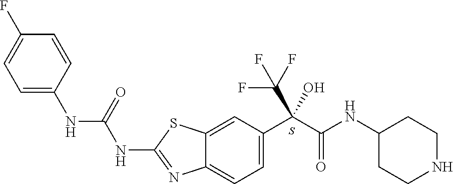 |
| 89 | 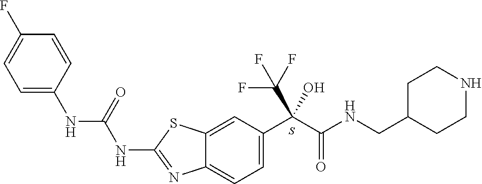 |
| 90 | 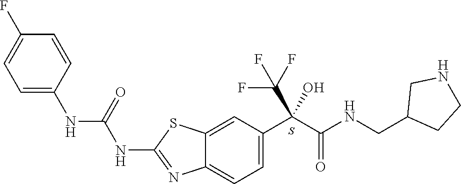 |
| 91 | 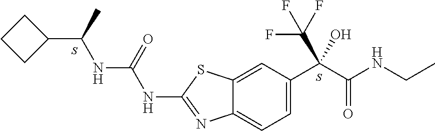 |
| 92 | 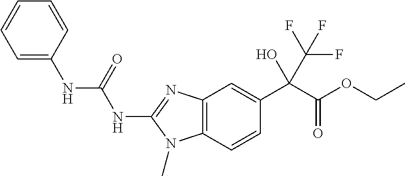 [SMILES: CCOC(=O)C(O)(c1ccc2c(c1)nc(NC(=O)Nc1ccccc1)n2C)C(F)(F)F] |
| 93 | 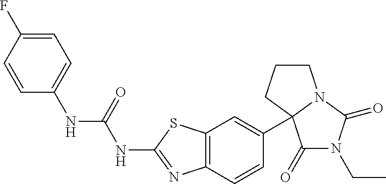 [SMILES: CCN1C(=O)N2CCCC2(c2ccc3nc(NC(=O)Nc4ccc(F)cc4)sc3c2)C1=O] |
| 94 | 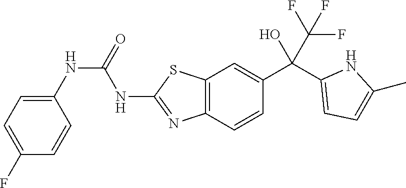 [SMILES: Cc1ccc(C(O)(c2ccc3nc(NC(=O)Nc4ccc(F)cc4)sc3c2)C(F)(F)F)[nH]1] |
| 95 | 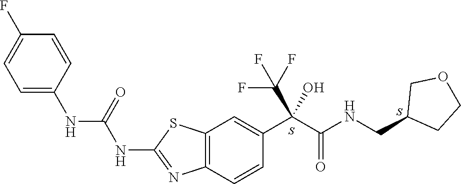 |
| 96 | 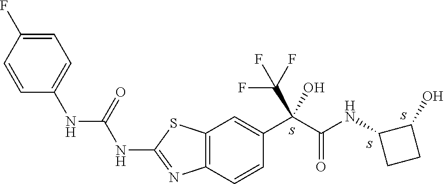 |
| 97 | 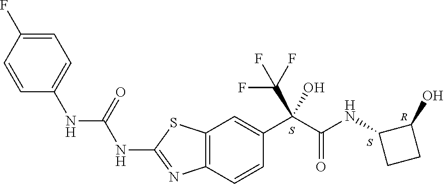 |
| 98 | 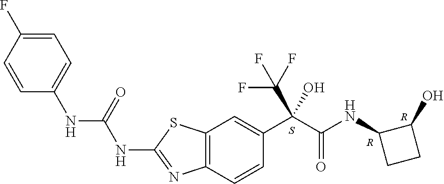 |
| 99 | 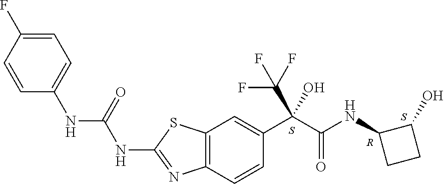 |
| 100 | 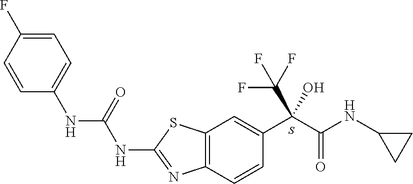 |
| 101 | 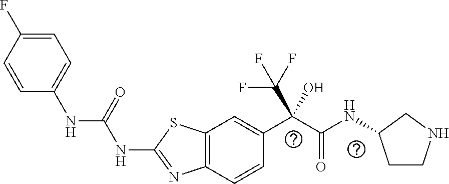 [SMILES: O=C(Nc1ccc(F)cc1)Nc1nc2ccc([C@](O)(C(=O)N[C@H]3CCNC3)C(F)(F)F)cc2s1] |
| 102 | 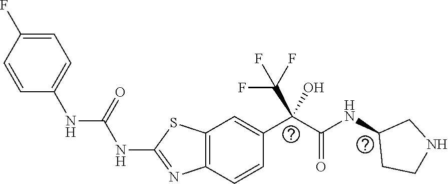 [SMILES: O=C(Nc1ccc(F)cc1)Nc1nc2ccc([C@](O)(C(=O)N[C@@H]3CCNC3)C(F)(F)F)cc2s1] |
| 103 | 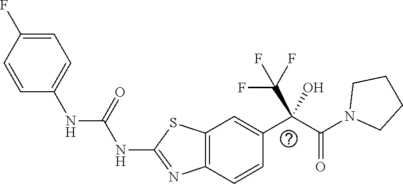 [SMILES: O=C(Nc1ccc(F)cc1)Nc1nc2ccc([C@](O)(C(=O)N3CCCC3)C(F)(F)F)cc2s1] |
| 104 | 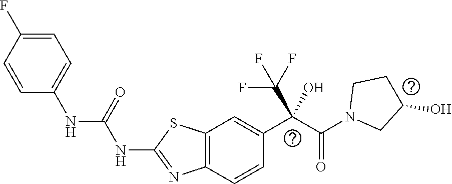 [SMILES: O=C(Nc1ccc(F)cc1)Nc1nc2ccc([C@](O)(C(=O)N3CC[C@H](O)C3)C(F)(F)F)cc2s1] |
| 105 | 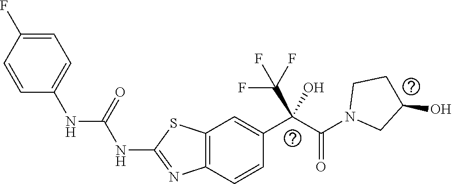 [SMILES: O=C(Nc1ccc(F)cc1)Nc1nc2ccc([C@](O)(C(=O)N3CC[C@@H](O)C3)C(F)(F)F)cc2s1] |
| 106 | 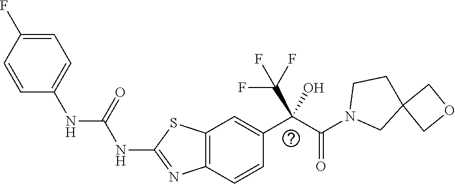 [SMILES: O=C(Nc1ccc(F)cc1)Nc1nc2ccc([C@](O)(C(=O)N3CCC4(COC4)C3)C(F)(F)F)cc2s1] |
| 107 | 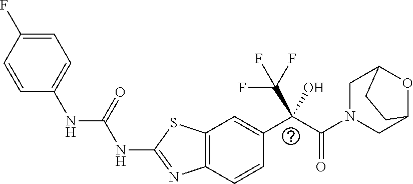 [SMILES: O=C(Nc1ccc(F)cc1)Nc1nc2ccc([C@](O)(C(=O)N3CC4CCC(C3)O4)C(F)(F)F)cc2s1] |
| 108 | 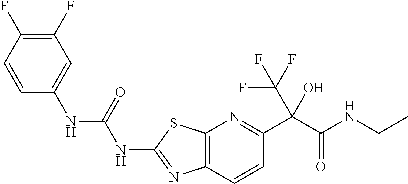 [SMILES: CCNC(=O)C(O)(c1ccc2nc(NC(=O)Nc3ccc(F)c(F)c3)sc2n1)C(F)(F)F] |
| 109 | 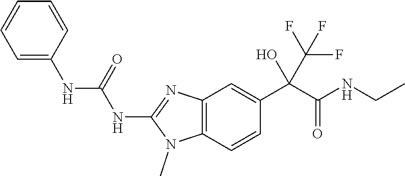 [SMILES: CCNC(=O)C(O)(c1ccc2c(c1)nc(NC(=O)Nc1ccccc1)n2C)C(F)(F)F] |
| 110 | 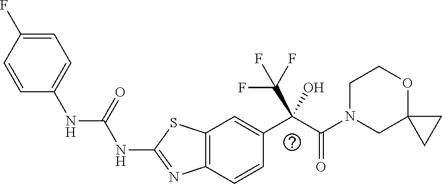 [SMILES: O=C(Nc1ccc(F)cc1)Nc1nc2ccc([C@](O)(C(=O)N3CCOC4(CC4)C3)C(F)(F)F)cc2s1] |
| 111 | 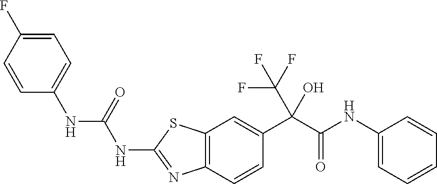 [SMILES: O=C(Nc1ccc(F)cc1)Nc1nc2ccc(C(O)(C(=O)Nc3ccccc3)C(F)(F)F)cc2s1] |
| 112 | 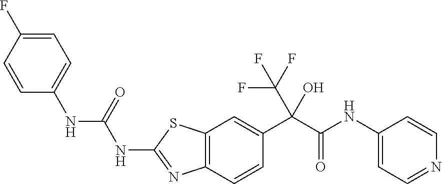 [SMILES: O=C(Nc1ccc(F)cc1)Nc1nc2ccc(C(O)(C(=O)Nc3ccncc3)C(F)(F)F)cc2s1] |
| 113 | 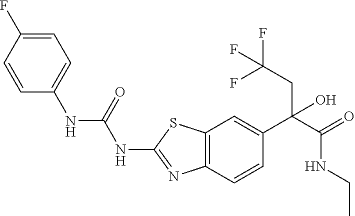 [SMILES: CCNC(=O)C(O)(CC(F)(F)F)c1ccc2nc(NC(=O)Nc3ccc(F)cc3)sc2c1] |
| 114 | 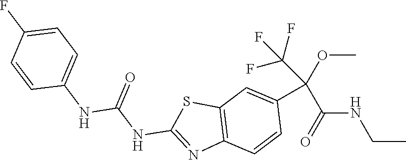 [SMILES: CCNC(=O)C(OC)(c1ccc2nc(NC(=O)Nc3ccc(F)cc3)sc2c1)C(F)(F)F] |
| 115 | 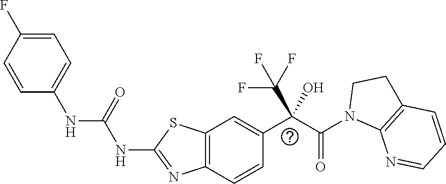 [SMILES: O=C(Nc1ccc(F)cc1)Nc1nc2ccc([C@](O)(C(=O)N3CCc4cccnc43)C(F)(F)F)cc2s1] |
| 116 | 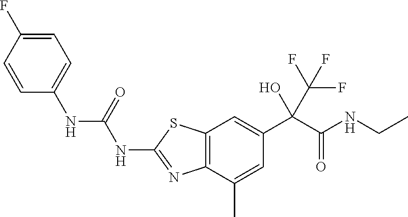 [SMILES: CCNC(=O)C(O)(c1cc(C)c2nc(NC(=O)Nc3ccc(F)cc3)sc2c1)C(F)(F)F] |
| 117 | 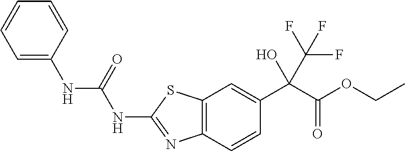 [SMILES: CCOC(=O)C(O)(c1ccc2nc(NC(=O)Nc3ccccc3)sc2c1)C(F)(F)F] |
| 118 | 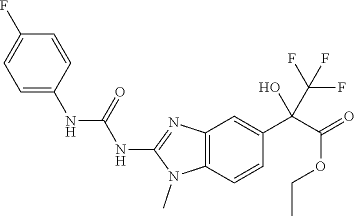 [SMILES: CCOC(=O)C(O)(c1ccc2c(c1)nc(NC(=O)Nc1ccc(F)cc1)n2C)C(F)(F)F] |
| 119 | 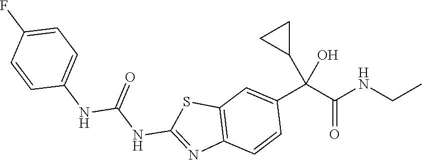 [SMILES: CCNC(=O)C(O)(c1ccc2nc(NC(=O)Nc3ccc(F)cc3)sc2c1)C1CC1] |
| 120 | 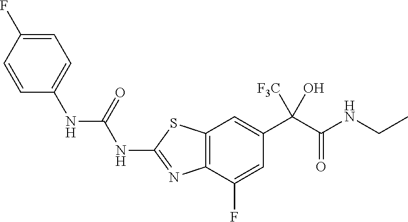 [SMILES: CCNC(=O)C(O)(c1cc(F)c2nc(NC(=O)Nc3ccc(F)cc3)sc2c1)C(F)(F)F] |
| 121 | 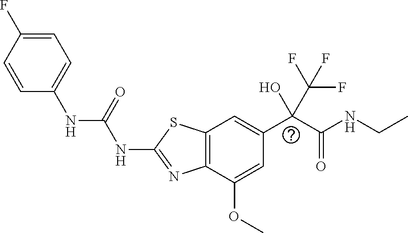 [SMILES: CCNC(=O)C(O)(c1cc(OC)c2nc(NC(=O)Nc3ccc(F)cc3)sc2c1)C(F)(F)F] |
| 122 | 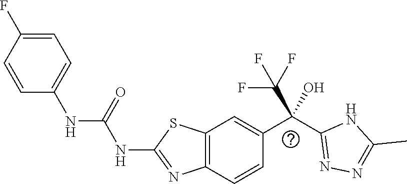 [SMILES: Cc1nnc([C@@](O)(c2ccc3nc(NC(=O)Nc4ccc(F)cc4)sc3c2)C(F)(F)F)[nH]1] |
| 123 | 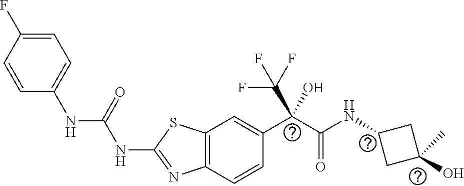 [SMILES: C[C@]1(O)C[C@H](NC(=O)[C@@](O)(c2ccc3nc(NC(=O)Nc4ccc(F)cc4)sc3c2)C(F)(F)F)C1] |
| 124 | 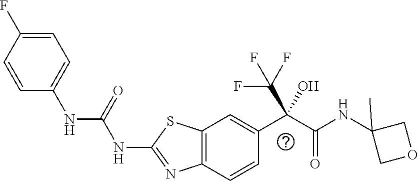 [SMILES: CC1(NC(=O)[C@@](O)(c2ccc3nc(NC(=O)Nc4ccc(F)cc4)sc3c2)C(F)(F)F)COC1] |
| 125 | 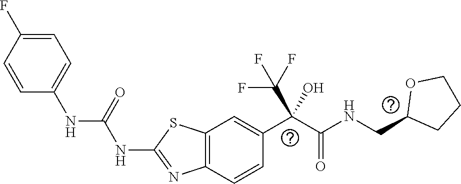 [SMILES: O=C(Nc1ccc(F)cc1)Nc1nc2ccc([C@](O)(C(=O)NC[C@@H]3CCCO3)C(F)(F)F)cc2s1] |
| 126 | 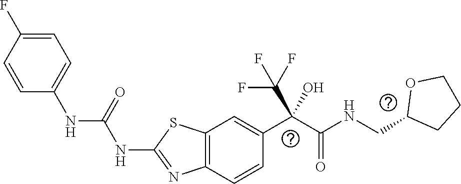 [SMILES: O=C(Nc1ccc(F)cc1)Nc1nc2ccc([C@](O)(C(=O)NC[C@H]3CCCO3)C(F)(F)F)cc2s1] |
| 127 | 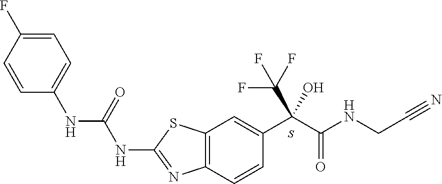 |
| 128 | 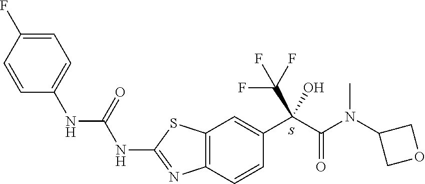 |
| 129 | 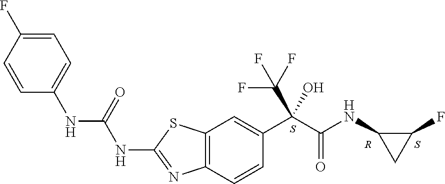 |
| 130 | 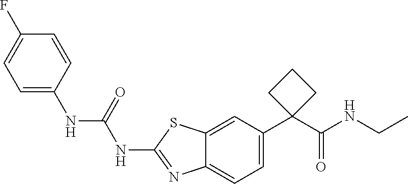 [SMILES: CCNC(=O)C1(c2ccc3nc(NC(=O)Nc4ccc(F)cc4)sc3c2)CCC1] |
| 131 | 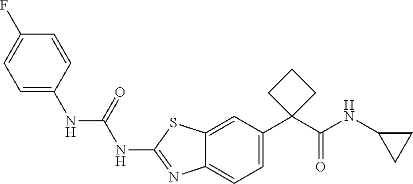 [SMILES: O=C(Nc1ccc(F)cc1)Nc1nc2ccc(C3(C(=O)NC4CC4)CCC3)cc2s1] |
| 132 | 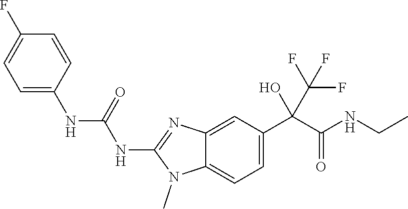 [SMILES: CCNC(=O)C(O)(c1ccc2c(c1)nc(NC(=O)Nc1ccc(F)cc1)n2C)C(F)(F)F] |
| 133 | 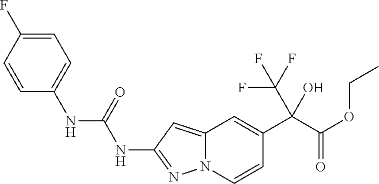 [SMILES: CCOC(=O)C(O)(c1ccn2nc(NC(=O)Nc3ccc(F)cc3)cc2c1)C(F)(F)F] |
| 134 | 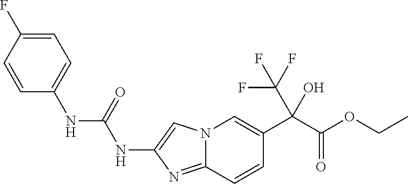 [SMILES: CCOC(=O)C(O)(c1ccc2nc(NC(=O)Nc3ccc(F)cc3)cn2c1)C(F)(F)F] |
| 135 | 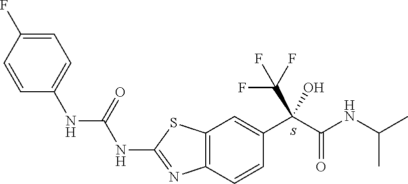 |
| 136 | 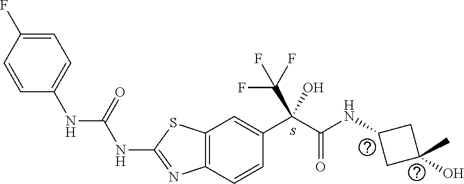 |
| 137 | 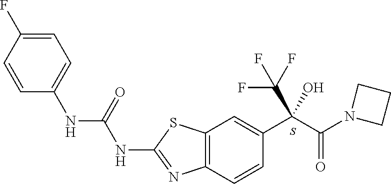 |
| 138 | 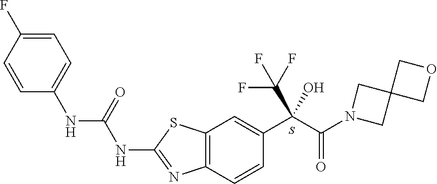 |
| 139 | 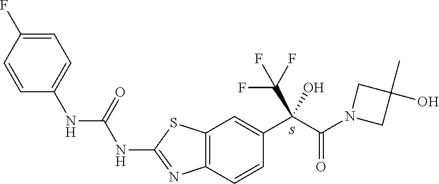 |
| 140 | 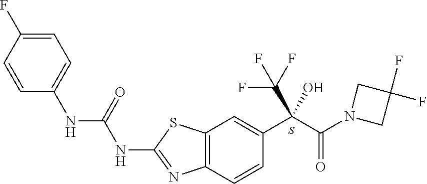 |
| 141 | 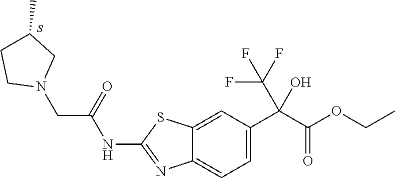 |
| 142 | 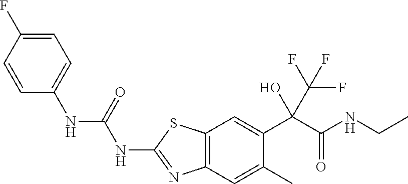 [SMILES: CCNC(=O)C(O)(c1cc2sc(NC(=O)Nc3ccc(F)cc3)nc2cc1C)C(F)(F)F] |
| 143 | 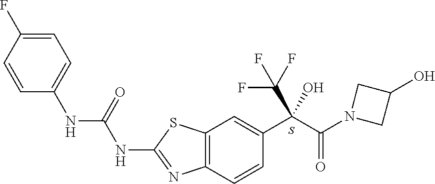 |
| 144 | 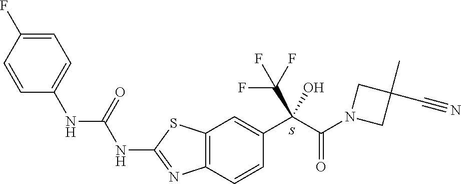 |
| 145 | 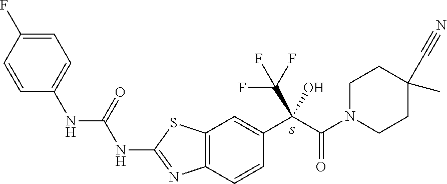 |
| 146 | 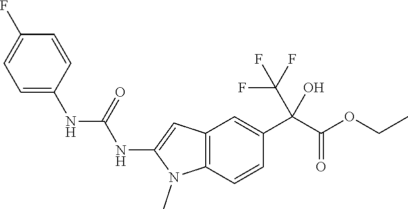 [SMILES: CCOC(=O)C(O)(c1ccc2c(c1)cc(NC(=O)Nc1ccc(F)cc1)n2C)C(F)(F)F] |
| 147 | 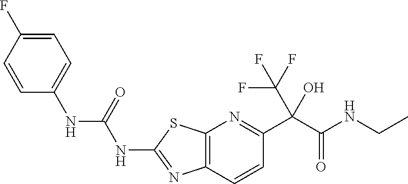 [SMILES: CCNC(=O)C(O)(c1ccc2nc(NC(=O)Nc3ccc(F)cc3)sc2n1)C(F)(F)F] |
| 148 | 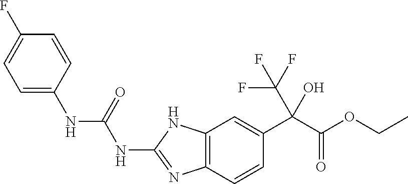 [SMILES: CCOC(=O)C(O)(c1ccc2nc(NC(=O)Nc3ccc(F)cc3)[nH]c2c1)C(F)(F)F] |
| 149 | 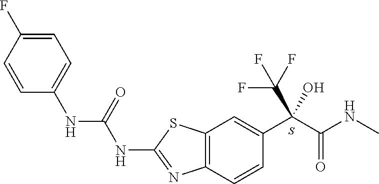 |
| 150 | 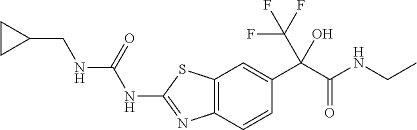 [SMILES: CCNC(=O)C(O)(c1ccc2nc(NC(=O)NCC3CC3)sc2c1)C(F)(F)F] |
| 151 | 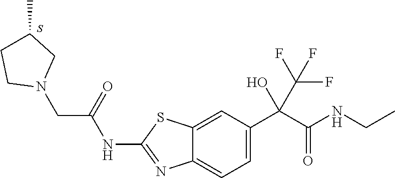 |
| 152 | 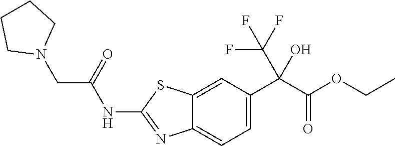 [SMILES: CCOC(=O)C(O)(c1ccc2nc(NC(=O)CN3CCCC3)sc2c1)C(F)(F)F] |
| 153 | 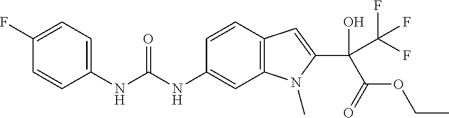 [SMILES: CCOC(=O)C(O)(c1cc2ccc(NC(=O)Nc3ccc(F)cc3)cc2n1C)C(F)(F)F] |
| 154 | 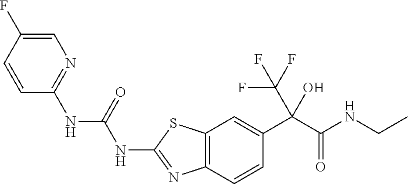 [SMILES: CCNC(=O)C(O)(c1ccc2nc(NC(=O)Nc3ccc(F)cn3)sc2c1)C(F)(F)F] |
| 155 | 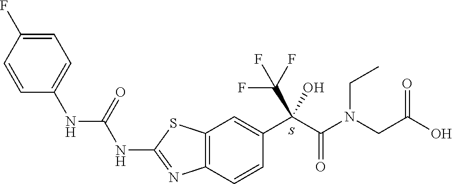 |
| 156 | 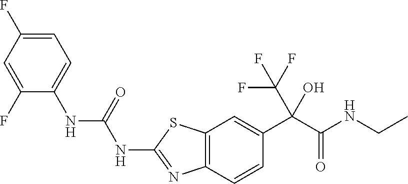 [SMILES: CCNC(=O)C(O)(c1ccc2nc(NC(=O)Nc3ccc(F)cc3F)sc2c1)C(F)(F)F] |
| 157 | 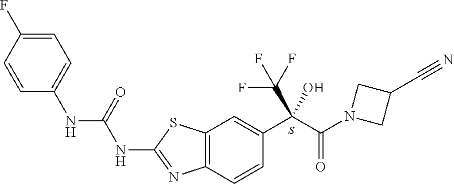 |
| 158 | 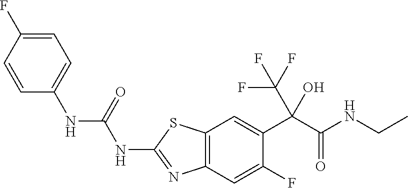 [SMILES: CCNC(=O)C(O)(c1cc2sc(NC(=O)Nc3ccc(F)cc3)nc2cc1F)C(F)(F)F] |
| 159 | 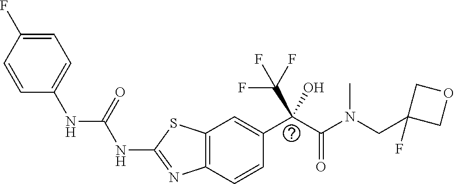 [SMILES: CN(CC1(F)COC1)C(=O)[C@@](O)(c1ccc2nc(NC(=O)Nc3ccc(F)cc3)sc2c1)C(F)(F)F] |
| 160 | 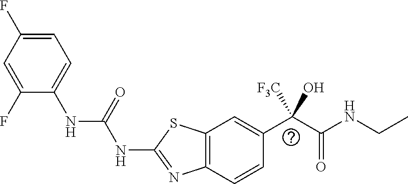 [SMILES: CCNC(=O)[C@](O)(c1ccc2nc(NC(=O)Nc3ccc(F)cc3F)sc2c1)C(F)(F)F] |
| 161 | 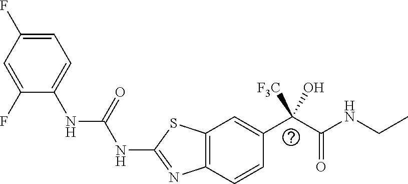 [SMILES: CCNC(=O)[C@@](O)(c1ccc2nc(NC(=O)Nc3ccc(F)cc3F)sc2c1)C(F)(F)F] |
| 162 | 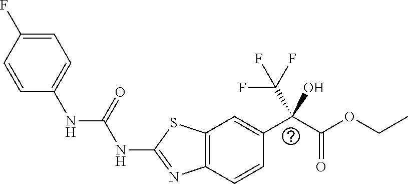 [SMILES: CCOC(=O)[C@](O)(c1ccc2nc(NC(=O)Nc3ccc(F)cc3)sc2c1)C(F)(F)F] |
| 164 | 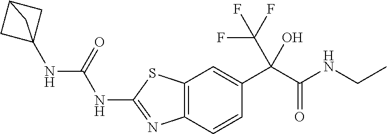 [SMILES: CCNC(=O)C(O)(c1ccc2nc(NC(=O)NC34CC(C3)C4)sc2c1)C(F)(F)F] |
| 165 | 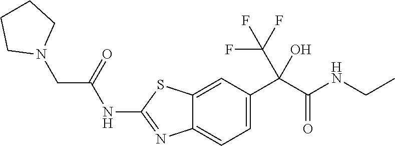 [SMILES: CCNC(=O)C(O)(c1ccc2nc(NC(=O)CN3CCCC3)sc2c1)C(F)(F)F] |
| 166 | 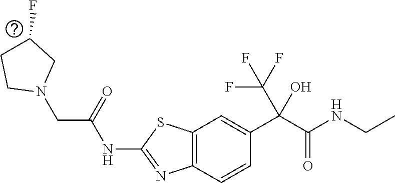 [SMILES: CCNC(=O)C(O)(c1ccc2nc(NC(=O)CN3CC[C@H](F)C3)sc2c1)C(F)(F)F] |
| 167 | 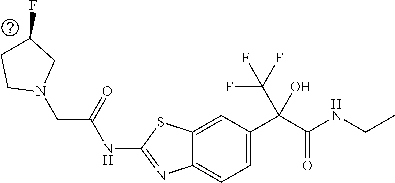 [SMILES: CCNC(=O)C(O)(c1ccc2nc(NC(=O)CN3CC[C@@H](F)C3)sc2c1)C(F)(F)F] |
| 168 | 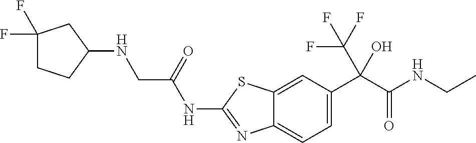 [SMILES: CCNC(=O)C(O)(c1ccc2nc(NC(=O)CNC3CCC(F)(F)C3)sc2c1)C(F)(F)F] |
| 169 | 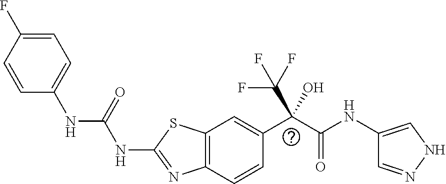 [SMILES: O=C(Nc1ccc(F)cc1)Nc1nc2ccc([C@](O)(C(=O)Nc3cn[nH]c3)C(F)(F)F)cc2s1] |
| 170 | 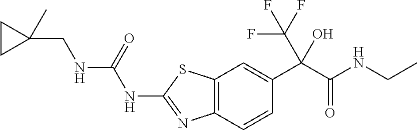 [SMILES: CCNC(=O)C(O)(c1ccc2nc(NC(=O)NCC3(C)CC3)sc2c1)C(F)(F)F] |
| 171 | 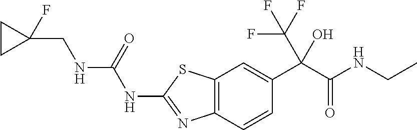 [SMILES: CCNC(=O)C(O)(c1ccc2nc(NC(=O)NCC3(F)CC3)sc2c1)C(F)(F)F] |
| 172 | 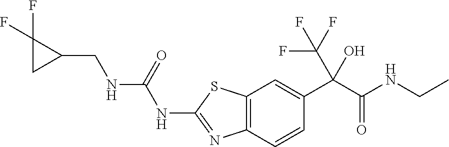 [SMILES: CCNC(=O)C(O)(c1ccc2nc(NC(=O)NCC3CC3(F)F)sc2c1)C(F)(F)F] |
| 173 | 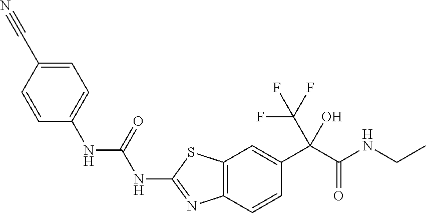 [SMILES: CCNC(=O)C(O)(c1ccc2nc(NC(=O)Nc3ccc(C#N)cc3)sc2c1)C(F)(F)F] |
| 174 | 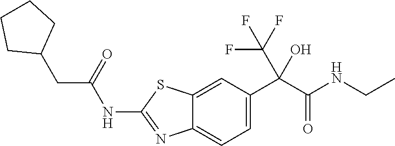 [SMILES: CCNC(=O)C(O)(c1ccc2nc(NC(=O)CC3CCCC3)sc2c1)C(F)(F)F] |
| 175 | 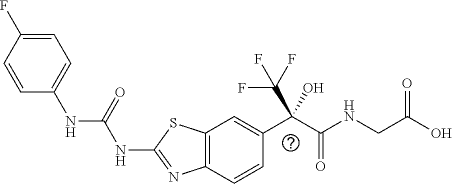 [SMILES: O=C(O)CNC(=O)[C@@](O)(c1ccc2nc(NC(=O)Nc3ccc(F)cc3)sc2c1)C(F)(F)F] |
| 176 | 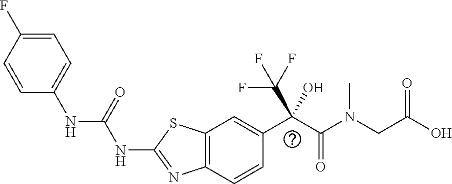 [SMILES: CN(CC(=O)O)C(=O)[C@@](O)(c1ccc2nc(NC(=O)Nc3ccc(F)cc3)sc2c1)C(F)(F)F] |
| 177 | 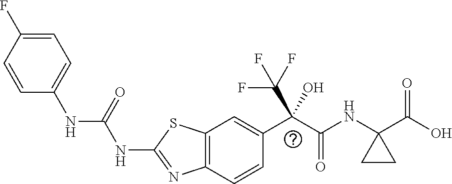 [SMILES: O=C(Nc1ccc(F)cc1)Nc1nc2ccc([C@](O)(C(=O)NC3(C(=O)O)CC3)C(F)(F)F)cc2s1] |
| 178 | 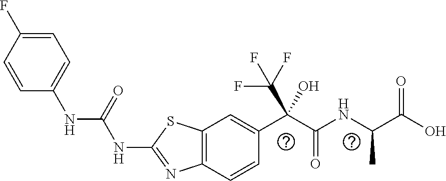 [SMILES: C[C@@H](NC(=O)[C@@](O)(c1ccc2nc(NC(=O)Nc3ccc(F)cc3)sc2c1)C(F)(F)F)C(=O)O] |
| 179 | 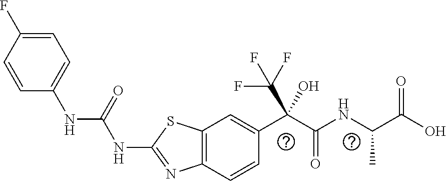 [SMILES: C[C@H](NC(=O)[C@@](O)(c1ccc2nc(NC(=O)Nc3ccc(F)cc3)sc2c1)C(F)(F)F)C(=O)O] |
| 180 | 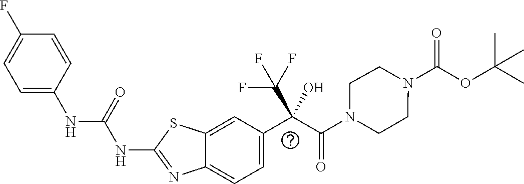 [SMILES: CC(C)(C)OC(=O)N1CCN(C(=O)[C@@](O)(c2ccc3nc(NC(=O)Nc4ccc(F)cc4)sc3c2)C(F)(F)F)CC1] |
| 181 | 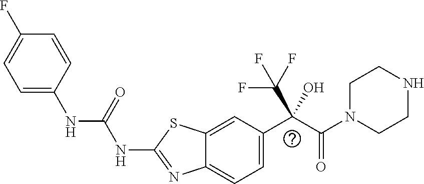 [SMILES: O=C(Nc1ccc(F)cc1)Nc1nc2ccc([C@](O)(C(=O)N3CCNCC3)C(F)(F)F)cc2s1] |
| 182 | 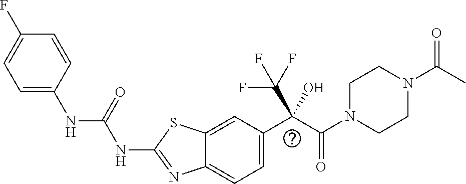 [SMILES: CC(=O)N1CCN(C(=O)[C@@](O)(c2ccc3nc(NC(=O)Nc4ccc(F)cc4)sc3c2)C(F)(F)F)CC1] |
| 183 | 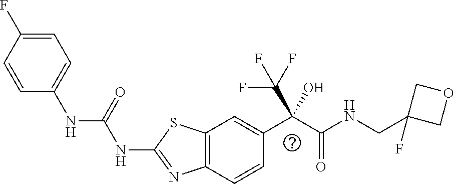 [SMILES: O=C(Nc1ccc(F)cc1)Nc1nc2ccc([C@](O)(C(=O)NCC3(F)COC3)C(F)(F)F)cc2s1] [symbol] |
| 184 | 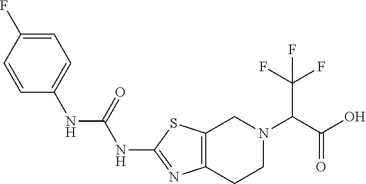 [SMILES: O=C(Nc1ccc(F)cc1)Nc1nc2c(s1)CN(C(C(=O)O)C(F)(F)F)CC2] |
| 185 | 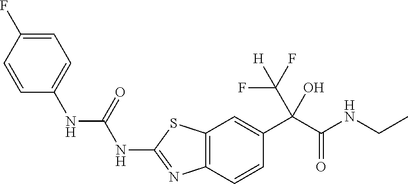 [SMILES: CCNC(=O)C(O)(c1ccc2nc(NC(=O)Nc3ccc(F)cc3)sc2c1)C(F)F] |
| 186 | 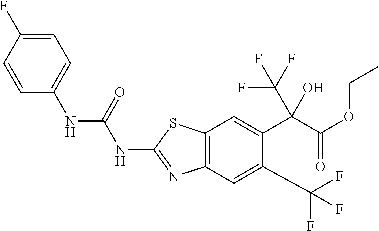 [SMILES: CCOC(=O)C(O)(c1cc2sc(NC(=O)Nc3ccc(F)cc3)nc2cc1C(F)(F)F)C(F)(F)F] |
| 187 | 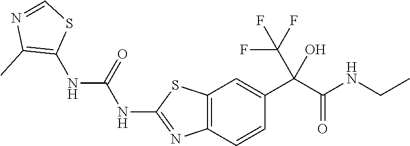 [SMILES: CCNC(=O)C(O)(c1ccc2nc(NC(=O)Nc3scnc3C)sc2c1)C(F)(F)F] |
| 188 | 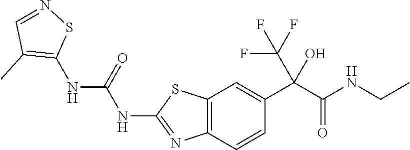 [SMILES: CCNC(=O)C(O)(c1ccc2nc(NC(=O)Nc3sncc3C)sc2c1)C(F)(F)F] |
| 189 | 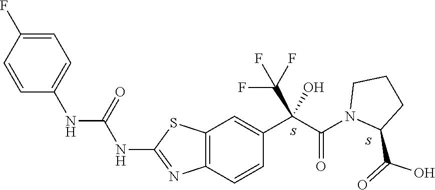 |
| 190 | 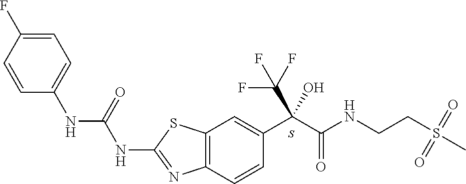 |
| 191 | 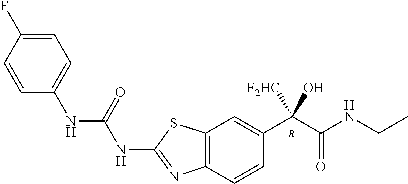 |
| 192 | 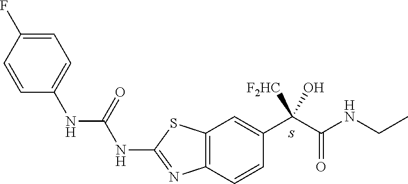 |
| 193 | 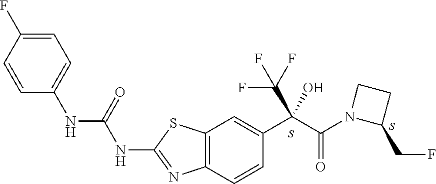 |
| 194 | 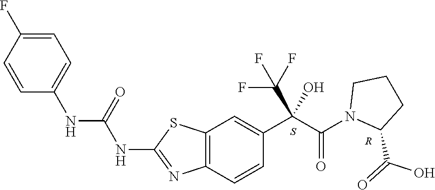 |
| 195 | 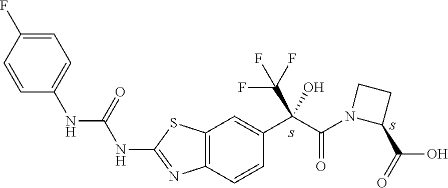 |
| 196 | 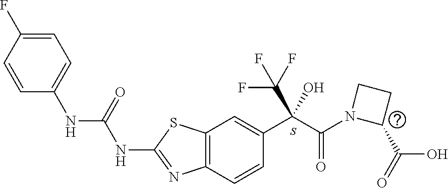 |
| 197 | 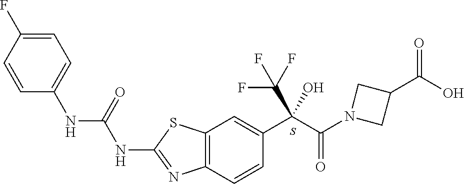 |
| 198 | 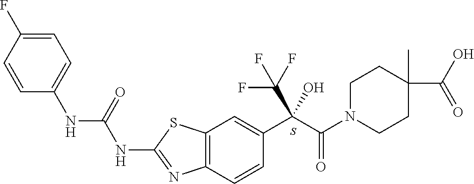 |
| 199 | 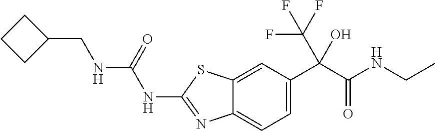 [SMILES: CCNC(=O)C(O)(c1ccc2nc(NC(=O)NCC3CCC3)sc2c1)C(F)(F)F] |
| 200 | 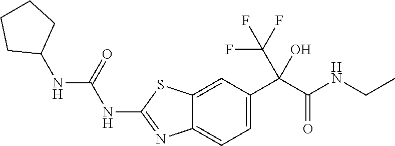 [SMILES: CCNC(=O)C(O)(c1ccc2nc(NC(=O)NC3CCCC3)sc2c1)C(F)(F)F] |
| 201 | 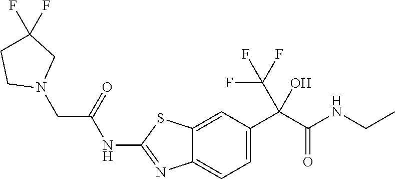 [SMILES: CCNC(=O)C(O)(c1ccc2nc(NC(=O)CN3CCC(F)(F)C3)sc2c1)C(F)(F)F] |
| 202 | 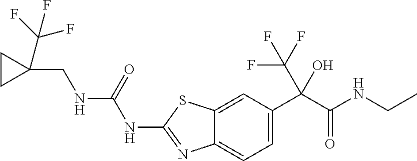 [SMILES: CCNC(=O)C(O)(c1ccc2nc(NC(=O)NCC3(C(F)(F)F)CC3)sc2c1)C(F)(F)F] |
| 203 | 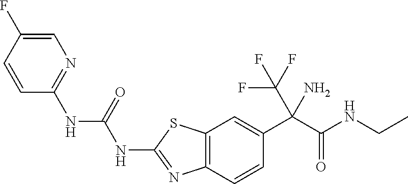 [SMILES: CCNC(=O)C(N)(c1ccc2nc(NC(=O)Nc3ccc(F)cn3)sc2c1)C(F)(F)F] |
| 204 | 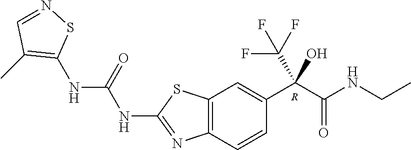 |
| 205 | 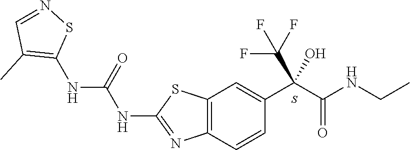 |
| 206 | 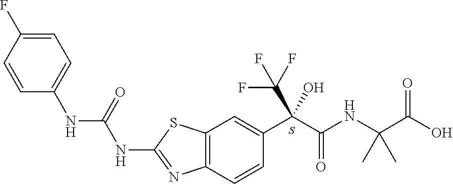 |
| 207 | 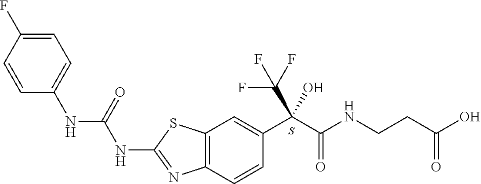 |
| 208 | 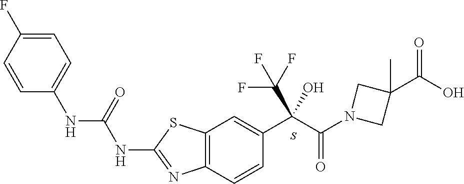 |
| 209 | 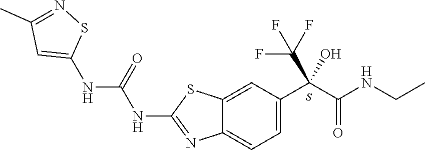 |
| 210 | 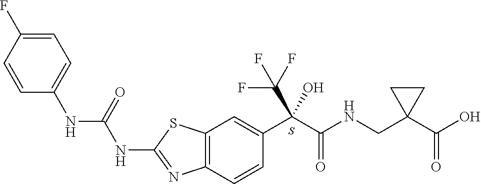 |
| 211 | 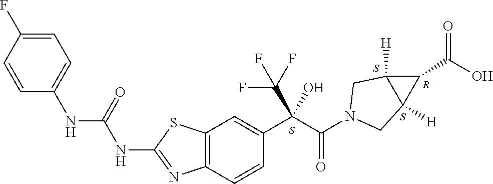 |

[symbol]

indicates data missing or illegible when filed

or a pharmaceutically acceptable salt thereof.

In still other embodiments, the compound has a structure selected from the following table:

| # | Structure |
|---|---|
| 212 | 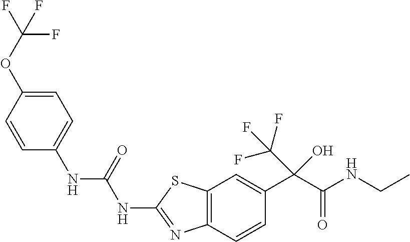 [SMILES: CCNC(=O)C(O)(c1ccc2nc(NC(=O)Nc3ccc(OC(F)(F)F)cc3)sc2c1)C(F)(F)F] |
| 213 | 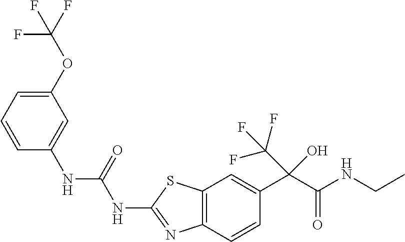 [SMILES: CCNC(=O)C(O)(c1ccc2nc(NC(=O)Nc3cccc(OC(F)(F)F)c3)sc2c1)C(F)(F)F] |
| 214 | 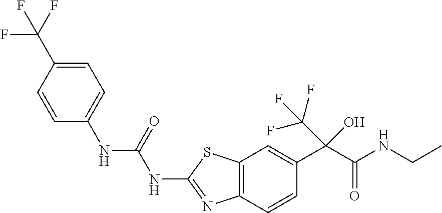 [SMILES: CCNC(=O)C(O)(c1ccc2nc(NC(=O)Nc3ccc(C(F)(F)F)cc3)sc2c1)C(F)(F)F] |
| 215 | 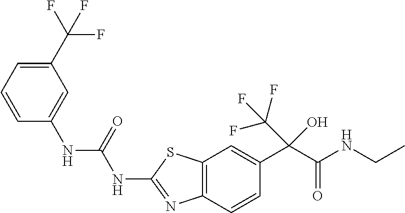 [SMILES: CCNC(=O)C(O)(c1ccc2nc(NC(=O)Nc3cccc(C(F)(F)F)c3)sc2c1)C(F)(F)F] |
| 216 | 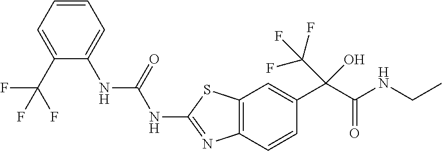 [SMILES: CCNC(=O)C(O)(c1ccc2nc(NC(=O)Nc3ccccc3C(F)(F)F)sc2c1)C(F)(F)F] |
| 217 | 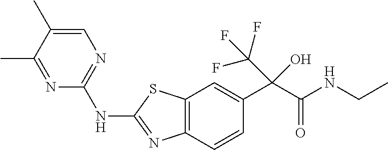 [SMILES: CCNC(=O)C(O)(c1ccc2nc(Nc3ncc(C)c(C)n3)sc2c1)C(F)(F)F] |
| 218 | 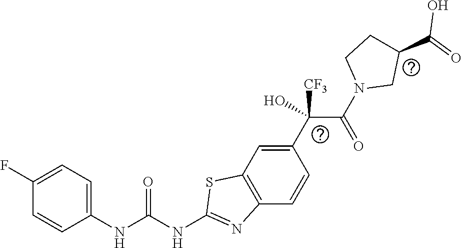 [SMILES: O=C(Nc1ccc(F)cc1)Nc1nc2ccc([C@](O)(C(=O)N3CC[C@@H](C(=O)O)C3)C(F)(F)F)cc2s1] |
| 219 | 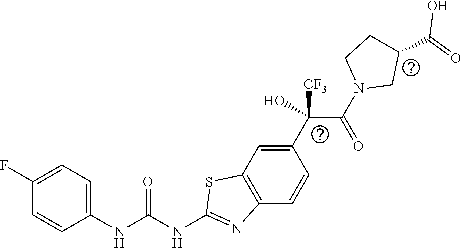 [SMILES: O=C(Nc1ccc(F)cc1)Nc1nc2ccc([C@](O)(C(=O)N3CC[C@H](C(=O)O)C3)C(F)(F)F)cc2s1] |
| 220 | 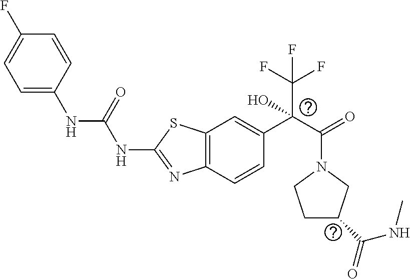 [SMILES: CNC(=O)[C@@H]1CCN(C(=O)[C@@](O)(c2ccc3nc(NC(=O)Nc4ccc(F)cc4)sc3c2)C(F)(F)F)C1] |
| 221 | 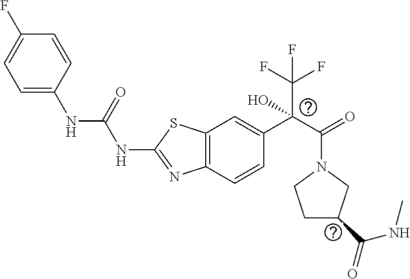 [SMILES: CNC(=O)[C@H]1CCN(C(=O)[C@@](O)(c2ccc3nc(NC(=O)Nc4ccc(F)cc4)sc3c2)C(F)(F)F)C1] |
| 222 | 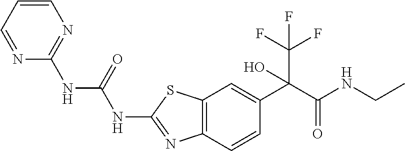 [SMILES: CCNC(=O)C(O)(c1ccc2nc(NC(=O)Nc3ncccn3)sc2c1)C(F)(F)F] |
| 223 | 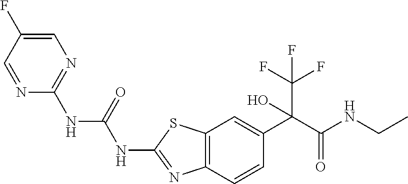 [SMILES: CCNC(=O)C(O)(c1ccc2nc(NC(=O)Nc3ncc(F)cn3)sc2c1)C(F)(F)F] |
| 224 | 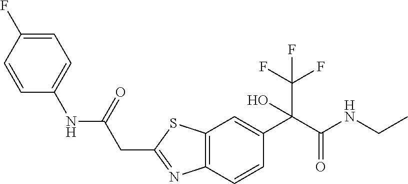 [SMILES: CCNC(=O)C(O)(c1ccc2nc(CC(=O)Nc3ccc(F)cc3)sc2c1)C(F)(F)F] |
| 225 | 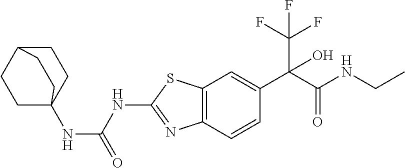 [SMILES: CCNC(=O)C(O)(c1ccc2nc(NC(=O)NC34CCC(CC3)CC4)sc2c1)C(F)(F)F] |
| 226 | 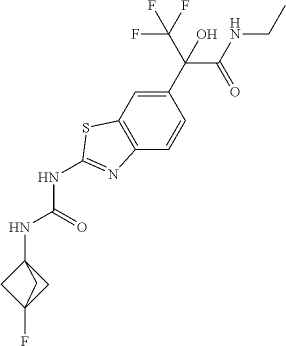 [SMILES: CCNC(=O)C(O)(c1ccc2nc(NC(=O)NC34CC(F)(C3)C4)sc2c1)C(F)(F)F] |
| 227 | 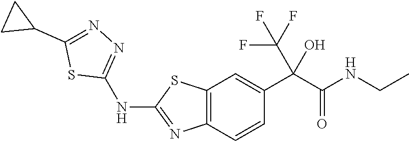 [SMILES: CCNC(=O)C(O)(c1ccc2nc(Nc3nnc(C4CC4)s3)sc2c1)C(F)(F)F] |
| 228 | 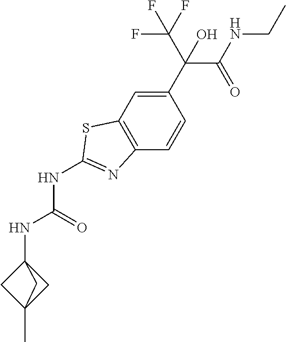 [SMILES: CCNC(=O)C(O)(c1ccc2nc(NC(=O)NC34CC(C)(C3)C4)sc2c1)C(F)(F)F] |
| 229 | 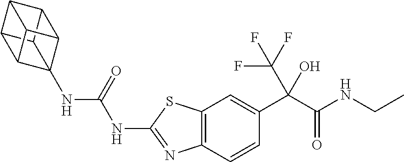 [SMILES: CCNC(=O)C(O)(c1ccc2nc(NC(=O)NC34C5C6C7C5C3C7C64)sc2c1)C(F)(F)F] |
| 230 | 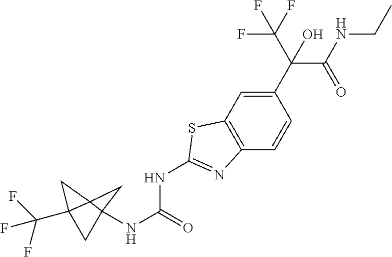 [SMILES: CCNC(=O)C(O)(c1ccc2nc(NC(=O)NC34CC5(C(F)(F)F)CC35C4)sc2c1)C(F)(F)F] |
| 231 | 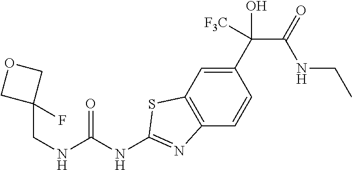 [SMILES: CCNC(=O)C(O)(c1ccc2nc(NC(=O)NCC3(F)COC3)sc2c1)C(F)(F)F] |
| 232 | 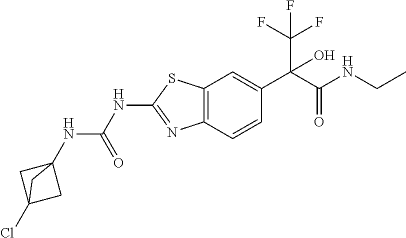 [SMILES: CCNC(=O)C(O)(c1ccc2nc(NC(=O)NC34CC(Cl)(C3)C4)sc2c1)C(F)(F)F] |
| 233 | 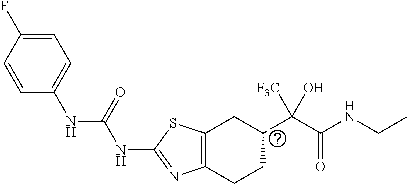 [SMILES: CCNC(=O)C(O)([C@@H]1CCc2nc(NC(=O)Nc3ccc(F)cc3)sc2C1)C(F)(F)F] |
| 234 | 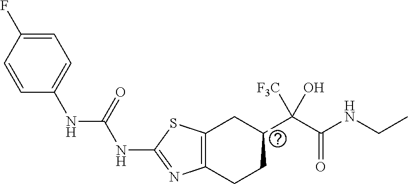 [SMILES: CCNC(=O)C(O)([C@H]1CCc2nc(NC(=O)Nc3ccc(F)cc3)sc2C1)C(F)(F)F] |
| 235 | 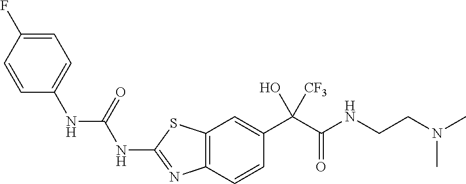 [SMILES: CN(C)CCNC(=O)C(O)(c1ccc2nc(NC(=O)Nc3ccc(F)cc3)sc2c1)C(F)(F)F] |
| 236 | 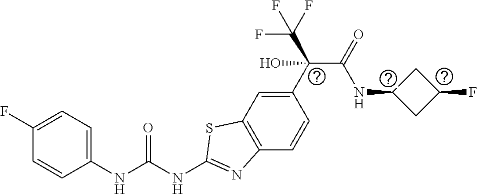 [SMILES: O=C(Nc1ccc(F)cc1)Nc1nc2ccc([C@](O)(C(=O)N[C@H]3C[C@@H](F)C3)C(F)(F)F)cc2s1] |
| 237 | 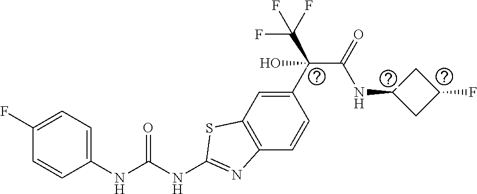 [SMILES: O=C(Nc1ccc(F)cc1)Nc1nc2ccc([C@](O)(C(=O)N[C@H]3C[C@H](F)C3)C(F)(F)F)cc2s1] |
| 238 | 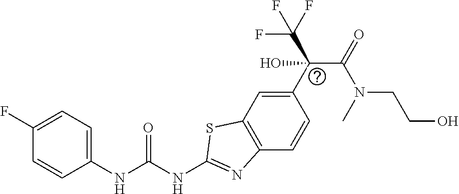 [SMILES: CN(CCO)C(=O)[C@@](O)(c1ccc2nc(NC(=O)Nc3ccc(F)cc3)sc2c1)C(F)(F)F] |
| 239 | 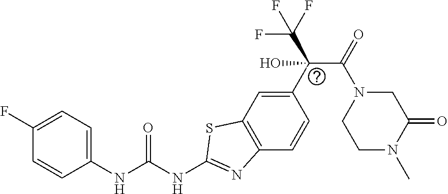 [SMILES: CN1CCN(C(=O)[C@@](O)(c2ccc3nc(NC(=O)Nc4ccc(F)cc4)sc3c2)C(F)(F)F)CC1=O] |
| 240 | 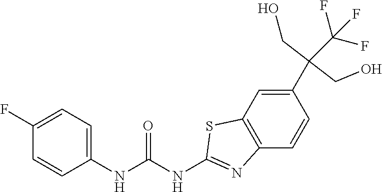 [SMILES: O=C(Nc1ccc(F)cc1)Nc1nc2ccc(C(CO)(CO)C(F)(F)F)cc2s1] |
| 241 | 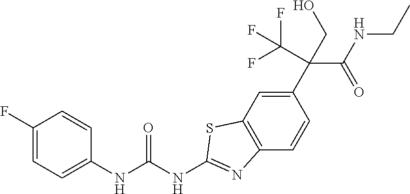 [SMILES: CCNC(=O)C(CO)(c1ccc2nc(NC(=O)Nc3ccc(F)cc3)sc2c1)C(F)(F)F] |
| 242 | 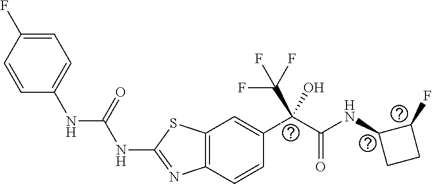 [SMILES: O=C(Nc1ccc(F)cc1)Nc1nc2ccc([C@](O)(C(=O)N[C@@H]3CC[C@@H]3F)C(F)(F)F)cc2s1] |
| 243 | 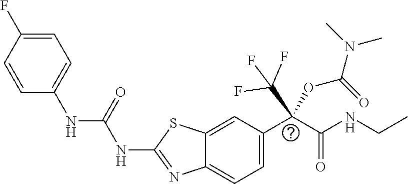 [SMILES: CCNC(=O)[C@@](OC(=O)N(C)C)(c1ccc2nc(NC(=O)Nc3ccc(F)cc3)sc2c1)C(F)(F)F] |
| 244 | 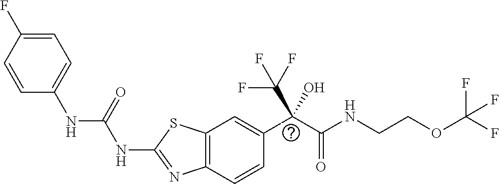 [SMILES: O=C(Nc1ccc(F)cc1)Nc1nc2ccc([C@](O)(C(=O)NCCOC(F)(F)F)C(F)(F)F)cc2s1] |
| 245 | 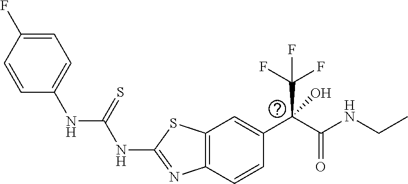 [SMILES: CCNC(=O)[C@@](O)(c1ccc2nc(NC(=S)Nc3ccc(F)cc3)sc2c1)C(F)(F)F] |
| 246 | 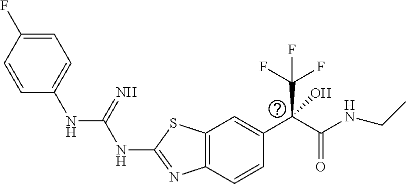 [SMILES: CCNC(=O)[C@@](O)(c1ccc2nc(NC(=N)Nc3ccc(F)cc3)sc2c1)C(F)(F)F] |
| 247 | 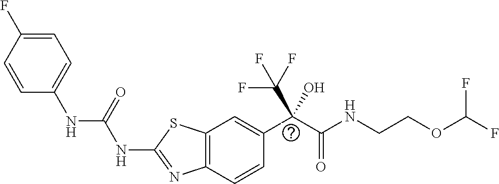 [SMILES: O=C(Nc1ccc(F)cc1)Nc1nc2ccc([C@](O)(C(=O)NCCOC(F)F)C(F)(F)F)cc2s1] |
| 248 | 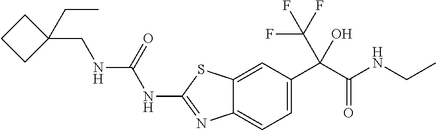 [SMILES: CCNC(=O)C(O)(c1ccc2nc(NC(=O)NCC3(CC)CCC3)sc2c1)C(F)(F)F] |
| 249 | 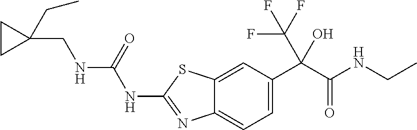 [SMILES: CCNC(=O)C(O)(c1ccc2nc(NC(=O)NCC3(CC)CC3)sc2c1)C(F)(F)F] |
| 250 | 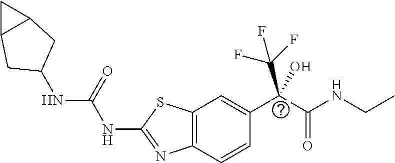 [SMILES: CCNC(=O)[C@@](O)(c1ccc2nc(NC(=O)NC3CC4CC4C3)sc2c1)C(F)(F)F] |
| 251 | 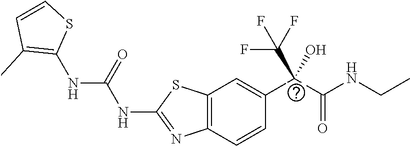 [SMILES: CCNC(=O)[C@@](O)(c1ccc2nc(NC(=O)Nc3sccc3C)sc2c1)C(F)(F)F] |
| 252 | 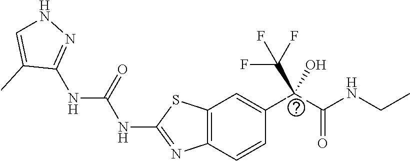 [SMILES: CCNC(=O)[C@@](O)(c1ccc2nc(NC(=O)Nc3n[nH]cc3C)sc2c1)C(F)(F)F] |
| 253 | 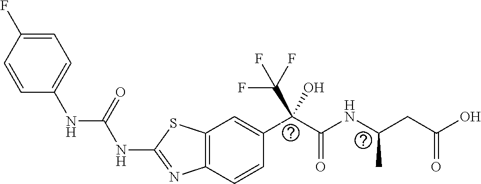 [SMILES: C[C@H](CC(=O)O)NC(=O)[C@@](O)(c1ccc2nc(NC(=O)Nc3ccc(F)cc3)sc2c1)C(F)(F)F] |
| 254 | 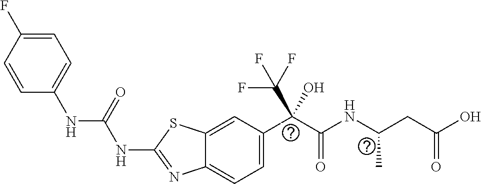 [SMILES: C[C@@H](CC(=O)O)NC(=O)[C@@](O)(c1ccc2nc(NC(=O)Nc3ccc(F)cc3)sc2c1)C(F)(F)F] |
| 255 | 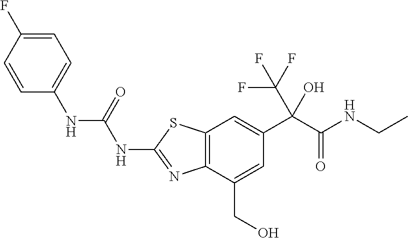 [SMILES: CCNC(=O)C(O)(c1cc(CO)c2nc(NC(=O)Nc3ccc(F)cc3)sc2c1)C(F)(F)F] |
| 256 | 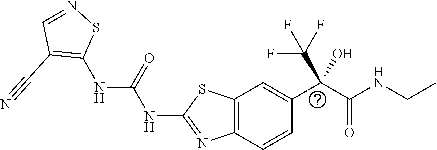 [SMILES: CCNC(=O)[C@@](O)(c1ccc2nc(NC(=O)Nc3sncc3C#N)sc2c1)C(F)(F)F] |
| 257 | 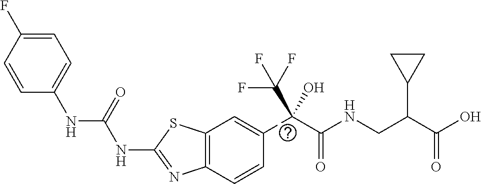 [SMILES: O=C(Nc1ccc(F)cc1)Nc1nc2ccc([C@](O)(C(=O)NCC(C(=O)O)C3CC3)C(F)(F)F)cc2s1] |
| 258 | 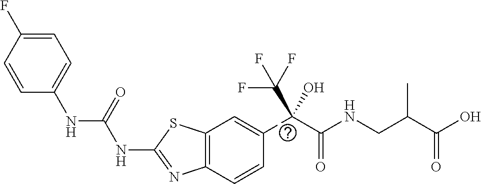 [SMILES: CC(CNC(=O)[C@@](O)(c1ccc2nc(NC(=O)Nc3ccc(F)cc3)sc2c1)C(F)(F)F)C(=O)O] |
| 259 | 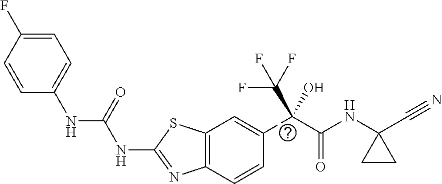 [SMILES: N#CC1(NC(=O)[C@@](O)(c2ccc3nc(NC(=O)Nc4ccc(F)cc4)sc3c2)C(F)(F)F)CC1] |
| 260 | 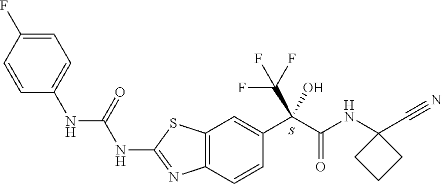 |
| 261 | 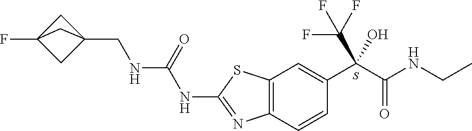 |
| 262 | 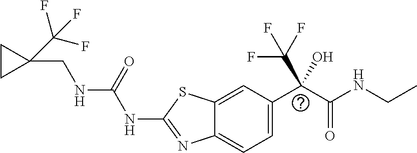 [SMILES: CCNC(=O)[C@@](O)(c1ccc2nc(NC(=O)NCC3(C(F)(F)F)CC3)sc2c1)C(F)(F)F] |
| 263 | 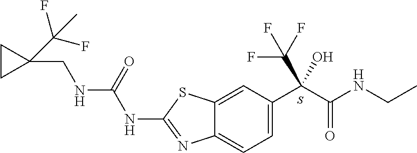 |
| 264 | 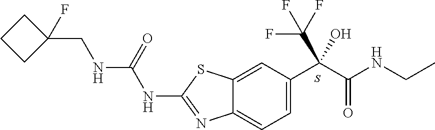 |
| 265 | 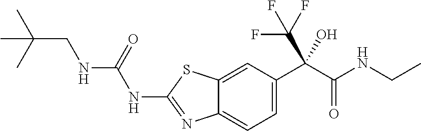 [SMILES: CCNC(=O)[C@@](O)(c1ccc2nc(NC(=O)NCC(C)(C)C)sc2c1)C(F)(F)F] |
| 266 | 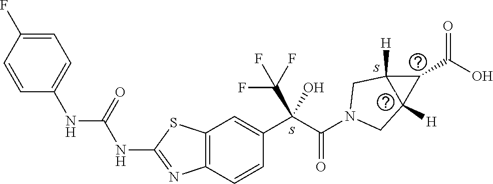 |
| 267 | 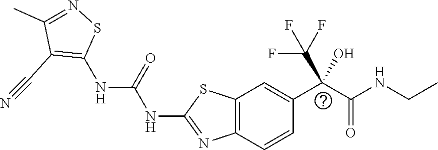 [SMILES: CCNC(=O)[C@@](O)(c1ccc2nc(NC(=O)Nc3snc(C)c3C#N)sc2c1)C(F)(F)F] |
| 268 | 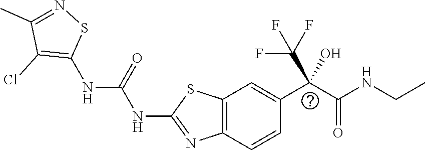 [SMILES: CCNC(=O)[C@@](O)(c1ccc2nc(NC(=O)Nc3snc(C)c3Cl)sc2c1)C(F)(F)F] |
| 269 | 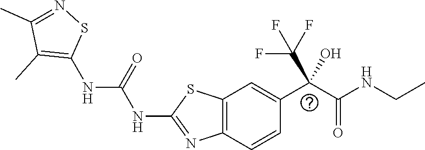 [SMILES: CCNC(=O)[C@@](O)(c1ccc2nc(NC(=O)Nc3snc(C)c3C)sc2c1)C(F)(F)F] |
| 270 | 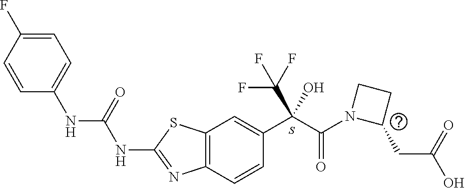 |
| 271 | 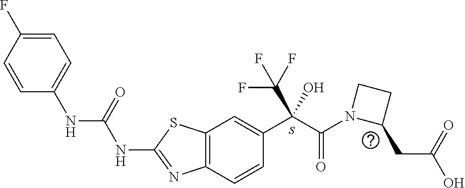 |
| 272 | 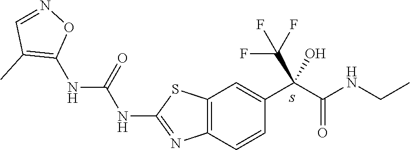 |
| 273 | 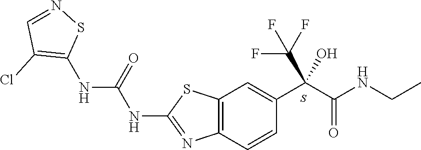 |
| 274 | 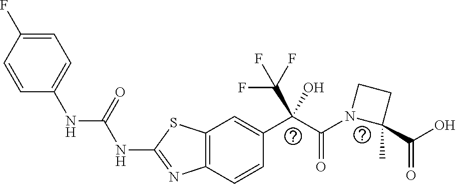 [SMILES: C[C@@]1(C(=O)O)CCN1C(=O)[C@@](O)(c1ccc2nc(NC(=O)Nc3ccc(F)cc3)sc2c1)C(F)(F)F] |
| 275 | 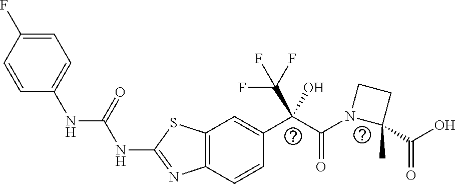 [SMILES: C[C@]1(C(=O)O)CCN1C(=O)[C@@](O)(c1ccc2nc(NC(=O)Nc3ccc(F)cc3)sc2c1)C(F)(F)F] |
| 276 | 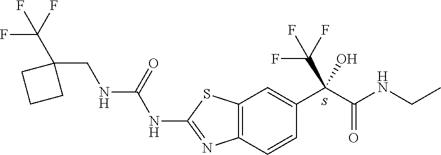 |
| 277 | 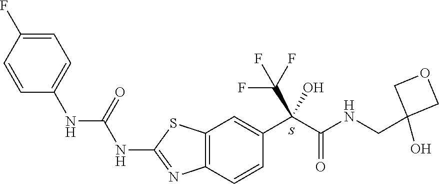 |
| 278 | 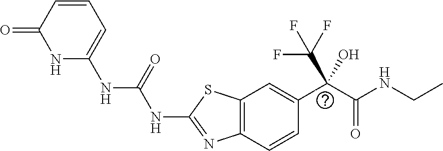 [SMILES: CCNC(=O)[C@@](O)(c1ccc2nc(NC(=O)Nc3cccc(=O)[nH]3)sc2c1)C(F)(F)F] |
| 279 | 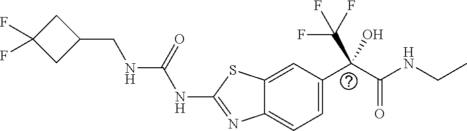 [SMILES: CCNC(=O)[C@@](O)(c1ccc2nc(NC(=O)NCC3CC(F)(F)C3)sc2c1)C(F)(F)F] |
| 280 | 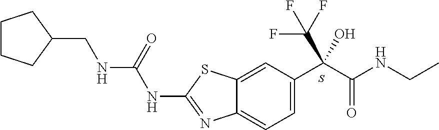 |
| 281 | 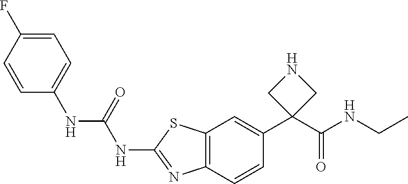 [SMILES: CCNC(=O)C1(c2ccc3nc(NC(=O)Nc4ccc(F)cc4)sc3c2)CNC1] |
| 282 | 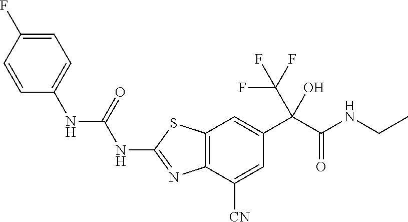 [SMILES: CCNC(=O)C(O)(c1cc(C#N)c2nc(NC(=O)Nc3ccc(F)cc3)sc2c1)C(F)(F)F] |
| 283 | 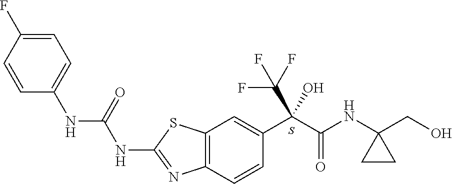 |
| 284 | 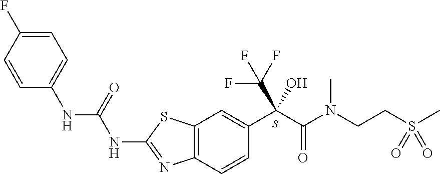 |
| 285 | 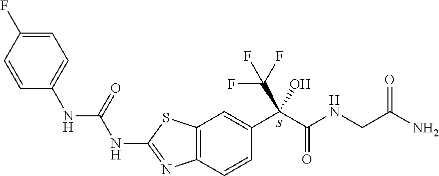 |
| 286 | 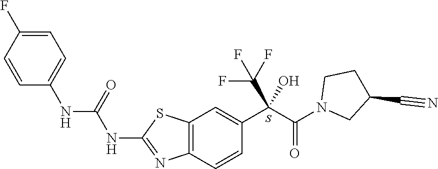 |
| 287 | 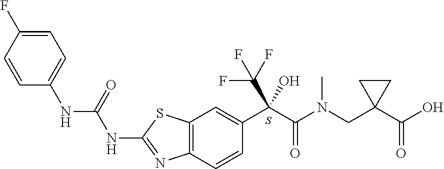 |
| 288 | 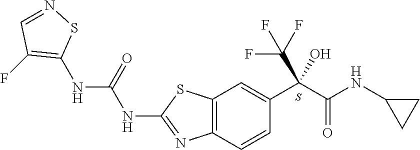 |
| 289 | 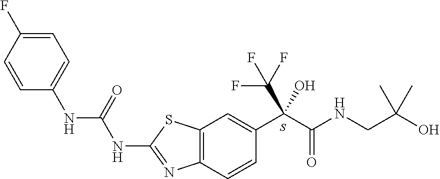 |
| 290 | 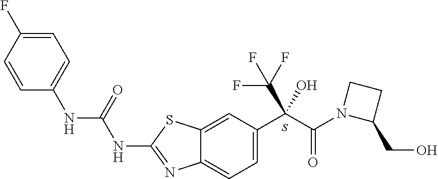 |
| 291 | 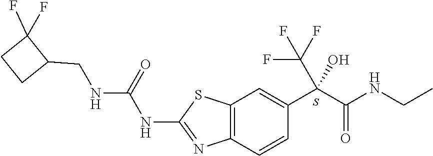 |
| 292 | 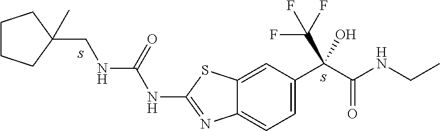 |
| 293 | 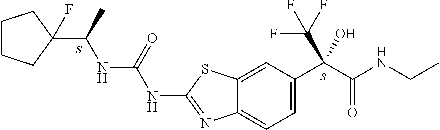 |
| 294 | 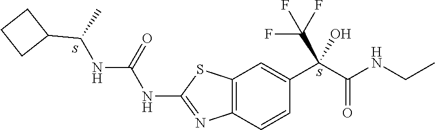 |
| 295 | 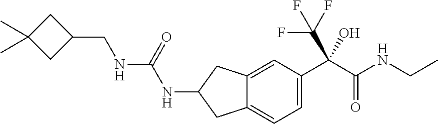 [SMILES: CCNC(=O)[C@@](O)(c1ccc2c(c1)CC(NC(=O)NCC1CC(C)(C)C1)C2)C(F)(F)F] |
| 296 | 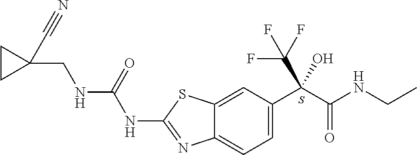 |
| 297 | 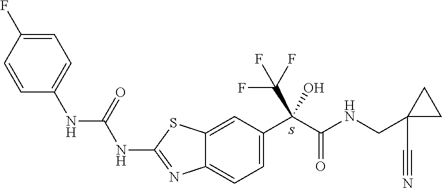 |
| 298 | 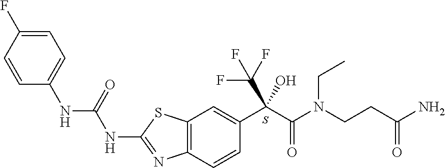 |
| 299 | 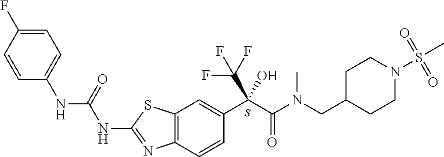 |
| 300 | 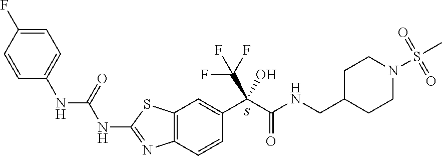 |
| 301 | 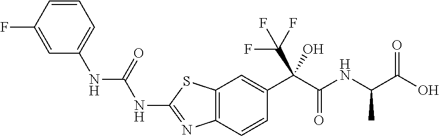 [SMILES: C[C@@H](NC(=O)[C@@](O)(c1ccc2nc(NC(=O)Nc3cccc(F)c3)sc2c1)C(F)(F)F)C(=O)O] |
| 302 | 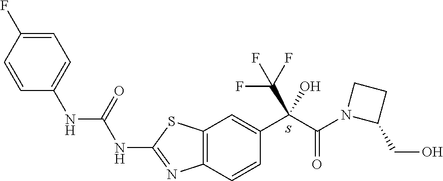 |
| 303 | 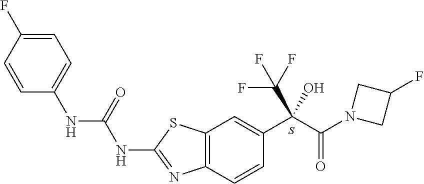 |
| 304 | 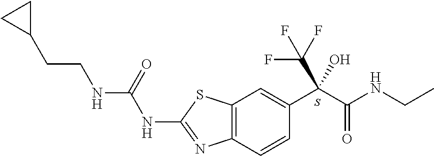 |
| 305 | 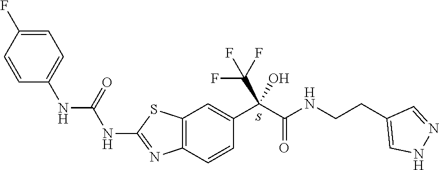 |
| 306 | 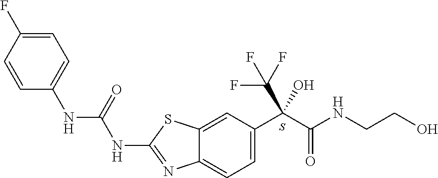 |
| 307 | 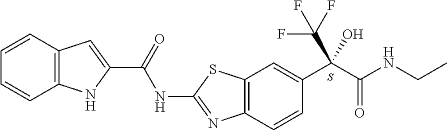 |
| 308 | 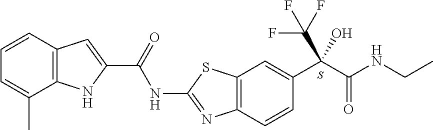 |
| 309 | 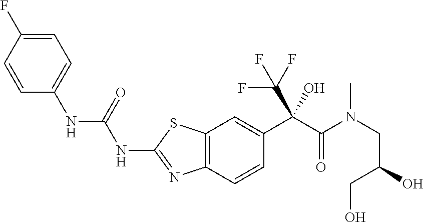 [SMILES: CN(C[C@@H](O)CO)C(=O)[C@@](O)(c1ccc2nc(NC(=O)Nc3ccc(F)cc3)sc2c1)C(F)(F)F] |
| 310 | 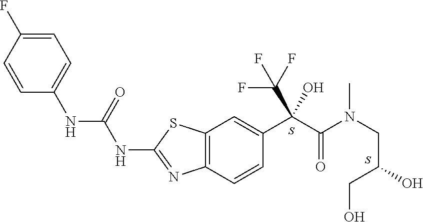 |
| 311 | 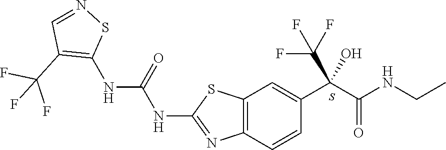 |
| 312 | 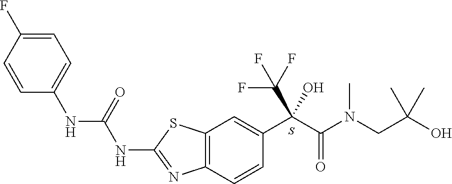 |
| 313 | 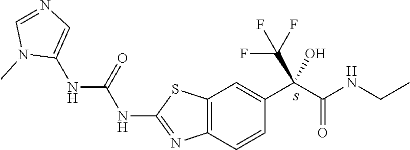 |
| 314 | 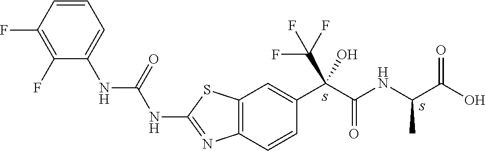 |
| 315 | 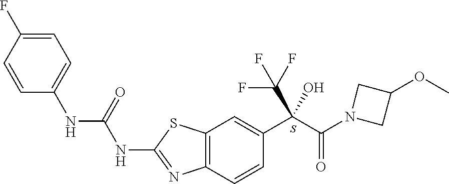 |
| 316 | 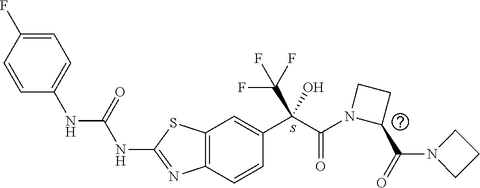 |
| 317 | 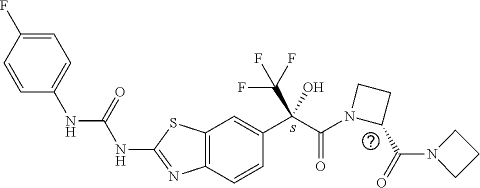 |
| 318 | 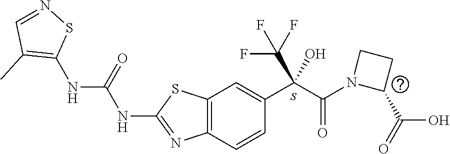 |
| 319 | 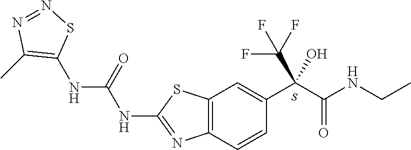 |
| 320 | 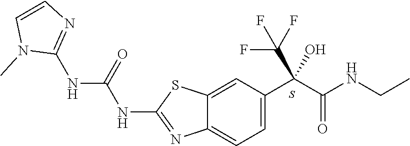 |
| 321 | 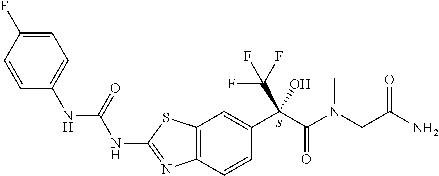 |
| 322 | 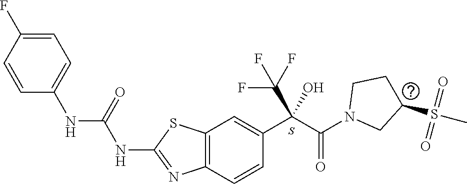 |
| 323 | 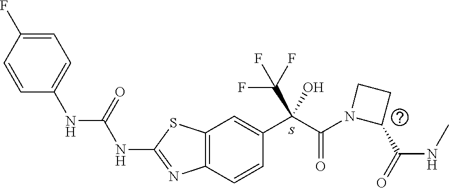 |
| 324 | 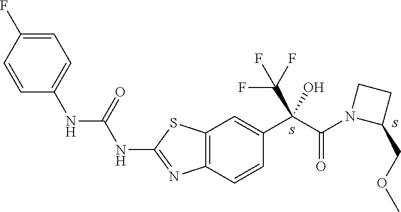 |
| 325 | 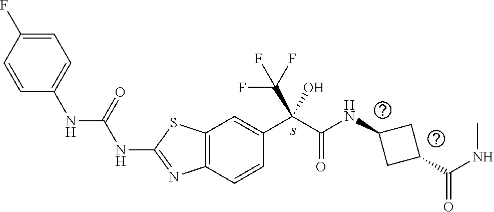 |
| 326 | 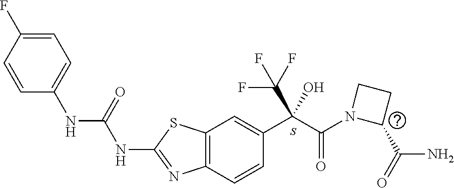 |
| 327 | 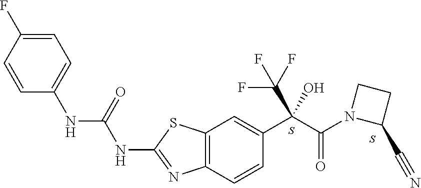 |
| 328 | 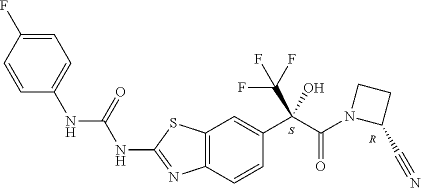 |
| 329 | 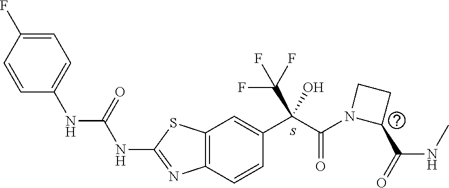 |
| 330 | 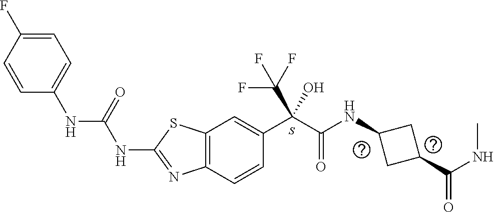 |
| 331 | 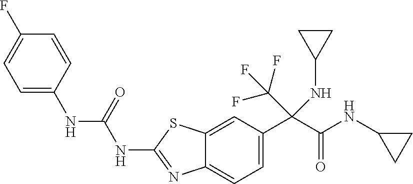 [SMILES: O=C(Nc1ccc(F)cc1)Nc1nc2ccc(C(NC3CC3)(C(=O)NC3CC3)C(F)(F)F)cc2s1] |
| 332 | 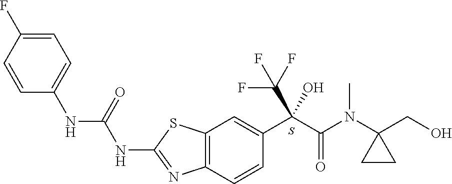 |
| 333 | 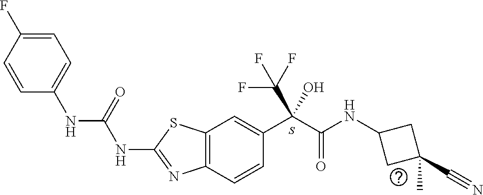 |
| 334 | 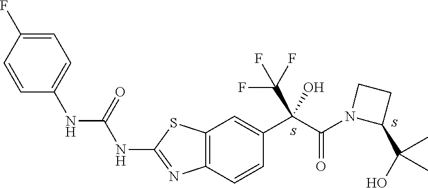 |
| 335 | 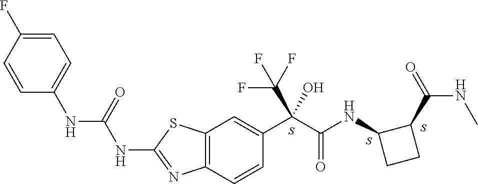 |
| 336 | 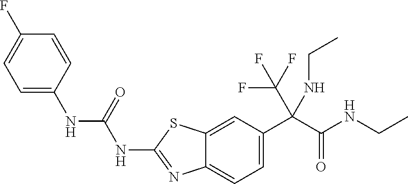 [SMILES: CCNC(=O)C(NCC)(c1ccc2nc(NC(=O)Nc3ccc(F)cc3)sc2c1)C(F)(F)F] |
| 337 | 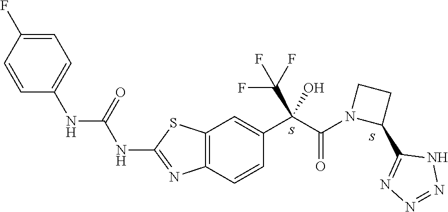 |
| 338 | 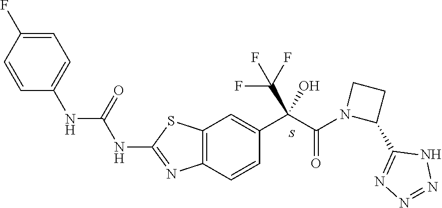 |
| 339 | 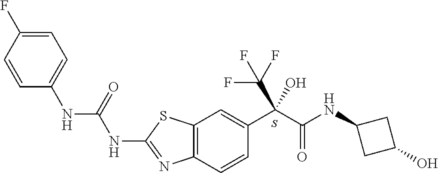 |
| 340 | 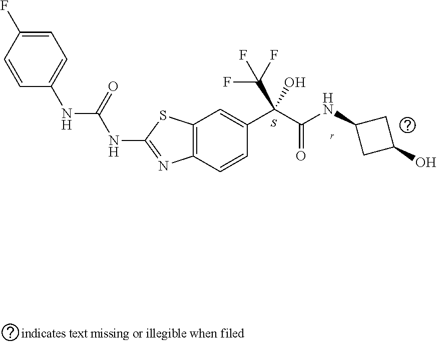 |
| 341 | 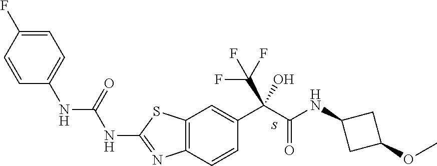 |
| 342 | 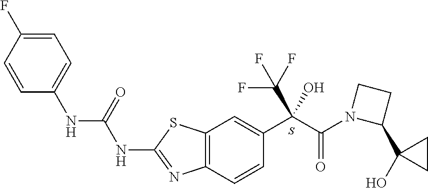 |
| 343 | 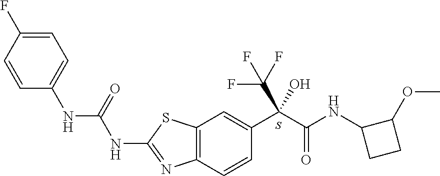 |
| 344 | 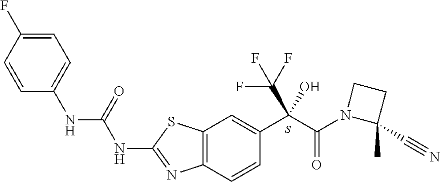 |
| 345 | 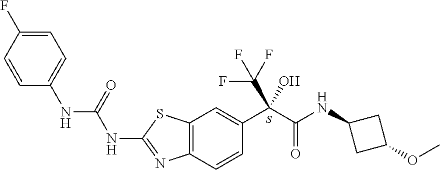 |
| 346 | 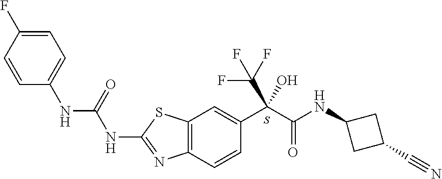 |
| 347 | 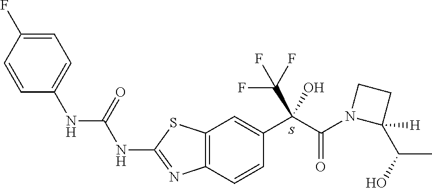 |
| 348 | 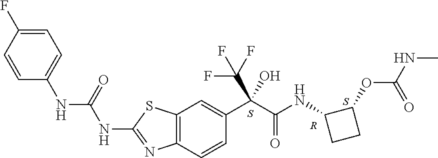 |
| 349 | 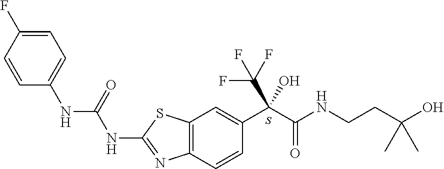 |
| 350 | 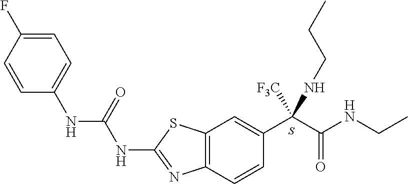 |
| 351 | 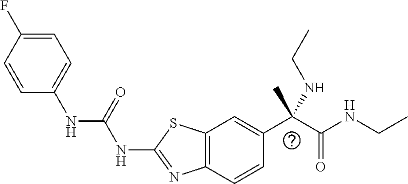 [SMILES: CCNC(=O)[C@@](C)(NCC)c1ccc2nc(NC(=O)Nc3ccc(F)cc3)sc2c1] |
| 352 | 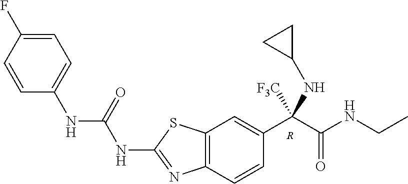 |
| 353 | 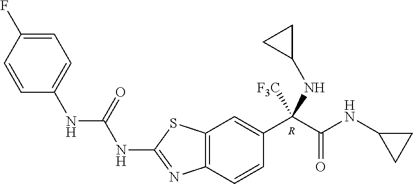 |
| 354 | 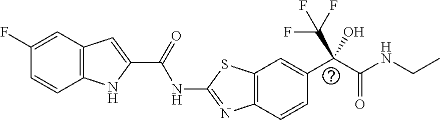 [SMILES: CCNC(=O)[C@@](O)(c1ccc2nc(NC(=O)c3cc4cc(F)ccc4[nH]3)sc2c1)C(F)(F)F] |
| 355 | 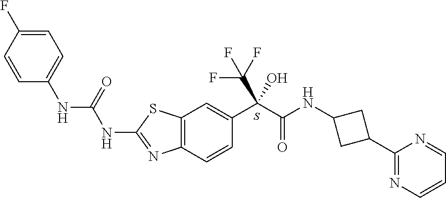 |
| 356 | 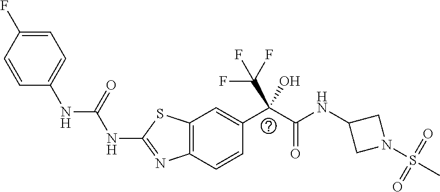 [SMILES: CS(=O)(=O)N1CC(NC(=O)[C@@](O)(c2ccc3nc(NC(=O)Nc4ccc(F)cc4)sc3c2)C(F)(F)F)C1] |
| 357 | 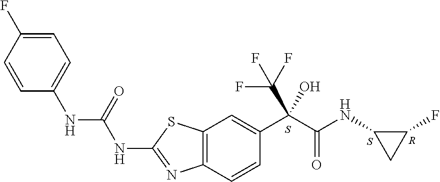 |
| 358 | 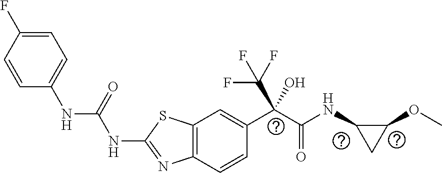 [SMILES: CO[C@H]1C[C@H]1NC(=O)[C@@](O)(c1ccc2nc(NC(=O)Nc3ccc(F)cc3)sc2c1)C(F)(F)F] |
| 359 | 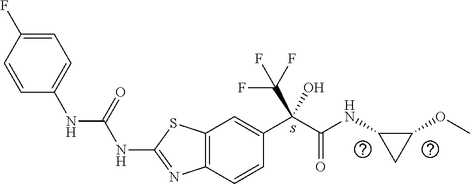 |
| 360 | 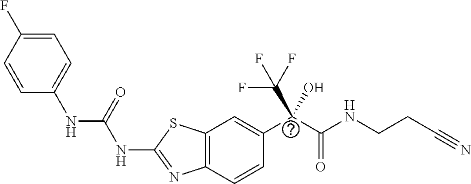 [SMILES: N#CCCNC(=O)[C@@](O)(c1ccc2nc(NC(=O)Nc3ccc(F)cc3)sc2c1)C(F)(F)F] |
| 361 | 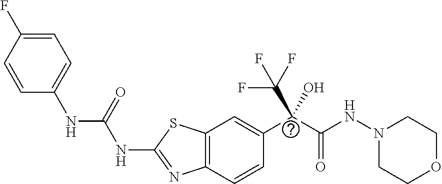 [SMILES: O=C(Nc1ccc(F)cc1)Nc1nc2ccc([C@](O)(C(=O)NN3CCOCC3)C(F)(F)F)cc2s1] |
| 362 | 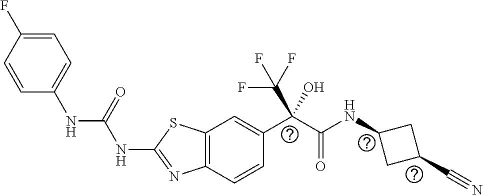 [SMILES: N#C[C@H]1C[C@@H](NC(=O)[C@@](O)(c2ccc3nc(NC(=O)Nc4ccc(F)cc4)sc3c2)C(F)(F)F)C1] |
| 363 | 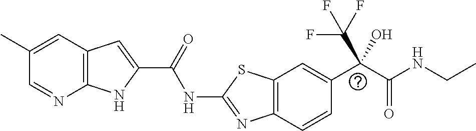 [SMILES: CCNC(=O)[C@@](O)(c1ccc2nc(NC(=O)c3cc4cc(C)cnc4[nH]3)sc2c1)C(F)(F)F] |
| 364 | 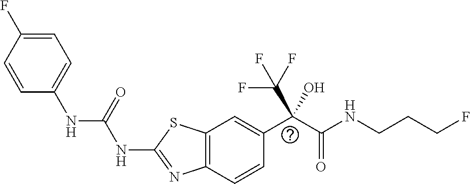 [SMILES: O=C(Nc1ccc(F)cc1)Nc1nc2ccc([C@](O)(C(=O)NCCCF)C(F)(F)F)cc2s1] |
| 365 | 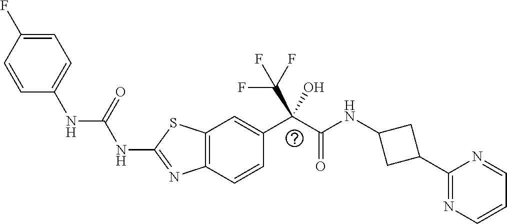 [SMILES: O=C(Nc1ccc(F)cc1)Nc1nc2ccc([C@](O)(C(=O)NC3CC(c4ncccn4)C3)C(F)(F)F)cc2s1] |
| 366 | 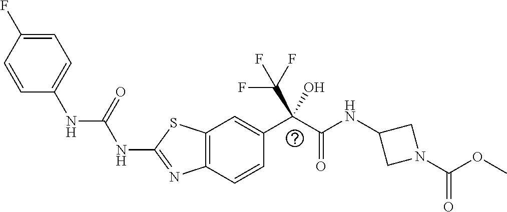 [SMILES: COC(=O)N1CC(NC(=O)[C@@](O)(c2ccc3nc(NC(=O)Nc4ccc(F)cc4)sc3c2)C(F)(F)F)C1] |
| 367 | 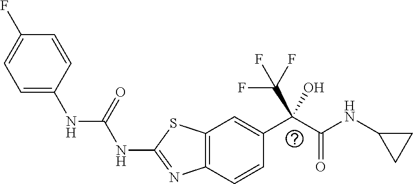 [SMILES: O=C(Nc1ccc(F)cc1)Nc1nc2ccc([C@](O)(C(=O)NC3CC3)C(F)(F)F)cc2s1] |
| 368 | 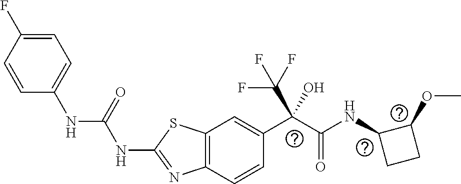 [SMILES: CO[C@H]1CC[C@H]1NC(=O)[C@@](O)(c1ccc2nc(NC(=O)Nc3ccc(F)cc3)sc2c1)C(F)(F)F] |
| 369 | 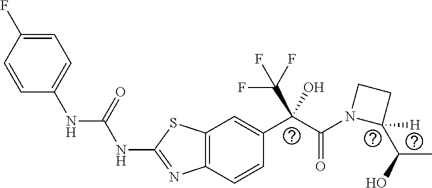 [SMILES: C[C@@H](O)[C@@H]1CCN1C(=O)[C@@](O)(c1ccc2nc(NC(=O)Nc3ccc(F)cc3)sc2c1)C(F)(F)F] |
| 370 | 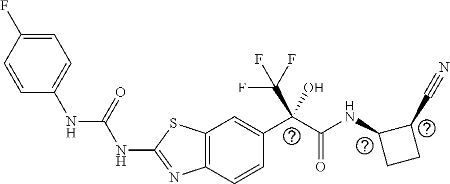 [SMILES: N#C[C@H]1CC[C@H]1NC(=O)[C@@](O)(c1ccc2nc(NC(=O)Nc3ccc(F)cc3)sc2c1)C(F)(F)F] |
| 371 | 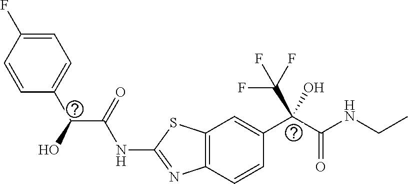 [SMILES: CCNC(=O)[C@@](O)(c1ccc2nc(NC(=O)[C@@H](O)c3ccc(F)cc3)sc2c1)C(F)(F)F] |
| 372 | 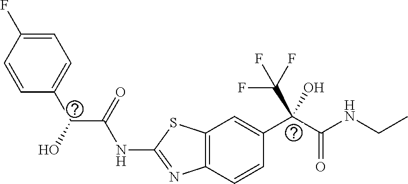 [SMILES: CCNC(=O)[C@@](O)(c1ccc2nc(NC(=O)[C@H](O)c3ccc(F)cc3)sc2c1)C(F)(F)F] |
| 373 | 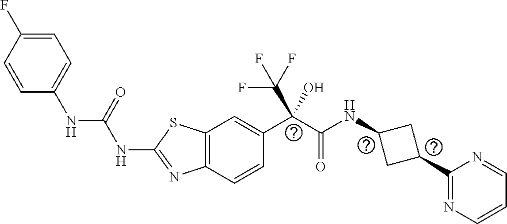 [SMILES: O=C(Nc1ccc(F)cc1)Nc1nc2ccc([C@](O)(C(=O)N[C@H]3C[C@@H](c4ncccn4)C3)C(F)(F)F)cc2s1] |
| 374 | 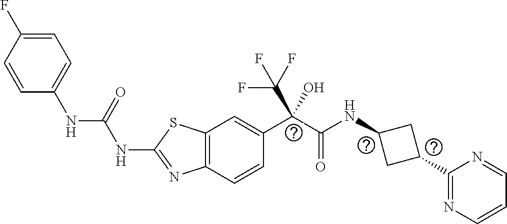 [SMILES: O=C(Nc1ccc(F)cc1)Nc1nc2ccc([C@](O)(C(=O)N[C@H]3C[C@H](c4ncccn4)C3)C(F)(F)F)cc2s1] |
| 375 | 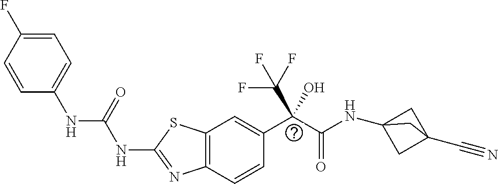 [SMILES: N#CC12CC(NC(=O)[C@@](O)(c3ccc4nc(NC(=O)Nc5ccc(F)cc5)sc4c3)C(F)(F)F)(C1)C2] |
| 376 | 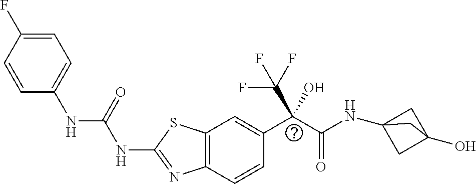 [SMILES: O=C(Nc1ccc(F)cc1)Nc1nc2ccc([C@](O)(C(=O)NC34CC(O)(C3)C4)C(F)(F)F)cc2s1] |
| 377 | 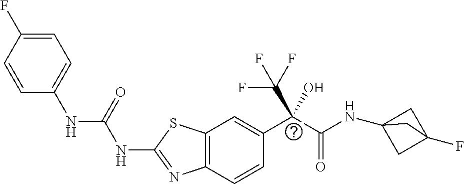 [SMILES: O=C(Nc1ccc(F)cc1)Nc1nc2ccc([C@](O)(C(=O)NC34CC(F)(C3)C4)C(F)(F)F)cc2s1] |
| 378 | 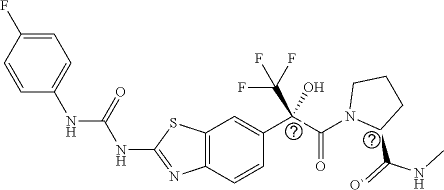 |
| 379 | 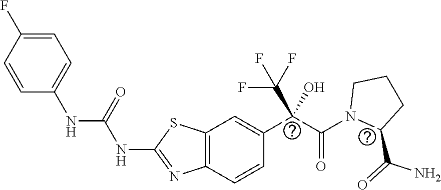 [SMILES: NC(=O)[C@@H]1CCCN1C(=O)[C@@](O)(c1ccc2nc(NC(=O)Nc3ccc(F)cc3)sc2c1)C(F)(F)F] |
| 380 | 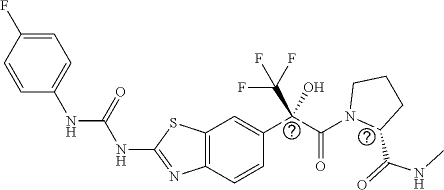 [SMILES: CNC(=O)[C@H]1CCCN1C(=O)[C@@](O)(c1ccc2nc(NC(=O)Nc3ccc(F)cc3)sc2c1)C(F)(F)F] |
| 381 | 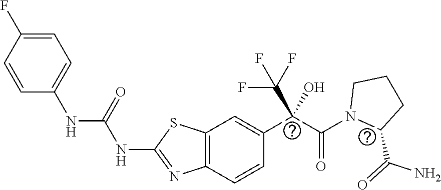 [SMILES: NC(=O)[C@H]1CCCN1C(=O)[C@@](O)(c1ccc2nc(NC(=O)Nc3ccc(F)cc3)sc2c1)C(F)(F)F] |
| 382 | 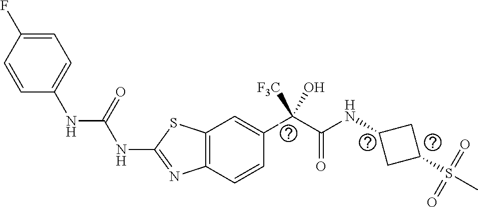 [SMILES: CS(=O)(=O)[C@H]1C[C@@H](NC(=O)[C@@](O)(c2ccc3nc(NC(=O)Nc4ccc(F)cc4)sc3c2)C(F)(F)F)C1] |
| 383 | 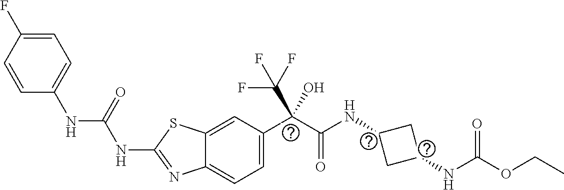 [SMILES: CCOC(=O)N[C@H]1C[C@@H](NC(=O)[C@@](O)(c2ccc3nc(NC(=O)Nc4ccc(F)cc4)sc3c2)C(F)(F)F)C1] |
| 384 | 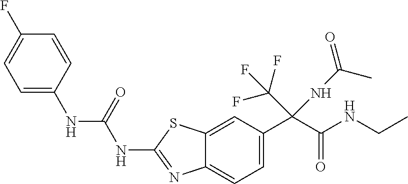 [SMILES: CCNC(=O)C(NC(C)=O)(c1ccc2nc(NC(=O)Nc3ccc(F)cc3)sc2c1)C(F)(F)F] |
| 385 | 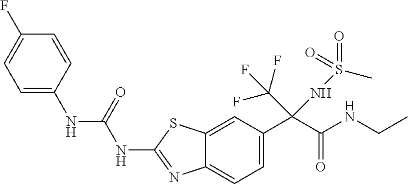 [SMILES: CCNC(=O)C(NS(C)(=O)=O)(c1ccc2nc(NC(=O)Nc3ccc(F)cc3)sc2c1)C(F)(F)F] |
| 386 | 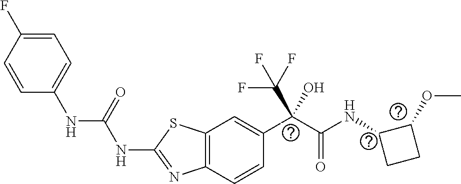 [SMILES: CO[C@@H]1CC[C@@H]1NC(=O)[C@@](O)(c1ccc2nc(NC(=O)Nc3ccc(F)cc3)sc2c1)C(F)(F)F] |
| 387 | 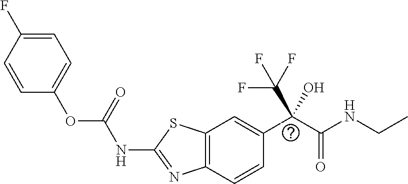 [SMILES: CCNC(=O)[C@@](O)(c1ccc2nc(NC(=O)Oc3ccc(F)cc3)sc2c1)C(F)(F)F] |
| 388 | 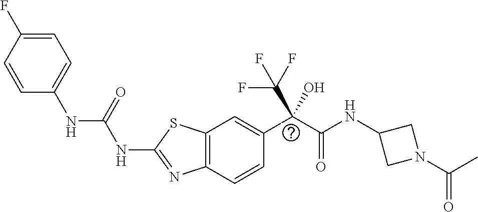 [SMILES: CC(=O)N1CC(NC(=O)[C@@](O)(c2ccc3nc(NC(=O)Nc4ccc(F)cc4)sc3c2)C(F)(F)F)C1] |
| 389 | 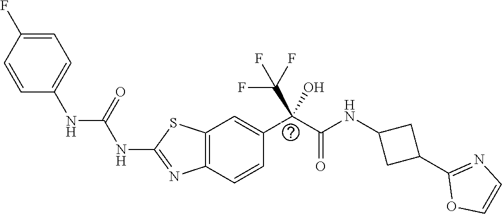 [SMILES: O=C(Nc1ccc(F)cc1)Nc1nc2ccc([C@](O)(C(=O)NC3CC(c4ncco4)C3)C(F)(F)F)cc2s1] |
| 390 | 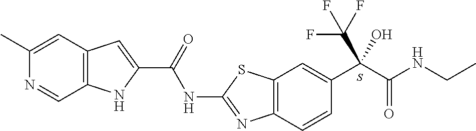 |
| 391 | 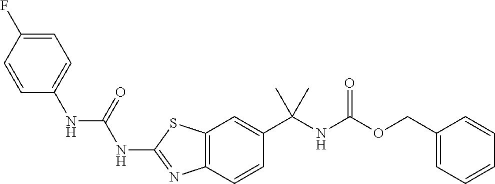 [SMILES: CC(C)(NC(=O)OCc1ccccc1)c1ccc2nc(NC(=O)Nc3ccc(F)cc3)sc2c1] |
| 392 | 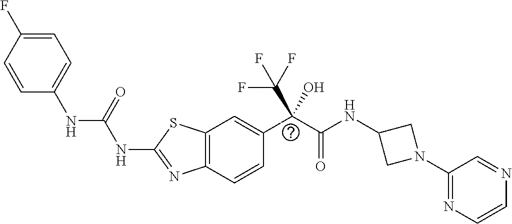 [SMILES: O=C(Nc1ccc(F)cc1)Nc1nc2ccc([C@](O)(C(=O)NC3CN(c4cnccn4)C3)C(F)(F)F)cc2s1] |
| 393 | 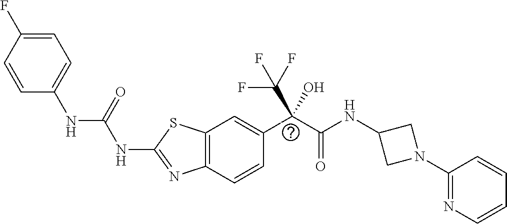 [SMILES: O=C(Nc1ccc(F)cc1)Nc1nc2ccc([C@](O)(C(=O)NC3CN(c4ccccn4)C3)C(F)(F)F)cc2s1] |
| 394 | 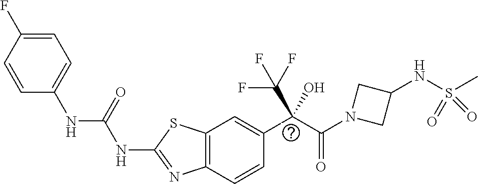 [SMILES: CS(=O)(=O)NC1CN(C(=O)[C@@](O)(c2ccc3nc(NC(=O)Nc4ccc(F)cc4)sc3c2)C(F)(F)F)C1] |
| 395 | 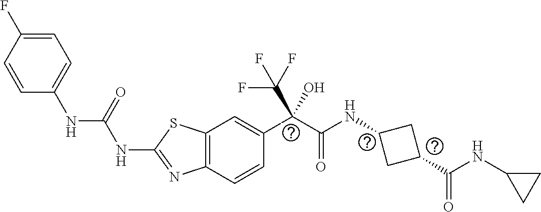 [SMILES: O=C(Nc1ccc(F)cc1)Nc1nc2ccc([C@](O)(C(=O)N[C@H]3C[C@@H](C(=O)NC4CC4)C3)C(F)(F)F)cc2s1] |
| 396 | 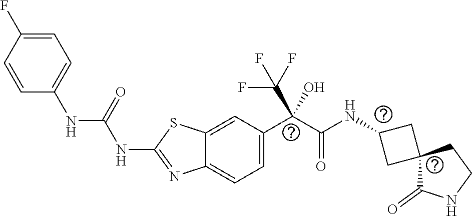 [SMILES: O=C(Nc1ccc(F)cc1)Nc1nc2ccc([C@](O)(C(=O)N[C@H]3C[C@]4(CCNC4=O)C3)C(F)(F)F)cc2s1] |
| 397 | 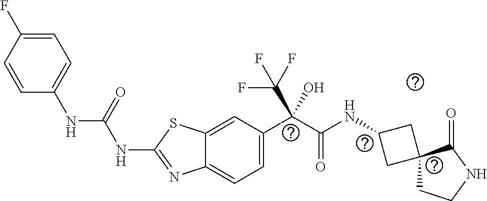 [SMILES: O=C(Nc1ccc(F)cc1)Nc1nc2ccc([C@](O)(C(=O)N[C@H]3C[C@@]4(CCNC4=O)C3)C(F)(F)F)cc2s1] |
| 398 | 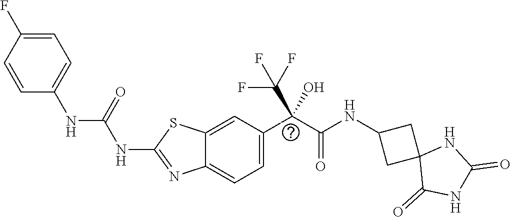 [SMILES: O=C(Nc1ccc(F)cc1)Nc1nc2ccc([C@](O)(C(=O)NC3CC4(C3)NC(=O)NC4=O)C(F)(F)F)cc2s1] |
| 399 | 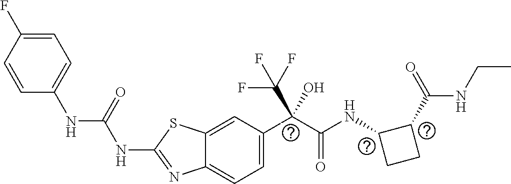 [SMILES: CCNC(=O)[C@@H]1CC[C@@H]1NC(=O)[C@@](O)(c1ccc2nc(NC(=O)Nc3ccc(F)cc3)sc2c1)C(F)(F)F] |
| 400 | 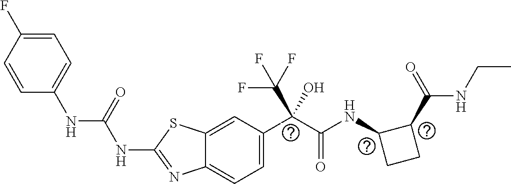 [SMILES: CCNC(=O)[C@H]1CC[C@H]1NC(=O)[C@@](O)(c1ccc2nc(NC(=O)Nc3ccc(F)cc3)sc2c1)C(F)(F)F] |
| 401 | 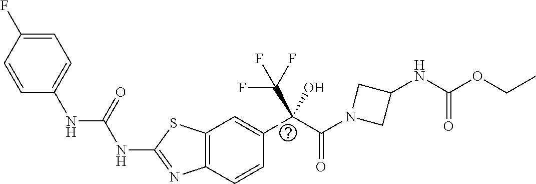 [SMILES: CCOC(=O)NC1CN(C(=O)[C@@](O)(c2ccc3nc(NC(=O)Nc4ccc(F)cc4)sc3c2)C(F)(F)F)C1] |
| 402 | 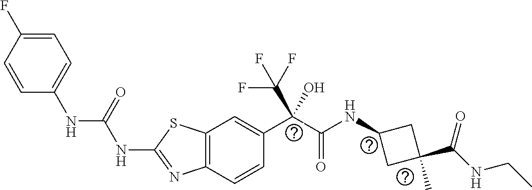 [SMILES: CCNC(=O)[C@]1(C)C[C@H](NC(=O)[C@@](O)(c2ccc3nc(NC(=O)Nc4ccc(F)cc4)sc3c2)C(F)(F)F)C1] |
| 403 | 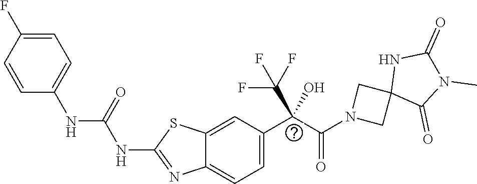 [SMILES: CN1C(=O)NC2(CN(C(=O)[C@@](O)(c3ccc4nc(NC(=O)Nc5ccc(F)cc5)sc4c3)C(F)(F)F)C2)C1=O] |
| 404 | 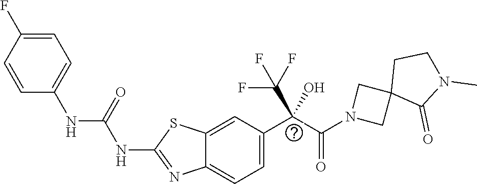 [SMILES: CN1CCC2(CN(C(=O)[C@@](O)(c3ccc4nc(NC(=O)Nc5ccc(F)cc5)sc4c3)C(F)(F)F)C2)C1=O] |
| 405 | 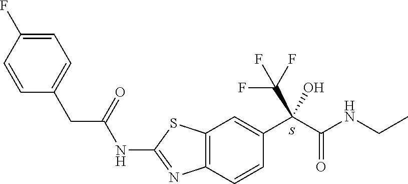 |
| 406 | 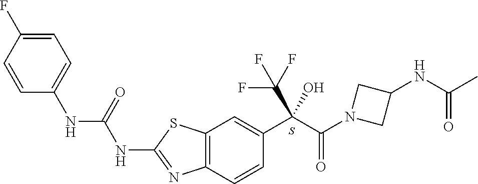 |
| 407 | 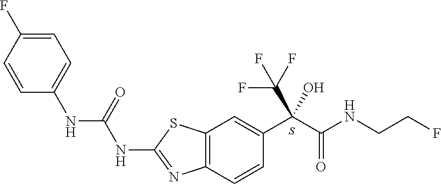 |
| 408 | 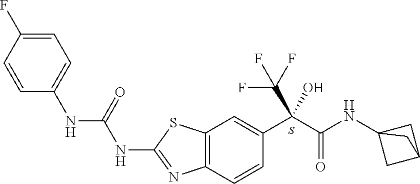 |
| 409 | 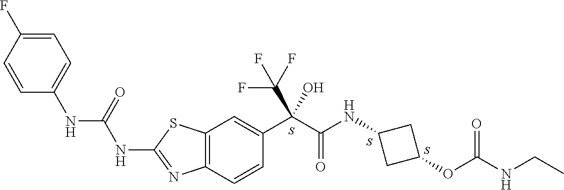 |
| 410 | 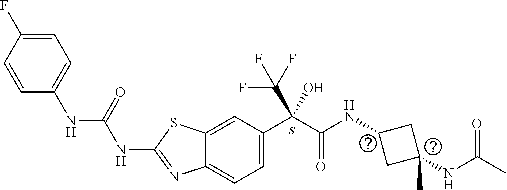 |
| 411 | 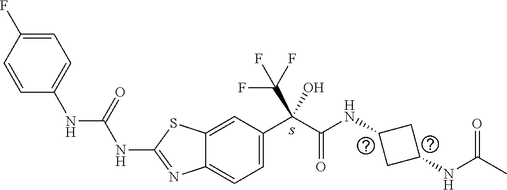 |
| 412 | 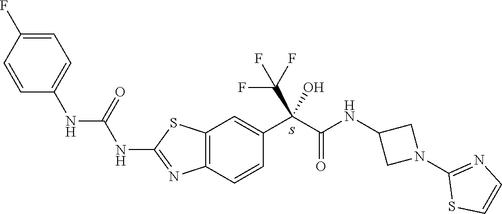 |
| 413 | 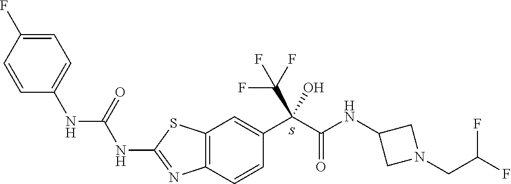 |
| 414 | 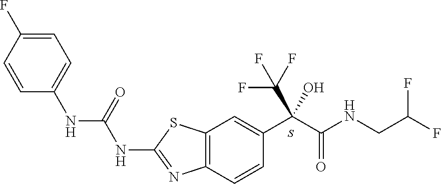 |
| 415 | 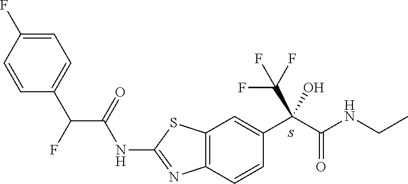 |
| 416 | 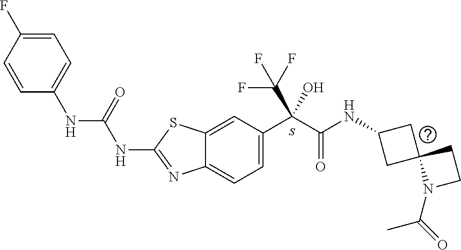 |
| 417 | 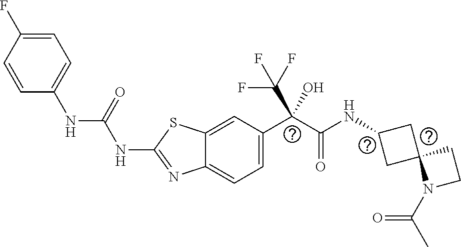 [SMILES: CC(=O)N1CC[C@]12C[C@@H](NC(=O)[C@@](O)(c1ccc3nc(NC(=O)Nc4ccc(F)cc4)sc3c1)C(F)(F)F)C2] |
| 418 | 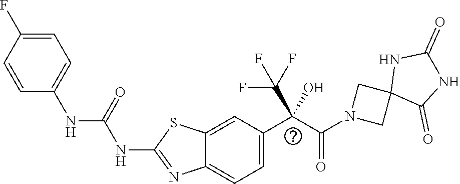 [SMILES: O=C(Nc1ccc(F)cc1)Nc1nc2ccc([C@](O)(C(=O)N3CC4(C3)NC(=O)NC4=O)C(F)(F)F)cc2s1] |
| 419 | 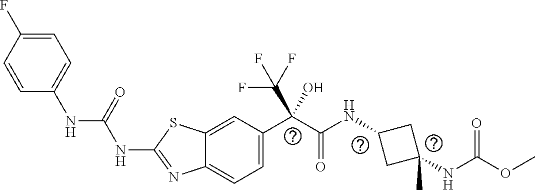 [SMILES: COC(=O)N[C@]1(C)C[C@H](NC(=O)[C@@](O)(c2ccc3nc(NC(=O)Nc4ccc(F)cc4)sc3c2)C(F)(F)F)C1] |
| 420 | 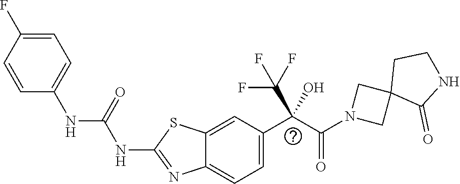 [SMILES: O=C(Nc1ccc(F)cc1)Nc1nc2ccc([C@](O)(C(=O)N3CC4(CCNC4=O)C3)C(F)(F)F)cc2s1] |
| 421 | 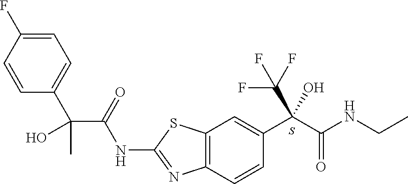 |
| 422 | 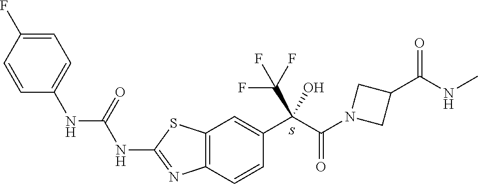 |
| 423 | 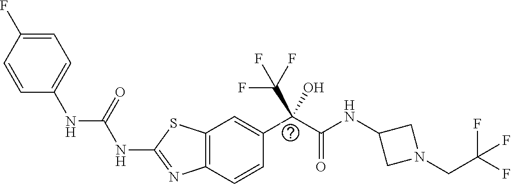 [SMILES: O=C(Nc1ccc(F)cc1)Nc1nc2ccc([C@](O)(C(=O)NC3CN(CC(F)(F)F)C3)C(F)(F)F)cc2s1] |
| 424 | 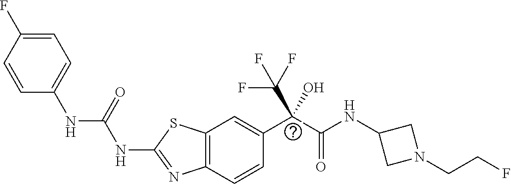 [SMILES: O=C(Nc1ccc(F)cc1)Nc1nc2ccc([C@](O)(C(=O)NC3CN(CCF)C3)C(F)(F)F)cc2s1] |
| 425 | 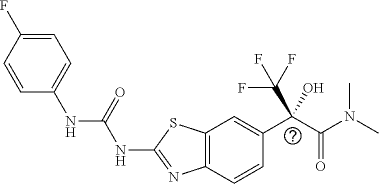 [SMILES: CN(C)C(=O)[C@@](O)(c1ccc2nc(NC(=O)Nc3ccc(F)cc3)sc2c1)C(F)(F)F] |
| 426 | 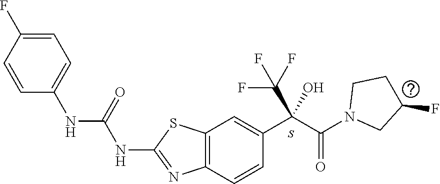 |
| 427 | 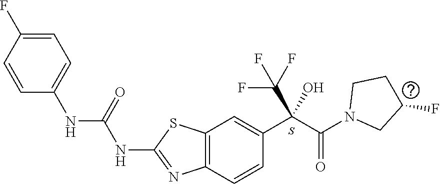 |
| 428 | 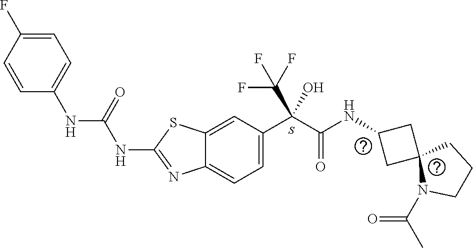 |
| 429 | 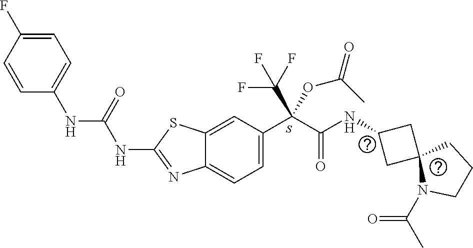 |
| 430 | 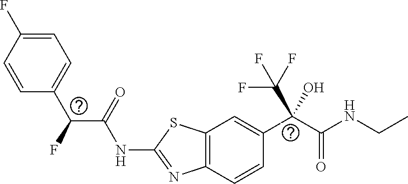 [SMILES: CCNC(=O)[C@@](O)(c1ccc2nc(NC(=O)[C@@H](F)c3ccc(F)cc3)sc2c1)C(F)(F)F] |
| 431 | 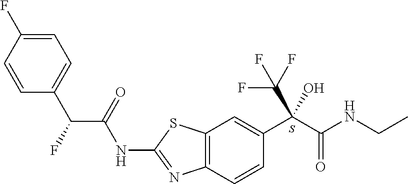 |
| 432 | 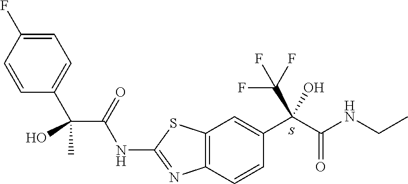 |
| 433 | 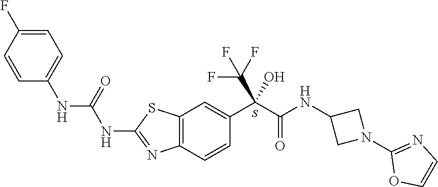 |
| 434 | 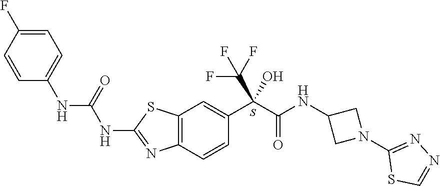 |
| 435 | 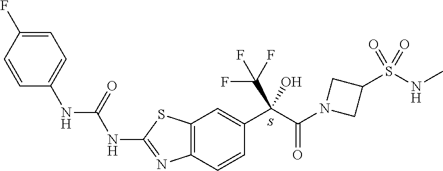 |
| 436 | 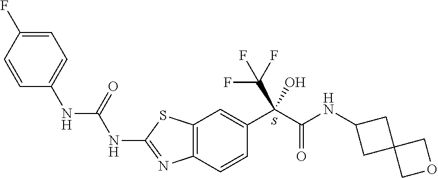 |
| 437 | 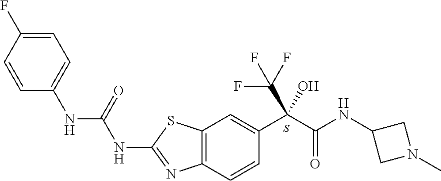 |
| 438 | 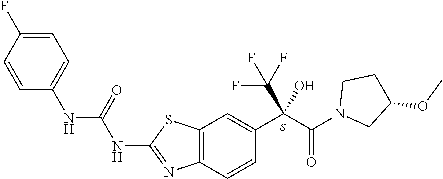 |
| 439 | 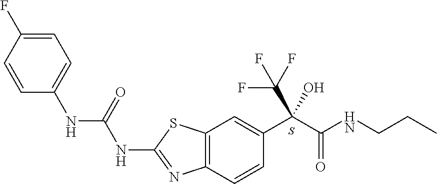 |
| 440 | 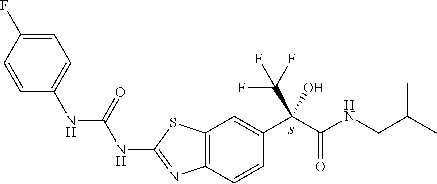 |
| 441 | 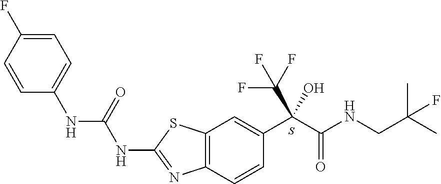 |
| 442 | 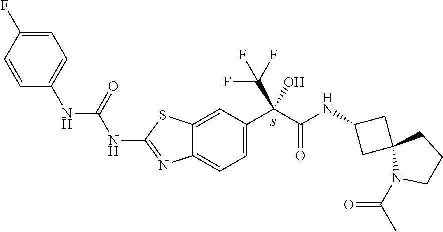 |
| 443 | 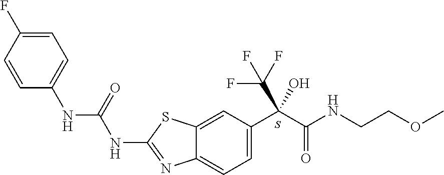 |
| 444 | 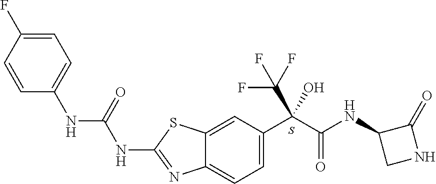 |
| 445 | 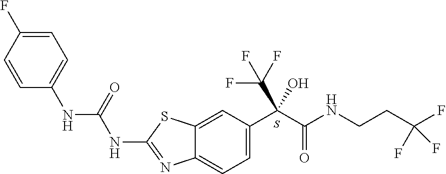 |
| 446 | 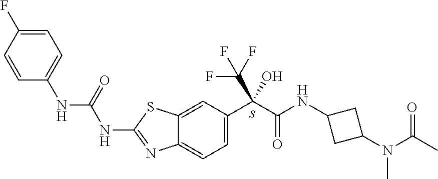 |
| 447 | 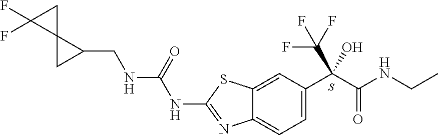 |
| 448 | 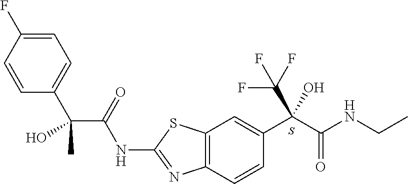 |
| 449 | 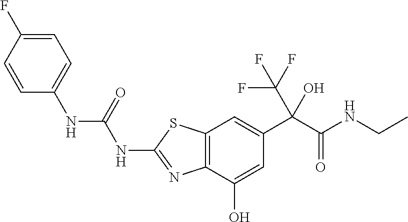 [SMILES: CCNC(=O)C(O)(c1cc(O)c2nc(NC(=O)Nc3ccc(F)cc3)sc2c1)C(F)(F)F] |
| 450 | 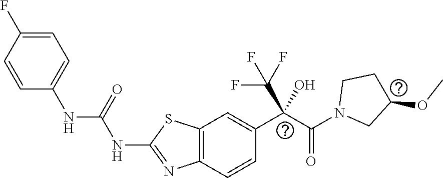 [SMILES: CO[C@@H]1CCN(C(=O)[C@@](O)(c2ccc3nc(NC(=O)Nc4ccc(F)cc4)sc3c2)C(F)(F)F)C1] |
| 451 | 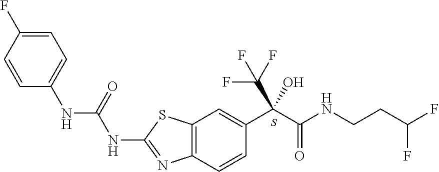 |
| 452 | 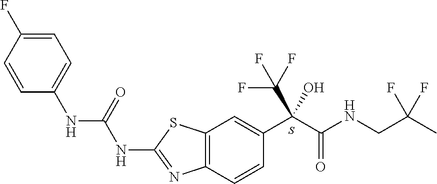 |
| 453 | 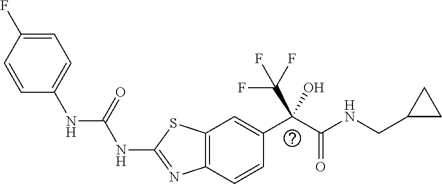 [SMILES: O=C(Nc1ccc(F)cc1)Nc1nc2ccc([C@](O)(C(=O)NCC3CC3)C(F)(F)F)cc2s1] |
| 454 | 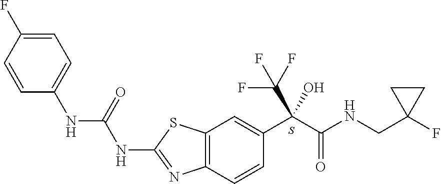 |
| 455 | 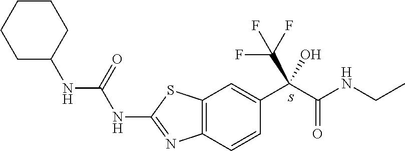 |
| 456 | 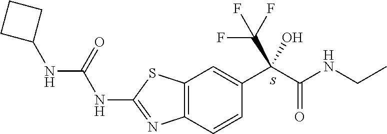 |
| 457 | 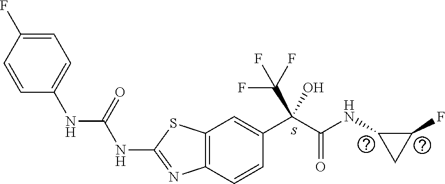 |
| 458 | 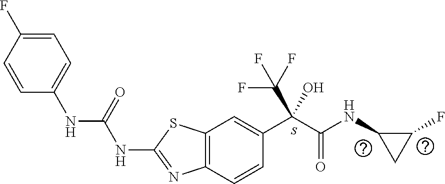 |
| 459 | 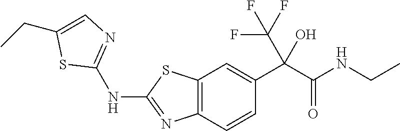 [SMILES: CCNC(=O)C(O)(c1ccc2nc(Nc3ncc(CC)s3)sc2c1)C(F)(F)F] |
| 460 | 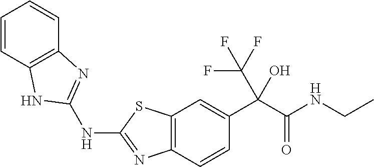 [SMILES: CCNC(=O)C(O)(c1ccc2nc(Nc3nc4ccccc4[nH]3)sc2c1)C(F)(F)F] |
| 461 | 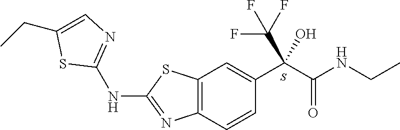 |
| 462 | 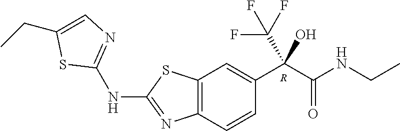 |
| 463 | 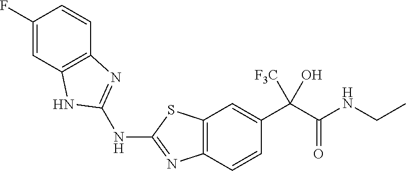 [SMILES: CCNC(=O)C(O)(c1ccc2nc(Nc3nc4ccc(F)cc4[nH]3)sc2c1)C(F)(F)F] |
| 464 | 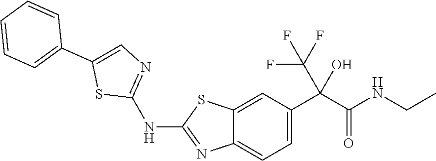 [SMILES: CCNC(=O)C(O)(c1ccc2nc(Nc3ncc(-c4ccccc4)s3)sc2c1)C(F)(F)F] |
| 465 | 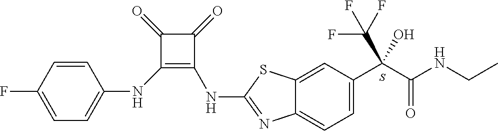 |
| 466 | 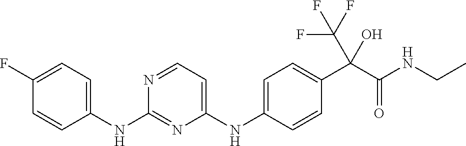 [SMILES: CCNC(=O)C(O)(c1ccc(Nc2ccnc(Nc3ccc(F)cc3)n2)cc1)C(F)(F)F] |
| 467 | 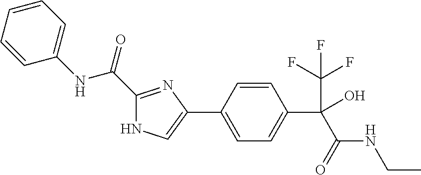 [SMILES: CCNC(=O)C(O)(c1ccc(-c2c[nH]c(C(=O)Nc3ccccc3)n2)cc1)C(F)(F)F] |
| 468 | 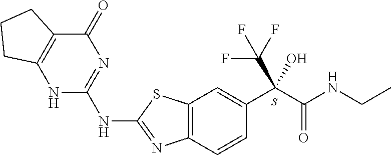 |
| 469 | 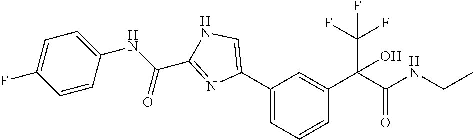 [SMILES: CCNC(=O)C(O)(c1cccc(-c2c[nH]c(C(=O)Nc3ccc(F)cc3)n2)c1)C(F)(F)F] |
| 470 | 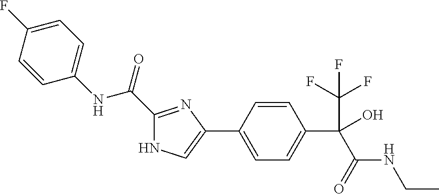 [SMILES: CCNC(=O)C(O)(c1ccc(-c2c[nH]c(C(=O)Nc3ccc(F)cc3)n2)cc1)C(F)(F)F] |
| 471 | 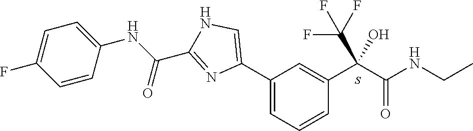 |
| 472 | 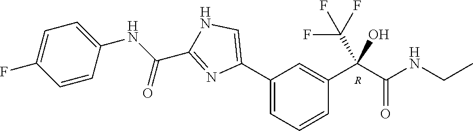 |
| 473 | 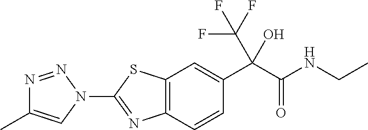 [SMILES: CCNC(=O)C(O)(c1ccc2nc(-n3cc(C)nn3)sc2c1)C(F)(F)F] |
| 474 | 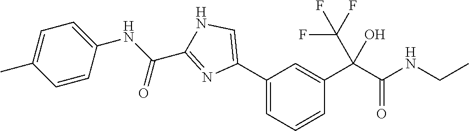 [SMILES: CCNC(=O)C(O)(c1cccc(-c2c[nH]c(C(=O)Nc3ccc(C)cc3)n2)c1)C(F)(F)F] |
| 475 | 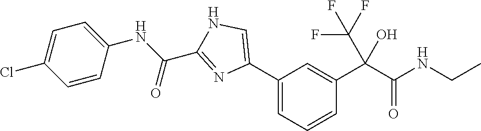 [SMILES: CCNC(=O)C(O)(c1cccc(-c2c[nH]c(C(=O)Nc3ccc(Cl)cc3)n2)c1)C(F)(F)F] |
| 476 | 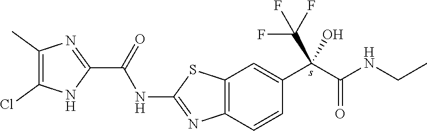 [SMILES: CCNC(=O)[C@@](O)(c1ccc2nc(NC(=O)c3nc(C)c(Cl)[nH]3)sc2c1)C(F)(F)F] |
| 477 | 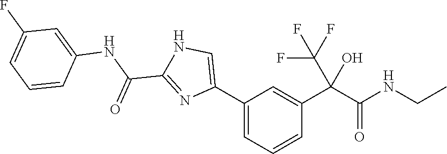 [SMILES: CCNC(=O)C(O)(c1cccc(-c2c[nH]c(C(=O)Nc3cccc(F)c3)n2)c1)C(F)(F)F] |
| 478 | 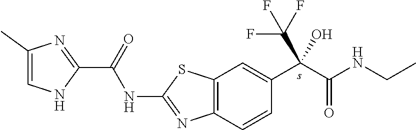 [SMILES: CCNC(=O)[C@@](O)(c1ccc2nc(NC(=O)c3nc(C)c[nH]3)sc2c1)C(F)(F)F] |
| 479 | 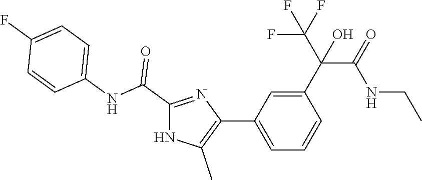 [SMILES: CCNC(=O)C(O)(c1cccc(-c2nc(C(=O)Nc3ccc(F)cc3)[nH]c2C)c1)C(F)(F)F] |
| 480 | 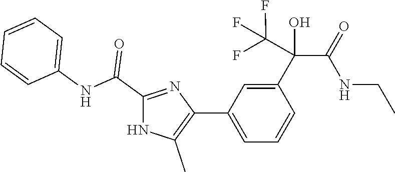 [SMILES: CCNC(=O)C(O)(c1cccc(-c2nc(C(=O)Nc3ccccc3)[nH]c2C)c1)C(F)(F)F] |
| 481 | 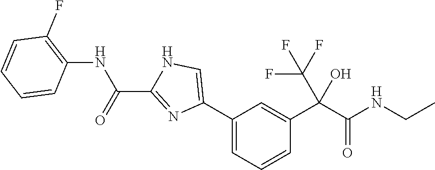 [SMILES: CCNC(=O)C(O)(c1cccc(-c2c[nH]c(C(=O)Nc3ccccc3F)n2)c1)C(F)(F)F] |
| 482 | 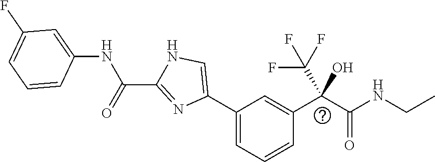 [SMILES: CCNC(=O)[C@](O)(c1cccc(-c2c[nH]c(C(=O)Nc3cccc(F)c3)n2)c1)C(F)(F)F] |
| 483 | 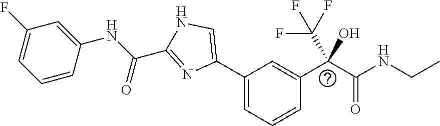 [SMILES: CCNC(=O)[C@](O)(c1cccc(-c2c[nH]c(C(=O)Nc3cccc(F)c3)n2)c1)C(F)(F)F] |
| 484 | 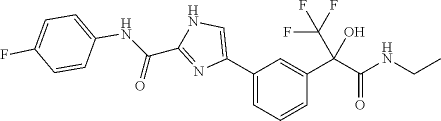 [SMILES: CCNC(=O)C(O)(c1cccc(-c2c[nH]c(C(=O)Nc3ccc(F)cc3)n2)c1)C(F)(F)F] |
| 485 | 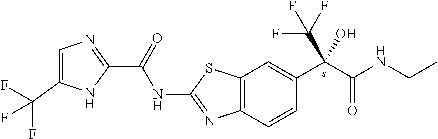 [SMILES: CCNC(=O)[C@@](O)(c1ccc2nc(NC(=O)c3ncc(C(F)(F)F)[nH]3)sc2c1)C(F)(F)F] |
| 486 | 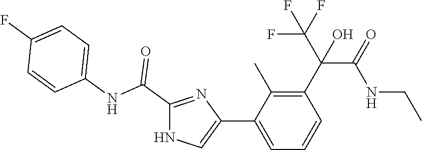 [SMILES: CCNC(=O)C(O)(c1cccc(-c2c[nH]c(C(=O)Nc3ccc(F)cc3)n2)c1C)C(F)(F)F] |
| 487 | 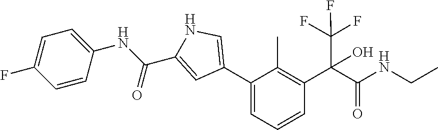 [SMILES: CCNC(=O)C(O)(c1cccc(-c2c[nH]c(C(=O)Nc3ccc(F)cc3)c2)c1C)C(F)(F)F] |
| 488 | 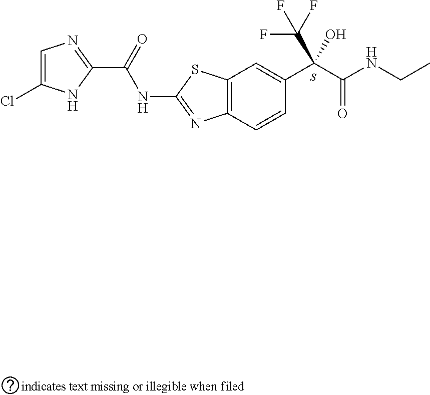 |
| 489 | 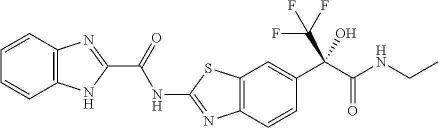 [SMILES: CCNC(=O)[C@@](O)(c1ccc2nc(NC(=O)c3nc4ccccc4[nH]3)sc2c1)C(F)(F)F] |
| 490 | 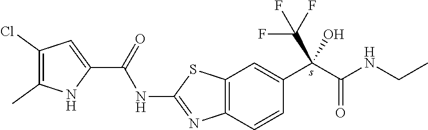 [SMILES: CCNC(=O)[C@@](O)(c1ccc2nc(NC(=O)c3cc(Cl)c(C)[nH]3)sc2c1)C(F)(F)F] |
| 491 | 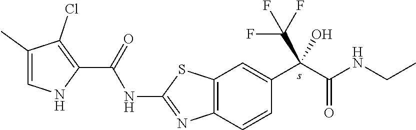 [SMILES: CCNC(=O)[C@@](O)(c1ccc2nc(NC(=O)c3[nH]cc(C)c3Cl)sc2c1)C(F)(F)F] |
| 492 | 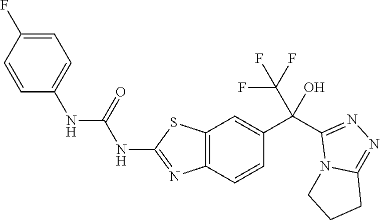 [SMILES: O=C(Nc1ccc(F)cc1)Nc1nc2ccc(C(O)(c3nnc4n3CCC4)C(F)(F)F)cc2s1] |
| 493 | 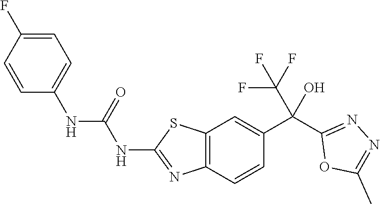 [SMILES: Cc1nnc(C(O)(c2ccc3nc(NC(=O)Nc4ccc(F)cc4)sc3c2)C(F)(F)F)o1] |
| 494 | 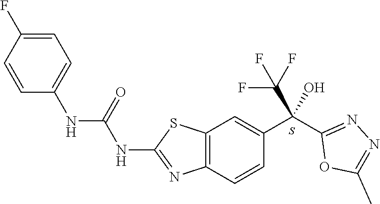 |
| 495 | 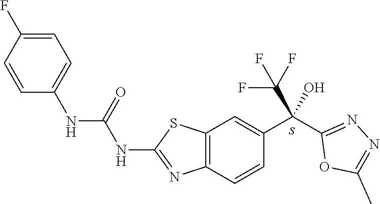 |
| 496 | 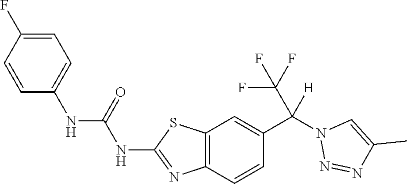 [SMILES: Cc1cn(C(c2ccc3nc(NC(=O)Nc4ccc(F)cc4)sc3c2)C(F)(F)F)nn1] |
| 497 | 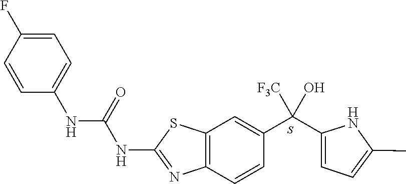 |
| 498 | 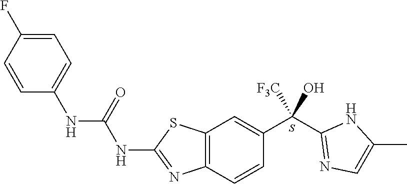 |
| 499 | 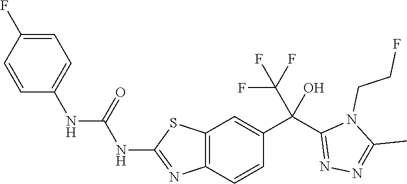 [SMILES: Cc1nnc(C(O)(c2ccc3nc(NC(=O)Nc4ccc(F)cc4)sc3c2)C(F)(F)F)n1CCF] |
| 500 | 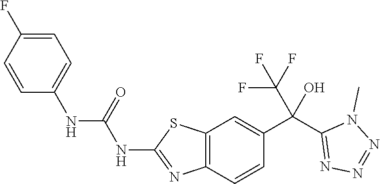 [SMILES: Cn1nnnc1C(O)(c1ccc2nc(NC(=O)Nc3ccc(F)cc3)sc2c1)C(F)(F)F] |
| 501 | 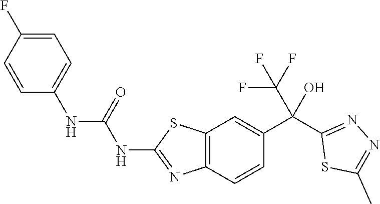 [SMILES: Cc1nnc(C(O)(c2ccc3nc(NC(=O)Nc4ccc(F)cc4)sc3c2)C(F)(F)F)s1] |
| 502 | 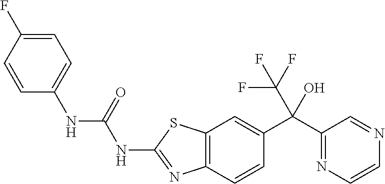 [SMILES: O=C(Nc1ccc(F)cc1)Nc1nc2ccc(C(O)(c3cnccn3)C(F)(F)F)cc2s1] |
| 503 | 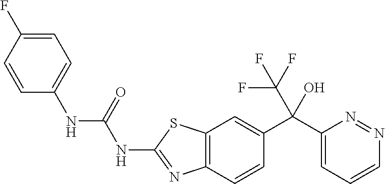 [SMILES: O=C(Nc1ccc(F)cc1)Nc1nc2ccc(C(O)(c3cccnn3)C(F)(F)F)cc2s1] |
| 504 | 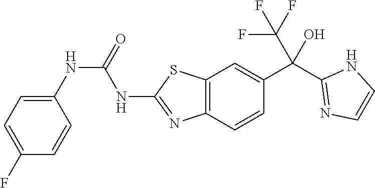 [SMILES: O=C(Nc1ccc(F)cc1)Nc1nc2ccc(C(O)(c3ncc[nH]3)C(F)(F)F)cc2s1] |
| 505 | 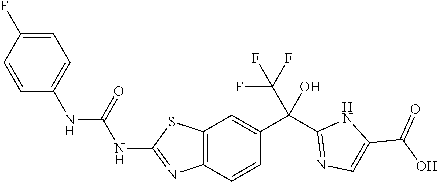 [SMILES: O=C(Nc1ccc(F)cc1)Nc1nc2ccc(C(O)(c3ncc(C(=O)O)[nH]3)C(F)(F)F)cc2s1] |

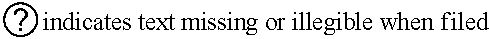

or a pharmaceutically acceptable salt thereof.

In certain embodiments, the compounds are atropisomers. Additionally, unless otherwise stated, structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention. Such compounds are useful, for example, as analytical tools, as probes in biological assays, or as therapeutic agents in accordance with the present invention. For example, in the case of variable R′, the (C₁-C₄)alkyl or the —O—(C₁-C₄)alkyl can be suitably deuterated (e.g., —CD₃, or —OCD₃, respectively).

Any compound of the invention can also be radiolabeled for the preparation of a radiopharmaceutical agent.

### Methods of Treatment

One aspect of the invention provides a method for treating or preventing osteoporosis, fracture, osteomalacia, arthritis, thrombocytopenia, hypoparathyroidism, hyperphosphatemia or tumoral calcinosis, comprising administering to a subject in need thereof an effective amount of a compound of Formula I, Ia, Ib, IIa, or IIb, or a pharmaceutically acceptable salt thereof.

Another aspect of this invention is a method for preventing or treating a condition mediated by PTH which comprises administering to a mammal in need thereof an effective amount of a compound of Formula I, Ia, Ib, IIa, or IIb, or a pharmaceutically acceptable salt thereof, either alone or in admixture with a pharmaceutically excipient. Another aspect of the invention includes a compound of Formula I, Ia, Ib, IIa, or IIb, or a pharmaceutically acceptable salt thereof, for use in the treatment and prevention of diseases and conditions characterized by loss of bone mineral density, mass, or strength, as well as in conditions wherein PTH would have a beneficial pharmacological effect. The invention includes administering a compound of Formula I, Ia, Ib, IIa, or IIb for use as a PTH mimetic. Another aspect of the invention includes use of a compound of Formula I, Ia, Ib, IIa, or IIb in the manufacture of a medicament for use in the treatment of osteopenia and osteoporosis in men and women for reduction in the risk of fractures, both vertebral and nonvertebral.

In certain embodiments, the compound is administered orally to the subject.

In certain embodiments, the compound is administered parenterally to the subject.

In certain embodiments, the disease is prevented. In other embodiments, the disease is treated.

### Pharmaceutical Compositions, Routes of Administration, and Dosing

In certain embodiments, the invention is directed to a pharmaceutical composition, comprising a compound of the invention, e.g., a compound of Formula I, Ia, Ib. IIa, or IIb; and a pharmaceutically acceptable carrier.

In certain embodiments, the invention is directed to a pharmaceutical composition, comprising a compound of any of the disclosed embodiments, and a pharmaceutically acceptable carrier.

In certain embodiments, the pharmaceutical composition comprises a plurality of compounds of the invention and a pharmaceutically acceptable carrier.

Pharmaceutical compositions of the invention can be prepared by combining one or more compounds of the invention with a pharmaceutically acceptable carrier and, optionally, one or more additional pharmaceutically active agents.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the invention being administered, the size of the subject, or the severity of the disease or condition. One of ordinary skill in the art can empirically determine the effective amount of a particular compound of the invention and/or other therapeutic agent without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

In certain embodiments, intravenous administration of a compound may typically be from 0.1 mg/kg/day to 20 mg/kg/day. In one embodiment, intravenous administration of a compound may typically be from 0.1 mg/kg/day to 2 mg/kg/day. In one embodiment, intravenous administration of a compound may typically be from 0.5 mg/kg/day to 5 mg/kg/day. In one embodiment, intravenous administration of a compound may typically be from 1 mg/kg/day to 20 mg/kg/day. In one embodiment, intravenous administration of a compound may typically be from 1 mg/kg/day to 10 mg/kg/day.

Generally, daily oral doses of a compound will be, for human subjects, from about 0.01 milligrams/kg per day to 1000 milligrams/kg per day. It is expected that oral doses in the range of 0.5 to 50 milligrams/kg, in one or more administrations per day, will yield therapeutic results. Dosage may be adjusted appropriately to achieve desired drug levels, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

For any compound described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the compound can be administered to a subject by any mode that delivers the compound to the desired surface. Administering a pharmaceutical composition may be accomplished by any means known to the skilled artisan. Routes of administration include but are not limited to intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound of the invention can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of acid hydrolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol moieties are suitable.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the compound of the invention (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compound of the invention (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, α-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex. STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the invention or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For topical administration, the compound may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

For administration by inhalation, compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

Also contemplated herein is pulmonary delivery of the compounds disclosed herein (or salts thereof). The compound is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13 (suppl. 5): 143-146 (1989) (endothelin-1); Hubbard et al., Annal Int Med 3:206-212 (1989) (□1-antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995 to Wong et al.

Contemplated for use in the practice of this invention are mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, North Carolina; and the Spinhaler powder inhaler, manufactured by Fisons Corp., Bedford, Mass.

All such devices require the use of formulations suitable for the dispensing of the compounds of the invention. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified compound of the invention may also be prepared in different formulations depending on the type of chemical modification or the type of device employed.

Formulations suitable for use with a nebulizer, either jet or ultrasonic, will typically comprise a compound of the invention (or derivative) dissolved in water at a concentration of about 0.1 to 25 mg of biologically active compound of the invention per mL of solution. The formulation may also include a buffer and a simple sugar (e.g., for inhibitor stabilization and regulation of osmotic pressure). The nebulizer formulation may also contain a surfactant, to reduce or prevent surface induced aggregation of the compound of the invention caused by atomization of the solution in forming the aerosol.

Formulations for use with a metered-dose inhaler device will generally comprise a finely divided powder containing the compound of the invention (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing a compound of the invention (or derivative) and may also include a bulking agent, such as lactose, sorbitol, sucrose, or mannitol in amounts which facilitate dispersal of the powder from the device, e.g., 50 to 90% by weight of the formulation. The compound of the invention (or derivative) should advantageously be prepared in particulate form with an average particle size of less than 10 micrometers (□m), most preferably 0.5 to 5 □m, for most effective delivery to the deep lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described above, a compound may also be formulated as a depot preparation. Such long acting formulations may be formulated with suitable polymeric or hydrophobic materials (for example, as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro) capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990).

The compound of the invention and optionally other therapeutics may be administered per se (neat) or in the form of a pharmaceutically acceptable salt or cocrystal. When used in medicine the salts or cocrystals should be pharmaceutically acceptable, but non-pharmaceutically acceptable salts or cocrystals may conveniently be used to prepare pharmaceutically acceptable salts or cocrystals thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the invention contain an effective amount of a compound as described herein and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agent(s), including specifically but not limited to a compound of the invention, may be provided in particles. Particles as used herein means nanoparticles or microparticles (or in some instances larger particles) which can consist in whole or in part of the compound of the invention or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the compound of the invention in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

The invention is further described in the following examples, which do not limit the scope of the invention described in the claims.

Abbreviations:

| | |
|---|---|
| ACN | Acetonitrile |
| AcOH | Acetic acid |
| Br₂ | Bromine |
| t-BuLi | tert-Butylithium |
| CDI | 1,1′-Carbonyldiimidazole |
| CDCl3 | Deuterated chloroform |
| Cs₂CO₃ | Cesium carbonate |
| CuSO₄ | Copper(II) sulfate |
| DCM | Dichloromethane |
| DIPEA | N,N-diisopropylethylamine |
| DMF | Dimethylformamide |
| DMSO | Dimethyl sulfoxide |
| EDC | 1-Ethyl-3-(3-dimethylaminopropyl)carbodiimide |
| EtOAc | Ethyl acetate |
| Fe | Iron |
| h | Hour |
| H₂ | Hydrogen |
| HATU | Hexafluorophosphate azabenzotriazole tetramethyl uronium |
| I₂ | Iodine |
| K₂CO₃ | Potassium carbonate |
| KSCN | Potassium thiocyanate |
| LiAlH₄ | Lithium aluminum hydride |
| LiOH | Lithium hydroxide |
| MeI | Methyl iodide |
| MeOH | Methanol |
| Min | Minute |
| MTBE | Methyl tertiary-butyl ether |
| N₂ | nitrogen |
| NaHCO₃ | Sodium bicarbonate |
| Na₂SO₄ | Sodium sulfate |
| NaH | Sodium hydride |
| NH₄Cl | Ammonium chloride |
| NH₄HCO₃ | Ammonium bicarbonate |
| NH₂OH•HCl | Hydroxylamine hydrochloride |
| Pd/C | Palladium on carbon |
| PE | Petroleum ether |
| PPh₃ | Triphenylphosphine |
| Rh₂(esp)₂ | Bis[rhodium(α,α,α′,α′-tetramethyl-1,3-benzenedipropionic |
| | acid)] |
| rt | Room temperature |
| TBAB | Tetrabutylammonium bromide |
| TEA | Triethylamine |
| TFA | Trifluoroacetic acid |
| THF | Tetrahydrofuran |
| TMSCN | Trimethylsilyl cyanide |

General schemes 1 and 2 were used to prepare certain compounds of the invention, which are described in further details in the experimental. In both schemes, R′ and R″ can be aryl, heteroaryl, alkyl, cycloalkyl, heterocycloalkyl etc. R″ can be H or R″; and R″ and R″ can form a ring.

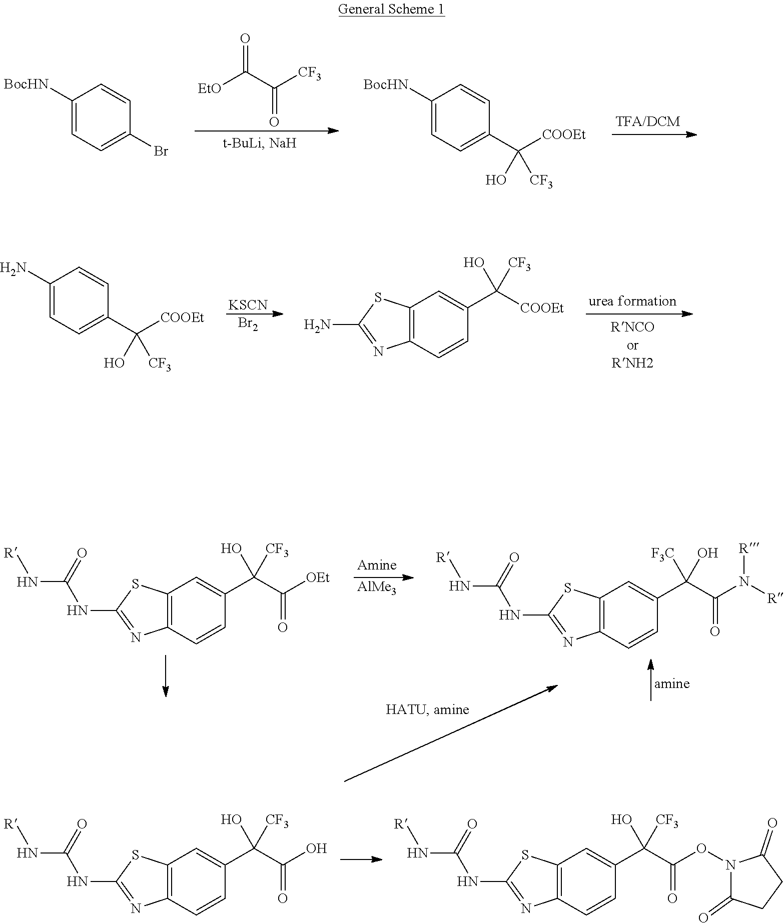

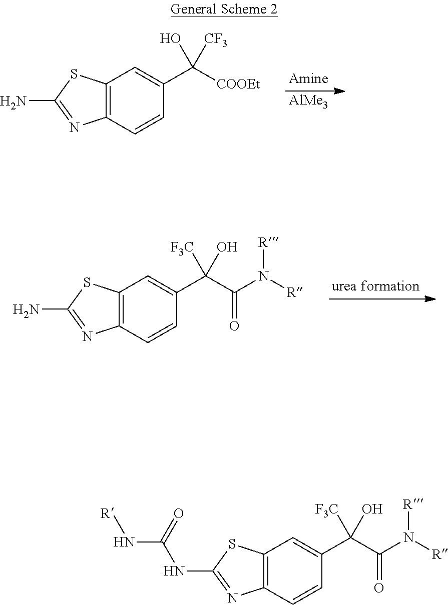

### EXPERIMENTAL PROCEDURES

### Example 2. Ethyl 2-(2-(3-cyclobutylureido)benzo[d]thiazol-6-yl)-3,3,3-trifluoro-2-hydroxypropanoate (2)

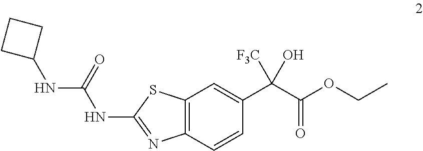 [SMILES: CCOC(=O)C(O)(c1ccc2nc(NC(=O)NC3CCC3)sc2c1)C(F)(F)F]

### Synthetic Scheme:

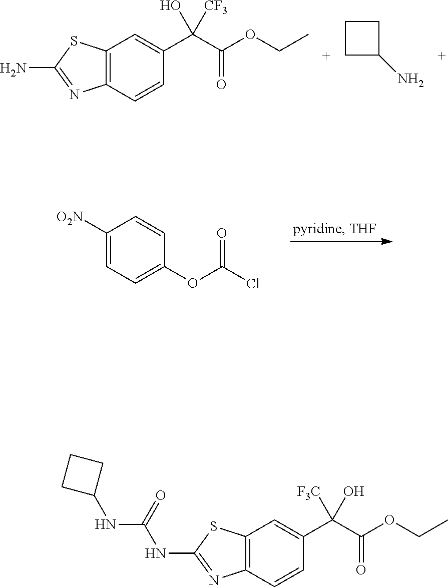

### Ethyl 2-(2-(3-cyclobutylurcido)benzo[d]thiazol-6-yl)-3,3,3-trifluoro-2-hydroxypropanoate

To a solution of ethyl 2-(2-amino-1,3-benzothiazol-6-yl)-3,3,3-trifluoro-2-hydroxy-propanoate (described in Example 6, 50 mg, 0.16 mmol) in THF (1.5 mL) was added pyridine (63.0 μL 0.78 mmol) and (4-nitrophenyl) carbonochloridate (62.9 mg, 0.31 mmol). After stirring at 25° C. for 30 min, cyclobutanamine (20.1 μL, 0.23 mmol) was added. After another 30 min, the reaction solvent was removed under reduced pressure. The resulting residue was purified by reverse phase HPLC (30-60% ACN/H₂O with 0.1% TFA as a modifier) to afford the title product (2) (10 mg, 15.4%, racemic) as a white solid. MS (ESI): mass calcd, for C₁₇H₁₈F₃N₃O₄S: 417.10, found: 418.00 [M+H]⁺. ¹H NMR (400 MHz, DMSO-d6) δ ppm 8.08 (s, 1H), 7.86 (s, 1H), 7.64 (d, J=8.8 Hz, 1H), 7.50 (d, J=8.8 Hz, 1H), 7.02 (d, J=6.8 Hz, 1H), 4.26-4.30 (m, 2H), 4.12-4.18 (m, 1H), 2.21-2.23 (m, 2H), 1.90-1.95 (m, 2H), 1.63-1.66 (m, 2H), 1.22 (t, J=7.2 Hz, 3H).

Examples 3, 4, 5 and were synthesized in similar procedures as described in Example 2.

### Examples 6 and 7. Ethyl (S)-3,3,3-trifluoro-2-hydroxy-2-(2-(3-phenylureido)benzo[d]thiazol-6-yl)propanoate (6) & Ethyl (R)-3,3,3-trifluoro-2-hydroxy-2-(2-(3-phenylureido)benzo[d]thiazol-6-yl)propanoate (7)

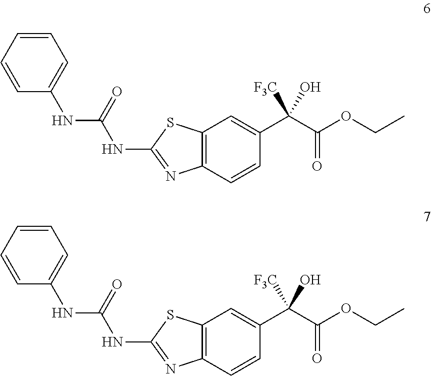 [SMILES: CCOC(=O)[C@@](O)(c1ccc2nc(NC(=O)Nc3ccccc3)sc2c1)C(F)(F)F ; CCOC(=O)[C@](O)(c1ccc2nc(NC(=O)Nc3ccccc3)sc2c1)C(F)(F)F]

### Synthetic Scheme:

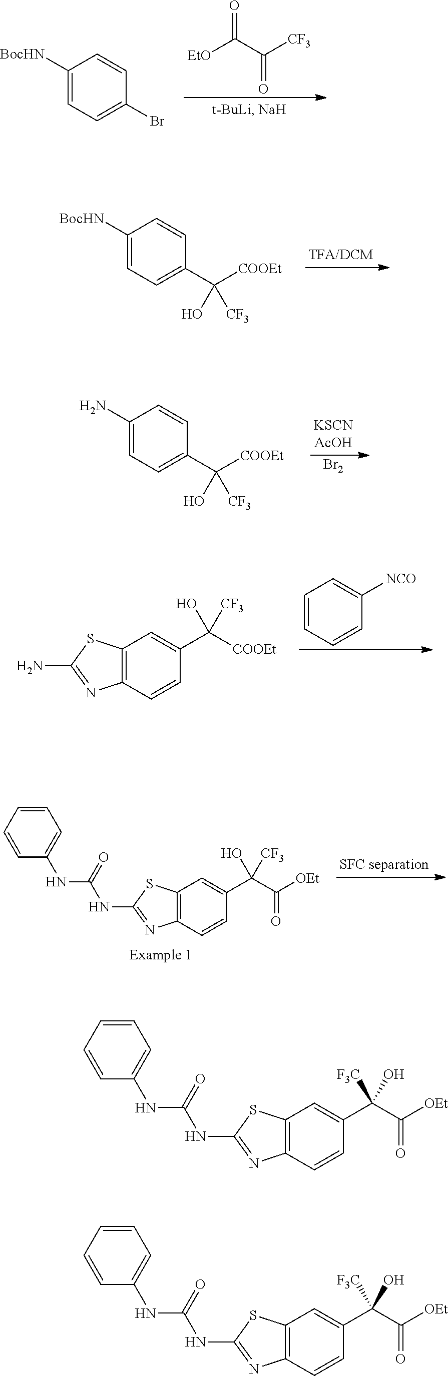

### Ethyl 2-(4-((tert-butoxycarbonyl)amino)phenyl)-3,3,3-trifluoro-2-hydroxypropanoate

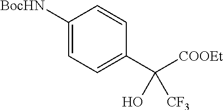 [SMILES: CCOC(=O)C(O)(c1ccc(NC(=O)OC(C)(C)C)cc1)C(F)(F)F]

To an ice cold solution of tert-butyl N-(4-bromophenyl)carbamate (1.35 g, 4.96 mmol) in THF (50 mL) was added NaH (198.4 mg, 4.96 mmol) under N₂. After stirring at 0° C. for 30 min, a solution of 1.3 M t-BuLi in heptane (5.8 mL, 7.54 mmol) was added dropwise at −70° C. The resulting solution was stirred at the same temperature for 30 min. Then ethyl 3,3,3-trifluoro-2-oxo-propanoate (1.27 g, 7.5 mmol) was added. After stirring at-70° C. for another 30 min, the reaction mixture was quenched with 50 mL of sat. NH₄Cl and extracted with ethyl acetate (50 mL×3). The combined organic layer was washed with brine (50 mL), dried over anhydrous Na₂SO₄, filtered, concentrated and purified by silica gel column chromatography (0-50% EtOAc/PE) to afford the title product (570 mg, 31.6%) as a yellow solid. MS (ESI): mass calcd, for C₁₆H₂₀F₃NO₅: 363.13, found: 362.3 [M−H]⁻.

### Ethyl 2-(4-aminophenyl)-3,3,3-trifluoro-2-hydroxypropanoate

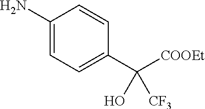 [SMILES: CCOC(=O)C(O)(c1ccc(N)cc1)C(F)(F)F]

A solution of ethyl 2-(4-((tert-butoxycarbonyl)amino)phenyl)-3,3,3-trifluoro-2-hydroxypropanoate (570 mg, 1.57 mmol) in TFA (3 mL) and DCM (3 mL) was stirred at 25° C. for 1 h. After completion, the reaction solvent was removed under reduced pressure. The resulting residue was diluted with DCM (10 mL) and washed with sat. NaHCO₃ (3 mL×2). The organic layer was dried over anhydrous Na₂SO₄, filtered and concentrated to afford the crude title product (400 mg, 96.8%) as a yellow oil, which was used in the next step without further purification. MS (ESI): mass calcd, for CH₁₁H₁₂F₃NO₃: 263.08, found: 264.1 [M+H]⁺.

### Ethyl 2-(2-aminobenzo[d]thiazol-6-yl)-3,3,3-trifluoro-2-hydroxypropanoate

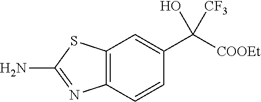 [SMILES: CCOC(=O)C(O)(c1ccc2nc(N)sc2c1)C(F)(F)F]

To a solution of ethyl 2-(4-aminophenyl)-3,3,3-trifluoro-2-hydroxy-propanoate (200 mg, 0.76 mmol) in AcOH (10 mL) was added KSCN (258.5 mg, 2.66 mmol). After stirring at 25° C. for 1 h, a solution of Br₂ (133.6 mg, 0.84 mmol) in AcOH (1 mL) was added. The reaction mixture was stirred at 25° C. for 12 h. After completion, the solution was filtered, and the filter cake was washed with EtOAc (10 mL×3). The filtrate was concentrated under reduced pressure. The resulting residue was treated with sat. NaHCO₃ (20 mL) to neutralize the remaining AcOH. The aqueous solution was extracted with EtOAc (20 mL×2). The combined organic layer was washed with brine (20 mL), dried over anhydrous Na₂SO₄, filtered, concentrated, and purified by silica gel column chromatography (1-50% EtOAc/PE) to afford the title product (200 mg, 82.2%) as a yellow solid. MS (ESI): mass calcd. for C₁₂H₁₁F₃N₂O₃S: 320.04, found: 319.2 [M−H]⁻.

### Ethyl 3,3,3-trifluoro-2-hydroxy-2-(2-(3-phenylureido)benzo[d]thiazol-6-yl)propanoate (Example 1)

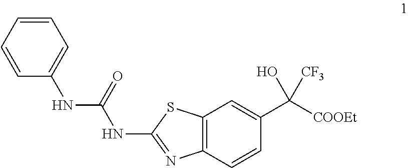 [SMILES: CCOC(=O)C(O)(c1ccc2nc(NC(=O)Nc3ccccc3)sc2c1)C(F)(F)F]

To a solution of ethyl 2-(2-aminobenzo[d]thiazol-6-yl)-3,3,3-trifluoro-2-hydroxypropanoate (800 mg, 2.50 mmol) in DMF (10 mL) was added phenyl isocyanate (405.7 μL, 3.75 mmol). After stirring at rt for 12 h, the reaction was quenched with H₂O (10 mL). The aqueous layer was extracted with EtOAc (10 mL×3). The combined organic layer was washed with brine (10 mL×3), dried over anhydrous Na₂SO₄, filtered and concentrated under reduced pressure to afford the crude title product (500 mg, racemic) as a yellow oil, which was used in the next step without further purification. Part of the crude was purified by silica gel column chromatography to obtain the pure title compound (1) for biological assay. MS (ESI): mass calcd, for C₁₀H₁₆F₃N₃O₄S: 439.08 found: 440.0 [M+H]⁺. ¹H NMR (400 MHz, DMSO-d6) δ ppm 10.85 (br s, 1H), 9.17 (s, 1H), 8.13 (s, 1H), 7.89 (s, 1H), 7.70 (d, J=5.6 Hz, 1H), 7.48-7.58 (m, 3H), 7.34 (t, J=8.0 Hz, 2H), 7.07 (t, J=7.2 Hz, 1H), 4.24-4.33 (m, 2H), 1.23 (t, J=7.2 Hz, 3H).

### Ethyl (S)-3,3,3-trifluoro-2-hydroxy-2-(2-(3-phenylureido)benzo[d]thiazol-6-yl)propanoate (6) & Ethyl (R)-3,3,3-trifluoro-2-hydroxy-2-(2-(3-phenylureido)benzo[d]thiazol-6-yl)propanoate (7)

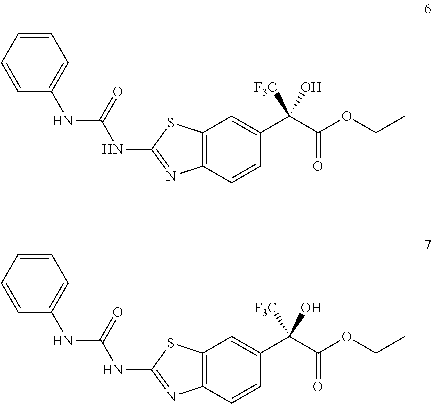 [SMILES: CCOC(=O)[C@@](O)(c1ccc2nc(NC(=O)Nc3ccccc3)sc2c1)C(F)(F)F ; CCOC(=O)[C@](O)(c1ccc2nc(NC(=O)Nc3ccccc3)sc2c1)C(F)(F)F]

Ethyl 3,3,3-trifluoro-2-hydroxy-2-(2-(3-phenylureido)benzo[d]thiazol-6-yl)propanoate (Example 1) was separated by chiral SFC separation (column: DAICEL CHIRALPAK AD (250 mm*30 mm, 10 μm); mobile phase: 48% EtOH with 0.1% NH₃H₂O) to afford the title compound (6) (23 mg, 0.046%) as a white solid. MS (ESI): mass calcd. for C₁₉H₁₆F₃N₃O₄S: 439.08, found: 440.0 [M+H]⁻. ¹H NMR (400 MHz, DMSO-d6) δ ppm 9.10-9.14 (m, 1H), 8.07 (s, 1H), 7.84 (s, 1H), 7.63 (d, J=18.4 Hz, 1H), 7.49-7.54 (m, 3H), 7.31 (t, J=7.6 Hz, 2H), 7.02 (t, J=6.8 Hz, 1H), 4.26-4.32 (m, 2H), 1.22 (t, J=6.8 Hz, 3H) and the title compound (7) (23 mg, 0.046%) as a white solid. MS (ESI): mass calcd. for C₁₉H₁₆F₃N₃O₄S: 439.08, found: 440.0 [M+H]⁻. ¹H NMR (400 MHz, DMSO-d6) δ ppm 9.17 (br s, 1H), 8.13 (s, 1H), 7.88 (s, 1H), 7.68 (br s, 1H), 7.51-7.55 (m, 3H), 7.33 (t, J=7.6 Hz, 2H), 7.02 (t, J=7.2 Hz, 1H), 4.26-4.32 (m, 2H), 1.22 (t, J=7.2 Hz, 3H).

Examples 18 and 21 were synthesized in similar procedures as described in Example 1.

### Example 8. Ethyl 2-(2-(3-(4-chlorophenyl)ureido)benzo[d]thiazol-6-yl)-3,3,3-trifluoro-2-hydroxypropanoate (8)

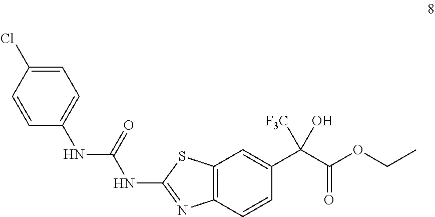 [SMILES: CCOC(=O)C(O)(c1ccc2nc(NC(=O)Nc3ccc(Cl)cc3)sc2c1)C(F)(F)F]

### Synthetic Scheme:

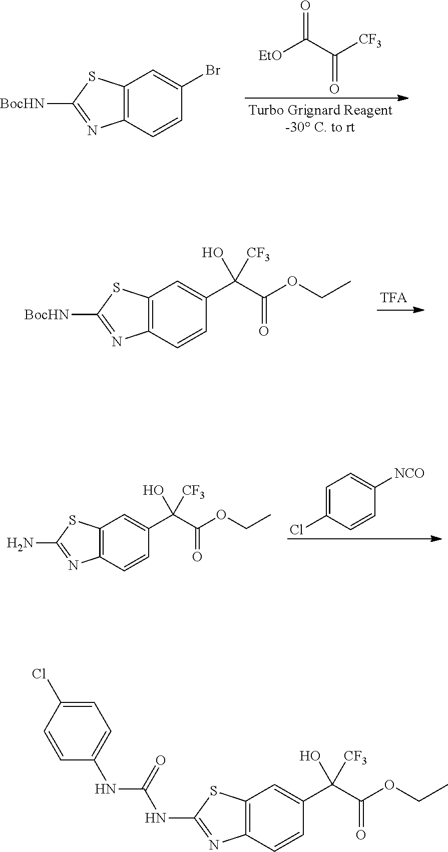

### Ethyl 2-(2-((tert-butoxycarbonyl)amino)benzo[d]thiazol-6-yl)-3,3,3-trifluoro-2-hydroxypropanoate

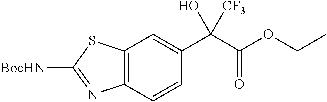 [SMILES: CCOC(=O)C(O)(c1ccc2nc(NC(=O)OC(C)(C)C)sc2c1)C(F)(F)F]

An oven dried vial was charged with 1.3 M ⁱPrMgCl-LiCl in THF (0.58 mL, 0.76 mmol). Tert-butyl (6-bromobenzo[d]thiazol-2-yl)carbamate (50 mg, 0.15 mmol) was added in one portion at rt. After 5 h, the reaction mixture was cooled to −10° C., and ethyl 3,3,3-trifluoro-2-oxopropanoate (0.058 mL, 0.46 mmol) was added. After stirring at 0° C. for 30 min, the reaction was quenched with sat. NH₄Cl (5 mL). The aqueous layer was extracted with EtOAc (10 mL×3). The combined organic layer was washed with brine (5 mL), dried over Na₂SO₄, filtered, concentrated and purified by silica gel column chromatography (gradient elution, 0-30% EtOAc in hexanes) to obtain the title product (20 mg, 31%) as a colorless oil. MS (ESI): mass calcd, for C₁₇H₁₉F₃N₂O₅S: 420.10, found: 421.00 [M+H]⁺.

### Ethyl 2-(2-aminobenzo[d]thiazol-6-yl)-3,3,3-trifluoro-2-hydroxypropanoate

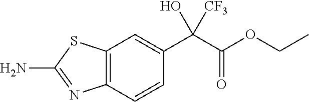 [SMILES: CCOC(=O)C(O)(c1ccc2nc(N)sc2c1)C(F)(F)F]

A solution of ethyl 2-(2-((tert-butoxycarbonyl)amino)benzo[d]thiazol-6-yl)-3,3,3-trifluoro-2-hydroxypropanoate (20 mg, 0.048 mmol) in TFA:DCM (1:1, 2 mL) was stirred at rt. After 10 min, the reaction solvent was removed under reduced pressure. The resulting residue was partitioned between DCM (10 mL) and sat. NaHCO₃ (5 mL). The aqueous layer was extracted with DCM (10 mL×2). The combined organic layer was washed with brine (5 mL), dried over Na₂SO₄, filtered and concentrated to afford the crude title product (10 mg) as a colorless oil, which was used in the next step without further purification. MS (ESI): mass calcd, for C₁₂H₁₁F₃N₂O₃S: 320.04, found: 320.90 [M+H]⁺.

### Ethyl 2-(2-(3-(4-chlorophenyl)ureido)benzo[d]thiazol-6-yl)-3,3,3-trifluoro-2-hydroxypropanoate

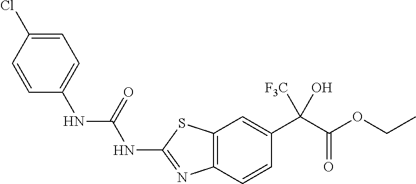 [SMILES: CCOC(=O)C(O)(c1ccc2nc(NC(=O)Nc3ccc(Cl)cc3)sc2c1)C(F)(F)F]

A solution of ethyl 2-(2-aminobenzo[d]thiazol-6-yl)-3,3,3-trifluoro-2-hydroxypropanoate (10 mg, 0.031 mmol) and 4-chlorophenyl isocyanate (6.23 mg, 0.041 mmol) in ACN (160 μL) was stirred at rt overnight. After completion, the reaction was quenched with sat. NH₄Cl (5 mL). The aqueous layer was extracted with DCM (10 mL×3). The combined organic layer was washed with brine (5 mL), dried over Na₂SO₄, filtered, concentrated and purified by silica gel column chromatography (gradient elution, 30-70% EtOAc in hexanes) to obtain the title product (8) (7.41 mg, 50%, racemic) as a colorless oil. MS (ESI): mass calcd, for C₁₉H₁₅ClF₃N₃O₄S: 473.04, found: 473.95 [M+H]⁺. ¹H NMR (400 MHz, CDCl₃): δ 8.21 (s, 1H), 7.88 (d, J=8.6 Hz, 1H), 7.75 (d, J=8.6 Hz, 1H), 7.48 (d, J=8.6 Hz, 2H), 7.31 (t, J=6.6 Hz, 2H), 4.37-4.53 (m, 2H), 1.39 (t, J=7.1 Hz, 3H).

Examples 9, 11, 16, 17 and 22 were synthesized in similar procedures as described in Example 8.

### Example 10. 3,3,3-Trifluoro-2-hydroxy-2-(2-(3-phenylureido)benzo[d]thiazol-6-yl)propanoic Acid (10)

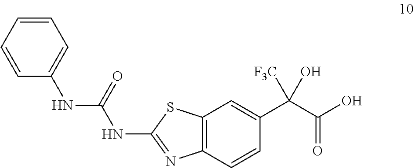 [SMILES: O=C(Nc1ccccc1)Nc1nc2ccc(C(O)(C(=O)O)C(F)(F)F)cc2s1]

### Synthetic Scheme:

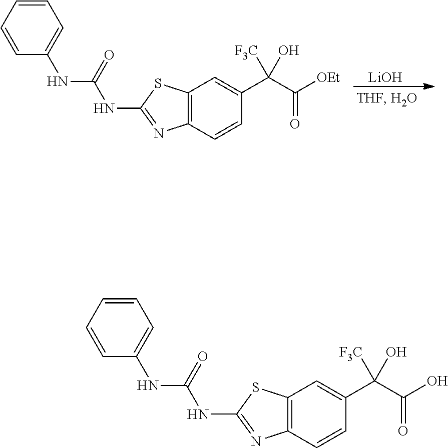

### 3,3,3-Trifluoro-2-hydroxy-2-(2-(3-phenylureido)benzo[d]thiazol-6-yl)propanoic Acid

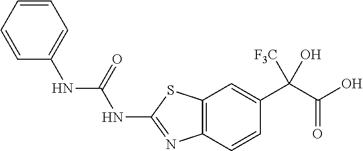 [SMILES: O=C(Nc1ccccc1)Nc1nc2ccc(C(O)(C(=O)O)C(F)(F)F)cc2s1]

A mixture of ethyl 3,3,3-trifluoro-2-hydroxy-2-[2-(phenylcarbamoylamino)-1,3-benzothiazol-6-yl]propanoate (Example 1, 50 mg, 0.11 mmol) in THF (1 mL) and H₂O (0.2 mL) was added LiOH·H₂O (7.16 mg, 0.17 mmol) and stirred at 25° C. for 12 h. After completion, the mixture was adjusted to pH=5-6 by 1 N HCl and extracted with EtOAc (5 mL×3). The combined organic layer was concentrated under reduced pressure. The resulting residue was purified by reverse phase HPLC (gradient elution, 15-45% ACN in H₂O, with 0.05% NH₄HCO₃ as a modifier) to afford the title product (10) (30 mg, 64.1%, racemic) as a white solid. MS (ESI): mass calcd, for C₁₇H₁₂F₃N₃O₄S: 411.05, found: 412.0 [M+H]⁺. ¹H NMR (400 MHz, DMSO-d6) δ ppm 10.93 (br s, 1H), 9.15 (s, 1H), 8.24-8.28 (s, 1H), 7.76 (d, J=8.8 Hz, 1H), 7.65 (d, J=8.0 Hz, 1H), 7.53 (d, J=8.0 Hz, 2H), 7.33 (t, J=8.0 Hz, 2H), 6.98-7.22 (m, 3H).

Examples 19 and 20 were synthesized through chiral SFC separation of Example 10.

### Example 13. N-Ethyl-3,3,3-trifluoro-2-hydroxy-2-(2-(3-phenylureido)benzo[d]thiazol-6-yl)propenamide (13)

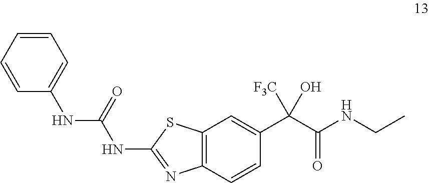 [SMILES: CCNC(=O)C(O)(c1ccc2nc(NC(=O)Nc3ccccc3)sc2c1)C(F)(F)F]

### Synthetic Scheme:

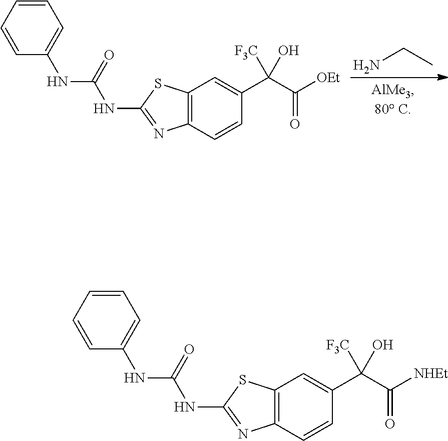

### N-Ethyl-3,3,3-trifluoro-2-hydroxy-2-(2-(3-phenylureido)benzo[d]thiazol-6-yl)propanamide

To a solution of ethanamine (69 μL, 1.05 mmol) in toluene (3 mL) was added trimethylaluminum (150.9 mg, 2.09 mmol) at 0° C. After stirring at 20° C. for 30 min, ethyl 3,3,3-trifluoro-2-hydroxy-2-[2-(phenylcarbamoylamino)-1,3-benzothiazol-6-yl]propanoate (Example 1, 0.23 g, 0.52 mmol) was added. The mixture was heated at 80° C. for 2 h under N₂. After completion, the reaction was quenched with ice water (5 mL). The aqueous phase was extracted with ethyl acetate (5 mL×3). The combined organic layer was washed with brine (5 mL), dried with anhydrous Na₂SO₄, filtered and concentrated to give the title product (13) (90 mg, racemic) as a yellow solid. A small portion was further purified for biological assay. MS (ESI): mass calcd, for C₁₉H₁₇F₃N₄O₃S: 438.10, found: 439.1 [M+H]⁺. ¹H NMR (400 MHz, DMSO-d6) δ ppm 10.41-11.17 (br s, 1H), 9.05-9.25 (br s, 1H), 8.08-8.28 (m, 2H), 7.76 (s, 1H), 7.65 (s, 2H), 7.52 (d, J=7.6 Hz, 2H), 7.33 (t, J=8.0 Hz, 2H), 7.05 (t, J=7.2 Hz, 1H), 3.09-3.16 (m, 2H), 0.99 (t, J=7.2 Hz, 3H).

Examples 42, 43, 44, 45, 46, and 54 were synthesized from Example 1 following the last step of Example 13.

Example 49 was synthesized in similar procedures as described in Example 13.

Examples 50 and 51 were synthesized from Example 49 by chiral SFC separation (DAICEL CHIRALPAK IG (250 mm*30 mm, 10 μm); 45% iPrOH with 0.1% NH₃H₂O).

Example 70, 71, and 75 was synthesized from their respective chiral ester precursors following the last step of Example 13.

### Example 14. 1-Phenyl-3-(6-(1,1,1-trifluoro-2,3-dihydroxypropan-2-yl)benzo[d]thiazol-2-yl)urea (14)

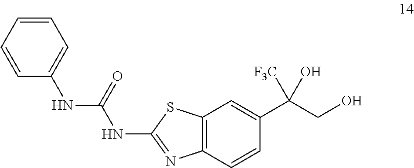 [SMILES: O=C(Nc1ccccc1)Nc1nc2ccc(C(O)(CO)C(F)(F)F)cc2s1]

### Synthetic Scheme:

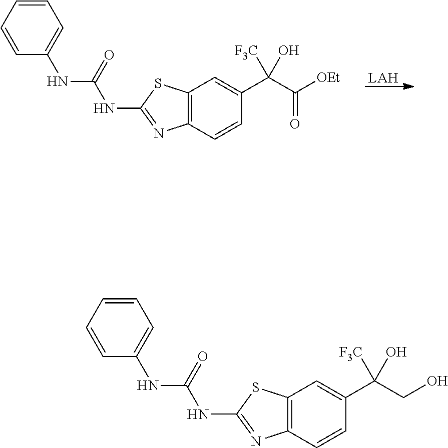 [SMILES: CCOC(=O)C(O)(c1ccc2nc(NC(=O)Nc3ccccc3)sc2c1)C(F)(F)F ; "LAH" ; O=C(Nc1ccccc1)Nc1nc2ccc(C(O)(CO)C(F)(F)F)cc2s1]

### 1-Phenyl-3-(6-(1,1,1-trifluoro-2,3-dihydroxypropan-2-yl)benzo[d]thiazol-2-yl)urea

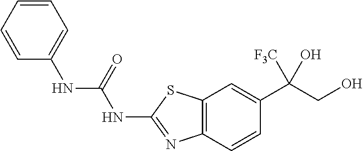 [SMILES: O=C(Nc1ccccc1)Nc1nc2ccc(C(O)(CO)C(F)(F)F)cc2s1]

To an ice cold solution of ethyl 3,3,3-trifluoro-2-hydroxy-2-[2-(phenylcarbamoylamino)-1,3-benzothiazol-6-yl]propanoate (Example 1, 50 mg, 0.11 mmol) in THF (1 mL) was added LiAlH₄ (8.64 mg, 0.23 mmol) under N₂. After stirring at rt for 12 h, the reaction was quenched by dropwise addition of H₂O (0.1 mL), 15% NaOH solution (0.1 mL) and H₂O (0.3 mL). After stirring for 2 min, the crude mixture was dried over anhydrous Na₂SO₄, filtered, concentrated and purified by reverse phase HPLC (gradient elution, 25-55% ACN in H₂O, with 0.1% TFA as a modifier) to afford the title product (14) (3 mg, 6.6%, racemic) as a white solid. MS (ESI): mass calcd, for C₁₇H₁₄F₃N₃O₃S: 397.07, found: 398.1 [M+H]⁺. ¹H NMR (400 MHz, DMSO-d₆) δ ppm 10.86 (br s, 1H), 9.20 (s, 1H). 8.12 (s, 1H), 7.60-7.64 (m, 2H), 7.52 (d, J=7.6 Hz, 2H), 7.32-7.34 (m, 2H), 7.05-7.07 (m, 1H), 6.51 (br s, 1H), 3.95 (s, 2H).

### Example 23 and 24. (R)—N-Ethyl-3,3,3-trifluoro-2-hydroxy-2-(2-(3-phenylureido)benzo[d]thiazol-6-yl)propenamide (23) & (S)—N-Ethyl-3,3,3-trifluoro-hydroxy-2-(2-(3-phenylureido)benzo[d]thiazol-6-yl)propenamide (24)

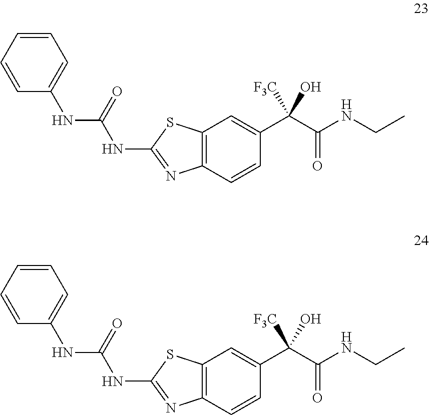 [SMILES: CCNC(=O)[C@](O)(c1ccc2nc(NC(=O)Nc3ccccc3)sc2c1)C(F)(F)F ; CCNC(=O)[C@@](O)(c1ccc2nc(NC(=O)Nc3ccccc3)sc2c1)C(F)(F)F]

N-ethyl-3,3,3-trifluoro-2-hydroxy-2-(2-(3-phenylureido)benzo[d]thiazol-6-yl)propenamide (Example 13) was separated by chiral SFC column: ChiralPak IH, 250*30 mm, 10 μm; mobile phase: 21% EtOH with 0.1% NH₃H₂O) to provide the title product (23), MS (ESI): mass calcd, for C₁₉H₁₇F₃N₄O₃S: 438.10, found: 439.00 [M+H]⁺. ¹H NMR (400 MHz, DMSO-d6) δ ppm 10.76 (s, 1H), 9.18 (br s, 1H), 8.13-8.26 (m, 2H), 7.78 (s, 1H), 7.67 (s, 2H), 7.52 (d, J=7.6 Hz, 2H), 7.34 (t, J=8.0 Hz, 2H), 7.06 (t, J=7.2 Hz, 1H), 3.09-3.16 (m, 2H), 0.99 (t, J=7.0 Hz, 3H) and the title product (24) as a white solid. MS (ESI): mass calcd, for C₁₉H₁₇F₃N₄O₃S: 438.10, found: 439.00 [M+H]⁺. ¹H NMR (400 MHz, DMSO-d6) δ ppm 10.98 (br s, 1H), 9.24 (s, 1H), 8.12-8.27 (m, 2H), 7.77 (s, 1H), 7.66 (s, 2H), 7.52 (d, J=7.6 Hz, 2H), 7.33 (t, J=8.0 Hz, 2H), 7.05 (t, J=7.2 Hz, 1H), 3.09-3.17 (m, 2H), 0.99 (t, J=7.2 Hz, 3H).

### Example 27. Ethyl 3,3,3-trifluoro-2-hydroxy-2-(2-(3-(pyridin-3-yl)ureido)benzo[d]thiazol-6-yl)propanoate (27)

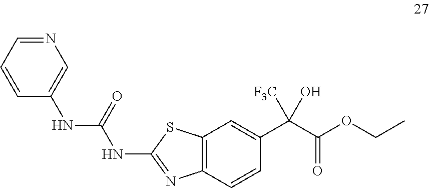 [SMILES: CCOC(=O)C(O)(c1ccc2nc(NC(=O)Nc3cccnc3)sc2c1)C(F)(F)F]

### Synthetic Scheme:

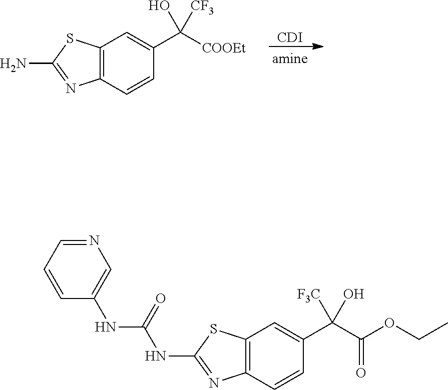 [SMILES: CCOC(=O)C(O)(c1ccc2nc(N)sc2c1)C(F)(F)F ; "CDI" ; CCOC(=O)C(O)(c1ccc2nc(NC(=O)Nc3cccnc3)sc2c1)C(F)(F)F]

### Ethyl 3,3,3-trifluoro-2-hydroxy-2-(2-(3-(pyridin-3-yl)ureido)benzo[d]thiazol-6-yl)propanoate

A solution of ethyl 2-(2-aminobenzo[d]thiazol-6-yl)-3,3,3-trifluoro-2-hydroxypropanoate (described in Example 6, 20 mg, 0.062 mmol) and CDI (12.2 mg, 74.9 μmol) in DMF (624 μL) was stirred at rt for 1 h. Pyridine-3-amine (6.37 μL, 0.075 mmol) and DIPEA (21.8 μL, 0.13 mmol) were added. After heating at 100° C. overnight, the reaction mixture was diluted with H₂O and directly purified by reverse phase HPLC (20-60% ACN/H₂O, with 0.1% TFA as a modifier) to afford the title compound (27) (6.3 mg, 23%, racemic) as a white solid. MS (ESI): mass calcd, for C₁₈H₁₅F₃N₄O₄S: 440.08, found: 440.95 [M+H]⁺. ¹H NMR (400 MHz, CDCl₃) δ ppm 11.24-11.32 (br s, 1H), 9.46 (s, 1H), 8.67 (d, J=8.1 Hz, 1H), 8.40 (d, J=5.4 Hz, 1H), 8.18 (s, 1H), 7.82 (dd, J=8.4, 5.3 Hz, 2H), 7.49-7.51 (m, 1H), 4.37-4.56 (m, 2H), 1.39 (t, J=7.1 Hz, 3H).

Examples 15 and 28 were synthesized in similar procedures as described in Example 27.

### Example 29. Ethyl 3,3,3-trifluoro-2-hydroxy-2-(2-(2-phenylacetamido)benzo[d]thiazol-6-yl)propanoate (29)

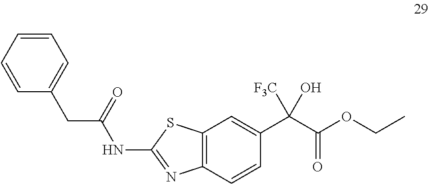 [SMILES: CCOC(=O)C(O)(c1ccc2nc(NC(=O)Cc3ccccc3)sc2c1)C(F)(F)F]

### Synthetic Scheme:

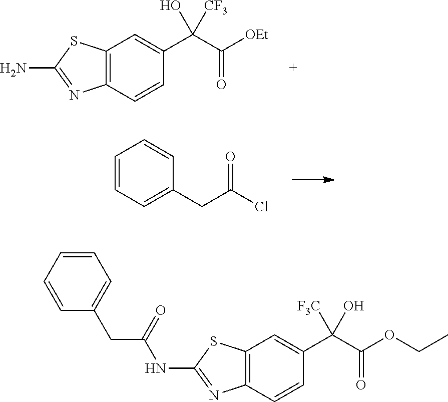

Ethyl 3,3,3-trifluoro-2-hydroxy-2-(2-(2-phenylacetamido)benzo[d]thiazol-6-yl)propanoate 2-Phenylacetyl chloride (11 μL, 0.081 mmol) was added to a solution of ethyl 2-(2-amino-1,3-benzothiazol-6-yl)-3,3,3-trifluoro-2-hydroxy-propanoate (20 mg, 0.062 mmol) in ACN (0.31 mL). After stirring at rt for 3 h, the reaction was quenched by sat. NH₄Cl. The aqueous layer was extracted with DCM (×3). The combined organic layer was dried over anhydrous Na₂SO₄, filtered, concentrated and purified by silica gel column chromatography (10-50% EtOAc/Hex) to afford the title product (29) (21.4 mg, 78.2%, racemic) as a white solid. MS (ESI): mass calcd, for C₂₀H₁₇F₃N₂O₄S: 438.09, found: 439.00 [M+H]⁺. ¹H NMR (400 MHz, CDCl₃) δ ppm 8.25 (s, 1H), 7.83 (d, J=8.7 Hz, 1H), 7.68 (d, J=8.7 Hz, 1H), 7.27-7.39 (m, 6H), 4.35-4.50 (m, 2H), 3.85 (s, 2H), 1.37 (t, J=7.1 Hz, 3H).

### Example 31. 3,3,3-Trifluoro-2-hydroxy-2-(2-(3-phenylureido)benzo[d]thiazol-6-yl)propenamide (31)

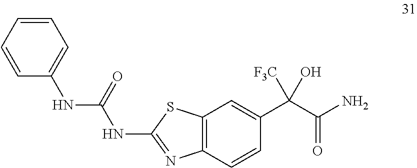 [SMILES: NC(=O)C(O)(c1ccc2nc(NC(=O)Nc3ccccc3)sc2c1)C(F)(F)F]

### Synthetic Scheme:

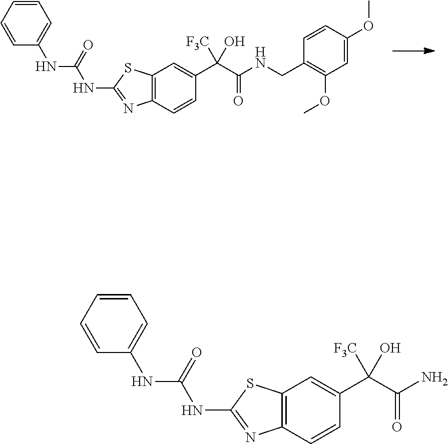 [SMILES: COc1ccc(CNC(=O)C(O)(c2ccc3nc(NC(=O)Nc4ccccc4)sc3c2)C(F)(F)F)c(OC)c1 ; NC(=O)C(O)(c1ccc2nc(NC(=O)Nc3ccccc3)sc2c1)C(F)(F)F]

A solution of N-(2,4-dimethoxybenzyl)-3,3,3-trifluoro-2-hydroxy-2-(2-(3-phenylureido)benzo[d]thiazol-6-yl)propanamide (Example 30, 1.5 mg, 2.7 μmol) in THE/DCM (1:1, 1 mL) was heated at 70° C. overnight. Upon completion, the reaction solvent was removed under reduced pressure. The resulting residue was purified by reverse phase HPLC (30-70% ACN/H₂O, with 0.05% NH₃HCO; as a modifier) to afford the title product (31) (0.38 mg, 34.6%, racemic) as a white solid. MS (BSI): mass calcd, for C₁₇H₁₃F₃N₄O₃S: 410.07, found: 410.95 [M+H]⁺. ¹H NMR (400 MHz, CDCl₃) δ ppm 8.18 (s, 1H), 7.83 (d, J=8.0 Hz, 1H), 7.76 (d, J=8.5 Hz, 1H), 7.52 (t, J=7.7 Hz, 2H), 7.36 (dd, J=14.2, 6.3 Hz, 2H), 7.12 (t, J=8.3 Hz, 1H), 6.36-6.44 (m, 3H).

### Example 34. 2-(2-((S)-2-Amino-2-phenylacetamido)benzo[d]thiazol-6-yl)-N-ethyl-3,3,3-trifluoro-2-hydroxypropanamide

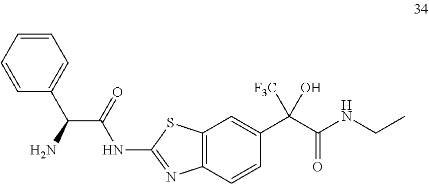 [SMILES: CCNC(=O)C(O)(c1ccc2nc(NC(=O)[C@@H](N)c3ccccc3)sc2c1)C(F)(F)F]

### Synthetic Scheme:

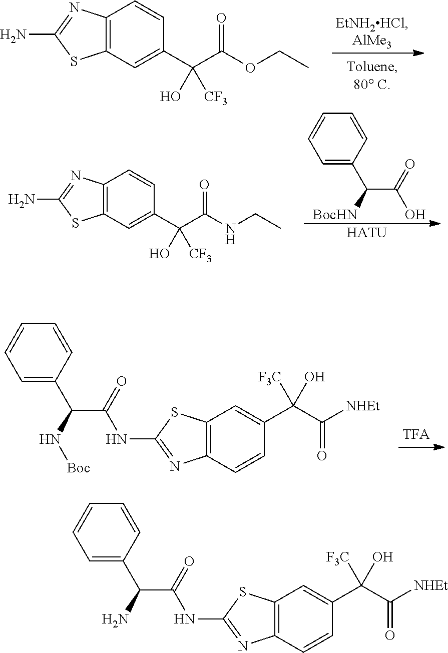

### 2-(2-Aminobenzo[d]thiazol-6-yl)-N-ethyl-3,3,3-trifluoro-2-hydroxypropanamide

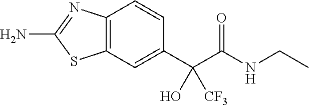 [SMILES: CCNC(=O)C(O)(c1ccc2nc(N)sc2c1)C(F)(F)F]

The title compound was prepared from ethyl 2-(2-aminobenzo[d]thiazol-6-yl)-3,3,3-trifluoro-2-hydroxypropanoate (described in Example 6) following Example 61, step 4 procedure.

### tert-Butyl ((1S)-2-((6-(3-(ethylamino)-1,1,1-trifluoro-2-hydroxy-3-oxopropan-2-yl)benzo[d]thiazol-2-yl)amino)-2-oxo-1-phenylethyl)carbamate

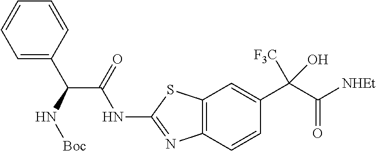

To a solution of 2-(2-aminobenzo[d]thiazol-6-yl)-N-ethyl-3,3,3-trifluoro-2-hydroxypropanamide (100 mg, 0.31 mmol) and (2S)-2-(tert-butoxycarbonylamino)-2-phenyl-acetic acid (78.7 mg, 0.31 mmol) in DMF (3 mL) was added HATU (178.6 mg, 0.47 mmol) and DIPEA (81 mg, 0.63 mmol). After stirring at 25° C. for 2 h, the reaction was diluted with H₂O) (5 mL) and extracted with EtOAc (5 mL×2). The organic layer was dried, filtered and concentrated under reduced pressure. The crude residue was purified by prep-TLC (SiO₂, PE:ethyl acetate=1:1) to provide the title product (131 mg, 75.7%) as a brown oil. MS (ESI): mass calcd, for C₂₅H₂₇F₃N₄O₅S: 552.17, found: 553.2 [M+H]⁺.

### 2-[2-[[(2S)-2-Amino-2-phenyl-acetyl]amino]-1,3-benzothiazol-6-yl]-N-ethyl-3,3,3-trifluoro-2-hydroxy-propanamide

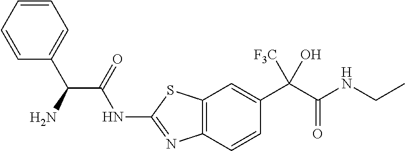 [SMILES: CCNC(=O)C(O)(c1ccc2nc(NC(=O)[C@@H](N)c3ccccc3)sc2c1)C(F)(F)F]

A solution of tert-butyl N-[(1S)-2-[[6-[1-(ethylcarbamoyl)-2,2,2-trifluoro-1-hydroxy-ethyl]-1,3-benzothiazol-2-yl]amino]-2-oxo-1-phenyl-ethyl]carbamate (118 mg, 0.21 mmol) in TFA (4.5 mL) and DCM (9 mL) was stirred at 25° C. for 2 h. The reaction was filtered and concentrated under reduced pressure. The resulting residue was purified by reverse phase HPLC (15-45% ACN in water (10 mM NH₄HCO₃), gradient separation) to provide the title product (34) (40 mg, 41.4%, racemic) as a white solid. MS (ESI): mass calcd. for C₂₀H₁₉F₃N₄O₃S: 452.11, found 453.1 [M+H]⁺. ¹H NMR (400 MHz, DMSO-d₆) δ ppm 8.18-8.21 (m, 2H), 7.69-7.73 (m, 1H), 7.68 (d, J=5.6 Hz, 1H), 7.50 (d, J=7.6 Hz, 2H), 7.35-7.38 (m, 2H), 7.27-7.32 (m, 1H), 6.09-6.45 (m, 2H), 4.75 (s, 1H), 3.09-3.15 (m, 2H), 0.96-1.01 (m, 3H).

Example 35 was synthesized in similar procedures as described in Example 34.

### Example 36, N-Ethyl-3,3,3-trifluoro-2-(2-(3-(4-fluorophenyl)ureido)benzo[d]thiazol-6-yl)-2-hydroxypropanamide (36)

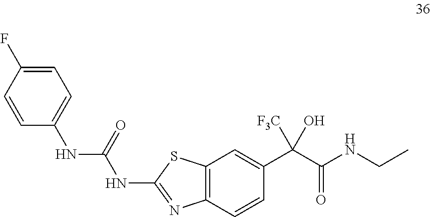 [SMILES: CCNC(=O)C(O)(c1ccc2nc(NC(=O)Nc3ccc(F)cc3)sc2c1)C(F)(F)F]

### Synthetic Scheme:

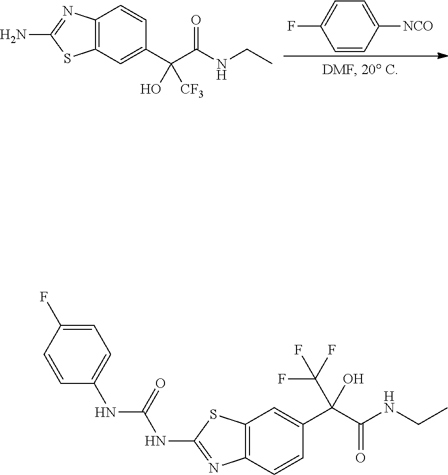

### N-Ethyl-3,3,3-trifluoro-2-(2-(3-(4-fluorophenyl)ureido)benzo[d]thiazol-6-yl)-2-hydroxypropanamide

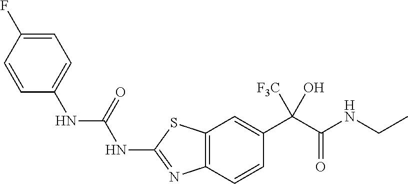 [SMILES: CCNC(=O)C(O)(c1ccc2nc(NC(=O)Nc3ccc(F)cc3)sc2c1)C(F)(F)F]

To a solution of 2-(2-aminobenzo[d]thiazol-6-yl)-N-ethyl-3,3,3-trifluoro-2-hydroxypropanamide (described in Example 34, 0.1 g, 0.31 mmol) in DMF (4 mL) was added 4-fluorophenyl isocyanate (51.5 mg, 0.37 mmol). After stirring at 20° C. for 12 h, the reaction was quenched by saturated NH₄Cl (3 mL), and extracted with EtOAc (5 mL×3). The combined organic layer was washed with brine, dried over Na₂SO₄, filtered and concentrated under reduced pressure. The residue was purified by prep-TLC (SiO₂, PE:ethyl acetate=1:1) to provide the title product (36) (3 mg, 2.1%, racemic) as a white solid. MS (ESI): mass calcd, for C₁₉H₁₆F₄N₄O₃S: 456.09, found: 457.1 [M+H]⁺. ¹H NMR (400 MHz, DMSO-d₆) δ ppm 10.96 (br s, 1H), 9.29 (br s, 1H), 8.13-8.27 (m, 2H), 7.80 (s, 1H), 7.66 (br s, 2H), 7.50-7.59 (m, 2H), 7.13-7.21 (m, 2H), 3.08-3.16 (m, 2H), 0.99 (t, J=7.2 Hz, 3H).

Examples 53, and 55 were synthesized in similar procedures as described in Example 36.

Example 37 was prepared by chiral SFC separation of Example 36 (column: ChiralPak IH, 250*30 mm, 10 μm; mobile phase: 17-45% gradient i-PrOH with 0.1% NH₃H₂O. Example 37 is the first eluting peak from chiral separation.

### Example 38. (S)—N-Ethyl-3,3,3-trifluoro-2-(2-(3-(4-fluorophenyl)ureido)benzo[d]thiazol-6-yl)-2-hydroxypropanamide

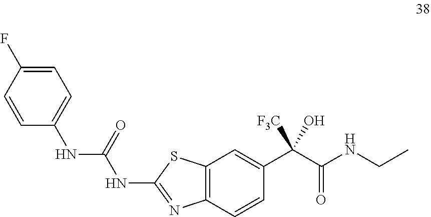 [SMILES: CCNC(=O)[C@@](O)(c1ccc2nc(NC(=O)Nc3ccc(F)cc3)sc2c1)C(F)(F)F]

### Synthetic Scheme:

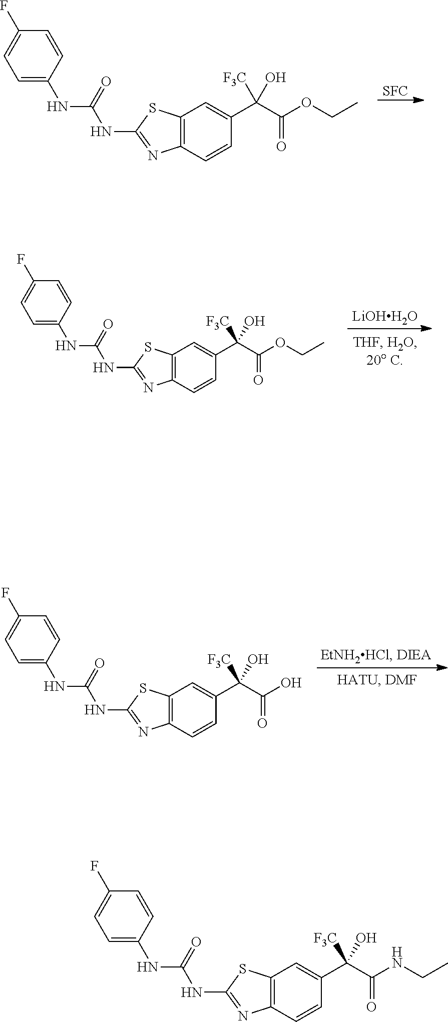

### Ethyl (S)-3,3,3-trifluoro-2-(2-(3-(4-fluorophenyl)ureido)benzo[d]thiazol-6-yl)-2-hydroxypropanoate

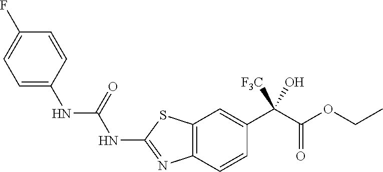 [SMILES: CCOC(=O)[C@@](O)(c1ccc2nc(NC(=O)Nc3ccc(F)cc3)sc2c1)C(F)(F)F]

The title compound was obtained as the first eluting peak through chiral SFC separation of ethyl 3,3,3-trifluoro-2-(2-(3-(4-fluorophenyl)ureido)benzo[d]thiazol-6-yl)-2-hydroxypropanoate (Example 21), with DAICEL CHIRALPAK AD (250 mm*50 mm, 10 um; mobile phase: 40% EtOH with 0.1% NH₃H₂O) to provide the title compound (1.1 g, 30.6%) as a white solid. MS (ESI): mass calcd, for C₁₉H₁₅F₄N₅O₄S: 457.07, found: 458.1 [M+H]⁺.

### (S)-3,3,3-Trifluoro-2-(2-(3-(4-fluorophenyl)ureido)benzo[d]thiazol-6-yl)-2-hydroxypropanoic Acid

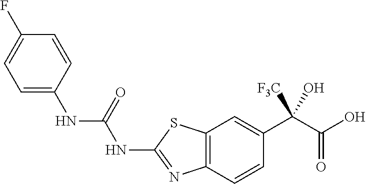 [SMILES: O=C(Nc1ccc(F)cc1)Nc1nc2ccc([C@](O)(C(=O)O)C(F)(F)F)cc2s1]

To a solution of ethyl (S)-3,3,3-trifluoro-2-(2-(3-(4-fluorophenyl)ureido)benzo[d]thiazol-6-yl)-2-hydroxypropanoate (1.1 g, 2.4 mmol) in THF (16 mL) and H₂O (3 mL) was added LiOH·H₂O (1.05 g, 24.9 mmol) in one portion at 25° C. After stirring at rt for 3 h, the reaction was quenched by addition of aqueous HCl (1 N) to adjust to pH=3. The resulting mixture was extracted with EtOAc (10 mL×2). The combined organic layer was washed with brine, dried over Na₂SO₄, filtered and concentrated under reduced pressure to afford the title product (0.93 g, 90.1%) as a white solid, which was directly used in the next step without further purification. MS (ESI); mass calcd. for C₁₇H₁₁F₄N₃O₄S: 429.04, found: 430.1 [M+H]⁻.

### (S)—N-Ethyl-3,3,3-trifluoro-2-(2-(3-(4-fluorophenyl)ureido)benzo[d]thiazol-6-yl)-2-hydroxypropanamide

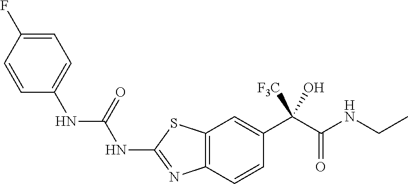 [SMILES: CCNC(=O)[C@@](O)(c1ccc2nc(NC(=O)Nc3ccc(F)cc3)sc2c1)C(F)(F)F]

To an ice cold solution of (S)-3,3,3-trifluoro-2-(2-(3-(4-fluorophenyl)ureido)benzo[d]thiazol-6-yl)-2-hydroxypropanoic acid (650 mg, 1.51 mmol) and ethylamine hydrochloride (234 mg, 2.88 mmol) in DMF (7 mL) was added DIPEA (1.32 mL, 7.57 mmol) and HATU (1.73 g, 4.54 mmol) under N₂. It was allowed to stir at rt for 8 h. Upon completion, the reaction was quenched with H₂O) (20 mL). The aqueous layer was extracted with EtOAc (20 mL×3). The combined organic layer was washed with brine, dried over anhydrous Na₂SO₄, filtered, concentrated and purified by reverse phase HPLC (gradient elution, 30-50% ACN in H₂O, with 0.05% NH₄HCO₃ as a modifier) to afford the title product (38) (142 mg, 20.6%) as a white solid. MS (ESI): mass calcd, for C₁₉H₁₆F₄N₄O₃S: 456.09, found: 457.1 [M+H]⁺. ¹H NMR (400 MHz, DMSO-d₆) δ ppm 10.90 (br s, 1H), 9.20 (br s, 1H), 8.23 (t, J=5.8 Hz, 1H), 8.17 (br s, 1H), 7.79 (s, 1H), 7.67 (br s, 2H), 7.54 (br s, 2H), 7.18 (t, J=8.6 Hz, 2H), 3.07-3.17 (m, 2H), 0.99 (t, J=7.1 Hz, 3H).

### Example 39, 1-(6-(4-Methyl-2,5-dioxoimidazolidin-4-yl)benzo[d]thiazol-2-yl)-3-phenylurea (39)

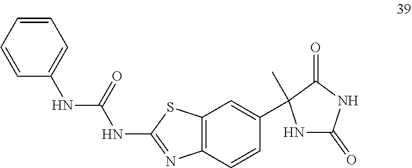 [SMILES: CC1(c2ccc3nc(NC(=O)Nc4ccccc4)sc3c2)NC(=O)NC1=O]

### Synthetic Scheme:

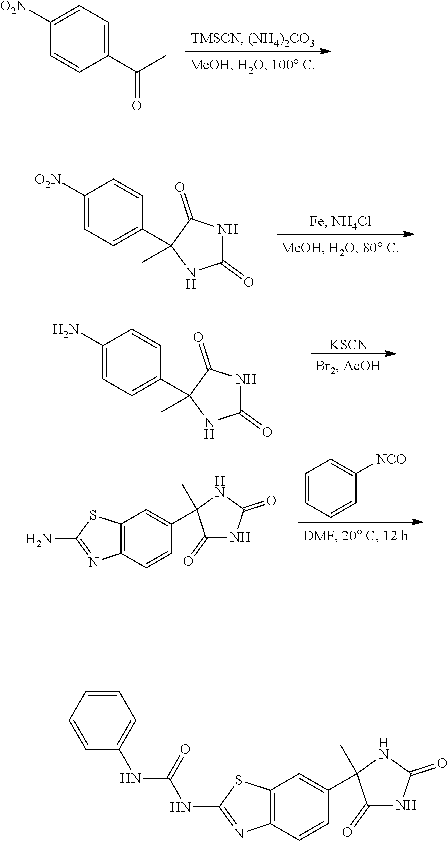

### 5-Methyl-5-(4-nitrophenyl) imidazolidine-2,4-dione

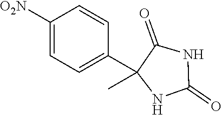 [SMILES: CC1(c2ccc([N+](=O)[O-])cc2)NC(=O)NC1=O]

To a solution of 1-(4-nitrophenyl) ethanone (5 g, 30.3 mmol) in MeOH (25 mL) and H₂O (25 mL) was added TMSCN (11.4 mL, 90.8 mmol) and (NH₄)₂CO₃ (11.64 g, 121.1 mmol). After heating at 90° C. for 3 h, the reaction mixture was filtered, and the filtrate was concentrated under reduced pressure. The crude material was suspended in EtOAc (20 mL) and stirred for 10 min to form a precipitation. The precipitate was filtered and dried to afford the title product (6 g, 84.3%) as a yellow solid. MS (ESI): mass calcd, for C₁₀H₉N₃O₄: 235.06, found: 236.1 [M+H]⁺.

### 5-(4-Aminophenyl)-S-methylimidazolidine-2,4-dione

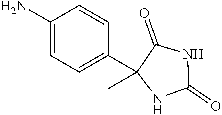

To a solution of 5-methyl-5-(4-nitrophenyl) imidazolidine-2,4-dione (5 g, 21.3 mmol) in MeOH (30 mL) and H₂O (30 mL) was added NH₄Cl (11.37 g, 212.6 mmol) and Fe (11.87 g, 212.6 mmol). After heating at 80° C. for 4 h, the reaction was partitioned between H₂O (50 mL) and EtOAc (100 mL). The aqueous layer was extracted with EtOAc (100 mL×2). The combined organic layer was washed with brine, dried over Na₂SO₄, filtered, and concentrated under reduced pressure. The crude material was suspended in EtOAc (20 mL) and stirred for 10 min to form a precipitation. The precipitate was filtered and dried to afford the title product (4.11 g, 94.2%) as a yellow solid. MS (ESI): mass calcd, for C₁₀H₁₁N₃O₂: 205.09, found: 206.1 [M+H]⁺.

### 5-(2-Aminobenzo[d]thiazol-6-yl)-S-methylimidazolidine-2,4-dione

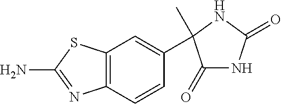 [SMILES: CC1(c2ccc3nc(N)sc3c2)NC(=O)NC1=O]

To a solution of 5-(4-aminophenyl)-5-methyl-imidazolidine-2,4-dione (2 g, 9.8 mmol) in AcOH (60 mL) was added KSCN (3.31 g, 34.1 mmol). After stirring at 25° C. for 2 h, a solution of Br₂ (0.55 mL, 10.7 mmol) in AcOH (10 mL) was added. The reaction mixture was stirred at 25° C. for 3 h. After completion, the solution was filtered, and the filter cake was washed with EtOAc (20 mL×3). The filtrate was concentrated under reduced pressure. The resulting residue was treated with sat. NaHCO₃ (50 mL) to neutralize the remaining AcOH. The aqueous solution was extracted with EtOAc (50 mL×2). The combined organic layer was washed with brine (20 mL), dried over anhydrous Na₂SO₄, filtered, concentrated, and purified by silica gel column chromatography (0-100% EtOAc/PE) to afford the title product (2.31 g, 90.4%) as a yellow solid. MS (ESI): mass calcd, for C₁₁H₁₀N₄O₂S: 262.05, found: 263.0 [M+H]⁺.

### 1-(6-(4-Methyl-2,5-dioxoimidazolidin-4-yl)benzo[d]thiazol-2-yl)-3-phenylurea

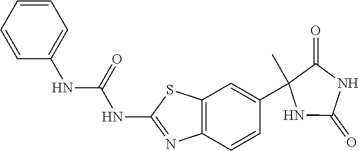 [SMILES: CC1(c2ccc3nc(NC(=O)Nc4ccccc4)sc3c2)NC(=O)NC1=O]

To a solution of 5-(2-aminobenzo[d]thiazol-6-yl)-5-methylimidazolidine-2,4-dione (200 mg, 0.76 mmol) in DMF (3 mL) was added phenyl isocyanate (136.3 μL. 1.14 mmol). After stirring at rt for 12 h, the reaction mixture was quenched with H₂O (30 mL). The aqueous layer was extracted with EtOAc (100 mL×3). The combined organic layer was washed with brine (20 mL×3), dried over anhydrous Na₂SO₄, filtered and concentrated under reduced pressure. The crude material was suspended in EtOAc (5 mL) and stirred for 10 min to form a precipitation. The precipitate was filtered and dried to afford the title product (39) (7.7 g, 15.4%, racemic) as a white solid. MS (ESI): mass calcd. for C₁₈H₁₅N₅O₃S: 381.09, found: 382.0 [M+H]⁺. ¹H NMR (400 MHz, DMSO-d6) δ ppm 10.78 (br s, 2H), 9.18 (br s, 1H), 8.64 (s, 1H), 8.01 (s, 1H), 7.65 (br s, 1H), 7.51 (d, J=8.0 Hz, 2H), 7.45-7.47 (m, 1H), 7.33 (t, J=8.0 Hz, 2H), 7.33 (t, J=7.2 Hz, 1H), 1.70 (s, 3H).

### Example 40. Ethyl 3,3,3-trifluoro-2-hydroxy-2-(2-(3-phenylureido) imidazo[1,2-a]pyridin-6-yl)propanoate (40)

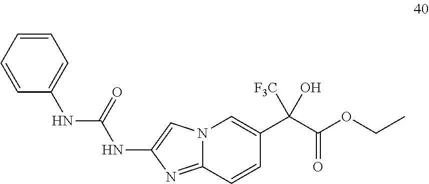 [SMILES: CCOC(=O)C(O)(c1ccc2nc(NC(=O)Nc3ccccc3)cn2c1)C(F)(F)F]

### Synthetic Scheme:

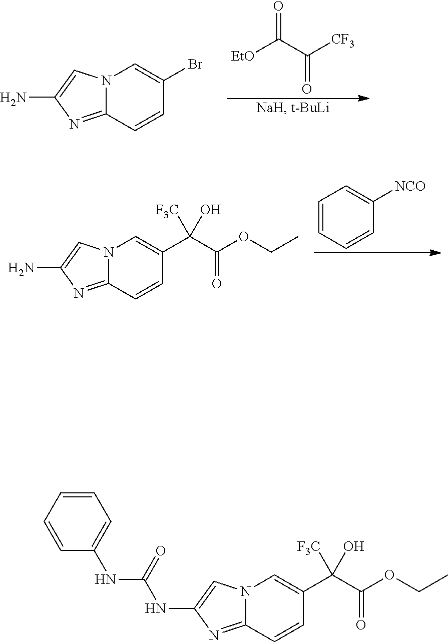

### Ethyl 2-(2-aminoimidazo[1,2-a]pyridin-6-yl)-3,3,3-trifluoro-2-hydroxypropanoate

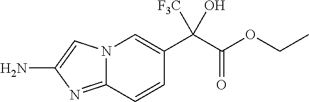 [SMILES: CCOC(=O)C(O)(c1ccc2nc(N)cn2c1)C(F)(F)F]

To an ice cold solution of 6-bromoimidazo[1,2-a]pyridin-2-amine (500 mg, 2.36 mmol) in THF (15 mL) was added 60% NaH (188.6 mg, 4.72 mmol) under N₂. It was allowed to stir at 0° C. for 30 min. Then a solution of 1.3 M t-BuLi in heptane (4.5 mL, 5.89 mmol) was added dropwise at −70° C. After stirring at this temperature for 30 min, ethyl 3,3,3-trifluoro-2-oxo-propanoate (0.47 mL, 3.54 mmol) was added. The reaction was stirred for another 30 min, quenched with sat. NH₄Cl (10 mL) and extracted with EtOAc (10 mL×3). The combined organic layer was washed with brine (5 mL×2), dried over anhydrous Na₂SO₄, filtered, concentrated and purified by silica gel chromatography (25-50% EtOAc/PE) to afford the title product (180 mg, 31.5%) as a brown solid. MS (ESI): mass calcd, for C₁₂H₁₂F₃N₃O₃: 303.08, found: 304.0 [M+H]⁺.

### Ethyl 3,3,3-trifluoro-2-hydroxy-2-(2-(3-phenylureido) imidazo[1,2-a]pyridin-6-yl)propanoate

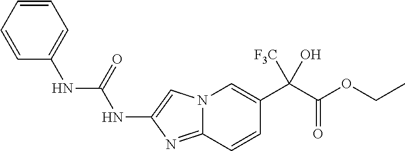 [SMILES: CCOC(=O)C(O)(c1ccc2nc(NC(=O)Nc3ccccc3)cn2c1)C(F)(F)F]

The title compound (40) (racemic) was synthesized from 6-bromoimidazo[1,2-a]pyridin-2-amine in similar procedures as described in Example 1. MS (ESI): mass calcd. for C₁₉H₁₇F₃N₄O₄: 422.12, found: 423.1 [M+H]⁺. ¹H NMR (400 MHz, DMSO-d6) δ ppm 9.78 (s, 1H), 9.01 (s, 1H), 8.46 (d, J=7.2 Hz, 1H), 7.83 (s, 1H), 7.66 (d, J=8.8 Hz, 1H), 7.50 (d, J=8.0 Hz, 2H), 7.38-7.46 (m, 1H), 7.30 (t, J=7.6 Hz, 2H), 7.06 (t, J=6.8 Hz, 1H), 7.00 (t, J=7.2 Hz, 1H), 4.22-4.26 (m, 2H), 1.17 (t, J=7.2 Hz, 3H).

### Example 41. 1-(6-(2,5-Dioxo-4-(trifluoromethyl) imidazolidin-4-yl)benzo[d]thiazol-2-yl)-3-phenylurea (41)

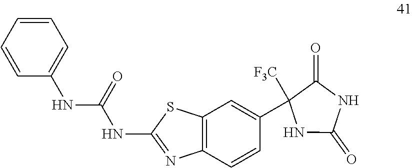 [SMILES: O=C(Nc1ccccc1)Nc1nc2ccc(C3(C(F)(F)F)NC(=O)NC3=O)cc2s1]

### Synthetic Scheme:

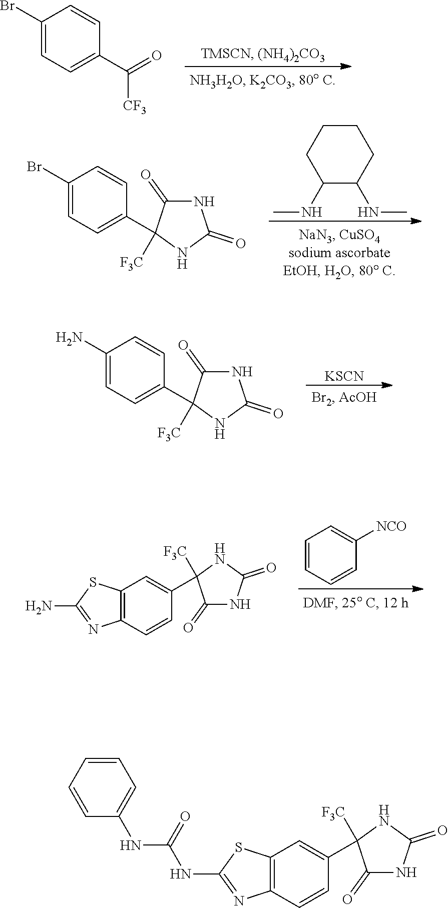

### S-(4-Bromophenyl)-S-(trifluoromethyl) imidazolidine-2,4-dione

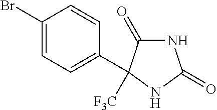 [SMILES: O=C1NC(=O)C(c2ccc(Br)cc2)(C(F)(F)F)N1]

To a solution of 1-(4-bromophenyl)-2,2,2-trifluoro-ethanone (10 g, 39.52 mmol) in NH₃H₂O) (50 mL) and H₂O (50 mL) was added (NH₄)₂CO₃ (30.38 g, 316.2 mmol), K₂CO₃, (32.78 g, 237.1 mmol), and TMSCN (19.78 mL, 158.1 mmol). After heating at 80° C. for 12 h, the reaction mixture was cooled to rt and diluted with H₂O (50 mL). The aqueous layer was extracted with EtOAc (50 mL×3). The combined organic layer was washed with brine (50 mL), dried over anhydrous Na₂SO₄, filtered, concentrated, and purified by silica gel column chromatography (1-60% EtOAc/PE) and prep-TLC (33% EtOAc/PE) to afford the title product (0.49 g, 51.4%) as a yellow solid. MS (ESI): mass calcd, for C₁₀H₆BrF₃N₂O₂: 321.96, found: 323.0 [M+H]⁺.

### 5-(4-Aminophenyl)-S-(trifluoromethyl) imidazolidine-2,4-dione

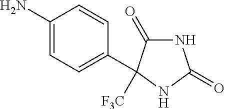 [SMILES: Nc1ccc(C2(C(F)(F)F)NC(=O)NC2=O)cc1]

To a solution of 5-(4-bromophenyl)-5-(trifluoromethyl) imidazolidine-2,4-dione (0.618 g, 1.91 mmol) and NaN₃ (0.49 g, 7.6 mmol) in EtOH (5 mL) and H₂O (1 mL) was added CuSO₄ (0.30 g, 1.91 mmol). N1,N2-dimethylcyclohexane-1,2-diamine (0.16 g, 1.15 mmol) and sodium ascorbate (0.76 g, 3.82 mmol) in one portion at rt. After heating at 80° C. for 3 h, the reaction mixture was diluted with water (5 mL). The aqueous layer was extracted with EtOAc (5 mL×3). The combined organic layer was washed with brine (5 mL), dried over anhydrous Na₂SO₄, filtered, concentrated and purified by prep-TLC (50% EtOAc/PE) to afford the title product (0.18 g, 35.9%) as a yellow solid. MS (ESI): mass calcd. for C₁₀H₈F₃N₃O₂: 259.06, found: 258.2 [M−H]⁻.

### 5-(2-Amino-1,3-benzothiazol-6-yl)-5-(trifluoromethyl) imidazolidine-2,4-dione

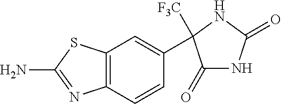 [SMILES: Nc1nc2ccc(C3(C(F)(F)F)NC(=O)NC3=O)cc2s1]

To a solution of 5-(4-aminophenyl)-5-(trifluoromethyl) imidazolidine-2,4-dione (0.18 g, 0.69 mmol) in AcOH (1 mL) was added KSCN (223 mg, 2.3 mmol). After stirring at 25° C. for 2 h, a solution of Br₂ (37 μL, 0.72 mmol) in AcOH (1 mL) was added. The reaction mixture was stirred at 25° C. for another 12 h. After completion, the solution was filtered, and the filter cake was washed with EtOAc (10 mL×3). The filtrate was concentrated under reduced pressure. The resulting residue was treated with sat. NaHCO₃ (20 mL) to neutralize the remaining AcOH. The aqueous solution was extracted with ethyl acetate (20 mL×2). The combined organic layer was washed with brine (5 mL), dried over anhydrous Na₂SO₄, filtered, and concentrated. The resulting residue was triturated with MTBE to obtain the crude title product (0.2 g, 80% purity) as a yellow solid. MS (ESI): mass calcd. for C₁₁H₇F₃N₄O₂S: 316.02, found: 317.1 [M+H]⁺.

### 1-(6-(2,5-Dioxo-4-(trifluoromethyl) imidazolidin-4-yl)benzo[d]thiazol-2-yl)-3-phenylurea

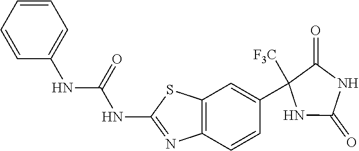 [SMILES: O=C(Nc1ccccc1)Nc1nc2ccc(C3(C(F)(F)F)NC(=O)NC3=O)cc2s1]

To a solution of 5-(2-amino-1,3-benzothiazol-6-yl)-5-(trifluoromethyl) imidazolidine-2,4-dione (180 mg, 0.59 mmol) in DMF (2 mL) was added phenyl isocyanate (127.7 μL, 1.2 mmol). After stirring at rt for 12 h, the reaction mixture was adjusted to pH=6 with citric acid. The aqueous layer was extracted with EtOAc (10 mL×2). The combined organic layer was washed with brine (2 mL×3), dried over anhydrous Na₂SO₄, filtered, concentrated, and purified by reverse phase HPLC (gradient elution, 10-45% ACN in H₂O, with 0.05% NH₄HCO₃ as a modifier) to afford the title product (41) (5.3 mg, 10.5%, racemic) as a white solid. MS (ESI): mass calcd, for C₁₈H₁₂F₃N₅O₃S: 435.06, found: 434.0 [M−H]. 1H NMR (400 MHz, DMSO-d6) δ ppm 10.53 (br s, 1H), 7.79 (br s, 1H), 7.57-7.65 (m, 2H), 7.40 (d, J=7.8 Hz, 2H), 7.24-7.37 (m, 4H), 7.07-7.20 (m, 1H), 6.93-7.07 (m, 1H).

### Example 47. N-Ethyl-3,3,3-trifluoro-2-hydroxy-2-(2-(3-phenylureido)quinolin-6-yl)propenamide (47)

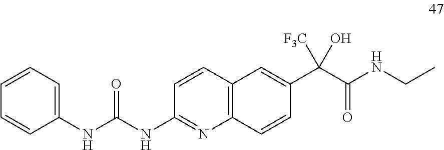 [SMILES: CCNC(=O)C(O)(c1ccc2nc(NC(=O)Nc3ccccc3)ccc2c1)C(F)(F)F]

### Synthetic Scheme:

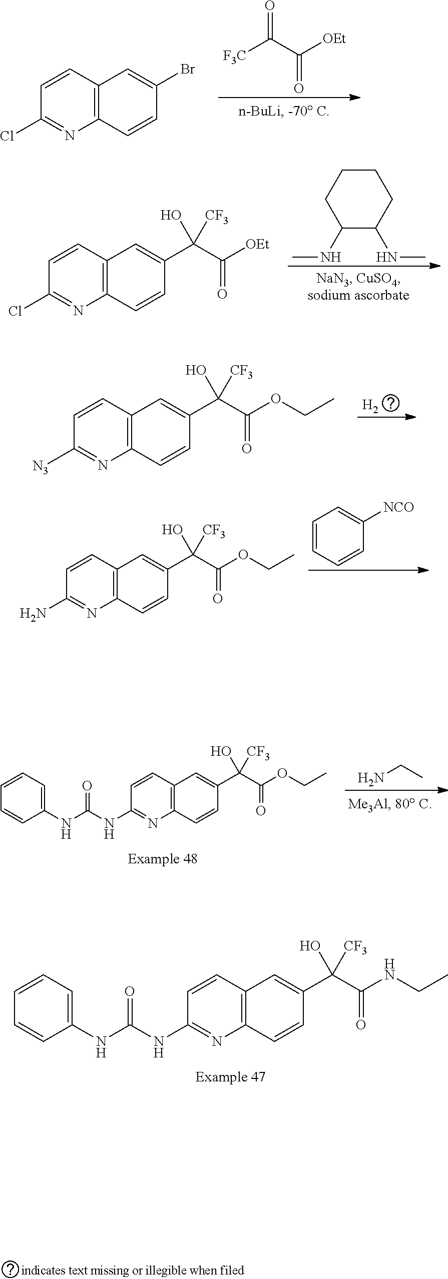

### Ethyl 3,3,3-trifluoro-2-hydroxy-2-(2-(3-phenylureido)quinolin-6-yl)propanoate (Example 48)

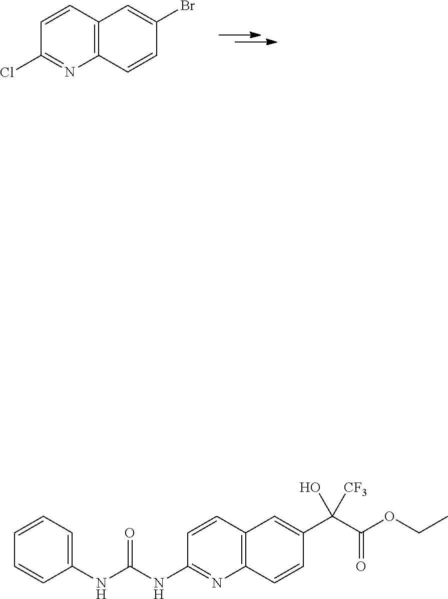 [SMILES: Clc1ccc2cc(Br)ccc2n1 ; CCOC(=O)C(O)(c1ccc2nc(NC(=O)Nc3ccccc3)ccc2c1)C(F)(F)F]

The title compound (48) was synthesized from 1-(tert-butyl) 2-ethyl 2-(2-(3-(4-fluorophenyl)ureido)benzo[d]thiazol-6-yl)pyrrolidine-1,2-dicarboxylate in similar procedures as described in Example 73. MS (ESI): mass calcd, for C₂₁H₁₈F₃N₃O₄: 433.12, found: 434.2 [M+H]⁺. ¹H NMR (400 MHz, DMSO-d₆) δ ppm 11.85 (s, 1H), 10.14 (s, 1H), 8.42 (d, J=8.8 Hz, 1H), 8.10 (d, J=1.6 Hz, 1H), 8.05 (s, 1H), 8.00 (d, J=8.8 Hz, 1H), 7.82-7.88 (m, 1H), 7.67 (d, J=7.6 Hz, 2H), 7.44 (d, J=8.8 Hz, 1H), 7.36 (t, J=8.0 Hz, 2H), 7.08 (t, J=7.6 Hz, 1H), 4.25-4.36 (m, 2H), 1.23 (t, J=7.2 Hz, 3H).

### N-Ethyl-3,3,3-trifluoro-2-hydroxy-2-(2-(3-phenylureido)quinolin-6-yl)propenamide

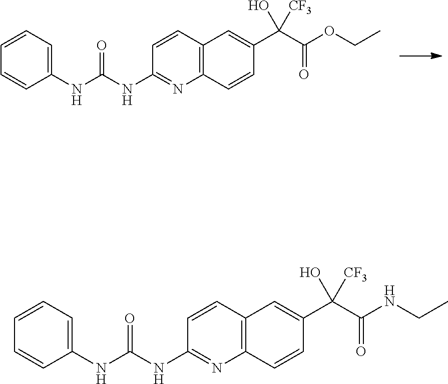 [SMILES: CCOC(=O)C(O)(c1ccc2nc(NC(=O)Nc3ccccc3)ccc2c1)C(F)(F)F ; CCNC(=O)C(O)(c1ccc2nc(NC(=O)Nc3ccccc3)ccc2c1)C(F)(F)F]

The title compound (47) was synthesized from ethyl 3,3,3-trifluoro-2-hydroxy-2-(2-(3-phenylureido)quinolin-6-yl)propanoate in similar procedures as described in Example 13. MS (ESI): mass calcd, for C₂₁H₁₉F₃N₄O₃: 432.14, found: 433.1 [M+H]⁺. ¹H NMR (400 MHz, DMSO-d6) δ ppm 11.88 (s, 1H), 10.12 (s, 1H), 8.38 (d, J=8.8 Hz, 1H), 8.29 (t, J=6.0 Hz, 1H), 8.15 (s, 1H), 7.90-7.99 (m, 3H), 7.68 (d, J=7.6 Hz, 2H), 7.42 (d, J=8.8 Hz, 1H), 7.36 (t, J=8.0 Hz, 2H), 7.08 (t, J=7.2 Hz, 1H), 3.12-3.17 (m, 2H), 0.99 (t, J=7.2 Hz, 3H)

### Example 52. 3,3,3-Trifluoro-2-(2-(3-(4-fluorophenyl)ureido)benzo[d]thiazol-6-yl)-2-hydroxy-N-(oxetan-3-yl)propenamide (52)

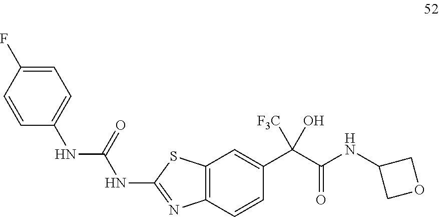 [SMILES: O=C(Nc1ccc(F)cc1)Nc1nc2ccc(C(O)(C(=O)NC3COC3)C(F)(F)F)cc2s1]

### Synthetic Scheme:

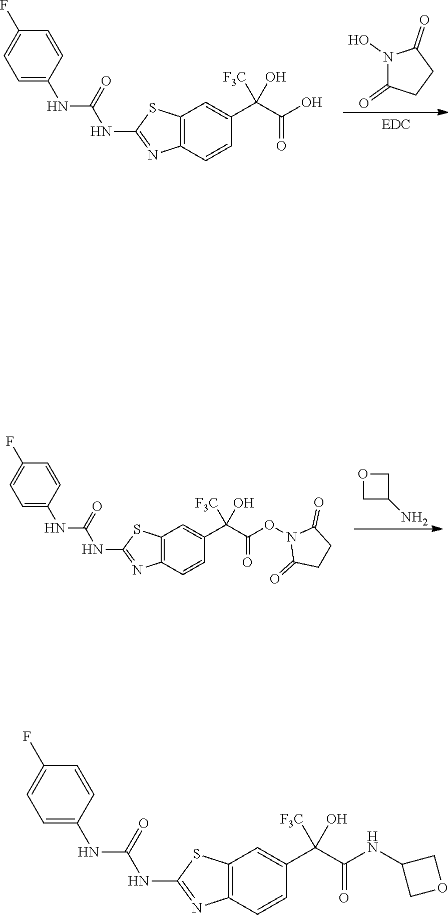 [SMILES: O=C(Nc1ccc(F)cc1)Nc1nc2ccc(C(O)(C(=O)O)C(F)(F)F)cc2s1 ; O=C1CCC(=O)N1O ; O=C(Nc1ccc(F)cc1)Nc1nc2ccc(C(O)(C(=O)ON3C(=O)CCC3=O)C(F)(F)F)cc2s1 ; NC1COC1 ; O=C(Nc1ccc(F)cc1)Nc1nc2ccc(C(O)(C(=O)NC3COC3)C(F)(F)F)cc2s1]

### 2,5-Dioxopyrrolidin-1-yl 3,3,3-trifluoro-2-(2-(3-(4-fluorophenyl)ureido)benzo[d]thiazol-6-yl)-2-hydroxypropanoate

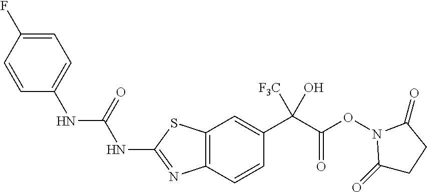 [SMILES: O=C(Nc1ccc(F)cc1)Nc1nc2ccc(C(O)(C(=O)ON3C(=O)CCC3=O)C(F)(F)F)cc2s1]

A solution of 3,3,3-trifluoro-2-(2-(3-(4-fluorophenyl)ureido)benzo[d]thiazol-6-yl)-2-hydroxypropanoic acid (synthesized similarly as Example 10, 53 mg, 0.12 mmol), N-hydroxysuccinimide (21.3 mg, 0.19 mmol), EDC (35.5 mg, 0.19 mmol) and DMAP (1.51 mg, 0.012 mmol) in DMF (1.2 mL) was stirred at rt. After 4 h, the reaction mixture was diluted with EtOAc and washed with brine (×3). The organic layer was dried over anhydrous Na₂SO₄, filtered, and concentrated to afford the crude title product (65 mg) as a yellow oil, which was directly used in the next step without further purification. MS (ESI): mass calcd. for C₂₁H₁₄F₄N₄O₆S: 526.06, found: 526.95 [M+H]⁺.

### 3,3,3-Trifluoro-2-(2-(3-(4-fluorophenyl)ureido)benzo[d]thiazol-6-yl)-2-hydroxy-N-(oxetan-3-yl)propanamide

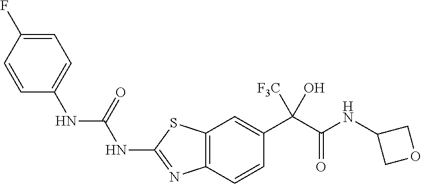 [SMILES: O=C(Nc1ccc(F)cc1)Nc1nc2ccc(C(O)(C(=O)NC3COC3)C(F)(F)F)cc2s1]

Oxetan-3-amine (17.3 μL, 0.25 mmol) and TEA (52 μL, 0.37 mmol) were added to a solution of 2,5-dioxopyrrolidin-1-yl-3-(2-(3-(4-fluorophenyl)ureido)benzo[d]thiazol-6-yl)tetrahydrofuran-3-carboxylate (65 mg, 0.12 mmol) in THF (1.23 mL). The resulting solution was stirred at rt. Upon completion, the reaction solvent was removed under reduced pressure and the resulting residue was purified by reverse phase HPLC (35-75% ACN/H₂O, with 0.1% TFA as a modifier) to afford the title product (52) (44.1 mg, 73.8%, racemic) as a white solid. MS (ESI): mass calcd, for C₂₀H₁₆F₄N₄O₄S: 484.08, found: 484.95 [M+H]⁺. ¹H NMR (400 MHz, CDCl₃) δ ppm 8.11 (s, 1H), 7.80 (d, J=8.7 Hz, 1H), 7.72 (d, J=7.9 Hz, 1H), 7.52 (dd, J=8.9, 4.6 Hz, 2H), 7.10 (t, J=8.6 Hz, 2H), 6.88 (d, J=6.8 Hz, 1H), 5.05 (dt, J=13.6, 6.5 Hz, 1H), 4.95 (td, J=7.1, 1.4 Hz, 2H), 4.48 (dt, J=11.6, 6.0 Hz, 2H).

Examples 25, 26, 30, 32, 33, 57, 58, and 59 were synthesized in similar procedures as described in Example 52.

### Example 56. Ethyl 3,3,3-trifluoro-2-hydroxy-2-(1-methyl-2-(3-phenylureido)-1H-benzo[d]imidazol-6-yl)propanoate (56)

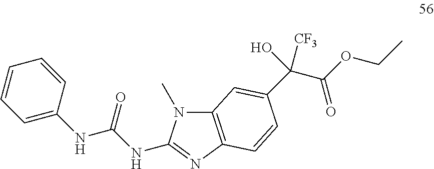 [SMILES: CCOC(=O)C(O)(c1ccc2nc(NC(=O)Nc3ccccc3)n(C)c2c1)C(F)(F)F]

### Synthetic Scheme:

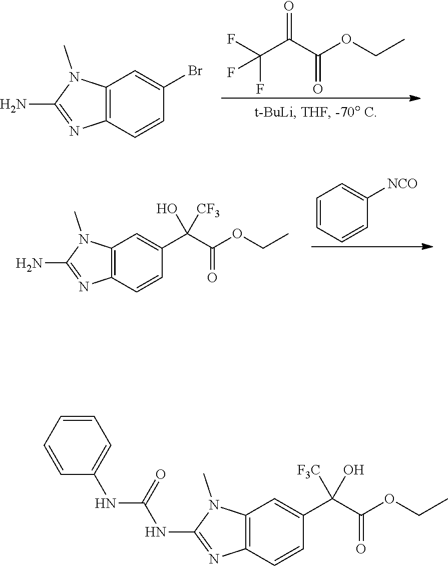

### Ethyl 3,3,3-trifluoro-2-hydroxy-2-(1-methyl-2-(3-phenylureido)-1H-benzo[d]imidazol-6-yl)propanoate

The title compound (56) was synthesized from 6-bromo-1-methyl-1H-benzo[d]imidazol-2-amine in similar procedures as described in Example 40. MS (ESI): mass calcd, for C₂₀H₁₉F₃N₄O₄: 436.14, found: 437.3 [M+H]⁺. ¹H NMR (400 MHz, DMSO-d6) δ ppm 12.23 (s, 1H), 9.12 (s, 1H), 7.85 (s, 1H), 7.63-7.70 (m, 2H), 7.40-7.46 (m, 2H), 7.23-7.30 (m, 3H), 6.90 (br s, 1H), 4.22-4.30 (m, 2H), 3.55 (s, 3H), 0.99 (t, J=7.2 Hz, 3H).

### Example 60, Ethyl 3,3,3-trifluoro-2-hydroxy-2-(2-(3-phenylureido)benzo[d]oxazol-6-yl)propanoate (60)

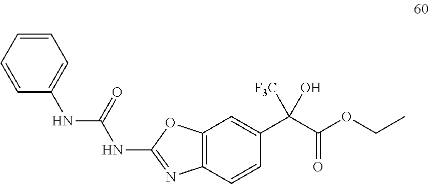 [SMILES: CCOC(=O)C(O)(c1ccc2nc(NC(=O)Nc3ccccc3)oc2c1)C(F)(F)F]

### Synthetic Scheme:

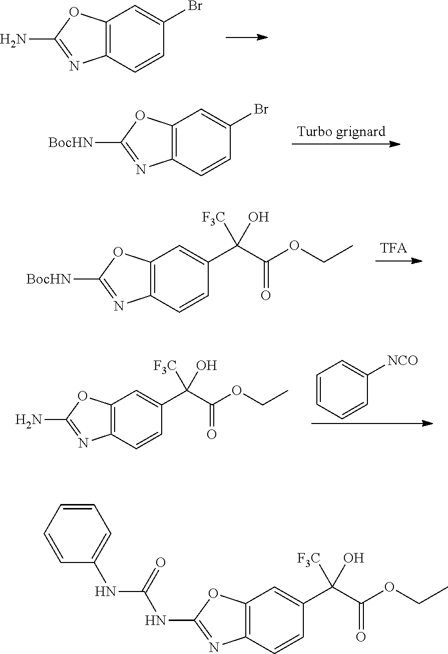

### Tert-Butyl (6-Bromobenzo[d]Oxazol-2-Yl) Carbamate

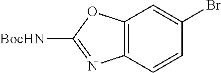 [SMILES: CC(C)(C)OC(=O)Nc1nc2ccc(Br)cc2o1]

Boc anhydride (246 mg, 1.13 mmol) and DMAP (5.78 mg, 0.047 mmol) were added to a solution of 6-bromo-1,3-benzoxazol-2-amine (0.2 g, 0.94 mmol) in DCM (4.7 mL). The resulting suspension was allowed to stir at 35° C. for two days. Upon completion, the reaction solvent was removed under reduced pressure. The resulting residue was purified by silica gel column chromatography (0-30% EtOAc/hexanes) to provide the title product (246 mg, 83.7%) as a brown solid. MS (ESI): mass calcd, for C₁₂H₁₃BrN₂O₃: 312.01, found: 312.80 [M+H]⁺.

### Ethyl 3,3,3-trifluoro-2-hydroxy-2-(2-(3-phenylureido)benzo[d]oxazol-6-yl)propanoate

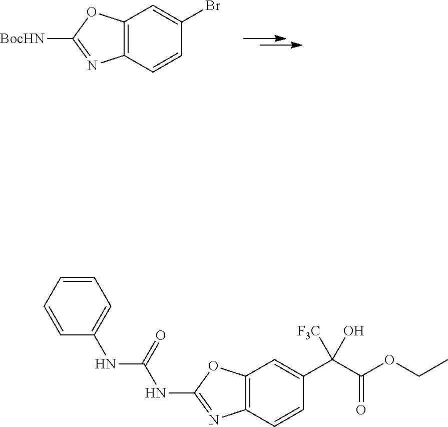 [SMILES: CC(C)(C)OC(=O)Nc1nc2ccc(Br)cc2o1 ; CCOC(=O)C(O)(c1ccc2nc(NC(=O)Nc3ccccc3)oc2c1)C(F)(F)F]

The title compound (60) (racemic) was synthesized from ethyl 3,3,3-trifluoro-2-hydroxy-2-(2-(3-phenylureido)quinolin-6-yl)propanoate in similar procedures as described in Example 8. MS (ESI): mass calcd, for C₁₀H₁₆F₄N₃O₅: 423.10, found: 423.95 [M+H]+. 1H NMR (400 MHz, CDCl₃) δ ppm 7.91 (s, 1H), 7.79 (d, J=8.5 Hz, 1H), 7.55-7.62 (m, 3H), 7.37 (t, J=7.8 Hz, 2H), 7.15 (t, J=7.4 Hz, 1H), 4.37-4.52 (m, 2H), 1.39 (t, J=7.1 Hz, 3H).

### Example 61. N-Ethyl-2-(2-(3-(4-fluorophenyl)ureido)benzo[d]thiazol-6-yl)-2-hydroxybutanamide (61)

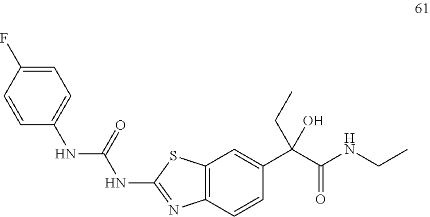 [SMILES: CCNC(=O)C(O)(CC)c1ccc2nc(NC(=O)Nc3ccc(F)cc3)sc2c1]

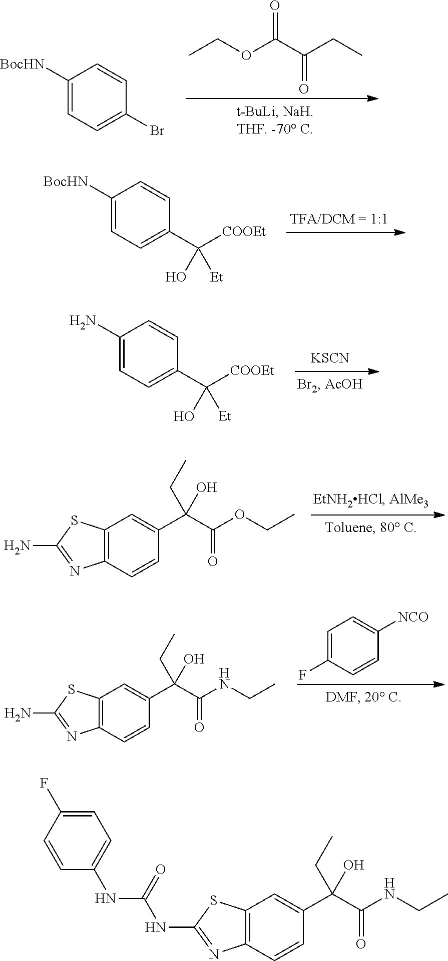

### Ethyl 2-[4-(tert-butoxycarbonylamino)phenyl]-2-hydroxy-butanoate

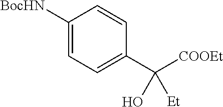 [SMILES: CCOC(=O)C(O)(CC)c1ccc(NC(=O)OC(C)(C)C)cc1]

To an ice cold solution of tert-butyl N-(4-bromophenyl)carbamate (3 g, 11.0 mmol) in THF (30 mL) was added NaH (441 mg, 11.0 mmol) under N₂. After stirring at 0° C. for 30 min, a solution of 1.3 M/-BuLi in heptane (9.3 mL, 10.3 mmol) was added dropwise at −70° C. The resulting solution was stirred at −70° C. for an additional 30 min, and ethyl 2-oxobutanoate (2.87 g, 22.1 mmol) was added. After 1 h at the same temperature, the reaction mixture was quenched with sat. NH₄Cl (30 mL) and extracted with ethyl acetate (30 mL×3). The combined organic layer was washed with brine (30 mL×2), dried over anhydrous Na₂SO₄, filtered, concentrated and purified by silica gel column chromatography (20-50% EtOAc/PE) to afford the title product (1.1 g, 30.9%) as a yellow solid. MS (EST): mass calcd. for C₁₇H₂₅NO₅: 323.17, found: 324.4 [M+H]⁺.

### Ethyl 2-(4-aminophenyl)-2-hydroxy-butanoate

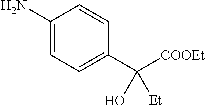 [SMILES: CCOC(=O)C(O)(CC)c1ccc(N)cc1]

A solution of ethyl 2-[4-(tert-butoxycarbonylamino)phenyl]-2-hydroxy-butanoate (1.1 g, 3.4 mmol) in TFA (5 mL) and DCM (5 mL) was stirred at 25° C. for 1 h. After completion, the reaction solvent was removed under reduced pressure. The resulting residue was diluted with DCM (10 mL) and washed with sat. NaHCO₃ (3 mL×2). The organic layer was dried over anhydrous Na₂SO₄, filtered and concentrated to afford the crude title product (631 mg, 83.1%) as a yellow oil, which was used in the next step without further purification. MS (ESI): mass calcd, for C₁₂H₁₇NO₃: 223.12, found: 224.1 [M+H]⁺.

### Ethyl 2-(2-amino-1,3-benzothiazol-6-yl)-2-hydroxy-butanoate

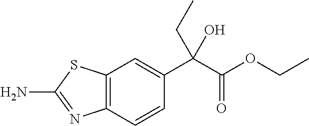 [SMILES: CCOC(=O)C(O)(CC)c1ccc2nc(N)sc2c1]

To a solution of ethyl 2-(4-aminophenyl)-2-hydroxy-butanoate (380 mg, 1.7 mmol) in AcOH (10 mL) was added KSCN (579 mg, 5.96 mmol). After stirring at 25° C. for 1 h, a solution of Br₂ (96.5 μL, 1.87 mmol) in AcOH (10 mL) was added. The reaction mixture was stirred at 25° C. for 12 h. After completion, the solution was filtered, and the filter cake was washed with EtOAc (10 mL×3). The filtrate was concentrated under reduced pressure. The resulting residue was treated with sat. NaHCO₃ (20 mL) to neutralize the remaining AcOH. The aqueous solution was extracted with ethyl acetate (20 mL×2). The combined organic layer was washed with brine (5 mL), dried over anhydrous Na₂SO₄, filtered, concentrated, and purified by silica gel column chromatography (6-50% EtOAc/PE) to obtain the title product (346 mg, 72.5%) as a yellow oil. MS (ESI): mass calcd, for C₁₃H₁₆N₂O₃S: 280.09, found: 281.1 [M+H]⁺.

### 2-(2-Amino-1,3-benzothiazol-6-yl)-N-ethyl-2-hydroxy-butanamide

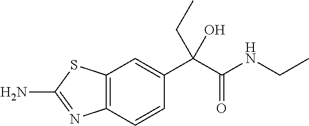 [SMILES: CCNC(=O)C(O)(CC)c1ccc2nc(N)sc2c1]

To a flask with ice-cold toluene (3 mL) was added a solution of 2 M AlMe₃ in toluene (6.06 mL, 12.1 mmol) and ethylamine hydrochloride (494.5 mg, 6.1 mmol). After stirring at this temperature for 10 min, ethyl 2-(2-amino-1,3-benzothiazol-6-yl)-2-hydroxy-butanoate (340 mg, 1.2 mmol) was added at 25° C. The reaction mixture was then heated at 80° C. under N2. After 2 h. the reaction was cooled to rt and quenched with sat. NH₄Cl (3 mL). The aqueous layer was extracted with EtOAc (3 mL×2). The combined organic layer was washed with brine, dried over Na₂SO₄, filtered, concentrated, and purified by silica gel column chromatography (50-100% EtOAc/PE) to afford the title product (180 mg, 53.1%) as a yellow oil. MS (ESI): mass calcd, for C₁₃H₁₇N₃O₂S: 279.10, found: 280.1 [M+H]⁺.

### N-Ethyl-2-(2-(3-(4-fluorophenyl)ureido)benzo[d]thiazol-6-yl)-2-hydroxybutanamide

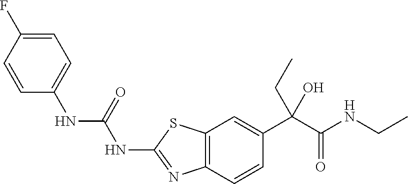 [SMILES: CCNC(=O)C(O)(CC)c1ccc2nc(NC(=O)Nc3ccc(F)cc3)sc2c1]

To a solution of 2-(2-amino-1,3-benzothiazol-6-yl)-N-ethyl-2-hydroxy-butanamide (180 mg, 0.64 mmol) in DMF (3 mL) was added 4-fluorophenyl isocyanate (72.2 μL, 0.64 mmol). After stirring at rt for 2 h. the reaction was quenched with sat. NH₄Cl (3 mL). The aqueous layer was extracted with EtOAc (10 mL×2). The combined organic layer was washed with brine, dried over anhydrous Na₂SO₄, filtered, concentrated and purified by reverse phase HPLC (gradient elution, 25-45% ACN in H₂O, with 0.05% NH₄HCO₃ as a modifier) to afford the title product (61) (153 mg, 57%, racemic) as a white solid. MS (ESI): mass calcd, for C₂₀H₂₁FN₄O₃S: 416.13, found: 417.1 [M+H]⁺. ¹H NMR (400 MHz, DMSO-d6) δ ppm 10.79 (br s, 1H), 9.21 (s, 1H), 8.01 (s, 1H), 7.85 (t, J=6.0 Hz, 1H), 7.49-7.61 (m, 4H), 7.17 (t, J=8.8 Hz, 2H), 5.84 (s, 1H), 2.97-3.14 (m, 2H), 2.17-2.26 (m, 1H), 1.85-1.96 (m, 1H), 0.97 (t, J=7.2 Hz, 3H), 0.80 (t, J=7.2 Hz, 3H).

### Example 67. N-Ethyl-3-(2-(3-(4-fluorophenyl)ureido)benzo[d]thiazol-6-yl)tetrahydrofuran-3-carboxamide (67)

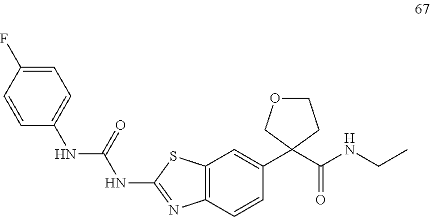 [SMILES: CCNC(=O)C1(c2ccc3nc(NC(=O)Nc4ccc(F)cc4)sc3c2)CCOC1]

### Synthetic Scheme:

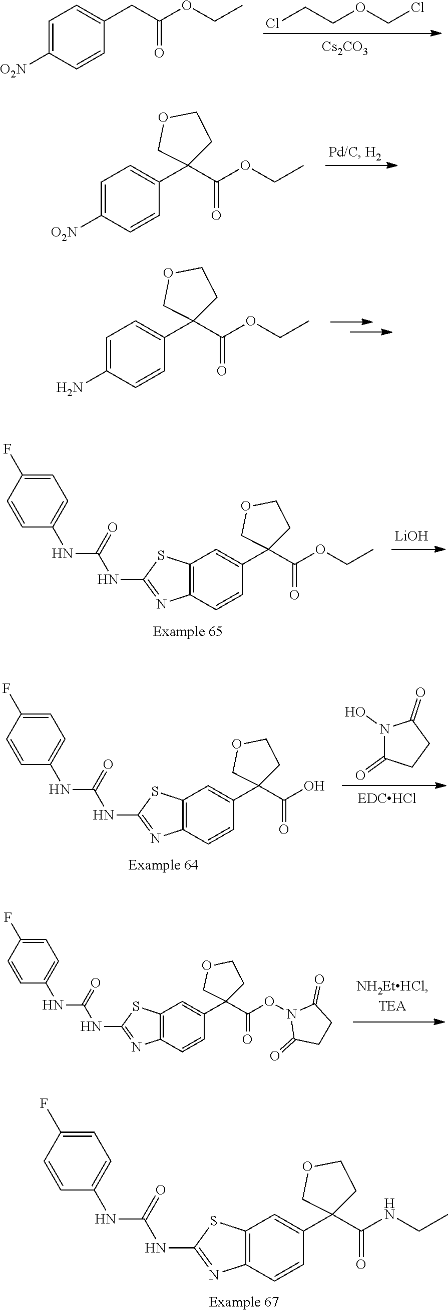

### Ethyl 3-(4-nitrophenyl)tetrahydrofuran-3-carboxylate

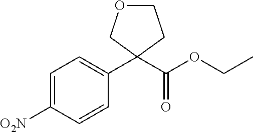 [SMILES: CCOC(=O)C1(c2ccc([N+](=O)[O-])cc2)CCOC1]

A suspension of ethyl 2-(4-nitrophenyl)acetate (1 g, 4.78 mmol), and Cs₂CO₃ (3.89 g, 12 mmol) in DMF (40 mL) was stirred at −40° C. for 15 min, followed by the addition of 1-chloro-2-(chloromethoxy) ethane (515 μL, 5.26 mmol). After stirring overnight at rt, the reaction was diluted with H₂O and EtOAc. The organic layer was washed with H₂O (×2) and brine (×3), dried over anhydrous Na₂SO₄, filtered, concentrated, and purified by silica gel column chromatography (0-30% EtOAc/hexanes) to provide the title product (0.2 g, 15.8%) as a yellow oil.

### Ethyl 3-(4-aminophenyl)tetrahydrofuran-3-carboxylate

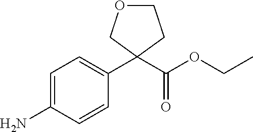 [SMILES: CCOC(=O)C1(c2ccc(N)cc2)CCOC1]

A suspension of ethyl 3-(4-nitrophenyl)tetrahydrofuran-3-carboxylate (0.2 g, 0.75 mmol) and 10% Pd/C (40.1 mg, 0.038 mmol) in EtOH (3.8 mL) was purged with H₂ three times. It was then allowed to stir under a H₂ balloon overnight. Upon completion, the reaction mixture was filtered through a syringe filter. The filtrate was concentrated to afford the crude title product (173 mg) as a yellow oil, which was used in the next step without further purification. MS (ESI): mass calcd, for C₁₃H₁₇NO₃: 235.12, found: 236.00 [M+H]⁺.

### Ethyl 3-(2-(3-(4-fluorophenyl)ureido)benzo[d]thiazol-6-yl)tetrahydrofuran-3-carboxylate (Example 65)

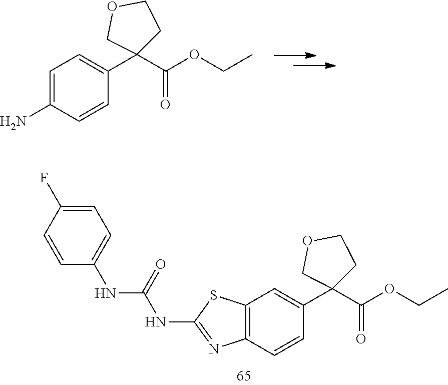 [SMILES: CCOC(=O)C1(c2ccc(N)cc2)CCOC1 ; CCOC(=O)C1(c2ccc3nc(NC(=O)Nc4ccc(F)cc4)sc3c2)CCOC1]

The title compound (65) (racemic) was synthesized from ethyl 3-(4-aminophenyl)tetrahydrofuran-3-carboxylate in similar procedures as described in Example 1, steps 3 and 4. MS (ESI): mass calcd, for C₂₁H₂₀FN₃O₄S: 429.12, found: 430.00 [M+H]⁺. ¹H NMR (400 MHz, CDCl₃) δ ppm 7.70 (d, J=1.7 Hz, 1H), 7.68 (d, J=8.6 Hz, 1H), 7.41-7.48 (m, 3H), 7.03 (t, J=8.6 Hz, 2H), 4.67 (d, J=8.6 Hz, 1H), 4.14 (q, J=7.1 Hz, 2H), 3.96-4.02 (m, 3H), 3.05 (dt, J=12.3, 6.1 Hz, 1H), 2.26 (dt, J=12.5, 8.4 Hz, 1H), 1.18 (t, J=7.1 Hz. 3H).

### 3-(2-(3-(4-Fluorophenyl)ureido)benzo[d]thiazol-6-yl)tetrahydrofuran-3-carboxylic acid (Example 64)

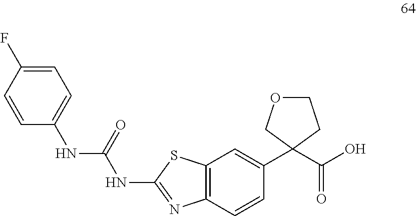 [SMILES: O=C(Nc1ccc(F)cc1)Nc1nc2ccc(C3(C(=O)O)CCOC3)cc2s1]

To a stirred solution of ethyl 3-(4-aminophenyl)tetrahydrofuran-3-carboxylate (85 mg, 0.20 mmol) in THE/MeOH/H₂O (4:1:1, 1 mL) was added lithium hydroxide monohydrate (23.7 mg, 0.99 mmol) at rt. After stirring overnight, AcOH was added to the mixture to adjust to pH=5. The resulting solution was partitioned between EtOAc and H₂O. The aqueous layer was extracted with 30% IPA/chloroform (×3). The combined organic layer was washed with brine, dried over anhydrous Na₂SO₄, filtered, concentrated, and purified by reverse phase HPLC (30-70% ACN/H₂O, with 0.1% TFA as a modifier) to afford the title product (64) (64 mg, 80.6%) as a white solid. MS (ESI): mass calcd. for C₁₉H₁₆FN₃O₄S: 401.08, found: 401.90 [M+H]⁺. ¹H NMR (400 MHz, CDCl₃) δ ppm 7.76 (s, 1H), 7.71 (d, J=8.5 Hz, 1H), 7.45-7.54 (m, 3H), 7.06 (t, J=8.4 Hz, 2H), 4.71 (d, J=8.6 Hz, 1H), 3.95-4.10 (m, 3H), 3.00-3.13 (m, 1H), 2.23-2.39 (m, 1H).

### 2,5-Dioxopyrrolidin-1-yl-3-(2-(3-(4-fluorophenyl)ureido)benzo[d]thiazol-6-yl)tetrahydrofuran-3-carboxylate

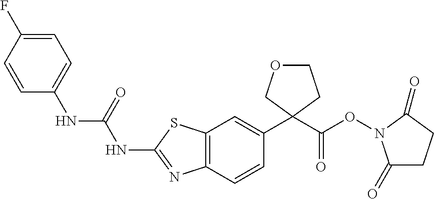 [SMILES: O=C(Nc1ccc(F)cc1)Nc1nc2ccc(C3(C(=O)ON4C(=O)CCC4=O)CCOC3)cc2s1]

A solution of 3-(2-(3-(4-fluorophenyl)ureido)benzo[d]thiazol-6-yl)tetrahydrofuran-3-carboxylic acid (53 mg, 0.12 mmol). N-hydroxysuccinimide (21.3 mg, 0.19 mmol), EDC (35.5 mg, 0.19 mmol) and DMAP (1.51 mg, 0.012 mmol) in DMF (1.2 mL) was stirred at rt. After 4 h, the reaction mixture was diluted with EtOAc. The organic layer was washed with brine (×3), dried over anhydrous Na₂SO₄, filtered and concentrated to afford the crude product (65 mg) as a yellow oil, which was directly used in the next step without further purification. MS (ESI): mass calcd, for C₂₃H₁₉FN₄O₆S: 498.10, found: 499.00 [M+H]⁺.

### N-Ethyl-3-(2-(3-(4-fluorophenyl)ureido)benzo[d]thiazol-6-yl)tetrahydrofuran-3-carboxamide

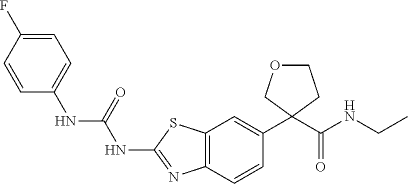 [SMILES: CCNC(=O)C1(c2ccc3nc(NC(=O)Nc4ccc(F)cc4)sc3c2)CCOC1]

Ethylamine hydrochloride (7.9 mg, 96.3 μmol) and TEA (20.1 μL, 144 μmol) were added to a solution of 2,5-dioxopyrrolidin-1-yl-3-(2-(3-(4-fluorophenyl)ureido)benzo[d]thiazol-6-yl)tetrahydrofuran-3-carboxylate (24 mg, 48.1 μmol) in THF (0.5 mL). The resulting solution was stirred at rt. Upon completion, the reaction solvent was removed under reduced pressure. The resulting residue was purified by reverse phase HPLC (35-65% ACN/H₂O, with 0.1% TFA as a modifier) to provide the title product (67) (13.9 mg, 67.3%, racemic) as a white solid. MS (ESI): mass calcd, for C₂₁H₂₁FN₄O₃S: 428.13, found: 429.00 [M+H]⁺. ¹H NMR (400 MHz, CDCl₃) δ ppm 7.72 (d, J=1.6 Hz, 1H), 7.69 (d, J=8.5 Hz, 1H), 7.47-7.50 (m, 2H), 7.42 (dd, J=8.5, 1.7 Hz, 1H), 7.06 (t, J=8.6 Hz, 2H), 5.66-5.72 (m, 1H), 4.43 (d, J=9.0 Hz, 1H), 4.08 (dd, J=15.2, 8.6 Hz, 2H), 3.98 (td, J=8.5, 5.7 Hz, 1H), 3.21-3.30 (m, 2H), 2.84-2.91 (m, 1H), 2.34 (ddd, J=12.6, 8.4, 6.8 Hz, 1H), 1.07 (t, J=7.2 Hz, 3H).

Example 62 was synthesized from methyl 1-(4-aminophenyl)cyclopentane-1-carboxylate in similar procedures as described in Example 65.

Example 63 was synthesized in similar procedures as described in Example 64. Examples 66, 68 and 69 were synthesized in similar procedures as described in Example 67.

### Example 72. 1-(4-Fluorophenyl)-3-(6-(1-(1-methyl-2,4-dioxo-1,4-dihydroquinazolin-3(2H)-yl)cyclopropyl)benzo[d]thiazol-2-yl)urea (72)

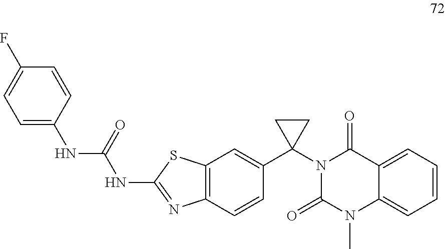 [SMILES: Cn1c(=O)n(C2(c3ccc4nc(NC(=O)Nc5ccc(F)cc5)sc4c3)CC2)c(=O)c2ccccc21]

### Synthetic Scheme:

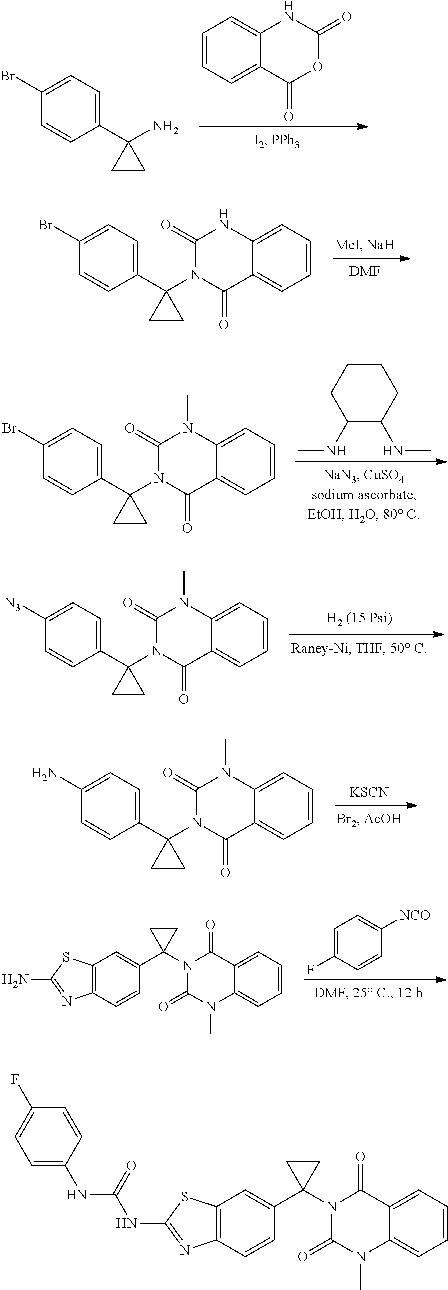

### 3-(1-(4-Bromophenyl)cyclopropyl)quinazoline-2,4(1H,3H)-dione

A solution of 12 (4.81 g, 18.9 mmol) and PPh₃ (4.97 g, 18.9 mmol) in DCM (30 mL) was stirred at 25° C. for 15 min. 2H-benzo[d][1,3]oxazine-2,4(1H)-dione (2.06 g, 12.6 mmol), 1-(4-bromophenyl)cyclopropanamine (3 g, 14.2 mmol) and TEA (7.03 mL, 50.5 mmol) in toluene (30 mL) were added at 0° C. After stirring at 25° C. for 16 h, the reaction was poured into water (40 mL) and the aqueous phase was extracted with EtOAc (40 mL×3). The combined organic layer was washed with brine, dried with anhydrous Na₂SO₄, filtered and concentrated under reduced pressure. The residue was purified by silica gel column chromatography (PE:EtOAc=1:0 to 2:3). The resulting residue was re-purified by reversed phase HPLC (30-65% ACN in H₂O (10 mM NH₄HCO₃)) to provide the title product (0.42 g, 9.1%) as a white powder. MS (ESI): mass calcd, for C₁₇H₁₃BrN₂O₂: 356.02, found: 357.1 [M+H]⁺.

### 3-(1-(4-Bromophenyl)cyclopropyl)-1-methylquinazoline-2,4(1H,3H)-dione

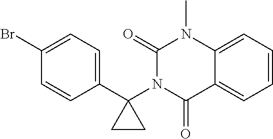 [SMILES: Cn1c(=O)n(C2(c3ccc(Br)cc3)CC2)c(=O)c2ccccc21]

To an ice cold solution of 3-[1-(4-bromophenyl)cyclopropyl]-1H-quinazoline-2,4-dione (0.25 g, 0.70 mmol) in DMF (5 mL) was added 60% NaH (30.8 mg, 0.77 mmol) in one portion. After stirring at rt for 30 min, MeI (47.9 μL, 0.77 mmol) was added. After 1.5 h, the reaction mixture was poured into sat. NH₄Cl (5 mL). The aqueous layer was extracted with EtOAc (5 mL×3). The combined organic layer was washed with brine (5 mL×2), dried over anhydrous Na₂SO₄, filtered, concentrated, and purified by prep-TLC (25% EtOAc/PE) to afford the title product (0.24 g, 91.8%) as a yellow powder. MS (ESI): mass calcd. for C₁₈H₁₅BrN₂O₂: 370.03, found: 371.1 [M+H]⁺.

### 3-(1-(4-Azidophenyl)cyclopropyl)-1-methylquinazoline-2,4(1H,3H)-dione

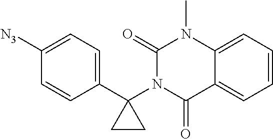 [SMILES: Cn1c(=O)n(C2(c3ccc(N=[N+]=[N-])cc3)CC2)c(=O)c2ccccc21]

To a solution of 3-(1-(4-bromophenyl)cyclopropyl)-1-methylquinazoline-2,4(1H,3H)-dione (0.24 g, 0.65 mmol) and NaN₃ (126.1 mg, 1.94 mmol) in EtOH (2 mL) and H₂O (0.4 mL) were added CuSO₄ (103.2 mg, 0.65 mmol), N1,N2-dimethylcyclohexane-1,2-diamine (55.18 mg, 0.39 mmol) and sodium ascorbate (256.2 mg, 1.29 mmol) in one portion at rt. After heating at 80° C. for 12 h, the reaction mixture was diluted with water (5 mL). The aqueous layer was extracted with MTBE (5 mL×3). The combined organic layer was washed with brine (5 mL), dried over anhydrous Na₂SO₄, filtered, concentrated to afford the crude title product (0.2 g), which was directly used in the next step without further purification. MS (ESI): mass calcd, for C₁₈H₁₅N₅O₂: 333.12, found: 334.3 [M+H]⁺.

### 3-(1-(4-Aminophenyl)cyclopropyl)-1-methylquinazoline-2,4(1H,3H)-dione

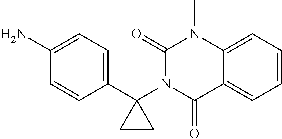 [SMILES: Cn1c(=O)n(C2(c3ccc(N)cc3)CC2)c(=O)c2ccccc21]

A solution of 3-(1-(4-azidophenyl)cyclopropyl)-1-methylquinazoline-2,4(1H,3H)-dione (0.2 g, 0.60 mmol) in THF (2 mL) was added Raney-Ni (0.1 g, 1.17 mmol) in one portion at 25° C. The resulting suspension was purged with H₂ (×3). It was then allowed to stir under H₂ (15 psi) at 50° C. for 1 h. The reaction mixture was filtered through a pad of celite and washed with EtOAc (2 mL×5). The filtrate was concentrated under reduced pressure and purified by prep-TLC (50% EtOAc/PE) to afford the title product (122 mg, 62.4%) as a yellow oil. MS (ESI): mass calcd, for C₁₈H₁₇N₃O₂: 307.13, found: 308.3 [M+H]⁺.

### 3-(1-(2-Aminobenzo[d]thiazol-6-yl)cyclopropyl)-1-methylquinazoline-2,4(1H,3H)-dione

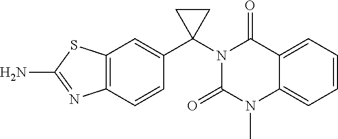 [SMILES: Cn1c(=O)n(C2(c3ccc4nc(N)sc4c3)CC2)c(=O)c2ccccc21]

To a solution of 3-(1-(4-aminophenyl)cyclopropyl)-1-methylquinazoline-2,4(1H,3H)-dione (45 mg, 0.15 mmol) in AcOH (1 mL) was added KSCN (49.8 mg, 0.51 mmol). After stirring at 25° C. for 1 h, a solution of Br₂ (8.3 μL, 0.16 mmol) in AcOH (1 mL) was added. The reaction mixture was stirred at 25° C. for another 3 h. After completion, the solution was filtered, and the filter cake was washed with EtOAc (10 ml×3). The filtrate was concentrated under reduced pressure. The resulting residue was treated with sat. NaHCO₃ (20 mL) to neutralize the remaining AcOH. The aqueous solution was extracted with ethyl acetate (20 mL×2). The combined organic layer was washed with brine (5 mL), dried over anhydrous Na₂SO₄, filtered, concentrated, and purified by prep-TLC (100% EtOAc) to afford the title product (17 mg, 31.9%) as a yellow solid. MS (ESI): mass calcd, for C₁₉H₁₆N₄O₂S: 364.10, found: 365.2 [M+H]⁺.

### 1-(6-(1-(2,4-Dioxo-1,4-dihydroquinazolin-3 (2H)-yl)cyclopropyl)benzo[d]thiazol-2-yl)-3-(4-fluorophenyl)urea

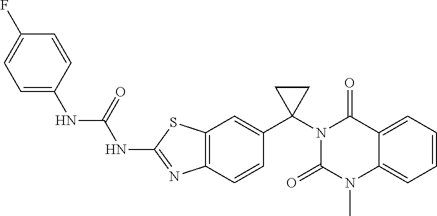 [SMILES: Cn1c(=O)n(C2(c3ccc4nc(NC(=O)Nc5ccc(F)cc5)sc4c3)CC2)c(=O)c2ccccc21]

To a solution of 3-(1-(2-aminobenzo[d]thiazol-6-yl)cyclopropyl)-1-methylquinazoline-2,4(1H,3H)-dione (17 mg, 0.047 mmol) in DMF (1 mL) was added 1-fluoro-4-isocyanato-benzene (6.27 μL, 0.056 mmol). After stirring at rt for 4 h, the reaction was quenched by sat. NH₄Cl (3 mL). The aqueous layer was extracted with EtOAc (10 mL×2). The combined organic layer was washed with brine, dried over anhydrous Na₂SO₄, filtered, concentrated and purified by reverse phase HPLC (gradient elution, 30-65% ACN in H₂O, with 0.05% NH₄HCO₃ as a modifier) to afford the title product (72) (6.4 mg, 26.9%) as a white solid. MS (ESI): mass calcd, for C₂₆H₂₀FN₅O₃S: 501.13, found: 502.1 [M+H]⁺. ¹H NMR (400 MHz, DMSO-d₆) δ ppm 10.68 (br s, 1H), 9.12-9.29 (m, 1H), 8.04-8.09 (m, 1H), 7.81-7.86 (m, 1H), 7.75-7.81 (m, 1H), 7.51-7.56 (m, 2H), 7.45 (d, J=8.4 Hz, 1H), 7.27-7.34 (m, 2H), 7.15 (t, J=8.8 Hz, 2H), 3.52 (s, 3H), 1.56-1.61 (m, 2H), 1.36-145 (m, 2H).

Example 73 was synthesized in similar procedures as described in Example 72, without the methylation step.

### Example 74. N-Ethyl-2-(2-(3-(4-fluorophenyl)ureido)benzo[d]thiazol-6-yl)pyrrolidine-2-carboxamide (74)

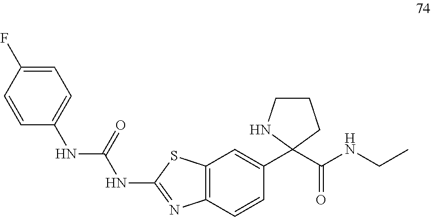 [SMILES: CCNC(=O)C1(c2ccc3nc(NC(=O)Nc4ccc(F)cc4)sc3c2)CCCN1]

### Synthetic Scheme:

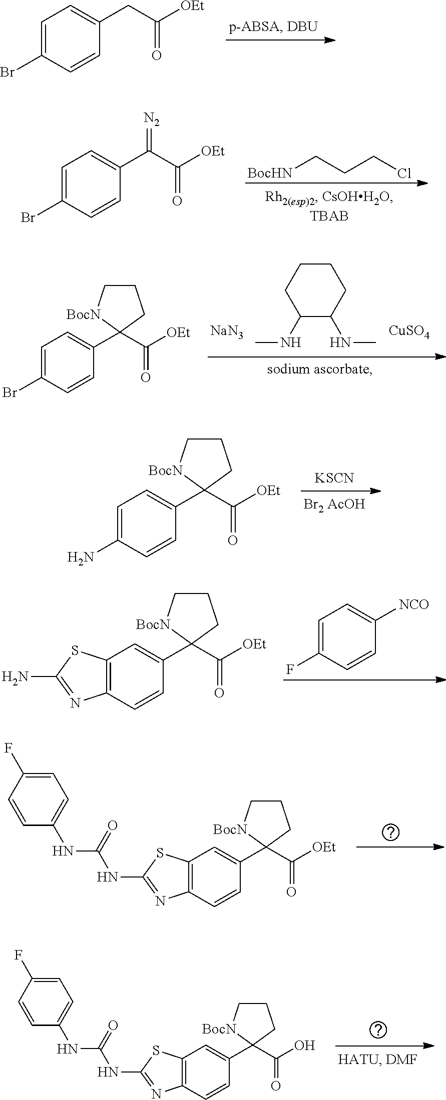

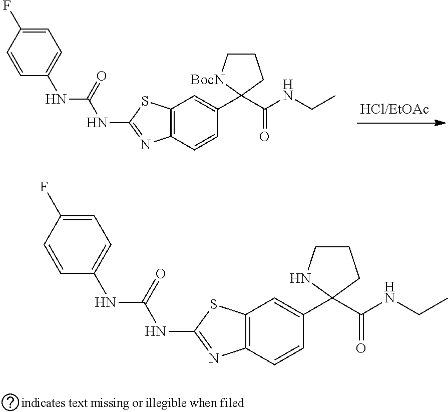 [SMILES: CCNC(=O)C1(c2ccc3nc(NC(=O)Nc4ccc(F)cc4)sc3c2)CCCN1C(=O)OC(C)(C)C ; CCNC(=O)C1(c2ccc3nc(NC(=O)Nc4ccc(F)cc4)sc3c2)CCCN1]

### Ethyl 2-(4-bromophenyl)-2-diazoacetate

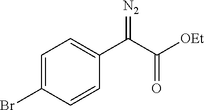

To an ice cold solution of ethyl 2-(4-bromophenyl)acetate (5 g, 20.6 mmol) in ACN (70 mL) was added 2,3,4,6,7,8,9,10-octahydropyrimido[1,2-a]azepine (3.72 mL, 24.7 mmol) and N-(4-azidosulfonylphenyl) acetamide (4.94 g, 20.6 mmol). After stirring at rt overnight, the reaction was diluted with H₂O (300 mL). The aqueous layer was extracted with EtOAc (100 mL×3). The combined organic layer was washed with brine, dried over Na₂SO₄, filtered, concentrated, and purified by silica gel column chromatography (0-20% EtOAc/PE) to afford the title product. ¹H NMR (400 MHz, CDCl₃) δ ppm 7.50 (d, J=8.8 Hz, 2H), 7.38 (d, J=8.0 Hz, 2H), 4.31-4.36 (m, 2H), 1.34 (t, J=7.2 Hz, 3H).

### 1-(tert-Butyl) 2-ethyl 2-(4-bromophenyl)pyrrolidine-1,2-dicarboxylate

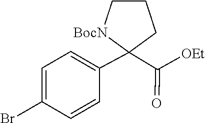 [SMILES: CCOC(=O)C1(c2ccc(Br)cc2)CCCN1C(=O)OC(C)(C)C]

To a solution of ethyl 2-(4-bromophenyl)-2-diazo-acetate (200 mg, 0.74 mmol) and tert-butyl N-(3-chloropropyl)carbamate (96 mg, 0.50 mmol) in toluene (50 mL) was added cesium hydroxide (148.6 mg, 0.99 mmol), Rh₂(esp)₂ (18.9 mg, 0.025 mmol) and TBAB (15.97 mg, 49.55 μmol). After heating at 60° C. overnight, the reaction was diluted with H₂O (15 mL). The aqueous layer was extracted with EtOAc (25 mL×3). The combined organic layer was washed with brine, dried over Na₂SO₄, filtered, concentrated, and purified by silica gel column chromatography (0-20% EtOAc/PE) to afford the title product (50 mg, 25.3%) as a white solid. MS (ESI): mass calcd, for C₁₈H₂₄BrNO₄: 397.09, found: 398.3 [M+H]⁻.

### 1-(tert-Butyl) 2-ethyl 2-(2-(3-(4-fluorophenyl)ureido)benzo[d]thiazol-6-yl)pyrrolidine-1,2-dicarboxylate

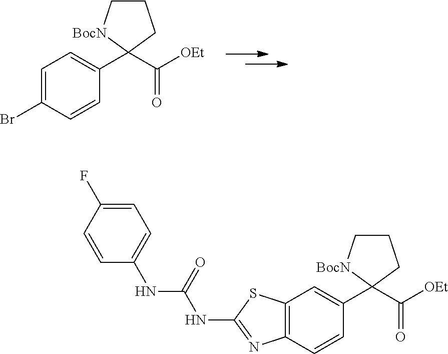 [SMILES: CCOC(=O)C1(c2ccc(Br)cc2)CCCN1C(=O)OC(C)(C)C ; CCOC(=O)C1(c2ccc3nc(NC(=O)Nc4ccc(F)cc4)sc3c2)CCCN1C(=O)OC(C)(C)C]

The title compound was synthesized from 1-(tert-butyl) 2-ethyl 2-(4-bromophenyl)pyrrolidine-1,2-dicarboxylate in similar procedures as described in Example 41. MS (ESI): mass calcd, for C₂₆H₂₉FN₄O₅S: 528.18, found: 529.3 [M+H]⁺.

### tert-Butyl 2-(ethylcarbamoyl)-2-(2-(3-(4-fluorophenyl)ureido)benzo[d]thiazol-6-yl)pyrrolidine-1-carboxylate

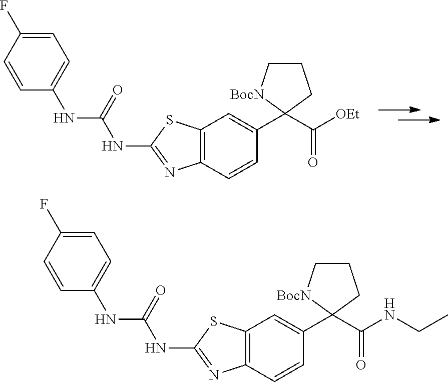 [SMILES: CCOC(=O)C1(c2ccc3nc(NC(=O)Nc4ccc(F)cc4)sc3c2)CCCN1C(=O)OC(C)(C)C ; CCNC(=O)C1(c2ccc3nc(NC(=O)Nc4ccc(F)cc4)sc3c2)CCCN1C(=O)OC(C)(C)C]

The title compound was synthesized from 1-(tert-butyl) 2-ethyl 2-(2-(3-(4-fluorophenyl)ureido)benzo[d]thiazol-6-yl)pyrrolidine-1,2-dicarboxylate in similar procedures as described in Example 38. MS (ESI): mass calcd, for C₂₆H₃₀FN₅O₄S: 527.20, found: 528.3 [M+H]⁺.

### N-Ethyl-2-(2-(3-(4-fluorophenyl)ureido)benzo[d]thiazol-6-yl)pyrrolidine-2-carboxamide

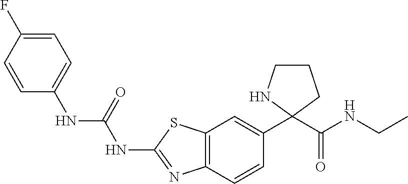 [SMILES: CCNC(=O)C1(c2ccc3nc(NC(=O)Nc4ccc(F)cc4)sc3c2)CCCN1]

A solution of tert-butyl 2-(ethylcarbamoyl)-2-(2-(3-(4-fluorophenyl)ureido)benzo[d]thiazol-6-yl)pyrrolidine-1-carboxylate (0.1 g, 0.19 mmol) in EtOAc (1 mL) and HCl/EtOAc (1 mL) was stirred at rt for 1 h. Upon completion, the reaction was quenched by NaHCO₃ to adjust to pH=8. The aqueous layer was extracted with EtOAc (3 mL×2). The combined organic layer was dried over Na₂SO₄, filtered, concentrated, and purified by reverse phase HPLC (20-50% ACN/H₂O, with 0.05% NH₄HCO₃) to afford the title product (74) (23 mg, 2.84%, racemic) as a white solid. MS (ESI): mass calcd. for C₂₁H₂₂FN₅O₂S: 427.15, found: 428.3 [M+H]⁺. ¹H NMR (400 MHz, DMSO-d₆) δ ppm 10.92 (br s, 1H), 9.24 (s, 1H), 8.24 (t, J=6.0 Hz, 1H), 7.91 (s, 1H), 7.49-7.58 (m, 3H), 7.41-7.44 (m, 1H), 7.16 (t, J=8.8 Hz, 2H), 2.96-3.12 (m, 3H), 2.66-2.84 (m, 2H), 1.72-1.85 (m, 2H), 1.54-1.66 (m, 1H), 0.96 (t, J=7.2 Hz, 3H).

Example 81 was synthesized in similar procedures as described in Example 74.

### Example 77, Ethyl 3,3,3-trifluoro-2-hydroxy-2-(2-((phenoxycarbonyl)amino)benzo[d]thiazol-6-yl)propanoate (77)

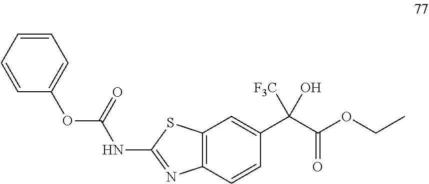 [SMILES: CCOC(=O)C(O)(c1ccc2nc(NC(=O)Oc3ccccc3)sc2c1)C(F)(F)F]

### Synthetic Scheme:

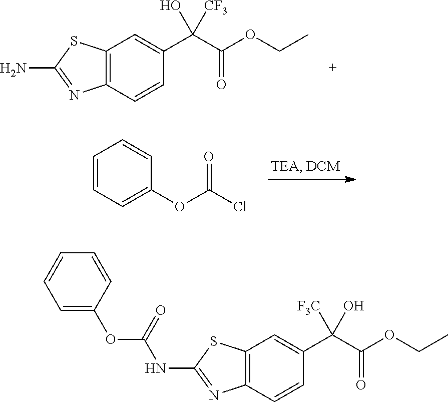

To a solution of ethyl 2-(2-amino-1,3-benzothiazol-6-yl)-3,3,3-trifluoro-2-hydroxy-propanoate (50 mg, 0.16 mmol) in DCM (2 mL) was added TEA (108.6 μL, 0.78 mmol) and phenyl carbonochloridate (58.7 μL, 0.47 mmol) at 0° C. After stirring at rt for 1 h, the reaction solvent was removed under reduced pressure. The resulting residue was partitioned between H₂O (2 mL) and DCM (2 mL). The aqueous layer was extracted with DCM (2 mL×2). The combined organic layer was filtered, concentrated and purified by reversed phase HPLC (40-70% ACN/H₂O, with 0.1% TEA as a modifier) to afford the title product (77) (6 mg, 8.5%, racemic) as a white solid. MS (ESI): mass calcd, for C₁₉H₁₅F₃N₂O₅S: 440.07, found: 441.10 [M+H]⁺. ¹H NMR (400 MHz, DMSO-d₆) δ ppm 12.70 (br s, 1H), 8.20 (d, J=1.2 Hz, 1H), 7.93 (s, 1H), 7.79 (d, J=8.8 Hz, 1H), 7.79 (d, J=8.8 Hz, 1H), 7.47 (t, J=7.6 Hz, 2H), 7.25-7.35 (m, 2H), 4.27-4.30 (m, 2H), 1.22 (t, J=7.2 Hz, 3H).

### Example 83. (S)-3,3,3-Trifluoro-2-(2-(3-(4-fluorophenyl)ureido)benzo[d]thiazol-6-yl)-2-hydroxy-N-(oxetan-3-yl)propenamide (83)

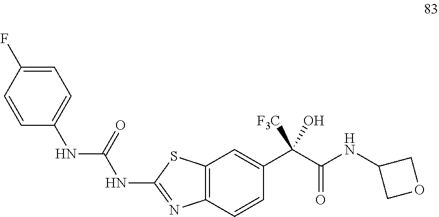 [SMILES: O=C(Nc1ccc(F)cc1)Nc1nc2ccc([C@](O)(C(=O)NC3COC3)C(F)(F)F)cc2s1]

### Synthetic Scheme:

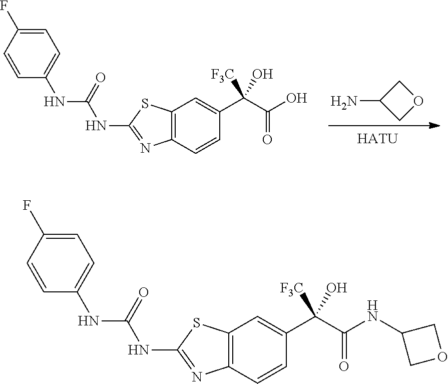

To a solution of (S)-3,3,3-trifluoro-2-(2-(3-(4-fluorophenyl)ureido)benzo[d]thiazol-6-yl)-2-hydroxypropanoic acid (described in Example 38, 0.03 g, 0.07 mmol) in DMF (1 mL) was added DIPEA (36.5 μL, 0.021 mmol) and HATU (53.1 mg, 0.14 mmol) at 0° C. Oxetan-3-amine (20.4 mg, 0.28 mmol) was then added. After stirring at 20° C. for 2 h, the reaction mixture was quenched with H₂O (2 mL) and extracted with EtOAc (2 mL×2). The combined organic layer was washed with brine, dried over Na₂SO₄, filtered and concentrated under reduced pressure. The resulting residue was purified by reverse phase HPLC (20-45% ACN in H₂O (10 mM NH₄HCO₃), gradient separation) to provide the title product (83) (18.2 mg, 16%) as a white solid. MS (ESI): mass calcd, for C₂₀H₁₆F₄N₄O₄S: 484.08, found: 485.0 [M+H]⁺. ¹H NMR (400 MHz, DMSO-d₆) δ ppm 10.86 (br s, 1H), 9.17-9.26 (m, 1H), 8.93 (d, J=6.4 Hz, 1H), 8.15 (s, 1H), 7.91 (s, 1H), 7.50-7.67 (m, 4H), 7.17 (t, J=8.8 Hz, 2H), 4.76-4.85 (m, 1H), 4.52-4.69 (m, 3H), 4.43-4.49 (m, 1H).

Examples 76, 78, 79, 80, and 82 were synthesized in similar procedures as described in Example 83.

Example 84, 85 and 86 were synthesized in similar procedures as described in Example 83, using their respective racemic carboxylic acid precursor.

Examples 87, 88, 89, and 90 were synthesized in similar procedures as described in Example 38.

### Example 91. (S)-2-(2-(3-((R)-1-Cyclobutylethyl)ureido)benzo[d]thiazol-6-yl)-N-ethyl-3,3,3-trifluoro-2-hydroxypropanamide (91)

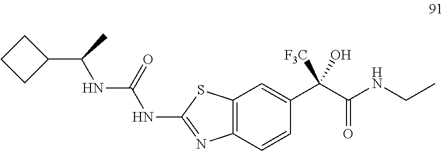 [SMILES: CCNC(=O)[C@@](O)(c1ccc2nc(NC(=O)N[C@H](C)C3CCC3)sc2c1)C(F)(F)F]

### Synthetic Scheme:

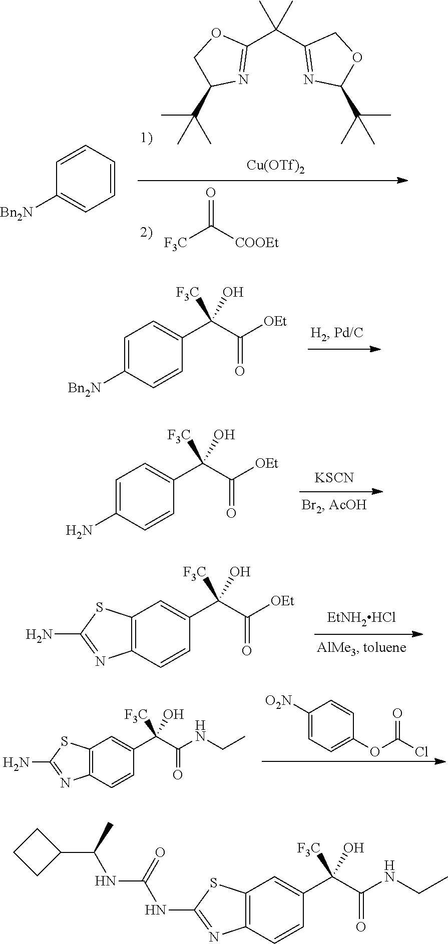

### Ethyl (S)-2-(4-(dibenzylamino)phenyl)-3,3,3-trifluoro-2-hydroxypropanoate

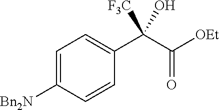

(4S)-4-tert-butyl-2-[1-[(4S)-4-tert-butyl-4,5-dihydrooxazol-2-yl]-1-methyl-ethyl]-4,5-dihydrooxazole (5.39 g, 18.29 mmol) was added to a suspension of Cu(OTf)₂ (6.62 g, 18.3 mmol) in MTBE (100 mL) at rt. After stirring for 15 min, a solution of ethyl 3,3,3-trifluoro-2-oxo-propanoate (36.4 mL, 274.4 mmol) and N,N-dibenzylaniline (50 g, 182.9 mmol) in MTBE (200 mL) was added. The resulting solution was further stirred for 18 b at rt. Upon completion, the reaction mixture was poured into water (300 mL) and extracted with EtOAc (300 mL×3). The combined organic layer was dried with anhydrous Na₂SO₄, filtered, concentrated and purified by silica gel column chromatography (0-100% EtOAc/PE) to afford the title compound (80 g, 98.6%, ee: 84.8%) as a white solid. MS (ESI): mass calcd. for C₂₅H₂₄F₃NO₃: 443.17, found: 444.2 [M+H]⁺.

### Ethyl (S)-2-(4-aminophenyl)-3,3,3-trifluoro-2-hydroxypropanoate

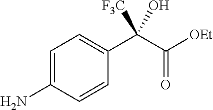 [SMILES: CCOC(=O)[C@@](O)(c1ccc(N)cc1)C(F)(F)F]

To a solution of ethyl (S)-2-(4-(dibenzylamino)phenyl)-3,3,3-trifluoro-2-hydroxypropanoate (20 g, 45.1 mmol) in MeOH (200 mL) was added 10% Pd/C (4.8 g, 4.5 mmol) under N₂. The resulting suspension was purged with H₂ (×3) and then stirred under H₂ (15 Psi) for 12 h. The reaction was filtered through a celite pad and the filter cake was washed with MeOH (800 mL). The filtrate was concentrated and purified by silica gel column chromatography (0-100% EtOAc/PE) to afford the title compound (46 g, 96.9%) as a white solid. MS (ESI): mass calcd, for C₁₁H₁₂F₃NO₃: 263.08, found: 264.2 [M+H]⁺.

### (S)-2-(2-Aminobenzo[d]thiazol-6-yl)-N-ethyl-3,3,3-trifluoro-2-hydroxypropanamide

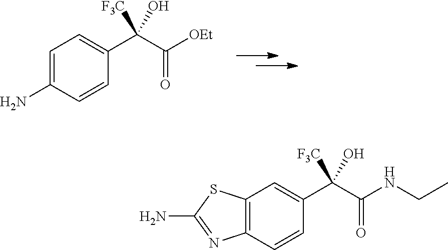 [SMILES: CCOC(=O)[C@@](O)(c1ccc(N)cc1)C(F)(F)F ; CCNC(=O)[C@@](O)(c1ccc2nc(N)sc2c1)C(F)(F)F]

The title compound was prepared from ethyl (S)-2-(4-aminophenyl)-3,3,3-trifluoro-2-hydroxypropanoate following Example 61, step 3 and 4 procedures, and further purified by chiral SFC separation (column: DAICEL CHIRALPAK AD (250 mm*50 mm, 10 μm); mobile phase: 25% EtOH with 0.1% NH₃H₂O) to afford the title product as a white solid (ee: 99.7%). MS (ESI): mass calcd, for C₁₂H₁₂F₃N₃O₂S: 319.06, found: 320.0 [M+H]⁺.

### (S)-2-(2-(3-((R)-1-Cyclobutylethyl)ureido)benzo[d]thiazol-6-yl)-N-ethyl-3,3,3-trifluoro-2-hydroxypropanamide (91)

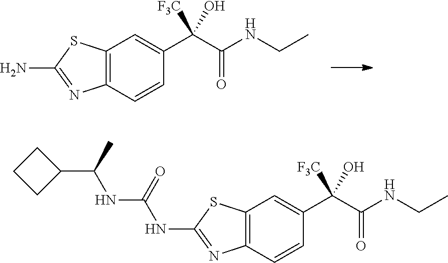 [SMILES: CCNC(=O)[C@@](O)(c1ccc2nc(N)sc2c1)C(F)(F)F ; CCNC(=O)[C@@](O)(c1ccc2nc(NC(=O)N[C@H](C)C3CCC3)sc2c1)C(F)(F)F]

The title compound was synthesized from (S)-2-(2-aminobenzo[d]thiazol-6-yl)-N-ethyl-3,3,3-trifluoro-2-hydroxypropanamide and (R)-1-cyclobutylethan-1-amine (HCl salt) in similar procedures as described in Example 2. MS (ESI): mass calcd, for C₁₉H₂₃F₃N₄O₃S: 444.14, found: 444.95 [M+H]⁺. ¹H NMR (400 MHz DMSO-d6): δ ppm 10.49 (s, 1H), 8.20 (t, J=5.9 Hz, 1H), 8.12 (s, 1H), 7.74 (s, 1H), 7.62 (s, 2H), 6.58 (d, J=8.3 Hz, 1H), 3.65-3.74 (m, 1H), 3.08-3.17 (m, 2H), 2.27-2.37 (m, 1H), 1.91-1.99 (m, 2H), 1.68-1.84 (m, 4H), 0.97-1.02 (m, 6H).

### Example 92. Ethyl 3,3,3-trifluoro-2-hydroxy-2-(1-methyl-2-(3-phenylureido)-1H-benzo[d]imidazol-5-yl)propanoate (92)

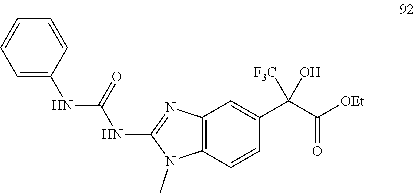 [SMILES: CCOC(=O)C(O)(c1ccc2c(c1)nc(NC(=O)Nc1ccccc1)n2C)C(F)(F)F]

### Synthetic Scheme:

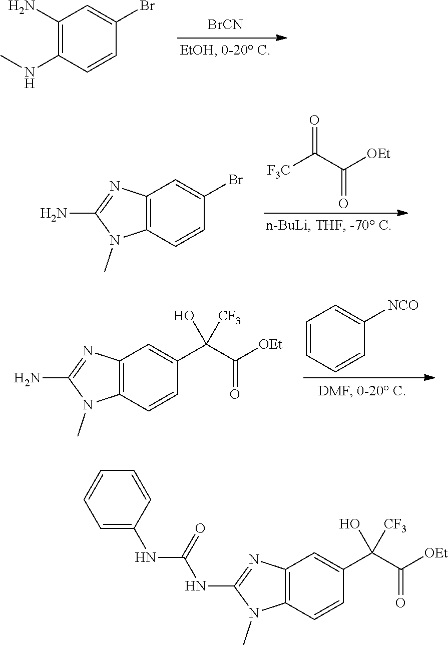

### 5-Bromo-1-methyl-benzimidazol-2-amine

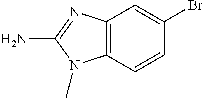

To a solution of 4-bromo-N1-methyl-benzene-1,2-diamine (5 g, 24.87 mmol) in EtOH (60 mL) was added BrCN (2.9 g, 27.35 mmol) at 0° C. After stirring at 20° C. for 12 h, the reaction was quenched with H₂O (60 mL) and extracted with DCM (60 mL×2). The combined organic layer was washed with brine, dried over Na₂SO₄, filtered and concentrated under reduced pressure to provide the title compound (5 g, 88.9% yield) as a yellow solid, which was used directly in the next step without further purification. MS (ESI): mass calcd. for C₈H₈BrN₃: 224.99, found: 226.0 [M+H]⁺.

### Ethyl 2-(2-amino-1-methyl-benzimidazol-S-yl)-3,3,3-trifluoro-2-hydroxy-propanoate

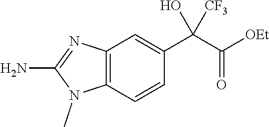 [SMILES: CCOC(=O)C(O)(c1ccc2c(c1)nc(N)n2C)C(F)(F)F]

To a solution of 5-bromo-1-methyl-benzimidazol-2-amine (3 g, 13.3 mmol) in THF (100 mL) was added NaH (1.17 g, 29.2 mmol, 60% in mineral oil) in portions under N₂ at 0° C. After stirring at 25° C. for 30 min, t-BuLi (1.3 M, 25.52 mL) was added dropwise at −78° C. and the reaction was stirred at −78° C. for 30 min. Ethyl 3,3,3-trifluoro-2-oxo-propanoate (2.71 g, 15.92 mmol) was added. After stirring at the same temperature for 1 h, the reaction was quenched with saturated NH₄Cl (100 mL) and extracted with EtOAc (100 mL×2). The combined organic layer was washed with brine, dried over Na₂SO₄, filtered and concentrated under reduced pressure. The crude residue was purified by reverse phase HPLC (column: Welch Xtimate C18 180*70 mm; 10 μm; mobile phase: [H₂O (10 mM NH₄HCO₃) ACN]; gradient elution: 20-50% B) to provide the title compound (500 mg, 11.9%) as a yellow oil. MS (ESI): mass calcd, for C₁₃H₁₄F₃N₃O₃: 317.10, found: 318.2 [M+H]⁺.

### Ethyl 3,3,3-trifluoro-2-hydroxy-2-(1-methyl-2-(3-phenylureido)-1H-benzo[d]imidazol-5-yl)propanoate (92)

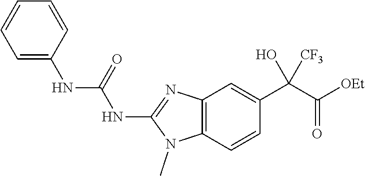 [SMILES: CCOC(=O)C(O)(c1ccc2c(c1)nc(NC(=O)Nc1ccccc1)n2C)C(F)(F)F]

To a mixture of ethyl 2-(2-amino-1-methyl-benzimidazol-5-yl)-3,3,3-trifluoro-2-hydroxy-propanoate (30 mg, 0.095 mmol) in DMF (1 mL) was added isocyanatobenzene (16.9 mg, 0.14 mmol) at 0° C. under N₂. After stirring at 25° C. for 2 h, the reaction was quenched with saturated NH₄Cl (10 mL) and extracted with EtOAc (5 mL×2). The combined organic layer was washed with brine, dried over Na₂SO₄, filtered and concentrated under reduced pressure. The crude residue was purified by reverse phase HPLC (mobile phase: [H₂O (10 mM NH₄HCO₃)-ACN]; gradient: 45-65% B) to provide the title compound (92) (4 mg, 9.7%, racemic) as a white solid. MS (ESI): mass calcd, for C₂₀H₁₉F₃N₃O₄: 436.14, found: 437.1 [M+H]⁺. ¹H NMR (400 MHz, DMSO-d₆) δ ppm 9.12-9.17 (m, 1H), 7.88 (s, 1H), 7.81 (s, 1H), 7.70 (d, J=7.6 Hz, 2H), 7.47 (d, J=2.8 Hz, 1H), 7.32-7.40 (m, 2H), 7.24 (d, J=6.8 Hz, 2H), 6.87-6.92 (m, 1H), 4.29 (q, J=6.4 Hz, 2H), 3.55 (s, 3H), 1.22-1.27 (m, 3H).

Examples 118 and 148 were synthesized in similar procedures as described in Example 92.

### Example 93. 1-(6-(2-Ethyl-1,3-dioxotetrahydro-1H-pyrrolo[1,2-c]imidazol-7a(5H)-yl)benzo[d]thiazol-2-yl)-3-(4-fluorophenyl)urea (93)

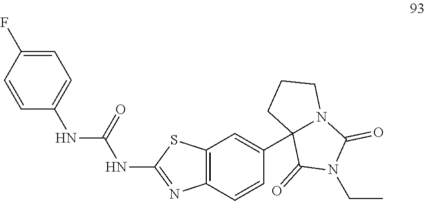 [SMILES: CCN1C(=O)N2CCCC2(c2ccc3nc(NC(=O)Nc4ccc(F)cc4)sc3c2)C1=O]

### Synthetic Scheme:

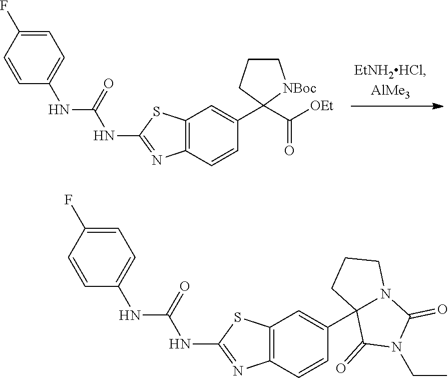 [SMILES: CCOC(=O)C1(c2ccc3nc(NC(=O)Nc4ccc(F)cc4)sc3c2)CCCN1C(=O)OC(C)(C)C ; CCN1C(=O)N2CCCC2(c2ccc3nc(NC(=O)Nc4ccc(F)cc4)sc3c2)C1=O]

### 1-(6-(2-Ethyl-1,3-dioxotetrahydro-1H-pyrrolo[1,2-c]imidazol-7a (5H)-yl)benzo[d]thiazol-2-yl)-3-(4-fluorophenyl)urea (93)

To an ice cold solution of ethanamine hydrochloride (61.89 μL, 0.95 mmol) in toluene (2 mL) was added 2 M AlMe₃ (236 μL, 0.47 mmol). After heating at 40° C. for 0.5 h, 1-(tert-butyl) 2-ethyl 2-(2-(3-phenylureido)benzo[d]thiazol-6-yl)pyrrolidine-1,2-dicarboxylate (see Example 74 for synthesis) (50 mg, 95 μmol) was added. The resulting solution was then heated at 80° C. for 12 h. The reaction was quenched with sat. NH₄Cl solution (5 mL) and extracted with EtOAc (5 mL×3). The combined organic layer was washed with brine, dried over Na₂SO₄, filtered, concentrated and purified by reverse phase HPLC (30-60% ACN in H₂O (0.2% FA), gradient elution) to afford the title compound (93) (2.1 mg, 4.9%, racemic) as a white solid. MS (ESI): mass calcd, for C₂₂H₂₀FN₅O₃S: 453.13, found: 454.1 [M+H]⁺. ¹H NMR (400 MHz, DMSO-d₆) δ ppm 11.17 (s, 1H), 9.39 (s, 1H), 8.04 (s, 1H), 7.64 (d, J=8.0 Hz, 1H), 7.50-7.58 (m, 3H), 7.16 (t, J=8.8 Hz, 2H), 3.58-3.76 (m, 2H), 3.38-3.41 (m, 2H), 2.38-2.50 (m, 1H), 2.06-2.18 (m, 2H), 1.69-1.80 (m, 1H), 1.05 (t, J=7.2 Hz, 3H).

### Example 94. 1-(4-Fluorophenyl)-3-(6-(2,2,2-trifluoro-1-hydroxy-1-(5-methyl-1H-imidazol-2-yl)ethyl)benzo[d]thiazol-2-yl)urea (94)

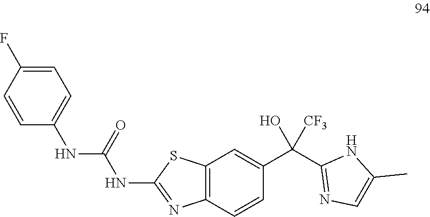 [SMILES: Cc1cnc(C(O)(c2ccc3nc(NC(=O)Nc4ccc(F)cc4)sc3c2)C(F)(F)F)[nH]1]

### Synthetic Scheme:

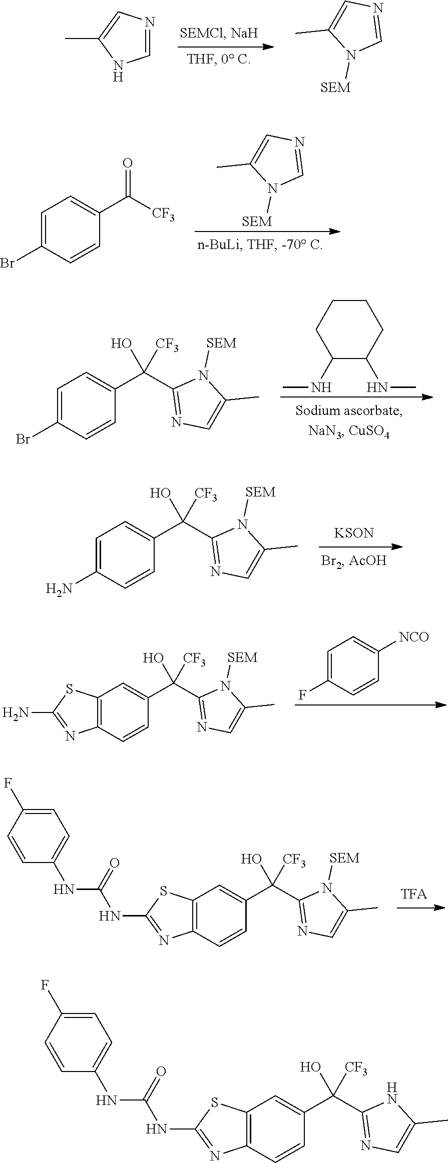

### 5-Methyl-1-((2-(trimethylsilyl)ethoxy)methyl)-1H-imidazole

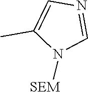

To a solution of 5-methyl-1H-imidazole (4.5 g, 54.81 mmol) in THF (50 mL) was added 60% NaH (4.38 g, 109.6 mmol) at −70° C. under N₂. After stirring at this temperature for 30 min, SEMCl (9.7 mL, 54.8 mmol) was added dropwise. The reaction was stirred for 2 h, quenched with sat. NH₄Cl (100 mL) and extracted with EtOAc (100 mL×3). The combined organic layer was washed with brine, dried over anhydrous Na₂SO₄, filtered, and concentrated under reduced pressure to afford the title product (8 g, crude) as a yellow oil, which was directly used in the next step without further purification.

### 1-(4-Bromophenyl)-2,2,2-trifluoro-1-(5-methyl-1-((2-(trimethylsilyl)ethoxy)methyl)-1H-imidazol-2-yl) ethan-1-ol

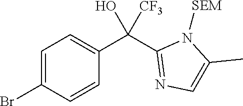 [SMILES: Cc1cnc(C(O)(c2ccc(Br)cc2)C(F)(F)F)n1COCC[Si](C)(C)C]

n-BuLi (1.88 mL, 4.7 mmol, 2.5 M) was added to a solution of trimethyl-[2-[(5-methylimidazol-1-yl)methoxy]ethyl]silane (0.5 g, 2.35 mmol) in THF (5 mL) dropwise at −78° C. under N₂. After 30 min, a solution of 1-(4-bromophenyl)-2,2,2-trifluoro-ethanone (0.54 mL, 3.5 mmol) in THF (5 mL) was added. The reaction was allowed to stir at −78° C. for an additional 2 h. After completion, the reaction was poured into water (10 mL) and extracted with EtOAc (10 mL×3). The combined organic layer was washed with brine, dried over anhydrous Na₂SO₄, filtered and concentrated. The crude residue was purified by silica gel column chromatography (0-100% EtOAc/PE) to afford the title product (0.6 g, 54.8%) as a yellow oil. MS (ESI): mass calcd, for C₁₈H₂₄BrF₃N₂O₂Si: 464.07, found: 465.2 [M+H]⁺.

### 1-(4-Aminophenyl)-2,2,2-trifluoro-1-(5-methyl-1-((2-(trimethylsilyl)ethoxy)methyl)-1H-imidazol-2-yl) ethan-1-ol

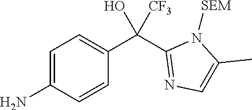 [SMILES: Cc1cnc(C(O)(c2ccc(N)cc2)C(F)(F)F)n1COCC[Si](C)(C)C]

To a mixture of 1-(4-bromophenyl)-2,2,2-trifluoro-1-[5-methyl-1-(2-trimethylsilylethoxymethyl) imidazol-2-yl]ethanol (0.6 g, 1.3 mmol) in EtOH (10 mL) and H₂O (2 mL) was added CuSO₄ (205.8 mg, 1.29 mmol), N1,N2-dimethylcyclohexane-1,2-diamine (110 mg, 0.77 mmol), sodium ascorbate (510.8 mg, 2.6 mmol) and NaN₃ (0.13 g, 2 mmol) at 25° C. under N₂. After heating at 80° C. for 12 h, the reaction was poured into ice-water (10 mL), adjusted to pH=9-10 by Na₂CO₃ and extracted with EtOAc (10 mL×3). The combined organic layer was washed with brine, dried with anhydrous Na₂SO₄, filtered and concentrated. The crude residue was purified by silica gel column chromatography (PE:EtOAc=1:0 to 0:1) to provide the title compound (0.15 g, 29%) as a yellow solid. MS (ESI): mass calcd, for C₁₈H₂₆F₃N₃O₂Si: 401.17, found: 402.3 [M+H]⁺.

### 1-(2-Aminobenzo[d]thiazol-6-yl)-2,2,2-trifluoro-1-(5-methyl-1-((2-(trimethylsilyl)ethoxy)methyl)-1H-imidazol-2-yl) ethan-1-ol

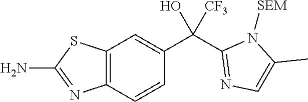 [SMILES: Cc1cnc(C(O)(c2ccc3nc(N)sc3c2)C(F)(F)F)n1COCC[Si](C)(C)C]

To a solution of 1-(4-aminophenyl)-2,2,2-trifluoro-1-[5-methyl-1-(2-trimethylsilylethoxymethyl) imidazol-2-yl]ethanol (0.133 g, 0.33 mmol) in AcOH (1 mL) was added KSCN (112.7 mg, 1.16 mmol) at 25° C. After stirring at this temperature for 1 h, Br₂ (58.2 mg, 0.36 mmol) was added. The resulting mixture was stirred for another 3 h, quenched with saturated NaHCO₃ (3 mL) and extracted with EtOAc (3 mL×3). The combined organic layer was washed with brine, dried with anhydrous Na₂SO₄, filtered and concentrated. The crude residue was purified by prep-TLC (PE:EtOAc=1:1) to afford the title compound (0.097 g, with impurity) as a yellow oil. MS (ESI): mass calcd, for C₁₉H₂₅F₃N₄O₂SSi: 458.14, found: 459.2 [M+H]⁺.

### 1-(4-Fluorophenyl)-3-(6-(2,2,2-trifluoro-1-hydroxy-1-(5-methyl-1-((2-(trimethylsilyl)ethoxy)methyl)-1H-imidazol-2-yl)ethyl)benzo[d]thiazol-2-yl)urea

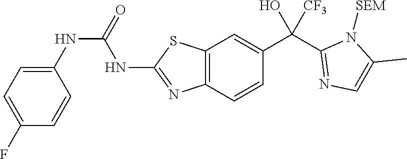 [SMILES: Cc1cnc(C(O)(c2ccc3nc(NC(=O)Nc4ccc(F)cc4)sc3c2)C(F)(F)F)n1COCC[Si](C)(C)C]

To a mixture of 1-(2-aminobenzo[d]thiazol-6-yl)-2,2,2-trifluoro-1-(5-methyl-1-((2-(trimethylsilyl)ethoxy)methyl)-1H-imidazol-2-yl) ethan-1-ol (0.097 g, 0.21 mmol) in DMF (1 mL) was added 1-fluoro-4-isocyanatobenzene (34.8 mg, 0.25 mmol) at 0° C. under N₂. After stirring at 25° C. for 4 h, the reaction was quenched with water (3 mL) and extracted with EtOAc (3 mL×3). The combined organic layer was washed with brine, dried with anhydrous Na₂SO₄, filtered and concentrated. The crude residue was purified by prep-TLC (PE:Ethyl acetate=1:1) to provide the title compound (0.074 g, with impurity) as a yellow powder. MS (ESI): mass calcd, for CH₂₉F₄N₅O₃SSi: 595.17, found: 596.3 [M+H]⁺.

### 1-(4-Fluorophenyl)-3-(6-(2,2,2-trifluoro-1-hydroxy-1-(5-methyl-1H-imidazol-2-yl)ethyl)benzo[d]thiazol-2-yl)urea (94)

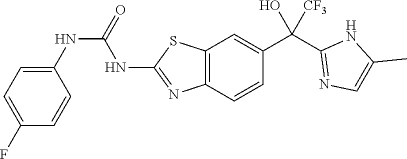 [SMILES: Cc1cnc(C(O)(c2ccc3nc(NC(=O)Nc4ccc(F)cc4)sc3c2)C(F)(F)F)[nH]1]

A mixture of 1-(4-fluorophenyl)-3-(6-(2,2,2-trifluoro-1-hydroxy-1-(5-methyl-1-((2-(trimethylsilyl)ethoxy)methyl)-1H-imidazol-2-yl)ethyl)benzo[d]thiazol-2-yl)urea (0.074 g, 0.12 mmol) in TFA (2 mL) and water (0.4 mL) was stirred at 25-40° C. for 6 h. The reaction was poured into saturated NaHCO₃ (5 mL) and extracted with EtOAc (3 mL×3). The combined organic layer was washed with brine, dried with anhydrous Na₂SO₄, filtered and concentrated. The crude product was purified by reversed-phase HPLC (mobile phase: [H₂O (10 mM NH₄HCO₃)-ACN]; gradient: 30-50% B) to provide the title compound (94) (8.7 mg, racemic) as a white solid. MS (ESI): mass calcd, for C₂₀H₁₅F₄N₅O₂S: 465.09, found: 466.1 [M+H]⁺. ¹H NMR (400 MHz, DMSO-d₆) δ ppm 11.76-11.89 (m, 1H), 10.86 (s, 1H), 9.17-9.30 (m, 1H), 8.15 (s, 1H), 7.53-7.65 (m, 5H), 7.17 (t, J=8.4 Hz, 2H), 6.62-6.80 (m, 1H), 2.13 (d, J=5.2 Hz, 3H).

Examples 95, 96, 97, 98, 99, and 100 were synthesized in similar procedures as described in Example 38.

### Example 101. (S)-3,3,3-Trifluoro-2-(2-(3-(4-fluorophenyl)ureido)benzo[d]thiazol-6-yl)-2-hydroxy-N—((S)-pyrrolidin-3-yl)propenamide (101)

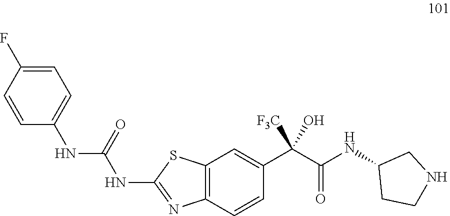 [SMILES: O=C(Nc1ccc(F)cc1)Nc1nc2ccc([C@](O)(C(=O)N[C@H]3CCNC3)C(F)(F)F)cc2s1]

### Synthetic Scheme:

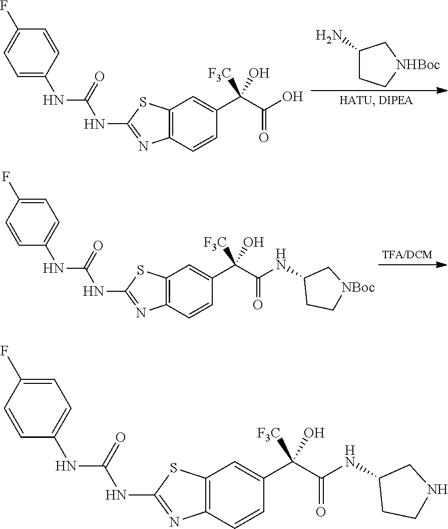

### tert-Butyl (S)-3-((S)-3,3,3-trifluoro-2-(2-(3-(4-fluorophenyl)ureido)benzo[d]thiazol-6-yl)-2-hydroxypropanamido) pyrrolidine-1-carboxylate

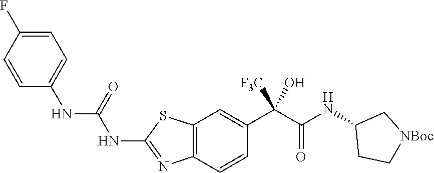 [SMILES: CC(C)(C)OC(=O)N1CC[C@H](NC(=O)[C@@](O)(c2ccc3nc(NC(=O)Nc4ccc(F)cc4)sc3c2)C(F)(F)F)C1]

The title compound was synthesized from ethyl 3,3,3-trifluoro-2-hydroxy-2-(2-(3-phenylureido)quinolin-6-yl)propanoate in similar procedures as described in Example 38. MS (ESI): mass calcd, for C₂₆H₂₇F₄N₅O₅S: 597.17, found: 620.0 [M+Na]⁺.

### (S)-3,3,3-Trifluoro-2-(2-(3-(4-fluorophenyl)ureido)benzo[d]thiazol-6-yl)-2-hydroxy-N—((S)-pyrrolidin-3-yl)propenamide (101)

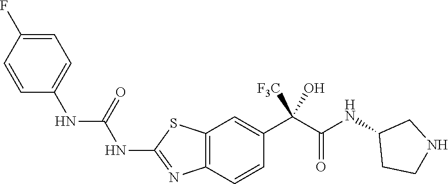 [SMILES: O=C(Nc1ccc(F)cc1)Nc1nc2ccc([C@](O)(C(=O)N[C@H]3CCNC3)C(F)(F)F)cc2s1]

A solution of ethyl tert-butyl (S)-3-((S)-3,3,3-trifluoro-2-(2-(3-(4-fluorophenyl)ureido)benzo[d]thiazol-6-yl)-2-hydroxypropanamido) pyrrolidine-1-carboxylate (0.04 g, 0.067 mmol) in DCM (1 mL) and TFA (0.2 mL) was stirred at 25° C. for 1 h. After completion, the reaction was quenched with sat. NaHCO₃ and adjusted to pH=7. The aqueous layer was extracted with EtOAc (3 mL×3). The combined organic layer was washed with brine, dried over Na₂SO₄, filtered, and concentrated. The resulting residue was purified by reverse phase HPLC (15-40% ACN in H₂O (10 mM NH₄HCO₃), gradient elution) to provide the title compound (101) (1.7 mg, 5.1%) as a white solid. MS (ESI): mass calcd. for C₂₁H₁₉F₄N₅O₃S: 497.11, found: 498.1 [M+H]⁺. ¹H NMR (400 MHz, DMSO-d₆) δ ppm 9.21 (s, 1H), 8.06-8.12 (m, 2H), 7.73-7.75 (m, 1H), 7.54-7.58 (m, 4H), 7.13 (t, J=8.8 Hz, 2H), 4.16-4.19 (m, 1H), 2.83-2.92 (m, 2H), 2.66-2.70 (m, 2H), 2.32-2.33 (m, 1H), 1.88-1.91 (m, 1H), 1.46-1.48 (m, 1H).

Examples 102, 155, 169, and 181 were synthesized in similar procedures as described in Example 101.

Examples 103, 104, 105, 106, 107, 109 (from Example 92), 110, 111, and 112 were synthesized in similar procedures as described in Example 38.

Example 108 was synthesized in similar procedures as described in Example 36,

### Example 113. N-Ethyl-4,4,4-trifluoro-2-(2-(3-(4-fluorophenyl)ureido)benzo[d]thiazol-6-yl)-2-hydroxybutanamide (113)

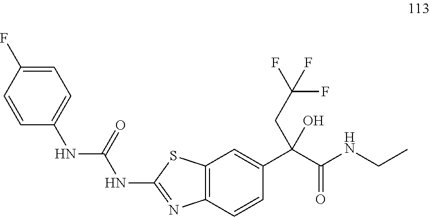 [SMILES: CCNC(=O)C(O)(CC(F)(F)F)c1ccc2nc(NC(=O)Nc3ccc(F)cc3)sc2c1]

### Synthetic Scheme:

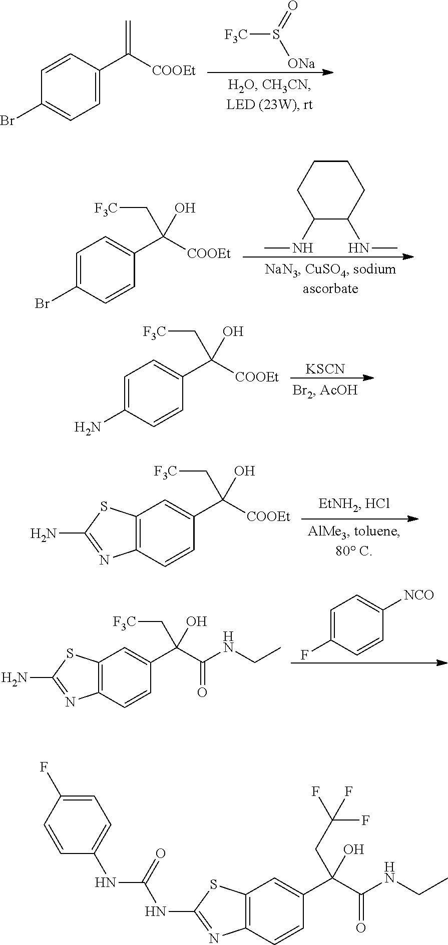

### Ethyl 2-(4-bromophenyl)-4,4,4-trifluoro-2-hydroxybutanoate

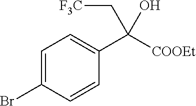 [SMILES: CCOC(=O)C(O)(CC(F)(F)F)c1ccc(Br)cc1]

TFA (0.58 mL, 7.84 mmol) was added to a solution of ethyl 2-(4-bromophenyl) prop-2-enoate (2 g, 7.84 mmol) and sodium trifluoromethanesulfonate (2.45 g, 15.7 mmol) in DMSO (20 mL) at 40° C. After stirring at the same temperature for 48 h, the reaction was poured into water (30 mL) and extracted with EtOAc (30 mL×3). The combined organic layer was washed with brine, dried over Na₂SO₄, filtered and concentrated. The resulting residue was purified by silica gel column chromatography (0-100% EtOAc/PE) to afford the title compound (0.4 g, with impurity) as a yellow solid.

### Ethyl 2-(4-aminophenyl)-4,4,4-trifluoro-2-hydroxy-butanoate

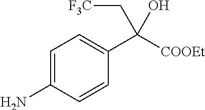 [SMILES: CCOC(=O)C(O)(CC(F)(F)F)c1ccc(N)cc1]

To a mixture of ethyl 2-(4-bromophenyl)-4,4,4-trifluoro-2-hydroxy-butanoate (0.4 g, 1.17 mmol), CuSO₄ (187.2 mg, 1.17 mmol), N1,N2-dimethylcyclohexane-1,2-diamine (100.1 mg, 0.70 mmol) and sodium ascorbate (464.6 mg, 2.35 mmol) in EtOH (5 mL) and H₂O (1 mL) was added NaN₃ (0.3 g, 4.61 mmol) at 20° C. under N₂. After heating at 80° C. for 12 h, the reaction was poured into ice-water (10 mL), adjusted to pH=9-10 with aq. Na₂CO₃ and extracted with ethyl acetate (10 mL×3). The combined organic layer was washed with brine, dried over Na₂SO₄, filtered and concentrated. The crude residue was purified by silica gel column chromatography (PE:EtOAc=1:0 to 0:1) to afford the title compound (0.1 g, 30.8%) as a yellow solid. MS (ESI): mass calcd, for C₁₂H₁₄F₃NO₃: 277.09, found: 278.3 [M+H]⁺.

### N-Ethyl-4,4,4-trifluoro-2-(2-(3-(4-fluorophenyl)ureido)benzo[d]thiazol-6-yl)-2-hydroxybutanamide (113)

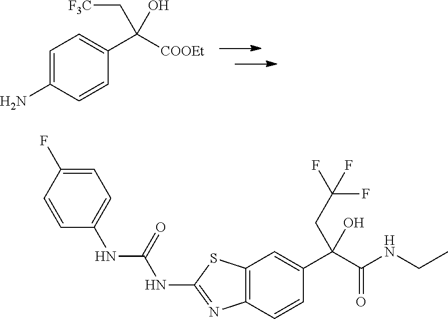

The title compound (113) (racemic) was synthesized from ethyl 2-(4-aminophenyl)-4,4,4-trifluoro-2-hydroxy-butanoate in similar procedures as described in Example 61. MS (ESI): mass calcd, for C₂₀H₁₈F₄N₄O₃S: 470.10, found: 471.1 [M+H]⁺. ¹H NMR (400 MHz, DMSO-d₆) δ ppm 10.59-10.98 (m, 1H), 9.25 (s, 1H), 7.95-8.14 (m, 2H), 7.55 (d, J=6.8 Hz, 4H), 7.17 (t, J=8.8 Hz, 2H), 6.58 (s, 1H), 3.38-3.53 (m, 1H), 2.94-3.14 (m, 2H), 2.79-2.92 (m, 1H), 0.95 (t, J=7.2 Hz, 3H).

### Example 114. N-Ethyl-3,3,3-trifluoro-2-(2-(3-(4-fluorophenyl)ureido)benzo[d]thiazol-6-yl)-2-methoxypropanamide (114)

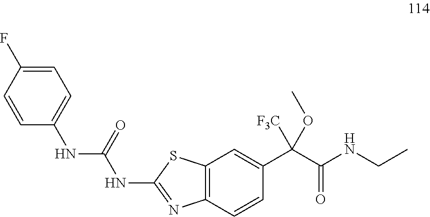 [SMILES: CCNC(=O)C(OC)(c1ccc2nc(NC(=O)Nc3ccc(F)cc3)sc2c1)C(F)(F)F]

### Synthetic Scheme:

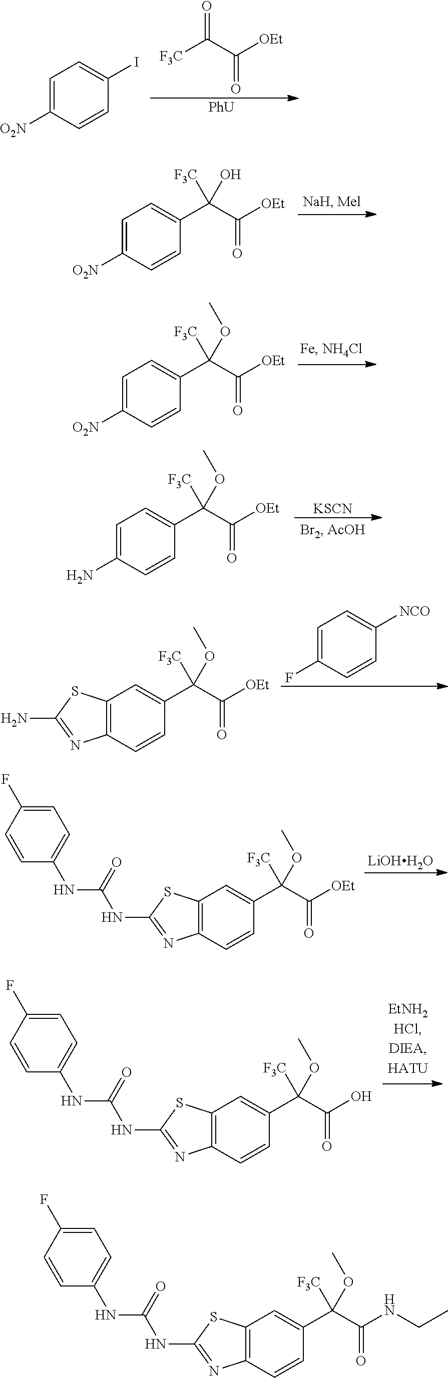

### Ethyl 3,3,3-trifluoro-2-hydroxy-2-(4-nitrophenyl)propanoate

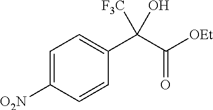 [SMILES: CCOC(=O)C(O)(c1ccc([N+](=O)[O-])cc1)C(F)(F)F]

To a solution of 1-iodo-4-nitro-benzene (5 g, 20.1 mmol) in THF (50 mL) was added 1 M phenyllithium (30.1 mL, 30.1 mmol) dropwise at −70° C. After stirring at this temperature for 30 min, ethyl 3,3,3-trifluoro-2-oxo-propanoate (3.2 mL, 24.1 mmol) was added and stirred at −70° C. for an additional 2 h. After completion, the reaction was quenched with sat. NH₄Cl (100 mL) and extracted with EtOAc (100 mL×3). The combined organic layer was washed with brine, dried over Na₂SO₄, filtered and concentrated under reduced pressure. The resulting residue was purified by silica gel column chromatography (0-20% EtOAc/PE) to afford the title compound (1.9 g, 32.3%) as a brown oil. ¹H NMR (400 MHz, DMSO-d₆) δ ppm 8.28-8.33 (m, 3H), 7.87 (d, J=8.8 Hz, 2H), 4.26-4.32 (m, 2H), 1.22 ((, J=7.2 Hz, 3H).

### Ethyl 3,3,3-trifluoro-2-methoxy-2-(4-nitrophenyl)propanoate

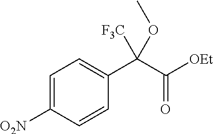 [SMILES: CCOC(=O)C(OC)(c1ccc([N+](=O)[O-])cc1)C(F)(F)F]

To an ice cold solution of ethyl 3,3,3-trifluoro-2-hydroxy-2-(4-nitrophenyl)propanoate (1 g, 3.4 mmol) in DMF (10 mL) was added 60% NaH (204.6 mg, 5.1 mmol) and MeI (0.42 mL, 6.8 mmol). After stirring for 12 h, the reaction was quenched with sat. NH₄Cl (100 mL) and extracted with EtOAc (100 mL×3). The combined organic layer was washed with brine, dried over Na₂SO₄, filtered and concentrated under reduced pressure. The resulting residue was purified by silica gel column chromatography (0-5% EtOAc/PE) to afford the title compound (700 mg, 70%) as a brown oil. ¹H NMR (400 MHz, DMSO-d₆) δ ppm 8.30-8.33 (m, 2H), 7.78 (d, J=8.8 Hz, 2H), 4.35-4.43 (m, 2H), 3.55 (s, 3H), 1.25 (t, J=7.2 Hz, 3H)

### Ethyl 2-(4-aminophenyl)-3,3,3-trifluoro-2-methoxypropanoate

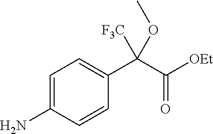 [SMILES: CCOC(=O)C(OC)(c1ccc(N)cc1)C(F)(F)F]

Fe (636.2 mg, 11.4 mmol) and NH₄Cl (609.4 mg, 11.4 mmol) were added to a solution of ethyl 3,3,3-trifluoro-2-methoxy-2-(4-nitrophenyl)propanoate (700 mg, 2.3 mmol) in EtOH (7 mL) and H₂O (7 mL) at rt. After heating at 80° C. for 2 h, the reaction was diluted with water (10 mL) and extracted with EtOAc (10 mL×3). The combined organic layer was washed with brine, dried over anhydrous Na₂SO₄, filtered and concentrated. The resulting residue was purified by silica gel column chromatography (0-95% EtOAc/PE) to afford the title compound (600 mg, 95%) as a brown oil. ¹H NMR (400 MHz, DMSO-d₆) δ ppm 7.05 (d, J=8.4 Hz, 2H), 6.56-6.60 (m, 2H), 5.46 (s, 2H), 4.30-4.36 (m, 2H), 3.37 (s, 3H), 1.25 (t, J=7.2 Hz, 3H).

### Ethyl 3,3,3-trifluoro-2-(2-(3-(4-fluorophenyl)ureido)benzo[d]thiazol-6-yl)-2-methoxypropanoate

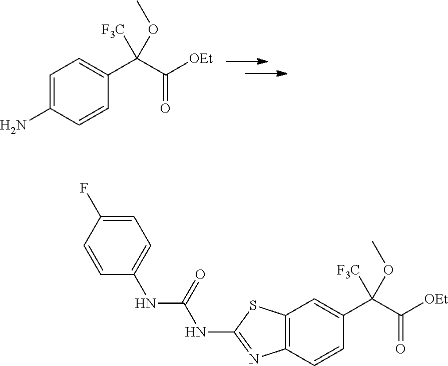 [SMILES: CCOC(=O)C(OC)(c1ccc(N)cc1)C(F)(F)F ; CCOC(=O)C(OC)(c1ccc2nc(NC(=O)Nc3ccc(F)cc3)sc2c1)C(F)(F)F]

The title compound was synthesized from ethyl 2-(4-aminophenyl)-3,3,3-trifluoro-2-methoxypropanoate in similar procedures as described in Example 41. MS (ESI): mass calcd. for C₂₀H₁₇F₄N₃O₄S: 471.09, found: 472.1 [M+H]⁺.

### N-Ethyl-3,3,3-trifluoro-2-(2-(3-(4-fluorophenyl)ureido)benzo[d]thiazol-6-yl)-2-methoxypropanamide (114)

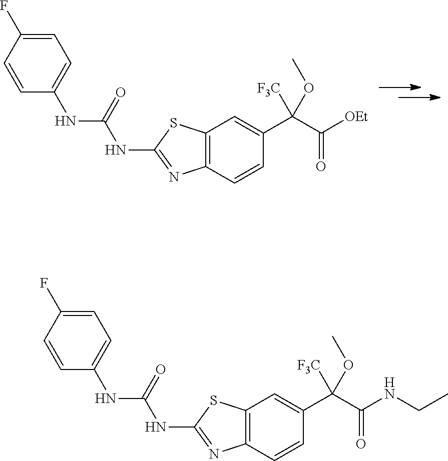 [SMILES: CCOC(=O)C(OC)(c1ccc2nc(NC(=O)Nc3ccc(F)cc3)sc2c1)C(F)(F)F ; CCNC(=O)C(OC)(c1ccc2nc(NC(=O)Nc3ccc(F)cc3)sc2c1)C(F)(F)F]

The title compound (114) was synthesized from ethyl 3,3,3-trifluoro-2-(2-(3-(4-fluorophenyl)ureido)benzo[d]thiazol-6-yl)-2-methoxypropanoate in similar procedures (last 2 steps) as described in Example 38. MS (ESI): mass calcd, for C₂₀H₁₈F₄N₄O₃S: 470.10. found: 471.1 [M+H]⁺. ¹H NMR (400 MHz, DMSO-d₆) δ ppm 11.01 (s, 1H), 9.26 (s, 1H), 8.40 (t, J=5.6 Hz, 1H), 8.07 (s, 1H), 7.67 (d, J=8.4 Hz, 1H), 7.52-7.56 (m, 2H), 7.44-7.47 (m, 1H), 7.17 (t, J=8.8 Hz, 2H), 3.45 (s, 3H), 3.14-3.26 (m, 2H), 1.03 (t, J=7.2 Hz, 3H).

Example 115 was synthesized in similar procedures as described in Example 38.

### Example 116. N-Ethyl-3,3,3-trifluoro-2-(2-(3-(4-fluorophenyl)ureido)-4-methylbenzo[d]thiazol-6-yl)-2-hydroxypropanamide (116)

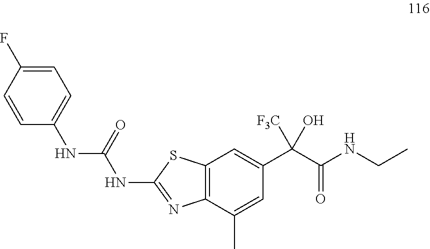 [SMILES: CCNC(=O)C(O)(c1cc(C)c2nc(NC(=O)Nc3ccc(F)cc3)sc2c1)C(F)(F)F]

### Synthetic Scheme:

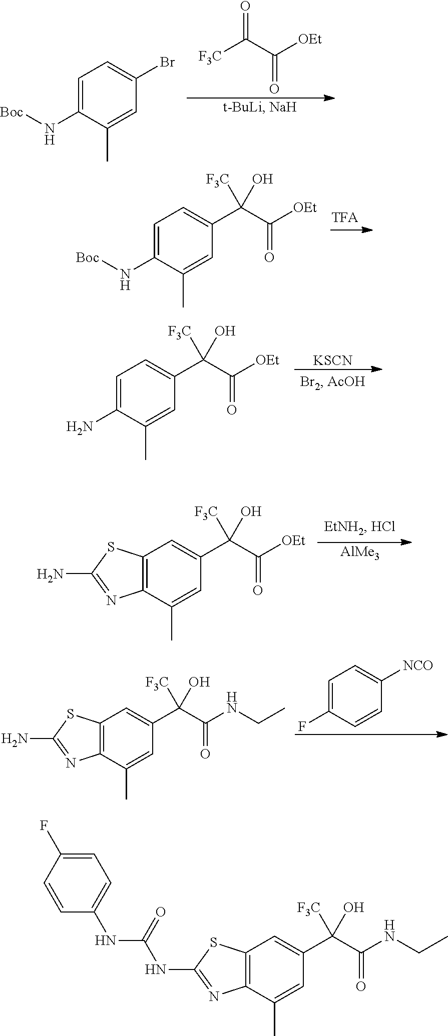

### 2-(2-Amino-4-methylbenzo[d]thiazol-6-yl)-N-ethyl-3,3,3-trifluoro-2-hydroxypropanamide

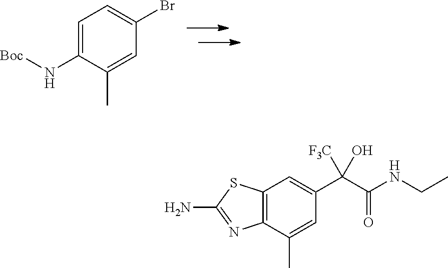 [SMILES: Cc1cc(Br)ccc1NC(=O)OC(C)(C)C ; CCNC(=O)C(O)(c1cc(C)c2nc(N)sc2c1)C(F)(F)F]

The title compound was synthesized from tert-butyl (4-bromo-2-methylphenyl)carbamate in similar procedures as described in Example 6, steps 1-3 and Example 34, step 1. MS (ESI): mass calcd, for C₁₃F₁₄N₃O₂S: 333.08, found: 334.1 [M+H]⁺.

### N-Ethyl-3,3,3-trifluoro-2-(2-(3-(4-fluorophenyl)ureido)-4-methylbenzo[d]thiazol-6-yl)-2-hydroxypropanamide (116)

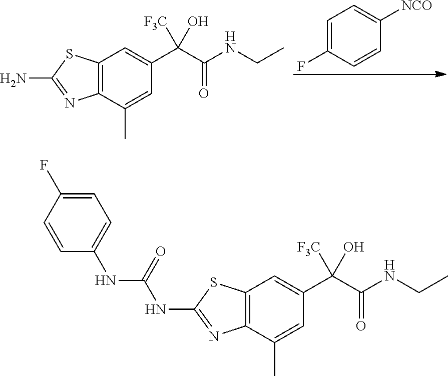

To a solution of 2-(2-amino-4-methylbenzo[d]thiazol-6-yl)-N-ethyl-3,3,3-trifluoro-2-hydroxypropanamide (0.1 g, 0.3 mmol) in DMF (2 mL) was added 1-fluoro-4-isocyanato-benzene (49.4 mg, 0.36 mmol) at 0° C. After stirring at 25° C. for 2 h, the reaction was diluted with saturated NH₄Cl (15 mL) and extracted with EtOAc (25 mL×3). The combined organic layer was washed with brine, dried over Na₂SO₄, filtered and concentrated under reduced pressure. The crude residue was purified by reverse phase HPLC (column: Phenomenex Luna C18 75*30 mm*3 μm; mobile phase: [H₂O (0.2% FA)-ACN]; gradient elution: 35-65% B) to provide the title compound (116) (13.7 mg, 9.7%, racemic) as a white solid. MS (ESI): mass calcd, for C₂₀H₁₈F₄N₄O₃S: 470.10, found: 471.1 [M+H]⁺. ¹H NMR (400 MHz, DMSO-d6) δ ppm 11.15 (d, J=2.4 Hz, 1H), 9.10 (s, 1H), 8.17 (t, J=6.0 Hz, 1H), 7.99 (s, 1H), 7.71 (s, 1H), 7.49-7.54 (m, 3H), 7.18 (t, J=8.8 Hz, 2H), 3.08-3.16 (m, 2H), 2.55 (s, 3H), 0.99 (t, J=7.2 Hz, 3H).

Examples 120, 121, 142, 158, and 186 were synthesized in similar procedures as described in Example 116.

Examples 117 was synthesized in similar procedures as described in Example 60.

### Example 119. 2-Cyclopropyl-N-ethyl-2-(2-(3-(4-fluorophenyl)ureido)benzo[d]thiazol-6-yl)-2-hydroxyacetamide (119)

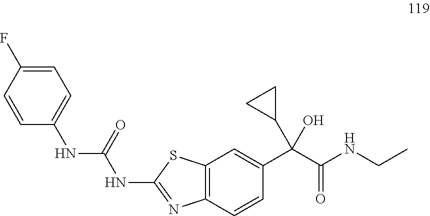 [SMILES: CCNC(=O)C(O)(c1ccc2nc(NC(=O)Nc3ccc(F)cc3)sc2c1)C1CC1]

### Synthetic Scheme:

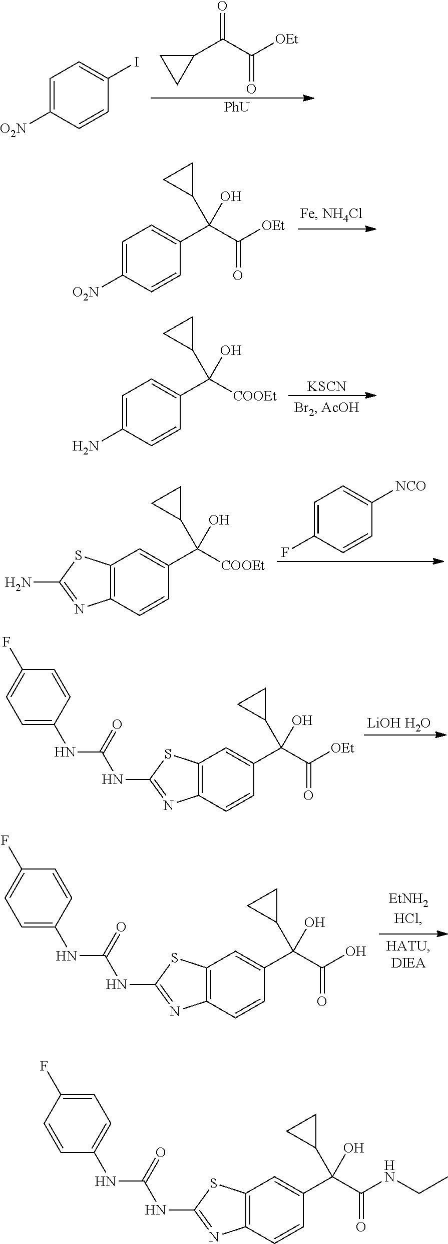

### Ethyl 2-cyclopropyl-2-hydroxy-2-(4-nitrophenyl)acetate

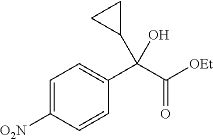 [SMILES: CCOC(=O)C(O)(c1ccc([N+](=O)[O-])cc1)C1CC1]

To a solution of 1-iodo-4-nitro-benzene (2 g, 8.03 mmol) in THF (90 mL) was added 1 M PhLi (8.0 mL, 8.0 mmol) dropwise at −78° C. After stirring for 30 min at −78° C., ethyl 2-cyclopropyl-2-oxo-acetate (1.37 g, 9.64 mmol) was added, and the reaction was stirred for an additional 3 h at the same temperature. The reaction was quenched with sat. NH₄Cl solution (90 mL) and extracted with EtOAc (90 mL×2). The combined organic layer was washed with brine, dried over Na₂SO₄, filtered and concentrated under reduced pressure. The crude residue was purified by silica gel column chromatography (0-50% EtOAc/PE) to afford the title compound (1.4 g, 65.7%) as a yellow solid. ¹H NMR (400 MHz, DMSO-d₆) δ ppm 8.22 (d, J=8.8 Hz, 2H), 7.82 (d, J=9.2 Hz, 2H), 4.09-4.18 (m, 2H), 1.66-1.74 (m, 1H). 1.15-1.19 (m, 3H), 0.59-0.68 (m, 1H), 0.30-0.47 (m, 3H).

### 2-Cyclopropyl-N-ethyl-2-(2-(3-(4-fluorophenyl)ureido)benzo[d]thiazol-6-yl)-2-hydroxyacetamide (119)

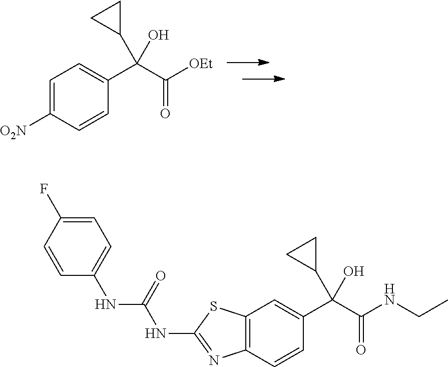 [SMILES: CCOC(=O)C(O)(c1ccc([N+](=O)[O-])cc1)C1CC1 ; CCNC(=O)C(O)(c1ccc2nc(NC(=O)Nc3ccc(F)cc3)sc2c1)C1CC1]

The title compound (119) (racemic) was synthesized from ethyl 2-cyclopropyl-2-hydroxy-2-(4-nitrophenyl)acetate in similar procedures (last 5 steps) as described in Example 114. MS (ESI): mass calcd, for C₂₁H₂₃N₅O₂S: 428.13, found: 429.1 [M+H]⁺. ¹H NMR (400 MHz, DMSO-d₆) δ ppm 10.73 (s, 1H), 9.25 (s, 1H), 8.05 (s, 1H), 7.87 (t, J=6.0 Hz, 1H), 7.47-7.65 (m, 4H), 7.17 (t, J=8.8 Hz, 2H), 5.52 (s, 1H), 3.07-3.14 (m, 2H), 1.70-1.79 (m, 1H), 1.01 (t, J=7.2 Hz, 3H), 0.44-0.55 (m, 3H), 0.31-0.41 (m, 1H).

### Example 122. (S)-1-(4-Fluorophenyl)-3-(6-(2,2,2-trifluoro-1-hydroxy-1-(5-methyl-4H-1,2,4-triazol-3-yl)ethyl)benzo[d]thiazol-2-yl)urea (122)

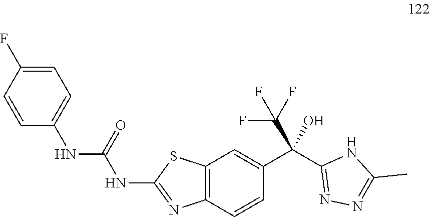 [SMILES: Cc1nnc([C@@](O)(c2ccc3nc(NC(=O)Nc4ccc(F)cc4)sc3c2)C(F)(F)F)[nH]1]

### Synthetic Scheme:

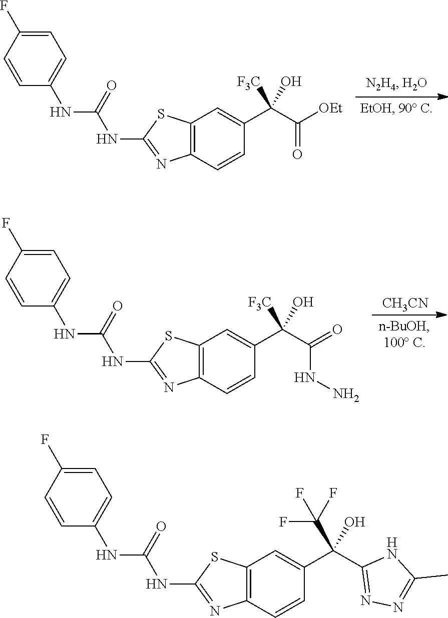

### (S)-1-(4-Fluorophenyl)-3-(6-(1,1,1-trifluoro-3-hydrazineyl-2-hydroxy-3-oxopropan-2-yl)benzo[d]thiazol-2-yl)urea

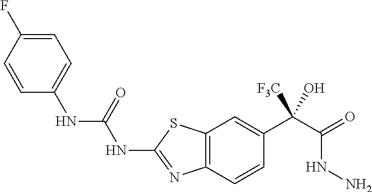 [SMILES: NNC(=O)[C@@](O)(c1ccc2nc(NC(=O)Nc3ccc(F)cc3)sc2c1)C(F)(F)F]

To a solution of ethyl (2S)-3,3,3-trifluoro-2-[2-[(4-fluorophenyl) carbamoylamino]-1,3-benzothiazol-6-yl]-2-hydroxy-propanoate (0.2 g, 0.44 mmol) in EtOH (2 mL) was added hydrazine hydrate (0.42 mL, 0.88 mmol) in one portion. After heating at 80° C. for 12 h, the reaction was poured into water (10 mL) and extracted with EtOAc (10 mL×3). The combined organic layer was washed with brine, dried over anhydrous Na₂SO₄, filtered and concentrated. The resulting residue was purified by prep-TLC (100% EtOAc) to afford the title compound (0.17 g, 85%) as a white solid. MS (ESI): mass calcd, for C₁₇H₁₃F₄N₅O₃S: 443.07, found: 444.2 [M+H]⁺.

### (S)-1-(4-Fluorophenyl)-3-(6-(2,2,2-trifluoro-1-hydroxy-1-(5-methyl-4H-1,2,4-triazol-3-ylethyl)benzo[d]thiazol-2-yl)urea (122)

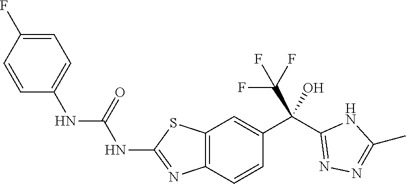 [SMILES: Cc1nnc([C@@](O)(c2ccc3nc(NC(=O)Nc4ccc(F)cc4)sc3c2)C(F)(F)F)[nH]1]

To a solution of 1-(4-fluorophenyl)-3-[6-[(1S)-2,2,2-trifluoro-1-(hydrazinecarbonyl)-1-hydroxy-ethyl]-1,3-benzothiazol-2-yl]urea (0.15 g, 0.34 mmol) in toluene (2 mL) and NMP (1 mL) was added TEA (1 mL, 7.18 mmol) and ethyl ethanimidate hydrochloride (334.5 mg, 2.7 mmol). After heating at 100° C. for 12 h, the reaction was poured into water (10 mL) and extracted with EtOAc (10 mL×3). The combined organic layer was washed with brine, dried over anhydrous Na₂SO₄, filtered and concentrated. The resulting residue was purified by reverse phase HPLC (20-45% ACN in H₂O (10 mM NH₄HCO₃), gradient elution) to provide the title compound (122) (7.2 mg, 4.6%) as a white solid. MS (ESI): mass calcd. for C₁₉H₁₄F₄N₆O₂S: 466.08, found: 467.0 [M+H]⁺. ¹H NMR (400 MHz, DMSO-d₆) δ ppm 9.71 (s, 1H), 8.05 (s, 1H), 7.50-7.69 (m, 5H), 7.34-7.38 (m, 1H), 7.16 (t, J=8.8 Hz, 2H), 2.37 (s, 3H).

Examples 123, 124, 125, 126, 127, 128, and 129 were synthesized in similar procedures as described in Example 38.

Examples 130 and 131 were synthesized in similar procedures as described in Example 67.

Examples 132 was synthesized in similar procedures as described in Example 109.

Examples 133 was synthesized in similar procedures as described in Example 1.

Examples 134 was synthesized in similar procedures as described in Example 40.

Examples 135 and 136 were synthesized in similar procedures as described in Example 38.

### Example 137. (S)-1-(6-(3-(Azetidin-1-yl)-1,1,1-trifluoro-2-hydroxy-3-oxopropan-2-yl)benzo[d]thiazol-2-yl)-3-(4-fluorophenyl)urea (137)

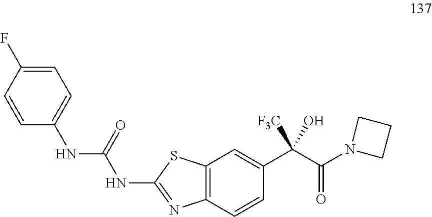 [SMILES: O=C(Nc1ccc(F)cc1)Nc1nc2ccc([C@](O)(C(=O)N3CCC3)C(F)(F)F)cc2s1]

### Synthetic Scheme:

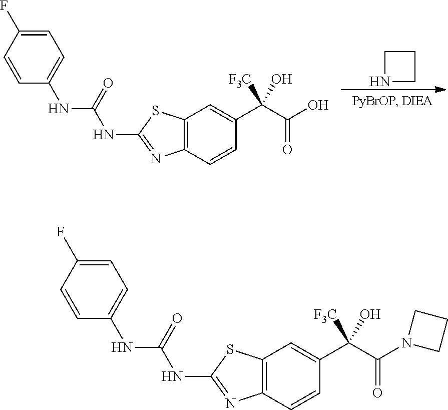

DIPEA (81.1 μL. 0.47 mmol) and PyBrOP (135.7 mg, 0.29 mmol) were added to a solution of (2S)-3,3,3-trifluoro-2-[2-[(4-fluorophenyl) carbamoylamino]-1,3-benzothiazol-6-yl]-2-hydroxy-propanoic acid (50 mg, 0.12 mmol) in THF (2 mL) at rt. After stirring for 10 min, azetidine (13.3 mg, 0.23 mmol) was added. The reaction was stirred for an additional 2 h, quenched with H₂O (2 mL) and extracted with EtOAc (2 mL×2). The combined organic layer was washed with brine, dried over Na₂SO₄, filtered, and concentrated. The resulting residue was purified by reverse phase HPLC (20-50% ACN in H₂O (10 mM NH₄HCO₃), gradient elution) to provide the title compound (137) (2.2 mg, 4.03%) as a white solid. MS (ESI): mass calcd, for C₂H₁₆F₄N₄O₃S: 468.09, found: 469.0 [M+H]⁺. ¹H NMR (400 MHz, DMSO-d₆) δ ppm 10.82-10.92 (m, 1H), 9.20-9.30 (m, 1H), 8.08 (s, 1H), 7.78 (s, 1H), 7.67-7.69 (m, 1H), 7.51-7.56 (m, 2H), 7.18 (t, J=8.8 Hz, 2H), 4.23-4.30 (m, 1H), 3.90 (t, J=8.0 Hz, 2H), 3.41-3.48 (m, 2H), 1.98-2.14 (m, 2H).

Examples 138, 139, 140, 143, 144, 145, and 157 were synthesized in similar procedures as described in Example 137.

### Example 146. Ethyl 3,3,3-trifluoro-2-(2-(3-(4-fluorophenyl)ureido)-1-methyl-1H-indol-5-yl)-2-hydroxypropanoate (146)

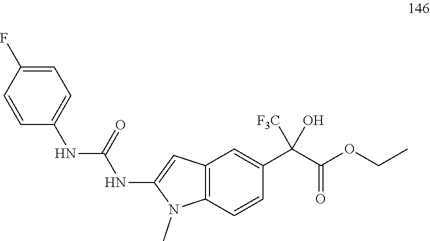 [SMILES: CCOC(=O)C(O)(c1ccc2c(c1)cc(NC(=O)Nc1ccc(F)cc1)n2C)C(F)(F)F]

### Synthetic Scheme:

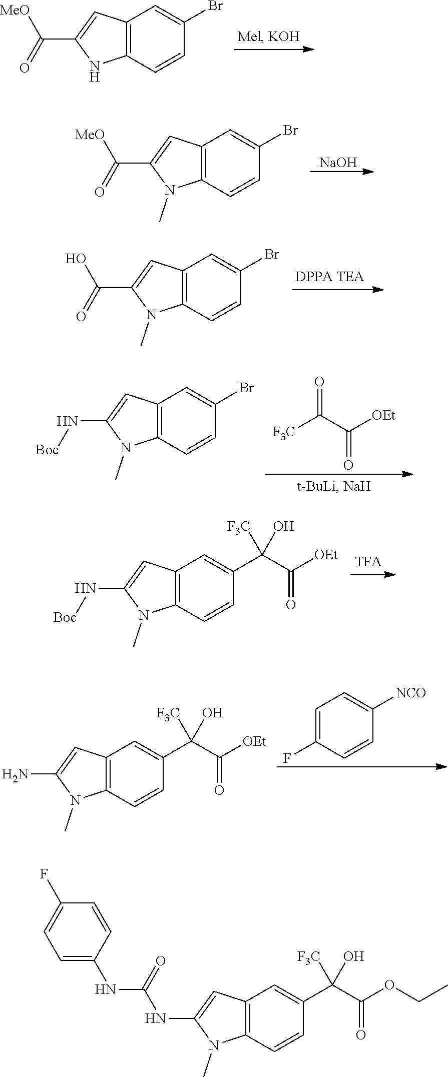

### Methyl 5-bromo-1-methyl-1H-indole-2-carboxylate

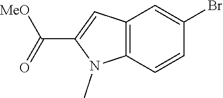

To a solution of methyl 5-bromo-1H-indole-2-carboxylate (3 g, 11.8 mmol) in DMF (25 mL) was added KOH (3.31 g, 59 mmol) and MeI (1.74 mL, 28 mmol). After stirring at rt for 1 h, the reaction was partitioned between EtOAc (100 mL) and H₂O (125 mL). The organic layer was separated, and the aqueous layer was extracted with EtOAc (100 mL×2). The combined organic layer was washed with brine, dried over Na₂SO₄, filtered, and concentrated under reduced pressure. The resulting residue was purified by silica gel column chromatography (0-20% EtOAc/PE) to afford the title compound (3 g, 93.8%) as a yellow solid. MS (ESI): mass calcd, for C₁₁H₁₀BrNO₂: 266.99, found: 268.0 [M+H]⁺.

### 5-Bromo-1-methyl-1H-indole-2-carboxylic Acid

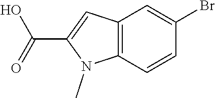

A suspension of methyl 5-bromo-1-methyl-indole-2-carboxylate (3 g, 11.2 mmol) and NaOH (1.79 g, 44.8 mmol) in EtOH/THE/H₂O (1:1:1, 30 mL) was stirred at rt for 4 h. After completion, the reaction was cooled down to 0° C. and acidified by conc. HCl to pH=2. The resulting precipitate was filtered and dried to afford the title compound (2.2 g, 76.6%) as a yellow solid. ¹H NMR (400 MHz, CDCl₃) δ ppm 7.85 (d, J=1.6 Hz, 1H), 7.45-7.48 (m, 1H), 7.38 (s, 1H), 7.30 (d, J=8.8 Hz, 1H), 4.09 (s, 3H).

### tert-Butyl (5-bromo-1-methyl-1H-indol-2-yl)carbamate

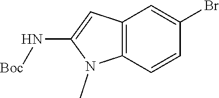 [SMILES: Cn1c(NC(=O)OC(C)(C)C)cc2cc(Br)ccc21]

TEA (2.41 mL, 17.32 mmol) and DPPA (2.8 mL, 12.99 mmol) were added to a stirred solution of 5-bromo-1-methyl-indole-2-carboxylic acid (2.2 g, 8.7 mmol) in tert-BuOH (95 mL) at rt under nitrogen. After heating at 120° C. for 12 h, the reaction was cooled down to rt and partitioned between EtOAc (100 mL) and H₂O (100 mL). The organic phase was separated, and the aqueous layer was extracted with EtOAc (100 mL×2). The combined organic layer was dried over Na₂SO₄, filtered, and concentrated. The resulting crude residue was purified by silica gel column chromatography (0-25% EtOAc/PB) to afford the title compound (2 g, 69.6%) as a yellow solid. MS (ESI): mass calcd, for C₁₄H₁₇BrN₂O₂: 324.05, found: 325.1 [M+H]⁺.

### Ethyl 3,3,3-trifluoro-2-(2-(3-(4-fluorophenyl)ureido)-1-methyl-1H-indol-5-yl)-2-hydroxypropanoate (146)

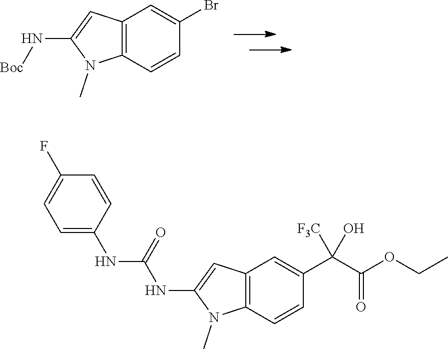 [SMILES: Cn1c(NC(=O)OC(C)(C)C)cc2cc(Br)ccc21 ; CCOC(=O)C(O)(c1ccc2c(c1)cc(NC(=O)Nc1ccc(F)cc1)n2C)C(F)(F)F]

The title compound (146) (racemic) was synthesized from tert-butyl (5-bromo-1-methyl-1H-indol-2-yl)carbamate in similar procedures as described in Example 1. MS (ESI): mass calcd, for C₂₁H₁₉F₄N₃O₄: 453.13, found: 454.1 [M+H]⁺. ¹H NMR (400 MHz, DMSO-d6) δ ppm 9.26 (s, 1H), 7.72-7.76 (m, 2H), 7.69 (d, J=8.0 Hz, 1H), 7.43-7.47 (m, 3H), 7.06-7.18 (m, 4H), 4.06-4.18 (m, 2H), 3.55 (s, 3H), 1.13 (t, J=7.2 Hz, 3H).

### Example 147. N-Ethyl-3,3,3-trifluoro-2-(2-(3-(4-fluorophenyl)ureido)thiazolo[5,4-b]pyridin-S-yl)-2-hydroxypropanamide (147)

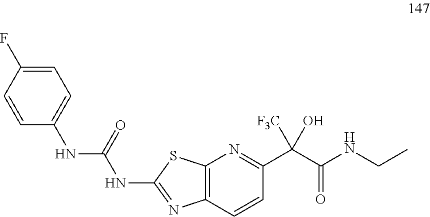 [SMILES: CCNC(=O)C(O)(c1ccc2nc(NC(=O)Nc3ccc(F)cc3)sc2n1)C(F)(F)F]

### Synthetic Scheme:

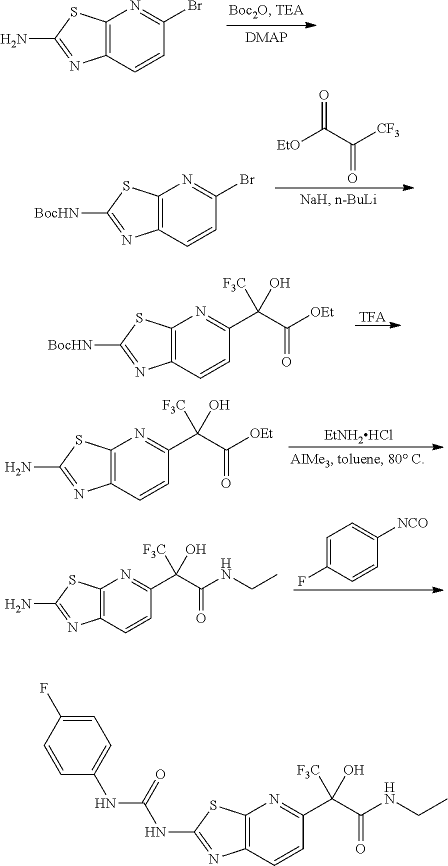

### tert-Butyl (5-bromothiazolo[5,4-b]pyridin-2-yl)carbamate

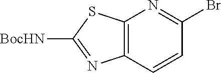 [SMILES: CC(C)(C)OC(=O)Nc1nc2ccc(Br)nc2s1]

To a solution of 5-bromothiazolo[5,4-b]pyridin-2-amine (1 g, 4.35 mmol) in THF (10 mL) was added TEA (786 μL, 5.65 mmol), Boc₂O (1.30 mL, 5.65 mmol) and DMAP (53.1 mg, 0.43 mmol). After stirring at rt for 5 h, the reaction was diluted with H₂O (10 mL) and extracted with EtOAc (15 mL×3). The combined organic layer was washed with brine, dried over Na₂SO₄, filtered and concentrated under reduced pressure. The crude product was triturated with DCM (10 mL) at rt for 10 min. The resulting solid was filtered and dried to afford the title compound (1.1 g, 76.7%) as a white solid. MS (ESI): mass calcd. for C₁₁H₁₂BrN₃O₂S: 328.98, found: 330.0 [M+H]⁺.

### Ethyl 2-(2-((tert-butoxycarbonyl)amino)thiazolo[5,4-b]pyridin-5-yl)-3,3,3-trifluoro-2-hydroxypropanoate

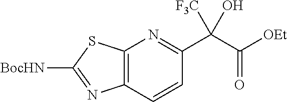 [SMILES: CCOC(=O)C(O)(c1ccc2nc(NC(=O)OC(C)(C)C)sc2n1)C(F)(F)F]

To an ice cold solution of tert-butyl N-(5-bromothiazolo[5,4-b]pyridin-2-yl)carbamate (0.5 g, 1.5 mmol) in THF (5 mL) was added 60% NaH (66.6 mg, 1.67 mmol). After stirring at rt for 30 min, the reaction was cooled down to −78° C., 2.5 M n-BuLi (666 μL, 1.67 mmol) was added dropwise. The reaction was stirred at this temperature for an additional 30 min. Then ethyl 3,3,3-trifluoro-2-oxo-propanoate (201 μL, 1.51 mmol) was added. After stirring for another 3 h, the reaction was quenched with sat. NH₄Cl solution (30 mL) and extracted with EtOAc (25 mL×3). The combined organic layer was washed with brine, dried over Na₂SO₄, filtered, and concentrated. The resulting crude residue was purified by silica gel column chromatography (3-100% EtOAc/PE) to provide the title compound (0.17 g, 26.6%) as a yellow oil. MS (ESI): mass calcd, for C₁₆H₁₈F₃N₃O₅S: 421.09, found: 442.2 [M+H]⁺.

### Ethyl 2-(2-aminothiazolo[5,4-b]pyridin-5-yl)-3,3,3-trifluoro-2-hydroxypropanoate

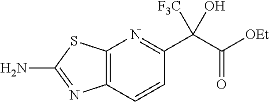 [SMILES: CCOC(=O)C(O)(c1ccc2nc(N)sc2n1)C(F)(F)F]

A solution of ethyl 2-[2-(tert-butoxycarbonylamino)thiazolo[5,4-b]pyridin-5-yl]-3,3,3-trifluoro-2-hydroxy-propanoate (0.17 g, 0.4 mmol) in DCM (5 mL) and TFA (1 mL) was stirred at rt for 2 h. After completion, the reaction was diluted with sat. NaHCO₃ solution (15 mL) and extracted with EtOAc (25 mL×3). The combined organic layer was washed with brine, dried over Na₂SO₄, filtered, and concentrated under reduced pressure. The resulting residue was purified by prep-TLC (100% EtOAc) to afford the title compound as a white solid (68 mg, 52.5%). MS (ESI): mass calcd, for C₁₁H₁₀F₃N₃O₃S: 321.04, found: 322.1 [M+H]⁺.

### N-Ethyl-3,3,3-trifluoro-2-(2-(3-(4-fluorophenyl)ureido)thiazolo[5,4-b]pyridin-5-yl)-2-hydroxypropanamide (147)

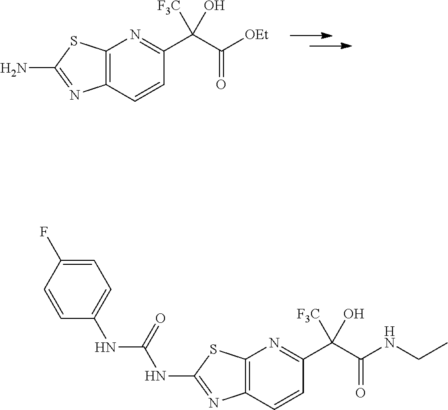 [SMILES: CCOC(=O)C(O)(c1ccc2nc(N)sc2n1)C(F)(F)F ; CCNC(=O)C(O)(c1ccc2nc(NC(=O)Nc3ccc(F)cc3)sc2n1)C(F)(F)F]

The title compound (147) (racemic) was synthesized from ethyl 2-(2-aminothiazolo[5,4-b]pyridin-5-yl)-3,3,3-trifluoro-2-hydroxypropanoate in similar procedures (last 2 steps) as described in Example 61. MS (ESI): mass calcd, for C₁₈H₁₅F₄N₃O₃S: 457.08, found: 458.0 [M+H]⁺. ¹H NMR (400 MHz, DMSO-d6) δ ppm 11.20-11.23 (m, 1H), 9.23 (d, J=8.4 Hz, 1H), 8.41 (t, J=6.0 Hz, 1H), 8.08 (d, J=7.2 Hz, 1H), 7.82 (d, J=8.4 Hz, 1H), 7.48-7.59 (m, 3H), 7.18 (t, J=6.8 Hz, 2H), 3.10-3.17 (m, 2H), 1.00 (t, J=7.2 Hz, 3H).

Example 149 was synthesized in similar procedures as described in Example 38.

### Example 150. 2-(2-(3-(Cyclopropylmethyl)ureido)benzo[d]thiazol-6-yl)-N-ethyl-3,3,3-trifluoro-2-hydroxypropanamide (150)

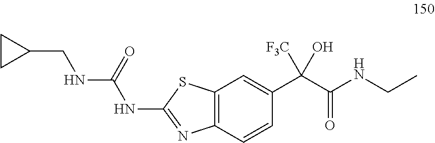 [SMILES: CCNC(=O)C(O)(c1ccc2nc(NC(=O)NCC3CC3)sc2c1)C(F)(F)F]

### Synthetic Scheme:

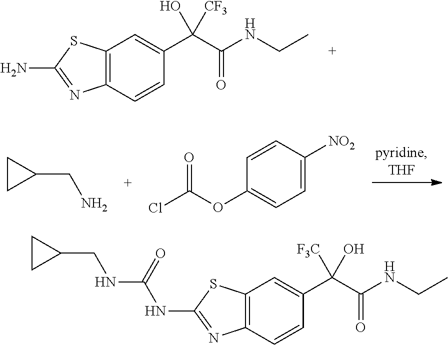

### 2-(2-(3-(Cyclopropylmethyl)ureido)benzo[d]thiazol-6-yl)-N-ethyl-3,3,3-trifluoro-2-hydroxypropanamide (150)

To a solution of ethyl 2-(2-amino-1,3-benzothiazol-6-yl)-N-ethyl-3,3,3-trifluoro-2-hydroxy-propanamide (0.1 g, 0.31 mmol) in THF (3 mL) was added pyridine (0.13 mL, 1.57 mmol) and (4-nitrophenyl) carbonochloridate (126.3 mg, 0.63 mmol). After stirring at 25° C. for 30 min, cyclopropylmethanamine (33.4 mg, 0.47 mmol) was added and the reaction was stirred at 25° C. for an additional 30 min. After completion, the reaction solvent was removed under reduced pressure. The resulting residue was purified by reverse phase HPLC (20-50% ACN in H₂O (10 mM NH₄HCO₃), gradient elution) to provide the title compound (150) (0.03 g, 23%, racemic) as a yellow solid. MS (ESI): mass calcd, for CH₁₉F₁₉N₄O₃S: 416.11, found: 417.0 [M+H]⁺. ¹H NMR (400 MHz, DMSO-d₆) δ ppm 8.23 (t, J=5.6 Hz, 1H), 8.12 (s, 1H), 7.73-7.88 (m, 1H), 7.62 (s, 2H), 6.87 (s, 1H), 6.22-6.30 (m, 1H), 3.08-3.15 (m, 2H), 3.04 (t, J=6.4 Hz, 2H), 0.98 (t, J=6.8 Hz, 4H), 0.41-0.48 (m, 2H), 0.18-0.25 (m, 2H).

Examples 164, 170, 171, 172, 199, 200, 201, and 202 were synthesized in similar procedures as described in Example 150.

Examples 152 was synthesized in similar procedures as described in Example 29.

### Example 153. Ethyl 3,3,3-trifluoro-2-(6-(3-(4-fluorophenyl)ureido)-1-methyl-1H-indol-2-yl)-2-hydroxypropanoate (153)

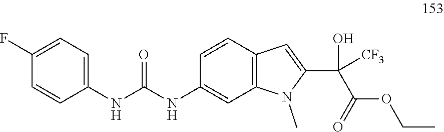 [SMILES: CCOC(=O)C(O)(c1cc2ccc(NC(=O)Nc3ccc(F)cc3)cc2n1C)C(F)(F)F]

### Synthetic Scheme:

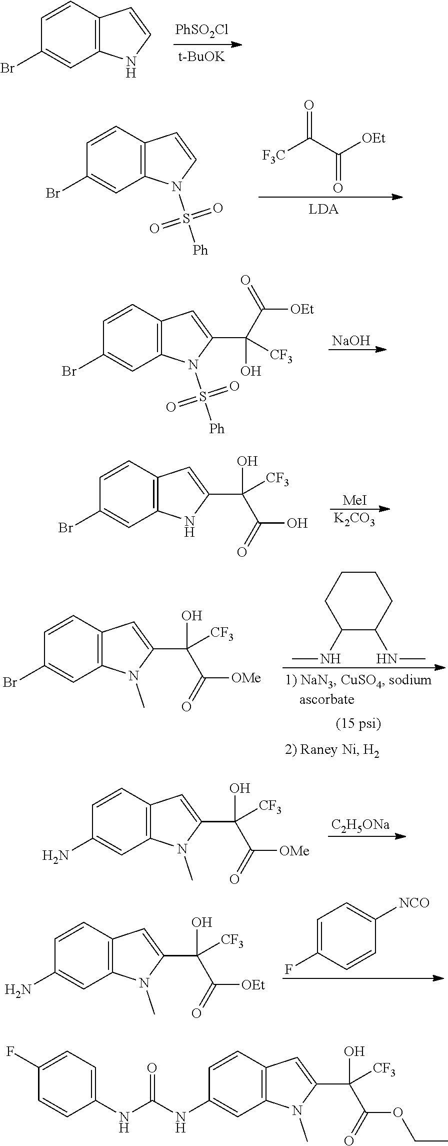

### 6-Bromo-1-(phenylsulfonyl)-1H-indole

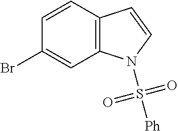 [SMILES: O=S(=O)(c1ccccc1)n1ccc2ccc(Br)cc21]

To an ice-cold solution of 6-bromo-1H-indole (1 g, 5.1 mmol) in THF (10 mL) was added/-BuOK (686.9 mg, 6.12 mmol). After 10 min, benzenesulfonyl chloride (0.78 mL, 6.12 mmol) was added. After stirring at rt for 2 h, the reaction was diluted with H₂O (50 mL) and extracted with EtOAc (50 mL×3). The combined organic layer was washed with brine, dried over Na₂SO₄, filtered, and concentrated under reduced pressure. The resulting residue was purified by silica gel column chromatography (0-20% EtOAc/PE) to afford the title compound (1.5 g, 87.5%) as a pink solid. MS (ESI): mass calcd, for C₁₄H₁₀BrNO₂S: 334.96, found: 336.0 [M+H]⁺.

### Ethyl 2-(6-bromo-1-(phenylsulfonyl)-1H-indol-2-yl)-3,3,3-trifluoro-2-hydroxypropanoate

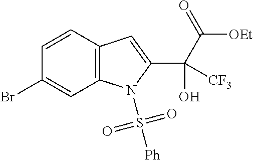 [SMILES: CCOC(=O)C(O)(c1cc2ccc(Br)cc2n1S(=O)(=O)c1ccccc1)C(F)(F)F]

A solution of 1-(benzenesulfonyl)-6-bromo-indole (1.3 g, 3.87 mmol) in THF (13 mL) was degassed and purged with N₂×3. The reaction was cooled down to −78° C., LDA (2.32 mL, 4.64 mmol, 2 M) was added. After stirring for 1 h at the same temperature, a solution of ethyl 3,3,3-trifluoro-2-oxo-propanoate (657.7 mg, 3.87 mmol) in THF (0.51 mL) was added. The resulting solution was stirred at −78° C. for an additional 3 h. After completion, the reaction was quenched with sat. NH₄Cl solution (10 mL)/H₂O (20 mL) and extracted with EtOAc (20 mL×5). The combined organic layer was washed with brine, dried over Na₂SO₄, filtered and concentrated. The crude residue was purified by silica gel column chromatography (0-20% EtOAc/PE) to afford the title compound (1.8 g, 92%) as a yellow solid. MS (ESI): mass calcd, for C₁₉H₁₅BrF₃NO₅S: 504.98, found: 506.1 [M+H]⁺.

### 2-(6-Bromo-1H-indol-2-yl)-3,3,3-trifluoro-2-hydroxypropanoic Acid

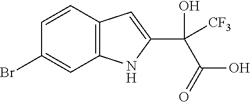 [SMILES: O=C(O)C(O)(c1cc2ccc(Br)cc2[nH]1)C(F)(F)F]

To a solution of ethyl 2-(6-bromo-1-(phenylsulfonyl)-1H-indol-2-yl)-3,3,3-trifluoro-2-hydroxypropanoate (1.2 g, 2.37 mmol) in MeOH (20 mL) and H₂O (2 mL) was added NaOH (237 mg, 5.93 mmol). After heating at 70° C. for 2 h. the reaction was quenched with aqueous citric acid (0.02 mL) and concentrated under reduced pressure to provide the title compound (0.5 g, 62.4%) as a white solid, which was directly used in the next step without further purification. MS (ESI): mass calcd, for C₁₉H₁₅BrF₃NO: 336.96, found: 336.1 [M−H]⁺.

### Methyl 2-(6-bromo-1-methyl-1H-indol-2-yl)-3,3,3-trifluoro-2-hydroxypropanoate

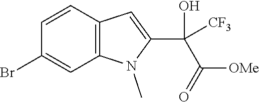 [SMILES: COC(=O)C(O)(c1cc2ccc(Br)cc2n1C)C(F)(F)F]

Iodomethane (828.63 μL, 13.31 mmol) was added to an ice cold suspension of 2-(6-bromo-1H-indol-2-yl)-3,3,3-trifluoro-2-hydroxy-propanoic acid (0.9 g, 2.66 mmol) and K₂CO₃ (294.34 mg, 2.13 mmol) in DMF (10 mL). It was allowed to stir at 40° C. for 12 h. After completion, the reaction mixture was poured into water (30 mL) and stirred for 5 minutes. Then the aqueous layer was extracted with EtOAc (30 mL×3). The combined organic layer was washed with brine, dried over anhydrous Na₂SO₄, filtered, and concentrated in vacuo. The resulting residue was purified by prep-MPLC (0-100% EtOAc/PE) to afford the desired product (0.45 g, 46.2%) as a brown oil. MS (ESI): mass calcd, for C₁₃H₁₁BrF₃NO₃: 364.99, found: 364.0 [M−H]⁺.

### Methyl 2-(6-amino-1-methyl-1H-indol-2-yl)-3,3,3-trifluoro-2-hydroxypropanoate

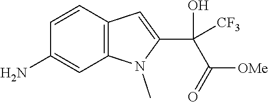 [SMILES: COC(=O)C(O)(c1cc2ccc(N)cc2n1C)C(F)(F)F]

To a suspension of 2-(6-bromo-1-methyl-1H-indol-2-yl)-3,3,3-trifluoro-2-hydroxypropanoate (0.2 g, 1.91 mmol) and NaN₃ (106.5 mg, 1.64 mmol) in EtOH (10 mL) and H₂O) (2 mL) were added CuSO₄ (87.2 mg, 0.55 mmol), N1,N2-dimethylcyclohexane-1,2-diamine (46.6 mg, 0.33 mmol) and sodium ascorbate (216.4 mg, 1.1 mmol) at rt. After heating at 80° C. for 12 h, the reaction was diluted with water (5 mL) and adjusted to pH=9-10 with sat. NaHCO₃ solution. The reaction was extracted with TBME (10 mL×2). The combine red organic layer was washed with bring, dried over Na₂SO₄ and filtered. Raney Ni (0.2 g) was then added to the filtrate. The resulting mixture was purged with H₂×3 and then stirred under H₂ (15 psi) at 50° C. for 3 h. Upon completion, the reaction was filtered through a celite pad, rinsed with MeOH (20 mL) and concentrated. The crude material was purified by prep-TLC (100% EtOAc) to afford the title compound (0.09 g, 54.5%) as a red color oil. MS (ESI): mass calcd, for C₁₃H₁₃F₃N₂O₃: 302.09, found: 301.2 [M−H]⁺.

### Ethyl 2-(6-amino-1-methyl-1H-indol-2-yl)-3,3,3-trifluoro-2-hydroxypropanoate

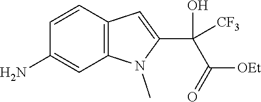 [SMILES: CCOC(=O)C(O)(c1cc2ccc(N)cc2n1C)C(F)(F)F]

To a solution of methyl 2-(6-amino-1-methyl-1H-indol-2-yl)-3,3,3-trifluoro-2-hydroxypropanoate (0.05 g, 0.17 mmol) in EtOAc/EtOH (0.4 mL, 1:1) was added 20% sodium ethoxide (28.1 mg, 83 μmol) and sodium tert-butoxide (15.9 mg, 0.17 mmol). After stirring at rt for 2 h, the reaction was quenched with 1 N HCl (0.1 mL) and H₂O (3 mL) at 0° C., and then extracted with EtOAc (3 mL×3). The combined organic layer was washed with brine, dried over Na₂SO₄, filtered, and concentrated to afford the title compound (0.04 g, 76.5%) as a black solid, which was directly used in the next step without further purification. MS (ESI): mass calcd, for C₁₉H₁₅BrF₃NO₅S: 316.10, found: 317.2 [M+H]⁺.

### Ethyl 3,3,3-trifluoro-2-(6-(3-(4-fluorophenyl)ureido)-1-methyl-1H-indol-2-yl)-2-hydroxypropanoate (153)

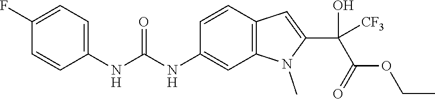 [SMILES: CCOC(=O)C(O)(c1cc2ccc(NC(=O)Nc3ccc(F)cc3)cc2n1C)C(F)(F)F]

The title compound (153) was synthesized from ethyl 3,3,3-trifluoro-2-hydroxy-2-(1-methyl-2-(3-phenylureido)-1H-benzo[d]imidazol-5-yl)propanoate in a similar step as described in Example 1. MS (ESI): mass calcd, for C₂₁H₁₉F₄N₃O₄: 453.13, found: 454.1 [M+H]⁺. ¹H NMR (400 MHz, DMSO-d6) δ ppm 11.21 (s, 1H), 8.64 (d, J=1.6 Hz, 2H), 7.81 (s, 1H), 7.45-7.49 (m, 3H), 7.12 (t, J=8.8 Hz, 2H), 6.95 (d, J=2.0 Hz, 1H), 6.60 (s, 1H), 4.38-4.44 (m, 2H), 3.36 (s, 3H), 1.31 (t, J=7.2 Hz, 3H).

Example 154 was synthesized in similar procedures as described in Example 36.

Examples 156 was synthesized in similar procedures as described in Example 27

Examples 159 was synthesized in similar procedures as described in Example 38.

Examples 160 and 161 were synthesized from Example 156 by chiral SFC separation.

Example 162 was the 2^{nd} eluting peak from step 1 SFC separation in Example 38.

### Example 165. N-Ethyl-3,3,3-trifluoro-2-hydroxy-2-(2-(2-(pyrrolidin-1-yl)acetamido)benzo[d]thiazol-6-yl)propanamide (165)

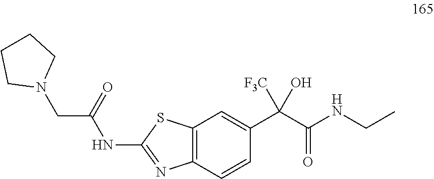 [SMILES: CCNC(=O)C(O)(c1ccc2nc(NC(=O)CN3CCCC3)sc2c1)C(F)(F)F]

### Synthetic Scheme:

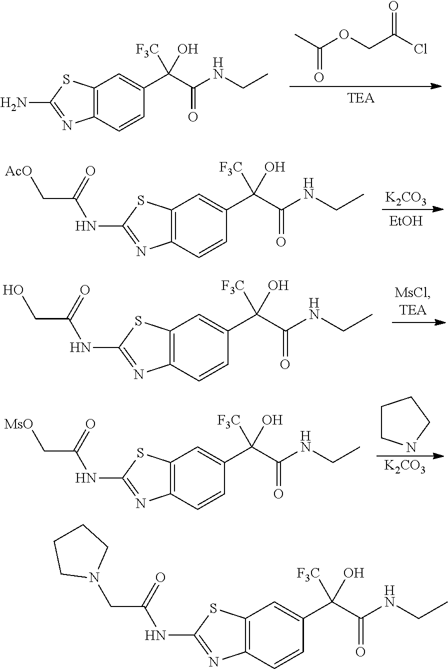

### 2-((6-(3-(Ethylamino)-1,1,1-trifluoro-2-hydroxy-3-oxopropan-2-yl)benzo[d]thiazol-2-yl)amino)-2-oxoethyl acetate

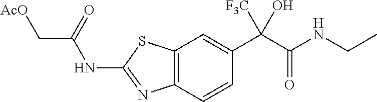 [SMILES: CCNC(=O)C(O)(c1ccc2nc(NC(=O)COC(C)=O)sc2c1)C(F)(F)F]

TEA (0.45 mL, 3.19 mmol) and 2-chloro-2-oxoethyl acetate (82 μL, 0.77 mmol) were added to a solution of 2-(2-aminobenzo[d]thiazol-6-yl)-N-ethyl-3,3,3-trifluoro-2-hydroxypropanamide (204 mg, 0.64 mmol) in THF (3.2 mL) at rt. After 2 h, additional 2-chloro-2-oxoethyl acetate (0.25 mL, 2.34 mmol) and TEA (1 mL, 7.17 mmol) were added. After 1 h, the reaction was diluted with sat. NH₄Cl and extracted with EtOAc (20 mL×3). The combined organic layer was washed with brine, dried over Na₂SO₄, filtered, and concentrated to afford the crude compound as a yellow oil (268 mg), which was used in the next step directly without further purification. MS (ESI): mass calcd, for C₁₆H₁₆F₃N₃O₅S: 419.08, found: 419.85 [M+H]⁺.

### N-Ethyl-3,3,3-trifluoro-2-hydroxy-2-(2-(2-hydroxyacetamido)benzo[d]thiazol-6-yl)propanamide

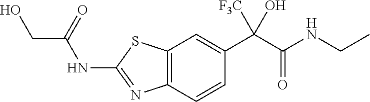 [SMILES: CCNC(=O)C(O)(c1ccc2nc(NC(=O)CO)sc2c1)C(F)(F)F]

To a solution of 2-((6-(3-(ethylamino)-1,1,1-trifluoro-2-hydroxy-3-oxopropan-2-yl)benzo[d]thiazol-2-yl)amino)-2-oxoethyl acetate (268 mg, 0.64 mmol) in EtOH (6.4 mL) was added K₂CO₃ (44.2 mg, 0.32 mmol). After stirring overnight at rt. the solvent was removed under reduced pressure. The reaction was diluted with water and extracted with 30% IP A/chloroform (×3). The combined organic layer was dried over Na₂SO₄, filtered, and concentrated. The crude residue was purified by silica gel column chromatography (30-70% EtOAc/Hex) to afford the title compound (111 mg, 46%) as a colorless oil. MS (ESI): mass calcd, for C₁₄H₁₄F₃N₃O₄S: 377.07. found: 377.85 [M+H]⁺.

### 2-((6-(3-(Ethylamino)-1,1,1-trifluoro-2-hydroxy-3-oxopropan-2-yl)benzo[d]thiazol-2-yl)amino)-2-oxoethyl methanesulfonate

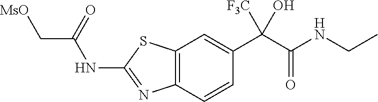 [SMILES: CCNC(=O)C(O)(c1ccc2nc(NC(=O)COS(C)(=O)=O)sc2c1)C(F)(F)F]

To an ice-cold solution of N-ethyl-3,3,3-trifluoro-2-hydroxy-2-(2-(2-hydroxyacetamido)benzo[d]thiazol-6-yl)propenamide (111 mg, 0.29 mmol) in THF (2.9 mL) was added methanesulfonyl chloride (30 μL, 0.38 mmol) and TEA (82 μL, 0.59 mmol). After stirring at 0° C. for 10 min, the reaction was quenched with water and extracted with DCM×3. The combined organic layer was dried with Na₂SO₄, filtered, and concentrated under reduced pressure to afford the title compound as a yellow oil (134 mg), which was directly used in the next step without further purification. MS (ESI): mass calcd, for C₁₅H₁₆F₃N₃O₆S₂: 455.04, found: 455.85 [M+H]⁺.

### N-Ethyl-3,3,3-trifluoro-2-hydroxy-2-(2-(2-(pyrrolidin-1-yl)acetamido)benzo[d]thiazol-6-yl)propenamide (165)

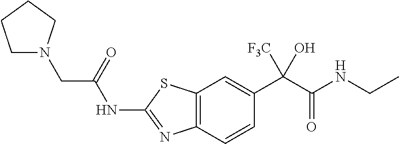 [SMILES: CCNC(=O)C(O)(c1ccc2nc(NC(=O)CN3CCCC3)sc2c1)C(F)(F)F]

To a solution of 2-((6-(3-(ethylamino)-1,1,1-trifluoro-2-hydroxy-3-oxopropan-2-yl)benzo[d]thiazol-2-yl)amino)-2-oxoethyl methanesulfonate (30 mg, 0.067 mmol) in DMF (0.67 mL) was added pyrrolidine (7 μL, 0.087 mmol) and K₂CO₃ (27.7 mg, 0.067 mmol). After stirring at rt for 1 h, the reaction was diluted with EtOAc and washed with water. The organic layer was dried, concentrated and purified by reverse phase HPLC (10-50% ACN/H₂O, with 0.1% TFA as a modifier) to provide the title compound (165) (2.2 mg, 7.5%, racemic) as a white solid. MS (ESI): mass calcd, for C₁₈H₂₁F₃N₄O₃S: 430.13, found: 430.95 [M+H]⁺. ¹H NMR (400 MHz, DMSO-d₆) δ ppm 10.18 (br s, 1H), 8.29 (s, 1H), 8.26 (t, J=5.8 Hz, 1H), 7.87 (s, 1H), 7.78 (q, J=13.0 Hz, 2H), 4.42 (s, 2H), 3.63 (br s, 1H), 3.09-3.21 (m, 3H), 2.93 (dq, J=13.0, 6.7 Hz, 2H), 1.91-2.03 (m, 4H), 0.99 (t, J=7.1 Hz, 3H).

Examples 141, 151, 166, 167, and 168 were synthesized in similar procedures as described in Example 165.

Examples 173 was synthesized in similar procedures as described in Example 27.

Example 174 was synthesized in similar procedures as described in Example 29.

### Example 175. (S)-(3,3,3-Trifluoro-2-(2-(3-(4-fluorophenyl)ureido)benzo[d]thiazol-6-yl)-2-hydroxypropanoyl)glycine (175)

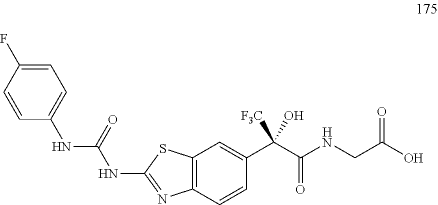 [SMILES: O=C(O)CNC(=O)[C@@](O)(c1ccc2nc(NC(=O)Nc3ccc(F)cc3)sc2c1)C(F)(F)F]

### Synthetic Scheme:

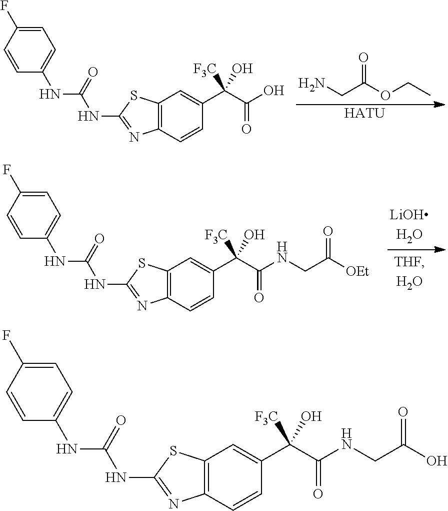

### Ethyl (S)-(3,3,3-trifluoro-2-(2-(3-(4-fluorophenyl)ureido)benzo[d]thiazol-6-yl)-2-hydroxypropanoyl)glycinate

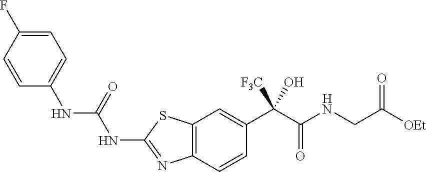 [SMILES: CCOC(=O)CNC(=O)[C@@](O)(c1ccc2nc(NC(=O)Nc3ccc(F)cc3)sc2c1)C(F)(F)F]

The title compound was synthesized from ethyl 3,3,3-trifluoro-2-hydroxy-2-(2-(3-phenylureido)quinolin-6-yl)propanoate in similar procedures as described in Example 38. MS (ESI): mass calcd, for C₂₁H₁₈F₄N₄O₅S: 514.09, found: 515.1 [M+H]⁺.

### (S)-(3,3,3-Trifluoro-2-(2-(3-(4-fluorophenyl)ureido)benzo[d]thiazol-6-yl)-2-hydroxypropanoyl)glycine (175)

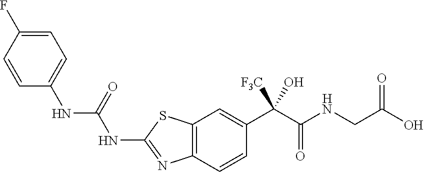 [SMILES: O=C(O)CNC(=O)[C@@](O)(c1ccc2nc(NC(=O)Nc3ccc(F)cc3)sc2c1)C(F)(F)F]

A suspension of ethyl (S)-(3,3,3-trifluoro-2-(2-(3-(4-fluorophenyl)ureido)benzo[d]thiazol-6-yl)-2-hydroxypropanoyl)glycinate (20 mg, 0.039 mmol) and LiOH·H₂O (8.2 mg, 0.19 mmol) in THF (2 mL) and H₂O (0.1 mL) was stirred at rt for 2 h. The reaction was adjusted to pH=2 with 1 N HCl and further diluted with H₂O (5 mL). The aqueous layer was extracted with EtOAc (5 mL×3). The combined organic layer was dried over Na₂SO₄, filtered, and concentrated. The crude residue was purified by reverse phase HPLC (1-45% ACN in H₂O (10 mM NH₄HCO₂), gradient elution) to provide the title product (175) (13 mg, 34.4%) as a white solid. MS (ESI): mass calcd, for C₁₅H₁₄F₄N₄O₅S: 486.06, found: 487.0 [M+H]⁺. ¹H NMR (400 MHz, DMSO-d₆) δ ppm 8.38 (t, J=5.6 Hz, 1H), 8.13 (s, 1H), 7.97 (s, 2H), 7.72 (s, 1H), 7.59-7.63 (m, 2H), 7.15 (t, J=8.8 Hz, 2H), 3.85-3.91 (m, 1H), 3.61-3.66 (m, 1H).

Examples 176, 177, 178, 179, 189, 194, 195, 196, 197, 198, 206, 207, 208, 210, and 211 were synthesized in similar procedures as described in Example 175.

Examples 180, 182, 183 were synthesized in similar procedures as described in Example 38.

### Example 184. 3,3,3-Trifluoro-2-(2-(3-(4-fluorophenyl)ureido)-6,7-dihydrothiazolo[5,4-c]pyridin-5 (4H)-yl)propanoic Acid (184)

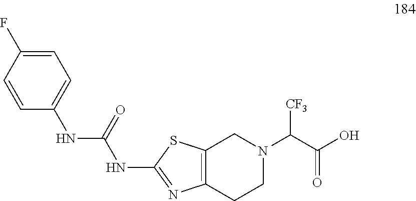 [SMILES: O=C(Nc1ccc(F)cc1)Nc1nc2c(s1)CN(C(C(=O)O)C(F)(F)F)CC2]

### Synthetic Scheme:

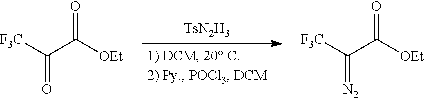

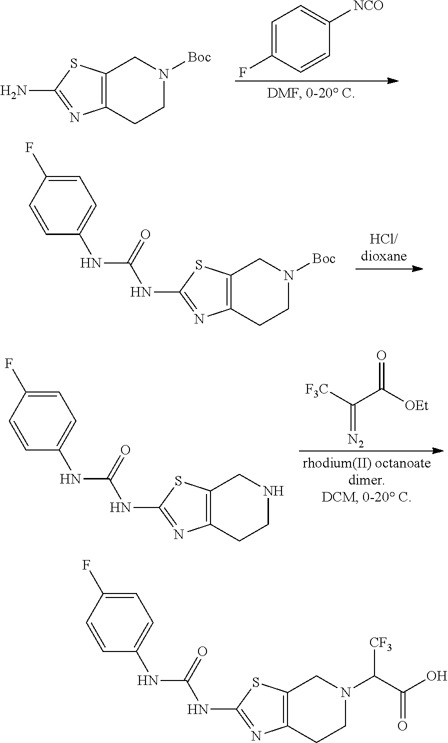

### Ethyl 2-diazo-3,3,3-trifluoropropanoate

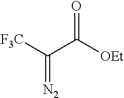

To a solution of ethyl 3,3,3-trifluoro-2-oxo-propanoate (3.9 mL, 29.4 mmol) in DCM (50 mL) was added 4-methylbenzenesulfonohydrazide (6.57 g, 35.3 mmol). After heating at 60° C. equipped with a reflux condenser for 18 b, pyridine (13 mL, 161.7 mmol) was added, followed by dropwise addition of POCl (2.74 mL, 29.4 mmol) via a gas-tight syringe. The reaction was continued at this temperature for an additional 30 min before cooling to rt. TEA (4.91 mL, 35.3 mmol) was added dropwise. Then the reaction was heated at 60° C. and stirred for another 30 min before cooling to rt and quenching with H₂O (50 mL). The aqueous layer was extracted with DCM (50 mL×3). The combined organic layer was washed with 1 N HCl (50 mL), NaHCO₃ (50 mL), brine (10 mL), dried over Na₂SO₄, filtered, and concentrated under reduced pressure. The crude product was purified by vacuum distillation (70° C., 10 KPa/water pump) to afford the desired product (3 g, 56%) as a yellow oil. ¹H NMR (400 MHz, CDCl₃) δ ppm 4.32 (q, J=7.2 Hz, 2H), 1.32 (t, J=7.2 Hz, 3H).

### tert-Butyl 2-(3-(4-fluorophenyl)ureido)-6,7-dihydrothiazolo[5,4-c]pyridine-5(4H)-carboxylate

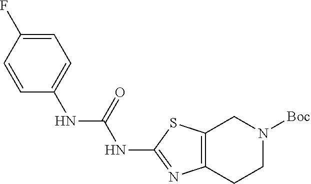 [SMILES: CC(C)(C)OC(=O)N1CCc2nc(NC(=O)Nc3ccc(F)cc3)sc2C1]

To an ice-cold solution of tert-butyl 2-amino-6,7-dihydro-4H-thiazolo[5,4-c]pyridine-5-carboxylate (1 g, 3.9 mmol) in DMF (10 mL) was added 1-fluoro-4-isocyanato-benzene (483 μL) dropwise. After stirring at rt for 2 h, the reaction was quenched with H₂O (100 mL). The resulting precipitate was filtered and dried to obtain the title compound (1.2 g, 71.4%) as a white solid. MS (ESI): mass calcd, for C₁₈H₂₁FN₄O₃S: 392.13, found: 393.2 [M+H]⁺.

### 1-(4-Fluorophenyl)-3-(4,5,6,7-tetrahydrothiazolo[5,4-c]pyridin-2-yl)urea

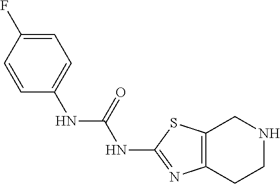 [SMILES: O=C(Nc1ccc(F)cc1)Nc1nc2c(s1)CNCC2]

tert-Butyl 2-[(4-fluorophenyl) carbamoylamino]-6,7-dihydro-4H-thiazolo[5,4-c]pyridine-5-carboxylate (500 mg, 1.27 mmol) was dissolved in dioxane (2 mL) and added to 4 M HCl in dioxane (4 mL). After stirring at rt for 8 h, the reaction was quenched with sat. NaHCO₃ (6 mL). The resulting precipitate was filtered and dried to afford the title compound (357 mg, 96%) as a white solid, which was directly used in the next step without further purification. MS (ESI): mass calcd, for C₁₃H₁₃FN₄OS: 292.08, found: 293.1 [M+H]⁺.

### 3,3,3-Trifluoro-2-(2-(3-(4-fluorophenyl)ureido)-6,7-dihydrothiazolo[5,4-c]pyridin-5 (4H)-yl)propanoic Acid (184)

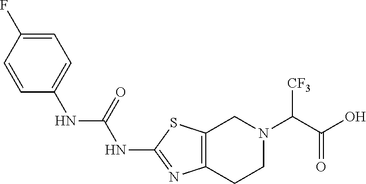 [SMILES: O=C(Nc1ccc(F)cc1)Nc1nc2c(s1)CN(C(C(=O)O)C(F)(F)F)CC2]

To a solution of 1-(4-fluorophenyl)-3-(4,5,6,7-tetrahydrothiazolo[5,4-c]pyridin-2-yl)urea (185 mg, 0.63 mmol) in DCM (3 mL) was added di(octanoyloxy)rhodium (4.93 mg, 12.7 μmol) and ethyl 2-diazo-3,3,3-trifluoro-propanoate (345.7 mg, 1.9 mmol). It was stirred at 40° C. for 18 h. After completion, the reaction mixture was cooled to rt and quenched with H₂O (3 mL). The aqueous layer was extracted with EtOAc (3 mL×2). The combined organic layer was washed with brine, dried over Na₂SO₄, filtered, and concentrated under reduced pressure. The resulting residue was purified by reverse phase HPLC (25-55% ACN in H₂O (10 mM NH₄HCO₃), gradient elution) to provide the title compound (13 mg, 4.9%) as a white solid. MS (ESI): mass calcd, for C₁₆H₁₄F₄N₄O₃S: 418.07, found: 419.0 [M+H]⁺. ¹H NMR (400 MHz, DMSO-d6) δ ppm 10.35-10.46 (m, 1H), 9.03 (t, J=6.0 Hz, 1H), 7.48 (t, J=7.2 Hz, 2H), 7.11-7.18 (m, 2H), 6.78-6.94 (m, 1H), 5.20-5.24 (m, 1H), 4.51-4.76 (m, 2H), 3.80-3.88 (m, 2H), 2.71 (s, 1H), 2.61-2.67 (m, 1H).

Examples 187 and 188 were synthesized in similar procedures as described in Example 27.

Example 190 was synthesized in similar procedures as described in Example 38.

### Example 191 and 192. (R)—N-Ethyl-3,3-difluoro-2-(2-(3-(4-fluorophenyl)ureido)benzo[d]thiazol-6-yl)-2-hydroxypropanamide (191) & (S)—N-Ethyl-3,3-difluoro-2-(2-(3-(4-fluorophenyl)ureido)benzo[d]thiazol-6-yl)-2-hydroxypropanamide (192)

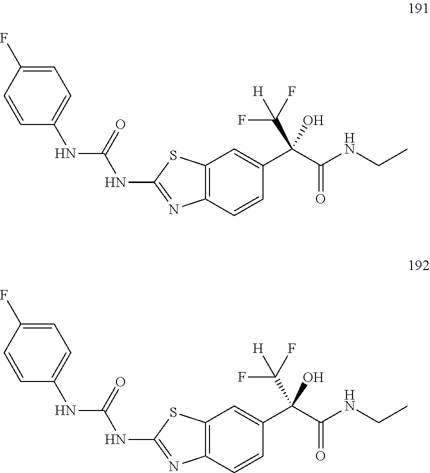 [SMILES: CCNC(=O)[C@@](O)(c1ccc2nc(NC(=O)Nc3ccc(F)cc3)sc2c1)C(F)F ; CCNC(=O)[C@](O)(c1ccc2nc(NC(=O)Nc3ccc(F)cc3)sc2c1)C(F)F]

### Synthetic Scheme:

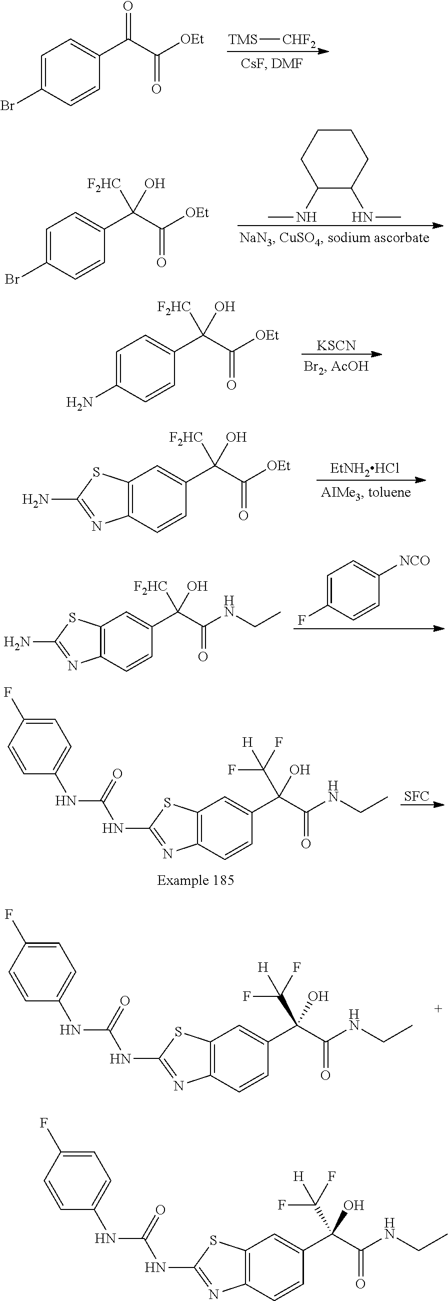

### Ethyl 2-(4-bromophenyl)-3,3-difluoro-2-hydroxypropanoate

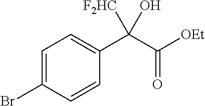 [SMILES: CCOC(=O)C(O)(c1ccc(Br)cc1)C(F)F]

To a solution of ethyl 2-(4-bromophenyl)-2-oxo-acetate (3 g, 11.67 mmol) in DMF (30 mL) was added CsF (886.3 mg, 5.83 mmol) and difluoromethyl(trimethyl) silane (2.90 g. 23.34 mmol). After stirring at rt for 12 h, the reaction was filtered through a celite pad and rinsed with EtOAc (30 mL). The filtrate was concentrated under reduced pressure and purified by reverse phase HPLC (40-70% ACN in H₂O (10 mM NH₄HCO₃), gradient elution) to provide the title compound (0.8 g, 22.2%) as a white solid. MS (ESI): mass calcd. for C₁₉H₁₄F₄N₆O₂S: 466.08, found: 467.0 [M+H]⁺.

### N-Ethyl-3,3-difluoro-2-(2-(3-(4-fluorophenyl)ureido)benzo[d]thiazol-6-yl)-2-hydroxypropanamide (Example 185)

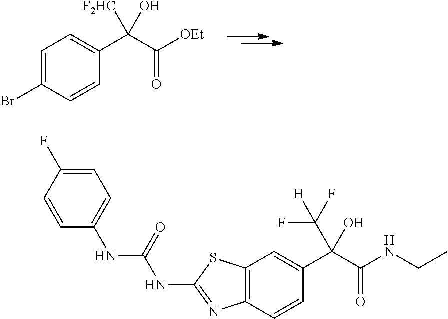 [SMILES: CCOC(=O)C(O)(c1ccc(Br)cc1)C(F)F ; CCNC(=O)C(O)(c1ccc2nc(NC(=O)Nc3ccc(F)cc3)sc2c1)C(F)F]

The title compound (Example 185) was synthesized from ethyl 2-(4-bromophenyl)-3,3-difluoro-2-hydroxypropanoate in similar procedures as described in Example 61. MS (ESI): mass calcd, for C₁₉H₁₇F₃N₄O₃S: 438.10, found: 439.1 [M+H]⁺. ¹H NMR (400 MHz, DMSO-d6) δ ppm 10.79-10.87 (m, 1H), 9.18-9.22 (m, 1H), 8.12 (t, J=6.0 Hz, 2H), 7.53-7.67 (m, 4H), 7.17 (t, J=8.8 Hz, 2H), 7.00 (s, 1H), 6.77 (t, J=54.0 Hz, 1H), 3.00-3.13 (m, 2H), 0.96 (t, J=7.2 Hz, 3H)

### (R)—N-Ethyl-3,3-difluoro-2-(2-(3-(4-fluorophenyl)ureido)benzo[d]thiazol-6-yl)-2-hydroxypropanamide (191) & (S)—N-Ethyl-3,3-difluoro-2-(2-(3-(4-fluorophenyl)ureido)benzo[d]thiazol-6-yl-2-hydroxypropanamide (192)

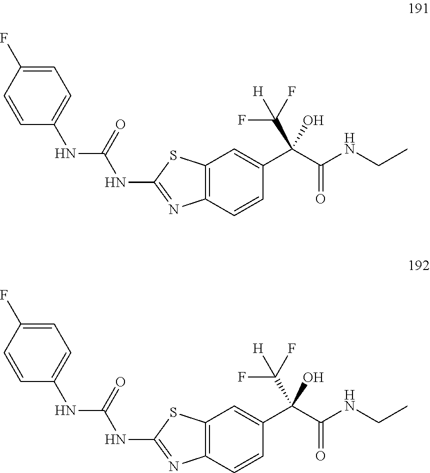 [SMILES: CCNC(=O)[C@@](O)(c1ccc2nc(NC(=O)Nc3ccc(F)cc3)sc2c1)C(F)F ; CCNC(=O)[C@](O)(c1ccc2nc(NC(=O)Nc3ccc(F)cc3)sc2c1)C(F)F]

N-ethyl-3,3-difluoro-2-(2-(3-(4-fluorophenyl)ureido)benzo[d]thiazol-6-yl)-2-hydroxypropanamide (Example 185) was separated by chiral SFC separation (column: REGIS (s,s) WHELK-O1 (250 mm*30 mm, 5 μm); mobile phase: 50% MeOH with 0.1% NH₃H₂O) to afford the title compound (191) (17 mg, 34%) as a white solid. MS (ESI): mass calcd, for C₁₉H₁₇F₄N₄O₃S: 438.1, found: 439.1 [M+H]⁺. ¹H NMR (400 MHz, DMSO-d₆) δ ppm 10.93-11.04 (m, 1H), 9.29 (s, 1H), 8.11 (t, J=6.4 Hz, 2H), 7.62 (s, 2H), 7.54 (s, 2H), 7.17 (t, J=8.8 Hz, 2H), 7.00 (s, 1H), 6.77 (t, J=54.4 Hz, 1H), 3.03-3.10 (m, 2H), 0.96 (t, J=7.2 Hz, 3H); and the title compound (192) (15 mg, 30%) as a white solid. MS (ESI): mass calcd, for C₁₉H₁₇F₄N₄O₃S: 438.1, found: 439.1 [M+H]⁺. ¹H NMR (400 MHz, DMSO-d6) δ ppm 10.92-11.01 (m, 1H), 9.31 (s, 1H), 8.12 (t, J=6.4 Hz, 2H), 7.62 (s, 2H), 7.52-7.56 (m, 2H), 7.17 (t, J=8.8 Hz, 2H), 7.00 (s, 1H), 6.77 (t, J=54.4 Hz, 1H), 3.00-3.12 (m, 2H), 0.96 (t, J=7.2 Hz, 3H).

Example 193 were synthesized in similar procedures as described in Example 38.

### Example 203. 2-Amino-N-ethyl-3,3,3-trifluoro-2-(2-(3-(4-fluorophenyl)ureido)benzo[d]thiazol-6-yl)propenamide (203)

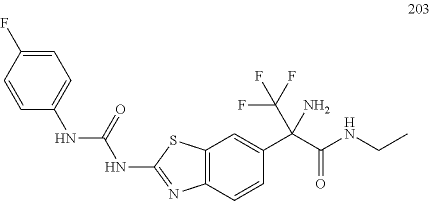 [SMILES: CCNC(=O)C(N)(c1ccc2nc(NC(=O)Nc3ccc(F)cc3)sc2c1)C(F)(F)F]

### Synthetic Scheme:

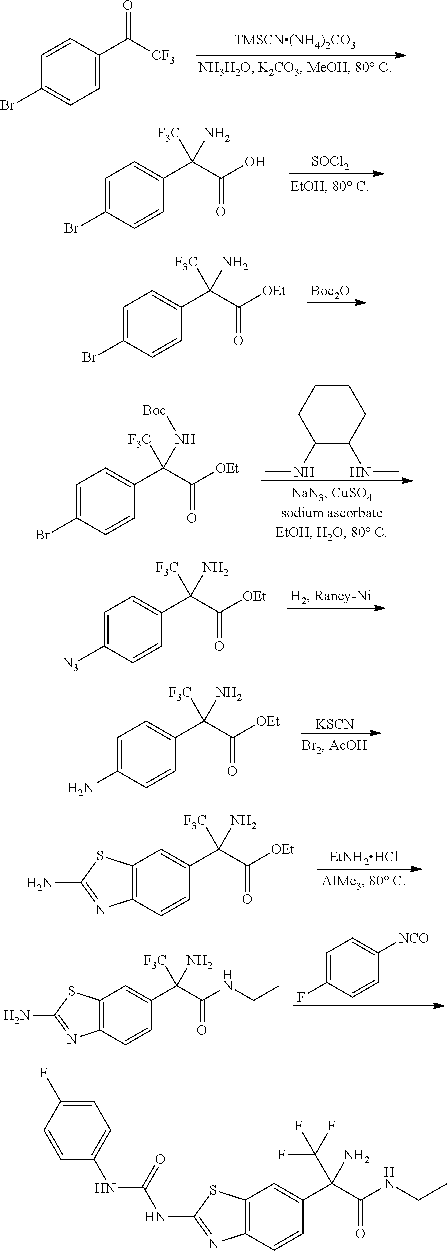

### 2-Amino-2-(4-bromophenyl)-3,3,3-trifluoropropanoic Acid

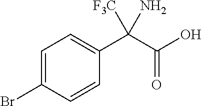 [SMILES: NC(C(=O)O)(c1ccc(Br)cc1)C(F)(F)F]

To a solution of 1-(4-bromophenyl)-2,2,2-trifluoro-ethanone (12 mL, 79.1 mmol) in MeOH (200 mL), H₂O (200 mL) and NH₃·H₂O (200 mL) was added TMSCN (39.6 mL, 316.2 mmol), (NH₄)₂CO₃ (67.5 mL, 632.4 mmol) and K₂CO₃ (65.6 g, 474.3 mmol). After heating at 80° C. for 12 h, the reaction solvent was removed under reduced pressure. The resulting residue was dissolved in H₂O (200 mL) and extracted with EtOAc (500 mL×2). The combined organic layer was dried over Na₂SO₄, filtered, concentrated, and purified by silica gel column chromatography (10-100% EtOAc/PE) to afford the title compound (6.98 g, 29.6%) as a brown oil. ¹H NMR (400 MHz, DMSO-d₆) δ ppm 7.76 (s, 1H), 7.69 (br s, 1H), 7.63 (m, 4H), 7.60 (br s, 1H).

### Ethyl 2-amino-2-(4-bromophenyl)-3,3,3-trifluoropropanoate

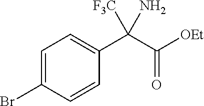 [SMILES: CCOC(=O)C(N)(c1ccc(Br)cc1)C(F)(F)F]

Conc. H₂SO₄ (4.29 mL, 80.5 mmol) was added to an ice-cold solution of 2-amino-2-(4-bromophenyl)-3,3,3-trifluoro-propanoic acid (4.8 g, 16.1 mmol) in EtOH (48 mL). After beating at 80° C. for 12 h, the reaction was cooled down to rt, quenched with careful addition of sat. NaHCO₃ solution (500 mL) and extracted with EtOAc (100 mL×3). The combined organic layer was washed with brine, dried over Na₂SO₄, filtered, concentrated and purified by silica gel column chromatography (0-20% EtOAc/PE) to provide the title compound (3.54 g, 67.4%) as a colorless oil, ¹H NMR (400 MHz, CDCl₃) δ ppm 7.97 (s, 1H), 7.65-7.69 (m, 2H), 7.52 (d, J=8.4 Hz, 2H), 4.24-4.30 (m, 2H), 1.21 (t, J=7.2 Hz, 3H).

### Ethyl 2-(4-bromophenyl)-2-((tert-butoxycarbonyl)amino)-3,3,3-trifluoropropanoate

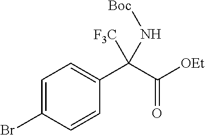 [SMILES: CCOC(=O)C(NC(=O)OC(C)(C)C)(c1ccc(Br)cc1)C(F)(F)F]

To an ice-cold solution of ethyl 2-amino-2-(4-bromophenyl)-3,3,3-trifluoro-propanoate (4.2 g, 12.88 mmol) in DCM (50 mL), TEA (5.38 mL, 38.64 mmol) and Boc₂O (4.44 mL, 19.32 mmol) were added. The reaction was heated to 90° C. for 12 h (DCM was all evaporated). After completion, the reaction was diluted with H₂O (25 mL) and extracted with DCM (50 mL×3). The combined organic layer was washed with brine, dried over Na₂SO₄, filtered, concentrated and purified by silica gel column chromatography (0-5% EtOAc/PE) to afford the title compound (5.4 g, 98.4%) as a white solid. ¹H NMR (400 MHz, CDCl₃) δ ppm 7.71-7.74 (m, 2H), 7.53 (d, J=8.8 Hz, 2H), 4.29-4.35 (m, 2H), 1.45 (s, 9H), 1.20 (t, J=7.2 Hz, 3H).

### 2-Amino-N-ethyl-3,3,3-trifluoro-2-(2-(3-(4-fluorophenyl)ureido)benzo[d]thiazol-6-yl)propenamide (203)

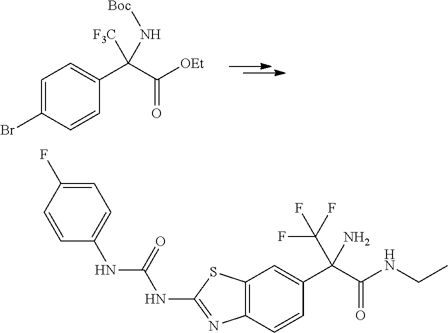

The title compound (203) was prepared from ethyl 2-(4-bromophenyl)-2-((tert-butoxycarbonyl)amino)-3,3,3-trifluoropropanoate following Example 72, steps 3 to 5, and Example 61, step 4 and 5 procedures. MS (ESI): mass calcd, for C₁₉H₁₇F₄N₅O₂S: 455.10, found: 456.10 [M+H]⁺. ¹H NMR (400 MHz, DMSO-d₆) δ ppm 10.88 (br s, 1H). 9.20 (s, 1H). 8.67 (s, 1H), 8.16 (s, 1H), 8.00 (s, 1H), 7.62 (s, 1H), 7.54 (s, 2H), 7.43-7.46 (m, 1H), 7.17 (t, J=8.8 Hz, 2H), 7.13 (t, J=8.8 Hz, 1H), 3.07-3.14 (m, 2H), 0.97 (t, J=7.2 Hz, 3H).

Examples 204 and 205 were synthesized from Example 188 by chiral SFC separation.

Example 209 was synthesized in similar procedures as described in Example 27.

### Example 212. N-Ethyl-3,3,3-trifluoro-2-hydroxy-2-(2-(3-(4-(trifluoromethoxy)phenyl)ureido)benzo[d]thiazol-6-yl)propanamide (212)

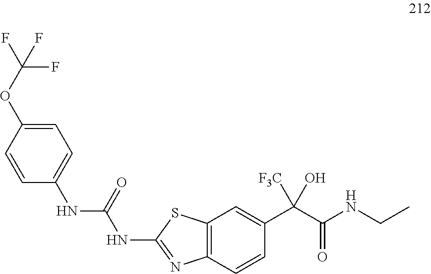 [SMILES: CCNC(=O)C(O)(c1ccc2nc(NC(=O)Nc3ccc(OC(F)(F)F)cc3)sc2c1)C(F)(F)F]

### Synthetic Scheme:

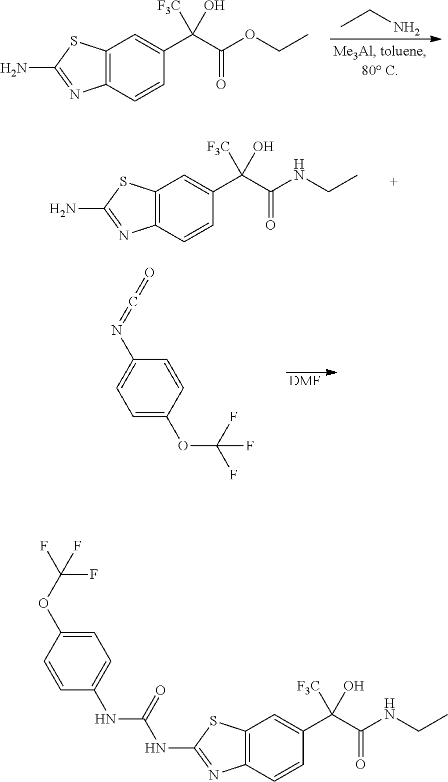

### 2-(2-Aminobenzo[d]thiazol-6-yl)-N-ethyl-3,3,3-trifluoro-2-hydroxypropanamide

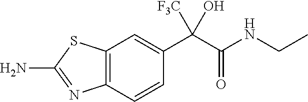 [SMILES: CCNC(=O)C(O)(c1ccc2nc(N)sc2c1)C(F)(F)F]

To a solution of ethyl amine hydrochloride (153 mg, 1.87 mmol) in toluene (6 mL) at 0° C. was added trimethylaluminum (2 M in toluene) (0.94 mL, 1.87 mmol) slowly and the mixture was stirred at 0° C. for 30 min then at rt for 1 h. Ethyl 2-(2-aminobenzo[d]thiazol-6-yl)-3,3,3-trifluoro-2-hydroxypropanoate (200 mg, 0.62 mmol, see Example 6 for synthesis) in toluene (6 mL) was added and the mixture was heated at 80° C. for 2 h. The reaction was quenched with 5% aqueous HCl solution, then the pH was adjusted to 8 with 1 M aqueous NaOH solution. The aqueous phase was extracted with EtOAc (70 ml×2). The combined organic layer was washed with brine, dried over Na₂SO₄, filtered, and concentrated. The crude was purified by silica gel column chromatography (25-100% EtOAc/Heptanes) to give the title compound (120 mg, 60%). MS (ESI): mass calcd, for C₁₂H₁₂F₃N₃O₂S: 319.06, found: 320.1 [M+H]⁺.

### N-Ethyl-3,3,3-trifluoro-2-hydroxy-2-(2-(3-(4-(trifluoromethoxy)phenyl)ureido)benzo[d]thiazol-6-yl)propanamide (212)

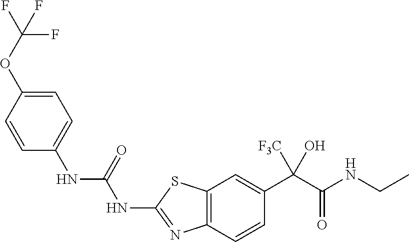 [SMILES: CCNC(=O)C(O)(c1ccc2nc(NC(=O)Nc3ccc(OC(F)(F)F)cc3)sc2c1)C(F)(F)F]

To a solution of 2-(2-aminobenzo[d]thiazol-6-yl)-N-ethyl-3,3,3-trifluoro-2-hydroxypropanamide (15 mg, 47 μmol) in DMF (0.75 mL) was added 4-(trifluoromethoxy)phenyl isocyanate (19 mg, 94 μmol). The reaction was stirred at rt for 30 min and then was directly purified by reverse phase HPLC (C18, 0-100% MeCN/ammonium formate 10 mM buffer) to give the title compound (212) (9.2 mg, 37%, racemic) as a white solid. MS (ESI): mass calcd, for C₂₀H₁₆F₆N₄O₄S: 522.08, found: 523.2 [M+H]⁺. ¹H NMR (400 MHz, DMSO-d6) δ ppm 9.52 (s, 1H), 8.21 (t, J=5.6 Hz, 1H), 8.15 (s, 1H), 7.77 (s, 1H), 7.61-7.70 (m, 4H), 7.33 (d, J=8.7 Hz, 2H), 3.07-3.17 (m, 2H), 0.99 (t, J=7.1 Hz, 3H).

Example 213, Example 214, Example 215, and Example 216 were synthesized from 2-(2-aminobenzo[d]thiazol-6-yl)-N-ethyl-3,3,3-trifluoro-2-hydroxypropanamide following the last step of Example 212, using their respective isocyanates. All are racemic.

### Example 217. 2-(2-((4,5-Dimethylpyrimidin-2-yl)amino)benzo[d]thiazol-6-yl)-N-ethyl-3,3,3-trifluoro-2-hydroxypropanamide (217)

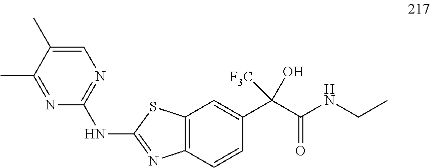 [SMILES: CCNC(=O)C(O)(c1ccc2nc(Nc3ncc(C)c(C)n3)sc2c1)C(F)(F)F]

### Synthetic Scheme:

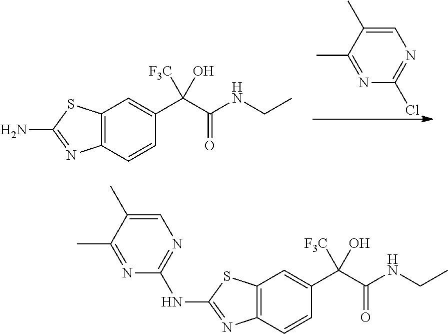

To 2-(2-aminobenzo[d]thiazol-6-yl)-N-ethyl-3,3,3-trifluoro-2-hydroxypropanamide (25 mg, 78 μmol, from Example 212) in dioxane (1.3 mL) was added 2-chloro-4,5-dimethylpyrimidine (9.8 μL, 71 μmol), potassium phosphate tribasic (25.4 mg, 0.12 mmol), tris(dibenzylideneacetone)-dipalladium (0) (7.17 mg, 7.8 μmol) and 4,4-bis(diphenylphosphino)-9,9-dimethylxanthene (13.6 mg, 24 μmol). The reaction was purged with nitrogen for 5 min and heated at 100° C. for 2 h. Then the reaction was filtered through Celite and concentrated. The crude product was dissolved in DMF and purified by reverse phase HPLC (C18, 0-100% MeCN/Ammonium bicarbonate 10 mM buffer) and lyophilized to afford the title compound (217, racemic) (2.0 mg, 6.0%) as an off-white solid. MS (ESI): mass calcd, for C₁₈H₁₈F₃N₅O₂S: 425.11, found: 426.2 [M+H]⁺. ¹H NMR (400 MHz, DMSO-d6) δ ppm 11.83 (s, 1H), 8.36 (s, 1H), 8.17-8.22 (m, 1H), 8.14 (s, 1H), 7.71 (s, 1H). 7.63 (s, 2H), 3.09-3.14 (m, 2H), 2.45 (s, 3H), 2.18 (s, 3H), 0.98 (t, J=7.1 Hz, 3H).

### Example 218. (R)-1-((S)-3,3,3-Trifluoro-2-(2-(3-(4-fluorophenyl)ureido)benzo[d]thiazol-6-yl)-2-hydroxypropanoyl)pyrrolidine-3-carboxylic acid (218)

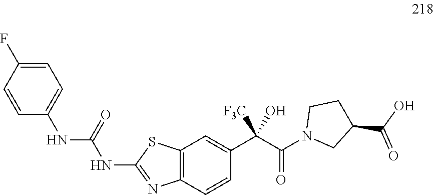 [SMILES: O=C(Nc1ccc(F)cc1)Nc1nc2ccc([C@](O)(C(=O)N3CC[C@@H](C(=O)O)C3)C(F)(F)F)cc2s1]

### Synthetic Scheme:

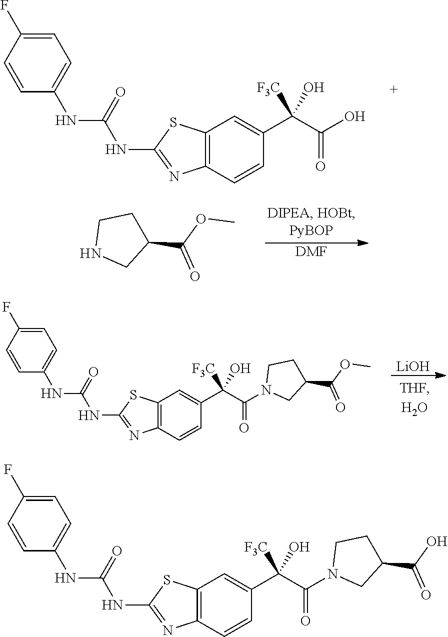

### (R)-Methyl 1-((S)-3,3,3-trifluoro-2-(2-(3-(4-fluorophenyl)ureido)benzo[d]thiazol-6-yl)-2-hydroxypropanoyl)pyrrolidine-3-carboxylate

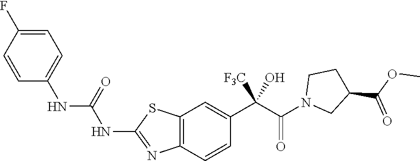 [SMILES: COC(=O)[C@@H]1CCN(C(=O)[C@@](O)(c2ccc3nc(NC(=O)Nc4ccc(F)cc4)sc3c2)C(F)(F)F)C1]

To a solution of (S)-3,3,3-trifluoro-2-(2-(3-(4-fluorophenyl)ureido)benzo[d]thiazol-6-yl)-2-hydroxypropanoic acid (50 mg, 0.12 mmol, see Example 38 for synthesis) and (R)-methyl pyrrolidine-3-carboxylate hydrochloride (57.9 mg, 0.35 mmol) in DMF (1.2 mL) was added PyBOP (186 mg, 0.35 mmol), HOBt (47.2 mg, 0.35 mmol), and DIPEA (0.16 mL, 0.93 mmol). After stirring at rt for 16 h, the reaction was directly purified by reverse phase HPLC (C18, 46% MeCN/Ammonium bicarbonate 10 mM buffer) to afford the title compound (30.0 mg, 46%). MS (ESI): mass calcd, for C₂₃H₂₀F₄N₄O₅S: 540.11, found: 541.1 [M+H]⁺.

### (R)-1-((S)-3,3,3-Trifluoro-2-(2-(3-(4-fluorophenyl)ureido)benzo[d]thiazol-6-yl)-2-hydroxypropanoyl)pyrrolidine-3-carboxylic Acid (218)

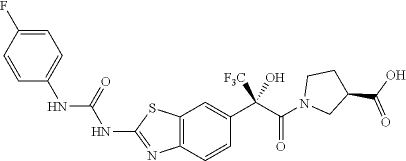 [SMILES: O=C(Nc1ccc(F)cc1)Nc1nc2ccc([C@](O)(C(=O)N3CC[C@@H](C(=O)O)C3)C(F)(F)F)cc2s1]

To stirred solution of (R)-methyl 1-((S)-3,3,3-trifluoro-2-(2-(3-(4-fluorophenyl)ureido)benzo[d]thiazol-6-yl)-2-hydroxypropanoyl)pyrrolidine-3-carboxylate (30 mg, 53 μmol) in THF (1.0 mL) was added lithium hydroxide (3.9 mg, 0.16 mmol) as a solution in water (100 μL). After stirring for 4 h, the reaction was quenched with 2 N aqueous HCl until the pH of the mixture was acidic. The reaction was extracted with DCM (2 mL×3). The combined organic layer was dried over Na₂SO₄, filtered, and concentrated to provide the title compound (218) (21 mg, 73%) as a white solid. MS (ESI): mass calcd. for C₂₂H₁₈F₄N₄O₅S: 526.09, found: 527.2 [M+H]⁺. ¹H NMR (400 MHz, DMSO-d6) δ ppm 9.38 (s, 1H), 8.02 (d, J=15.4 Hz, 1H), 7.93 (d, J=19.2 Hz, 1H), 7.61-7.71 (m, 1H), 7.51-7.59 (m, 2H), 7.43 (d, J=8.1 Hz, 1H), 7.13-7.21 (m, 2H), 3.33-3.83 (m, 3H), 2.52-3.17 (m, 2H), 1.64-1.99 (m, 2H).

Example 219 was synthesized following Example 218, using (S)-methyl pyrrolidine-3-carboxylate hydrochloride instead of (R)-methyl pyrrolidine-3-carboxylate hydrochloride.

### Example 220. (R)—N-Methyl-1-((S)-3,3,3-trifluoro-2-(2-(3-(4-fluorophenyl)ureido)benzo[d]thiazol-6-yl)-2-hydroxypropanoyl)pyrrolidine-3-carboxamide (220)

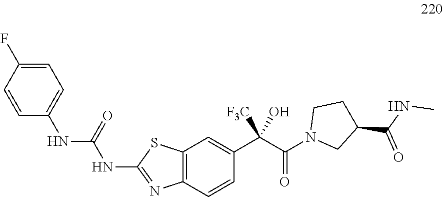 [SMILES: CNC(=O)[C@@H]1CCN(C(=O)[C@@](O)(c2ccc3nc(NC(=O)Nc4ccc(F)cc4)sc3c2)C(F)(F)F)C1]

### Synthetic Scheme:

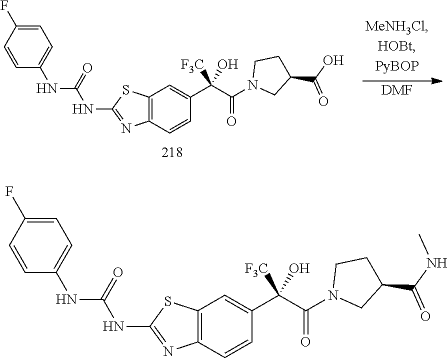

To a solution of (R)-1-((S)-3,3,3-trifluoro-2-(2-(3-(4-fluorophenyl)ureido)benzo[d]thiazol-6-yl)-2-hydroxypropanoyl)pyrrolidine-3-carboxylic acid (13.8 mg, 25 μmol, Example 218) and methylamine hydrochloride (5.3 mg, 76 μmol) in DMF (0.25 mL) were added DIPEA (35 μL, 0.2 mmol), HOBt (10.3 mg, 76.3 μmol), and PyBOP (40.5 mg, 76.3 μmol). After stirring for 4 h at rt, the reaction was directly purified by reverse phase HPLC (C18, 0-100% MeCN/Ammonium bicarbonate 10 mM buffer) to give the title compound (220) (4.9 mg, 35%) as a white solid. MS (ESI): mass calcd, for C₂₃H₂₁F₄N₅O₄S: 539.13, found: 540.3 [M+H]⁺. ¹H NMR (400 MHz, DMSO-d6) δ ppm 8.44 (s, 1H), 7.82-8.00 (m, 2H), 7.51-7.67 (m, 4H), 7.36 (t, J=9.1 Hz, 1H), 7.12 (t, J=8.2 Hz, 2H), 3.51-3.82 (m, 3H), 2.95-3.03 (m, 1H), 2.59-2.79 (m, 1H), 2.54 (d, J=4.5 Hz, 1.5H), 2.27 (d, J=4.5 Hz, 1.5H), 1.58-1.88 (m, 2H) (in rotamers).

Example 221 was synthesized in similar procedures as described in Example 220.

### Example 222. N-Ethyl-3,3,3-trifluoro-2-hydroxy-2-(2-(3-(pyrimidin-2-yl)ureido)benzo[d]thiazol-6-yl)propanamide (222)

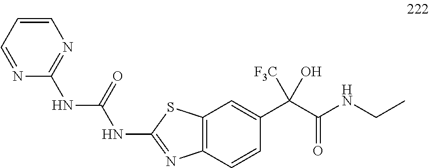 [SMILES: CCNC(=O)C(O)(c1ccc2nc(NC(=O)Nc3ncccn3)sc2c1)C(F)(F)F]

### Synthetic Scheme:

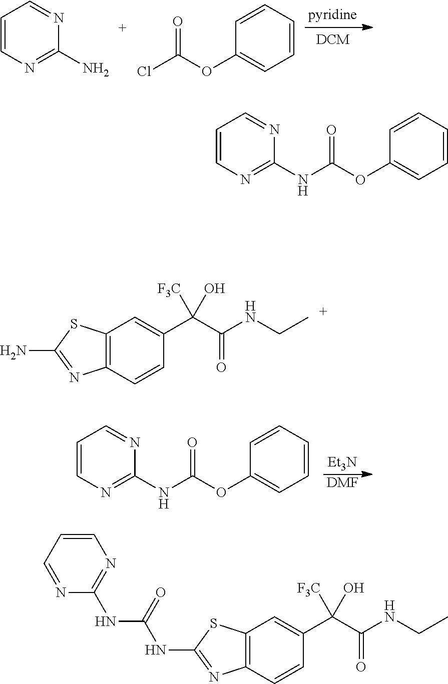

### Phenyl pyrimidin-2-ylcarbamate

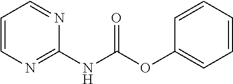 [SMILES: O=C(Nc1ncccn1)Oc1ccccc1]

To a solution of 2-aminopyrimidine (500 mg, 5.2 mmol) and pyridine (0.63 mL, 7.7 mmol) in DCM (5.2 mL) was added phenyl chloroformate (1.06 g, 6.7 mmol) and the solution was stirred for 16 h at rt. The reaction was quenched with saturated aqueous sodium bicarbonate solution (5 mL), diluted with DCM (10 mL), and extracted with DCM (20 mL×3). The combined organic layer was washed with water (20 mL), dried over Na₂SO₄, filtered, and concentrated to give the title compound (1.09 g, 85%). MS (ESI): mass calcd. for C₁₁H₉N₃O₂: 215.07, found: 216.1 [M+H]⁺.

### N-Ethyl-3,3,3-trifluoro-2-hydroxy-2-(2-(3-(pyrimidin-2-yl)ureido)benzo[d]thiazol-6-yl)propanamide (222)

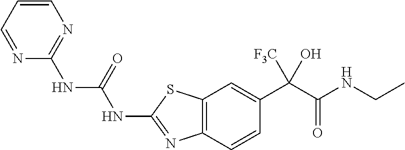 [SMILES: CCNC(=O)C(O)(c1ccc2nc(NC(=O)Nc3ncccn3)sc2c1)C(F)(F)F]

To a solution of 2-(2-aminobenzo[d]thiazol-6-yl)-N-ethyl-3,3,3-trifluoro-2-hydroxypropanamide (10 mg, 28 μmol) in DMF (0.28 mL) was added triethylamine (39 μL, 0.28 mmol) and phenyl pyrimidin-2-ylcarbamate (56.4 mg, 0.23 mmol) and the reaction was stirred at 65° C. for 4 h. Upon completion, the reaction was purified by reverse phase HPLC (C18, 45-65% MeCN/Ammonium formate 10 mM buffer) to provide the title compound (222) (2.7 mg, 21%, racemic) as an off-white solid. MS (ESI): mass calcd, for C₁₇H₁₅F₃N₆O₃S: 440.09, found: 441.3 [M+H]⁺. ¹H NMR (400 MHz, DMSO-d6) δ ppm 8.76 (d, J=4.9 Hz, 2H), 8.35 (s, 1H), 8.20-8.28 (m, 2H), 7.66-7.76 (m, 2H), 7.23 (t, J=4.9 Hz, 1H), 3.06-3.13 (m, 2H), 0.98 (t, J=7.2 Hz, 3H), (2 protons missing).

Example 223 was synthesized as a racemic mixture following Example 222, using 5-fluoropyrimidin-2-amine instead of 2-aminopyrimidine.

### Example 224. N-Ethyl-3,3,3-trifluoro-2-(2-(2-((4-fluorophenyl)amino)-2-oxoethyl)benzo[d]thiazol-6-yl)-2-hydroxypropanamide (224)

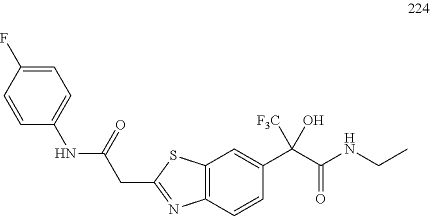 [SMILES: CCNC(=O)C(O)(c1ccc2nc(CC(=O)Nc3ccc(F)cc3)sc2c1)C(F)(F)F]

### Synthetic Scheme:

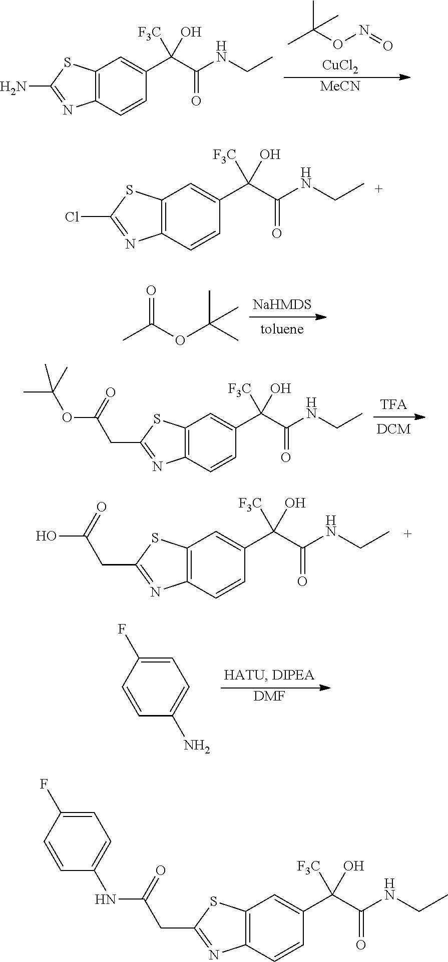

### 2-(2-Chlorobenzo[d]thiazol-6-yl)-N-ethyl-3,3,3-trifluoro-2-hydroxypropanamide

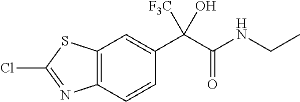 [SMILES: CCNC(=O)C(O)(c1ccc2nc(Cl)sc2c1)C(F)(F)F]

To a mixture of 2-(2-aminobenzo[d]thiazol-6-yl)-N-ethyl-3,3,3-trifluoro-2-hydroxypropanamide (220 mg, 0.69 mmol) and copper (II) chloride (111 mg, 827 μmol) in MeCN (8 mL) was added tert-butyl nitrite (137 μL, 1.0 mmol) at rt. The reaction was heated at 60° C. for 1.5 h, then the solvent was removed by concentration. The crude was purified by silica gel column chromatography (5-100% EtOAc/heptanes) to give the title compound (190 mg, 81%) as a pale-yellow oil. MS (ESI): mass calcd, for C₁₂H₁₀ClF₃N₂O₂S: 338.01, found: 337.0 [M−H]⁺.

### tert-Butyl 2-(6-(3-(ethylamino)-1,1,1-trifluoro-2-hydroxy-3-oxopropan-2-yl)benzo[d]thiazol-2-yl)acetate

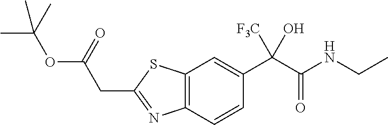 [SMILES: CCNC(=O)C(O)(c1ccc2nc(CC(=O)OC(C)(C)C)sc2c1)C(F)(F)F]

A solution of 2-(2-chlorobenzo[d]thiazol-6-yl)-N-ethyl-3,3,3-trifluoro-2-hydroxypropanamide (93 mg, 0.28 mmol) and tert-butyl acetate (0.18 mL, 1.37 mmol) in toluene (1.9 mL) was degassed with nitrogen for 5 min. To this solution at 0° C. was slowly added sodium bis(trimethylsilyl)amide solution (1.9 mL, 1.9 mmol), and the mixture was slowly raised to rt and stirred for 5 h. The reaction was quenched with aqueous NH₄Cl solution and extracted twice with EtOAc. The combined organic layer was washed with water and brine, dried over Na₂SO₄, filtered, and concentrated. The crude product was purified by silica gel column chromatography (5-100% EtOAc/heptanes) to give the title compound (95.0 mg, 83%) as a pale-yellow oil. MS (ESI): mass calcd, for C₁₈H₂₁F₃N₂O₄S: 418.12, found: 417.2 [M−H]⁻.

### 2-(6-(3-(Ethylamino)-1,1,1-trifluoro-2-hydroxy-3-oxopropan-2-yl)benzo[d]thiazol-2-yl)acetic Acid

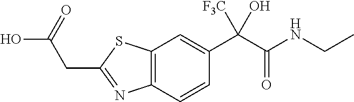 [SMILES: CCNC(=O)C(O)(c1ccc2nc(CC(=O)O)sc2c1)C(F)(F)F]

To a solution of tert-butyl 2-(6-(3-(ethylamino)-1,1,1-trifluoro-2-hydroxy-3-oxopropan-2-yl)benzo[d]thiazol-2-yl)acetate (10.0 mg, 23.9 μmol) in DCM (300 μL) was added trifluoroacetic acid (150 μL, 2.0 mmol) and the mixture was stirred at rt for 3 h. The solvent was evaporated under reduced pressure and the crude title residue was used directly in the next step without further purification. MS (ESI): mass calcd, for C₁₄H₁₃F₃N₂O₄S: 362.05, found: 363.1 [M+H]⁺.

### N-Ethyl-3,3,3-trifluoro-2-(2-(2-((4-fluorophenyl)amino)-2-oxoethyl)benzo[d]thiazol-6-yl)-2-hydroxypropanamide (224)

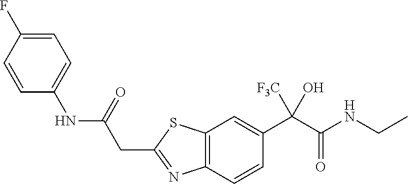 [SMILES: CCNC(=O)C(O)(c1ccc2nc(CC(=O)Nc3ccc(F)cc3)sc2c1)C(F)(F)F]

To a stirred solution of 2-(6-(3-(ethylamino)-1,1,1-trifluoro-2-hydroxy-3-oxopropan-2-yl)benzo[d]thiazol-2-yl)acetic acid (10.0 mg, 28 μmol) in DMF (285 μL) at 0° C. was added DIPEA (38.7 μL, 0.22 mmol) followed by 4-fluoroaniline (10.6 μL, 0.11 mmol) and then HATU (32.5 mg, 83 μmol). The reaction was stirred at rt for 17 h and purified by reverse phase column chromatography (C18, 5-100% MeCN/Ammonium formate 10 mM buffer) to give the title compound (224) (6.5 mg, 52%, racemic) as an off-white solid. MS (ESI): mass calcd, for C₂₀H₁₇F₄N₃O₃S: 455.09, found: 456.1 [M+H]⁺. ¹H NMR (400 MHz, DMSO-d6) δ ppm 10.54 (s, 1H), 8.36 (s, 1H), 8.28-8.33 (m, 1H), 7.97-8.03 (m, 1H), 7.76-7.84 (m, 1H), 7.63 (dd, J=9.0, 5.0 Hz, 2H), 7.17 (t, J=8.9 Hz, 2H), 4.30 (s, 2H), 3.06-3.17 (m, 2H), 0.98 (t, J=7.1 Hz, 3H).

### Example 225, 2-(2-(3-(Bicyclo[2.2.2]octan-1-yl)ureido)benzo[d]thiazol-6-yl)-N-ethyl-3,3,3-trifluoro-2-hydroxypropanamide (225)

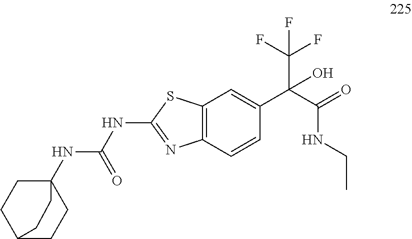 [SMILES: CCNC(=O)C(O)(c1ccc2nc(NC(=O)NC34CCC(CC3)CC4)sc2c1)C(F)(F)F]

### Synthetic Scheme:

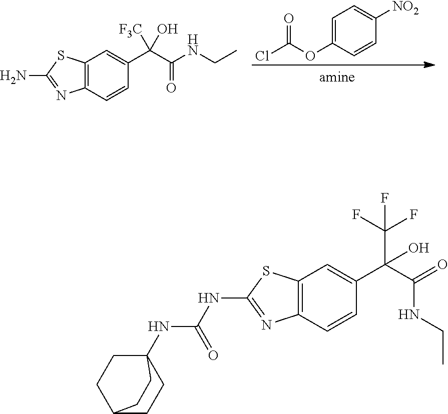

### 2-(2-(3-(Bicyclo[2.2.2]octan-1-yl)ureido)benzo[d]thiazol-6-yl)-N-ethyl-3,3,3-trifluoro-2-hydroxypropanamide (225)

To a solution of 2-(2-aminobenzo[d]thiazol-6-yl)-N-ethyl-3,3,3-trifluoro-2-hydroxypropanamide (50 mg, 0.16 mmol) in THF (1 mL) was added pyridine (127 μL, 1.6 mmol). After 5 min, 4-nitrophenyl chloroformate (63.1 mg, 313 μmol) was added. The reaction was stirred for 1.5 h at rt. Then a solution of bicyclo[2.2.2]octan-1-amine hydrochloride (78.3 mg, 0.47 mmol) in THF (0.2 mL) was neutralized with N,N-diisopropylethylamine (136 μL, 0.78 mmol) and added to the first solution. After heating at 60° C. for 1.5 h, the reaction was purified by reverse phase HPLC (C18, 5-100% MeCN/Ammonium formate 10 mM buffer) to afford the title compound (225) (22 mg, 30%) as a white solid. MS (ESI): mass calcd. for C₂₁H₂₅F₃N₄O₃S: 470.16, found: 471.1 [M+H]⁺. ¹H NMR (400 MHz, DMSO-d6) δ ppm 8.20 (t, J=5.9 Hz, 1H), 8.11 (s, 1H), 7.74 (s, 1H), 7.60-7.63 (m, 2H), 6.48 (s, 1H), 3.07-3.16 (m, 2H), 1.74-1.85 (m, 6H), 1.59-1.66 (m, 6H), 1.52-1.57 (m, 1H), 0.99 (t, J=7.1 Hz, 3H).

Example 226. Example 228, Example 229, Example 230, Example, and Example 232 were synthesized in similar procedures as described in Example 225. All are racemic.

### Example 227. 2-(2-((5-Cyclopropyl-1,3,4-thiadiazol-2-yl)amino)benzo[d]thiazol-6-yl)-N-ethyl-3,3,3-trifluoro-2-hydroxypropanamide (227)

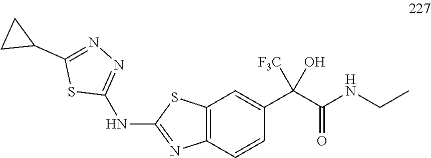 [SMILES: CCNC(=O)C(O)(c1ccc2nc(Nc3nnc(C4CC4)s3)sc2c1)C(F)(F)F]

### Synthetic Scheme:

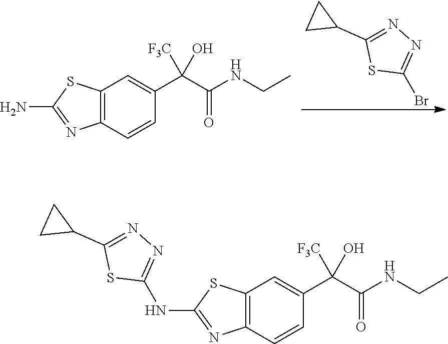 [SMILES: CCNC(=O)C(O)(c1ccc2nc(N)sc2c1)C(F)(F)F ; Brc1nnc(C2CC2)s1 ; CCNC(=O)C(O)(c1ccc2nc(Nc3nnc(C4CC4)s3)sc2c1)C(F)(F)F]

### 2-(2-((5-Cyclopropyl-1,3,4-thiadiazol-2-yl)amino)benzo[d]thiazol-6-yl)-N-ethyl-3,3,3-trifluoro-2-hydroxypropanamide (227)

To a solution of 2-(2-aminobenzo[d]thiazol-6-yl)-N-ethyl-3,3,3-trifluoro-2-hydroxypropanamide (50 mg, 0.16 mmol) in dioxane (2 mL) was added 2-bromo-5-cyclopropyl-1,3,4-thiadiazole (48.2 mg, 0.24 mmol) followed by 4,4-bis(diphenylphosphino)-9,9-dimethylxanthene (18.5 mg, 31 μmol), cesium carbonate (78.1 mg, 0.24 mmol) and palladium (II) acetate (5.0 mg, 22 μmol). The suspension was degassed with nitrogen and the reaction was heated at 100° C. for 17 h. After cooling down to rt, solvents were removed and the crude was purified by reverse phase HPLC (C18, 5-100% MeCN/Ammonium formate 10 mM buffer) to afford the title compound (227) (1.5 mg, 2%, racemic) as an off-white solid. MS (ESI): mass calcd, for C₁₇H₁₆F₃N₅O₂S₂: 443.07, found: 444.1 [M+H]⁺.

### Example 233. (R)—N-Ethyl-3,3,3-trifluoro-2-((R)-2-(3-(4-fluorophenyl)ureido)-4,5,6,7-tetrahydrobenzo[d]thiazol-6-yl)-2-hydroxypropanamide (233)

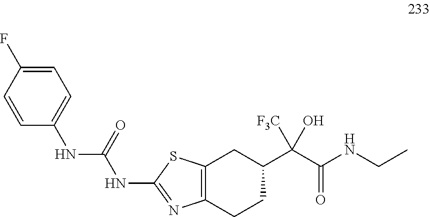 [SMILES: CCNC(=O)C(O)([C@@H]1CCc2nc(NC(=O)Nc3ccc(F)cc3)sc2C1)C(F)(F)F]

### Synthetic Scheme:

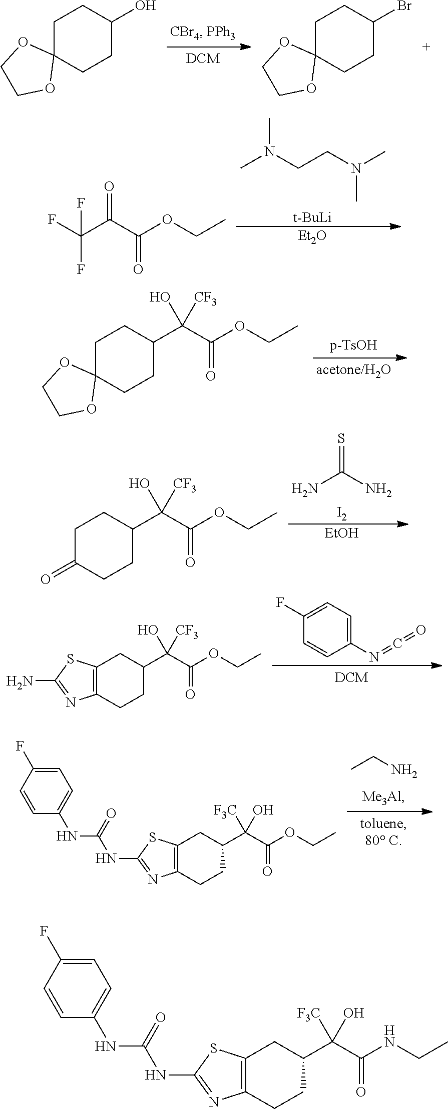

### 8-Bromo-1,4-dioxaspiro[4.5]decane

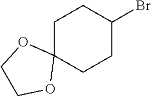 [SMILES: BrC1CCC2(CC1)OCCO2]

To a solution of 1,4-dioxaspiro[4.5]decan-8-ol (5.0 g, 31.0 mmol) in DCM (31 mL) at 0° C. was added carbon tetrabromide (11.3 g, 34.1 mmol) followed by triphenylphosphine (9.03 g, 34.1 mmol) and the mixture was stirred for 17 h at rt. The reaction was concentrated onto silica gel and purified by silica gel column chromatography (0-10% EtOAc/heptanes) to give the title compound (2.8 g, 41%). ¹H NMR (400 MHz, CDCl₃) δ ppm 4.32 (tt, J=7.1, 3.7 Hz, 1H), 3.87-4.00 (m, 4H), 2.02-2.21 (m, 4H), 1.85-1.97 (m, 2H), 1.61 (ddd, J=12.8, 7.9, 4.6 Hz, 2H).

### Ethyl 3,3,3-trifluoro-2-hydroxy-2-(1,4-dioxaspiro[4.5]decan-8-yl)propanoate

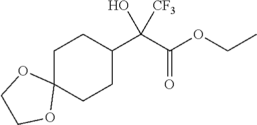 [SMILES: CCOC(=O)C(O)(C1CCC2(CC1)OCCO2)C(F)(F)F]

In a flame dried flask under nitrogen, tert-butyllithium 1.7 M in pentane (5.3 mL, 9.1 mmol) was added dropwise to a solution of 8-bromo-1,4-dioxaspiro[4.5]decane (1.0 g, 4.5 mmol) and N,N,N,N′-tetramethylethylenediamine (1.7 mL, 9.1 mmol) in diethyl ether (22.6 mL) at −78° C. Then ethyl trifluoropyruvate (1.3 mL, 9.1 mmol) was added dropwise. The reaction was stirred for 30 min at −78° C., quenched with water, warmed to rt and extracted with EtOAc (3×). The combined organic layer was washed with brine, dried over Na₂SO₄, filtered, and concentrated. The crude was purified by silica gel column chromatography (0-50% EtOAc/heptanes) to provide the title compound (480 mg, 34%). ¹H NMR (400 MHz, CDCl₃) δ ppm 4.30-4.44 (m, 2H), 3.93 (s, 4H), 2.10 (tt, J=12.2, 3.4 Hz, 1H), 1.48-1.89 (m, 8H), 1.35 (t, J=7.1 Hz, 3H).

### Ethyl 3,3,3-trifluoro-2-hydroxy-2-(4-oxocyclohexyl)propanoate

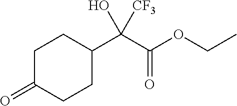 [SMILES: CCOC(=O)C(O)(C1CCC(=O)CC1)C(F)(F)F]

To a solution of ethyl 3,3,3-trifluoro-2-hydroxy-2-(1,4-dioxaspiro[4.5]decan-8-yl)propanoate (480 mg, 1.5 mmol) in acetone (10.2 mL) and water (5.1 mL) was added p-toluenesulfonic acid monohydrate (29.7 mg, 0.15 mmol). After stirring for 17 h at rt, the reaction was extracted with DCM (3×). The combined organic layer was washed with saturated aqueous NaHCO₃ solution, and brine, dried over Na₂SO₄, filtered, and concentrated. The crude was purified by silica gel column chromatography (20-60% EtOAc/heptanes) to give the title compound (165 mg, 40%). ¹H NMR (400 MHz, CDCl₃) δ ppm 4.31-4.48 (m, 2H), 3.86 (s, 1H), 2.29-2.61 (m, 5H), 2.14-2.2 (m, 1H), 1.73-1.89 (m, 2H), 1.64 (ddq, J=12.6, 6.4, 3.3 Hz, 1H), 1.36 (t, J=7.1 Hz, 3H).

### Ethyl 2-(2-amino-4,5,6,7-tetrahydrobenzo[d]thiazol-6-yl)-3,3,3-trifluoro-2-hydroxypropanoate

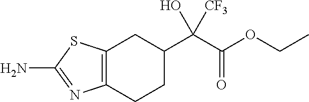 [SMILES: CCOC(=O)C(O)(C1CCc2nc(N)sc2C1)C(F)(F)F]

A solution of ethyl 3,3,3-trifluoro-2-hydroxy-2-(4-oxocyclohexyl)propanoate (100 mg, 0.37 mmol), thiourea (85.1 mg, 1.12 mmol) and iodine (94.6 mg, 0.37 mmol) in EtOH (1.2 mL) was refluxed for 17 h and then cooled down to rt. Water was added and the mixture was extracted with DCM (3×), dried over Na₂SO₄, filtered, and concentrated. The crude was purified by silica gel column chromatography (50-100% EtOAc/heptanes) to provide the title compound (50 mg, 41%). MS (ESI): mass calcd, for C₁₂H₁₅F₃N₂O₃S: 324.08, found: 325.3 [M+H]⁺.

### Ethyl 3,3,3-trifluoro-2-((R)-2-(3-(4-fluorophenyl)ureido)-4,5,6,7-tetrahydrobenzo[d]thiazol-6-yl)-2-hydroxypropanoate and ethyl 3,3,3-trifluoro-2-((S)-2-(3-(4-fluorophenyl)ureido)-4,5,6,7-tetrahydrobenzo[d]thiazol-6-yl)-2-hydroxypropanoate

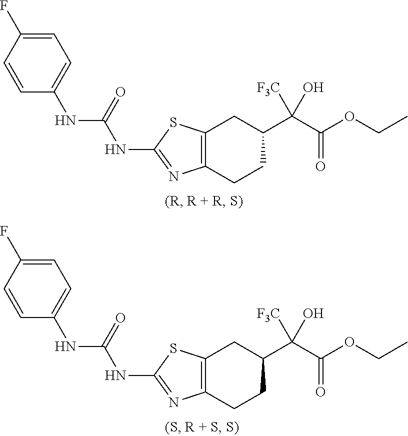 [SMILES: CCOC(=O)C(O)([C@@H]1CCc2nc(NC(=O)Nc3ccc(F)cc3)sc2C1)C(F)(F)F ; CCOC(=O)C(O)([C@H]1CCc2nc(NC(=O)Nc3ccc(F)cc3)sc2C1)C(F)(F)F]

To a solution of ethyl 2-(2-amino-4,5,6,7-tetrahydrobenzo[d]thiazol-6-yl)-3,3,3-trifluoro-2-hydroxypropanoate (50 mg, 0.15 mmol) in DCM (7.7 mL) was added 4-fluorophenyl isocyanate (32 mg, 0.23 mmol). After stirring for 1 h, solvent was removed and the crude was purified by silica gel column chromatography (25-70% EtOAc/heptanes) to provide 2 pairs of diastereomers: the title compounds as a mixture of R,R and R,S (27 mg, 38%) as well as a mixture of S, R and S, S diastereomer (20.0 mg, 28%)—both racemic at this point. Stereochemistry was arbitrarily assigned. MS (ESI): mass calcd, for C₁₉H₁₉F₄N₃O₄S: 461.10 found: 462.4 [M+H]⁺.

### (R)—N-Ethyl-3,3,3-trifluoro-2-((R)-2-(3-(4-fluorophenyl)ureido)-4,5,6,7-tetrahydrobenzo[d]thiazol-6-yl)-2-hydroxypropanamide (233)

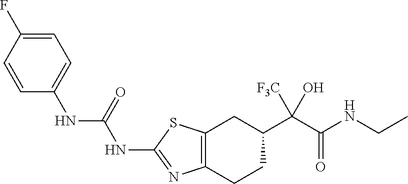 [SMILES: CCNC(=O)C(O)([C@@H]1CCc2nc(NC(=O)Nc3ccc(F)cc3)sc2C1)C(F)(F)F]

To a solution of ethyl amine hydrochloride (48.7 mg, 0.59 mmol) in toluene (1.2 mL) at 0° C. was slowly added trimethylaluminum (2 M in toluene) (0.29 mL, 0.59 mmol) and the reaction was stirred at 0° C. for 30 min and then at rt for 1 h. Racemic ethyl 3,3,3-trifluoro-2-((R)-2-(3-(4-fluorophenyl)ureido)-4,5,6,7-tetrahydrobenzo[d]thiazol-6-yl)-2-hydroxypropanoate (27 mg, 0.59 mmol) in solution in toluene (1.2 mL) was added and the mixture was heated at 80° C. for 17 h. The reaction was cooled down to rt and sodium sulfate decahydrate was added until no more bubbling was observed. The solid was filtered and the filtrate was concentrated. The crude was purified by reverse phase HPLC (C18, 10-60% MeCN/Ammonium formate 10 mM buffer) to give the title compound (233) (10.0 mg, 37%) as a white solid. Stereochemistry was arbitrarily assigned. MS (ESI): mass calcd. for C₁₉H₂₀F₄N₄O₃S: 460.12, found: 461.4 [M+H]⁺. ¹H NMR (400 MHz, DMSO-d6) δ ppm 9.39-9.60 (m, 1H), 8.22 (t, J=5.9 Hz, 1H), 7.48 (dd, J=9.0, 4.9 Hz, 2H), 7.11 (t, J=8.8 Hz, 2H), 6.80 (s, 1H), 3.51 (t, J=7.7 Hz, 1H), 3.10-3.19 (m, 2H), 2.58-2.75 (m, 2H), 2.36-2.44 (m, 1H), 1.93-2.05 (m, 1H), 1.53-1.75 (m, 2H), 1.02 (t, J=7.1 Hz, 3H).

Example 234 was synthesized in similar procedures as Example 233, using the second set of diastereoisomer from above. Stereochemistry was arbitrarily assigned.

### Example 235. N-(2-(Dimethylamino)ethyl)-3,3,3-trifluoro-2-(2-(3-(4-fluorophenyl)ureido)benzo[d]thiazol-6-yl)-2-hydroxypropanamide (235)

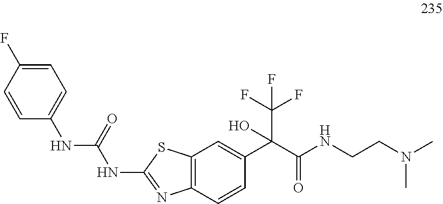 [SMILES: CN(C)CCNC(=O)C(O)(c1ccc2nc(NC(=O)Nc3ccc(F)cc3)sc2c1)C(F)(F)F]

### Synthetic Scheme:

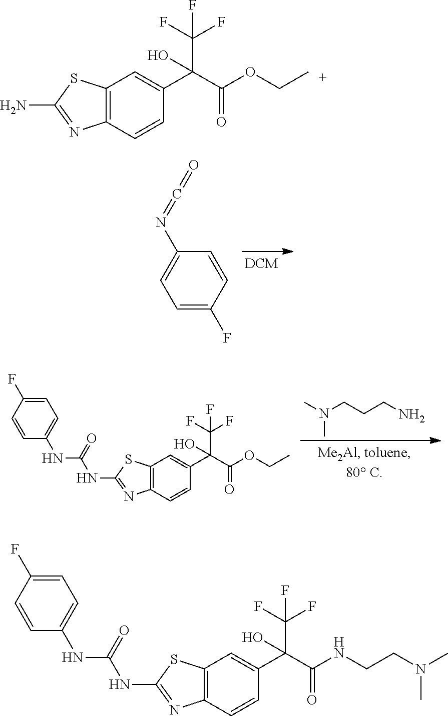

### Ethyl 3,3,3-trifluoro-2-(2-(3-(4-fluorophenyl)ureido)benzo[d]thiazol-6-yl)-2-hydroxypropanoate

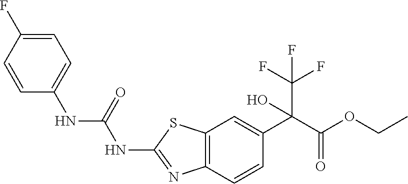 [SMILES: CCOC(=O)C(O)(c1ccc2nc(NC(=O)Nc3ccc(F)cc3)sc2c1)C(F)(F)F]

To a solution of ethyl 2-(2-aminobenzo[d]thiazol-6-yl)-3,3,3-trifluoro-2-hydroxypropanoate (40.0 mg, 0.13 mmol) in DCM (6.2 mL) was added 4-fluorophenyl isocyanate (25.9 mg, 0.19 mmol) and stirred for 1 h at rt. Solvent was removed by concentration and the product was purified by silica gel column chromatography (20-80% EtOAc/heptanes) to give the title compound title (25 mg, 44%) as an off-white solid. MS (ESI): mass calcd, for C₁₉H₁₅F₄N₃O₄S: 457.07, found: 458.4 [M+H]⁺.

### N-(2-(Dimethylamino)ethyl)-3,3,3-trifluoro-2-(2-(3-(4-fluorophenyl)ureido)benzo[d]thiazol-6-yl)-2-hydroxypropanamide (235)

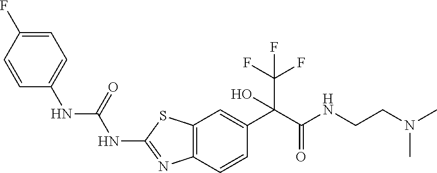 [SMILES: CN(C)CCNC(=O)C(O)(c1ccc2nc(NC(=O)Nc3ccc(F)cc3)sc2c1)C(F)(F)F]

To a solution of N,N-dimethylethylenediamine (62.8 μL, 0.55 mmol) in toluene (1.1 mL) at 0° C. was slowly added trimethylaluminum (2 M in toluene, 273 μL, 0.55 mmol) and the mixture was stirred at 0° C. for 30 min and then at rt for 1 h. Ethyl 3,3,3-trifluoro-2-(2-(3-(4-fluorophenyl)ureido)benzo[d]thiazol-6-yl)-2-hydroxypropanoate (25 mg, 0.055 mmol) in solution in toluene (1.1 mL) was added to the reaction and the mixture was heated at 80° C. for 17 h. The reaction was cooled down to rt and sodium sulfate decahydrate was added until no more bubbling was observed. The solid was filtered and the filtrate was concentrated. The crude was purified by reverse phase HPLC (C18, 60% MeCN/Ammonium formate 10 mM buffer) to give the title compound (235) (13 mg, 48%, racemic) as a light yellow solid. MS (ESI): mass calcd, for C₁₉H₂₀F₄N₄O₃S: 499.13, found: 500.4 [M+H]⁺. ¹H NMR (400 MHz, DMSO-d6) δ ppm 8.21 (s, 1H), 8.14 (s, 1H), 8.06 (t, J=5.6 Hz, 1H), 7.64 (s, 2H), 7.54 (dd, J=9.1, 4.9 Hz, 2H), 7.15 (t, J=8.9 Hz, 2H), 3.15-3.25 (m, 2H), 2.30 (t, J=6.3H), 2.12 (s, 6H).

### Example 236. (S)-3,3,3-Trifluoro-N-((1s,3R)-3-fluorocyclobutyl)-2-(2-(3-(4-fluorophenyl)ureido)benzo[d]thiazol-6-yl)-2-hydroxypropanamide (236)

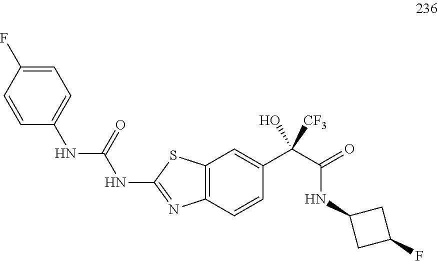 [SMILES: O=C(Nc1ccc(F)cc1)Nc1nc2ccc([C@](O)(C(=O)N[C@H]3C[C@@H](F)C3)C(F)(F)F)cc2s1]

To a solution of (S)-3,3,3-trifluoro-2-(2-(3-(4-fluorophenyl)ureido)benzo[d]thiazol-6-yl)-2-hydroxypropanoic acid (50 mg, 0.12 mmol) and cis-3-fluorocyclobutanamine hydrochloride (45.2 mg, 0.35 mmol) in DMF (1.2 mL) was added HOBt (47.2 mg, 0.35 mmol), DIPEA (164 μL, 0.93 mmol) and PyBOP (186 mg, 0.35 mmol) at rt and the solution was stirred for 16 h. The reaction mixture was directly purified by reverse phase HPLC (C18, 60% MeCN/Ammonium bicarbonate 10 mM) to afford the title compound (236) (35 mg, 60%) as a white solid. MS (ESI): mass calcd, for C₂₁H₁₇F₅N₄O₃S: 500.09, found: 501.2 [M+H]⁺. ¹H NMR (400 MHz, DMSO-d6) δ ppm 9.23 (s, 1H), 8.52 (d, J=7.8 Hz, 1H), 8.14 (s, 1H), 7.82 (s, 1H), 7.63 (s, 2H), 7.48-7.55 (m, 2H), 7.12-7.18 (m, 2H), 4.83 (p. J=6.9 Hz, 0.5H), 4.69 (p, J=6.9 Hz, 0.5H), 3.78 (h, J=7.7 Hz, 1H), 2.58-2.67 (m, 1H), 2.50-2.58 (m, 1H), 2.09-2.38 (m, 2H).

Example 237 was synthesized following Example 236, using trans-3-fluorocyclobutanamine hydrochloride instead of cis-3-fluorocyclobutanamine hydrochloride.

Example 238, and Example 239 were synthesized in similar procedures as described in Example 236. Ammonium formate was used in HPLC purification for both compounds.

### Example 240: 1-(4-Fluorophenyl)-3-(6-(1,1,1-trifluoro-3-hydroxy-2-(hydroxymethyl) propan-2-yl)benzo[d]thiazol-2-yl)urea (240)

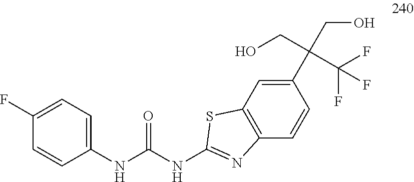 [SMILES: O=C(Nc1ccc(F)cc1)Nc1nc2ccc(C(CO)(CO)C(F)(F)F)cc2s1]

### Synthetic Scheme:

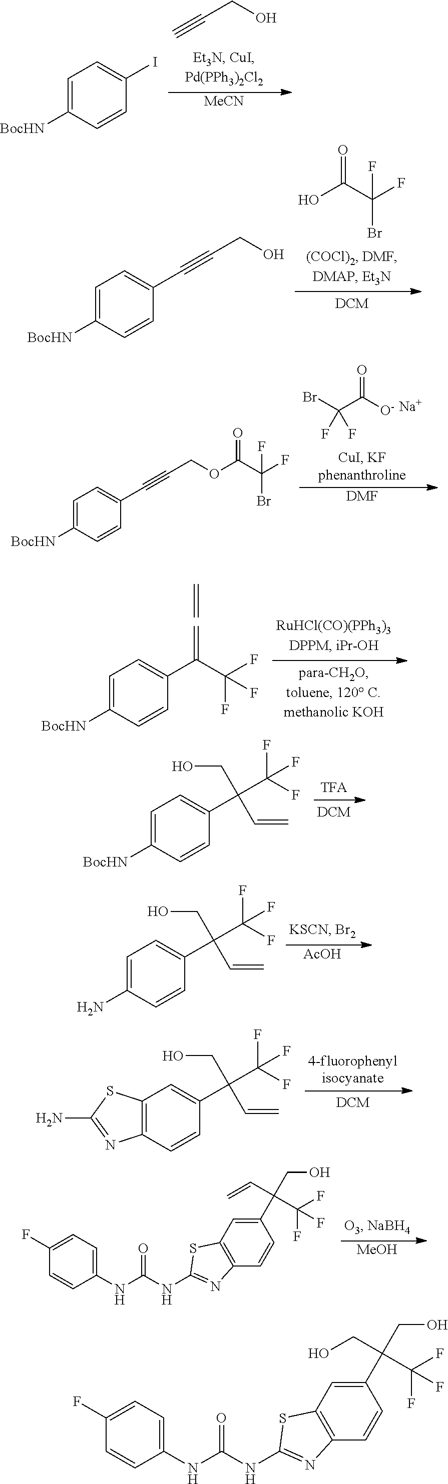

### tert-Butyl (4-(3-hydroxyprop-1-yn-1-yl)phenyl)carbamate

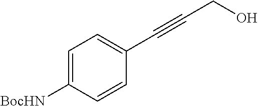 [SMILES: CC(C)(C)OC(=O)Nc1ccc(C#CCO)cc1]

To a solution of CuI (119 mg, 627 μmol), Pd(PPh₃)₂Cl₂ (224 mg, 313 μmol) and N-Boc-4-iodoaniline (1.0 g, 3.13 mmol) in ACN (3.1 mL) at 0° C. was added triethylamine (2.0 mL, 14.1 mmol). After stirring for 10 min, 2-propyn-1-ol (237 μL, 4.1 mmol) was added and the mixture was stirred at rt for 16 h. The reaction was quenched with saturated aqueous ammonium chloride solution (20 mL), diluted with EtOAc (20 mL), and extracted with EtOAc (20 mL×3). The combined organic layer was washed with water (20 mL) and brine (20 mL), dried over Na₂SO₄, filtered and concentrated. The crude residue was purified by silica gel column chromatography (24% EtOAc/heptanes) to afford the title compound (660 mg, 85%). ¹H NMR (400 MHz, CDCl₃) δ ppm 7.29-7.39 (m, 4H), 4.48 (s, 2H), 1.51 (s, 9H).

### 3-(4-((tert-Butoxycarbonyl)amino)phenyl) prop-2-yn-1-yl 2-bromo-2,2-difluoroacetate

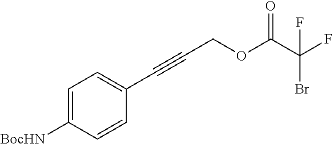 [SMILES: CC(C)(C)OC(=O)Nc1ccc(C#CCOC(=O)C(F)(F)Br)cc1]

Dimethylformamide (62.1 μL, 0.8 mmol) and oxalyl chloride (300 μL, 3.47 mmol) were added to a solution of bromodifluoroacetic acid (667 mg, 3.74 mmol) in DCM (13.3 mL) and the reaction was stirred at rt for 2 h. In a separate flask, tert-butyl (4-(3-hydroxyprop-1-yn-1-yl)phenyl)carbamate (660 mg, 2.67 mmol) was added to a solution of triethylamine (744 μL, 5.34 mmol) and 4-dimethylaminopyridine (66.5 mg, 0.53 mmol) in DCM (13.3 mL). This mixture was cooled to 0° C. and the solution of acid chloride was added dropwise. The reaction was warmed to rt and stirred for 16 h. Upon completion, the reaction was quenched by the addition of 1 N aqueous HCl (10 mL). The layers were separated and the aqueous layer was extracted with DCM (3×20 mL). The combined organic layer was washed with water (20 mL), dried over Na₂SO₄, filtered and concentrated. The crude residue was purified by silica gel column chromatography (12% EtOAc/heptanes) to give the title compound (690 mg, 64%). ¹H NMR (400 MHz, CDCl₃) δ ppm 7.37-7.43 (m, 2H), 7.34 (d, J=8.8 Hz, 2H), 5.14 (s, 2H), 1.52 (s, 9H).

### tert-Butyl (4-(1,1,1-trifluorobuta-2,3-dien-2-yl)phenyl)carbamate

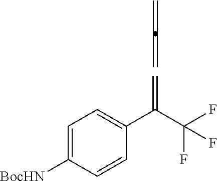

Potassium fluoride (129 mg, 2.2 mmol) was flame-dried in a microwave vial, followed by the addition of sodium bromodifluoroacetate (55.3 mg, 0.28 mmol), CuI (20.9 mg, 0.11 mmol), 1,10-phenanthroline (20.0 mg, 0.11 mmol) and anhydrous DMF (1.1 mL) (dried over flame-activated 4 Å MS). The reaction was heated to 50° C. and stirred for 10 min before the addition of 3-(4-((tert-butoxy carbonyl)amino)phenyl) prop-2-yn-1-yl 2-bromo-2,2-difluoroacetate (445 mg, 1.1 mmol), After heating at 50° C. for 16 h, the reaction was quenched with 1 N aqueous HCl solution (15 mL), diluted with EtOAc (25 mL) and extracted with EtOAc (5 mL×3). The combined organic layer was washed with water (25 mL) and brine (15 mL), dried over Na₂SO₄, filtered and concentrated. The crude residue was purified by silica gel column chromatography (8% EtOAc/heptanes) to give the title compound (140 mg, 43%). 1H NMR (400 MHz, CDCl₃) δ ppm 7.37 (d, J=9.4 Hz, 4H), 6.49 (s, 1H), 5.52 (q, J=3.4 Hz, 2H), 1.52 (s, 9H).

### tert-Butyl (4-(1,1,1-trifluoro-2-(hydroxymethyl)but-3-en-2-yl)phenyl)carbamate

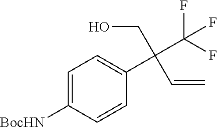 [SMILES: C=CC(CO)(c1ccc(NC(=O)OC(C)(C)C)cc1)C(F)(F)F]

To a solution of carbonylchlorohydridotris(triphenylphosphine)ruthenium (II) (22.5 mg, 23 μmol), bis(diphenylphosphino) methane (9.27 mg, 23 μmol) and paraformaldehyde (28.1 mg, 0.94 mmol) in toluene (1 mL) was added tert-butyl (4-(1,1,1-trifluorobuta-2,3-dien-2-yl)phenyl)carbamate (140 mg, 0.47 mmol) and 2-propanol (143 μL, 1.87 mmol). The reaction was heated to 120° C. and stirred for 1 h. After cooling to rt, KOH (61.8 mg, 0.94 mmol) dissolved in 0.23 mL of MeOH (4 M) was added and the reaction was stirred for another 16 h. Upon completion, the reaction was concentrated and diluted with EtOAc (10 mL), quenched with 1 N aqueous HCl (10 mL) and extracted with EtOAc (10 mL×3). The combined organic layer was washed with water (10 mL) and brine (10 mL), dried over Na₂SO₄, filtered and concentrated. The crude residue was purified by silica gel column chromatography (15% EtOAc/heptanes) to give the title compound (137 mg, 88%). ¹H NMR (400 MHz, CDCl₃) δ ppm 7.39 (d, J=3.0 Hz, 4H), 6.46-6.51 (m, 1H), 6.09 (s, 1H), 5.59 (d, J=11.5 Hz, 1H), 5.38 (d, J=17.8 Hz, 1H), 4.17-4.24 (m, 1H), 4.08-4.16 (m, 1H), 1.52 (s, 9H).

### 2-(4-Aminophenyl)-2-(trifluoromethyl) but-3-en-1-ol

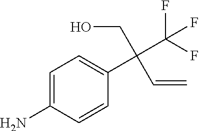 [SMILES: C=CC(CO)(c1ccc(N)cc1)C(F)(F)F]

To a solution of tert-butyl (4-(1,1,1-trifluoro-2-(hydroxymethyl) but-3-en-2-yl)phenyl)carbamate (800 mg, 2.4 mmol) in DCM (24 mL) was added trifluoroacetic acid (1.9 mL, 24 mmol) and the reaction was allowed to stir for 16 h at rt. Upon completion, the reaction mixture was concentrated and diluted with methanol (100 mL). The reaction was basified with sodium carbonate until pH>8. The reaction was stirred for another 30 min, filtered, and concentrated to give the title compound (550 mg, 99%). ¹H NMR (400 MHz, DMSO-d6) δ ppm 7.06 (d, J=8.7 Hz, 2H), 6.51 (d, J=8.7 Hz, 2H), 5.98 (dd, J=17.9, 11.2 Hz, 1H), 5.42 (d, J=11.4 Hz, 1H), 5.24 (d, J=17.8 Hz, 1H), 4.09 (s, 2H), 3.92 (q, J=10.9 Hz, 2H).

### 2-(2-Aminobenzo[d]thiazol-6-yl)-2-(trifluoromethyl) but-3-en-1-ol

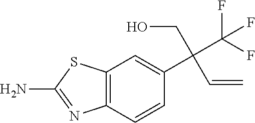 [SMILES: C=CC(CO)(c1ccc2nc(N)sc2c1)C(F)(F)F]

To a stirred solution of 2-(4-aminophenyl)-2-(trifluoromethyl) but-3-en-1-ol (550 mg, 2.4 mmol) in AcOH (11.9 mL) was added potassium thiocyanate (925 mg, 951 mmol) followed by the dropwise addition of bromine (122 μL, 2.4 mmol) at rt and the mixture was stirred for 16 h. Upon completion, the reaction mixture was diluted with EtOAc (50 mL), quenched with saturated aqueous sodium thiosulfate solution (50 mL), stirred for another 10 min and extracted with EtOAc (50 mL×3). The combined organic layer was washed with saturated aqueous sodium bicarbonate solution (50 mL), water (50 mL) and brine (50 mL), dried over Na₂SO₄, filtered, and concentrated. The resulting crude residue was purified by silica gel column chromatography (50% EtOAc/heptanes) to give the title compound (600 mg, 87%). MS (ESI): mass calcd, for C₁₂H₁₁F₃N₂OS: 288.05, found: 289.1 [M+H]⁺.

### 1-(4-Fluorophenyl)-3-(6-(1,1,1-trifluoro-2-(hydroxymethyl) but-3-en-2-yl)benzo[d]thiazol-2-yl)urea

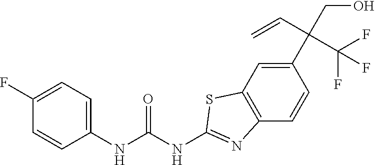 [SMILES: C=CC(CO)(c1ccc2nc(NC(=O)Nc3ccc(F)cc3)sc2c1)C(F)(F)F]

A solution of 2-(2-aminobenzo[d]thiazol-6-yl)-2-(trifluoromethyl) but-3-en-1-ol (600 mg, 2.1 mmol) and 4-fluorophenyl isocyanate (432 mg, 3.1 mmol) in DCM (20.8 mL) was stirred at rt for 4 h. The reaction was concentrated and purified by silica gel column chromatography (30% EtOAc/heptanes) to give the title compound (200 mg, 23%). MS (ESI): mass calcd, for C₁₉H₁₅F₄N₃O₂S: 425.08, found: 426.2 [M+H]⁺.

### 1-(4-Fluorophenyl)-3-(6-(1,1,1-trifluoro-3-hydroxy-2-(hydroxymethyl) propan-2-yl)benzo[d]thiazol-2-yl)urea (240)

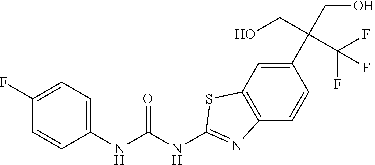 [SMILES: O=C(Nc1ccc(F)cc1)Nc1nc2ccc(C(CO)(CO)C(F)(F)F)cc2s1]

To a solution of 1-(4-fluorophenyl)-3-(6-(1,1,1-trifluoro-2-(hydroxymethyl) but-3-en-2-yl)benzo[d]thiazol-2-yl)urea (150 mg, 0.35 mmol) in MeOH (3.5 mL) at −78° C., ozone was bubbled through for 1 h. The reaction was warmed to rt and sodium borohydride (139 mg, 3.53 mmol) was added slowly. After stirring for 16 h, the reaction was concentrated, diluted with EtOAc (10 mL), quenched with water (10 mL) and extracted with EtOAC (10 mL×3). The combined organic layer was washed with water (10 mL) and brine (10 mL), dried with Na₂SO₄, filtered, and concentrated. The crude was purified by silica gel column chromatography to give the title compound (240) (150 mg, 95%) as a white solid. MS (ESD): mass calcd, for C₁₈H₁₅F₄NO₃S: 429.06, found: 430.1 [M+H]⁺. ¹H NMR (400 MHz, DMSO-d6) δ ppm 10.77 (br s, 1H), 9.18 (br s, 1H), 8.05 (s, 1H), 7.61 (s, 1H), 7.53 (d, J=8.1 Hz, 3H), 7.16 (t, J=8.9 Hz, 2H), 5.06 (t, J=5.3 Hz, 2H), 4.17 (dd, J=11.2, 5.2 Hz, 2H), 3.97 (dd, J=11.3, 5.4 Hz, 2H).

### Example 241. N-Ethyl-3,3,3-trifluoro-2-(2-(3-(4-fluorophenyl)ureido)benzo[d]thiazol-6-yl)-2-(hydroxymethyl)propanamide

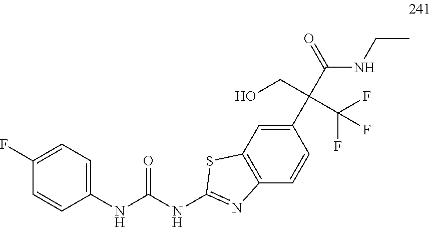 [SMILES: CCNC(=O)C(CO)(c1ccc2nc(NC(=O)Nc3ccc(F)cc3)sc2c1)C(F)(F)F]

### Synthetic Scheme:

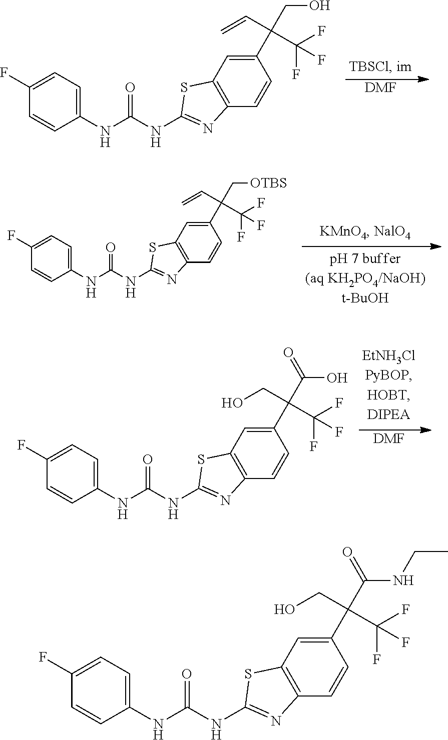

### 1-(6-(2-(((ter-Butyldimethylsilyl)oxy)methyl)-1,1,1-trifluorobut-3-en-2-yl)benzo[d]thiazol-2-yl)-3-(4-fluorophenyl)urea

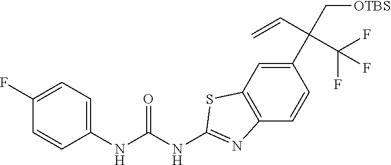 [SMILES: C=CC(CO[Si](C)(C)C(C)(C)C)(c1ccc2nc(NC(=O)Nc3ccc(F)cc3)sc2c1)C(F)(F)F]

To a solution of 1-(4-fluorophenyl)-3-(6-(1,1,1-trifluoro-2-(hydroxymethyl) but-3-en-2-yl)benzo[d]thiazol-2-yl)urea (200 mg, 0.47 mmol) in DMF (2 mL) was sequentially added imidazole (97 mg, 1.4 mmol) and tert-butyldimethylsilyl chloride (99 μL, 0.56 mmol). After stirring at ambient temperature for 16 h, the reaction was quenched with water (5 mL), diluted with EtOAc (5 mL) and extracted with EtOAc (5 mL×3). The combined organic layer was washed with water (5 mL) and brine (5 mL), dried over sodium sulfate, and concentrated. The crude residue was purified by silica gel column chromatography (30% EtOAc/heptanes) to afford the title compound (240 mg, 95%). MS (ESI): mass calcd, for C₂₅H₂₉F₄N₃O₂SSi: 539.17, found: 540.2 [M+H]⁺.

### 3,3,3-Trifluoro-2-(2-(3-(4-fluorophenyl)ureido)benzo[d]thiazol-6-yl)-2-(hydroxymethyl)propanoic Acid

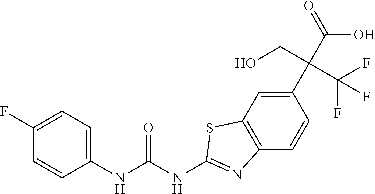 [SMILES: O=C(Nc1ccc(F)cc1)Nc1nc2ccc(C(CO)(C(=O)O)C(F)(F)F)cc2s1]

To a solution of sodium periodate (641 mg, 3.0 mmol) in ready-to-use (aqueous phosphate mixture) pH 7 buffer (18.5 mL) was added potassium permanganate (59.2 mg, 0.37 mmol) at ambient temperature and stirred for 15 min. A solution of 1-(6-(2-(((tert-butyldimethylsilyl)oxy)methyl)-1,1,1-trifluorobut-3-en-2-yl)benzo[d]thiazol-2-yl)-3-(4-fluorophenyl)urea (200 mg, 0.37 mmol) in t-BuOH (18.5 mL) was added and the reaction was stirred for another 16 h. The reaction was quenched with sodium thiosulfate pentahydrate (178 mg, 1.1 mmol), stirred for 1 h at rt, diluted with EtOAc (100 mL) and water (100 mL) and extracted with EtOAc (100 mL×3). The combined organic layer was washed with water (100 mL) and brine (100 mL), dried over sodium sulfate and concentrated. The crude was purified using reverse phase HPLC (C18, 40% MeCN/Ammonium bicarbonate 10 mM buffer) to afford the title compound (75 mg, 45%). MS (ESI): mass calcd, for C₁₈H₁₃F₄N₃O₄S: 443.06, found: 444.2 [M+H]⁺.

### N-Ethyl-3,3,3-trifluoro-2-(2-(3-(4-fluorophenyl)ureido)benzo[d]thiazol-6-yl)-2-(hydroxymethyl)propanamide (241)

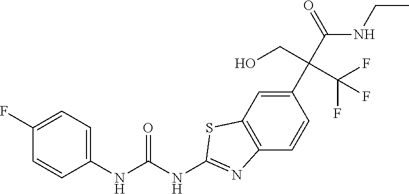 [SMILES: CCNC(=O)C(CO)(c1ccc2nc(NC(=O)Nc3ccc(F)cc3)sc2c1)C(F)(F)F]

To a solution of 3,3,3-trifluoro-2-(2-(3-(4-fluorophenyl)ureido)benzo[d]thiazol-6-yl)-2-(hydroxymethyl)propanoic acid (50.0 mg, 0.11 mmol) and ethyl amine hydrochloride (28.2 mg, 0.34 mmol) in DMF (1.1 mL) was added HOBt (51.8 mg, 0.34 mmol), DIPEA (159 μL, 0.9 mmol), and PyBOP (180 mg, 0.34 mmol) and the resulting mixture was stirred for 16 h. The reaction mixture was directly loaded onto C-18 column and purified by reverse phase HPLC (40% MeCN/Ammonium bicarbonate 10 mM buffer). The resulting product was dissolved in ethyl acetate (5 mL) and washed with 1 N HCl solution (5 mL×3), dried (Na₂SO₄), filtered and concentrated. The residue was dissolved in MeCN and water (1:1, 2 mL) and lyophilized to afford title compound (241) (4.4 mg, 8.1%) as a white solid. MS (ESI): mass calcd, for C₂₀H₁₈F₄N₄O₃S: 470.10, found: 471.2 [M+H]⁺, ¹H NMR (400 MHz, CD₃OD) δ ppm 7.88 (s, 1H), 7.79 (t, J=5.5 Hz, 1H), 7.67 (d, J=8.6 Hz, 1H), 7.49-7.56 (m, 2H), 7.42 (d, J=8.5 Hz, 1H), 7.05-7.14 (m, 2H), 4.37 (q, J=11.8 Hz, 2H), 3.11-3.18 (m, 2H), 1.12 (t, J=7.2 Hz, 3H).

### Example 245. (S)—N-Ethyl-3,3,3-trifluoro-2-(2-(3-(4-fluorophenyl)thioureido)benzo[d]thiazol-6-yl)-2-hydroxypropanamide (245)

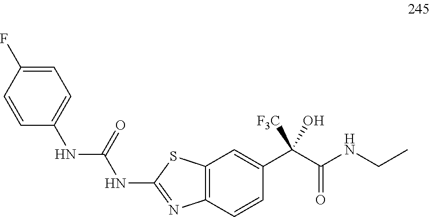 [SMILES: CCNC(=O)[C@@](O)(c1ccc2nc(NC(=O)Nc3ccc(F)cc3)sc2c1)C(F)(F)F]

### Synthetic Scheme:

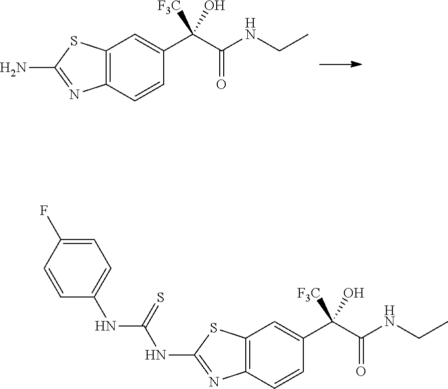 [SMILES: CCNC(=O)[C@@](O)(c1ccc2nc(N)sc2c1)C(F)(F)F ; CCNC(=O)[C@@](O)(c1ccc2nc(NC(=S)Nc3ccc(F)cc3)sc2c1)C(F)(F)F]

### (S)—N-Ethyl-3,3,3-trifluoro-2-(2-(3-(4-fluorophenyl)thioureido)benzo[d]thiazol-6-yl)-2-hydroxypropanamide (245)

To a solution of (S)-2-(2-aminobenzo[d]thiazol-6-yl)-N-ethyl-3,3,3-trifluoro-2-hydroxypropanamide (200 mg, 0.63 mmol) in DMF (2 mL) was added triethylamine (349 μL, 2.51 mmol) followed by 4-fluorophenyl isothiocyanate (506 mg, 3.14 mmol) and the solution was stirred at rt for 17 h. The reaction was directly purified by reverse phase HPLC (C18, 5-100% MeCN/Ammonium formate 10 mM buffer) to afford the title compound (245) (40 mg, 14%) as a yellow solid. MS (ESI): mass calcd, for C₁₀H₁₆F₄N₄O₂S₂: 472.07, found: 473.1 [M+H]⁺. ¹H NMR (400 MHz, DMSO-d6) δ ppm 8.22 (t, J=5.9 Hz, 1H), 8.17 (s, 1H), 8.02 (s, 1H), 7.77 (dd, J=8.8, 5.2 Hz, 2H), 7.61 (d, J=8.3 Hz, 1H), 7.49 (d, J=8.5 Hz, 1H), 7.13 (t, J=8.8 Hz, 2H), 3.08-3.17 (m, 3H), 1.00 (t, J=7.2 Hz, 3H).

### Example 246. (S)—N-Ethyl-3,3,3-trifluoro-2-(2-(3-(4-fluorophenyl) guanidino)benzo[d]thiazol-6-yl)-2-hydroxypropanamide (246)

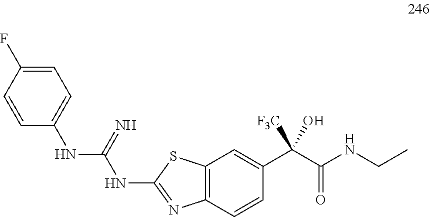 [SMILES: CCNC(=O)[C@@](O)(c1ccc2nc(NC(=N)Nc3ccc(F)cc3)sc2c1)C(F)(F)F]

### Synthetic Scheme:

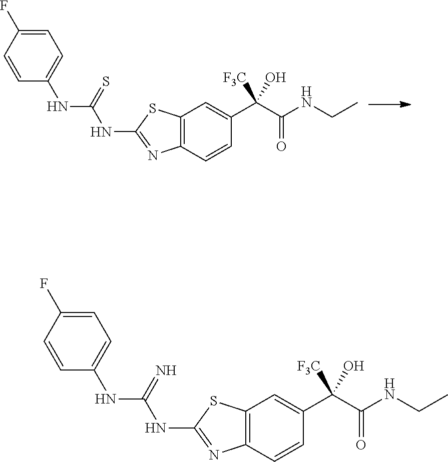 [SMILES: CCNC(=O)[C@@](O)(c1ccc2nc(NC(=S)Nc3ccc(F)cc3)sc2c1)C(F)(F)F ; CCNC(=O)[C@@](O)(c1ccc2nc(NC(=N)Nc3ccc(F)cc3)sc2c1)C(F)(F)F]

### (S)—N-Ethyl-3,3,3-trifluoro-2-(2-(3-(4-fluorophenyl) guanidino)benzo[d]thiazol-6-yl)-2-hydroxypropanamide (246)

To a stirred solution of (S)—N-ethyl-3,3,3-trifluoro-2-(2-(3-(4-fluorophenyl)thioureido)benzo[d]thiazol-6-yl)-2-hydroxypropanamide (30 mg, 64 μmol) in ACN (2 mL) was added dropwise a solution of 2-iodoxybenzoic acid (50.1 mg, 70 μmol) in aqueous ammonium hydroxide (30% v/v, 2 mL) at rt over a period of 10 min. After stirring for 1.5 h, the solvents were evaporated and the crude was purified by reverse phase HPLC (C18, 35-55% MeCN/Ammonium bicarbonate 10 mM buffer) to provide the title compound (246) (14 mg, 48%) as a white solid. MS (ESI): mass calcd, for C₁₉H₁₇F₄N₅O₂S: 455.10, found: 456.2 [M+H]⁺. ¹H NMR (400 MHz, DMSO-d6) δ ppm 9.42 (br s, 1H), 8.23 (br s, 2H), 7.98 (s, 1H), 7.79 (br s, 1H), 7.54-7.60 (m, 2H), 7.46-7.53 (m, 2H), 7.13-7.23 (m, 2H), 3.12 (dd, J=13.0, 6.9 Hz, 2H), 0.99 (t, J=7.2 Hz, 3H).

Examples 251 (used 3-methylthiophen-2-amine), Example 252 (used 4-methyl-1H-pyrazol-3-amine), Example 256 (used 5-aminoisothiazole-4-carbonitrile), Example 267 (used 5-amino-3-methylisothiazole-4-carbonitrile), Example 268 (4-chloro-3-methylisothiazol-5-amine, which was prepared from 3-methylisothiazol-5-amine hydrochloride using NCS in acetic acid at 50° C.), 272 (used 4-methylisoxazol-5-amine), Example 273 (used 4-chloroisothiazol-5-amine, which was prepared from 4-chloroisothiazole-5-carboxylic acid with DPPA, tBuOH at 100° C., followed by Boc deprotection) and Example 313 (used 1-methyl-1H-imidazol-5-amine) were synthesized in similar procedures as described in Example 27.

Examples 248, Example 249, Example 250, Example 261, Example 262, Example 263, Example 264, Example 265, 276 ((1-(trifluoromethyl)cyclobutyl) methanamine hydrochloride was used as the amine, which was prepared from 1-(trifluoromethyl)cyclobutane-1-carboxylic acid, reduced with borohydride to (1-(trifluoromethyl)cyclobutyl) methanol, followed by Mitsunobu conversion of the alcohol with phthalimide and then deprotection to form the amine), 279, 280, 291, 292, 293, 294, 295, 296, 304, 319, 320, 447, 455, and 456 were synthesized in similar procedures as described in Example 150. For Examples 248, 249, 261-265, 279, 280, 291-296, reaction was heated at 60° C. for 1 h after amine was added. For Examples 319 and 320, reaction was heated at 60° C. for 12 h after amine was added.

Example 253, Example 254, Example 257, Example 258, Example 266, Examples 270, 271, 274, 275, 287, 301, 314, and 318 were synthesized in similar procedures as described in Example 175.

### Example 255. N-Ethyl-3,3,3-trifluoro-2-(2-(3-(4-fluorophenyl)ureido)-4-(hydroxymethyl)benzo[d]thiazol-6-yl)-2-hydroxypropanamide (255)

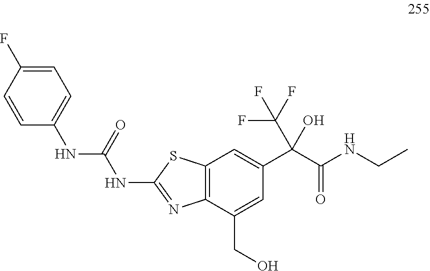 [SMILES: CCNC(=O)C(O)(c1cc(CO)c2nc(NC(=O)Nc3ccc(F)cc3)sc2c1)C(F)(F)F]

### Synthetic Scheme:

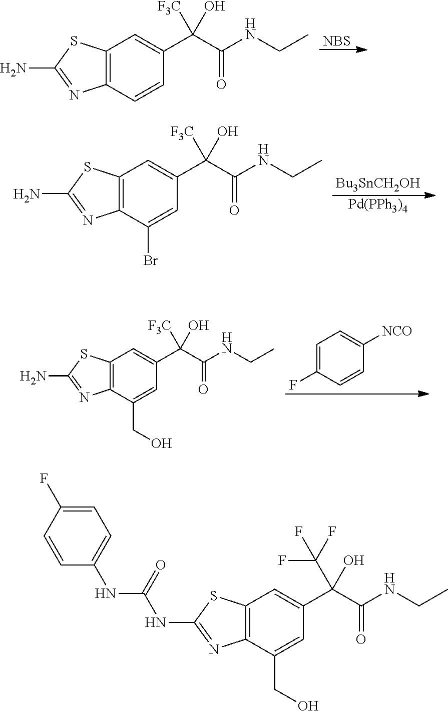

### 2-(2-Amino-4-bromo-1,3-benzothiazol-6-yl)-N-ethyl-3,3,3-trifluoro-2-hydroxy-propanamide

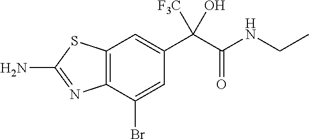 [SMILES: CCNC(=O)C(O)(c1cc(Br)c2nc(N)sc2c1)C(F)(F)F]

To a solution of 2-(2-amino-1,3-benzothiazol-6-yl)-N-ethyl-3,3,3-trifluoro-2-hydroxy-propanamide (1 g, 3.13 mmol) in ACN (10 mL) was added NBS (836.1 mg, 4.7 mmol). After stirring at 50° C. for 12 h. the reaction mixture was diluted with H₂O (10 mL) and extracted with EtOAc (10 mL×3). The combined organic layer was washed with brine, dried over anhydrous Na₂SO₄, filtered, and concentrated under reduced pressure. The resulting residue was purified by silica gel column chromatography (gradient elution, 0-50% EtOAc in PE) to afford the title compound (0.8 g, 64%) as a yellow solid. MS (ESI): mass calcd. for C₁₂H₁₁BrF₃N₃O₂S: 396.97, found: 398.1 [M+H]⁺.

### 2-(2-Amino-4-(hydroxymethyl)benzo[d]thiazol-6-yl)-N-ethyl-3,3,3-trifluoro-2-hydroxypropanamide

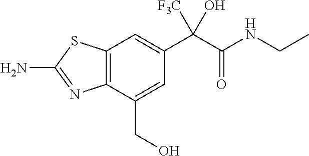 [SMILES: CCNC(=O)C(O)(c1cc(CO)c2nc(N)sc2c1)C(F)(F)F]

To a solution of 2-(2-amino-4-bromo-1,3-benzothiazol-6-yl)-N-ethyl-3,3,3-trifluoro-2-hydroxy-propanamide (0.25 g, 0.63 mmol) in dioxane (3 mL) was added Pd(PPh₃)₄ (72.6 mg, 0.063 mmol) and tributylstannylmethanol (302 mg, 0.94 mmol) at 25° C. under N₂. After heating at 80° C. for 16 h, the reaction mixture was concentrated under reduced pressure. The resulting residue was purified by reversed phase HPLC (gradient elution, 1-30% ACN in H₂O, with 10 mM NH₄HCO₃ as a modifier) to provide the title compound (0.01 g, 4.6%) as a white solid. MS (ESI): mass calcd, for C₁₃H₁₄F₃N₃O₃S: 349.07, found: 350.2 [M+H]⁺.

### N-Ethyl-3,3,3-trifluoro-2-(2-(3-(4-fluorophenyl)ureido)-4-(hydroxymethyl)benzo[d]thiazol-6-yl)-2-hydroxypropanamide (255)

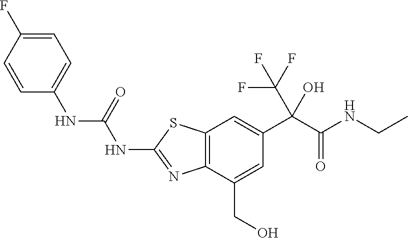 [SMILES: CCNC(=O)C(O)(c1cc(CO)c2nc(NC(=O)Nc3ccc(F)cc3)sc2c1)C(F)(F)F]

To a solution of 2-[2-amino-4-(hydroxymethyl)-1,3-benzothiazol-6-yl]-N-ethyl-3,3,3-trifluoro-2-hydroxy-propanamide (0.01 g, 29 μmol) in DMF (1 mL) was added 1-fluoro-4-isocyanato-benzene (3.9 μL, 34 μmol). It was then allowed to stir at 20° C. for 3 h. After completion, the reaction mixture was diluted with H₂O (2 mL) and extracted with EtOAc (2 mL×3). The combined organic layer was washed with brine, dried over anhydrous Na₂SO₄, filtered, and concentrated under reduced pressure. The resulting residue was purified by reversed phase HPLC (gradient elution, 15-40% ACN in H₂O, with 10 mM NH₄HCO; as a modifier) to provide the title compound (255) (1.5 mg, 10.8%, racemic) as a white solid. MS (ESI): mass calcd, for C₂₀H₁₈F₄N₄O₄S: 486.10, found: 487.1 [M+H]⁺. ¹H NMR (400 MHz, DMSO-d₆) δ ppm 9.05 (s, 1H), 8.15 (t, J=5.6 Hz, 1H), 8.01 (s, 1H), 7.78 (s, 1H), 7.70 (s, 1H), 7.51-7.55 (m, 3H), 7.16 (t, J=8.8 Hz, 2H), 5.28-5.40 (m, 1H), 4.87 (s, 2H), 3.09-3.16 (m, 2H), 1.00 (t, J=7.2 Hz, 3H).

### Example 269. (S)-2-(2-(3-(3,4-Dimethylisothiazol-5-yl)ureido)benzo[d]thiazol-6-yl)-N-ethyl-3,3,3-trifluoro-2-hydroxypropanamide (269)

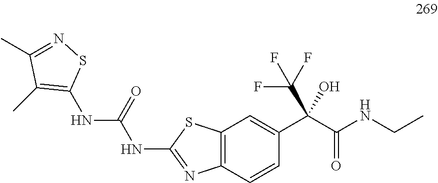 [SMILES: CCNC(=O)[C@@](O)(c1ccc2nc(NC(=O)Nc3snc(C)c3C)sc2c1)C(F)(F)F]

### Synthetic Scheme:

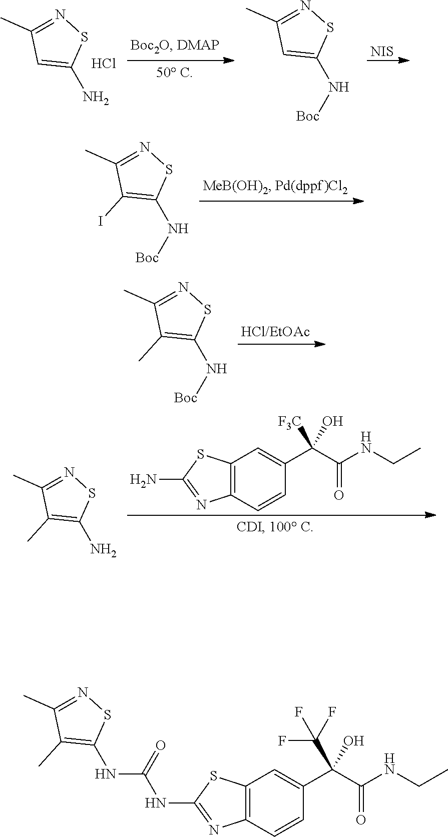

### tert-Butyl (3-methylisothiazol-5-yl)carbamate

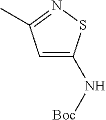

To a solution of 3-methylisothiazol-5-amine (2 g, 13.3 mmol, HCl salt) in dioxane (20 mL) was added TEA (2.69 g, 26.6 mmol), DMAP (2.43 g, 19.9 mmol) and Boc₂O (3.48 g, 15.9 mmol). After heating at 50° C. for 12 h, the reaction mixture was diluted with H₂O (20 mL) and extracted with EtOAc (20 mL×3). The combined organic layer was washed with brine, dried over Na₂SO₄, filtered and concentrated under reduced pressure. The residue was purified by silica gel column chromatography (Petroleum ether:Ethyl acetate=100:1 to 1:1) to provide the title compound (2 g, 70%) as a yellow solid. MS (ESI): mass calcd. for C₉H₁₄N₂O₂S: 214.08, found: 215.2 [M+H]⁺.

### tert-Butyl (4-iodo-3-methylisothiazol-5-yl)carbamate

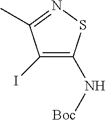

A solution of tert-butyl N-(3-methylisothiazol-5-yl)carbamate (1.94 g, 9.05 mmol) and NIS (2.44 g, 10.9 mmol) in AcOH (30 mL) was stirred for 12 h at 20° C. under N₂. The reaction was quenched with saturated NaHCO₃ (200 mL) and extracted with EtOAc (200 mL×3). The combined organic layer was washed with brine, dried over Na₂SO₄, filtered and concentrated under reduced pressure. The residue was purified by silica gel column chromatography (Petroleum ether:Ethyl acetate=50:1 to 20:1) to provide the title compound (2.84 g, 92.2%) as a yellow solid.

### tert-Butyl (3,4-dimethylisothiazol-5-yl)carbamate

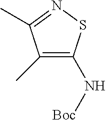

To a solution of tert-butyl N-(4-iodo-3-methyl-isothiazol-5-yl)carbamate (2.62 g, 7.7 mmol) and methylboronic acid (922 mg, 15.4 mmol) in dioxane (30 mL) and H₂O) (3 mL) was added K₂CO₃ (3.19 g, 23.1 mmol) and Pd(dppf)Cl₂ (563.5 mg, 0.77 mmol) at 20° C. under N₂. The mixture was heated to 90° C. and stirred for 12 h. The reaction mixture was filtered through a pad of celite. The filtrate was concentrated under reduced pressure. The residue was purified by reverse phase HPLC (H₂O (10 mM NH₄HCO₃)-ACN], gradient 22-52% elution) to give the title compound (613 mg, 34.9%) as a yellow solid. MS (ESI): mass calcd, for C₁₀H₁₆N₂O₂S: 228.09, found: 229.1 [M+H]⁺.

### 3,4-Dimethylisothiazol-S-amine

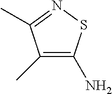

A solution of tert-butyl N-(3,4-dimethylisothiazol-5-yl)carbamate (613 mg, 2.68 mmol) in EtOAc (5 mL) and HCV/EtOAc (20 mL) was stirred for 12 h at 20° C. under N₂. The reaction mixture was concentrated under reduced pressure to provide the crude title compound (0.32 g, HCl salt) as a white solid. MS (ESI): mass calcd, for C₅H₈N₂S: 128.04, found: 129.2 [M+H]⁺.

### (S)-2-(2-(3-(3,4-Dimethylisothiazol-5-yl)ureido)benzo[d]thiazol-6-yl)-N-ethyl-3,3,3-trifluoro-2-hydroxypropanamide (269)

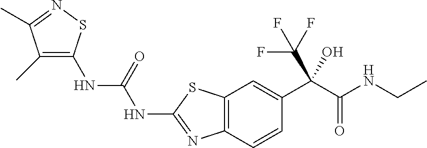 [SMILES: CCNC(=O)[C@@](O)(c1ccc2nc(NC(=O)Nc3snc(C)c3C)sc2c1)C(F)(F)F]

To a mixture of 3,4-dimethylisothiazol-5-amine HCl salt (0.1 g, crude) and (S)-2-(2-aminobenzo[d]thiazol-6-yl)-N-ethyl-3,3,3-trifluoro-2-hydroxypropanamide (250 mg, 0.78 mmol) in DMF (5 mL) was added CDI (253 mg, 1.56 mmol) at 0° C. under N₂. The mixture was heated to 100° C. and stirred for 12 h. The reaction was diluted with H₂O (15 mL) and extracted with EtOAc (20 mL×3). The combined organic layer was washed with brine, dried over Na₂SO₄, filtered and concentrated under reduced pressure. The residue was purified by reverse phase HPLC (H₂O (0.2% FA)-ACN, gradient 30-60% elution), to provide the title compound (105 mg, 28%) as a white solid. MS (ESI): mass calcd, for C₁₈H₁₈F₃N₅O₃S₂: 473.08, found: 474.1 [M+H]⁺. ¹H NMR (400 MHz, DMSO-d6) δ ppm 11.21 (s, 1H), 9.81-10.39 (br s, 1H), 8.22 (t, J=6.0 Hz, 1H), 8.14-8.19 (m, 1H), 7.74-7.87 (m, 1H), 7.67 (s, 2H), 3.09-3.31 (m, 2H), 2.26 (s, 3H), 2.12 (s, 3H), 0.99 (t, J=7.2 Hz, 3H).

### Example 278. N-Ethyl-3,3,3-trifluoro-2-(2-(3-(4-fluorophenyl)ureido)-4-(hydroxymethyl)benzo[d]thiazol-6-yl)-2-hydroxypropanamide (278)

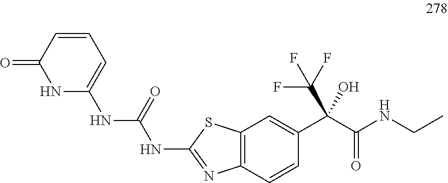 [SMILES: CCNC(=O)[C@@](O)(c1ccc2nc(NC(=O)Nc3cccc(=O)[nH]3)sc2c1)C(F)(F)F]

### Synthetic Scheme:

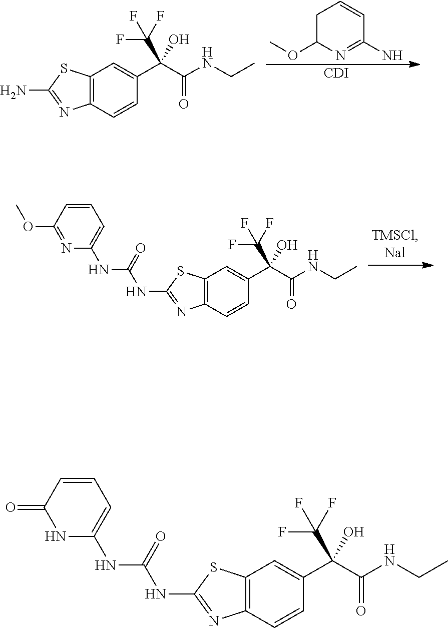

### (2S)—N-Ethyl-3,3,3-trifluoro-2-hydroxy-2-[2-[(6-methoxy-2-pyridyl) carbamoylamino]-1,3-benzothiazol-6-yl]propanamide

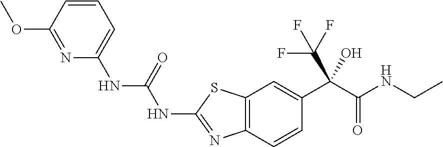 [SMILES: CCNC(=O)[C@@](O)(c1ccc2nc(NC(=O)Nc3cccc(OC)n3)sc2c1)C(F)(F)F]

To a solution of (2S)-2-(2-amino-1,3-benzothiazol-6-yl)-N-ethyl-3,3,3-trifluoro-2-hydroxy-propanamide (0.25 g, 0.78 mmol) in DMF (2 mL) was added CDI (254 mg, 1.57 mmol) and 6-methoxypyridin-2-amine (117 mg, 0.94 mmol), After heating at 100° C. for 12 h, the reaction mixture was poured into H₂O (10 mL). The aqueous layer was extracted with EtOAc (10 mL×3). The combined organic layer was washed with brine, dried over Na₂SO₄, filtered, concentrated, and purified by silica gel column chromatography (gradient elution, 0-33% EtOAc in PE) to afford the title compound (0.12 g, 32.7%) as a white solid. MS (ESI): mass calcd, for C₁₉H₁₈F₃N₅O₄S: 469.10, found: 470.0 [M+H]⁺.

### N-Ethyl-3,3,3-trifluoro-2-(2-(3-(4-fluorophenyl)ureido)-4-(hydroxymethyl)benzo[d]thiazol-6-yl)-2-hydroxypropanamide (278)

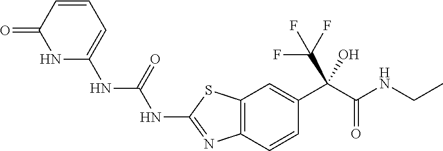 [SMILES: CCNC(=O)[C@@](O)(c1ccc2nc(NC(=O)Nc3cccc(=O)[nH]3)sc2c1)C(F)(F)F]

TMSCl (122 μL, 0.96 mmol) was added to a solution of NaI (144 mg, 0.96 mmol) in ACN (1 mL) dropwise at rt. After stirring for 15 minutes, it was cooled to 0° C. and a solution of (2S)—N-ethyl-3,3,3-trifluoro-2-hydroxy-2-[2-[(6-methoxy-2-pyridyl) carbamoylamino]-1,3-benzothiazol-6-yl]propanamide (0.1 g, 0.21 mmol) in ACN (1 mL) was added dropwise. It was then allowed to stir at rt for 10 minutes and further heated at 70° C. for 1.5 h. Upon completion, the reaction mixture was cooled to rt and quenched with H₂O (10 mL) and 20% aqueous Na₂S₂O₃ (20 mL) and extracted with EtOAc (20 mL×3). The combined organic layer was dried over Na₂SO₄, filtered, concentrated, and purified by reverse phase HPLC (gradient elution, 6-36% ACN in H₂O, with 10 mM NH₄HCO₃ as a modifier) to afford the title compound (278) (68 mg, 70%) as a white solid. MS (ESI): mass calcd, for C₁₈H₁₆F₃N₅O₄S: 455.09, found: 456.1 [M+H]⁺. ¹H NMR (400 MHz, DMSO-d₆) δ ppm 11.99 (s, 1H), 9.66 (s, 1H), 8.18-8.24 (m, 2H), 7.55-7.78 (m, 4H), 6.94 (s, 1H), 6.29 (s, 1H), 3.18-3.29 (m, 2H), 1.00 (t, J=7.2 Hz, 3H).

### Example 281. N-Ethyl-3-(2-(3-(4-fluorophenyl)ureido)benzo[d]thiazol-6-yl)azetidine-3-carboxamide (281)

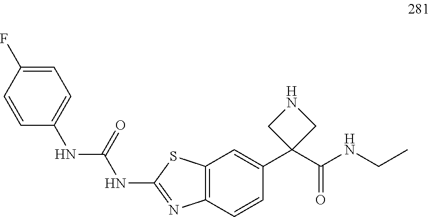 [SMILES: CCNC(=O)C1(c2ccc3nc(NC(=O)Nc4ccc(F)cc4)sc3c2)CNC1]

### Synthetic Scheme:

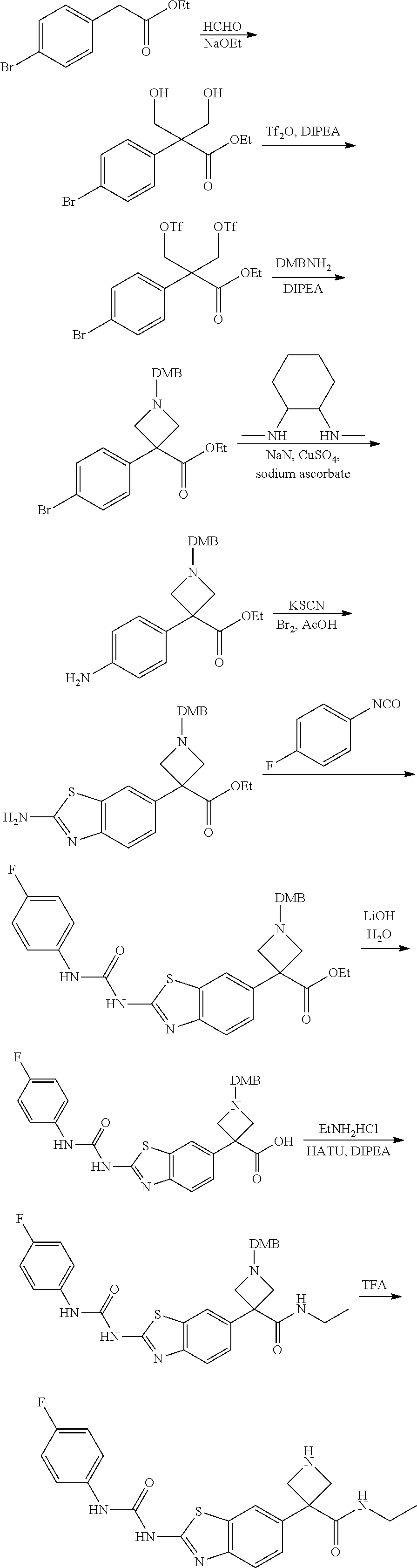

### Ethyl 2-(4-bromophenyl)-3-hydroxy-2-(hydroxymethyl)propanoate

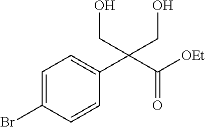 [SMILES: CCOC(=O)C(CO)(CO)c1ccc(Br)cc1]

A mixture of ethyl 2-(4-bromophenyl)acetate (10 g, 41.1 mmol), HCHO (9.19 mL, 123.4 mmol) and NaOEt (560 mg, 8.23 mmol) in DMF (200 mL) was degassed and purged with N2× 3. It was then allowed to stir at 20° C. for 12 h under N₂. Upon completion, the reaction was partitioned between EtOAc (600 mL) and H₂O (600 mL) and extracted with EtOAc (600 mL×2). The combined organic layer was washed with brine, dried over anhydrous Na₂SO₄, filtered, concentrated, and purified by silica gel column chromatography (gradient elution, 0-50% EtOAc in PE) to provide the title compound (15 g, 30%) as a white solid. ¹H NMR (400 MHz, DMSO-d6) δ ppm 7.47-7.51 (m, 2H), 7.12-7.15 (m, 2H), 4.78 (t, J=7.2 Hz, 2H), 4.03-4.07 (m, 2H), 3.89-3.96 (m, 4H), 1.11 (t, J=7.2 Hz, 3H).

### Ethyl 2-(4-bromophenyl)-3-(trifluoromethylsulfonyloxy)-2-(trifluoromethylsulfonyloxy methyl)propanoate

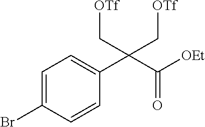 [SMILES: CCOC(=O)C(COS(=O)(=O)C(F)(F)F)(COS(=O)(=O)C(F)(F)F)c1ccc(Br)cc1]

To a solution of the ethyl 2-(4-bromophenyl)-3-hydroxy-2-(hydroxymethyl)propanoate (5 g, 16.5 mmol) in ACN (80 mL) at −20° C. was slowly added Tf₂O (5.71 mL, 34.6 mmol) over 15 minutes, followed by the addition of DIPEA (7.2 mL, 41.2 mmol) over another 15 minutes. After stirring at −20° C. for 1 h, the reaction was concentrated under reduced pressure to afford the crude title compound (10 g, 53.4%) as a brown oil, which was used in the next step directly without further purification.

### Ethyl 3-(4-bromophenyl)-1-(3,4-dimethylbenzyl)azetidine-3-carboxylate

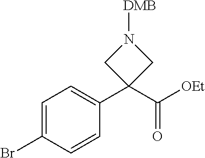

To a solution of ethyl 2-(4-bromophenyl)-3-(trifluoromethylsulfonyloxy)-2-(trifluoromethylsulfonyloxymethyl)propanoate (10 g, 17.6 mmol) in ACN (10 mL) was added (2,4-dimethoxyphenyl) methanamine (2.65 mL, 17.6 mmol). After 5 minutes, DIPEA (7.68 mL, 44.1 mmol) was added. After heating at 70° C. for 1 b, the reaction solvent was removed under reduced pressure. The resulting residue was purified by silica gel column chromatography (gradient elution. 0-100% EtOAc in PE) to afford the title compound (1.4 g, 18.3%) as a yellow oil. MS (ESI): mass calcd, for C₂₁H₂₄BrNO₄: 433.09, found: 434.1 [M+H]⁺.

### Ethyl 1-(3,4-dimethylbenzyl)-3-(2-(3-(4-fluorophenyl)ureido)benzo[d]thiazol-6-yl)azetidine-3-carboxylate

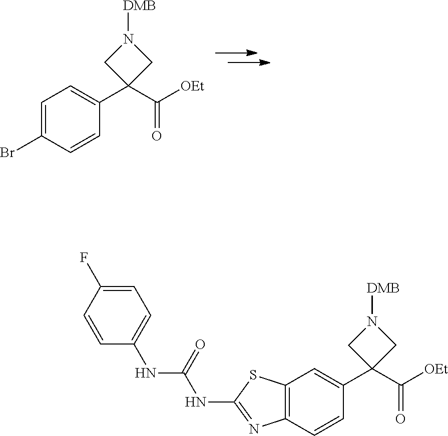

The title compound was synthesized from ethyl 3-(4-bromophenyl)-1-(3,4-dimethylbenzyl)azetidine-3-carboxylate in similar procedures as described in Example 1. MS (ESI): mass calcd, for C₂₉H₂₉FN₄O₅S: 564.18, found: 565.2 [M+H]⁺.

### 1-(3,4-Dimethylbenzyl)-N-ethyl-3-(2-(3-(4-fluorophenyl)ureido)benzo[d]thiazol-6-yl)azetidine-3-carboxamide

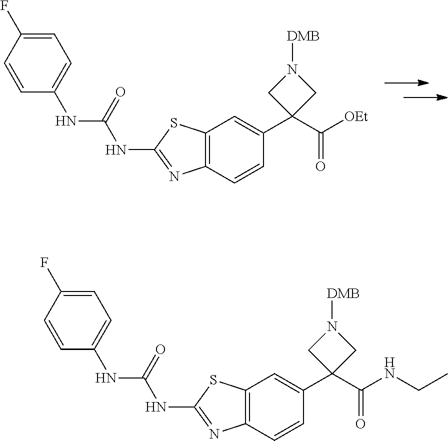

The title compound was synthesized from ethyl 1-(3,4-dimethylbenzyl)-3-(2-(3-(4-fluorophenyl)ureido)benzo[d]thiazol-6-yl)azetidine-3-carboxylate in similar procedures as described in Example 38. MS (ESI): mass calcd, for C₂₉H₃₀FN₅O₄S: 563.20, found: 564.2 [M+H]⁺.

### N-Ethyl-3-(2-(3-(4-fluorophenyl)ureido)benzo[d]thiazol-6-yl)azetidine-3-carboxamide (281)

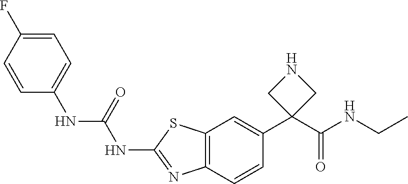 [SMILES: CCNC(=O)C1(c2ccc3nc(NC(=O)Nc4ccc(F)cc4)sc3c2)CNC1]

A mixture of 1-(3,4-dimethylbenzyl)-N-ethyl-3-(2-(3-(4-fluorophenyl)ureido)benzo[d]thiazol-6-yl)azetidine-3-carboxamide (60 mg, 0.11 mmol) and 1-chloroethyl carbonochloridate (15.2 mg, 0.11 mmol) in DCM (1 mL) was degassed and purged with N₂×3. After stirring at 25° C. under N₂ for 2 h, the reaction was quenched with aqueous NaHCO₃ (5 mL). The resulting mixture was further diluted with H₂O (5 mL) and extracted with EtOAc (10 mL×3). The combined organic layer was dried over Na₂SO₄, filtered, concentrated, and purified by reverse phase HPLC (gradient elution, 5-40% ACN in H₂O, with 0.2% FA as a modifier) to afford the title compound (281) (2.1 mg, 4.8%) as a white solid. MS (ESI): mass calcd, for C₂₀H₂₀FN₅O₂S: 413.13, found: 414.1 [M+H]⁺. ¹H NMR (400 MHz, DMSO-d₆) δ ppm 10.08 (s, 1H), 8.41 (s, 1H), 8.85 (d, J=5.6 Hz, 2H), 7.55-7.60 (m, 3H), 7.28-7.30 (m, 1H), 7.15 (t, J=8.8 Hz, 2H), 4.19 (d, J=8.4 Hz, 2H), 3.92 (d, J=8.8 Hz, 2H), 3.03-3.10 (m, 2H), 0.97 (t, J=7.2 Hz, 3H).

### Example 282. 2-(4-Cyano-2-(3-(4-fluorophenyl)ureido)benzo[d]thiazol-6-yl)-N-ethyl-3,3,3-trifluoro-2-hydroxypropanamide (282)

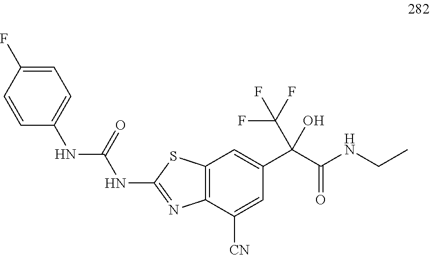 [SMILES: CCNC(=O)C(O)(c1cc(C#N)c2nc(NC(=O)Nc3ccc(F)cc3)sc2c1)C(F)(F)F]

### Synthetic Scheme:

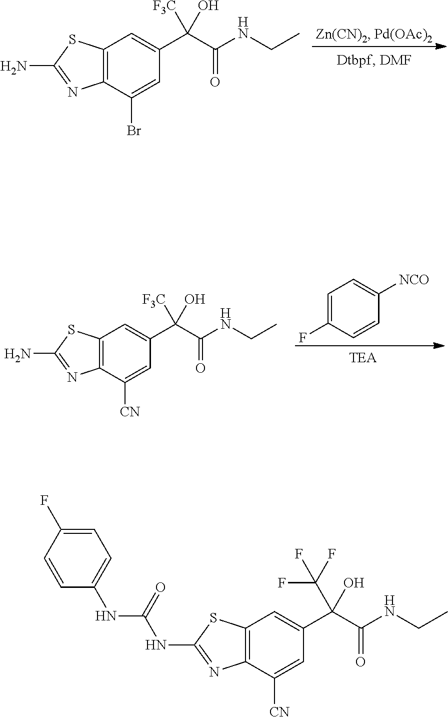

### 2-(2-Amino-4-cyanobenzo[d]thiazol-6-yl)-N-ethyl-3,3,3-trifluoro-2-hydroxypropanamide

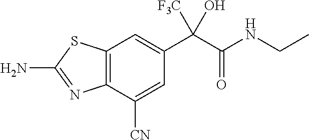 [SMILES: CCNC(=O)C(O)(c1cc(C#N)c2nc(N)sc2c1)C(F)(F)F]

To a solution of 2-(2-amino-4-bromo-1,3-benzothiazol-6-yl)-N-ethyl-3,3,3-trifluoro-2-hydroxy-propanamide (described in Example 259, 100 mg, 0.25 mmol) in DMA (2 mL) was added Zn(CN)₂ (29 mg, 0.25 mmol), Pd(OAc)₂ (5.64 mg, 0.025 mmol) and ditert-butyl(cyclopentyl)phosphane;iron (11.91 mg, 0.025 mmol) under N₂. It was then allowed to stir at 120° C. for 12 h. Upon completion, the reaction was quenched with sat. Na₂CO₃ solution (5 mL) and extracted with EtOAc (5 mL×3). The combined organic layer was washed with brine, dried over Na₂SO₄, filtered, concentrated. The crude was purified by reverse phase HPLC (gradient elution, 1-30% ACN in H₂O, with 10 mM NH₄HCO₃ as a modifier) to provide the title compound (34 mg, 39.3%) as a white solid. MS (ESI): mass calcd, for C₁₃H₁₁F₃N₄O₂S: 344.06, found: 345.0 [M+H]⁺.

### 2-(4-Cyano-2-(3-(4-fluorophenyl)ureido)benzo[d]thiazol-6-yl)-N-ethyl-3,3,3-trifluoro-2-hydroxypropanamide (282)

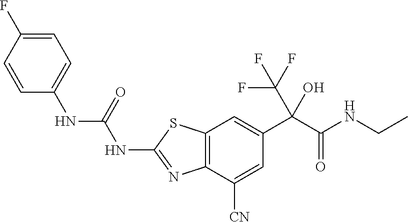 [SMILES: CCNC(=O)C(O)(c1cc(C#N)c2nc(NC(=O)Nc3ccc(F)cc3)sc2c1)C(F)(F)F]

To a solution of 2-(2-amino-4-cyanobenzo[d]thiazol-6-yl)-N-ethyl-3,3,3-trifluoro-2-hydroxypropanamide (50 mg, 0.15 mmol) in THF (1 mL) was added 1-fluoro-4-isocyanato-benzene (24 μL, 0.22 mmol) and TEA (40 μL, 0.29 mmol). Then it was allowed to stir at 60° C. for 12 h. After completion, the reaction mixture was concentrated under reduced pressure. The resulting residue was purified by normal phase HPLC to afford the title compound (282) (1.7 mg, 2.4%, racemic) as a white solid. MS (ESI): mass calcd, for C₂₀H₁₅F₄N₅O₃S: 481.08, found: 482.1 [M+H]⁺. ¹H NMR (400 MHz, DMSO-d6) δ ppm 11.66 (s, 1H), 9.11 (s, 1H), 8.55 (s, 1H), 8.31-8.39 (m, 1H), 8.09 (s, 1H), 8.02 (s, 1H), 7.49-7.59 (m, 2H), 7.15-7.23 (m, 2H), 3.06-3.20 (m, 2H), 0.99 (t, J=7.2 Hz, 3H).

### Example 288. (S)—N-Cyclopropyl-3,3,3-trifluoro-2-(2-(3-(4-fluoroisothiazol-5-yl)ureido)benzo[d]thiazol-6-yl)-2-hydroxypropanamide (288)

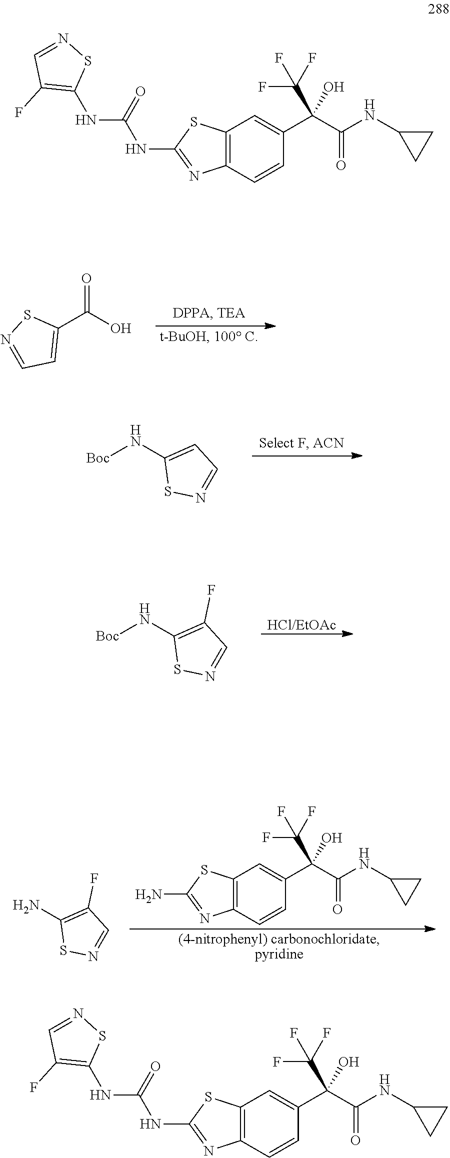

### tert-Butyl N-isothiazol-5-ylcarbamate

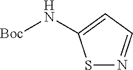 [SMILES: CC(C)(C)OC(=O)Nc1ccns1]

To a solution of isothiazole-5-carboxylic acid (1 g, 7.74 mmol) in t-BuOH (10 mL) was added DPPA (2.13 g, 7.74 mmol) and TEA (784 mg, 7.74 mmol). After heating at 100° C. for 12 h, the reaction was poured into H₂O (30 mL) and extracted with EtOAc (20 mL×3). The combined organic layer was washed with brine, dried with anhydrous Na₂SO₄, filtered and concentrated under reduced pressure. The residue was purified by silica gel column chromatography (Petroleum ether:Ethyl acetate=30:1 to 8:1) to provide the title compound (1.2 g, 77%) as a yellow solid. MS (ESI): mass calcd, for C₈H₁₂N₂O₂S: 200.06, found: 201.1 [M+H]⁺.

### tert-Butyl N-(4-fluoroisothiazol-5-yl)carbamate

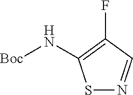 [SMILES: CC(C)(C)OC(=O)Nc1sncc1F]

To a solution of tert-butyl N-isothiazol-5-ylcarbamate (2 g, 9.99 mmol) in ACN (15 mL) was added Selectfluor (Select F) (5.31 g, 15 mmol). The reaction was stirred at 60° C. for 12 h and filtered. The filtrate was concentrated under reduced pressure. The residue was purified by silica gel column chromatography (Petroleum ether:Ethyl acetate=30:1 to 8:1) to provide the title compound (0.7 g, 32%) as a yellow solid. MS (ESI): mass calcd. for C₈H₁₁FN₂O₂S: 218.05, found: 219.0 [M+H]⁺.

### 4-Fluoroisothiazol-5-amine

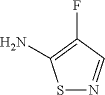

To a solution of tert-butyl N-(4-fluoroisothiazol-5-yl)carbamate (0.7 g, 3.21 mmol) in EtOAc (6 mL) was added HCV/EtOAc (4 M, 4.01 mL) at 0° C. The reaction was stirred at 25° C. for 12 h. Then it was filtered to provide the crude title compound (400 mg, HCl salt) as a yellow solid, which was used in the next step directly without further purification. MS (ESI): mass calcd, for C₃H₃FN₂S: 118.00, found: 119.1 [M+H]⁺

### (2S)—N-Cyclopropyl-3,3,3-trifluoro-2-[2-[(4-fluoroisothiazol-5-yl) carbamoylamino]-1,3-benzothiazol-6-yl]-2-hydroxy-propanamide (288)

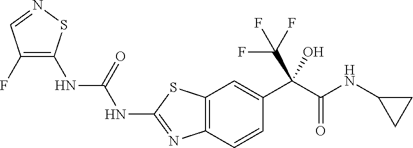 [SMILES: O=C(Nc1nc2ccc([C@](O)(C(=O)NC3CC3)C(F)(F)F)cc2s1)Nc1sncc1F]

To a solution of (2S)-2-(2-amino-1,3-benzothiazol-6-yl)-N-cyclopropyl-3,3,3-trifluoro-2-hydroxy-propanamide (20 mg, 60 μmol) in THF (2 mL) was added pyridine (95.5 mg, 1.21 mmol) and (4-nitrophenyl) carbonochloridate (121.7 mg, 0.6 mmol). After stirring at 25° C. for 1 h. 4-fluoroisothiazol-5-amine (18.7 mg, HCl salt) was added, and the reaction was stirred at 60° C. for 12 h. The reaction was quenched with H₂O (10 mL) and extracted with EtOAc (10 mL×3). The combined organic layer was washed with brine, dried over Na₂SO₄, filtered and concentrated under reduced pressure. The residue was purified by reverse phase HPLC (H₂O (0.2% FA)-ACN, gradient 20-50% elution) to provide the title compound (288) (6.4 mg, 22.3%) as a white solid. MS (ESI): mass calcd, for C₁₇H₁₃F₄N₅O₃S₂: 475.04, found: 476.1 [M+H]⁺. ¹H NMR (400 MHz, DMSO-d6) δ ppm 8.35-8.36 (m, 1H), 8.13-8.35 (m, 2H), 7.60-7.66 (m, 2H), 2.50-2.75 (m, 1H), 0.61-0.64 (m, 4H).

### Example 311. (S)—N-Ethyl-3,3,3-trifluoro-2-hydroxy-2-(2-(3-(4-(trifluoromethyl) isothiazol-S-yl)ureido)benzo[d]thiazol-6-yl)propenamide (311)

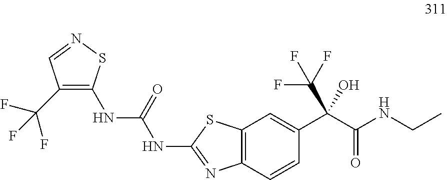 [SMILES: CCNC(=O)[C@@](O)(c1ccc2nc(NC(=O)Nc3sncc3C(F)(F)F)sc2c1)C(F)(F)F]

### Synthetic Scheme:

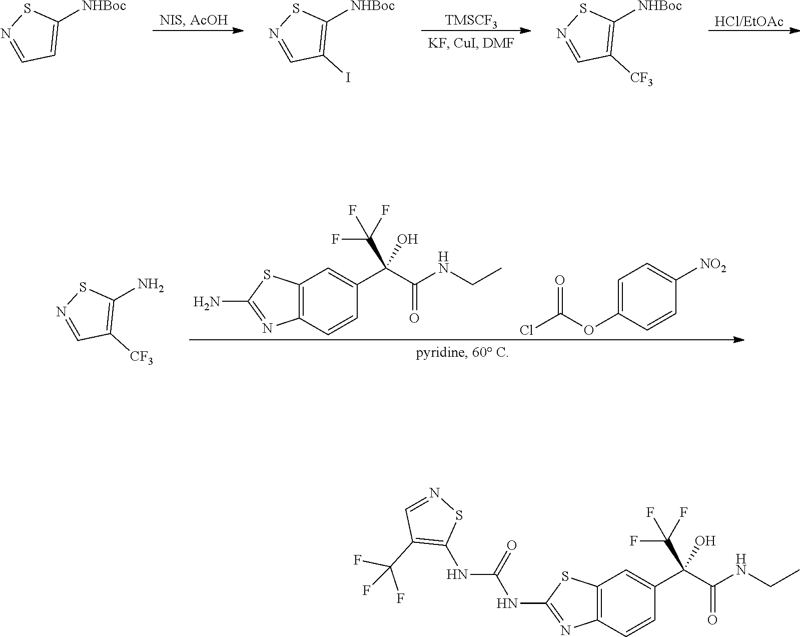

### tert-Butyl N-(4-iodoisothiazol-5-yl)carbamate

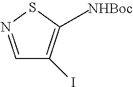

To a solution of tert-butyl N-isothiazol-5-ylcarbamate (7 g, 35 mmol) in HOAc (70 mL) was added NIS (9.44 g, 42 mmol). After stirring at 25° C. for 12 h, the reaction was concentrated under reduced pressure. Saturated NaHCO₃ was added to the crude residue to adjust to pH=7 and the reaction was extracted with ethyl acetate (300 mL×3). The combined organic phase was washed with brine, dried with anhydrous Na₂SO₄, filtered and concentrated under reduced pressure. The residue was purified by silica gel column chromatography (Petroleum ether:Ethyl acetate=30:1 to 20:1) to provide the title compound (10.4 g, 91%) as a yellow solid. MS (ESI): mass calcd, for C₈H₁₁IN₂O₂S: 325.96, found: 327.0 [M+H]⁺.

### tert-butyl N-[4-(trifluoromethyl) isothiazol-5-yl]carbamate

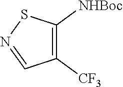 [SMILES: CC(C)(C)OC(=O)Nc1sncc1C(F)(F)F]

To a solution of tert-butyl N-(4-iodoisothiazol-S-yl)carbamate (0.5 g, 1.53 mmol) and trimethyl(trifluoromethyl) silane (1.09 g, 7.67 mmol) in DMF (10 mL) in a sealed tube was added iodocopper (1.46 g, 7.67 mmol) and KF (445 mg, 7.67 mmol) under N₂. After heating at 100° C. for 12 h, the reaction was filtered and concentrated under reduced pressure. The residue was purified by silica gel column chromatography (Petroleum ether:Ethyl acetate=1:0 to 10:1) to provide the title compound (0.3 g, 36.5%) as a yellow solid. MS (ESI): mass calcd, for C₉H₁₁F₃N₂O₂S: 268.05, found: 213.0 [M−56+H]⁺.

### 4. (Trifluoromethyl) isothiazol-S-amine

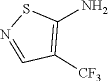

Two batches were carried out in parallel. To a mixture of tert-butyl N-[4-(trifluoromethyl) isothiazol-5-yl]carbamate (150 mg, 0.56 mmol) in EtOAc (0.5 mL) was added HCl/EtOAc (4 M, 15 mL) under N₂ at 0° C. After stirring at 25° C. for 1 h under N₂, the 2 batches were combined for workup. The reaction was concentrated under reduced pressure to provide the title compound (220 mg, HCl salt) as a yellow oil, which was used directly in the next step without further purification. MS (ESI): mass calcd, for C₄H₃F₃N₂S: 168.00, found: 169.0 [M+H]⁺.

### (S)—N-Ethyl-3,3,3-trifluoro-2-hydroxy-2-(2-(3-(4-(trifluoromethyl) isothiazol-5-yl)ureido)benzo[d]thiazol-6-yl)propenamide (311)

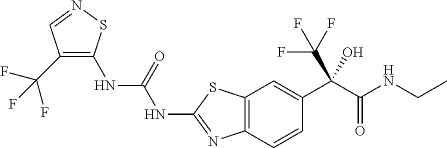 [SMILES: CCNC(=O)[C@@](O)(c1ccc2nc(NC(=O)Nc3sncc3C(F)(F)F)sc2c1)C(F)(F)F]

To a solution of (2S)-2-(2-amino-1,3-benzothiazol-6-yl)-N-ethyl-3,3,3-trifluoro-2-hydroxy-propanamide (0.05 g, 0.16 mmol) in THE (1 mL) was added pyridine (247.7 mg, 3.13 mmol) and (4-nitrophenyl) carbonochloridate (315.6 mg, 1.57 mmol). The reaction was stirred at 25° C. for 1 h. 4-(Trifluoromethyl) isothiazol-5-amine (38.45 mg, crude, HCl salt) was added and the reaction was heated at 60° C. for 12 h. The reaction was concentrated under reduced pressure. The residue was purified by reverse phase HPLC (H₂O (0.2% FA)-ACN; gradient: 20-60%) to provide the title compound (311) (0.016 g, 16%) as a yellow solid. MS (ESI): mass calcd, for C₁₇H₁₃F₆N₅O₃S₂: 513.04, found: 514.0 [M+H]⁺. ¹H NMR (400 MHz, DMSO-d6) δ ppm 11.67 (s, 1H), 10.89 (s, 1H), 8.52-8.56 (m, 1H), 8.21-8.24 (m, 2H), 7.83 (s, 1H), 7.72 (s, 2H), 3.09-3.16 (m, 2H), 0.99 (t, J=7.2 Hz, 3H).

### Example 333 and 344. 1-(6-((S)-3-(S)-2-Cyano-2-methylazetidin-1-yl)-1,1,1-trifluoro-2-hydroxy-3-oxopropan-2-yl)benzo[d]thiazol-2-yl)-3-(4-fluorophenyl)urea (333) and 1-(6-((S)-3-((R)-2-Cyano-2-methylazetidin-1-yl)-1,1,1-trifluoro-2-hydroxy-3-oxopropan-2-yl)benzo[d]thiazol-2-yl)-3-(4-fluorophenyl)urea (344)

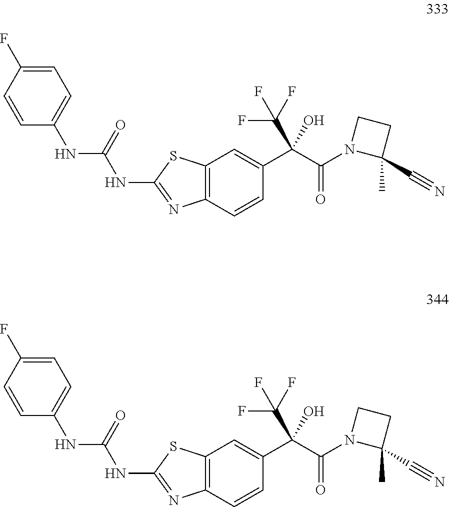 [SMILES: C[C@@]1(C#N)CCN1C(=O)[C@@](O)(c1ccc2nc(NC(=O)Nc3ccc(F)cc3)sc2c1)C(F)(F)F ; C[C@]1(C#N)CCN1C(=O)[C@@](O)(c1ccc2nc(NC(=O)Nc3ccc(F)cc3)sc2c1)C(F)(F)F]

### Synthetic Scheme:

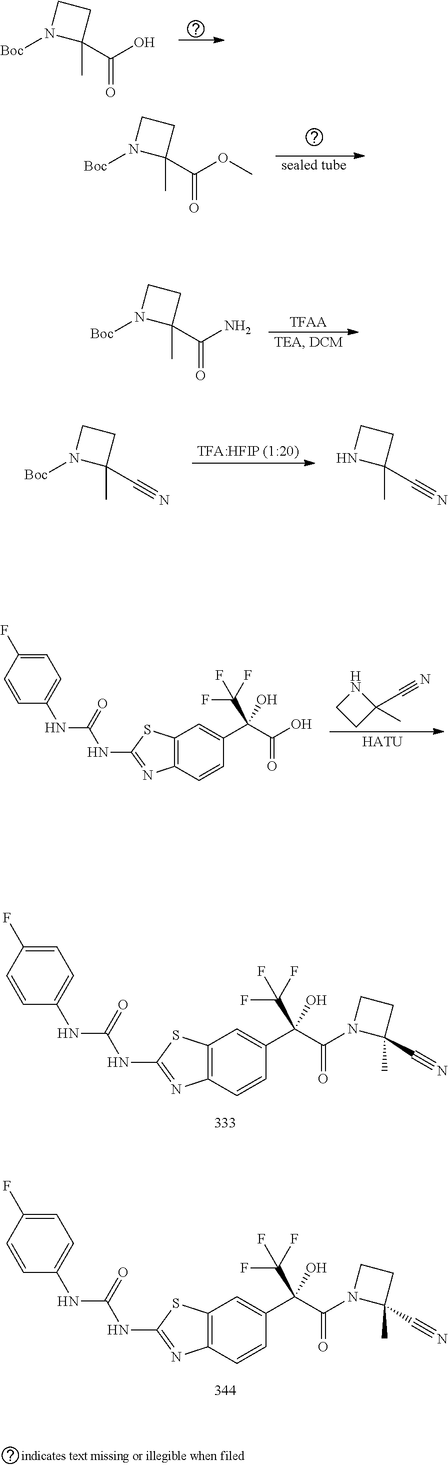

### 1-(tert-Butyl) 2-methyl 2-methylazetidine-1,2-dicarboxylate

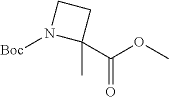 [SMILES: COC(=O)C1(C)CCN1C(=O)OC(C)(C)C]

To a solution of 1-tert-butoxy carbonyl-2-methyl-azetidine-2-carboxylic acid (1 g, 4.65 mmol) in DMF (5 mL) was added MeI (725 mg, 5.11 mmol) and K₂CO₃ (321 mg, 2.32 mmol). After stirring at 25° C. for 12 h, the reaction was diluted with H₂O (10 mL) and extracted with EtOAc (10 mL×3). The combined organic layer was washed with brine, dried over Na₂SO₄, filtered and concentrated under reduced pressure. The residue was purified by silica gel column chromatography (Petroleum ether:Ethyl acetate=10:1 to 1:1) to provide the title compound (1 g, 94%) as a white solid. MS (ESI): mass calcd, for C₁₁H₁₉NO₄: 229.13, found: 174.1 [M−56+H]⁺.

### tert-Butyl 2-carbamoyl-2-methyl-azetidine-1-carboxylate

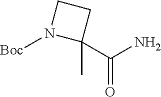

A solution of 1-(tert-butyl) 2-methyl 2-methylazetidine-1,2-dicarboxylate (1 g, 4.36 mmol) in MeOH (1 mL) was added NH₃/MeOH (7 M, 5.0 mL). The reaction was stirred under 15 Psi at 100° C. for 24 h in a sealed tube. The reaction mixture was concentrated under reduced pressure to provide the title compound (0.8 g) as a white solid, which was used directly in the next step without further purification. MS (ESI): mass calcd, for C₁₀H₁₈N₂O₃: 214.13, found: 115.3 [M−100+H]⁺.

### tert-Butyl 2-cyano-2-methyl-azetidine-1-carboxylate

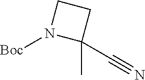

To a solution of tert-butyl 2-carbamoyl-2-methyl-azetidine-1-carboxylate (0.28 g, 1.31 mmol) in DCM (2 mL) was added TEA (397 mg, 3.92 mmol) and TFAA (549 mg, 2.61 mmol) at 25° C. After stirring at 25° C. for 3 h, the reaction was diluted with H₂O (5 mL) and extracted with EtOAc (5 mL×3). The combined organic layer was washed with brine, dried over Na₂SO₄, filtered and concentrated under reduced pressure. The residue was purified by prep-TLC (SiO₂, Petroleum ether:Ethyl acetate=1:1) to provide the title compound (50 mg, 19.5%) as a white solid.

### 2-Methylazetidine-2-carbonitrile

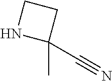

A solution of tert-butyl 2-cyano-2-methyl-azetidine-1-carboxylate (0.058 g, 0.30 mmol) in TFA (0.1 mL) and HFIP (2 mL) was stirred at 25° C. for 12 h. The reaction mixture was concentrated under reduced pressure to provide the title compound (0.025 g) as a yellow oil, which was used directly in the next step without further purification.

### 1-(6-((S)-3-((S)-2-Cyano-2-methylazetidin-1-yl)-1,1,1-trifluoro-2-hydroxy-3-oxopropan-2-yl)benzo[d]thiazol-2-yl)-3-(4-fluorophenyl)urea (333) and 1-(6-((S)-3-((R)-2-cyano-2-methylazetidin-1-yl)-1,1,1-trifluoro-2-hydroxy-3-oxopropan-2-yl)benzo[d]thiazol-2-yl)-3-(4-fluorophenyl)urea (344)

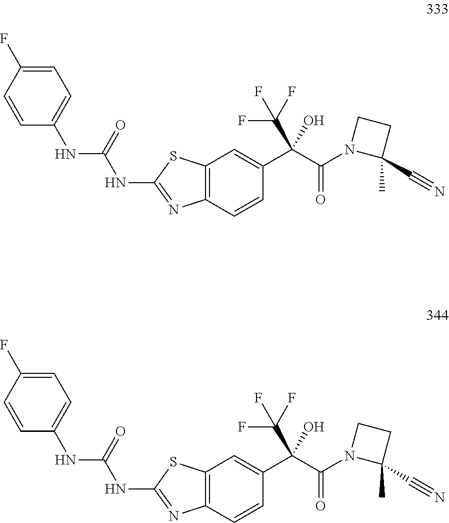 [SMILES: C[C@@]1(C#N)CCN1C(=O)[C@@](O)(c1ccc2nc(NC(=O)Nc3ccc(F)cc3)sc2c1)C(F)(F)F ; C[C@]1(C#N)CCN1C(=O)[C@@](O)(c1ccc2nc(NC(=O)Nc3ccc(F)cc3)sc2c1)C(F)(F)F]

To a solution of (2S)-3,3,3-trifluoro-2-[2-[(4-fluorophenyl) carbamoylamino]-1,3-benzothiazol-6-yl]-2-hydroxy-propanoic acid (0.06 g, 0.14 mmol) and 2-methylazetidine-2-carbonitrile (16.1 mg, crude) in DMF (2 mL) was added HATU (106 mg, 0.28 mmol) and DIPEA (54.2 mg, 0.42 mmol). After stirring at 25° C. for 2 h, the reaction was diluted with H₂O (5 mL) and extracted with EtOAc (5 mL×3). The combined organic layer was washed with brine, dried over Na₂SO₄, filtered and concentrated under reduced pressure. The residue was purified by reverse phase HPLC (H₂O (0.2% FA)-ACN], gradient 25-55% elution) to provide 10 mg of the diastereomer mixture of 333 and 344, which was further purified by reverse phase HPLC (H₂O) (0.2% FA)-ACN], gradient 30-70% elution) to provide the title compounds 333 (1.8 mg, 18%) as a white solid and 344 (1.5 mg, 25%) as a white solid. Data for 333: MS (ESI): mass calcd, for C₂₂H₁₇F₄N₅O₃S: 507.10, found: 508.1 [M+H]⁺. ¹H NMR (400 MHz, DMSO-d6) δ ppm 9.26 (s, 1H), 8.06 (s, 1H), 8.02 (s, 1H), 7.71 (d, J=8.8 Hz, 1H), 6.62-6.65 (m, 1H), 7.48-7.55 (m, 3H), 7.17 (t, J=8.8 Hz, 2H), 4.22-4.29 (m, 1H), 3.37-3.42 (m, 1H), 2.60-2.64 (m, 1H), 2.14-2.17 (m, 1H), 1.68 (s, 3H). Data for 344: MS (ESI): mass calcd, for C₂₂H₁₇F₄N₅O₃S: 507.10, found: 508.1 [M+H]⁺. ¹H NMR (400 MHz, DMSO-d6) δ ppm 9.26 (s, 1H), 8.06 (s, 1H), 8.02 (s, 1H), 7.72 (s, 1H), 7.55-7.56 (m, 1H), 7.47-7.54 (m, 3H), 7.17 (t, J=8.8 Hz, 2H), 4.16-4.23 (m, 1H), 3.41-3.42 (m, 1H), 2.62-2.68 (m, 1H), 2.26-2.32 (m, 1H), 1.77 (s, 3H). Stereochemistry was arbitrarily assigned.

### Example 337. 1-(6-((S)-3-((S)-2-(1H-Tetrazol-S-yl)azetidin-1-yl)-1,1,1-trifluoro-2-hydroxy-3-oxopropan-2-yl)benzo[d]thiazol-2-yl)-3-(4-fluorophenyl)urea (337)

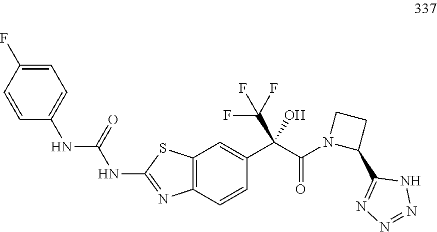 [SMILES: O=C(Nc1ccc(F)cc1)Nc1nc2ccc([C@](O)(C(=O)N3CC[C@H]3c3nnn[nH]3)C(F)(F)F)cc2s1]

### Synthetic Scheme:

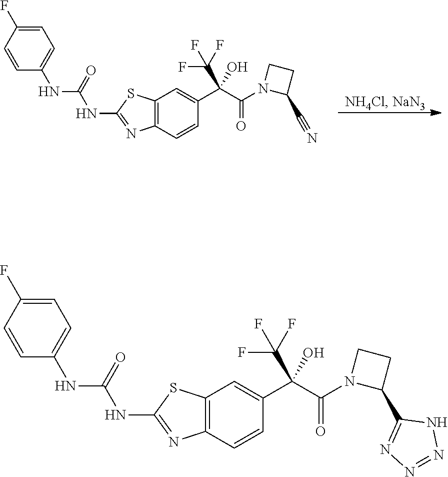

### 1-(6-((S)-3-((S)-2-(1H-Tetrazol-5-yl)azetidin-1-yl)-1,1,1-trifluoro-2-hydroxy-3-oxopropan-2-yl)benzo[d]thiazol-2-yl)-3-(4-fluorophenyl)urea (337)

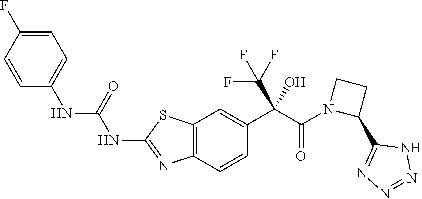 [SMILES: O=C(Nc1ccc(F)cc1)Nc1nc2ccc([C@](O)(C(=O)N3CC[C@H]3c3nnn[nH]3)C(F)(F)F)cc2s1]

A mixture of 1-(6-((S)-3-((S)-2-cyanoazetidin-1-yl)-1,1,1-trifluoro-2-hydroxy-3-oxopropan-2-yl)benzo[d]thiazol-2-yl)-3-(4-fluorophenyl)urea (Example 327) (0.03 g, 0.061 mmol), NH₄Cl (3.6 mg, 0.067 mmol) and NaN₃ (4.4 mg, 0.067 mmol) in DMF (1 mL) was degassed and purged with N₂ 3×. It was then stirred at 90° C. under N₂ for 16 h. The reaction was poured into H₂O (5 mL) and adjusted to pH=9 with aqueous NaHCO₃. The resulting solution was diluted with H₂O (5 mL) and extracted with EtOAc (5 mL×3). The combined organic layer was dried over Na₂SO₄, filtered, and concentrated. The crude was purified by reverse phase HPLC (gradient elution, 25-55% ACN in H₂O, with 0.2% FA as a modifier) to afford the title compound (337) (1.2 mg, 3.7%) as a white solid. MS (ESI): mass calcd. for C₂₁H₁₆F₄N₈O₃S: 536.10, found: 536.7 [M+H]+. ¹H NMR (400 MHz, DMSO-d6) δ ppm 10.99 (s, 1H), 9.25 (s, 1H), 8.14 (s, 1H), 7.50-7.72 (m, 5H), 7.18 (t, J=8.8 Hz, 2H), 5.47-6.06 (m, 1H), 3.93-4.42 (m, 2H), 2.66-2.73 (m, 1H), 2.43-2.45 (m, 1H), 2.20-2.22 (m, 1H).

Examples 338 was synthesized in similar procedures as described in Example 337.

### Example 342. 1-(4-Fluorophenyl)-3-(6-((S)-1,1,1-trifluoro-2-hydroxy-3-((S)-2-(1-hydroxycyclopropyl)azetidin-1-yl)-3-oxopropan-2-yl)benzo[d]thiazol-2-yl)urea (342)

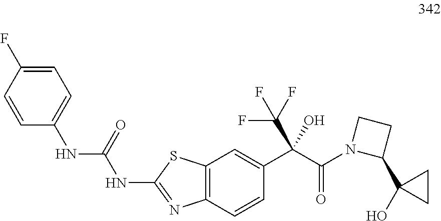 [SMILES: O=C(Nc1ccc(F)cc1)Nc1nc2ccc([C@](O)(C(=O)N3CC[C@H]3C3(O)CC3)C(F)(F)F)cc2s1]

### Synthetic Scheme:

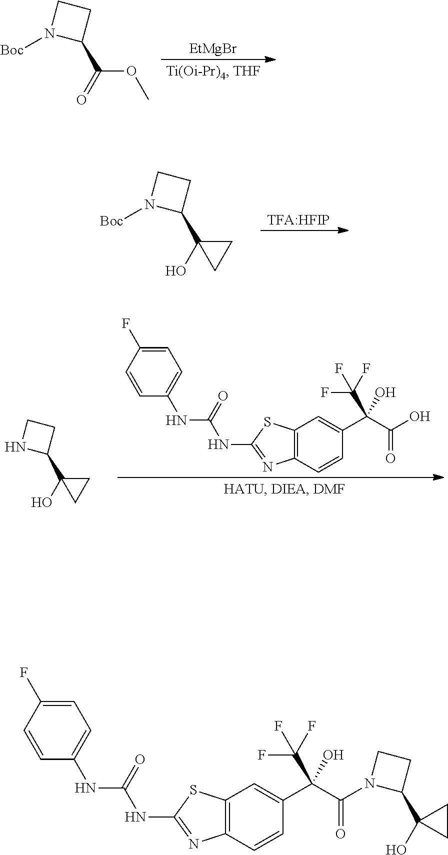

### tert-Butyl(2S)-2-(1-hydroxycyclopropyl)azetidine-1-carboxylate

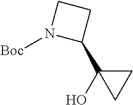 [SMILES: CC(C)(C)OC(=O)N1CC[C@H]1C1(O)CC1]

To a solution of 1-(tert-butyl) 2-methyl (S)-azetidine-1,2-dicarboxylate (1 g, 4.65 mmol) in THF (10 mL) was added EtMgBr (3 M, 3.9 mL) and Ti(Oi-Pr)₄ (660 mg, 2.32 mmol) at 0° C. After stirring at 25° C. for 12 b, the reaction was washed with saturated NH₄Cl (10 mL) and extracted with EtOAc (10 mL×3). The combined organic layer was washed with brine, dried over Na₂SO₄, filtered and concentrated under reduced pressure. The residue was purified by prep-TLC (SiO₂, Petroleum ether:Ethyl acetate=3:1) to provide the title compound (63 mg, 6.4%) as a brown oil. ¹H NMR (400 MHz, CDCl₃) δ ppm 3.20-3.47 (m, 2H), 3.01-3.19 (m, 2H), 2.88-3.00 (m, 1H), 2.19-2.30 (m, 1H), 1.67-1.80 (m, 2H), 1.45 (s, 9H), 0.71-1.13 (m, 2H).

### 1-[(2S)-Azetidin-2-yl]cyclopropanol

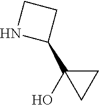

A solution of tert-butyl (2S)-2-(1-hydroxycyclopropyl)azetidine-1-carboxylate (63 mg, 0.3 mmol) in TFA (0.1 mL) and HFIP (2 mL) was stirred at 25° C. for 12 h. The reaction was concentrated under reduced pressure and lyophilized to provide the crude title compound (63 mg. TFA salt) as a brown oil, which was used in the next step without further purification.

### 1-(4-Fluorophenyl)-3-(6-((S)-1,1,1-trifluoro-2-hydroxy-3-((S)-2-(1-hydroxycyclopropyl)azetidin-1-yl)-3-oxopropan-2-yl)benzo[d]thiazol-2-yl)urea (342)

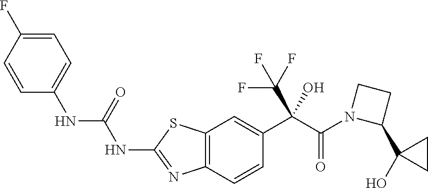 [SMILES: O=C(Nc1ccc(F)cc1)Nc1nc2ccc([C@](O)(C(=O)N3CC[C@H]3C3(O)CC3)C(F)(F)F)cc2s1]

To a solution of (2S)-3,3,3-trifluoro-2-[2-[(4-fluorophenyl) carbamoylamino]-1,3-benzothiazol-6-yl]-2-hydroxy-propanoic acid (100 mg, 0.23 mmol) in DMF (3 mL) was added DIPEA (90.3 mg, 0.7 mmol) and HATU (177 mg, 0.47 mmol) at 0° C. After 15 min. 1-[(2S)-azetidin-2-yl]cyclopropanol (63 mg, 0.28 mmol, TFA salt) was added and the reaction was stirred at 25° C. for 1 h. The reaction mixture was diluted with H₂O (9 mL) and extracted with EtOAc (5 mL×2). The combined organic layer was washed with brine, dried over Na₂SO₄, filtered and concentrated under reduced pressure. The residue was purified by reverse phase HPLC (H₂O) (0.2% FA)-ACN, gradient 30-60% elution) to provide the title compound (342) (9.4 mg, 7.7%) as a white solid. MS (ESI): mass calcd, for C₂₃H₂₀F₄N₄O₄S: 524.11, found: 525.1 [M+H]⁺, ¹H NMR (400 MHz, DMSO-d₆) δ ppm 11.15 (s, 1H), 9.32 (s, 1H), 8.25 (t, J=5.6 Hz, 1H), 8.15 (s, 1H), 7.79 (s, 1H), 7.66 (s, 2H), 7.51-7.56 (m, 2H), 7.17 (t, J=8.8 Hz, 2H), 3.11-3.18 (m, 3H), 2.95-3.02 (m, 1H), 2.73-2.82 (m. 1H), 2.01-2.09 (m, 1H), 1.70-1.75 (m, 1H), 1.51-1.57 (m, 2H).

### Example 348. (1R,2S)-2-((S)-3,3,3-Trifluoro-2-(2-(3-(4-fluorophenyl)ureido)benzo[d]thiazol-6-yl)-2-hydroxypropanamido)cyclobutyl methylcarbamate (348)

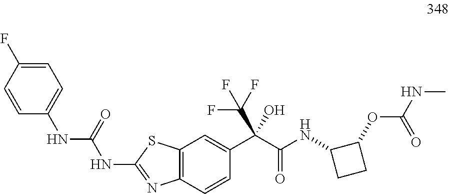

### Synthetic Scheme:

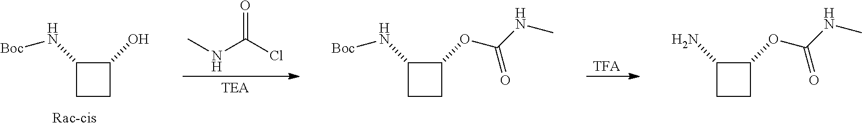

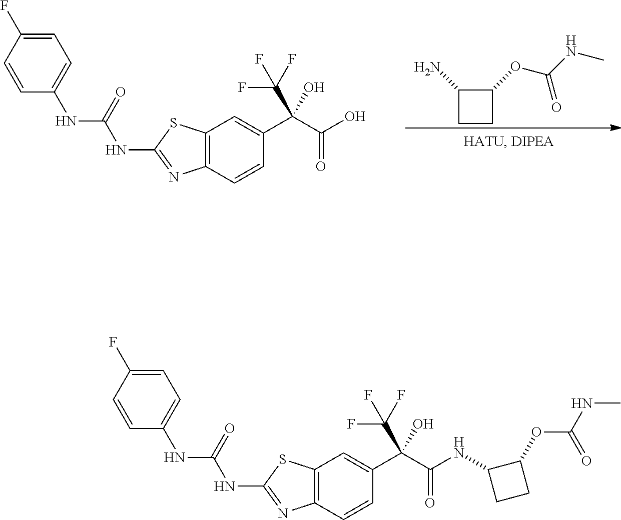

### Rac-tert-butyl ((1S,2R)-2-((methylcarbamoyl)oxy)cyclobutyl)carbamate

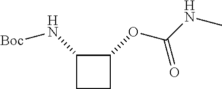 [SMILES: CNC(=O)O[C@@H]1CC[C@@H]1NC(=O)OC(C)(C)C]

To an ice cold solution of rac-ter-butyl ((1S,2R)-2-((methylcarbamoyl)oxy)cyclobutyl)carbamate (0.1 g, 0.53 mmol) in DCM (5 mL) was added TEA (372 μL, 2.67 mmol), pyridine (86 μL. 1.07 mmol) and N-methylcarbamoyl chloride (59.9 mg, 0.64 mmol). The resulting solution was allowed to stir at 20° C. for 12 h. The reaction was diluted with sat. NaHCO₃ solution (15 mL) and extracted with DCM (15 mL×3). The combined organic layer was washed with brine, dried over Na₂SO₄, filtered and concentrated. The crude was purified by prep-TLC (33% EtOAc in PE) to give the title compound (0.01 g, 7.7%) as a yellow solid. ¹H NMR (400 MHz, CD₃OD) δ ppm 5.15 (s, 1H), 5.01 (s, 1H), 4.70-4.72 (m, 1H), 4.31 (t, J=6.4 Hz, 1H), 2.82 (d, J=3.2 Hz, 3H), 2.20 (br s, 2H), 1.97 (br s, 2H), 1.45 (s, 9H).

### Rac-(1R,2S)-2-aminocyclobutyl methylcarbamate

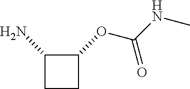

Rac-(1R,2S)-2-aminocyclobutyl methylcarbamate (0.05 g, 0.20 mmol) was dissolved in a solution of DCM (1 mL) and TFA (0.2 mL). After stirring at 25° C. for 2 h, the reaction was concentrated under reduced pressure to afford the title compound (0.043 g, 81.4%) as a yellow oil, which was directly used in the next step without further purification. ¹H NMR (400 MHz, CDCl₃) δ ppm 5.15 (s, 1H), 4.32 (t, J=6.8 Hz, 1H), 4.02 (s, 1H), 2.73-2.89 (m, 3H), 2.18-2.52 (m, 4H), 1.34-1.50 (m, 2H).

### Rac-(1R,2S)-2-((S)-3,3,3-trifluoro-2-(2-(3-(4-fluorophenyl)ureido)benzo[d]thiazol-6-yl)-2-hydroxypropanamido)cyclobutyl methylcarbamate

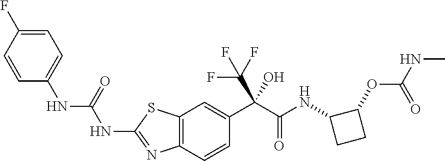

To a solution of (2S)-3,3,3-trifluoro-2-[2-[(4-fluorophenyl) carbamoylamino]-1,3-benzothiazol-6-yl]-2-hydroxy-propanoic acid (described in Example 38, 99.3 mg, 0.23 mmol) in DMF (1 mL) were added DIPEA (201.4 μL, 1.16 mmol), HATU (175.8 mg, 0.46 mmol) and rac-(1R,2S)-2-aminocyclobutyl methylcarbamate (0.04 g, 0.28 mmol). The resulting solution was stirred at 25° C. for 2 h. After completion, it was diluted with H₂O (10 mL) and extracted with EtOAc (10 mL×3). The combined organic layer was washed with brine, dried over Na₂SO₄, filtered, and concentrated. The crude was purified by reverse phase HPLC (gradient separation, 20-55% ACN in H₂O with 0.2% FA as a modifier) to afford the title compound (348) (6 mg, 30%, diastereomeric mixture) as a white solid, MS (ESI): mass calcd. for C₂₃H₂₁F₄N₅O₅S: 555.12, found: 556.1 [M+H]⁺. ¹H NMR (400 MHz, DMSO-d₆) δ ppm 10.85-11.46 (m, 1H), 9.33 (s, 1H), 8.08-8.19 (m, 1H), 7.92-8.01 (m, 1H), 7.83 (t, J=7.6 Hz, 1H), 7.64 (d, J=17.2 Hz, 2H), 7.51-7.58 (m, 2H), 7.17 (t, J=8.8 Hz, 2H), 6.78-7.02 (m, 1H), 4.91-5.08 (m, 1H), 4.34-4.51 (m, 1H), 2.51-2.62 (m, 2H), 2.39 (d, J=4.8 Hz, 1H), 1.86-2.18 (m, 4H) as a 2:1 diastereomer mixture.

### Example 353. (S)—N-Cyclopropyl-2-(cyclopropylamino)-3,3,3-trifluoro-2-(2-(3-(4-fluorophenyl)ureido)benzo[d]thiazol-6-yl)propanamide (353)

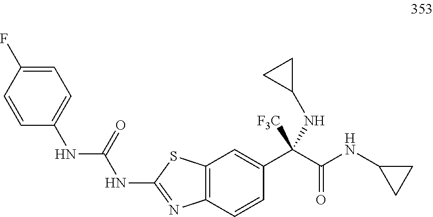 [SMILES: O=C(Nc1ccc(F)cc1)Nc1nc2ccc([C@](NC3CC3)(C(=O)NC3CC3)C(F)(F)F)cc2s1]

### Synthetic Scheme:

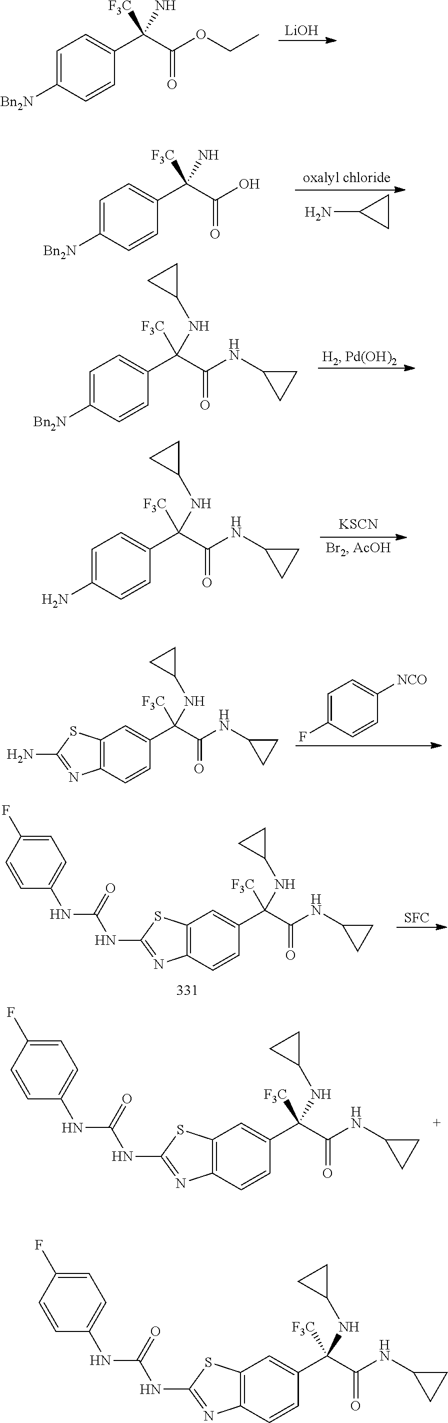

### (S)-2-(4-(Dibenzylamino)phenyl)-3,3,3-trifluoro-2-hydroxypropanoic Acid

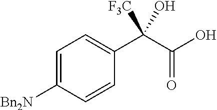

A suspension of ethyl (S)-2-(4-(dibenzylamino)phenyl)-3,3,3-trifluoro-2-hydroxypropanoate (550 mg, 1.24 mmol) and LiOH·H₂O (149 mg, 6.2 mmol) in THF (4 mL), MeOH (1 mL), and H₂O (1 mL) was stirred at rt for 16 h. After completion, the reaction solvent was removed. The resulting residue was purified by reverse phase HPLC (gradient elution, 25-65% ACN in H₂O with 0.1% TFA as a modifier) to provide the title product (773 mg, with impurities) as a white solid. MS (ESI): mass calcd, for C₂₃H₂₀F₃NO₃: 415.14, found: 415.90 [M+H]⁺.

### N-Cyclopropyl-2-(cyclopropylamino)-2-(4-(dibenzylamino)phenyl)-3,3,3-trifluoropropanamide

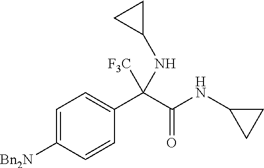

To an ice cold solution of (S)-2-(4-(dibenzylamino)phenyl)-3,3,3-trifluoro-2-hydroxypropanoic acid (773 mg, impure) in DCM (18.6 mL) was added oxalyl chloride (0.32 mL, 3.72 mmol) and a drop of DMF. The reaction was stirred at 0° C. and slowly warmed to rt. After 2 h, cyclopropylamine (1.3 mL, 18.6 mmol) was added. After stirring at rt overnight, the reaction was quenched with H₂O (20 mL) and extracted with EtOAc (50 mL×3). The combined organic layer was washed with brine, dried over Na₂SO₄, filtered, and concentrated under reduced pressure. The resulting residue was purified by silica gel column chromatography (gradient elution, 0-20% MeOH in DCM) to afford the title compound (390 mg) as a white solid. MS (ESI): mass calcd, for C₂₉H₃₀F₃NO: 493.23, found: 494.00 [M+H]⁺. Racemization occurred at this step.

### 2-(4-Aminophenyl)-N-cyclopropyl-2-(cyclopropylamino)-3,3,3-trifluoropropanamide

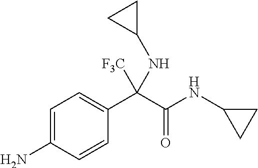 [SMILES: Nc1ccc(C(NC2CC2)(C(=O)NC2CC2)C(F)(F)F)cc1]

Pd(OH)₂ (111 mg, 0.16 mmol) was added to a solution of N-cyclopropyl-2-(cyclopropylamino)-2-(4-(dibenzylamino)phenyl)-3,3,3-trifluoropropanamide (390 mg, 0.79 mmol) in EtOH (7.9 mL). The resulting suspension was purged with H₂ for 5 minutes and further stirred under a H₂ balloon overnight. After completion, the reaction was filtered through a syringe filter. The filtrate was concentrated to afford the crude title compound (244 mg, 99%) as a colorless oil, which was directly used in the next step without further purification. MS (ESI): mass calcd, for C₁₅H₁₈F₃N₃O: 313.14, found: 313.90 [M+H]⁺.

### 2-(2-Aminobenzo[d]thiazol-6-yl)-N-cyclopropyl-2-(cyclopropylamino)-3,3,3-trifluoropropanamide

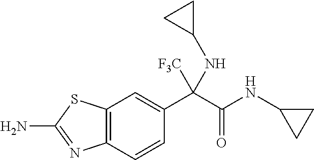 [SMILES: Nc1nc2ccc(C(NC3CC3)(C(=O)NC3CC3)C(F)(F)F)cc2s1]

To a solution of 2-(4-aminophenyl)-N-cyclopropyl-2-(cyclopropylamino)-3,3,3-trifluoropropanamide (244 mg, 0.78 mmol) in AcOH (10 mL) was added KSCN (265 mg, 2.73 mmol) at 25° C. After stirring at this temperature for 1 h, a solution of Br₂ (44 μg, 0.86 mmol) in AcOH (1.4 mL) was added. After 3 h, the reaction suspension was filtered through a filter paper. The filtrate was concentrated and then dissolved in EtOAc (30 mL). The organic layer was washed with sat. NaHCO₃ (10 mL). The aqueous layer was extracted with EtOAc (50 mL×2). The combined organic layer was washed with brine, dried over anhydrous Na₂SO₄, filtered, concentrated, and purified by silica gel column chromatography (gradient elution, 40-80% EtOAc in hexanes) to afford the title compound (215 mg, 75%) as a yellow oil. MS (ESI): mass calcd, for C₁₆H₁₇F₃N₄OS: 370.11, found: 370.85 [M+H]⁺.

### N-Cyclopropyl-2-(cyclopropylamino)-3,3,3-trifluoro-2-(2-(3-(4-fluorophenyl)ureido)benzo[d]thiazol-6-yl)propenamide (331)

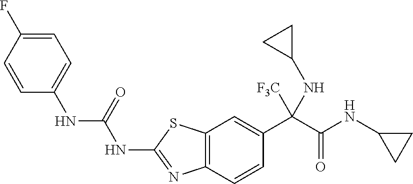 [SMILES: O=C(Nc1ccc(F)cc1)Nc1nc2ccc(C(NC3CC3)(C(=O)NC3CC3)C(F)(F)F)cc2s1]

To a solution of 2-(2-aminobenzo[d]thiazol-6-yl)-N-cyclopropyl-2-(cyclopropylamino)-3,3,3-trifluoropropanamide (30 mg, 0.081 mmol) in THE (0.81 mL) was added 4-fluorophenyl isocyanate (14.9 μL, 0.12 mmol) at rt. After 3 h, the reaction was quenched with H₂O (3 mL) and extracted with EtOAc (10 mL×3). The combined organic layer was washed with brine (10 mL×3), dried over anhydrous Na₂SO₄, filtered, and concentrated. The crude was purified by reverse phase HPLC (gradient separation, 30-80% ACN in H₂O with 0.1% TFA as a modifier) to obtain the title product (331) (32.7 mg, 80%, racemic) as a white solid. MS (ESI): mass calcd, for C₂₃H₂₁F₄N₅O₂S: 507.14 found: 507.90 [M+H]⁺. ¹H-NMR (400 MHz; DMSO-d6) δ ppm 10.86 (br s, 1H), 9.21 (d, J=1.1 Hz, 1H), 8.02 (d, J=3.7 Hz, 2H), 7.63 (dd, J=7.7, 0.9 Hz, 1H), 7.53 (dd, J=8.7, 4.9 Hz, 2H), 7.39 (dd, J=8.5, 1.3 Hz, 1H), 7.17 (t, J=8.9 Hz, 2H), 2.75 (qd, J=7.3, 3.6 Hz, 1H), 2.05-2.10 (m, 1H), 0.64-0.69 (m, 2H), 0.36-0.52 (m, 6H).

### Example 352 and 353, (R)—N-Cyclopropyl-2-(cyclopropylamino)-3,3,3-trifluoro-2-(2-(3-(4-fluorophenyl)ureido)benzo[d]thiazol-6-yl)propanamide (352) and (S)—N-Cyclopropyl-2-(cyclopropylamino)-3,3,3-trifluoro-2-(2-(3-(4-fluorophenyl)ureido)benzo[d]thiazol-6-yl)propanamide (353)

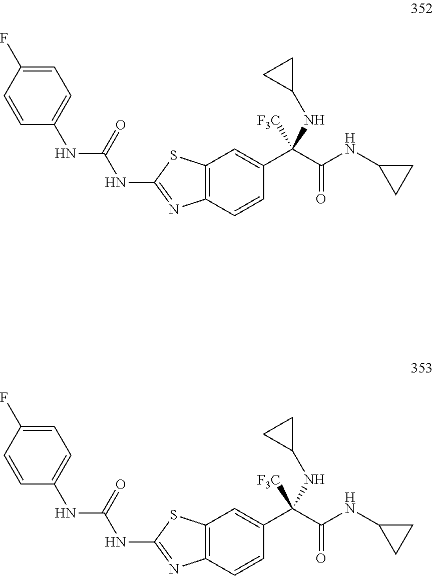 [SMILES: O=C(Nc1ccc(F)cc1)Nc1nc2ccc([C@@](NC3CC3)(C(=O)NC3CC3)C(F)(F)F)cc2s1 ; O=C(Nc1ccc(F)cc1)Nc1nc2ccc([C@](NC3CC3)(C(=O)NC3CC3)C(F)(F)F)cc2s1]

Example 331 was separated by chiral SFC (column: DAICEL CHIRALPAK AD (250 mm*30 mm, 10 μm); mobile phase: 40% EtOH (0.1% NH₃H₂O), isocratic elution) to provide the title compound (352). MS (ESI): mass calcd, for C₂₃H₂₁F₄N₅O₂S: 507.14, found: 508.1 [M+H]⁺. ¹H NMR (400 MHz, DMSO-d6) δ ppm 9.13-9.26 (m, 1H), 8.03 (d, J=4.0 Hz, 1H), 8.00 (s, 1H), 7.49-7.66 (m, 3H), 7.37 (d, J=9.2 Hz, 1H), 7.16 (t, J=8.8 Hz, 2H), 3.90 (s, 1H), 2.72-2.79 (m, 1H), 2.08 (m, 1H), 0.63-0.69 (m, 2H), 0.37-0.51 (m, 6H). And to provide the title compound (353) as a white solid. MS (ESI): mass calcd, for C₂₃H₂₁F₄N₅O₂S: 507.14, found: 508.1 [M+H]⁺. ¹H NMR (400 MHz, DMSO-d6) δ ppm 9.21 (s, 1H), 8.03 (d, J=3.6 Hz, 1H), 8.00 (s, 1H), 7.49-7.65 (m, 3H), 7.37 (d, J=8.4 Hz, 1H), 7.16 (t, J=8.8 Hz, 2H), 3.90 (s, 1H), 2.70-2.79 (m, 1H), 2.03-2.13 (m, 1H), 0.62-0.71 (m, 2H), 0.37-0.52 (m, 6H). Stereochemistry was arbitrarily assigned.

Examples 336, 350, and 351 were synthesized in similar procedures as described in Example 353.

### Example 347. 1-(4-Fluorophenyl)-3-(6-((S)-1,1,1-trifluoro-2-hydroxy-3-((S)-2-((S)-1-hydroxyethyl)azetidin-1-yl)-3-oxopropan-2-yl)benzo[d]thiazol-2-yl)urea (347)

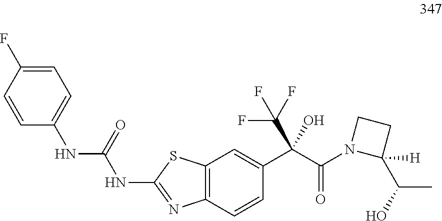 [SMILES: C[C@H](O)[C@@H]1CCN1C(=O)[C@@](O)(c1ccc2nc(NC(=O)Nc3ccc(F)cc3)sc2c1)C(F)(F)F]

### Synthetic Scheme:

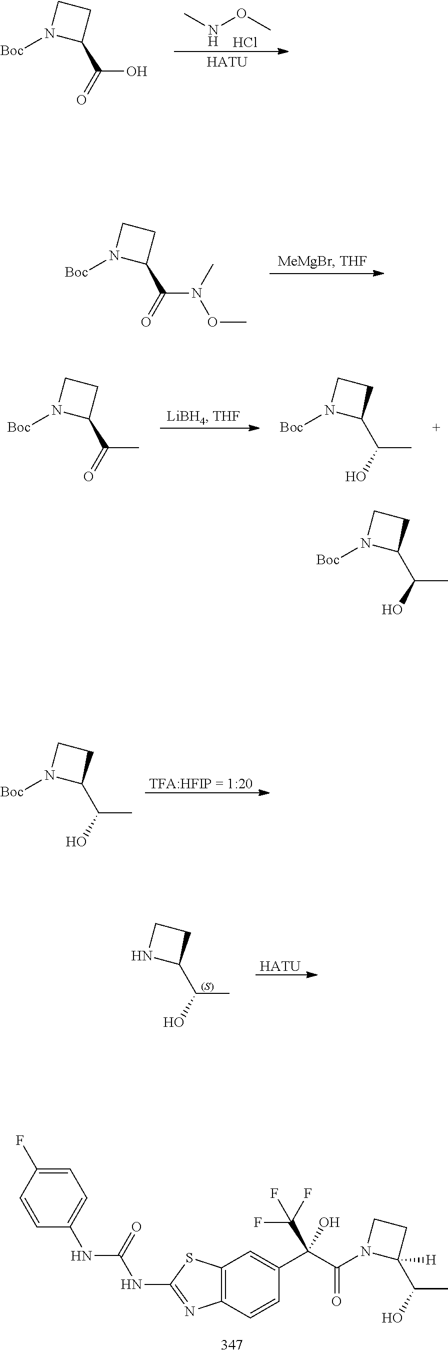

### tert-Butyl(2S)-2-[methoxy(methyl)carbamoyl]azetidine-1-carboxylate

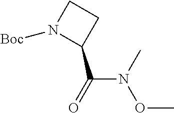 [SMILES: CON(C)C(=O)[C@@H]1CCN1C(=O)OC(C)(C)C]

To a solution of (2S)-1-tert-butoxycarbonylazetidine-2-carboxylic acid (1 g, 4.97 mmol) in DMF (10 mL) was added DIPEA (1.93 g, 14.9 mmol) and HATU (3.78 g, 9.94 mmol). After stirring at 25° C. for 15 min, N-methoxymethanamine (581.7 mg, 5.96 mmol) was added and the reaction was stirred at 25° C. for 15 h. The reaction was diluted with H₂O (45 mL) and extracted with EtOAc (25 mL×3). The combined organic layer was washed with brine, dried over Na₂SO₄, filtered and concentrated under reduced pressure. The residue was purified by silica gel column chromatography (Petroleum ether:Ethyl acetate=5:1 to 0:1) to provide the title compound (0.96 g, 79%) as a yellow oil. ¹H NMR (400 MHz, DMSO-d6) δ ppm 4.90-5.01 (m, 1H), 3.79-3.85 (m, 1H), 3.74 (s, 1H), 3.65 (s, 3H), 3.12 (s, 3H), 2.41-2.49 (m, 1H), 1.90-1.99 (m, 1H), 1.34 (s, 9H).

### tert-Butyl (2S)-2-acetylazetidine-1-carboxylate

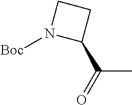

To a solution of tert-butyl(2S)-2-[methoxy(methyl)carbamoyl]azetidine-1-carboxylate (0.3 g, 1.23 mmol) in THF (3 mL) was added MeMgBr (1 M, 2.46 mL). After stirring at −78° C. for 2 h, the reaction was quenched with saturated NH₄Cl (5 mL) and extracted with EtOAc (5 mL×3). The combined organic layer was washed with brine, dried over Na₂SO₄, filtered and concentrated under reduced pressure. The residue was purified by silica gel column chromatography (Petroleum ether:Ethyl acetate=1:0 to 0:1) to provide the title compound (0.13 g, 53%) as a white solid. ¹H NMR (400 MHz, DMSO-d₆) δ ppm 4.55-4.62 (m, 1H), 3.71-3.83 (m, 2H), 2.36-2.46 (m, 1H), 2.15 (s, 3H), 1.98-2.07 (m, 1H), 1.32-1.41 (m, 9H).

### tert-Butyl (S)-2-((S)-1-hydroxyethyl)azetidine-1-carboxylate and tert-Butyl (S)-2-((R)-1-hydroxyethyl)azetidine-1-carboxylate

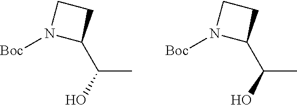

To a solution of tert-butyl(2S)-2-acetylazetidine-1-carboxylate (0.58 g, 2.91 mmol) in THF (6 mL) was added LiBH₄ (2 M, 2.18 mL) at 0° C. After stirring at 25° C. for 1 h, the reaction was quenched with aqueous NH₄Cl solution (5 mL) and extracted with EtOAc (10 mL×3). The combined organic layer was washed with brine, dried over Na₂SO₄, filtered and concentrated under reduced pressure. The residue was purified by silica gel column chromatography (Petroleum ether:Ethyl acetate=100:1 to 0:1) to provide the title compounds (S,S) (0.2 g, 34%) and (S,R) (0.1 g, 17%) as yellow oil. Stereochemistry was arbitrarily assigned.

(S,S): ¹H NMR (400 MHz, DMSO-d₆) δ ppm 4.05-4.12 (m, 1H), 3.81-3.94 (m, 2H), 3.72-3.78 (m, 1H), 2.12-2.21 (m, 1H), 1.82-1.92 (m, 1H), 1.46 (s, 9H), 1.06 (d, J=6.4 Hz, 3H).
(S,R): ¹H NMR (400 MHz, DMSO-d₆) δ ppm 4.29-4.39 (m, 1H), 3.93-4.00 (m, 1H), 3.81-3.90 (m, 1H), 3.69-3.76 (m, 1H), 2.05-2.14 (m, 2H), 1.45 (s, 9H), 1.15 (d, J=6.4 Hz, 3H).

### (S)-1-((S)-Azetidin-2-yl) ethan-1-ol

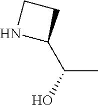

A solution of tert-Butyl (S)-2-((S)-1-hydroxyethyl)azetidine-1-carboxylate (0.04 g, 0.2 mmol) in TFA (0.1 mL) and HFIP (2 mL) was stirred at 25° C. for 12 h. The reaction was concentrated under reduced pressure to provide the title compound (0.036 g, TFA salt) as a white oil, which was used in the next step without further purification. 1-(4-Fluorophenyl)-3-(6-((S)-1,1,1-trifluoro-2-hydroxy-3-((S)-2-((S)-1-hydroxyethyl)azetidin-1-yl)-3-oxopropan-2-yl)benzo[d]thiazol-2-yl)urea (347)

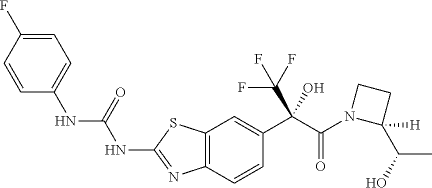 [SMILES: C[C@H](O)[C@@H]1CCN1C(=O)[C@@](O)(c1ccc2nc(NC(=O)Nc3ccc(F)cc3)sc2c1)C(F)(F)F]

To a solution of (2S)-3,3,3-trifluoro-2-[2-[(4-fluorophenyl) carbamoylamino]-1,3-benzothiazol-6-yl]-2-hydroxy-propanoic acid (0.06 g, 0.14 mmol) in DMF (2 mL) was added DIPEA (90.3 mg, 0.7 mmol) and HATU (106.3 mg, 0.28 mmol). After heating at 40° C. for 15 min, (S)-1-((S)-azetidin-2-yl) ethan-1-ol (36 mg, TFA salt) was added and the reaction was stirred at 40° C. for 2 h. The reaction mixture was diluted with H₂O (10 mL) and extracted with EtOAc (10 mL×3). The combined organic layer was washed with brine, dried over Na₂SO₄, filtered and concentrated under reduced pressure. The residue was purified by reverse phase HPLC (H₂O (0.2% FA)-ACN, gradient 25-60% elution) to provide the title compound (347) (0.05 g, 70%) as a white solid. MS (ESI): mass calcd, for C₂₂H₂₀F₄N₄O₄S: 512.11, found: 513.1 [M+H]⁺. ¹H NMR (400 MHz, DMSO-d6) δ ppm 11.15 (s, 1H), 9.39 (s, 1H), 8.07 (s, 1H), 7.74 (s, 1H), 7.67 (d, J=8.4 Hz, 1H), 7.50-7.59 (m, 4H), 7.16 (t, J=8.8 Hz, 2H), 4.87 (d, J=3.6 Hz, 1H), 4.23-4.27 (m, 1H), 4.06-4.10 (m, 1H), 3.93 (d, J=4.0 Hz, 1H), 1.88-1.99 (m, 2H), 1.07 (d, J=6.4 Hz, 3H). Stereochemistry was arbitrarily assigned.

Example 369 was synthesized in similar procedures as described in Example 347, using tert-Butyl (S)-2-((R)-1-hydroxyethyl)azetidine-1-carboxylate.

### Example 366. Methyl (S)-3-(3,3,3-trifluoro-2-(2-(3-(4-Fluorophenyl)ureido)benzo[d]thiazol-6-yl)-2-hydroxypropanamido)azetidine-1-carboxylate (366)

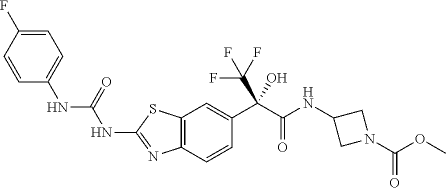 [SMILES: COC(=O)N1CC(NC(=O)[C@@](O)(c2ccc3nc(NC(=O)Nc4ccc(F)cc4)sc3c2)C(F)(F)F)C1]

### Synthetic Scheme:

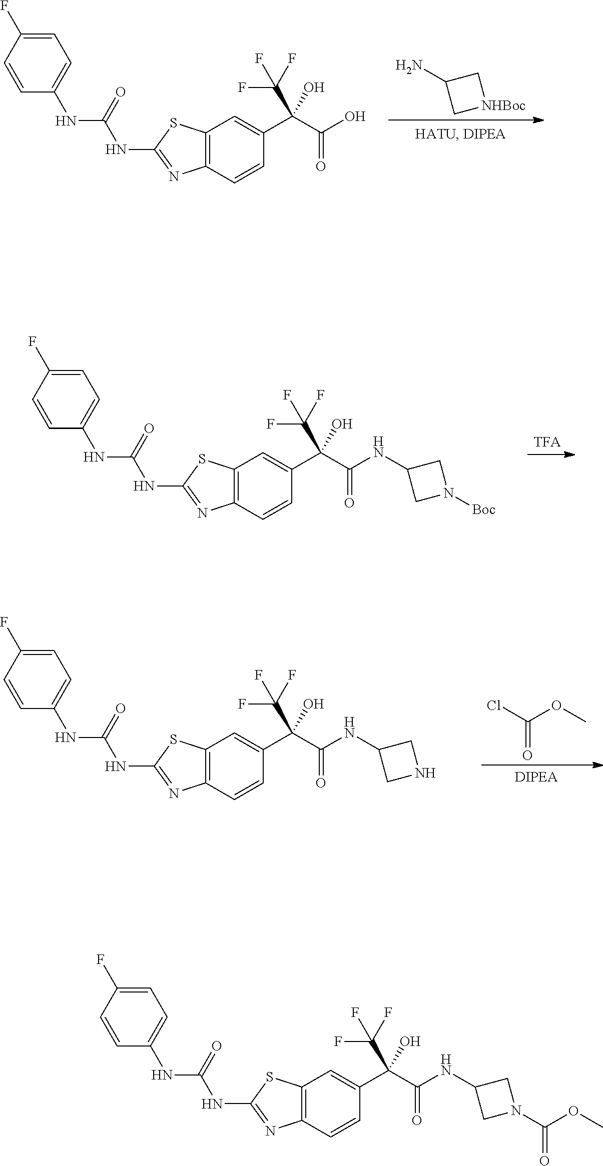

### tert-Butyl (S)-3-(3,3,3-trifluoro-2-(2-(3-(4-fluorophenyl)ureido)benzo[d]thiazol-6-yl)-2-Hydroxypropanamido)azetidine-1-carboxylate

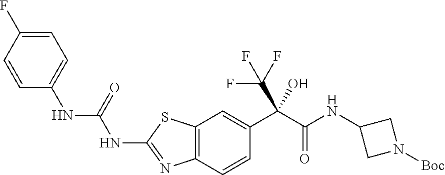 [SMILES: CC(C)(C)OC(=O)N1CC(NC(=O)[C@@](O)(c2ccc3nc(NC(=O)Nc4ccc(F)cc4)sc3c2)C(F)(F)F)C1]

To a solution of (2S)-3,3,3-trifluoro-2-[2-[(4-fluorophenyl) carbamoylamino]-1,3-benzothiazol-6-yl]-2-hydroxy-propanoic acid (described in Example 38, 0.2 g, 0.47 mmol) in DMF (2 mL) was added DIPEA (0.24 mL, 1.4 mmol), HATU (354.2 mg, 0.93 mmol) and tert-butyl 3-aminoazetidine-1-carboxylate (96.3 mg, 0.56 mmol). It was then stirred at 25° C. for 12 h under N₂. After completion, the reaction was diluted with H₂O (5 mL) and extracted with EtOAc (5 mL×3). The combined organic layer was washed with brine, dried over Na₂SO₄, filtered, and concentrated. The crude was purified by prep-TLC (50% EtOAc in PE) to give the title compound (0.08 g, 29.4%) as a white solid. MS (ESI): mass calcd, for C₂₅H₂₅F₄N₅O₅S: 583.15 found: 528.2 [M−56+1]⁺.

### (2S)—N-(Azetidin-3-yl)-3,3,3-trifluoro-2-[2-[(4-fluorophenyl) carbamoylamino]-1,3-benzothiazol-6-yl]-2-hydroxy-propanamide

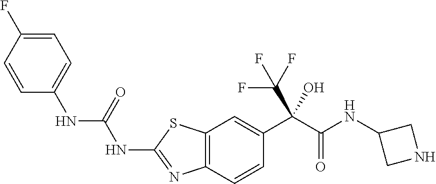 [SMILES: O=C(Nc1ccc(F)cc1)Nc1nc2ccc([C@](O)(C(=O)NC3CNC3)C(F)(F)F)cc2s1]

tert-Butyl (S)-3-(3,3,3-trifluoro-2-(2-(3-(4-fluorophenyl)ureido)benzo[d]thiazol-6-yl)-2-hydroxypropanamido)azetidine-1-carboxylate (0.07 g, 0.12 mmol) was dissolved in a solution of DCM (2 mL) and TFA (0.4 mL). Then it was stirred at 25° C. for 1 h. After completion, the reaction was diluted with sat. NaHCO₃ solution (5 mL) and extracted with EtOAc (5 mL×3). The combined organic layer was washed with brine, dried over Na₂SO₄, filtered and concentrated under reduced pressure to afford the title compound (0.02 g, 34.5%) as a yellow oil, which was directly used in the next step without further purification. MS (ESI): mass calcd, for C₂₀H₁₇F₄N₅O₃S: 483.10 found: 484.1 [M+H]+.

### Methyl (S)-3-(3,3,3-trifluoro-2-(2-(3-(4-fluorophenyl)ureido)benzo[d]thiazol-6-yl)-2-hydroxypropanamido)azetidine-1-carboxylate (366)

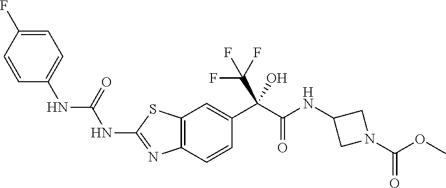 [SMILES: COC(=O)N1CC(NC(=O)[C@@](O)(c2ccc3nc(NC(=O)Nc4ccc(F)cc4)sc3c2)C(F)(F)F)C1]

To an ice-cold solution of (2S)—N-(azetidin-3-yl)-3,3,3-trifluoro-2-[2-[(4-fluorophenyl) carbamoylamino]-1,3-benzothiazol-6-yl]-2-hydroxy-propanamide (0.07 g, 0.14 mmol) in DCM (1 mL) was added DIPEA (76 μL, 0.43 mmol) and methyl carbonochloridate (13 μL, 0.17 mmol). Then it was allowed to stir at rt for 2 h under N₂. Upon completion, the reaction was diluted with sat. NH₄Cl solution (5 mL) and extracted with EtOAc (5 mL×3). The combined organic layer was washed with brine, dried over Na₂SO₄, filtered, and concentrated. The crude was purified by reverse phase HPLC (gradient separation, 30-60% ACN in H₂O with 0.1% TFA as a modifier) to afford the title compound (366) (0.017 g, 21.7%) as a white solid. MS (ESI): mass calcd, for C₂₂H₁₉F₄N₅O₅S: 541.10, found: 542.1 [M+H]⁺. ¹H NMR (400 MHz, DMSO-d6) δ ppm 10.95 (s, 1H), 9.23 (s, 1H), 8.89 (d, J=7.2 Hz, 1H), 8.14 (s, 1H), 7.90 (s, 1H), 7.59-7.68 (m, 2H), 7.51-7.57 (m, 2H), 7.17 (t, J=8.8 Hz, 2H), 4.47-4.55 (m, 1H), 4.00-4.13 (m, 2H), 3.91 (m, 1H), 3.74-3.81 (m, 1H), 3.53 (s, 3H).

Examples 383, 388, 401, 406, 410, 411, 413 (last step is alkylation of (2S)—N-(azetidin-3-yl)-3,3,3-trifluoro-2-[2-[(4-fluorophenyl) carbamoylamino]-1,3-benzothiazol-6-yl]-2-hydroxy-propanamide with 2-bromo-1,1-difluoroethane), 416, 417, 419, 422, 423 (last step is alkylation of (2S)—N-(azetidin-3-yl)-3,3,3-trifluoro-2-[2-[(4-fluorophenyl) carbamoylamino]-1,3-benzothiazol-6-yl]-2-hydroxy-propanamide with 2,2,2-trifluoroethyl trifluoromethanesulfonate), 424 (last step is alkylation of (2S)—N-(azetidin-3-yl)-3,3,3-trifluoro-2-[2-[(4-fluorophenyl) carbamoylamino]-1,3-benzothiazol-6-yl]-2-hydroxy-propanamide with 1-fluoro-2-iodoethane), 428, 429, 442 and 446 were synthesized in similar procedures as described in Example 366.

Example 367 was synthesized in similar procedures as described in Example 38, starting from the enantiomer intermediate from step 1 chiral SFC that is opposite of which that led to Example 100.

### Example 384. 2-Acetamido-N-ethyl-3,3,3-trifluoro-2-(2-(3-(4-fluorophenyl)ureido)benzo[d]thiazol-6-yl)propanamide (384)

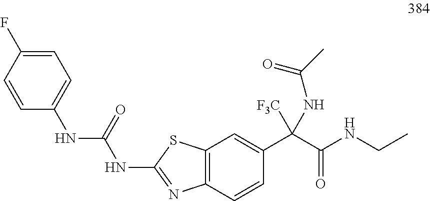 [SMILES: CCNC(=O)C(NC(C)=O)(c1ccc2nc(NC(=O)Nc3ccc(F)cc3)sc2c1)C(F)(F)F]

### Synthetic Scheme:

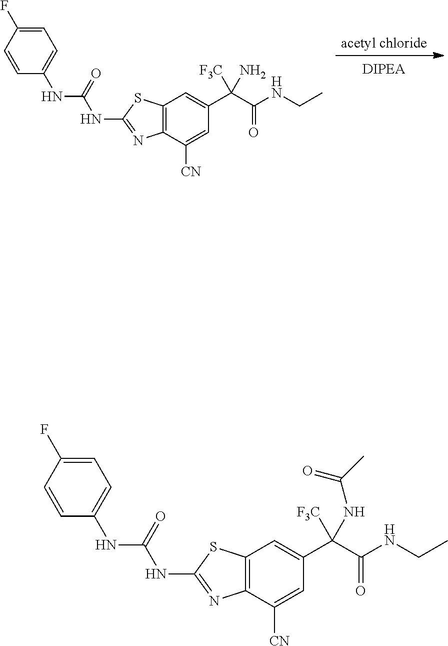 [SMILES: CCNC(=O)C(N)(c1cc(C#N)c2nc(NC(=O)Nc3ccc(F)cc3)sc2c1)C(F)(F)F ; "DIPEA" ; "acetyl chloride" ; CCNC(=O)C(NC(C)=O)(c1cc(C#N)c2nc(NC(=O)Nc3ccc(F)cc3)sc2c1)C(F)(F)F]

### 2-Acetamido-N-ethyl-3,3,3-trifluoro-2-(2-(3-(4-fluorophenyl)ureido)benzo[d]thiazol-6-yl)propenamide (384)

To a solution of 2-amino-N-ethyl-3,3,3-trifluoro-2-(2-(3-(4-fluorophenyl)ureido)benzo[d]thiazol-6-yl)propenamide (5.5 mg, 0.012 mmol) in THF (0.12 mL) was added acetyl chloride (4.3 μL, 0.06 mmol), followed by DIPEA (21 μL, 0.12 mmol).

The resulting solution was allowed to stir at rt for 1 h. After completion, the reaction mixture was diluted with ACN and water to 5 mL and it was then directly purified by reverse phase HPLC (gradient separation, 35-75% ACN in H₂O with 0.1% TFA as a modifier) to afford the title product (384) (1 mg, 17%, racemic) as a white solid. MS (ESI): mass calcd. for C₂₁H₁₉F₄N₅O₃S: 497.11 found: 498.10 [M+H]⁺. ¹H-NMR (400 MHz; MeOD) δ ppm 8.10 (s, 1H), 7.76 (d, J=8.7 Hz, 1H), 7.63 (dd, J=8.7, 1.5 Hz, 1H), 7.41 (dd, J=8.8, 4.8 Hz, 2H), 7.24 (t, J=8.6H), 3.19-3.26 (m, 2H), 2.02 (s, 3H), 1.04 (t, J=7.2 Hz, 3H).

Examples 243 and Example 385 were synthesized in similar procedures as described in Example 384. Example 243 was synthesized using N,N-dimethylchloroformylamine and (S)—N-ethyl-3,3,3-trifluoro-2-(2-(3-(4-fluorophenyl)ureido)benzo[d]thiazol-6-yl)-2-hydroxypropanamide.

Example 387 was synthesized by reacting (S)-2-(2-aminobenzo[d]thiazol-6-yl)-N-ethyl-3,3,3-trifluoro-2-hydroxypropanamide with 4-fluorophenyl carbonochloridate, TEA in DCM at 0° C. for 2 h.

### Example 389. (S)-3,3,3-Trifluoro-2-(2-(3-(4-fluorophenyl)ureido)benzo[d]thiazol-6-yl)-2-hydroxy-N-(3-(oxazol-2-yl)cyclobutyl)propanamide (389)

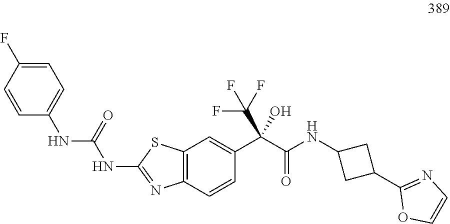 [SMILES: O=C(Nc1ccc(F)cc1)Nc1nc2ccc([C@](O)(C(=O)NC3CC(c4ncco4)C3)C(F)(F)F)cc2s1]

### Synthetic Scheme:

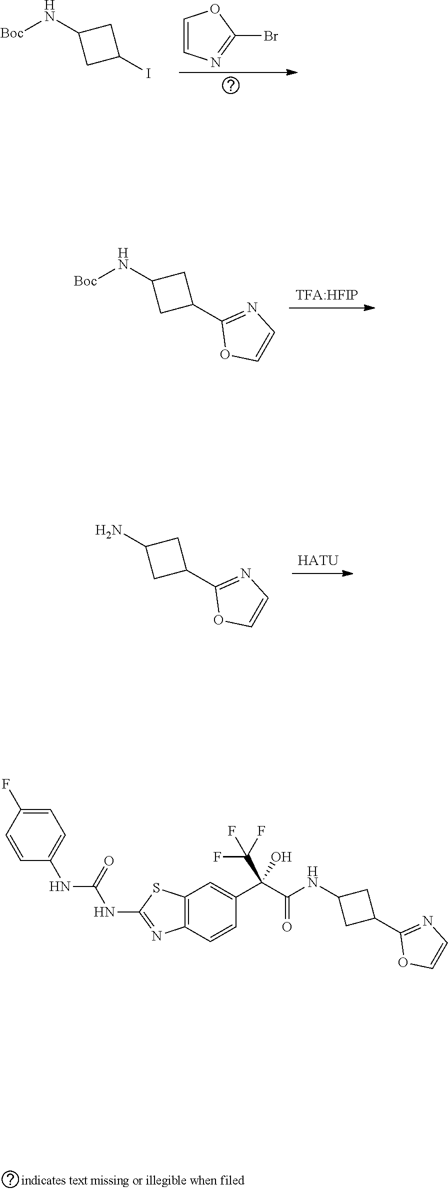

### tert-Butyl N-(3-oxazol-2-ylcyclobutyl)carbamate

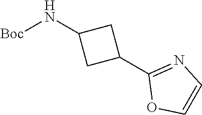 [SMILES: CC(C)(C)OC(=O)NC1CC(c2ncco2)C1]

To a solution of tert-butyl N-(3-iodocyclobutyl)carbamate (0.2 g, 0.67 mmol) in DME (2 mL) was added bis[3,5-difluoro-2-[5-(trifluoromethyl)-2-pyridyl]phenyl]iridium (1+);4-tert-butyl-2-(4-tert-butyl-2-pyridyl)pyridine;hexafluorophosphate (7.6 mg, 6.7 μmol), dichloronickel; 1,2-dimethoxyethane (0.74 mg, 3.4 μmol), Na₂CO₃ (143 mg, 1.35 mmol), dtbbpy (0.90 mg, 3.4 μmol), TTMSS (167.4 mg, 0.67 mmol) and 2-bromooxazole (149 mg, 1.01 mmol) at 25° C. The reaction was stirred under 34 W blue LED at 25° C. for 12 h. The reaction mixture was diluted with H₂O 15 mL and extracted with EtOAc 15 mL (5 mL×3). The combined organic layer was washed with brine 15 mL (5 ml×3), dried over Na₂SO₄, filtered and concentrated under reduced pressure. The residue was purified by reverse phase HPLC (H₂O) (0.2% FA)-ACN, gradient 25-60% elution) to provide the title compound (0.13 g, 81%) as a brown solid. MS (ESI): mass calcd, for C₁₂H₁₈N₂O₃: 238.13, found: 239.3 [M+H]⁺.

### 3-Oxazol-2-ylcyclobutanamine

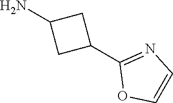

A solution of tert-butyl N-(3-oxazol-2-ylcyclobutyl)carbamate (0.13 g, 0.55 mmol) in HFIP (2 mL) and TFA (0.1 mL) was stirred at 25° C. for 12 h. The reaction was filtered and concentrated under reduced pressure to provide the title compound (0.2 g, crude, TFA salt) as a brown oil, which was used directly in the next step without further purification. MS (ESI): mass calcd, for C₇H₁₀N₂O: 138.08, found: 139.3 [M+H]⁺.

### (S)-3,3,3-Trifluoro-2-(2-(3-(4-fluorophenyl)ureido)benzo[d]thiazol-6-yl)-2-hydroxy-N-(3-(oxazol-2-yl)cyclobutyl)propanamide (389)

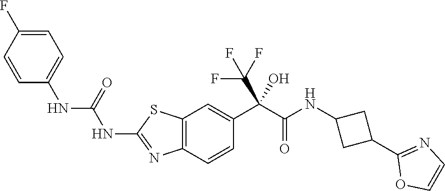 [SMILES: O=C(Nc1ccc(F)cc1)Nc1nc2ccc([C@](O)(C(=O)NC3CC(c4ncco4)C3)C(F)(F)F)cc2s1]

To a solution of (S)-3,3,3-trifluoro-2-(2-(3-(4-fluorophenyl)ureido)benzo[d]thiazol-6-yl)-2-hydroxypropanoic acid (0.1 g, 0.23 mmol) in DMF (5 mL) was added DIPEA (150.5 mg, 1.16 mmol) and HATU (177 mg, 0.47 mmol) at 0° C. After stirring at 25° C. for 15 min, 3-oxazol-2-ylcyclobutanamine (70.5 mg, crude, TFA salt) was added and the reaction was stirred at 25° C. for 2 h. The reaction was diluted with H₂O (15 mL) and extracted with EtOAc (10 mL×3). The combined organic layer was washed with brine, dried over Na₂SO₄, filtered and concentrated under reduced pressure. The residue was purified by reverse phase HPLC (H₂O (10 mM NH₄HCO₃)-ACN], gradient 30-60% elution) to provide the title compound (389) (3.2 mg, 2.5%, diastereomeric mixture) as a white solid. MS (ESI): mass calcd, for C₂₄H₁₉F₄N₅O₄S: 549.11, found: 550.1 [M+H]⁺. ¹H NMR (400 MHz, DMSO-d6) δ ppm 10.81 (s, 1H), 9.19 (s, 1H), 8.52-8.63 (m, 1H), 8.16 (s, 1H), 7.95-8.03 (m, 1H), 7.77-7.86 (m, 1H), 7.61-7.70 (m, 2H), 7.54 (d, J=3.6 Hz, 2H), 7.17 (t, J=8.8 Hz, 2H), 7.08-7.14 (m, 1H), 4.20-4.58 (m, 1H), 3.40-3.53 (m, 1H), 2.55-2.60 (m, 2H), 2.38-2.44 (m, 1H), 2.23-2.38 (m, 1H).

Examples 391 started from 2-(4-bromophenyl) propan-2-amine, followed by Cbz protection of the amine with Cbz-Cl and TEA, and then in similar procedures as described in Example 6 to make benzyl(2-(2-(3-(4-fluorophenyl)ureido)benzo[d]thiazol-6-yl) propan-2-yl)carbamate, which was deprotected with TBAF and followed by reaction with ethanesulfonyl chloride to afford 391.

Example 392 was synthesized from heating (2S)—N-(Azetidin-3-yl)-3,3,3-trifluoro-2-[2-[(4-fluorophenyl) carbamoylamino]-1,3-benzothiazol-6-yl]-2-hydroxy-propanamide (see Example 366 for synthesis), 2-chloropyrazine and DIPEA in EtOH at 90° C. for 6 h.

Example 393 was synthesized in similar procedures as described in Example 392, reacting with 2-fluoropyridine, DIPEA at 120° C. for 6 h.

Example 412 was synthesized in similar procedures as described in Example 392, reacting with 2-bromothiazole, TEA in DMF at 100° C. for 12 h.

Example 434 was synthesized in similar procedures as described in Example 392, reacting with 2-bromo-1,3,4-thiadiazole, and DIPEA in n-BuOH at 120° C. for 0.5 h.

### Example 399 and 400. (1R,2S)—N-Ethyl-2-((S)-3,3,3-trifluoro-2-(2-(3-(4-fluorophenylureido)benzo[d]thiazol-6-yl)-2-hydroxypropanamido)cyclobutane-1-carboxamide (399) and (1S,2R)—N-Ethyl-2-((S)-3,3,3-trifluoro-2-(2-(3-(4-fluorophenyl)ureido)benzo[d]thiazol-6-yl)-2-hydroxypropanamido)cyclobutane-1-carboxamide (400)

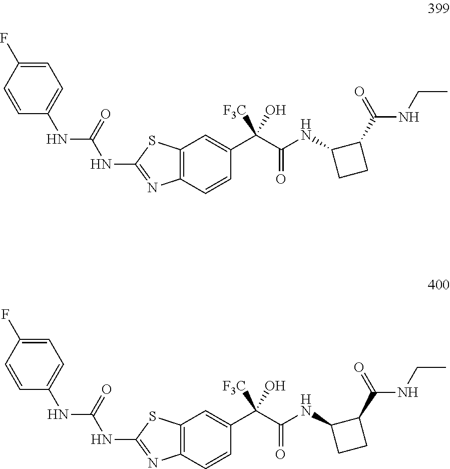 [SMILES: CCNC(=O)[C@@H]1CC[C@@H]1NC(=O)[C@@](O)(c1ccc2nc(NC(=O)Nc3ccc(F)cc3)sc2c1)C(F)(F)F ; CCNC(=O)[C@H]1CC[C@H]1NC(=O)[C@@](O)(c1ccc2nc(NC(=O)Nc3ccc(F)cc3)sc2c1)C(F)(F)F]

### Synthetic Scheme:

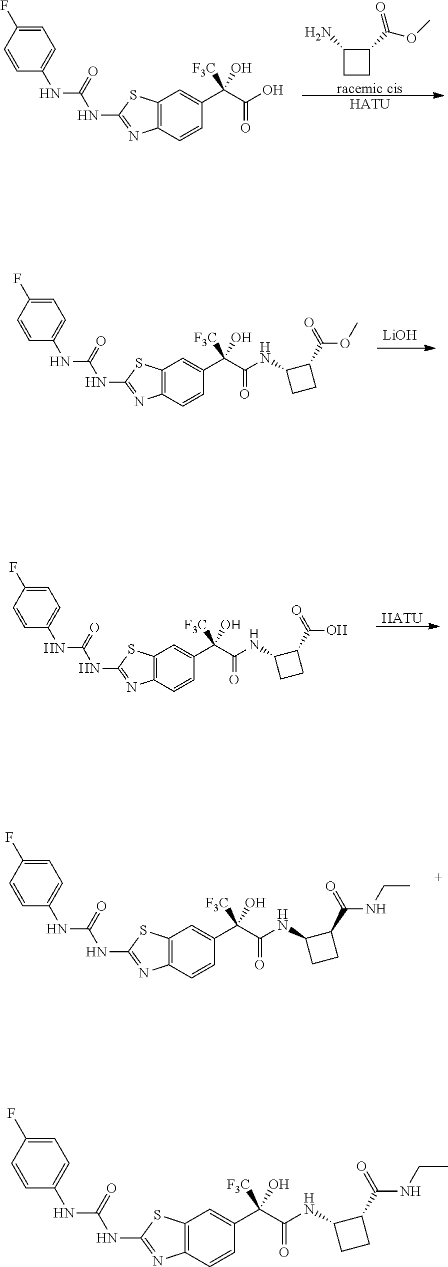

### Methyl 2-((S)-3,3,3-trifluoro-2-(2-(3-(4-fluorophenyl)ureido)benzo[d]thiazol-6-yl)-2-hydroxypropanamido)cyclobutane-1-carboxylate

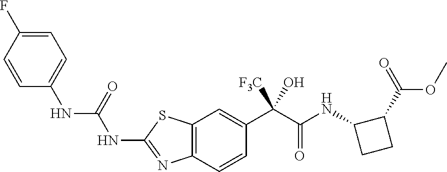 [SMILES: COC(=O)[C@@H]1CC[C@@H]1NC(=O)[C@@](O)(c1ccc2nc(NC(=O)Nc3ccc(F)cc3)sc2c1)C(F)(F)F]

To a solution of (S)-3,3,3-trifluoro-2-(2-(3-(4-fluorophenyl)ureido)benzo[d]thiazol-6-yl)-2-hydroxypropanoic acid (described in Example 38, 0.03 g, 0.07 mmol) in DMF (1 mL) was added DIPEA (61 μL, 0.035 mmol) and HATU (80 mg, 0.21 mmol) at 0° C. Rac-cis methyl 2-aminocyclobutane-1-carboxylate-trifluoroacetic acid (25.5 mg, 0.11 mmol) was added. After stirring at 20° C. for 2 h, the reaction was quenched with H₂O (2 mL) and extracted with EtOAc (2 mL×2). The combined organic layer was washed with brine, dried over Na₂SO₄, filtered, and concentrated under reduced pressure. The resulting residue was purified by silica gel column chromatography (gradient elution, 40-80% EtOAc in hexanes) to afford the title compound (12.5 mg, 33%, mixture of two diastereomers) as a colorless oil. MS (ESI): mass calcd, for C₂₃H₂₀F₄N₄O₅S: 540.11, found: 541.0 [M+H]⁺.

### 2-((S)-3,3,3-Trifluoro-2-(2-(3-(4-fluorophenyl)ureido)benzo[d]thiazol-6-yl)-2-hydroxypropanamido)cyclobutane-1-carboxylic Acid

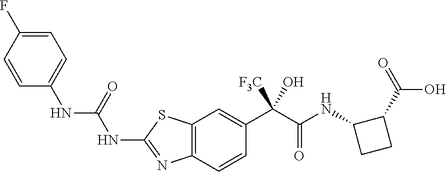 [SMILES: O=C(Nc1ccc(F)cc1)Nc1nc2ccc([C@](O)(C(=O)N[C@H]3CC[C@H]3C(=O)O)C(F)(F)F)cc2s1]

A suspension of methyl 2-((S)-3,3,3-trifluoro-2-(2-(3-(4-fluorophenyl)ureido)benzo[d]thiazol-6-yl)-2-hydroxypropanamido)cyclobutane-1-carboxylate (12.5 mg, 0.023 mmol) and LiOH·H₂O (1.7 mg, 0.07 mmol) in THF (0.4 mL), MeOH (0.1 mL), and H₂O (0.11 mL) was stirred at rt for 2 h. After completion, the reaction solvent was removed. The resulting crude residue was purified by reverse phase HPLC (gradient elution, 30-70% ACN in H₂O with 0.1% TFA as a modifier) to provide the title product (9.5 mg, 78%, mixture of two diastereomers) as a white solid. MS (ESI): mass calcd. for C₂₂H₁₈F₄N₄O₅S: 526.09, found: 527.2 [M+H]⁺.

### (1R,2S)—N-Ethyl-2-((S)-3,3,3-trifluoro-2-(2-(3-(4-fluorophenyl)ureido)benzo[d]thiazol-6-yl)-2-hydroxypropanamido)cyclobutane-1-carboxamide (399) and (1S,2R)—N-Ethyl-2-((S)-3,3,3-trifluoro-2-(2-(3-(4-fluorophenyl)ureido)benzo[d]thiazol-6-yl)-2-hydroxypropanamido)cyclobutane-1-carboxamide (400)

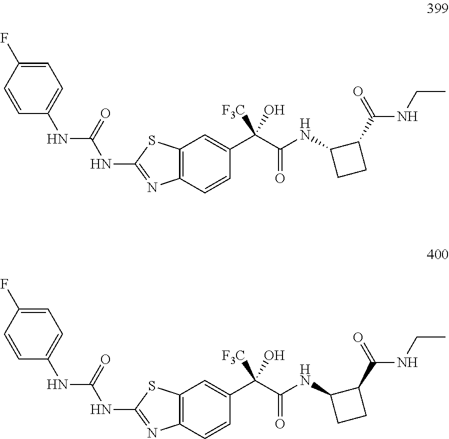 [SMILES: CCNC(=O)[C@@H]1CC[C@@H]1NC(=O)[C@@](O)(c1ccc2nc(NC(=O)Nc3ccc(F)cc3)sc2c1)C(F)(F)F ; CCNC(=O)[C@H]1CC[C@H]1NC(=O)[C@@](O)(c1ccc2nc(NC(=O)Nc3ccc(F)cc3)sc2c1)C(F)(F)F]

To a solution of 2-((S)-3,3,3-trifluoro-2-(2-(3-(4-fluorophenyl)ureido)benzo[d]thiazol-6-yl)-2-hydroxypropanamido)cyclobutane-1-carboxylic acid (9.5 mg, 0.018 mmol) in DMF (0.09 mL) was added DIPEA (9.5 μL, 0.054 mmol) and HATU (13.7 mg, 0.36 mmol) at 0° C. Ethyl amine hydrochloride (2.21 mg, 0.027 mmol) was added. After stirring at 20° C. for 30 minutes, the reaction was diluted with ACN and water. It was directly purified by reverse phase HPLC (gradient elution, 30-70% ACN in H₂O with 0.1% TFA as a modifier) to provide title compound (399) as a white solid. MS (ESI): mass calcd, for C₂₄H₂₃F₄N₅O₄S: 553.14, found: 554.20 [M+H]⁺, ¹H NMR (400 MHz; DMSO-d6) δ ppm 10.90 (br s, 1H), 9.22 (d, J=1.0 Hz, 1H), 8.17 (d, J=9.2 Hz, 1H), 8.09 (s, 1H), 7.98 (s, 1H), 7.74 (t, J=5.4 Hz, 1H), 7.63 (s, 2H), 7.54 (dd, J=8.2, 5.0 Hz, 2H), 7.17 (t, J=8.8 Hz, 2H), 4.54 (dt, J=17.2, 8.6 Hz, 1H), 3.10-3.15 (m, 1H), 2.78-2.89 (m, 1H), 2.67-2.76 (m, 1H), 2.18-2.27 (m, 2H), 1.81-1.94 (m, 2H), 0.70 (t, J=7.2 Hz, 3H) and the title compound (400) as a white solid, MS (ESI): mass calcd, for C₂₄H₂₃F₄N₅O₄S: 553.14, found: 554.20 [M+H]⁺, ¹H NMR (400 MHz; CD₃OD) δ ppm 8.36 (d, J=8.7 Hz, 1H), 8.10 (d, J=1.4 Hz, 1H), 7.68 (d, J=1.5 Hz, 1H), 7.60 (d, J=8.7 Hz, 1H), 7.44-7.48 (m, 2H), 7.03 (t, J=8.8 Hz, 2H), 4.55-4.63 (m, 1H), 3.16 (qd, J=7.3, 3.0 Hz, 2H), 2.15-2.27 (m, 2H), 1.94-1.99 (m, 2H), 1.07 (t, J=7.3 Hz, 3H). Stereochemistry was arbitrarily assigned.

Example 316 was synthesized in similar procedures as described in Example 399, with methyl (S)-azetidine-2-carboxylate hydrochloride used as the amine first, followed by LiOH-mediated hydrolysis and HATU coupling with azetidine.

Example 317, 323, 325, 326, 329, 330, 335, 378, 379, 380, 381 were synthesized in similar procedures as Example 399.

### Example 444. (S)-3,3,3-Trifluoro-2-(2-(3-(4-fluorophenyl)ureido)benzo[d]thiazol-6-yl)-2-hydroxy-N—(R)-2-oxoazetidin-3-yl)propanamide (444)

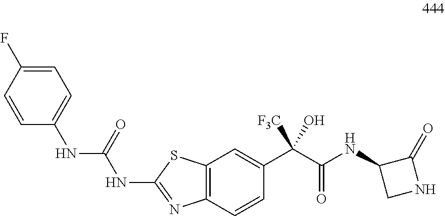 [SMILES: O=C(Nc1ccc(F)cc1)Nc1nc2ccc([C@](O)(C(=O)N[C@@H]3CNC3=O)C(F)(F)F)cc2s1]

### Synthetic Scheme:

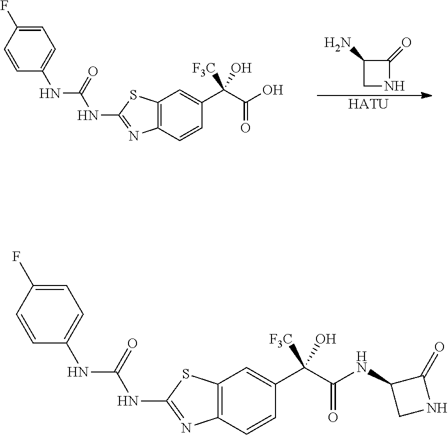

To a solution of (S)-3,3,3-trifluoro-2-(2-(3-(4-fluorophenyl)ureido)benzo[d]thiazol-6-yl)-2-hydroxypropanoic acid (described in Example 38, 0.03 g, 0.07 mmol) in DMF (0.35 mL) was added DIPEA (60.9 μL, 0.35 mmol) and HATU (79.7 mg, 0.21 mmol) at 0° C. Then (R)-3-aminoazetidin-2-one (20.4 mg, 0.28 mmol) was added. After stirring at 20° C. for 2 h, the reaction was quenched with H₂O (2 mL) and extracted with EtOAc (2 mL×2). The combined organic layer was washed with brine, dried over Na₂SO₄, filtered, and concentrated under reduced pressure. The resulting residue was purified by reverse phase HPLC (gradient elution, 30-80% ACN/H₂O, with 0.1% TFA as a modifier) to afford the title compound (444) (8.6 mg, 25%) as a white solid. MS (ESI): mass calcd, for C₂₀H₁₅F₄N₅O₄S: 497.08, found: 498.00 [M+H]⁺. ¹H NMR (400 MHz; DMSO-d6) δ ppm 10.97 (s, 1H), 9.23 (s, 1H), 8.86 (d, J=8.7 Hz, 1H), 8.17 (s, 1H), 7.94 (d, J=10.6 Hz, 2H), 7.67 (s, 2H), 7.52-7.56 (m, 2H), 7.17 (t, J=8.8 Hz, 2H), 4.85-4-89 (m, 1H), 3.48-3.52 (m, 1H), 3.21 (dd, J=5.0, 2.8 Hz, 1H).

Examples 242, 244, 247, 259, 260, 277, 283, 284, 285, 286, 289, 290, 297, 298, 299, 300, 302, 303, 305, 306, 309 and 310 (N-methylprop-2-en-1-amine was used as the amine first, followed by dihydroxylation with K₂S₂O₄, (DHQ) PHAL and K₃Fe(CN)₆ to make 309 and 310), 312 (methyl methylglycinate was used as the amine first, followed by reaction with MeMgBr, LiCl to form the final product), 315, 321, 322, 324, 327, 328, 332, 334 (methyl (S)-azetidine-2-carboxylate hydrochloride was used as amine first, followed by reaction with MeMgBr), 339, 340, 341, 343, 345, 346, 349, 355 (1-(pyrimidin-2-yl)azetidin-3-amine was used as the amine, which was prepared by alkylating tert-butyl azetidin-3-ylcarbamate HCl salt with 2-chloropyrimidine at 110° C. in NMP with DIPEA, followed by TF A/HFIP deprotection of the Boc group), 356, 357, 358, 359, 360, 361, 362, 368, 370, 375, 376, 377, 386, 394, 395 ((1s,3s)-3-amino-N-cyclopropylcyclobutane-1-carboxamide was used as the amine, which was prepared from (1s,3s)-3-((tert-butoxycarbonyl)amino)cyclobutane-1-carboxylic acid coupling with cyclopropyl amine using HATU followed by Boc deprotection with HCl in EtOAc), 396, 397, 398, 402, 403, 404, 407, 408, 409 ((1s,3s)-3-aminocyclobutyl ethylcarbamate was used as the amine, which was prepared from tert-butyl ((1s,3s)-3-hydroxycyclobutyl)carbamate reacting with isocyanatoethane, followed by Boc deprotection) 414, 418, 420, 425, 426, 427, 435, 436, 437, 438, 439, 440, 441, 443, 444, 445, 450, 451, 452, 453, 454, 457 and 458 were synthesized in similar procedures as described in Example 444. For some of these compounds, 2 eq of HATU and 3 eq of DIPEA in DMF (10 v) were used for the coupling.

Example 433 was synthesized from heating a mixture of (2S)—N-(Azetidin-3-yl)-3,3,3-trifluoro-2-[2-[(4-fluorophenyl) carbamoylamino]-1,3-benzothiazol-6-yl]-2-hydroxy-propanamide (see Example 366 for synthesis), 2-bromooxazole, Pd₂(dba)₃ (0.1 eq), Xantphos (0.12 eq) and t-BuONa (3 eq) in dioxane at 75° C. for 12 h.

### Example 449 N-Ethyl-3,3,3-trifluoro-2-(2-(3-(4-fluorophenyl)ureido)-4-hydroxybenzo[d]thiazol-6-yl)-2-hydroxypropanamide (449)

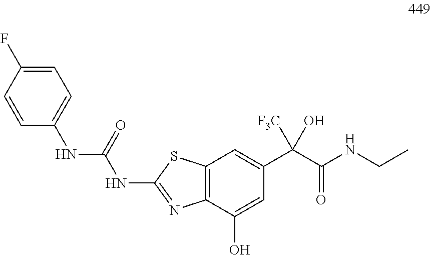 [SMILES: CCNC(=O)C(O)(c1cc(O)c2nc(NC(=O)Nc3ccc(F)cc3)sc2c1)C(F)(F)F]

### Synthetic Scheme:

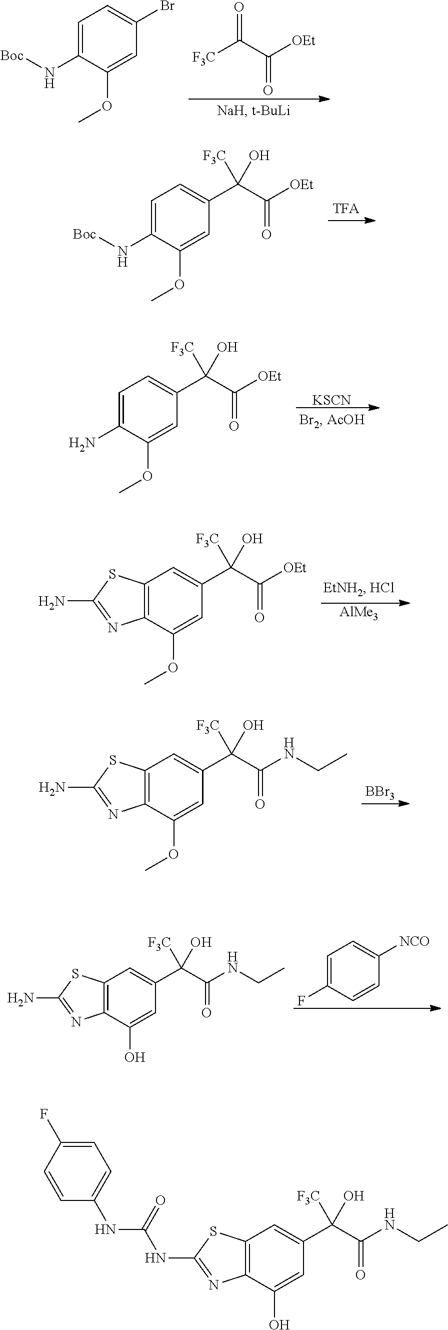

### Ethyl 2-(4-((tert-butoxycarbonyl)amino)-3-methoxyphenyl)-3,3,3-trifluoro-2-hydroxypropanoate

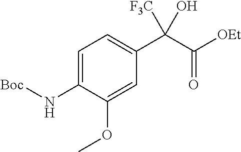 [SMILES: CCOC(=O)C(O)(c1ccc(NC(=O)OC(C)(C)C)c(OC)c1)C(F)(F)F]

To an ice-cold solution of tert-butyl N-(4-bromo-2-methoxy-phenyl)carbamate (20 g, 66.2 mmol) in THF (200 mL) was added 60% NaH (2.91 g, 72.8 mmol). After stirring at rt for 0.5 h, the reaction was cooled to −78° C., followed by dropwise addition of 1.3 M t-BuLi solution in pentane (101.8 mL, 132.4 mmol)). After stirring at −78° C. for 0.5 h, ethyl 3,3,3-trifluoro-2-oxo-propanoate (13.16 mL, 99.3 mmol) was added and stirring was continued for an additional hour at the same temperature. The reaction was quenched with H₂O (200 mL) and extracted with EtOAc (200 mL×3). The combined organic layer was washed with brine, dried over Na₂SO₄, filtered, and concentrated. The crude was purified by silica gel column chromatography (gradient elution, 0-100% EtOAc in PE) to afford the title compound (11 g, 42.3%) as a yellow oil. MS (ESI): mass calcd, for C₁₇H₂₂F₃NO₆: 393.14, found: 394.1 [M+H]⁺.

### Ethyl 2-(4-amino-3-methoxyphenyl)-3,3,3-trifluoro-2-hydroxypropanoate

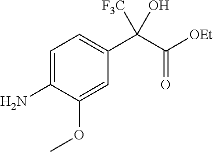 [SMILES: CCOC(=O)C(O)(c1ccc(N)c(OC)c1)C(F)(F)F]

Ethyl 2-(4-((tert-butoxycarbonyl)amino)-3-methoxyphenyl)-3,3,3-trifluoro-2-hydroxypropanoate (10 g, 25.4 mmol) was dissolved in a solution of DCM (80 mL) and TFA (16 mL). After stirring at 20° C. for 12 h, the reaction was poured into ice-water (100 mL), adjusted to pH=8 with Na₂CO₃ and extracted with DCM (100 mL×2). The combined organic layer was dried over Na₂SO₄, filtered, and concentrated. The crude was purified by silica gel column chromatography (gradient elution, 0-100% EtOAc in PE) to provide the title compound (7.5 g, with impurities) as a white solid. MS (ESI): mass calcd, for C₁₂H₁₄F₃NO₄: 293.09, found: 294.1 [M+H]⁺.

### 2-(2-Amino-4-methoxybenzo[d]thiazol-6-yl)-N-ethyl-3,3,3-trifluoro-2-hydroxypropanamide

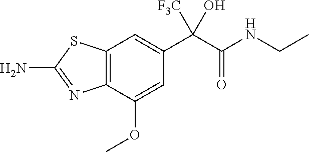 [SMILES: CCNC(=O)C(O)(c1cc(OC)c2nc(N)sc2c1)C(F)(F)F]

The title compound was synthesized from ethyl 2-(4-amino-3-methoxy-phenyl)-3,3,3-trifluoro-2-hydroxy-propanoate in similar procedures as described in Example 61. MS (ESI): mass calcd, for C₁₃H₁₄F₃N₃O₃S: 349.07, found: 350.1 [M+H]⁺.

### 2-(2-Amino-4-hydroxybenzo[d]thiazol-6-yl)-N-ethyl-3,3,3-trifluoro-2-hydroxypropanamide

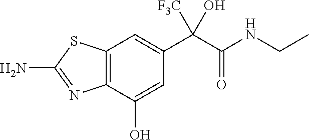 [SMILES: CCNC(=O)C(O)(c1cc(O)c2nc(N)sc2c1)C(F)(F)F]

To a solution of 2-(2-amino-4-methoxybenzo[d]thiazol-6-yl)-N-ethyl-3,3,3-trifluoro-2-hydroxypropanamide (2.1 g, 6.0 mmol) in DCM (20 mL) was added BBr₃ (9.04 g, 36.1 mmol). After stirring at 20° C. for 12 h, the reaction was poured into water (50 mL), adjusted to pH=8 with Na₂CO₃ and extracted with EtOAc (50 mL×3). The combined organic layer was dried over Na₂SO₄, filtered, and concentrated. The crude was purified by silica gel column chromatography (gradient elution, 0-100% EtOAc in PE) to provide the title compound (1.45 g, 71.9%) as a yellow solid. MS (ESI): mass calcd, for C₁₂H₁₂F₃N₃O₄S: 335.06, found: 336.0 [M+H]⁺.

### N-Ethyl-3,3,3-trifluoro-2-(2-(3-(4-fluorophenyl)ureido)-4-hydroxybenzo[d]thiazol-6-yl)-2-hydroxypropanamide (449)

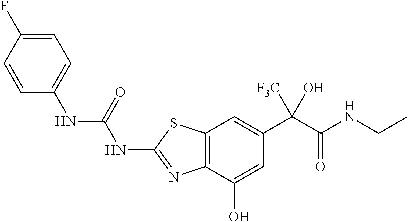 [SMILES: CCNC(=O)C(O)(c1cc(O)c2nc(NC(=O)Nc3ccc(F)cc3)sc2c1)C(F)(F)F]

To an ice-cold solution of 2-(2-amino-4-hydroxy-1,3-benzothiazol-6-yl)-N-ethyl-3,3,3-trifluoro-2-hydroxy-propanamide (1.4 g, 4.18 mmol) in DMF (14 mL) was added 1-fluoro-4-isocyanato-benzene (1.03 mL, 9.19 mmol) and warmed to rt. After stirring for 12 h, the reaction was washed with H₂O (42 mL) and extracted with EtOAc (42 mL×3). The combined organic layer was washed with brine, dried over Na₂SO₄, filtered, and concentrated. The crude was purified by reverse phase HPLC (gradient elution, 28-58% ACN/H₂O, with 10 mM NH₄HCO₃ as a modifier) to afford the title compound (449) (744 mg, 37.7%, racemic) as a white solid. MS (ESI): mass calcd, for C₁₉H₁₆F₄N₄O₄S: 472.08, found: 473.1 [M+H]⁺. ¹H NMR (400 MHz, DMSO-d₆) δ ppm 10.82 (s, 1H), 9.92 (s, 1H), 9.27 (s, 1H), 8.16 (t, J=6.0 Hz, 1H), 7.65 (s, 1H), 7.48-7.60 (m, 3H), 7.14-7.22 (m, 3H), 3.07-3.18 (m, 2H), 1.00 (t, J=7.2H).

### Example 460. 2-(2-((1H-Benzo[d]imidazol-2-yl)amino)benzo[d]thiazol-6-yl)-N-ethyl-3,3,3-trifluoro-2-hydroxypropanamide (460)

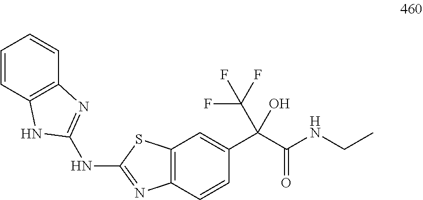 [SMILES: CCNC(=O)C(O)(c1ccc2nc(Nc3nc4ccccc4[nH]3)sc2c1)C(F)(F)F]

### Synthetic Scheme:

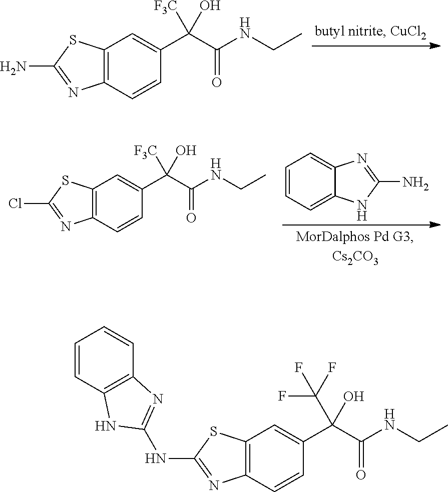

### 2-(2-Chlorobenzo[d]thiazol-6-yl)-N-ethyl-3,3,3-trifluoro-2-hydroxypropanamide

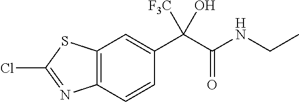 [SMILES: CCNC(=O)C(O)(c1ccc2nc(Cl)sc2c1)C(F)(F)F]

To a solution of CuCl₂ (505.3 mg, 3.76 mmol) in ACN (10 mL) was added ter-butyl nitrite (447 μL, 3.76 mmol) at 40° C. After stirring for 10 minutes, a solution of 2-(2-amino-1,3-benzothiazol-6-yl)-N-ethyl-3,3,3-trifluoro-2-hydroxy-propanamide (1 g, 3.13 mmol) in ACN (2 mL) was added dropwise. Then it was allowed to stir at 40° C. for an additional 2 h. Upon completion, the reaction mixture was filtered, and the filtrate was concentrated under reduced pressure. The resulting residue was purified by silica gel column chromatography (gradient elution, 0-100% EtOAc in PE) to afford the title compound (0.74 g, 69.8%) as a white solid. MS (ESI): mass calcd, for C₁₂H₁₀ClF₃N₂O₂S: 338.01, found: 339.1 [M+H]⁺.

### 2-(2-((1H-Benzo[d]imidazol-2-yl)amino)benzo[d]thiazol-6-yl)-N-ethyl-3,3,3-trifluoro-2-hydroxypropanamide

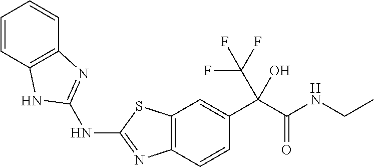 [SMILES: CCNC(=O)C(O)(c1ccc2nc(Nc3nc4ccccc4[nH]3)sc2c1)C(F)(F)F]

MorDalphos Pd G3 (24.6 mg, 29.5 μmol) and Cs₂CO₃ (192.4 mg, 0.59 mmol) were added to a solution of 2-(2-chloro-1,3-benzothiazol-6-yl)-N-ethyl-3,3,3-trifluoro-2-hydroxy-propanamide (100 mg, 0.30 mmol) and 1H-benzimidazol-2-amine (47.2 mg, 0.35 mmol) in dioxane (2 mL) under N₂. After heating at 90° C. for 12 h, the reaction was filtered through a celite pad. The filtrate was concentrated under reduced pressure. The resulting residue was purified by reverse phase HPLC (gradient elution, 35-60% ACN/H₂O, with 10 mM NH₄HCO₃ as a modifier) to afford the title compound (460) (26.2 mg, 20.3%, racemic) as a white solid. MS (EST): mass calcd, for C₁₉H₁₆F₃N₅O₂S: 435.10, found: 436.1 [M+H]⁺. ¹H NMR (400 MHz, DMSO-d6) δ ppm 8.47 (s, 1H), 8.30 (s, 1H), 8.06 (d, J=8.4 Hz, 1H), 7.86 (d, J=8.8 Hz, 1H), 7.79 (d, J=8.0 Hz, 1H), 7.72 (br s, 2H), 7.32 (d, J=8.0 Hz, 1H), 7.20 (t, J=7.6 Hz, 1H), 7.14 (t, J=8.0 Hz, 1H), 3.11-3.17 (m, 2H), 1.00 (t, J=7.2 Hz, 3H).

Examples 463 was synthesized in similar procedures as described in Example 460.

### Example 461. (2S)—N-Ethyl-2-[2-[(5-ethylthiazol-2-yl)amino]-1,3-benzothiazol-6-yl]-3,3,3-trifluoro-2-hydroxy-propanamide (461) and (2R)—N-Ethyl-2-[2-[(5-ethylthiazol-2-yl)amino]-1,3-benzothiazol-6-yl]-3,3,3-trifluoro-2-hydroxy-propanamide (462)

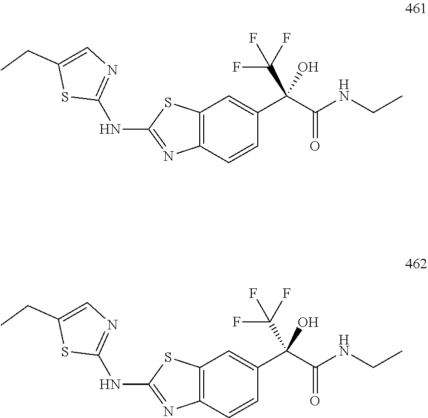

### Synthetic Scheme:

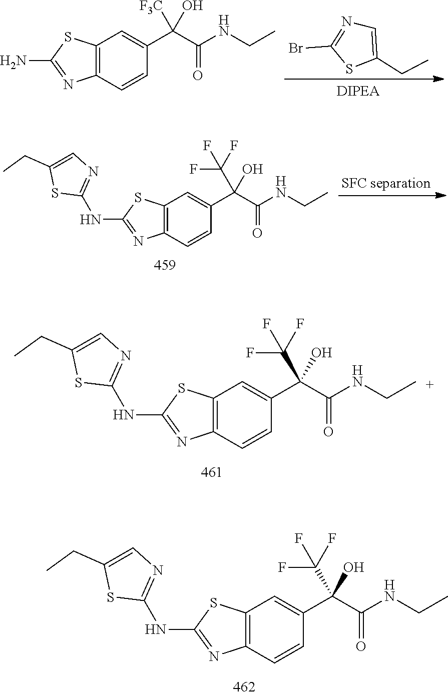

### N-Ethyl-2-(2-((S-ethylthiazol-2-yl)amino)benzo[d]thiazol-6-yl)-3,3,3-trifluoro-2-hydroxypropanamide (459)

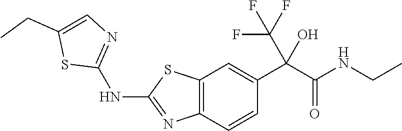 [SMILES: CCNC(=O)C(O)(c1ccc2nc(Nc3ncc(CC)s3)sc2c1)C(F)(F)F]

To a solution of 2-(2-amino-1,3-benzothiazol-6-yl)-N-ethyl-3,3,3-trifluoro-2-hydroxy-propanamide (100 mg, 0.31 mmol) in dioxane (2 mL) was added Cs₂CO₃ (204 mg, 0.63 mmol), 1,10-phenanthroline (5.6 mg, 31 μmol), (Bu₄NCuI) 2 (54.1 mg, 48 μmol) and 2-bromo-5-ethyl-thiazole (72.2 mg, 0.38 mmol). After heating at 120° C. for 12 h, the reaction was filtered, and the filtrate was concentrated under reduced pressure. The resulting residue was purified by reverse phase HPLC (gradient elution, 30-60% ACN/H₂O, with 10 mM NH₄HCO₃ as a modifier) to provide the title compound (459) (22 mg, 16.3%, racemic) as a white solid. MS (ESI): mass calcd, for C₁₇H₁₇F₃N₄O₂S₂: 430.07, found: 431.1 [M+H]⁺. ¹H NMR (400 MHz, DMSO-d₆) δ ppm 9.98 (s, 1H), 8.67 (d, J=8.8 Hz, 1H), 8.24 (s, 1H), 7.82 (s, 2H), 7.58-7.62 (m, 1H), 7.43 (s, 1H), 3.09-3.14 (m, 2H), 2.81-2.87 (m, 2H), 1.28 (t, J=7.2 Hz, 3H), 0.99 (t, J=7.2 Hz, 3H).

### (2S)—N-Ethyl-2-[2-[(5-ethylthiazol-2-yl)amino]-1,3-benzothiazol-6-yl]-3,3,3-trifluoro-2-hydroxy-propanamide (461) and (2R)—N-Ethyl-2-[2-[(5-ethylthiazol-2-yl)amino]-1,3-benzothiazol-6-yl]-3,3,3-trifluoro-2-hydroxy-propanamide (462)

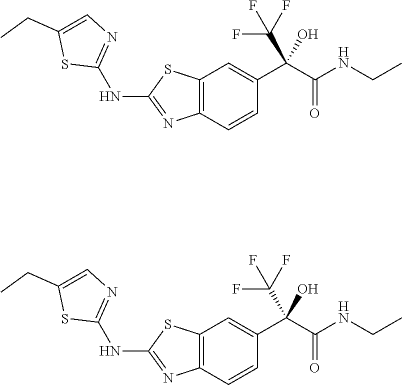

Example 459 was separated by chiral SFC (column: DAICEL CHIRALPAK IG (250 mm*30 mm, 10 μm); mobile phase: 45% MeOH (0.1% NH₃H₂O) isocratic elution) to afford the title compound (461) (4.5 mg, 28.1%) as a white solid. MS (ESI): mass calcd. for C₁₇H₁₇F₃N₄O₂S₂: 430.07, found: 431.1 [M+H]⁺. ¹H NMR (400 MHz, DMSO-d₆) δ ppm 9.98 (s, 1H), 8.66 (d, J=8.8 Hz, 1H), 8.24 (s, 1H), 7.81 (d, J=1.6 Hz, 2H), 7.58-7.61 (m, 1H), 7.43 (s, 1H), 3.09-3.15 (m, 2H), 2.81-2.87 (m, 2H), 1.27 (t, J=7.6 Hz, 3H), 0.99 (t, J=7.2 Hz, 3H). And to afford the title compound (462) (5.8 mg, 36.3%) as a white solid, MS (ESI): mass calcd, for C₁₇H₁₇F₃N₄O₂S₂: 430.07, found: 431.1 [M+H]⁺, ¹H NMR (400 MHz, DMSO-d₆) δ ppm 9.97 (s, 1H), 8.66 (d, J=8.8 Hz, 1H), 8.24 (s, 1H), 7.81 (d, J=1.6 Hz, 2H), 7.58-7.61 (m, 1H), 7.43 (s, 1H), 3.09-3.15 (m, 2H), 2.81-2.87 (m, 2H), 1.27 (t, J=7.6 Hz, 3H), 0.99 (t, J=7.2 Hz, 3H). Stereochemistry was arbitrarily assigned.

Examples 464 was synthesized in similar procedures as described in Example 459.

### Example 465. (S)—N-Ethyl-3,3,3-trifluoro-2-(2-((2-((4-fluorophenyl)amino)-3,4-dioxocyclobut-1-en-1-yl)amino)benzo[d]thiazol-6-yl)-2-hydroxypropanamide (465)

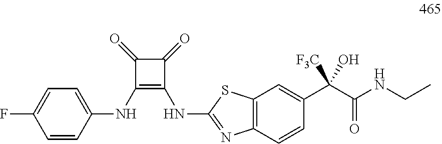 [SMILES: CCNC(=O)[C@@](O)(c1ccc2nc(Nc3c(Nc4ccc(F)cc4)c(=O)c3=O)sc2c1)C(F)(F)F]

### Synthetic Scheme:

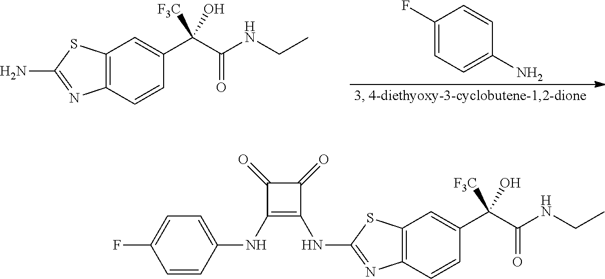 [SMILES: CCNC(=O)[C@@](O)(c1ccc2nc(N)sc2c1)C(F)(F)F ; Nc1ccc(F)cc1 ; CCNC(=O)[C@@](O)(c1ccc2nc(Nc3c(Nc4ccc(F)cc4)c(=O)c3=O)sc2c1)C(F)(F)F]

### (S)—N-Ethyl-3,3,3-trifluoro-2-(2-((2-((4-fluorophenyl)amino)-3,4-dioxocyclobut-1-en-1-yl)amino)benzo[d]thiazol-6-yl)-2-hydroxypropanamide (465)

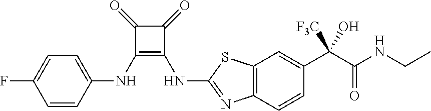 [SMILES: CCNC(=O)[C@@](O)(c1ccc2nc(Nc3c(Nc4ccc(F)cc4)c(=O)c3=O)sc2c1)C(F)(F)F]

(S)-2-(2-aminobenzo[d]thiazol-6-yl)-N-ethyl-3,3,3-trifluoro-2-hydroxypropanamide (described in Example 91, 20 mg, 0.063 mmol), 3,4-diethoxy-3-cyclobutene-1,2-dione (9.0 μL, 0.063 mmol), and TEA (8.7 μL. 0.063 mmol) were dissolved in EtOH (0.63 mL). After heating at 100° C. for 16 h, the reaction solvent was removed under reduced pressure. The resulting residue was purified by reverse phase HPLC (gradient elution, 20-60% ACN in H₂O with 0.1% TFA as a modifier) to provide title product (465). MS (ESI): mass calcd. for C₂₂H₁₆F₄N₄O₄S: 508.08, found: 508.80 [M+H]⁺. ¹H NMR (400 MHz: DMSO-d6) δ ppm 11.10 (s, 1H), 8.24 (t, J=5.9 Hz, 2H), 7.86 (s, 2H), 7.73 (d, J=8.8 Hz, 1H), 7.57-7.60 (m, 2H), 7.25 (t, J=8.7 Hz, 3H), 3.09-3.18 (m, 2H), 0.99 (t, J=7.1 Hz, 3H).

### Example 466. N-Ethyl-3,3,3-trifluoro-2-(4-((2-((4-fluorophenyl)amino)pyrimidin-4-yl)amino)phenyl)-2-hydroxypropanamide (466)

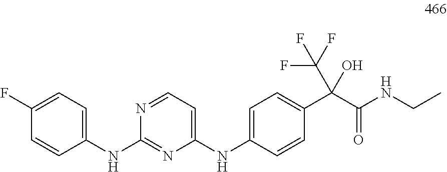 [SMILES: CCNC(=O)C(O)(c1ccc(Nc2ccnc(Nc3ccc(F)cc3)n2)cc1)C(F)(F)F]

### Synthetic Scheme:

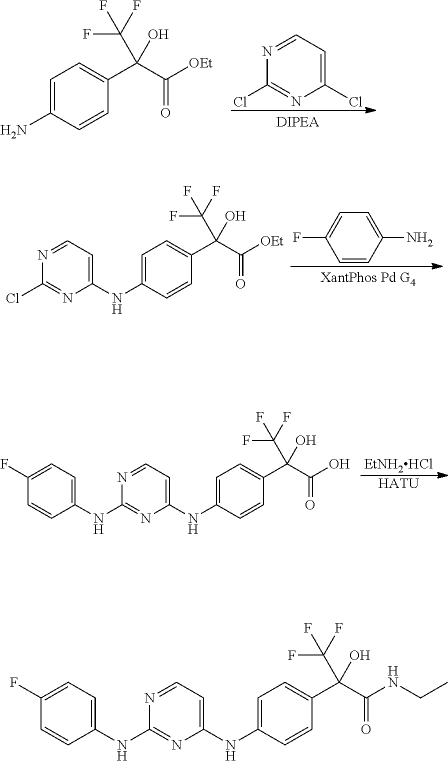

### Ethyl (2S)-2-[4-[(2-chloropyrimidin-4-yl)amino]phenyl]-3,3,3-trifluoro-2-hydroxy-propanoate

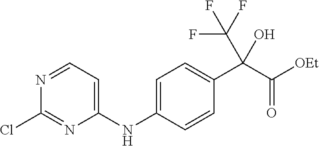 [SMILES: CCOC(=O)C(O)(c1ccc(Nc2ccnc(Cl)n2)cc1)C(F)(F)F]

To a solution of ethyl(2S)-2-(4-aminophenyl)-3,3,3-trifluoro-2-hydroxy-propanoate (0.2 g, 0.76 mmol) and 2,4-dichloropyrimidine (226.4 mg, 1.52 mmol) in DMF (2 mL) was added DIPEA (196.4 mg, 1.52 mmol). After heating at 100° C. for 12 h, the reaction was diluted with H₂O (5 mL) and extracted with EtOAc (10 mL×3). The combined organic layer was washed with brine, dried over Na₂SO₄, filtered and concentrated under reduced pressure. The residue was purified by silica gel column chromatography (Petroleum ether:Ethyl acetate=10:1 to 1:1) to provide the title compound (175 mg, 61%) as a yellow solid. MS (ESI): mass calcd, for C₁₅H₁₃ClF₃N₅O₃: 375.06, found: 376.0 [M+H]⁺.

### 3,3,3-Trifluoro-2-[4-[[2-(4-fluoroanilino)pyrimidin-4-yl]amino]phenyl]-2-hydroxy-propanoic Acid

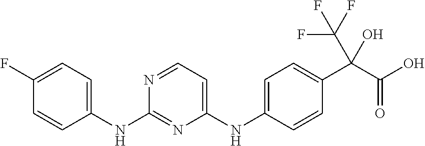 [SMILES: O=C(O)C(O)(c1ccc(Nc2ccnc(Nc3ccc(F)cc3)n2)cc1)C(F)(F)F]

To a solution of ethyl (2S)-2-[4-[(2-chloropyrimidin-4-yl)amino]phenyl]-3,3,3-trifluoro-2-hydroxy-propanoate (120 mg, 0.32 mmol) and 4-fluoroaniline (42.6 mg, 0.38 mmol) in dioxane (3 mL) was added (5-diphenylphosphanyl-9,9-dimethyl-xanthen-4-yl)-diphenyl-phosphane;methanesulfonate; [2-[2-(methylamino)phenyl]phenyl]palladium (1⁺) (XantPhos, 30.7 mg, 32 μmol) and Cs₂CO₃ (208 mg, 0.64 mmol). After heating at 90° C. for 12 h, the reaction was diluted with H₂O (5 mL) and extracted with EtOAc (5 mL×3). The combined organic layer was washed with brine, dried over Na₂SO₄, filtered and concentrated under reduced pressure. The residue was purified by prep-TLC (SiO₂, Petroleum ether:Ethyl acetate=1:1) to provide the title compound (33 mg, 24.5%) as a white solid. MS (ESI): mass calcd, for C₁₉H₁₄F₄N₄O₃: 422.10, found: 423.1 [M+H]⁺.

### N-Ethyl-3,3,3-trifluoro-2-(4-((2-(4-fluorophenyl)amino)pyrimidin-4-ylamino)phenyl)-2-hydroxypropanamide (466)

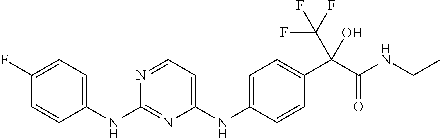 [SMILES: CCNC(=O)C(O)(c1ccc(Nc2ccnc(Nc3ccc(F)cc3)n2)cc1)C(F)(F)F]

To solution of 3,3,3-trifluoro-2-[4-[[2-(4-fluoroanilino) a pyrimidin-4-yl]amino]phenyl]-2-hydroxy-propanoic acid (33 mg, 78 μmol) and ethanamine hydrochloride (7.7 mg, 94 μmol) in DMF (2 mL) was added HATU (59.4 mg, 0.16 mmol) and DIPEA (30.3 mg, 0.23 mmol). After stirring at 25° C. for 2 h, the reaction was diluted with H₂O (5 mL) and extracted with EtOAc (5 mL×3). The combined organic layer was washed with brine, dried over Na₂SO₄, filtered and concentrated under reduced pressure. The residue was purified by reverse phase HPLC (H₂O (10 mM NH₄HCO₃)-ACN), gradient 30-60% elution) to provide the title compound (466) (6.5 mg, 19%) as a white solid. MS (ESI): mass calcd, for C₂₁H₁₉F₄N₅O₂: 449.15, found: 450.1 [M+H]⁺. ¹H NMR (400 MHz, DMSO-d₆) δ ppm 9.47 (s, 1H), 9.18 (s, 1H), 8.20 (t, J=5.6 Hz, 1H), 8.02 (d, J=5.6 Hz, 1H), 7.70-7.74 (m, 4H), 7.57-7.59 (m, 3H), 7.06 (t, J=8.8 Hz, 2H), 6.23 (d, J=5.6 Hz, 1H), 3.10-3.17 (m, 2H), 1.01 (t, J=7.2 Hz, 3H).

### Example 467. 4-(4-(3-(Ethylamino)-1,1,1-trifluoro-2-hydroxy-3-oxopropan-2-yl)phenyl)-N-phenyl-1H-imidazole-2-carboxamide (467)

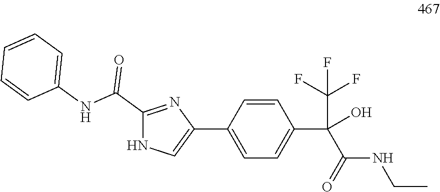 [SMILES: CCNC(=O)C(O)(c1ccc(-c2c[nH]c(C(=O)Nc3ccccc3)n2)cc1)C(F)(F)F]

### Synthetic Scheme:

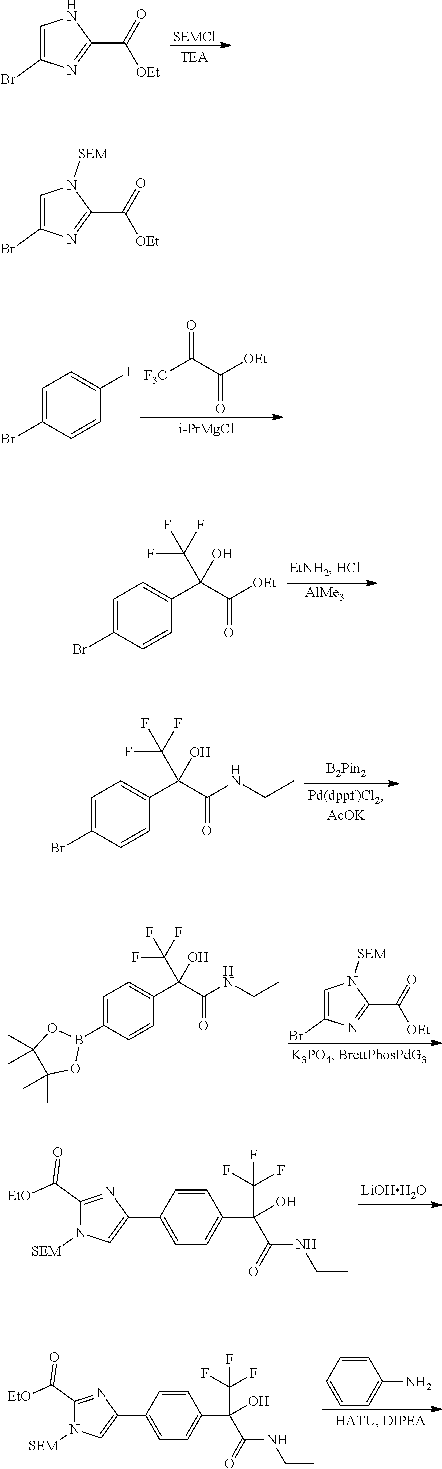

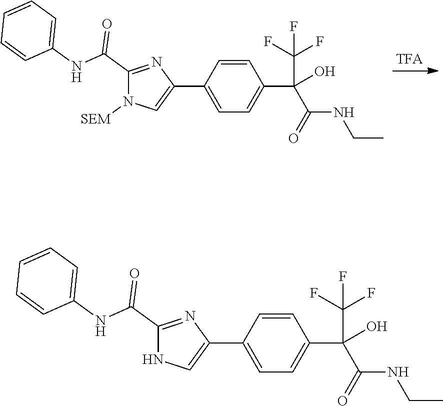 [SMILES: CCNC(=O)C(O)(c1ccc(-c2cn(COCC[Si](C)(C)C)c(C(=O)Nc3ccccc3)n2)cc1)C(F)(F)F ; CCNC(=O)C(O)(c1ccc(-c2c[nH]c(C(=O)Nc3ccccc3)n2)cc1)C(F)(F)F]

### Ethyl 4-bromo-1-((2-(trimethylsilyl)ethoxy)methyl)-1H-imidazole-2-carboxylate

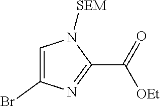 [SMILES: CCOC(=O)c1nc(Br)cn1COCC[Si](C)(C)C]

To a solution of ethyl 4-bromo-1H-imidazole-2-carboxylate (2 g, 9.13 mmol) in THE (20 mL) was added SEM-Cl (1.94 mL, 11 mmol) and TEA (2.54 mL, 18.3 mmol). After stirring at 25° C. for 12 h, the reaction was poured into H₂O (40 mL) and extracted with EtOAc (40 mL×3). The combined organic layer was washed with brine, dried with anhydrous Na₂SO₄, filtered and concentrated. The crude was purified by silica gel column chromatography (gradient elution, 6-11% EtOAc in PE) to afford the title compound (2.3 g, 72.1%) as a colorless oil. MS (ESI): mass calcd, for C₁₂H₂₁BrN₂O₃Si: 348.05, found: 349.0 [M+H]⁺.

### Ethyl 2-(4-bromophenyl)-3,3,3-trifluoro-2-hydroxypropanoate

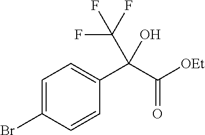 [SMILES: CCOC(=O)C(O)(c1ccc(Br)cc1)C(F)(F)F]

A solution of 2 M i-PrMgCl in THF (19.44 mL, 38.9 mmol) was added to a solution of 1-bromo-4-iodo-benzene (10 g, 35.4 mmol) in THF (100 mL) dropwise at −70° C. After 0.5 b, ethyl 3,3,3-trifluoro-2-oxo-propanoate (5.6 mL, 42.4 mmol) was added dropwise. The reaction was further stirred at −70° C. for 2 b. Upon completion, the reaction was quenched with sat. NH₄Cl (100 mL) and extracted with EtOAc (100 mL×3). The combined organic layer was washed with brine, dried over Na₂SO₄, filtered and concentrated. The crude was purified by silica gel column chromatography (gradient elution, 1-17% EtOAc in PE) to afford the title compound (8 g, 69.2%) as a colorless oil. MS (ESI): mass calcd, for C₁₁H₁₀BrF₃O₂: 325.98, found: 325.0 [M−H]⁺.

### 2-(4-Bromophenyl)-N-ethyl-3,3,3-trifluoro-2-hydroxypropanamide

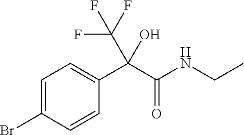 [SMILES: CCNC(=O)C(O)(c1ccc(Br)cc1)C(F)(F)F]

To an ice-cold solution of ethanamine hydrochloride (5.98 g, 73.4 mmol) in toluene (200 mL) was added a solution of 2 M AlMe₃ in toluene (36.7 mL, 110.1 mmol) dropwise. Then it was stirred at 40° C. for 1 h, followed by addition of ethyl 2-(4-bromophenyl)-3,3,3-trifluoro-2-hydroxy-propanoate (8 g, 24.5 mmol). After heating at 80° C. for 12 h, the reaction was quenched with sat. NH₄Cl (200 mL) and extracted with EtOAc (200 mL×3). The combined organic layer was washed with brine, dried over Na₂SO₄, filtered and concentrated. The crude was purified by silica gel column chromatography (gradient elution, 9-50% EtOAc in PE) to afford the title compound (6 g, 75.2%) as an off-white solid. MS (ESI): mass calcd. for C₁₁H₁₁BrF₃NO₂: 324.99, found: 325.9 [M+H]⁺.

### N-Ethyl-3,3,3-trifluoro-2-hydroxy-2-(4-(4,4,5,5-tetramethyl-1,3,2-dioxaborolan-2-yl)phenyl)propenamide

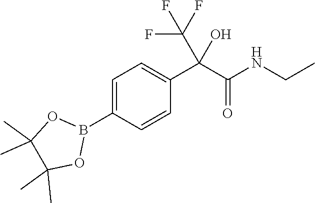 [SMILES: CCNC(=O)C(O)(c1ccc(B2OC(C)(C)C(C)(C)O2)cc1)C(F)(F)F]

To a solution of 2-(4-bromophenyl)-N-ethyl-3,3,3-trifluoro-2-hydroxypropanamide (5 g, 15.3 mmol) in dioxane (10 mL) was added Pd(dppf)Cl₂·CH₂Cl₂ (1.25 g, 1.53 mmol), B₂Pin₂ (4.67 g, 18.4 mmol) and AcOK (4.51 g, 46 mmol) under N₂. The resulting suspension was stirred at 80° C. for 12 h. Upon completion, the reaction was filtered through a celite pad. The filtrate was concentrated and purified by silica gel column chromatography (gradient elution, 9-50% EtOAc in PE) to afford the title compound (3.4 g, 59.4%) as a brown oil. MS (ESI): mass calcd, for C₁₇H₂₃BF₃NO₄: 373.17, found: 374.1 [M+H]⁺.

### Ethyl 4-(4-(3-(ethylamino)-1,1,1-trifluoro-2-hydroxy-3-oxopropan-2-yl)phenyl)-1-((2-(trimethylsilyl)ethoxy)methyl)-1H-imidazole-2-carboxylate

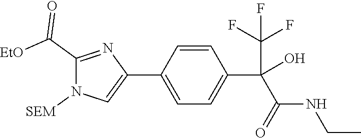 [SMILES: CCNC(=O)C(O)(c1ccc(-c2cn(COCC[Si](C)(C)C)c(C(=O)OCC)n2)cc1)C(F)(F)F]

A suspension of N-ethyl-3,3,3-trifluoro-2-hydroxy-2-[4-(4,4,5,5-tetramethyl-1,3,2-dioxaborolan-2-yl)phenyl]propanamide (200 mg, 0.54 mmol), ethyl 4-bromo-1-(2-trimethylsilylethoxymethyl) imidazole-2-carboxylate (224.6 mg, 0.64 mmol), K₃PO₄ (227.5 mg, 1.07 mmol) and ditert-butyl(cyclopentyl)phosphane;dichloropalladium;iron (34.93 mg, 54 μmol) in THF (2 mL) and H₂O) (0.5 mL) was degassed and purged with N₂×3. Then it was heated at 80° C. for 12 h under N₂. After completion, the reaction was filtered through a celite pad. The filtrate was concentrated and purified by prep-TLC (25% EtOAc in PE) to afford the title compound (62 mg, 22.4%) as a brown oil. MS (ESI): mass calcd, for C₂₃H₃₂F₃N₃O₅Si: 515.21, found: 516.2 [M+H]⁺.

### 4-(4-(3-(Ethylamino)-1,1,1-trifluoro-2-hydroxy-3-oxopropan-2-yl)phenyl)-1-((2-(trimethylsilyl)ethoxy)methyl)-1H-imidazole-2-carboxylic Acid

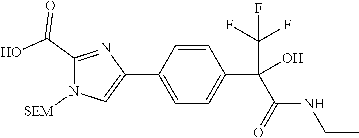 [SMILES: CCNC(=O)C(O)(c1ccc(-c2cn(COCC[Si](C)(C)C)c(C(=O)O)n2)cc1)C(F)(F)F]

To a solution of ethyl 4-(4-(3-(ethylamino)-1,1,1-trifluoro-2-hydroxy-3-oxopropan-2-yl)phenyl)-1-((2-(trimethylsilyl)ethoxy)methyl)-1H-imidazole-2-carboxylate (0.06 g, 0.12 mmol) in THF (0.5 ml) and H₂O (0.1 mL) was added LiOH·H₂O (14.7 mg, 0.35 mmol). The resulting suspension was stirred at 20° C. for 12 h. After completion, the reaction mixture was adjusted to pH=3-4 by 1 N HCl and extracted with EtOAc (5 mL×3). The combined organic layer was washed with brine, dried over anhydrous Na₂SO₄, filtered and concentrated to afford the crude title compound (50 mg, 88.3%) as a brown solid, which was directly used in the next step without further purification. MS (ESI): mass calcd, for C₂₁H₂₈F₃N₃O₅Si: 487.18, found: 488.2 [M+H]⁺.

### 4-(4-(3-(Ethylamino)-1,1,1-trifluoro-2-hydroxy-3-oxopropan-2-yl)phenyl)-N-phenyl-1-((2-(trimethylsilyl)ethoxy)methyl)-1H-imidazole-2-carboxamide

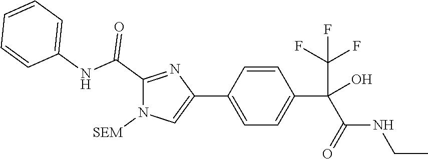 [SMILES: CCNC(=O)C(O)(c1ccc(-c2cn(COCC[Si](C)(C)C)c(C(=O)Nc3ccccc3)n2)cc1)C(F)(F)F]

To an ice-cold solution of 4-(4-(3-(ethylamino)-1,1,1-trifluoro-2-hydroxy-3-oxopropan-2-yl)phenyl)-1-((2-(trimethylsilyl)ethoxy)methyl)-1H-imidazole-2-carboxylic acid (0.05 g, 0.1 mmol) and DIPEA (54 μL, 0.31 mmol) in DMF (1 mL) was added HATU (78 mg, 0.21 mmol). Then it was stirred at 20° C. for 15 minutes, followed by addition of aniline (11.2 μL, 0.12 mmol). The reaction was allowed to stir at rt for an additional 2 h. After completion, the reaction was poured into H₂O (5 mL) and extracted with EtOAc (5 mL×3). The combined organic layer was washed with brine, dried over anhydrous Na₂SO₄, filtered and concentrated. The crude was purified by prop-TLC (50% EtOAc in PE) to afford the title compound (12 mg, 20.8%) as a white solid. MS (ESI): mass calcd, for C₂₇H₃₃F₃N₄O₄Si: 562.22, found: 563.3 [M+H]⁺.

### 4-(4-(3-(Ethylamino)-1,1,1-trifluoro-2-hydroxy-3-oxopropan-2-yl)phenyl)-N-phenyl-1H-imidazole-2-carboxamide (467)

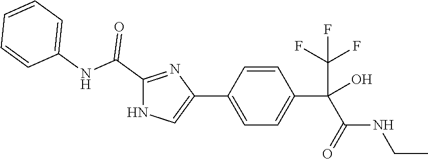 [SMILES: CCNC(=O)C(O)(c1ccc(-c2c[nH]c(C(=O)Nc3ccccc3)n2)cc1)C(F)(F)F]

4-(4-(3-(Ethylamino)-1,1,1-trifluoro-2-hydroxy-3-oxopropan-2-yl)phenyl)-N-phenyl-1-((2-(trimethylsilyl)ethoxy)methyl)-1H-imidazole-2-carboxamide (0.01 g, 0.018 mmol) was dissolved in a solution of TFA (0.5 mL) and H₂O) (0.1 mL). After stirring at rt for 2 h, the reaction was adjusted to pH=7 with sat. Na₂CO₃ and extracted with EtOAc (5 mL×3). The combined organic layer was washed with brine, dried with anhydrous Na₂SO₄, filtered and concentrated. The crude was purified by reverse phase HPLC (gradient elution, 25-55% ACN in H₂O with 10 mM NH₄HCO₃ as a modifier) to afford the title compound (467) (3.8 mg, 49.5%, racemic) as a white solid. MS (ESI): mass calcd, for C₂₁H₁₉F₃N₄O₃: 432.14, found: 433.1 [M+H]⁺. ¹H NMR (400 MHz, DMSO-d6) δ ppm 13.38 (s, 1H), 10.23 (s, 1H), 8.24 (t, J=5.6 Hz, 1H), 7.94 (d, J=8.4 Hz, 2H), 7.86 (d, J=8.0 Hz, 3H), 7.68 (d, J=8.4 Hz, 3H), 7.36 (t, J=8.0 Hz, 2H), 7.11 (t, J=6.0 Hz, 1H), 3.11-3.15 (m, 2H), 1.00 (t, J=7.2 Hz, 3H).

Examples 469, 470, 471, 472, 474, 475, 477, 479, 480, 481, 482, 483, 484, 486, and 487 were synthesized in similar procedures as described in Example 467.

The following compounds were synthesized in a similar procedure as described in the second to last step of preparing Example 467 (using HATU) from their respective carboxylic acid and amine.

Example 307 from (S)-2-(2-aminobenzo[d]thiazol-6-yl)-N-ethyl-3,3,3-trifluoro-2-hydroxypropanamide and 1H-indole-2-carboxylic acid, Example 308 from (S)-2-(2-aminobenzo[d]thiazol-6-yl)-N-ethyl-3,3,3-trifluoro-2-hydroxypropanamide and 7-methyl-1H-indole-2-carboxylic acid, Example 354 (heated to 50° C.) from (S)-2-(2-aminobenzo[d]thiazol-6-yl)-N-ethyl-3,3,3-trifluoro-2-hydroxypropanamide and 5-fluoro-1H-indole-2-carboxylic acid, Example 364 from (S)-3,3,3-trifluoro-2-(2-(3-(4-fluorophenyl)ureido)benzo[d]thiazol-6-yl)-2-hydroxypropanoic acid and 3-fluoropropan-1-amine, Example 365, Example 373, Example 374 were synthesized from (S)-3,3,3-trifluoro-2-(2-(3-(4-fluorophenyl)ureido)benzo[d]thiazol-6-yl)-2-hydroxypropanoic acid and the HCl salt of 3-(pyrimidin-2-yl)cyclobutan-1-amine, and Example 382 from (S)-3,3,3-trifluoro-2-(2-(3-(4-fluorophenyl)ureido)benzo[d]thiazol-6-yl)-2-hydroxypropanoic acid and the HCl salt of (1s,3s)-3-(methylsulfonyl)cyclobutan-1-amine,

Example 363 was synthesized from (S)-2-(2-aminobenzo[d]thiazol-6-yl)-N-ethyl-3,3,3-trifluoro-2-hydroxypropanamide and 5-methyl-1H-pyrrolo[2,3-b]pyridine-2-carboxylic acid in a similar procedure as described in the second to last step of preparing Example 467, except BOP ((benzotriazolyloxy)tris(dimetylamine)phosphonium hexafluophosphate) was used instead of HATU.

Examples 371 and Example 372 were synthesized in a similar procedure from (S)-2-(2-aminobenzo[d]thiazol-6-yl)-N-ethyl-3,3,3-trifluoro-2-hydroxypropanamide and their respective carboxylic acids as described in the second to last step of preparing Example 467, except PyAOP was used instead of HATU.

### Example 468. (S)—N-Ethyl-3,3,3-trifluoro-2-hydroxy-2-(2-((4-oxo-4,5,6,7-tetrahydro-1H-cyclopenta[d]pyrimidin-2-yl)amino)benzo[d]thiazol-6-yl)propanamide (468)

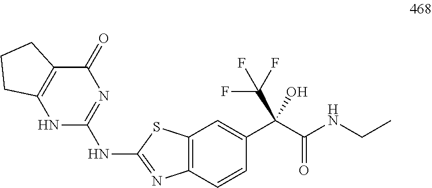 [SMILES: CCNC(=O)[C@@](O)(c1ccc2nc(Nc3nc(=O)c4c([nH]3)CCC4)sc2c1)C(F)(F)F]

### Synthetic Scheme:

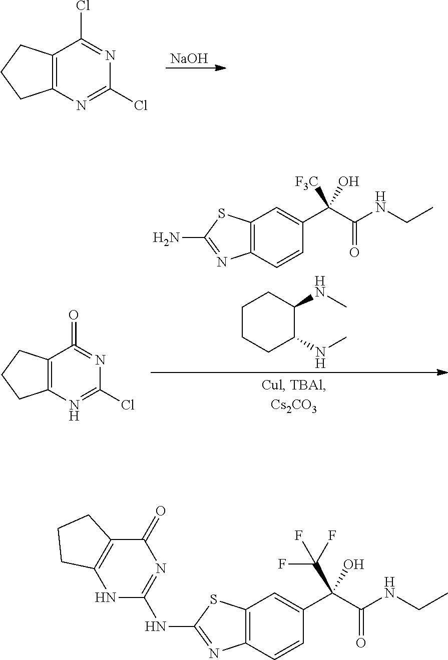

### 2-Chloro-1,5,6,7-tetrahydro-4H-cyclopenta[d]pyrimidin-4-one

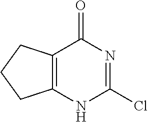 [SMILES: O=c1nc(Cl)[nH]c2c1CCC2]

To a solution of 2,4-dichloro-6,7-dihydro-5H-cyclopenta[d]pyrimidine (0.8 g, 4.23 mmol) in THF (4 mL) was added 5 N NaOH (4.00 mL, 20 mmol). Then it was stirred at 80° C. After 12 h, the reaction mixture was diluted with water and washed twice with DCM. The aqueous layer was acidified to pH=S by 10% KHSO₄ and further extracted with DCM (15 mL×3). The combined organic layer was dried over Na₂SO₄, filtered, concentrated, and purified by prep-TLC (50% EtOAc in PE) to afford the title compound (0.014 g, 7.8%) as a white solid. MS (ESI): mass calcd, for C₇H₇ClN₂O: 170.02, found: 170.9 [M+H]⁺.

### (S)—N-Ethyl-3,3,3-trifluoro-2-hydroxy-2-(2-((4-oxo-4,5,6,7-tetrahydro-1H-cyclopenta[d]pyrimidin-2-yl)amino)benzo[d]thiazol-6-yl)propenamide (468)

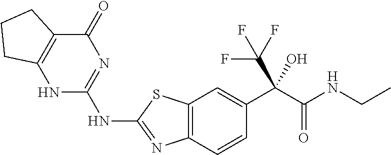 [SMILES: CCNC(=O)[C@@](O)(c1ccc2nc(Nc3nc(=O)c4c([nH]3)CCC4)sc2c1)C(F)(F)F]

To a suspension of (2S)-2-(2-amino-1,3-benzothiazol-6-yl)-N-ethyl-3,3,3-trifluoro-2-hydroxy-propanamide (93.6 mg, 0.29 mmol), Cs₂CO₃ (191 mg, 0.59 mmol), (1R,2R)—N1,N2-dimethylcyclohexane-1,2-diamine (16.7 mg, 0.12 mmol) and 2-chloro-1,5,6,7-tetrahydro-4H-cyclopenta[d]pyrimidin-4-one (0.05 g, 0.29 mmol) in DMA (1 mL) was added CuI·TBAl (65.6 mg, 0.059 mmol) under N₂. Then it was stirred at 120° C. for 12 h. Upon completion, the reaction was diluted with H₂O (5 mL) and extracted with EtOAc (3 mL×5). The combined organic layer was washed with brine, dried over Na₂SO₄, filtered and concentrated. The crude was purified by reverse phase HPLC (gradient elution, 10-45% ACN in H₂O with 0.2% FA as a modifier) to afford the title compound (468) (0.017 g, 12.8%) as a white solid. MS (ESI): mass calcd, for C₁₉H₁₈F₃N₅O₃S: 453.11, found: 454.1 [M+H]⁺. ¹H NMR (400 MHz, DMSO-d6) δ ppm 8.21 (t, J=6.0 Hz, 1H), 8.17 (s, 1H), 7.79 (s, 1H), 7.76 (s, 1H), 7.70 (d, J=8.4 Hz, 1H), 7.49 (d, J=8.8 Hz, 1H), 3.09-3.13 (m, 2H), 2.81 (t, J=7.6 Hz, 2H), 2.66 (t, J=7.2 Hz, 2H), 1.97-2.05 (m, 2H), 0.98 (t, J==7.2 Hz, 3H).

### Example 473. N-Ethyl-3,3,3-trifluoro-2-hydroxy-2-(2-(4-methyl-1H-1,2,3-triazol-1-yl)benzo[d]thiazol-6-yl)propanamide (473)

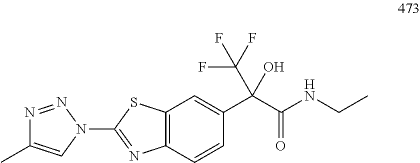 [SMILES: CCNC(=O)C(O)(c1ccc2nc(-n3cc(C)nn3)sc2c1)C(F)(F)F]

### Synthetic Scheme:

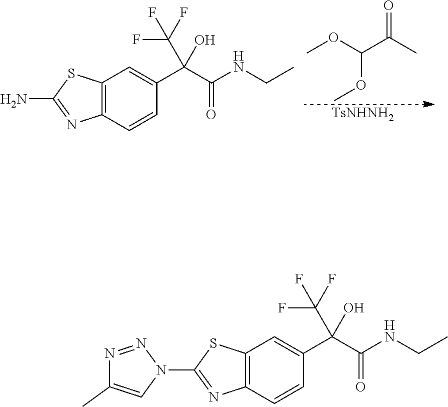

### N-Ethyl-3,3,3-trifluoro-2-hydroxy-2-(2-(4-methyl-1H-1,2,3-triazol-1-yl)benzo[d]thiazol-6-yl)propanamide (473)

A mixture of 2-(2-amino-1,3-benzothiazol-6-yl)-N-ethyl-3,3,3-trifluoro-2-hydroxy-propanamide (0.1 g, 0.31 mmol), 4-methylbenzenesulfonohydrazide (58.3 mg, 0.31 mmol) and 1,1-dimethoxypropan-2-one (37 mg, 0.31 mmol) in DMSO (2 mL) was degassed and purged with N₂×3. Then it was stirred at 80° C. for 12 h under N₂. After completion, the reaction was poured into H₂O (10 mL) and extracted with EtOAc (10 mL×3). The combined organic layer was washed with brine, dried over anhydrous Na₂SO₄, filtered and concentrated. The crude was purified by prep-TLC (50% EtOAc in PE) to afford the title compound with impurities. It was further purified by reverse phase HPLC (gradient elution, 10-45% ACN in H₂O with 10 mM NH₄HCO₃ as a modifier) to afford the title compound (473) (12.1 mg, 10%, racemic) as a white solid. MS (ESI): mass calcd, for C₁₅H₁₄F₃N₅O₂S: 385.08, found: 386.1 [M+H]⁺. ¹H NMR (400 MHz, DMSO-d6) δ ppm 8.11 (s, 1H), 8.50 (d, J=1.2 Hz, 1H), 8.29 (t, J=5.6 Hz, 1H), 8.05 (d, J=8.8 Hz, 1H), 7.99 (s, 1H), 7.86 (d, J=7.6 Hz, 1H), 3.10-3.17 (m, 2H), 2.38 (s, 3H), 1.00 (t, J=7.2 Hz, 3H).

### Example 476. (S)—S-Chloro-N-(6-(3-(ethylamino)-1,1,1-trifluoro-2-hydroxy-3-oxopropan-2-yl)benzo[d]thiazol-2-yl)-4-methyl-1H-imidazole-2-carboxamide (476)

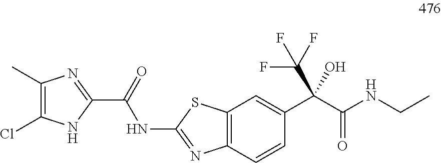 [SMILES: CCNC(=O)[C@@](O)(c1ccc2nc(NC(=O)c3nc(C)c(Cl)[nH]3)sc2c1)C(F)(F)F]

### Synthetic Scheme:

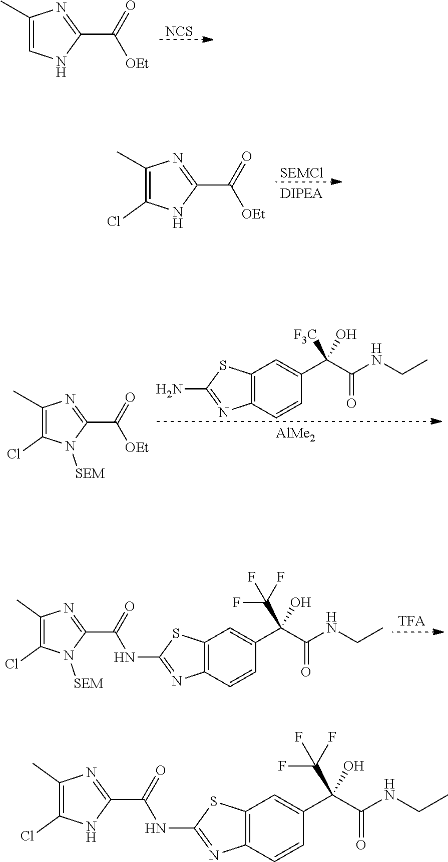

### Ethyl 5-chloro-4-methyl-1H-imidazole-2-carboxylate

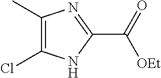

To a solution of ethyl 4-methyl-1H-imidazole-2-carboxylate (0.1 g, 0.65 mmol) in ACN (1 mL) was added NCS (91 mg, 0.68 mmol). After stirring at 25° C. for 12 h, the reaction was quenched with H₂O (2 mL) and extracted with EtOAc (5 mL×2). The combined organic layer was washed with brine, dried over Na₂SO₄, filtered and concentrated. The crude was purified by prep-TLC (50% EtOAc in PE) to provide the title compound (0.07 g, 57.2%) as a white solid. MS (ESI): mass calcd, for C₇H₉ClN₂O₂: 188.04, found: 189.1 [M+H]⁺.

### Ethyl 5-chloro-4-methyl-1-((2-(trimethylsilyl)ethoxy)methyl)-1H-imidazole-2-carboxylate

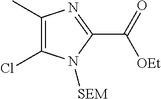 [SMILES: CCOC(=O)c1nc(C)c(Cl)n1COCC[Si](C)(C)C]

To a solution of ethyl 5-chloro-4-methyl-1H-imidazole-2-carboxylate (0.05 g, 0.27 mmol) in DCM (2 mL) was added SEM-Cl (94 μL, 0.53 mmol) and DIPEA (185 μL, 1.06 mmol). After stirring at rt for 3 h, the reaction was quenched with H₂O (2 mL) and extracted with DCM (3 mL×2). The combined organic layer was washed with brine, dried over Na₂SO₄, filtered and concentrated. The crude was purified by prep-TLC (17% EtOAc in PE) to afford the title compound (0.05 g, 59%) as a white solid. MS (ESI): mass calcd, for C₁₃H₂₃ClN₂O₃Si: 318.12, found: 319.1 [M+H]⁺.

### (S)—S-Chloro-N-(6-(3-(ethylamino)-1,1,1-trifluoro-2-hydroxy-3-oxopropan-2-yl)benzo[d]thiazol-2-yl)-4-methyl-1-((2-(trimethylsilyl)ethoxy)methyl)-1H-imidazole-2-carboxamide

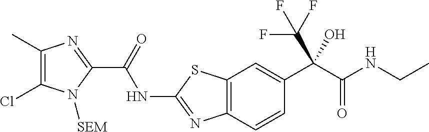 [SMILES: CCNC(=O)[C@@](O)(c1ccc2nc(NC(=O)c3nc(C)c(Cl)n3COCC[Si](C)(C)C)sc2c1)C(F)(F)F]

A solution of 2 M AlMe₃ in toluene (0.78 mL, 1.57 mmol) was added dropwise to an ice-cold solution of (2S)-2-(2-amino-1,3-benzothiazol-6-yl)-N-ethyl-3,3,3-trifluoro-2-hydroxy-propanamide (250 mg, 0.78 mmol) in toluene (1 mL) under N₂. Then it was stirred at 40° C. for 40 minutes, followed by dropwise addition of ethyl 5-chloro-4-methyl-1-((2-(trimethylsilyl)ethoxy)methyl)-1H-imidazole-2-carboxylate (0.05 g, 0.16 mmol). The resulting solution was allowed to heat at 80° C. for 12 h and cooled to rt. The reaction was quenched with sat. NH₄Cl (10 mL) and extracted with EtOAc (20 mL×3). The combined organic layer was washed with brine, dried over Na₂SO₄, filtered and concentrated. The crude was purified by prep-TLC (50% EtOAc in PE) to afford the title compound (0.02 g, 21.5%) as a yellow solid. MS (ESI): mass calcd, for C₂₃H₂₉ClF₃N₅O₄SSi: 591.14, found: 592.2 [M+H]⁺.

### (S)-5-Chloro-N-(6-(3-(ethylamino)-1,1,1-trifluoro-2-hydroxy-3-oxopropan-2-yl)benzo[d]thiazol-2-yl)-4-methyl-1H-imidazole-2-carboxamide (476)

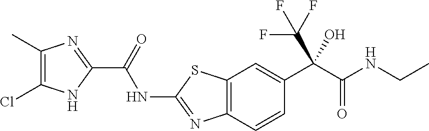 [SMILES: CCNC(=O)[C@@](O)(c1ccc2nc(NC(=O)c3nc(C)c(Cl)[nH]3)sc2c1)C(F)(F)F]

(S)—S-Chloro-N-(6-(3-(ethylamino)-1,1,1-trifluoro-2-hydroxy-3-oxopropan-2-yl)benzo[d]thiazol-2-yl)-4-methyl-1-((2-(trimethylsilyl)ethoxy)methyl)-1H-imidazole-2-carboxamide (0.02 g, 34 μmol) was dissolved in a solution of water (0.1 mL) and TFA (0.5 mL) at 25° C. After stirring at rt for 1 h, the reaction was quenched with sat. NaHCO₃ and extracted with EtOAc (1 mL×3). The combined organic layer was washed with brine, dried over anhydrous Na₂SO₄, filtered, and concentrated. The crude was purified by reverse phase HPLC (gradient elution, 20-50% ACN in H₂O with 10 mM NH₄HCO₃ as a modifier) to afford the title compound (476) (2.1 mg, 13.5%) as a white solid. MS (ESI): mass calcd. for C₁₇H₁₅ClF₃N₅O₃S: 561.05, found: 462.0 [M+H]⁺. ¹H NMR (400 MHz, DMSO-d₆) δ ppm 13.81 (s, 1H), 12.46 (s, 1H), 8.22-8.24 (m, 2H), 7.73-7.77 (m, 1H), 7.70-7.72 (m, 1H), 7.68-7.69 (m, 1H). 3.11-3.15 (m, 2H), 2.23 (s, 3H), 1.00 (t, J=7.2 Hz, 3H).

Examples 478, 488 were synthesized in similar procedures as described in Example 476.

### Example 485. (S)—N-(6-(3-(Ethylamino)-1,1,1-trifluoro-2-hydroxy-3-oxopropan-2-yl)benzo[d]thiazol-2-yl)-5-(trifluoromethyl)-1H-imidazole-2-carboxamide (485)

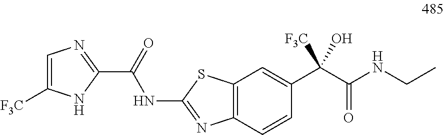 [SMILES: CCNC(=O)[C@@](O)(c1ccc2nc(NC(=O)c3ncc(C(F)(F)F)[nH]3)sc2c1)C(F)(F)F]

### Synthetic Scheme:

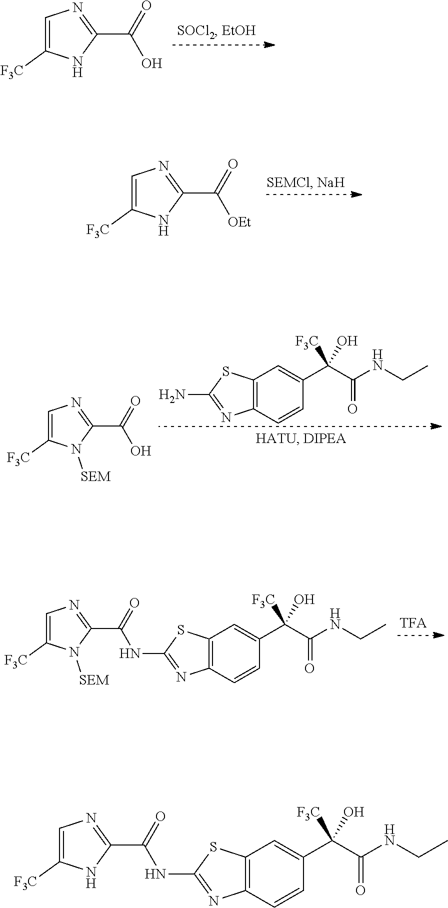 [SMILES: O=C(O)c1ncc(C(F)(F)F)[nH]1 ; CCOC(=O)c1ncc(C(F)(F)F)[nH]1 ; C[Si](C)(C)CCOCn1c(C(F)(F)F)cnc1C(=O)O ; CCNC(=O)[C@@](O)(c1ccc2nc(N)sc2c1)C(F)(F)F ; CCNC(=O)[C@@](O)(c1ccc2nc(NC(=O)c3ncc(C(F)(F)F)n3COCC[Si](C)(C)C)sc2c1)C(F)(F)F ; CCNC(=O)[C@@](O)(c1ccc2nc(NC(=O)c3ncc(C(F)(F)F)[nH]3)sc2c1)C(F)(F)F]

### Ethyl 5-(trifluoromethyl)-1H-imidazole-2-carboxylate

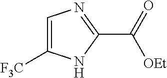 [SMILES: CCOC(=O)c1ncc(C(F)(F)F)[nH]1]

To an ice-cold solution of 5-(trifluoromethyl)-1H-imidazole-2-carboxylic acid (0.7 g, 3.9 mmol) in EtOH (16 mL) was added SOCl₂ (1.13 mL, 15.6 mmol) dropwise. After stirring at 60° C. for 12 h, the reaction solvent was removed under reduced pressure. The resulting residue was poured into H₂O (10 mL) and extracted with EtOAc (10 mL×3). The combined organic layer was washed with brine, dried over anhydrous Na₂SO₄, filtered, and concentrated to afford the title compound (0.7 g, 86.5%) as a white solid, which was used in the next step without further purification. MS (ESI): mass calcd, for C₇H₇F₃N₂O₂: 208.05, found: 209.1 [M+H]⁺.

### S-(Trifluoromethyl)-1-((2-(trimethylsilyl)ethoxy)methyl)-1H-imidazole-2-carboxylic Acid

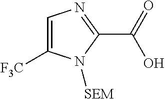 [SMILES: C[Si](C)(C)CCOCn1c(C(F)(F)F)cnc1C(=O)O]

To a solution of ethyl 5-(trifluoromethyl)-1H-imidazole-2-carboxylate (0.5 g, 2.4 mmol) in THE (10 mL) was added SEMCl (0.51 mL, 2.88 mmol) and 60% NaH (192 mg, 4.8 mmol). After stirring at 25° C. for 12 h. the reaction was quenched with sat. NH₄Cl (20 mL) and extracted with DCM (20 mL×3). The combined organic layer was washed with brine, dried over anhydrous Na₂SO₄, filtered and concentrated. The crude was purified by silica gel column chromatography (gradient elution, 0-100% EtOAc in PE) to provide the title compound (0.45 g, 60%) as a white solid. MS (ESI): mass calcd, for C₁₁H₁₇F₃N₂O₃Si: 310.10, found: 311.2 [M+H]⁺.

### (S)—N-(6-(3-(Ethylamino)-1,1,1-trifluoro-2-hydroxy-3-oxopropan-2-yl)benzo[d]thiazol-2-yl)-5-(trifluoromethyl)-1-((2-(trimethylsilyl)ethoxy)methyl)-1H-imidazole-2-carboxamide

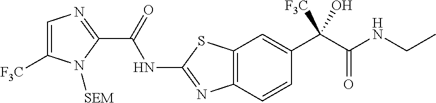 [SMILES: CCNC(=O)[C@@](O)(c1ccc2nc(NC(=O)c3ncc(C(F)(F)F)n3COCC[Si](C)(C)C)sc2c1)C(F)(F)F]

HATU (245 mg, 0.64-mol) was added to an ice-cold solution of 5-(trifluoromethyl)-1-((2-(trimethylsilyl)ethoxy)methyl)-1H-imidazole-2-carboxylic acid (0.1 g, 0.32 mmol) and DIPEA (0.17 mL, 0.97 mmol) in DMF (2 mL). Then it was stirred at 25° C. for 15 minutes, followed by addition of (2S)-2-(2-amino-1,3-benzothiazol-6-yl)-N-ethyl-3,3,3-trifluoro-2-hydroxy-propanamide (described in Example 91, 123.5 mg, 0.39 mmol), After stirring at rt for 4 h, the reaction was poured into H₂O (10 mL) and extracted with EtOAc (10 mL×3). The combined organic layer was washed with brine, dried over anhydrous Na₂SO₄, filtered and concentrated. The crude was purified by prep-TLC (25% EtOAc in PE) to afford the title compound (0.1 g, 50.7%) as a white solid. MS (ESI): mass calcd, for C₂₃H₂₇F₆N₅O₄SSi: 611.15, found: 612.2 [M+H]⁺.

### (S)—N-(6-(3-(Ethylamino)-1,1,1-trifluoro-2-hydroxy-3-oxopropan-2-yl)benzo[d]thiazol-2-yl)-5-(trifluoromethyl)-1H-imidazole-2-carboxamide

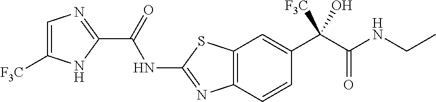 [SMILES: CCNC(=O)[C@@](O)(c1ccc2nc(NC(=O)c3ncc(C(F)(F)F)[nH]3)sc2c1)C(F)(F)F]

(S)—N-(6-(3-(Ethylamino)-1,1,1-trifluoro-2-hydroxy-3-oxopropan-2-yl)benzo[d]thiazol-2-yl)-5-(trifluoromethyl)-1-((2-(trimethylsilyl)ethoxy)methyl)-1H-imidazole-2-carboxamide (0.1 g, 0.16 mmol) was dissolved in a solution of H₂O (1 mL) and TFA (5 mL). After stirring at 25° C. for 12 h, the reaction solution was adjusted to pH=7 with sat. NaHCO₃ and extracted with EtOAc (5 mL×3). The combined organic layer was washed with brine, dried with anhydrous Na₂SO₄, filtered and concentrated. The crude was purified with reverse phase HPLC (gradient elution, 15-45% ACN in H₂O with 10 mM NH₄HCO; as a modifier) to afford the title compound (485) (38.6 mg, 49%) as a white solid. MS (ESI): mass calcd, for C₁₇H₁₃F₆N₅O₃S: 481.06, found: 482.0 [M+H]⁺. ¹H NMR (400 MHz, DMSO-d6) δ ppm 13.85 (s, 1H), 8.28 (s, 1H), 8.24 (t, J=2.0 Hz, 1H), 8.14 (s, 1H), 7.80-7.82 (m, 1H), 3.09-3.17 (m, 2H), 1.00 (t, J=7.2 Hz, 3H).

Examples 390, 405, 415, 421, 430, 431, 432, 448,489, 490, 491 were synthesized in similar procedures as described in Example 485.

The following compounds were synthesized in similar procedures as described in Example 94.

Example 492 (was made staring from 6,7-dihydro-5H-pyrrolo[2,1-c][1,2,4]triazole hydrochloride and used LDA as the base)

Examples 493, 494, 495 were made starting from 2-methyl-1,3,4-oxadiazole.

Examples 497, 498 were obtained through chiral separation of Example 94. Example 500 was made starting from 1-methyl-1H-tetrazole, no SEM protection was needed.

Example 501 was made starting from 2-bromo-5-methyl-1,3,4-thiadiazole, no SEM protection was needed.

Example 502 was made starting from pyrazine, no SEM protection was needed.

Example 503 was made starting from pyridazine, no SEM protection was needed. Example 504 was made starting from 1H-imidazole.

### Example 496. 1-(4-Fluorophenyl)-3-(6-(2,2,2-trifluoro-1-(4-methyl-1H-1,2,3-triazol-1-yl)ethyl)benzo[d]thiazol-2-yl)urea (496)

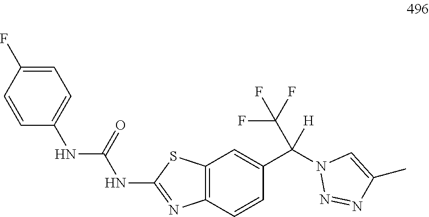 [SMILES: Cc1cn(C(c2ccc3nc(NC(=O)Nc4ccc(F)cc4)sc3c2)C(F)(F)F)nn1]

### Synthetic Scheme:

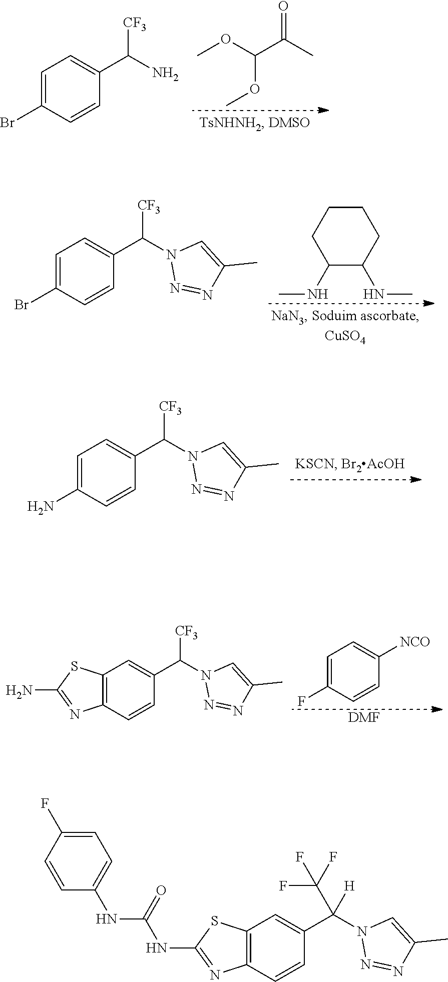

### 1-[1-(4-Bromophenyl)-2,2,2-trifluoro-ethyl]-4-methyl-triazole

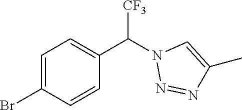 [SMILES: Cc1cn(C(c2ccc(Br)cc2)C(F)(F)F)nn1]

To a solution of 4-methylbenzenesulfonohydrazide (146.6 mg, 0.79 mmol) in DMSO (3 mL) was added 1,1-dimethoxypropan-2-one (93 mg, 0.79 mmol) and 1-(4-bromophenyl)-2,2,2-trifluoro-ethanamine (0.2 g, 0.79 mmol). The reaction was allowed to stir at 80° C. for 12 h under N₂. Upon completion, it was cooled to rt, diluted with H₂O (5 mL) and extracted with EtOAc (5 mL×3). The combined organic layer was washed with brine, dried over Na₂SO₄, filtered and concentrated. The crude was purified by prep-TLC (25% EtOAc in PE) to provide the title compound (0.15 g, 60%) as a yellow oil. MS (ESI): mass calcd. for C₁₁H₉BrF₃N₂: 318.99, found: 320.0 [M+H]⁺.

### 1-(4-Fluorophenyl)-3-(6-(2,2,2-trifluoro-1-(4-methyl-1H-1,2,3-triazol-1-yl)ethyl)benzo[d]thiazol-2-yl)urea (496)

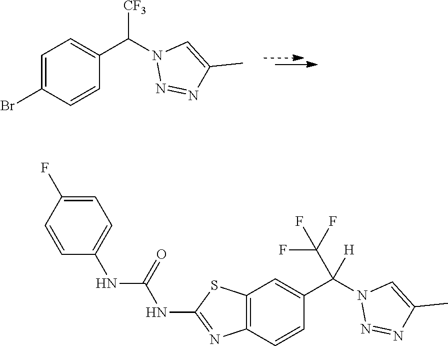 [SMILES: Cc1cn(C(c2ccc(Br)cc2)C(F)(F)F)nn1 ; Cc1cn(C(c2ccc3nc(NC(=O)Nc4ccc(F)cc4)sc3c2)C(F)(F)F)nn1]

The title compound (496) was synthesized from 1-[1-(4-bromophenyl)-2,2,2-trifluoro-ethyl]-4-methyl-triazole in similar procedures as described in Example 1. MS (ESI): mass calcd, for C₁₉H₁₄F₄N₆OS: 450.09, found: 451.1 [M+H]⁺. ¹H NMR (400 MHz, DMSO-d6) δ ppm 11.05 (s, 1H), 9.21 (s, 1H), 8.16-8.25 (m, 2H), 7.62-7.70 (m, 2H), 7.52-7.58 (m, 2H), 7.13-7.20 (m, 3H), 2.26 (s, 3H).

### Example 499. 1-(4-Fluorophenyl)-3-(6-(2,2,2-trifluoro-1-(4-(2-fluoroethyl)-5-methyl-4H-1,2,4-triazol-3-yl)-1-hydroxyethyl)benzo[d]thiazol-2-yl)urea (499)

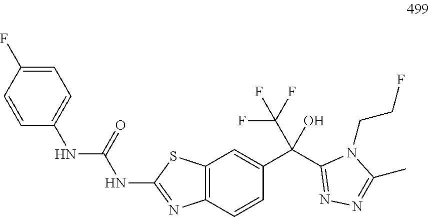 [SMILES: Cc1nnc(C(O)(c2ccc3nc(NC(=O)Nc4ccc(F)cc4)sc3c2)C(F)(F)F)n1CCF]

### Synthetic Scheme:

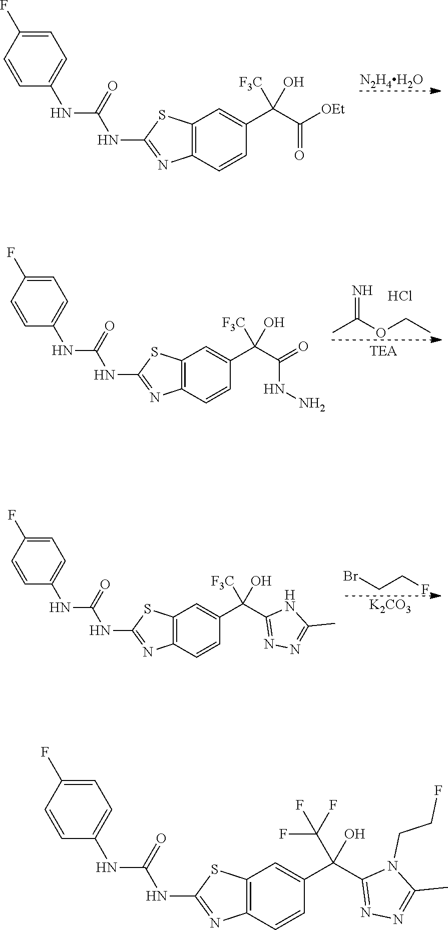

### 1-(4-Fluorophenyl)-3-(6-(1,1,1-trifluoro-3-hydrazineyl-2-hydroxy-3-oxopropan-2-yl)benzo[d]thiazol-2-yl)urea

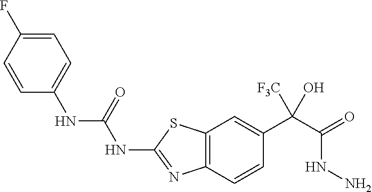 [SMILES: NNC(=O)C(O)(c1ccc2nc(NC(=O)Nc3ccc(F)cc3)sc2c1)C(F)(F)F]

To a solution of ethyl 3,3,3-trifluoro-2-[2-[(4-fluorophenyl) carbamoylamino]-1,3-benzothiazol-6-yl]-2-hydroxy-propanoate (Example 21, 200 mg, 0.44 mmol) in EtOH (2 mL) was added N₂H₄H₂O (106 μL, 2.19 mmol) at 20° C. under N₂. After heating at 90° C. for 12 h, the reaction was diluted with H₂O (5 mL) and extracted with EtOAc (10 mL×3). The combined organic layer was washed with brine, dried over Na₂SO₄, filtered, and concentrated. The crude was purified by prep-TLC (9% MeOH in EtOAc) to afford the title compound (160 mg, 82.5%) as a white solid. MS (ESI): mass calcd, for C₁₇H₁₃F₄N₅O₃S: 443.07, found: 444.1 [M+H]⁺.

### 1-(4-Fluorophenyl)-3-(6-(2,2,2-trifluoro-1-hydroxy-1-(5-methyl-4H-1,2,4-triazol-3-ylethyl)benzo[d]thiazol-2-yl)urea

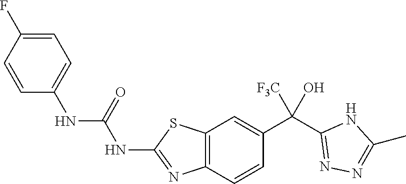 [SMILES: Cc1nnc(C(O)(c2ccc3nc(NC(=O)Nc4ccc(F)cc4)sc3c2)C(F)(F)F)[nH]1]

1-(4-Fluorophenyl)-3-(6-(1,1,1-trifluoro-3-hydrazineyl-2-hydroxy-3-oxopropan-2-yl)benzo[d]thiazol-2-yl)urea (140 mg, 0.32 mmol) was dissolved in a solution of 1-methylpyrrolidin-2-one (1.4 mL), TEA (1.4 mL) in toluene (2.8 mL), followed by addition of ethyl ethanimidate (78 mg, 0.63 mmol). It was then allowed to stir at 100° C. for 12 h. After completion, the reaction was poured into H₂O (10 mL) and extracted with EtOAc (10 mL×3). The combined organic layer was washed with brine, dried over Na₂SO₄, filtered and concentrated. The crude was purified by reverse phase HPLC (gradient elution, 25-55% ACN in H₂O, with 0.2% FA as a modifier) to afford the title compound (14 mg, 9.5%) as a white solid. MS (ESI): mass calcd, for C₁₉H₁₄F₄N₆O₂S: 466.08, found: 467.2 [M+H]⁺.

### 1-(4-Fluorophenyl)-3-(6-(2,2,2-trifluoro-1-(4-(2-fluoroethyl)-5-methyl-4H-1,2,4-triazol-3-yl)-1-hydroxyethyl)benzo[d]thiazol-2-yl)urea

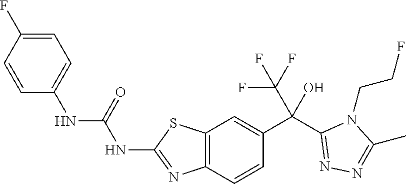 [SMILES: Cc1nnc(C(O)(c2ccc3nc(NC(=O)Nc4ccc(F)cc4)sc3c2)C(F)(F)F)n1CCF]

To a solution of 1-(4-fluorophenyl)-3-(6-(2,2,2-trifluoro-1-hydroxy-1-(5-methyl-4H-1,2,4-triazol-3-yl)ethyl)benzo[d]thiazol-2-yl)urea (14 mg, 30 μmol) in DMF (1 mL) was added 1-bromo-2-fluoro-ethane (4.6 mg, 36 μmol) and K₂CO₃ (8.3 mg, 60 μmol). After heating at 60° C. for 12 h, the reaction was poured into H₂O (10 mL) and extracted with EtOAc (10 mL×3). The combined organic layer was washed with brine, dried over Na₂SO₄, filtered and concentrated. The crude was purified by reverse phase HPLC (gradient elution, 25-55% ACN in H₂O, with 10 mM NH₄HCO₃ as a modifier) to provide the title compound (499) (3 mg, 19.5%, racemic) as a white solid. MS (ESI): mass calcd, for C₂₁H₁₇F₅N₆O₂S: 512.11, found: 513.1 [M+H]⁺. ¹H NMR (400 MHz, DMSO-d6) δ ppm 10.89 (s, 1H), 9.19 (s, 1H), 7.99-8.05 (m, 1H), 7.55 (dd, J=5.2, 9.2 Hz, 3H), 7.45-7.50 (m, 1H), 7.37 (s, 1H), 7.14 (t, J=8.8 Hz, 2H), 4.79-4.84 (m, 1H), 4.68-4.72 (m, 1H), 4.53 (t, J=4.4 Hz, 1H), 4.47 (t, J=4.4 Hz, 1H), 2.43 (s, 3H).

### Example 505. 2-(2,2,2-Trifluoro-1-(2-(3-(4-fluorophenyl)ureido)benzo[d]thiazol-6-yl)-1-hydroxyethyl)-1H-imidazole-5-carboxylic acid (505)

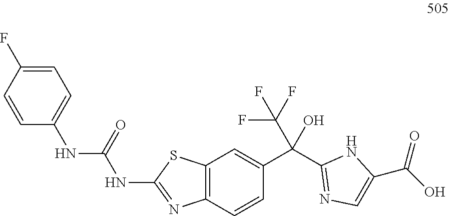 [SMILES: O=C(Nc1ccc(F)cc1)Nc1nc2ccc(C(O)(c3ncc(C(=O)O)[nH]3)C(F)(F)F)cc2s1]

### Synthetic Scheme:

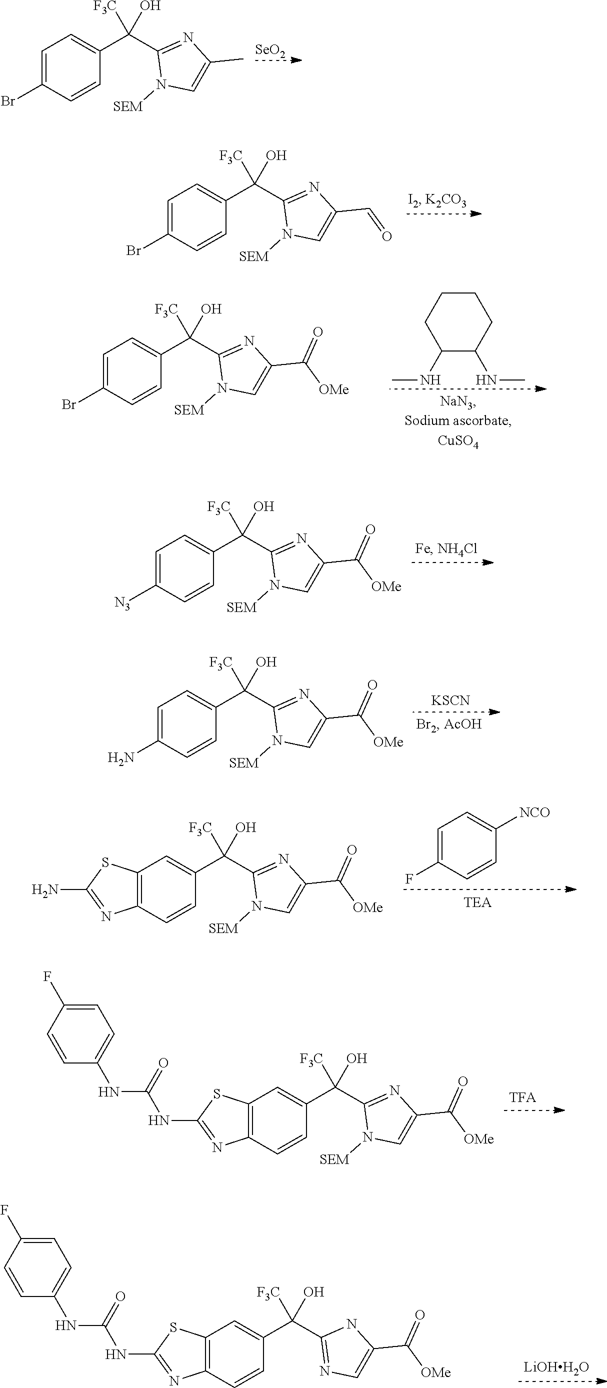

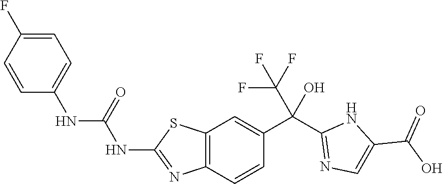 [SMILES: O=C(Nc1ccc(F)cc1)Nc1nc2ccc(C(O)(c3ncc(C(=O)O)[nH]3)C(F)(F)F)cc2s1]

### 2-(1-(4-Bromophenyl)-2,2,2-trifluoro-1-hydroxyethyl)-1-((2-(trimethylsilyl)ethoxy)methyl)-1H-imidazole-4-carbaldehyde

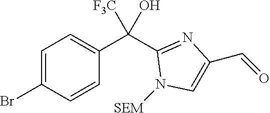 [SMILES: C[Si](C)(C)CCOCn1cc(C=O)nc1C(O)(c1ccc(Br)cc1)C(F)(F)F]

To a solution of 1-(4-bromophenyl)-2,2,2-trifluoro-1-(4-methyl-1-((2-(trimethylsilyl)ethoxy)methyl)-1H-imidazol-2-yl) ethan-1-ol (described in Example 94, 1 g, 2.2 mmol) in dioxane (10 mL) was added SeO₂ (1.17 mL, 10.7 mmol). After heating at 110° C. for 12 h, the reaction was filtered through a celite pad. The filtrate was poured into H₂O (50 mL) and extracted with EtOAc (50 mL×3). The combined organic layer was washed with brine, dried over Na₂SO₄, filtered and concentrated. The crude was purified by silica gel column chromatography (gradient elution, 0-100% EtOAc in PE) to afford the title compound (1.36 g, with impurities) as a white solid. MS (ESI): mass calcd, for C₁₈H₂₂BrF₃N₂O₃Si: 478.05, found: 479.2 [M+H]⁺.

### Methyl 2-(1-(4-bromophenyl)-2,2,2-trifluoro-1-hydroxyethyl)-1-((2-(trimethylsilyl)ethoxy)methyl)-1H-imidazole-4-carboxylate

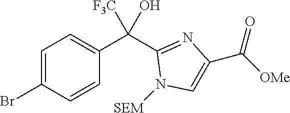 [SMILES: COC(=O)c1cn(COCC[Si](C)(C)C)c(C(O)(c2ccc(Br)cc2)C(F)(F)F)n1]

To a solution of 2-(1-(4-bromophenyl)-2,2,2-trifluoro-1-hydroxyethyl)-1-((2-(trimethylsilyl)ethoxy)methyl)-1H-imidazole-4-carbaldehyde (1.36 g, impure) in MeOH (15 mL) was added I₂ (2.88 g, 11.4 mmol) and K₂CO₃ (2.35 g, 17 mmol). The resulting suspension was stirred at 25° C. for 12 h. After completion, the reaction mixture was poured into H₂O (50 mL) and extracted with EtOAc (50 mL×3). The combined organic layer was washed with brine, dried over Na₂SO₄, filtered and concentrated. The crude was purified by silica gel column chromatography (gradient elution, 0-100% EtOAc in PE) to provide the title compound (0.85 g, 59%) as a white solid. MS (ESI): mass calcd, for C₁₉H₂₄BrF₃N₂O₃Si: 508.06, found: 509.2 [M+H]⁺.

### Methyl 2-(2,2,2-trifluoro-1-(2-(3-(4-fluorophenyl)ureido)benzo[d]thiazol-6-yl)-1-hydroxyethyl)-1-((2-(trimethylsilyl)ethoxy)methyl)-1H-imidazole-4-carboxylate

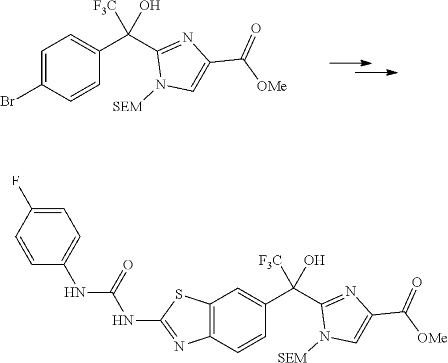 [SMILES: COC(=O)c1cn(COCC[Si](C)(C)C)c(C(O)(c2ccc(Br)cc2)C(F)(F)F)n1 ; COC(=O)c1cn(COCC[Si](C)(C)C)c(C(O)(c2ccc3nc(NC(=O)Nc4ccc(F)cc4)sc3c2)C(F)(F)F)n1]

The title compound was synthesized from methyl 2-(1-(4-bromophenyl)-2,2,2-trifluoro-1-hydroxyethyl)-1-((2-(trimethylsilyl)ethoxy)methyl)-1H-imidazole-4-carboxylate in similar procedures as described in Example 1. MS (ESI): mass calcd, for C₂₇H₂₉F₄N₅O₅SSi: 639.16, found: 640.3 [M+H]⁺.

### Methyl 2-(2,2,2-trifluoro-1-(2-(3-(4-fluorophenyl)ureido)benzo[d]thiazol-6-yl)-1-hydroxyethyl)-1H-imidazole-5-carboxylate

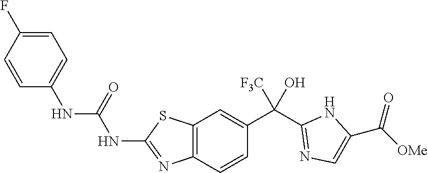 [SMILES: COC(=O)c1cnc(C(O)(c2ccc3nc(NC(=O)Nc4ccc(F)cc4)sc3c2)C(F)(F)F)[nH]1]

Methyl 2-(2,2,2-trifluoro-1-(2-(3-(4-fluorophenyl)ureido)benzo[d]thiazol-6-yl)-1-hydroxyethyl)-1-((2-(trimethylsilyl)ethoxy)methyl)-1H-imidazole-4-carboxylate (60 mg, 0.094 mmol) was dissolved in a solution of TFA (1 mL) and H₂O (0.2 mL). After stirring at 25° C. for 12 h, the reaction was adjusted to pH=7-8 with Na₂CO₃, diluted with H₂O (5 mL) and extracted with EtOAc (10 mL×3). The combined organic layer was dried over Na₂SO₄, filtered and concentrated to afford the title compound (80 mg, with impurities) as a white solid, which was directly used in the next step without further purification. MS (ESI): mass calcd. for C₂₁H₁₅F₄N₅O₄S: 509.08, found: 510.2 [M+H]⁺.

### 2-(2,2,2-Trifluoro-1-(2-(3-(4-fluorophenyl)ureido)benzo[d]thiazol-6-yl)-1-hydroxyethyl)-1H-imidazole-S-carboxylic Acid (505)

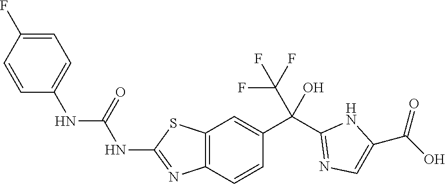 [SMILES: O=C(Nc1ccc(F)cc1)Nc1nc2ccc(C(O)(c3ncc(C(=O)O)[nH]3)C(F)(F)F)cc2s1]

To a solution of methyl 2-(2,2,2-trifluoro-1-(2-(3-(4-fluorophenyl)ureido)benzo[d]thiazol-6-yl)-1-hydroxyethyl)-1H-imidazole-5-carboxylate (30 mg, 0.059 mmol) in THF (1 mL) and H₂O (0.2 mL) was added LiOH·H₂O (12.4 mg, 0.29 mmol). The resulting suspension was stirred at rt for 4 h. After completion, the reaction mixture was diluted with H₂O (20 mL), adjusted to pH=3 by 1 N HCl and extracted with EtOAc (10 mL×3). The combined organic layer was dried over Na₂SO₄, filtered and concentrated. The crude was purified by reverse phase HPLC (gradient elution, 25-55% ACN in H₂O with 0.2% FA as a modifier) to afford the title compound (505) (10 mg, 34%, racemic) as a white solid. MS (ESI): mass calcd, for C₂₀H₁₃F₄N₅O₄S: 495.06, found: 496.1 [M+H]⁺. ¹H NMR (400 MHz, DMSO-d6) δ ppm 12.71 (s, 1H), 12.26 (s, 1H), 10.96 (s, 1H), 9.31 (s, 1H), 8.14 (s, 1H), 7.93 (s, 1H), 7.73 (s, 1H), 7.64 (s, 1H), 7.51-7.56 (m, 3H), 7.18 (t, J=8.8 Hz, 2H).

### Biological Assays

Assays were performed on the example compounds according to assay procedure A or assay procedure B.

### Assay Procedure A

Assays were performed using Expi293F Inducible cells (Invitrogen) stably expressing hPTH1R via a pcZeo TetO DNA plasmid. Cell lines were maintained in suspension in Expi293 Expression Medium (ThermoFisher Scientific) supplemented with 10 μg/mL Blasticidin and 10 μg/mL Zeocin and incubated at 37° C., 8% CO₂, with shaking. To induce receptor expression, hPTH1R cells were incubated in induction medium (Expi293 Expression Medium with 4 μg/mL Doxycycline (Millipore Sigma), 5 mM sodium butyrate (Millipore Sigma) and 100 ng/ml Pertussis toxin (Millipore Sigma)) for 24 hours at 32° C., 5% CO₂, with shaking. Assay-ready aliquots were prepared by harvesting cells 24-hour post-induction. Cells were pelleted at 4° C., resuspended in Expi293 Expression medium+10% DMSO, aliquoted, and kept frozen at −80° C. until ready for use.

For the assay, concentration-response curves of test and reference compounds were added to 384-well plates using an Echo 650 liquid handler (Beckman Coulter) and backfilled with DMSO to a final concentration of 0.3%. CAMP was measured using the cisbio cAMP Gs dynamic HTRF kit (PerkinElmer) according to manufacturer instructions. Aliquots of frozen hPTH1R cells were quickly thawed and washed with phosphate-buffered saline (Sigma-Aldrich) to remove media and DMSO. The cells were resuspended in kit-supplied Stimulation Buffer at 0.2×10⁶ cells/mL. 10 μl of the hPTH1R cell dilution were added to each well of the assay plate and incubated with the compounds for 1 hour in a 37° C., 0% CO₂ incubator. Following this incubation, the cells were lysed and accumulated cAMP was detected through the addition of kit-supplied lysis buffer containing d2-reagent and Eu-cryptate antibody. The HTRF signal was quantified using a BMG Clariostar plate reader optimized for HTRF assays. The HTRF ratio was determined by dividing the signal output at 655 nm by that at 620 nm. Data were normalized to the signal produced by 1 μM PTH (1-34) (100% activation) and vehicle (0% activation).

### Assay Procedure B

Assays were performed using Expi293F Inducible cells (Invitrogen) stably expressing hPTH1R via a pcZeo TetO DNA plasmid. Cell lines were maintained in suspension in Expi293 Expression Medium (ThermoFisher Scientific) supplemented with 10 μg/mL Blasticidin and 10 μg/mL Zeocin and incubated at 37° C., 8% CO2, with shaking. To induce receptor expression, hPTH1R cells were incubated in induction medium (Expi293 Expression Medium with 4 μg/mL Doxycycline (Millipore Sigma), 5 mM sodium butyrate (Millipore Sigma) and 100 ng/mL Pertussis toxin (Millipore Sigma)) for 24 hours at 32° C., 5% CO2, with shaking. Assay-ready aliquots were prepared by harvesting cells 24-hour post-induction. Cells were pelleted at 4° C., resuspended in Expi293 Expression medium+10% DMSO, aliquoted, and kept frozen at −80° C. until ready for use.

For the assay, concentration-response curves of test and reference compounds were added to 384-well plates using an Apricot liquid handler (SPT Labtech) and backfilled with DMSO to a final concentration of 0.3%. cAMP was measured using the cisbio cAMP Gs dynamic HTRF kit (PerkinElmer) according to manufacturer instructions. Aliquots of frozen hPTH1R cells were quickly thawed and washed with phosphate-buffered saline (Sigma-Aldrich) to remove media and DMSO. The cells were resuspended in kit-supplied Stimulation Buffer at 0.2×10⁶ cells/mL. 10 μl of the hPTH1R cell dilution were added to each well of the assay plate and incubated with the compounds for 1 hour in a 37° C., 0% CO2 incubator. Following this incubation, the cells were lysed and accumulated cAMP was detected through the addition of kit-supplied lysis buffer containing d2-reagent and Eu-cryptate antibody. The HTRF signal was quantified using a BMG PHERAstar FSX plate reader optimized for HTRF assays. The HTRF ratio was determined by dividing the signal output at 665 nm by that at 620 nm. Data were normalized to the signal produced by 1 μM PTH(1-34) (100% activation) and vehicle (0% activation). Compounds 87-211 were tested with procedure B.

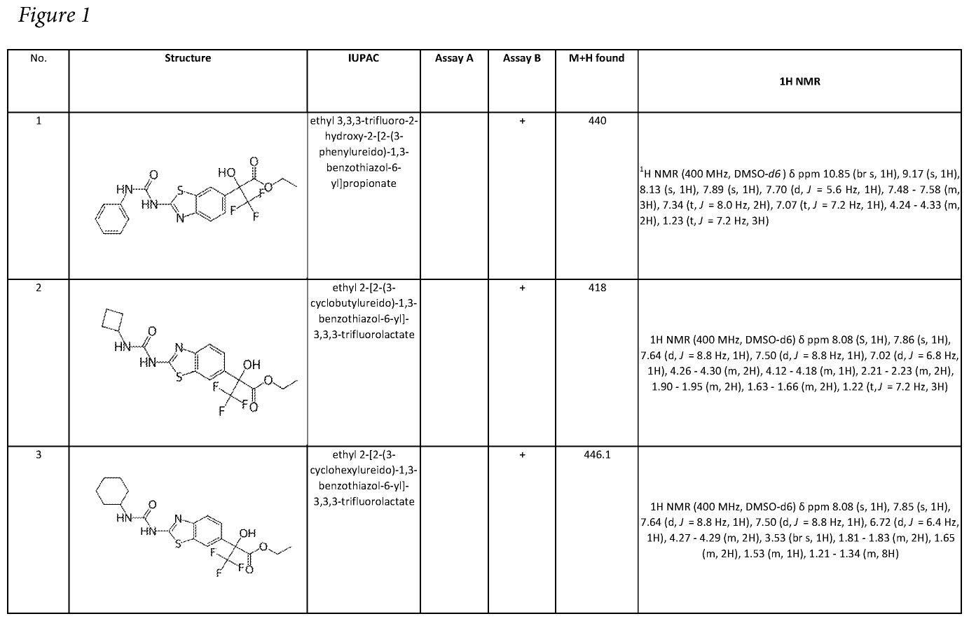

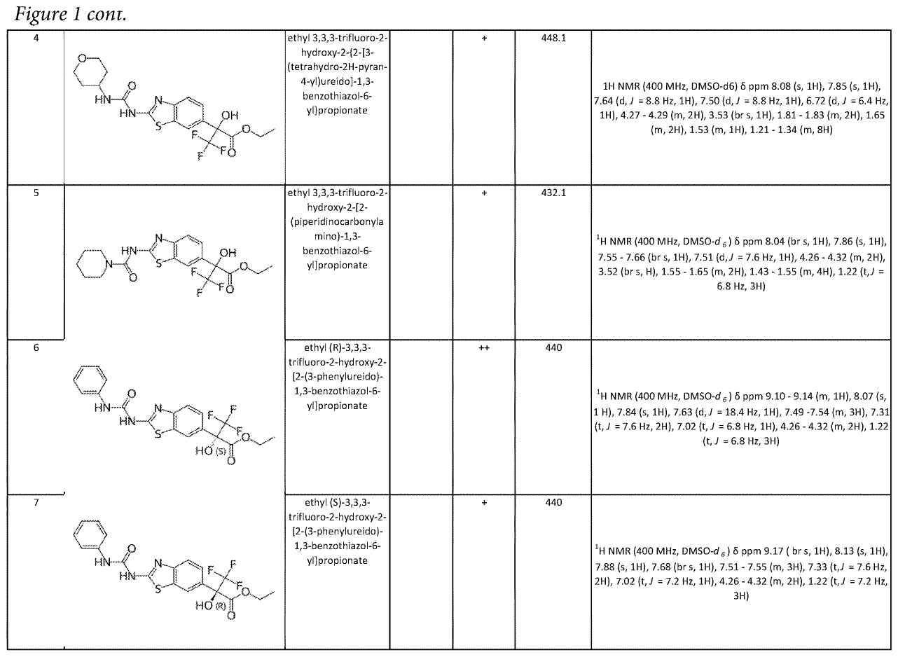

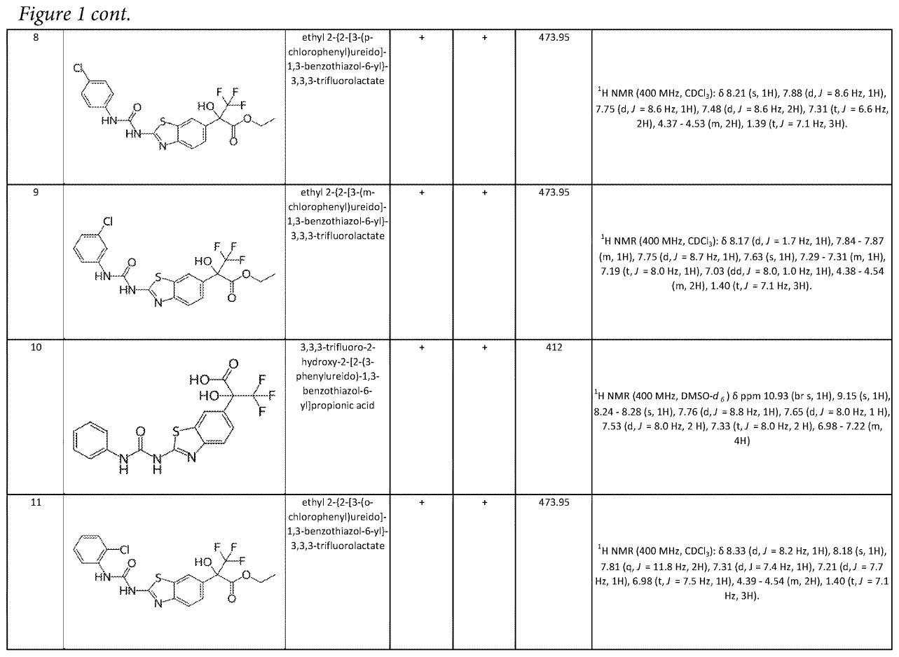

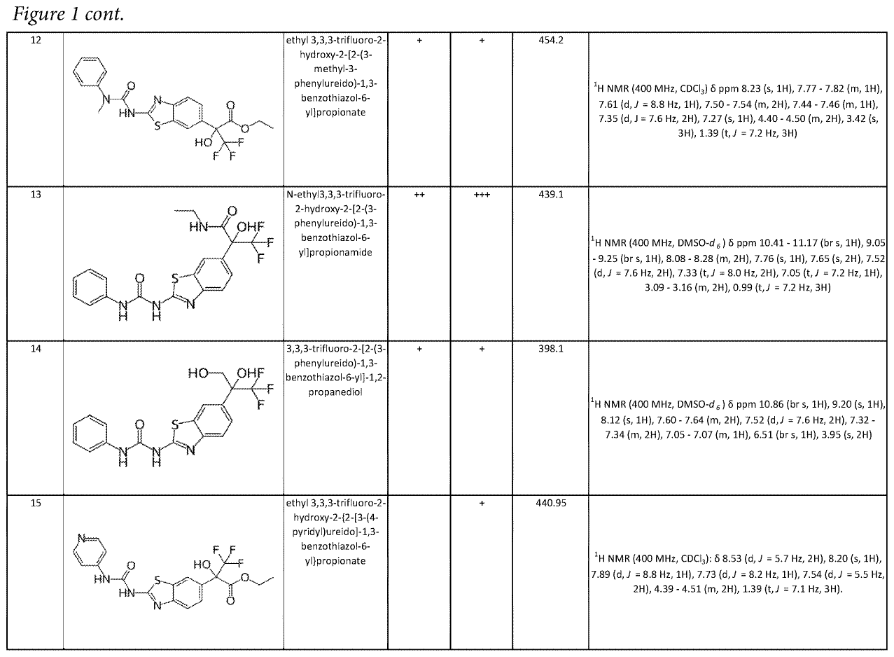

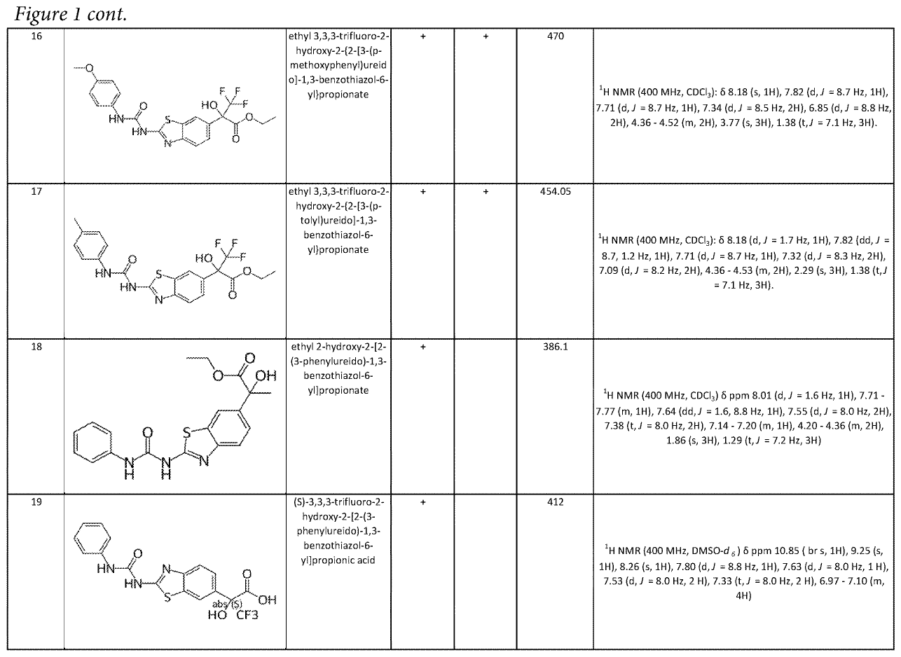

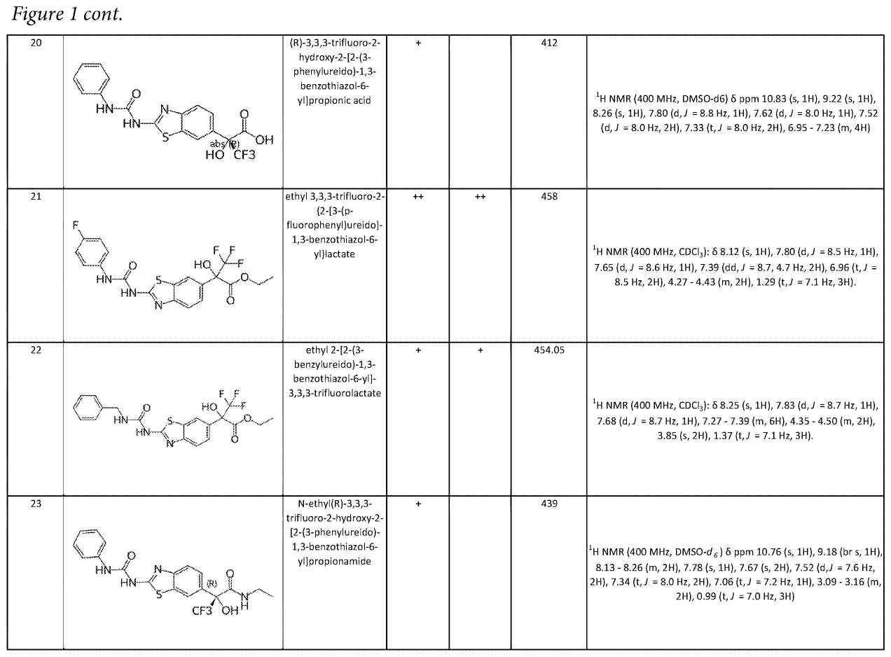

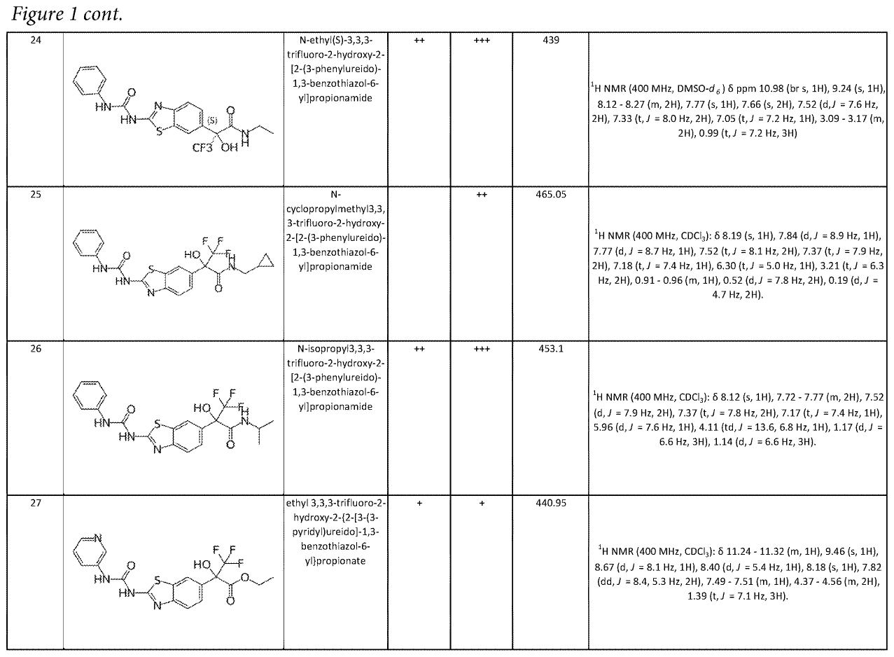

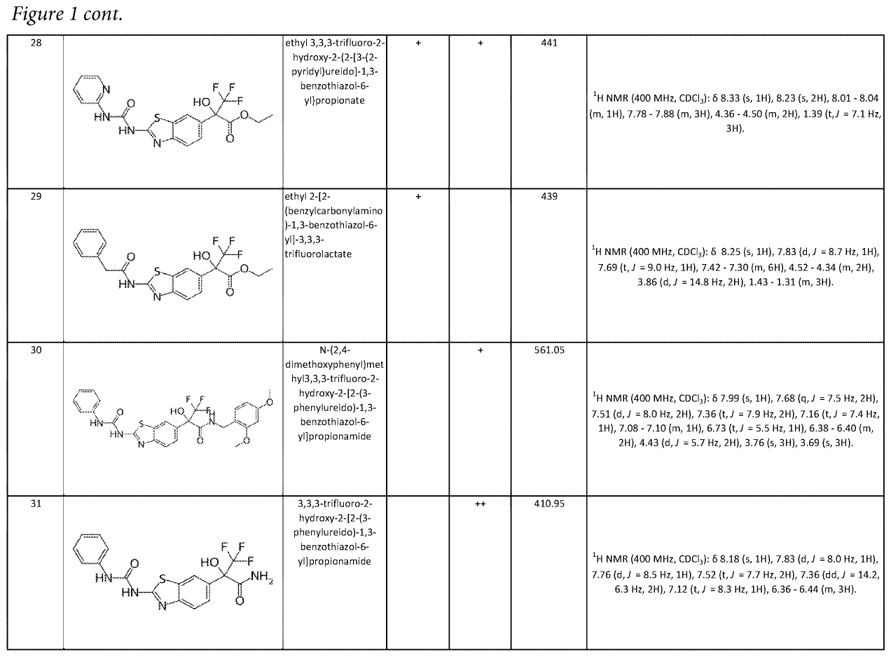

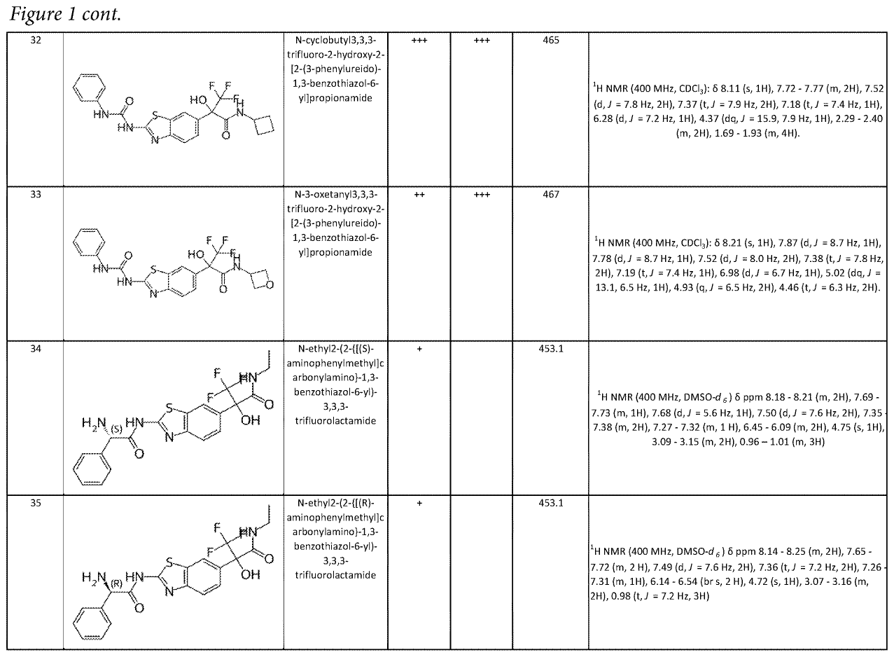

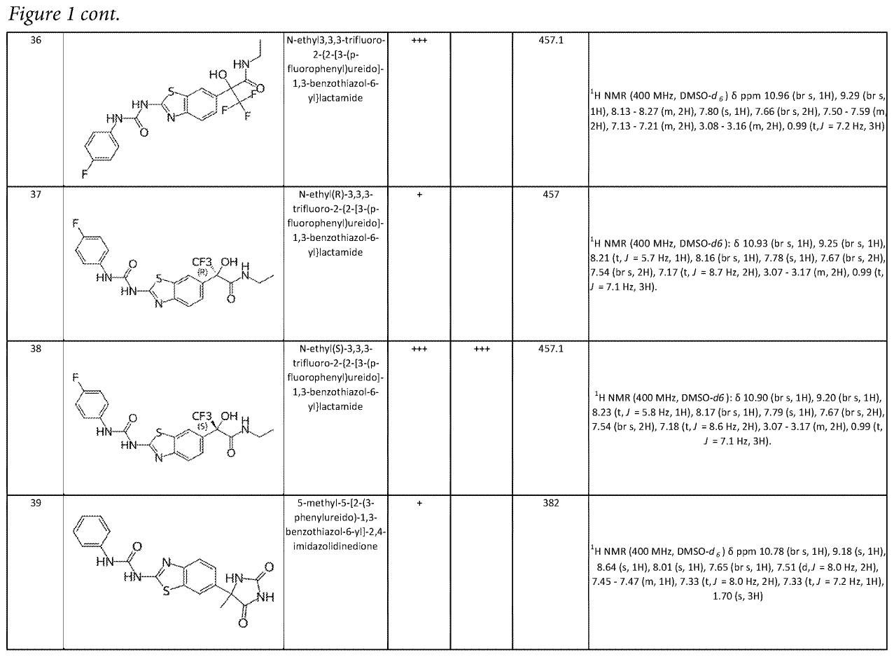

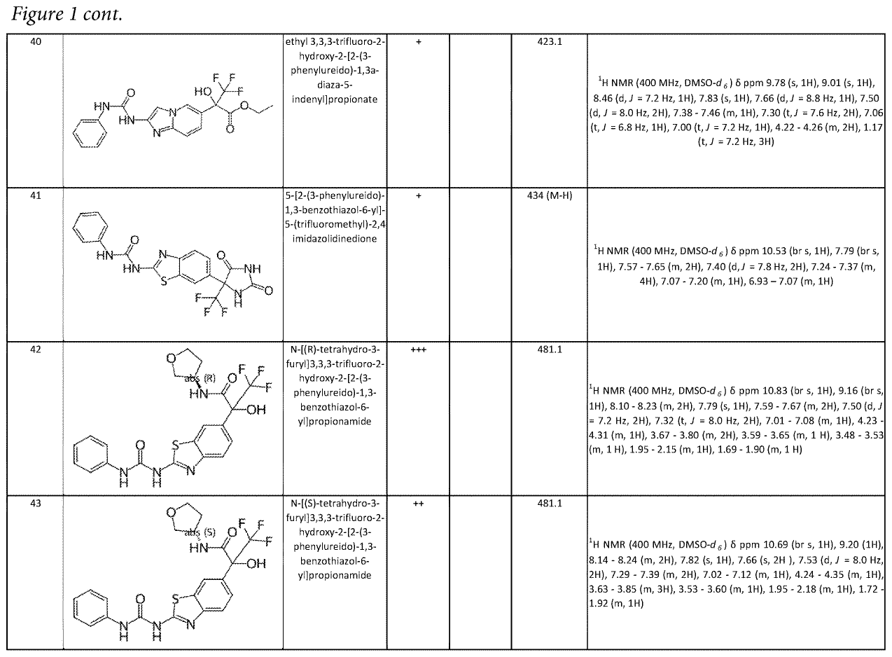

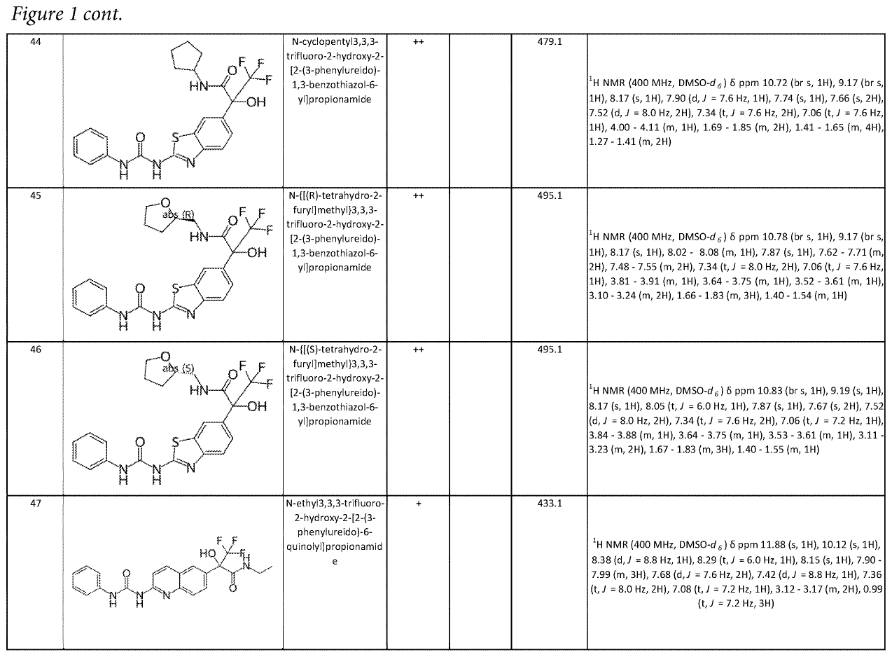

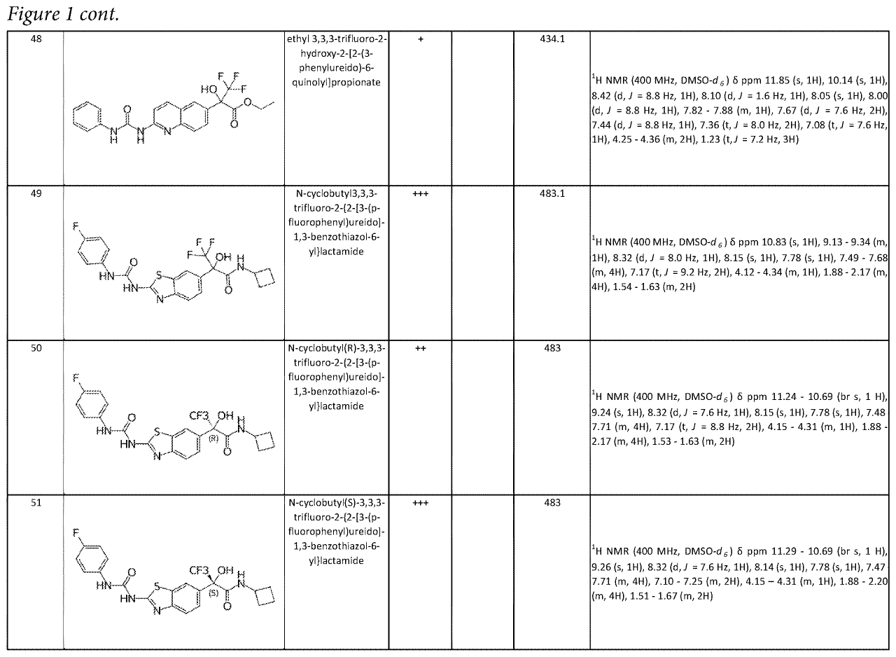

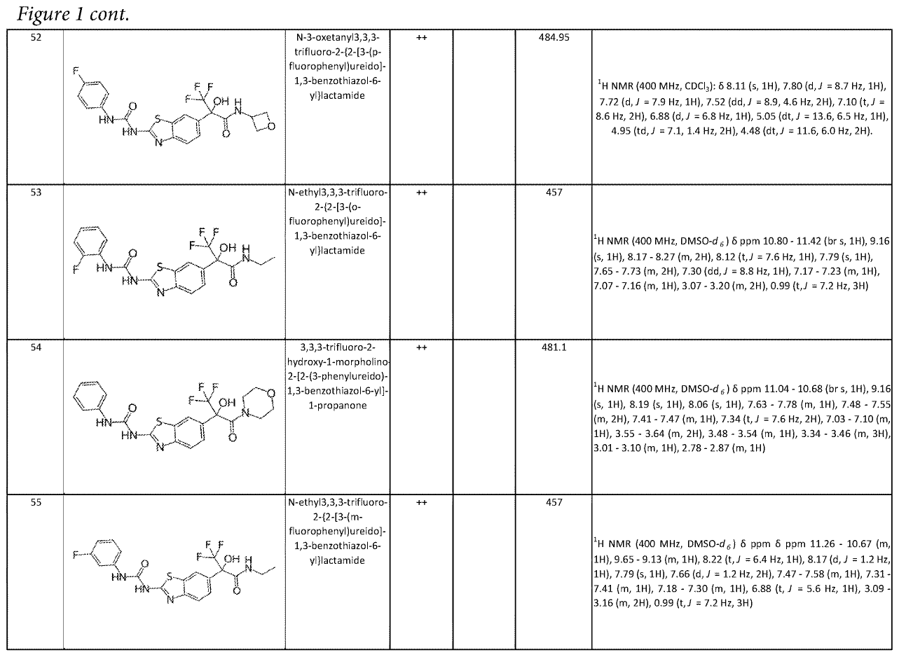

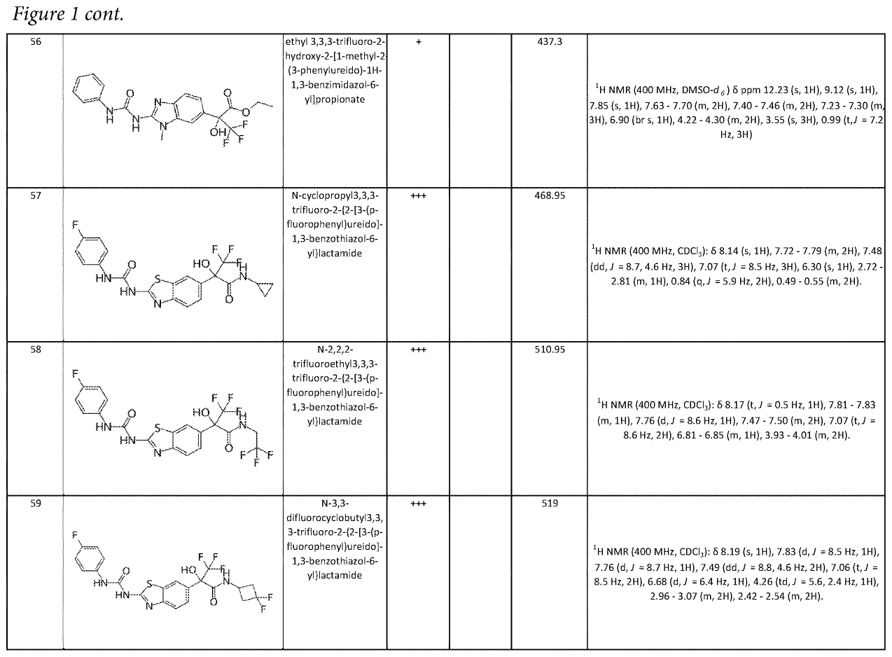

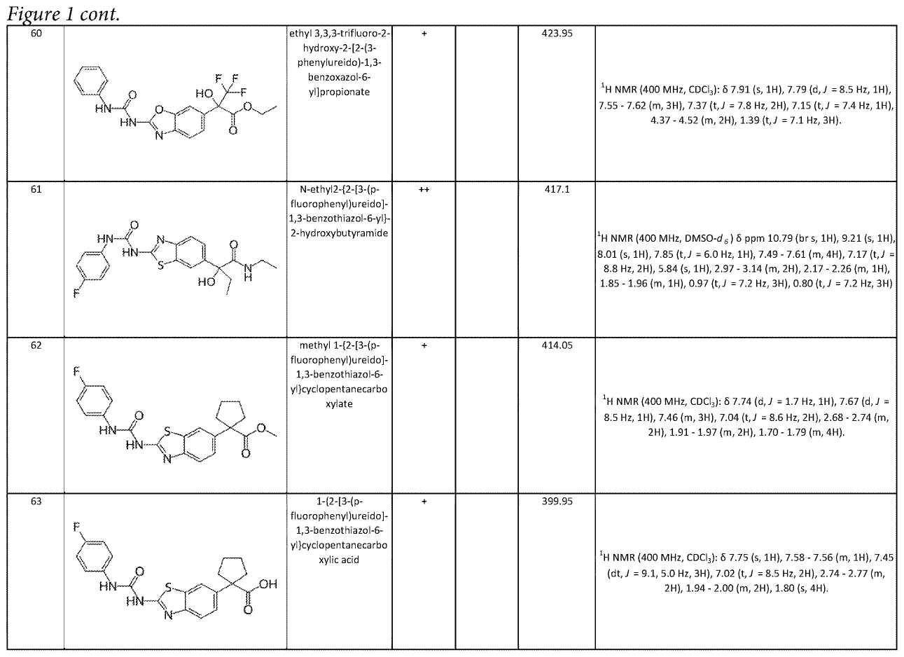

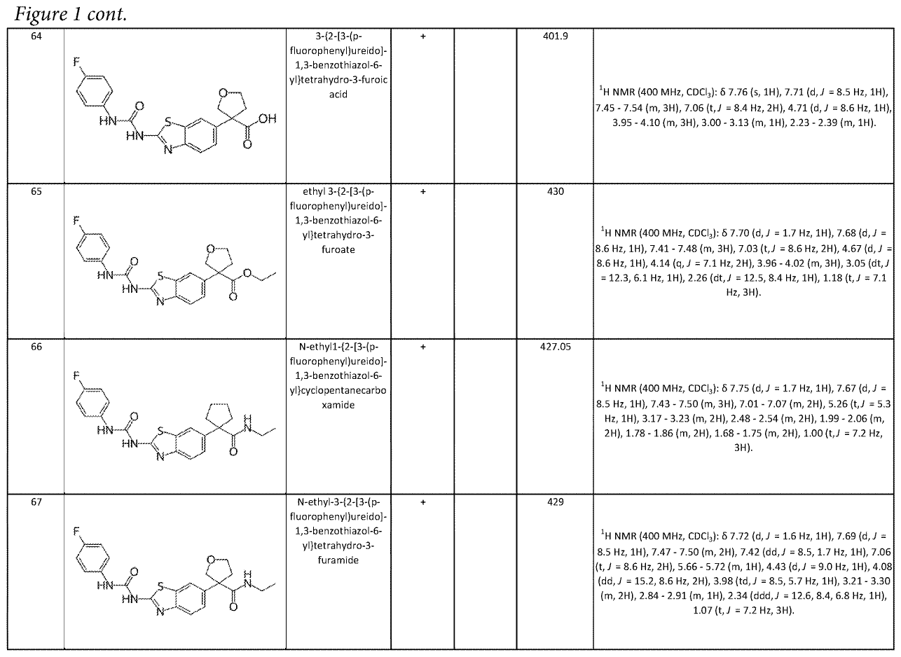

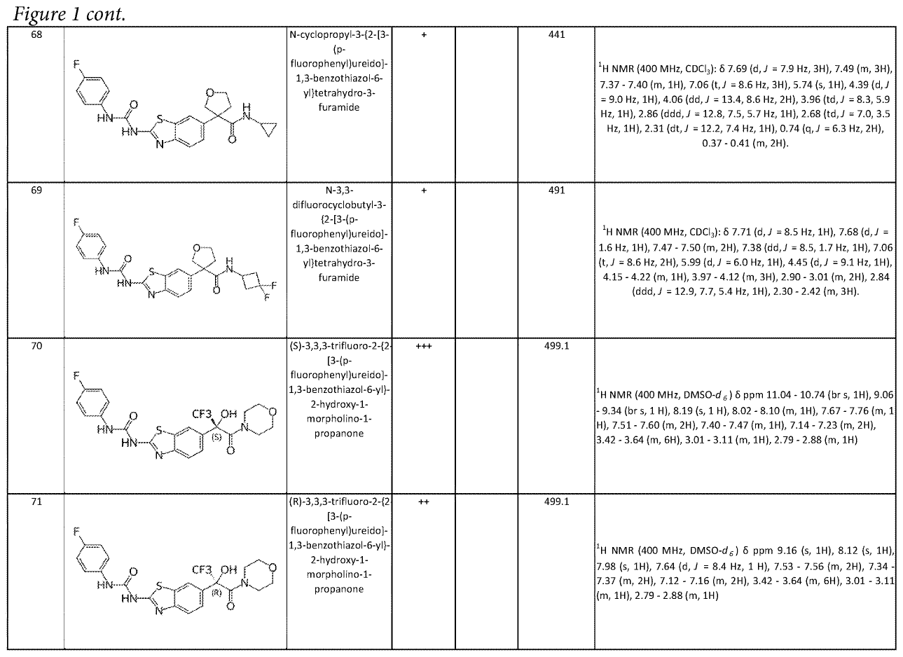

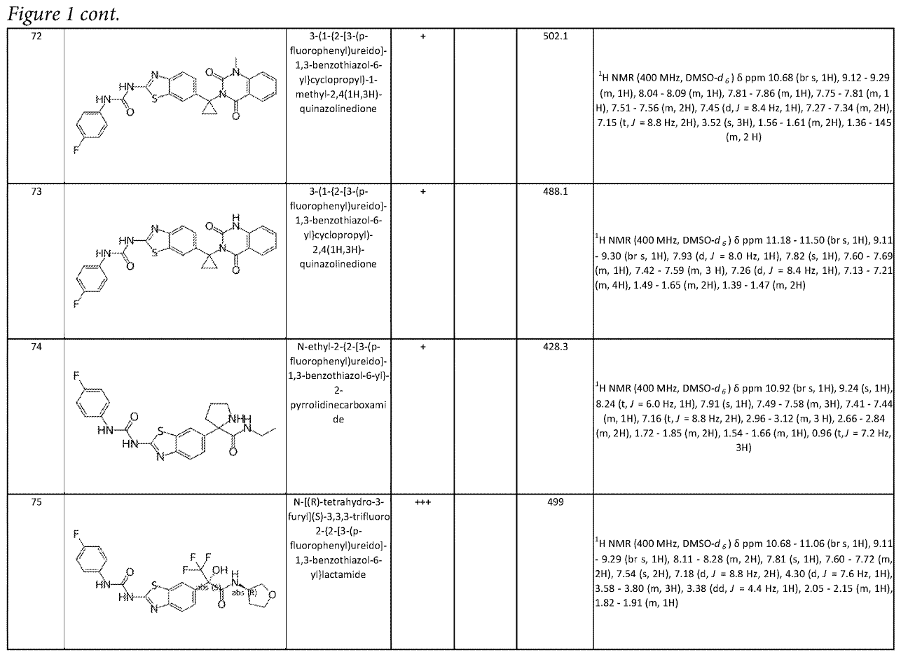

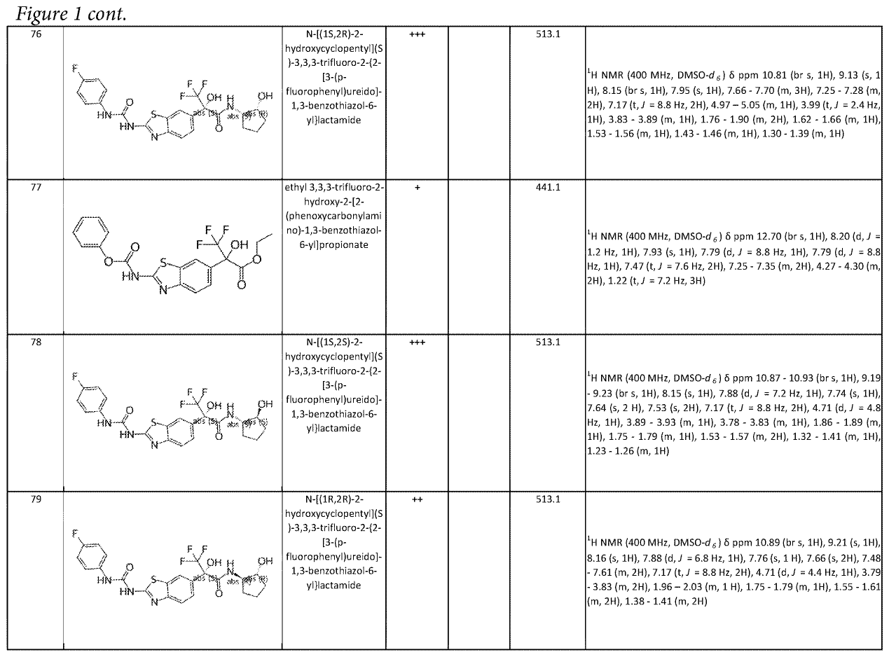

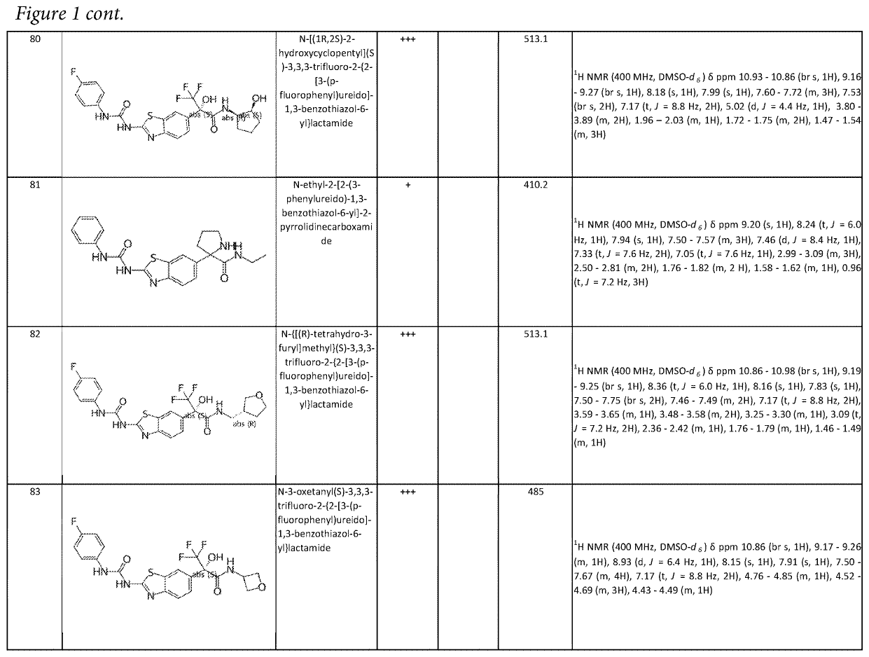

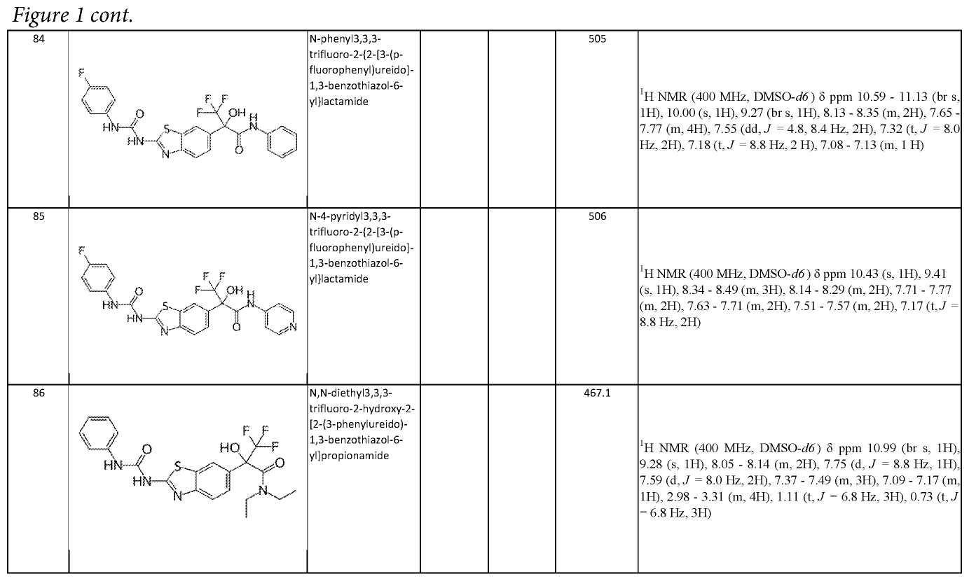

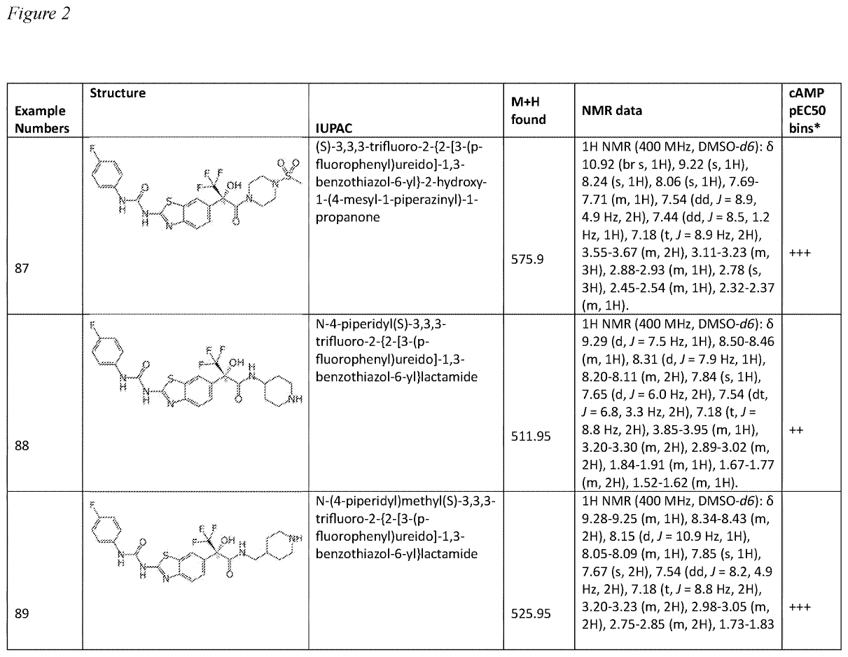

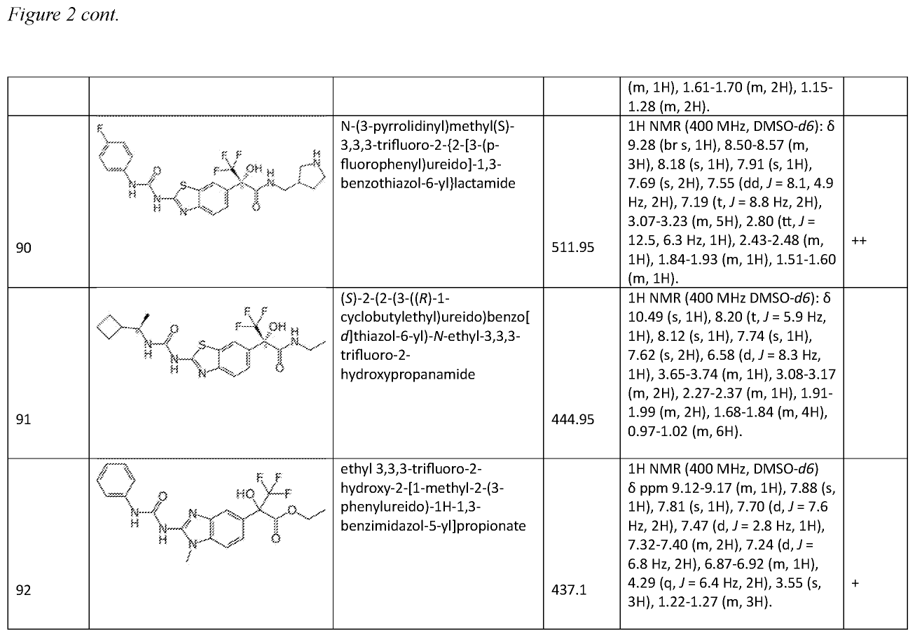

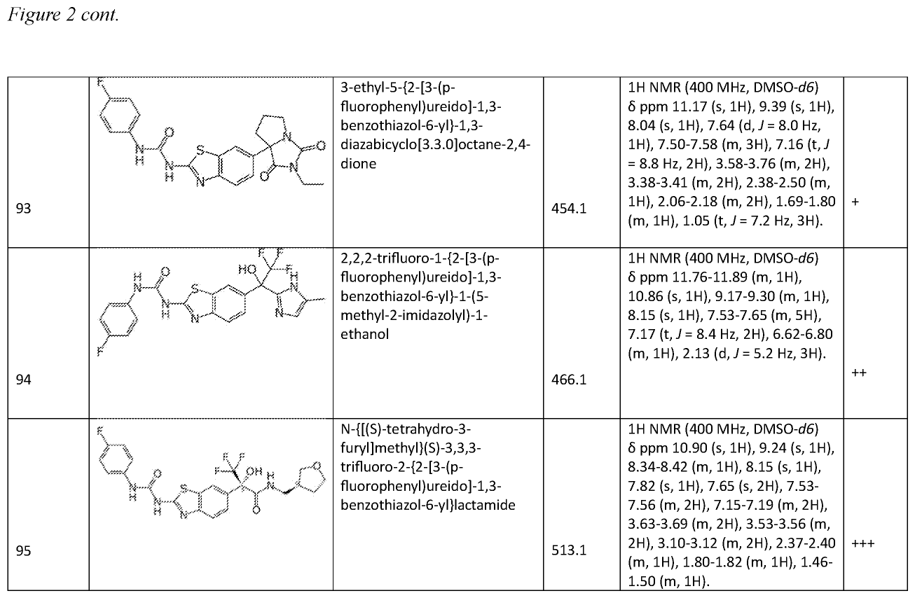

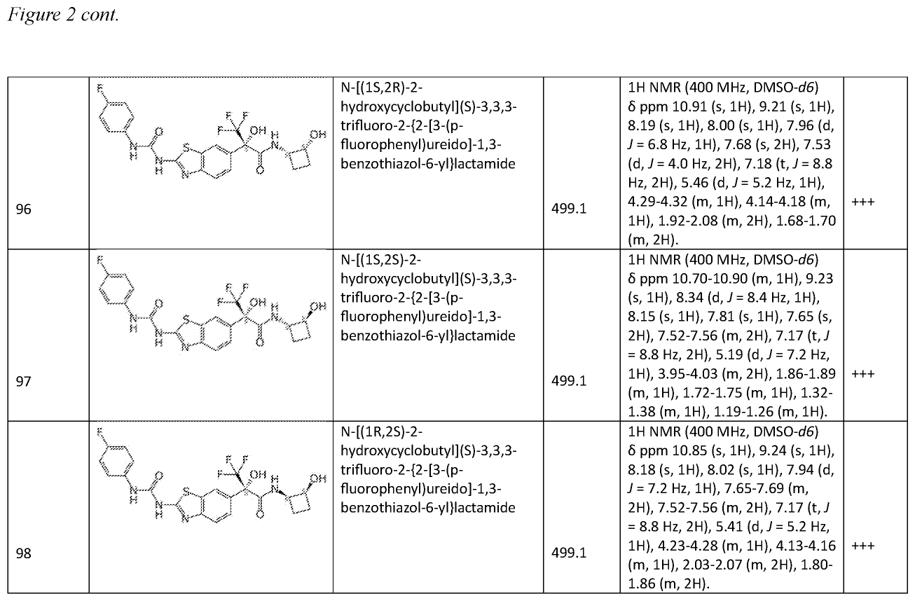

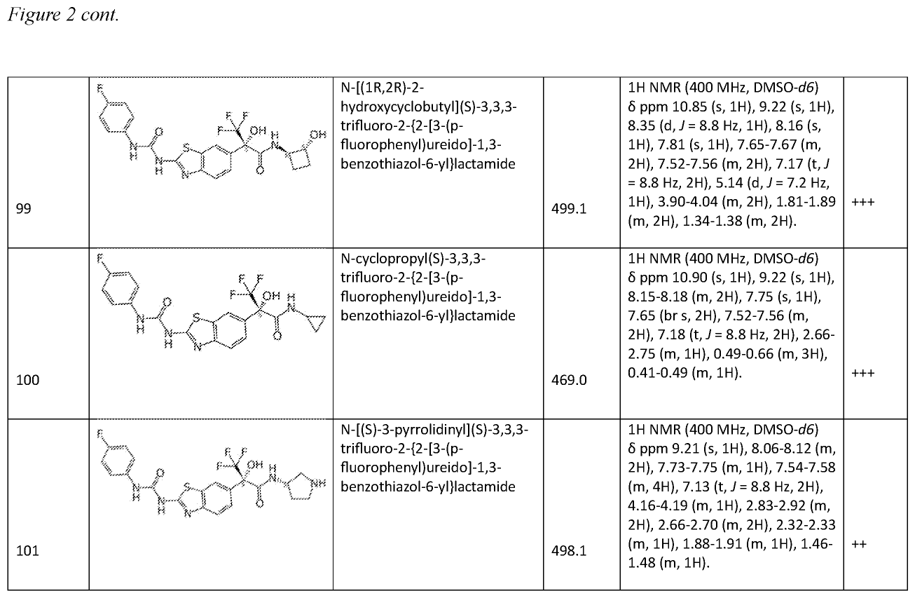

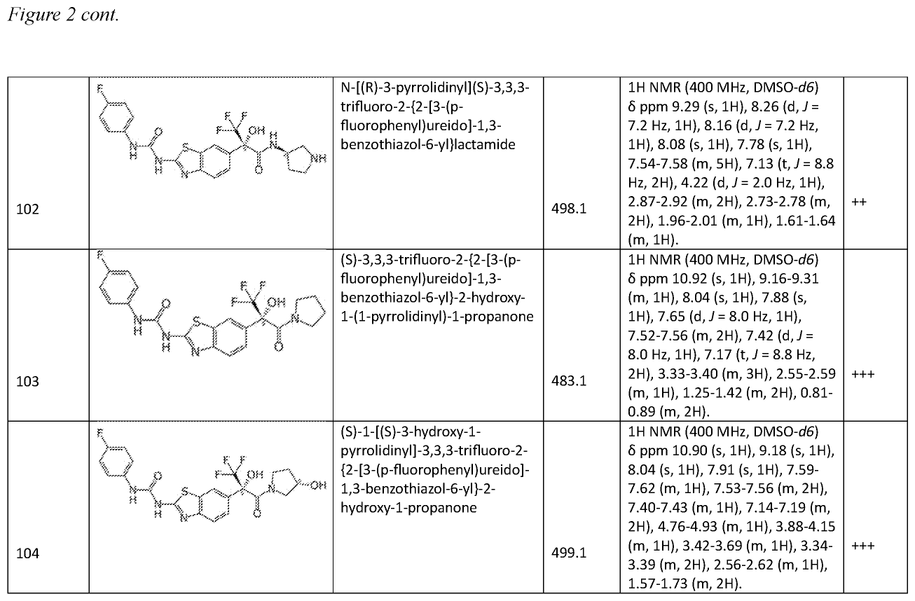

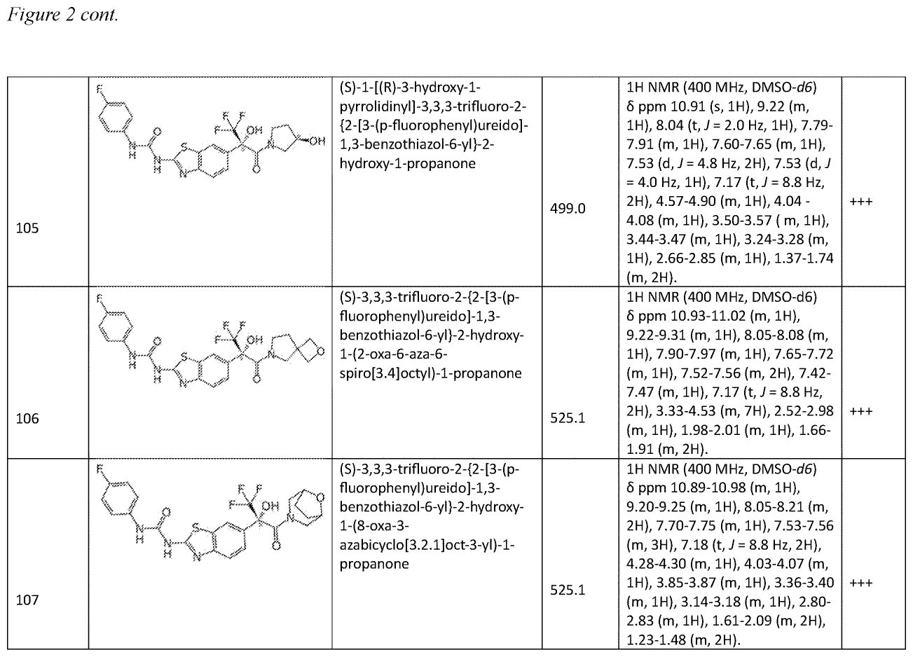

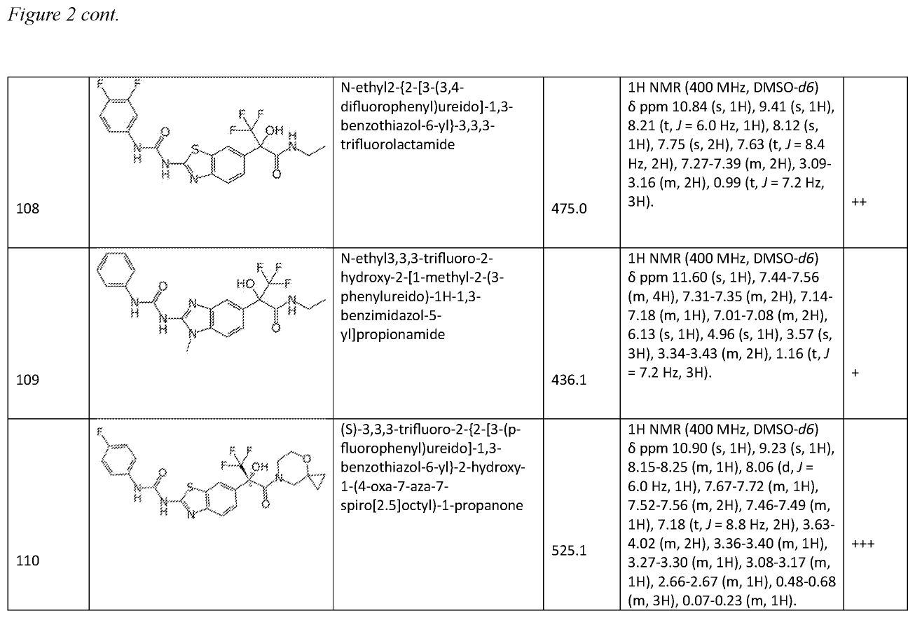

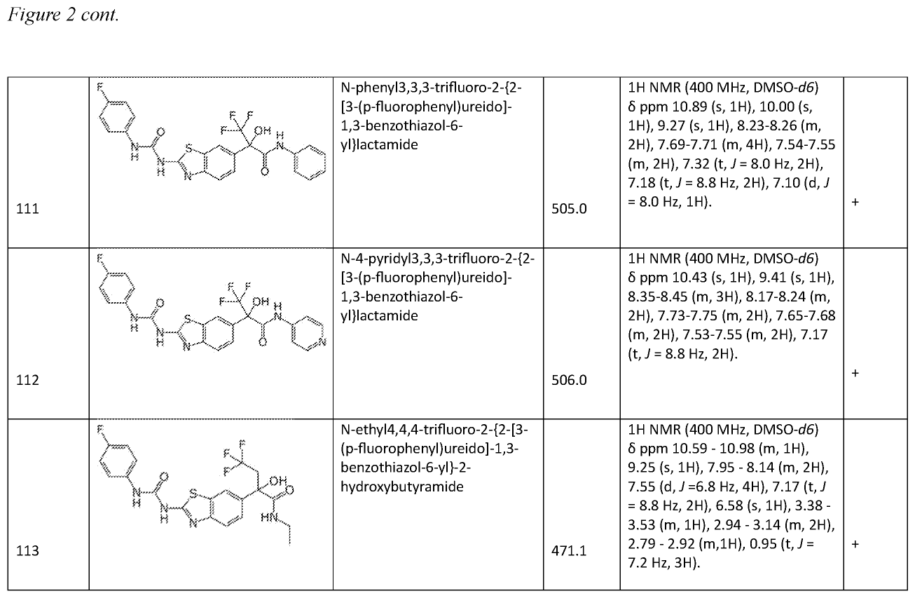

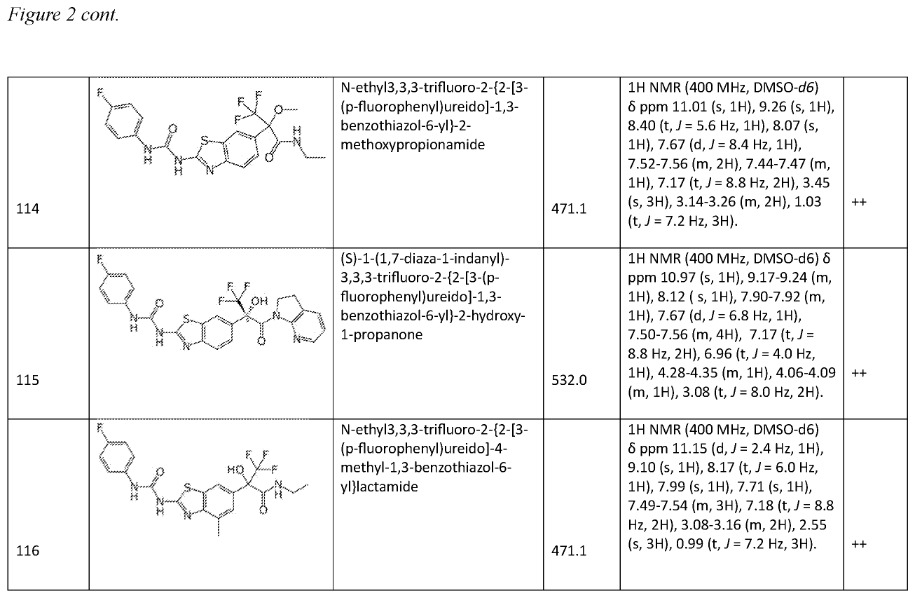

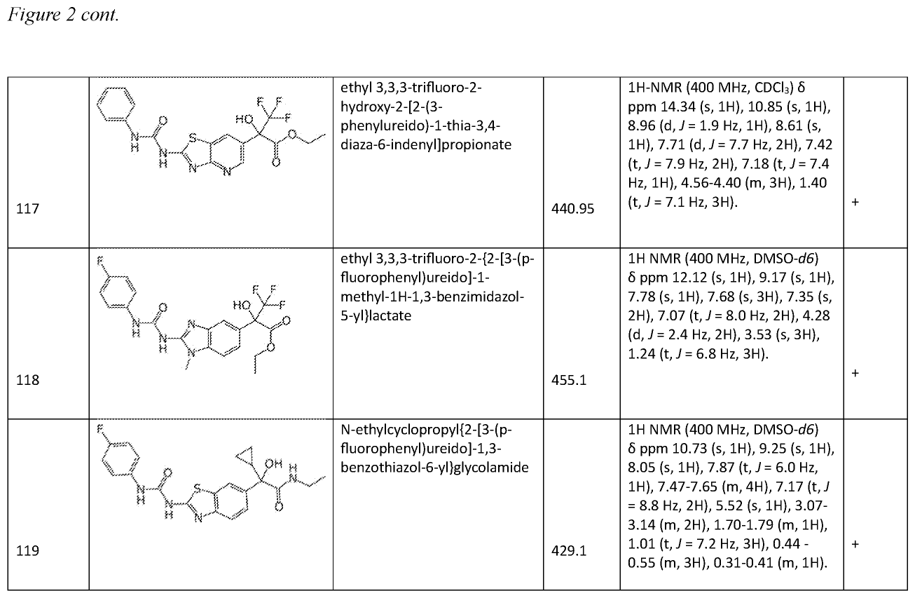

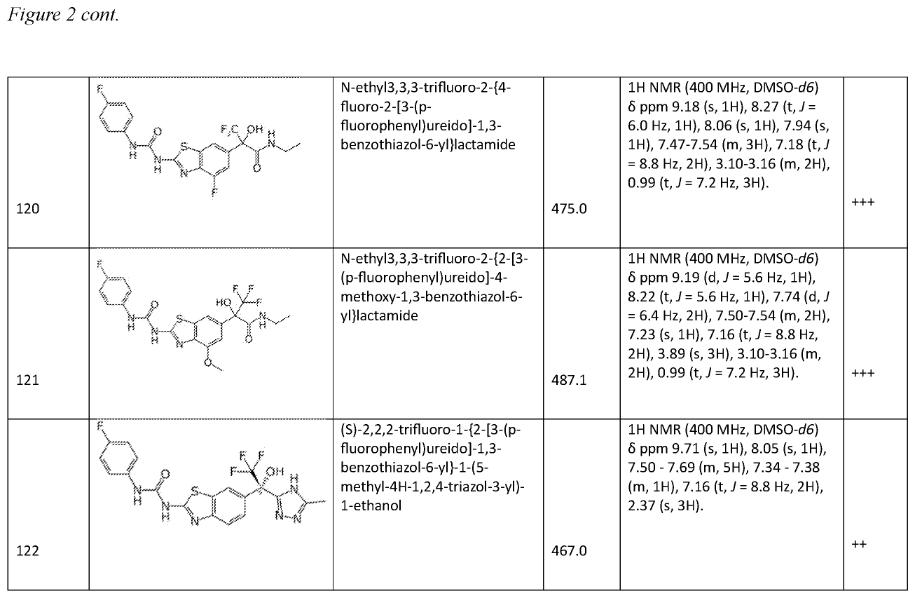

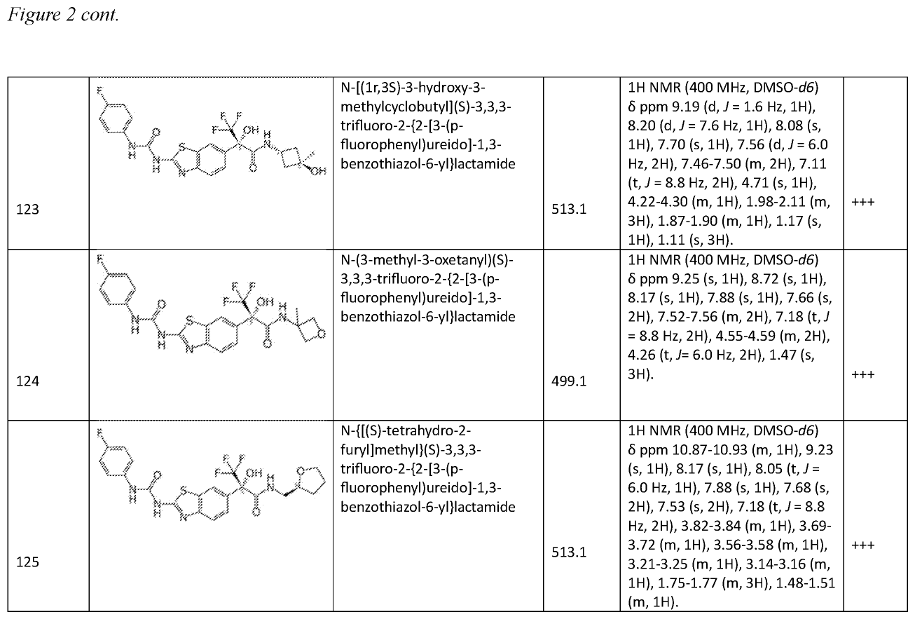

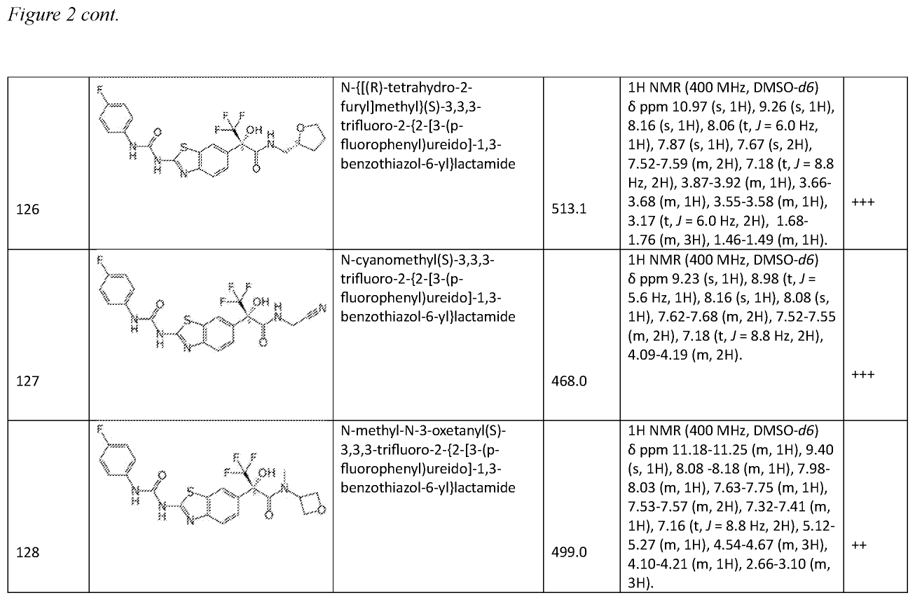

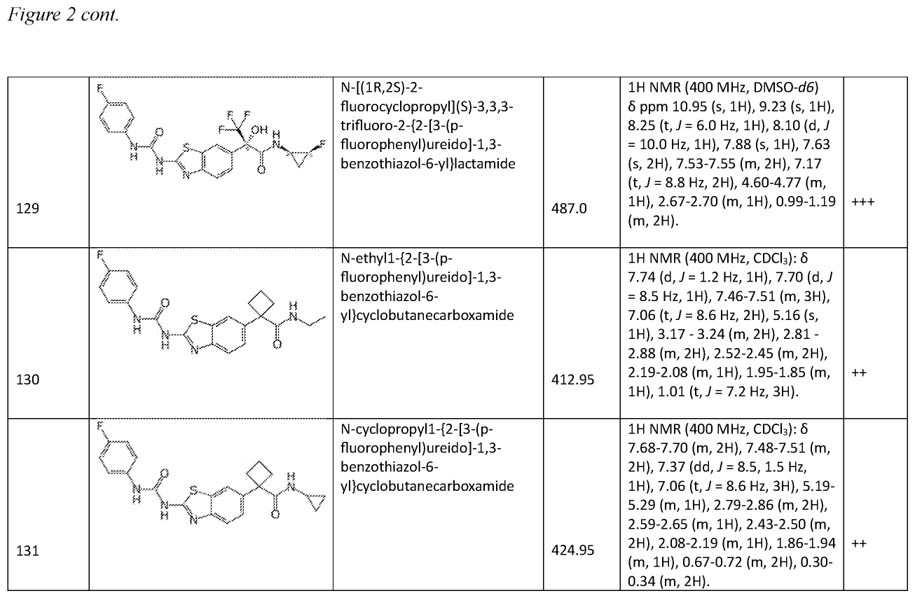

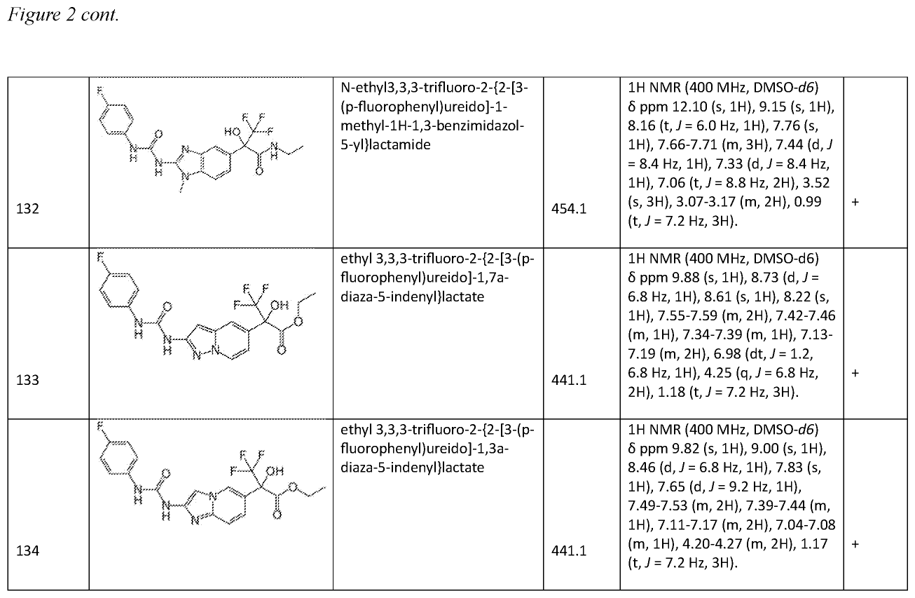

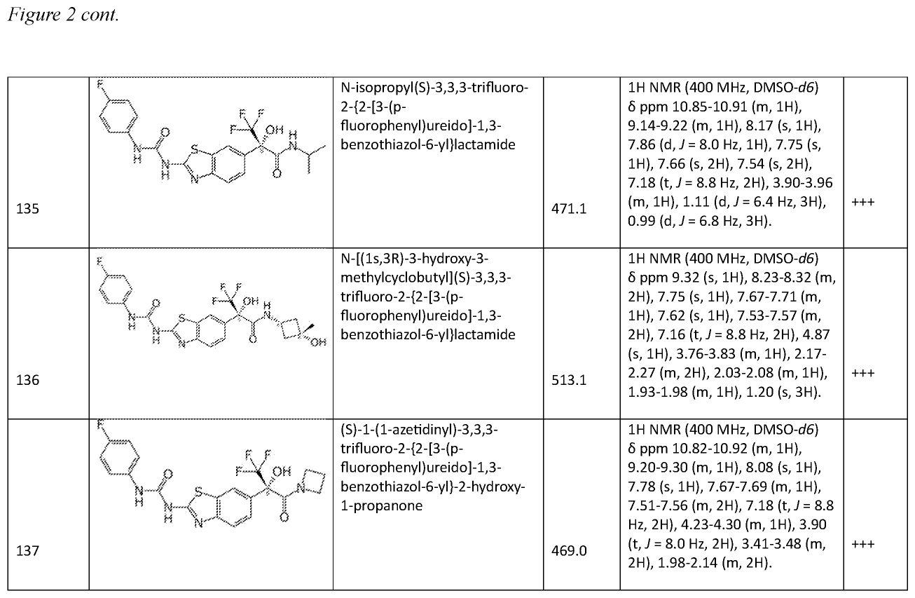

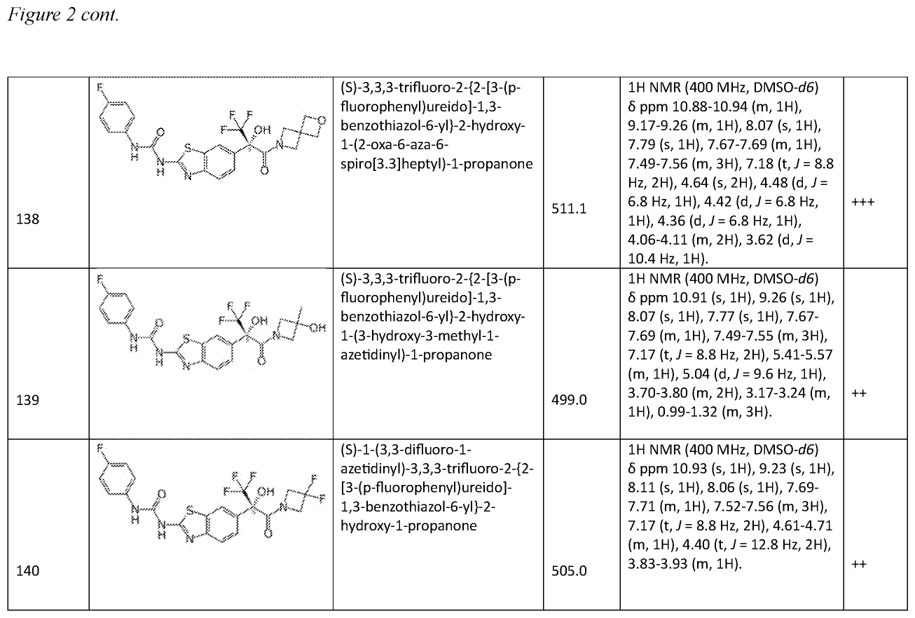

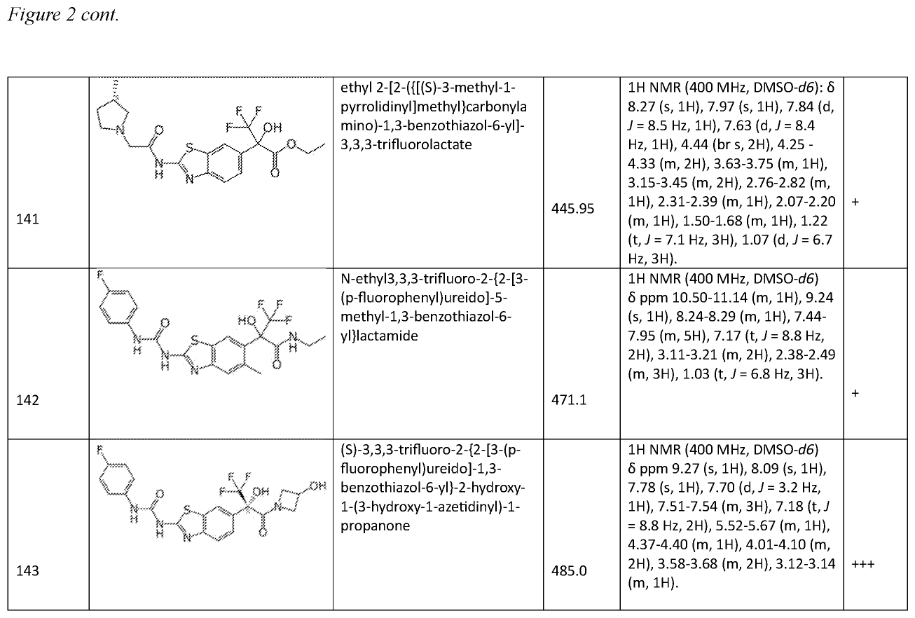

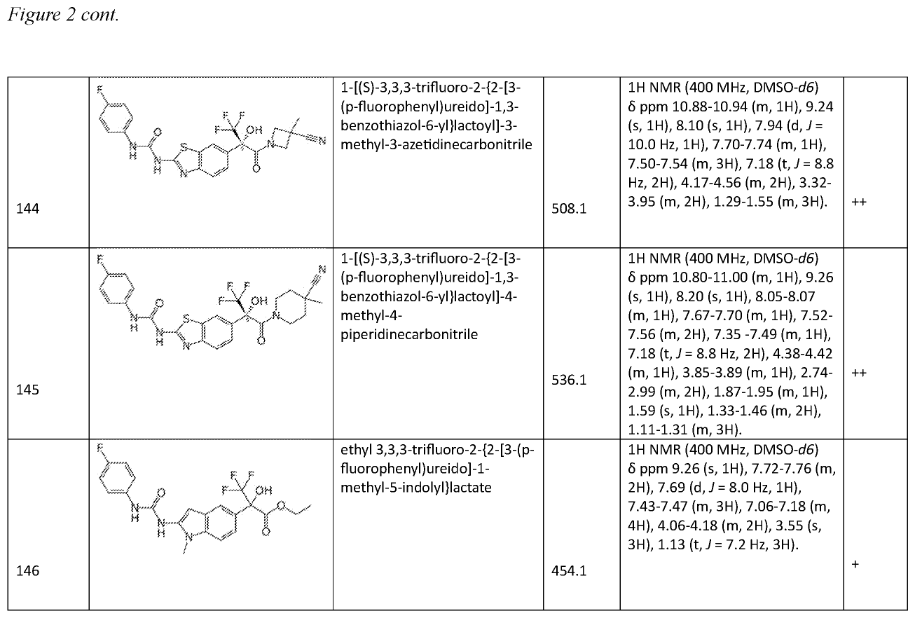

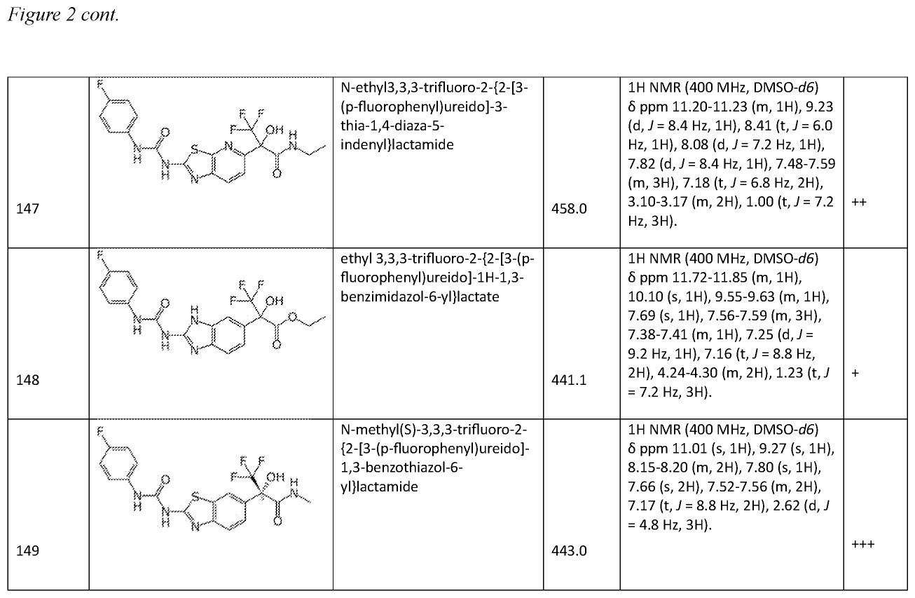

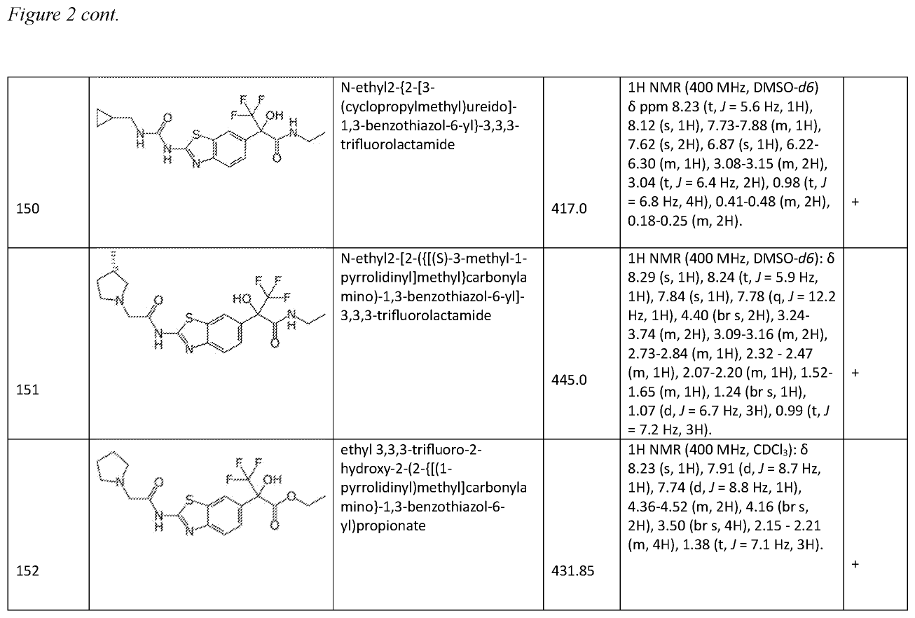

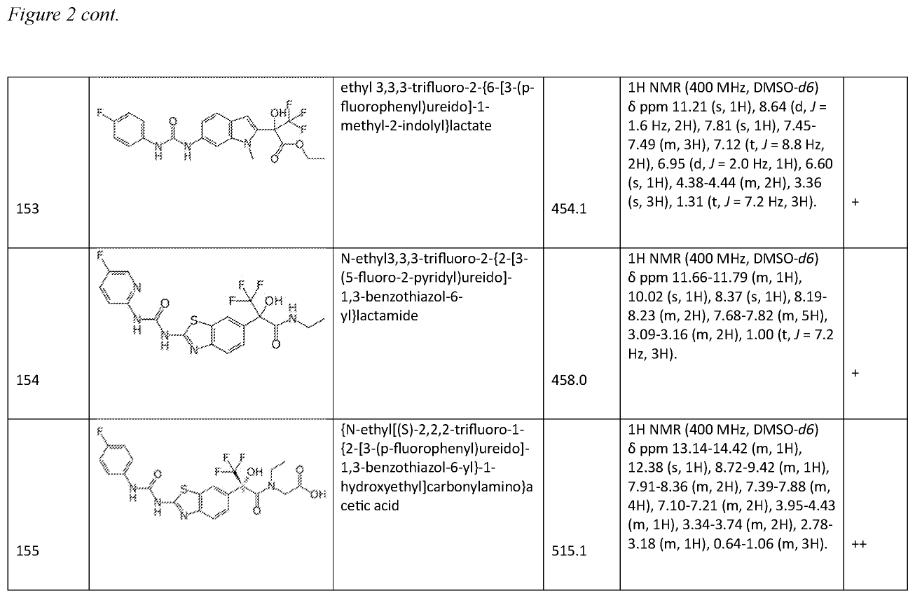

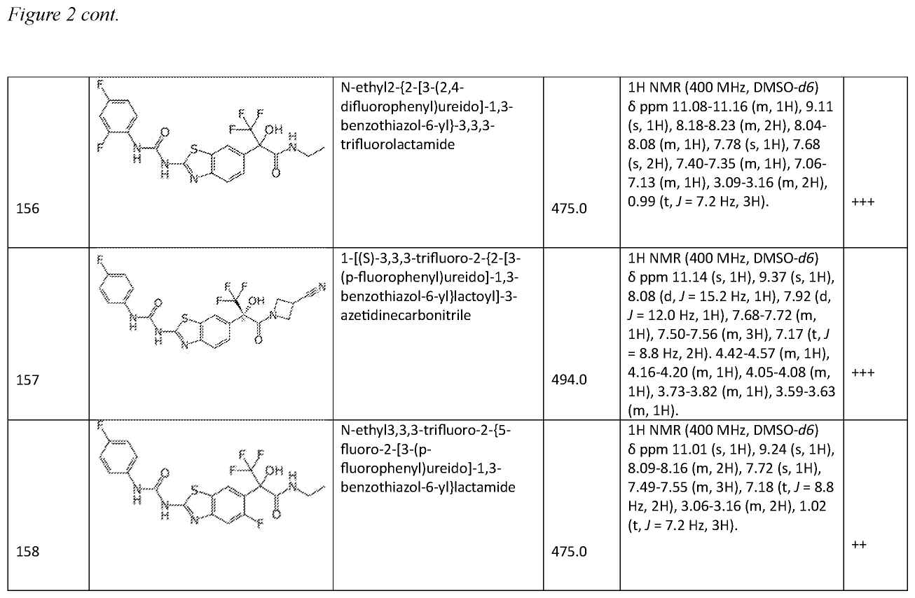

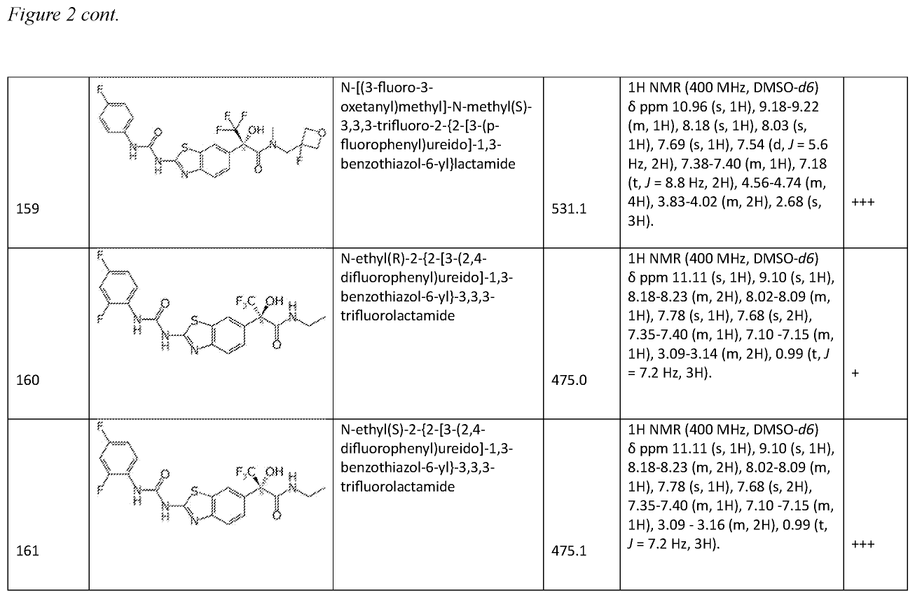

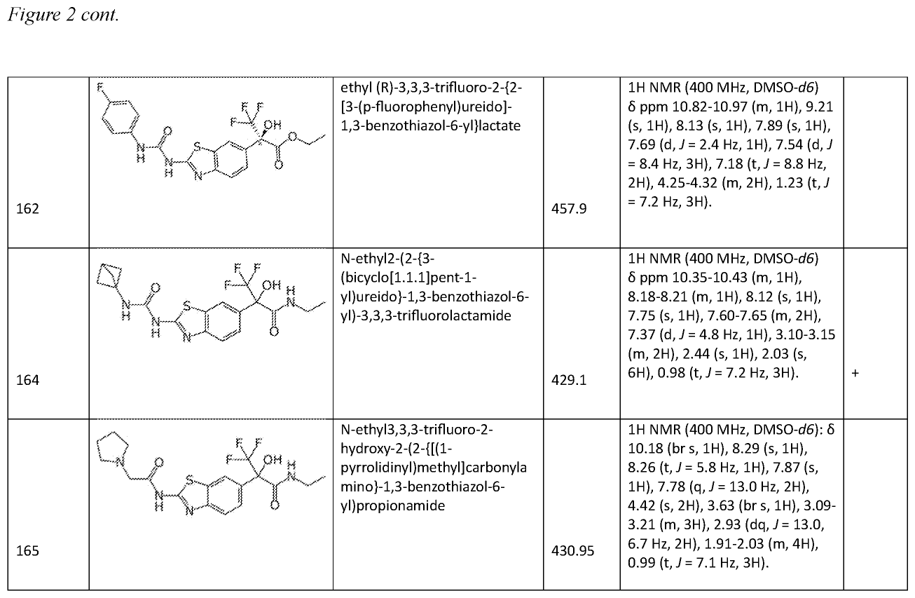

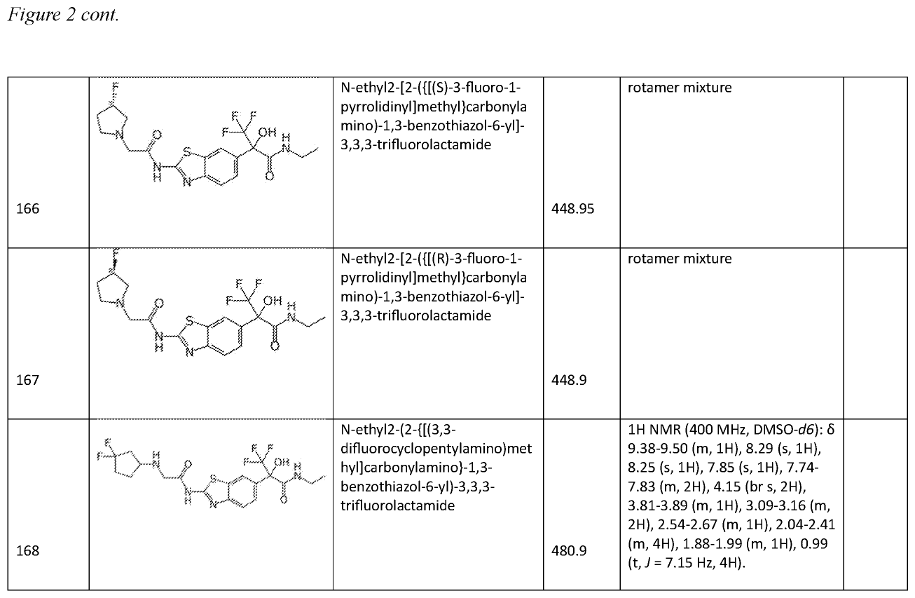

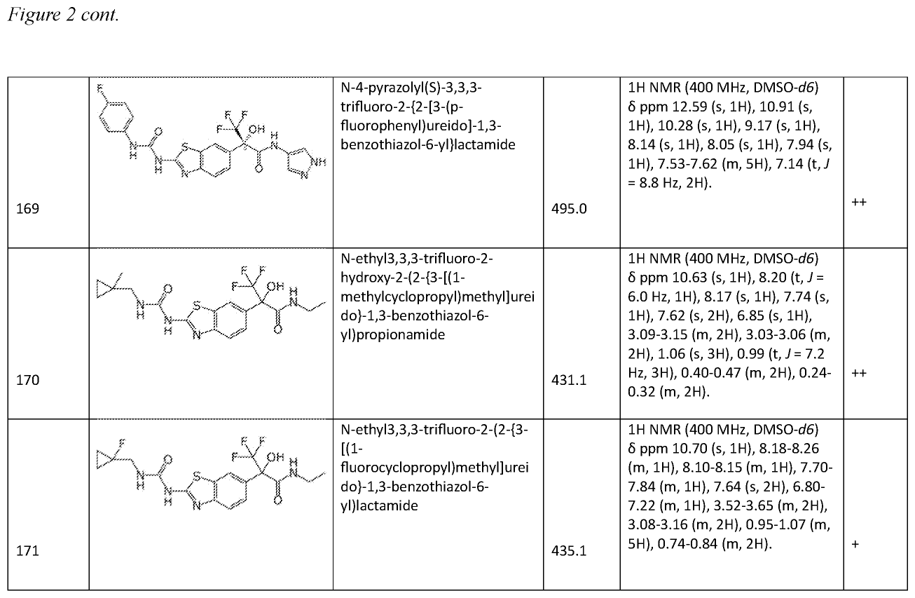

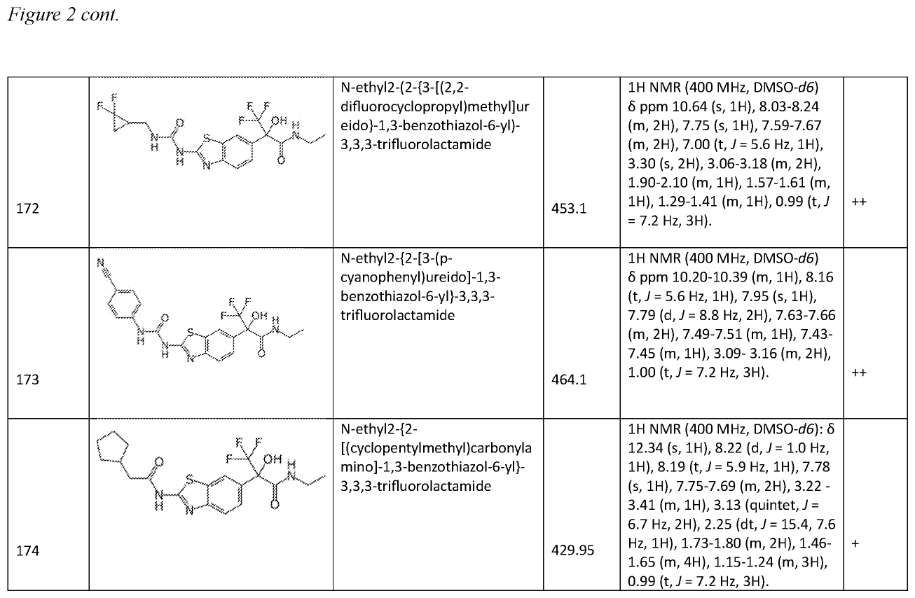

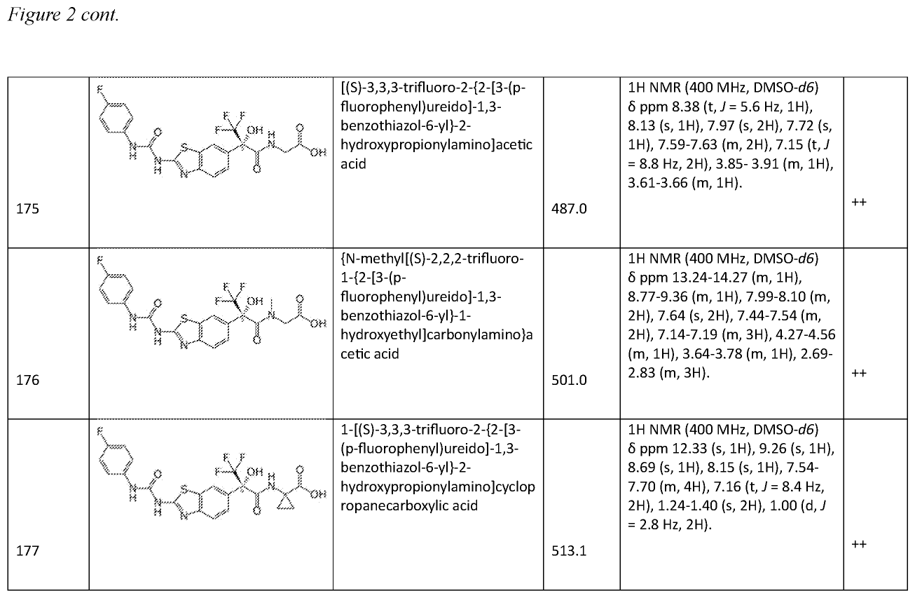

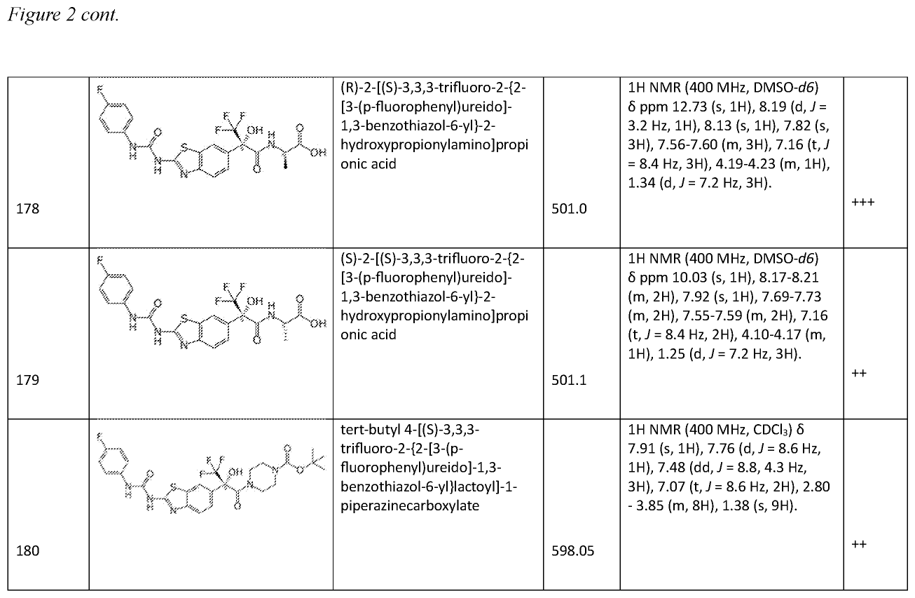

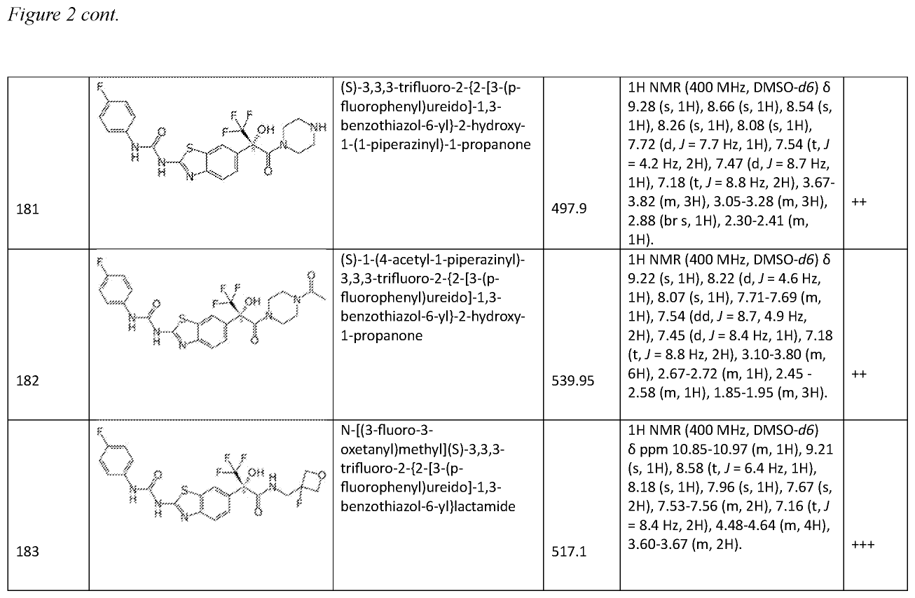

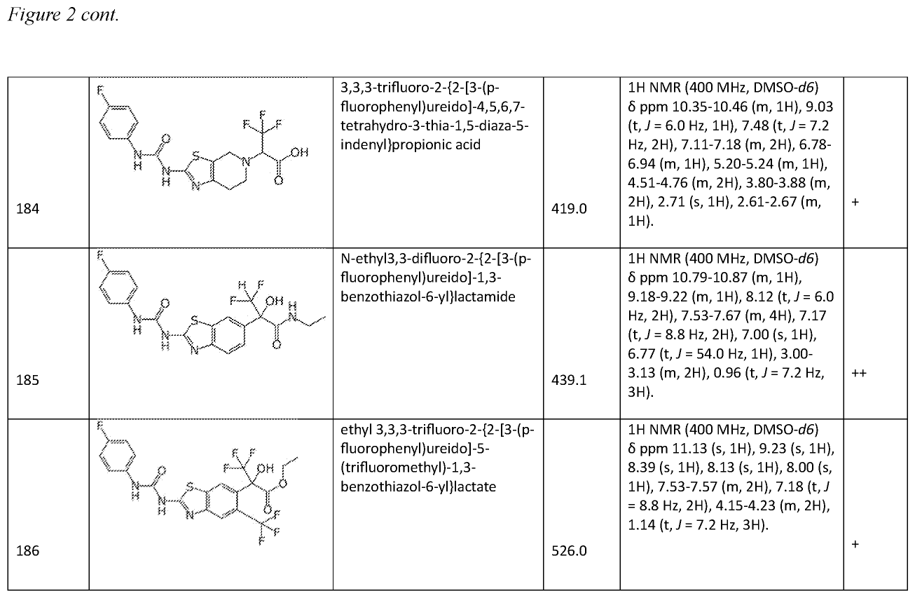

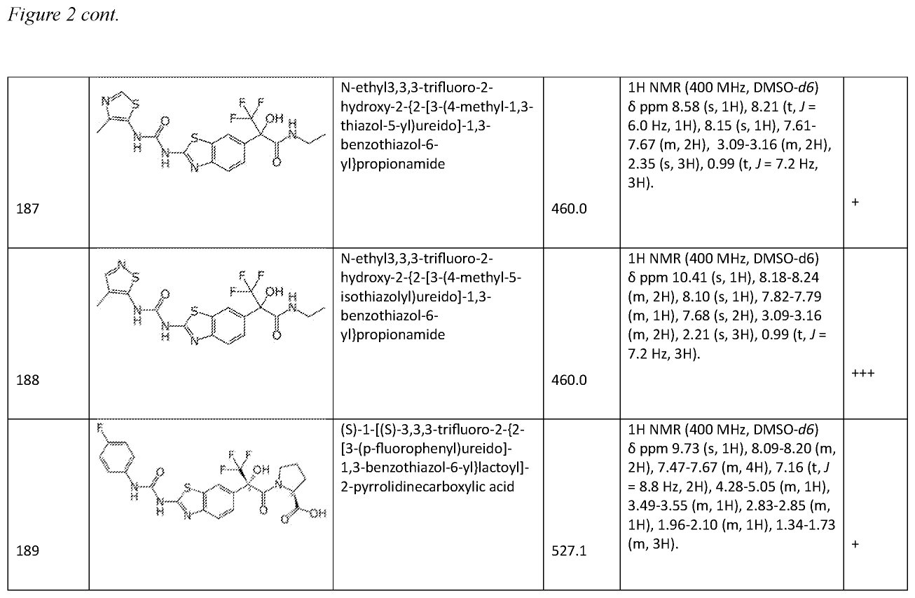

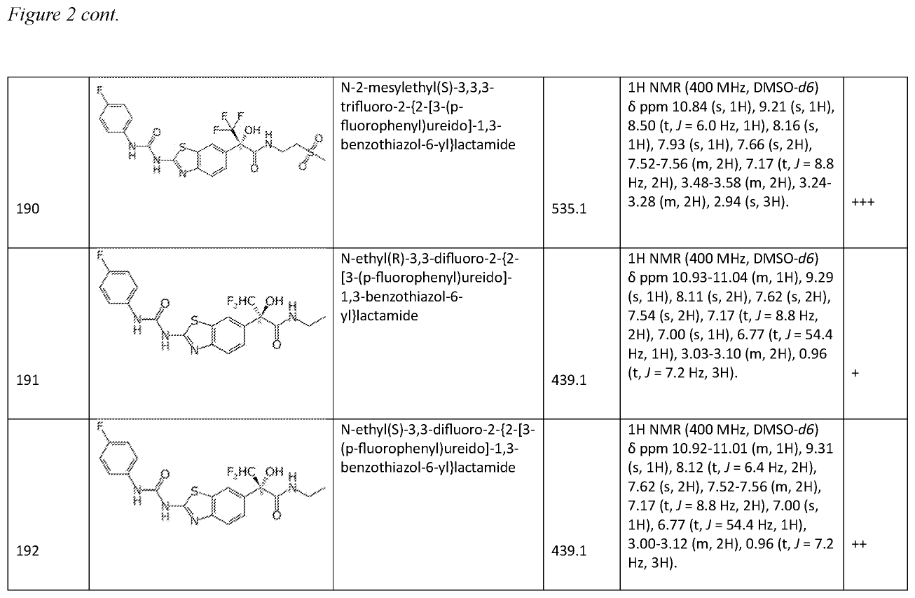

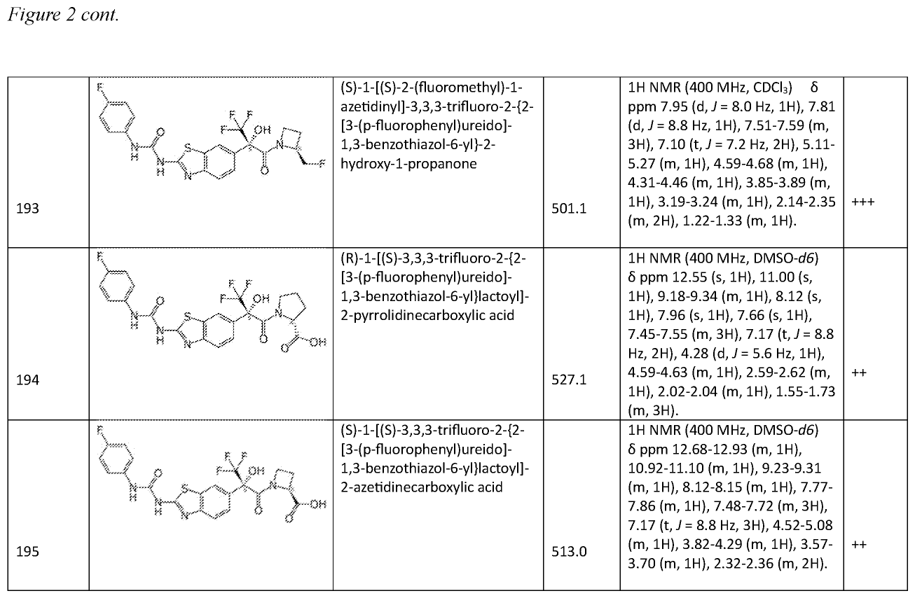

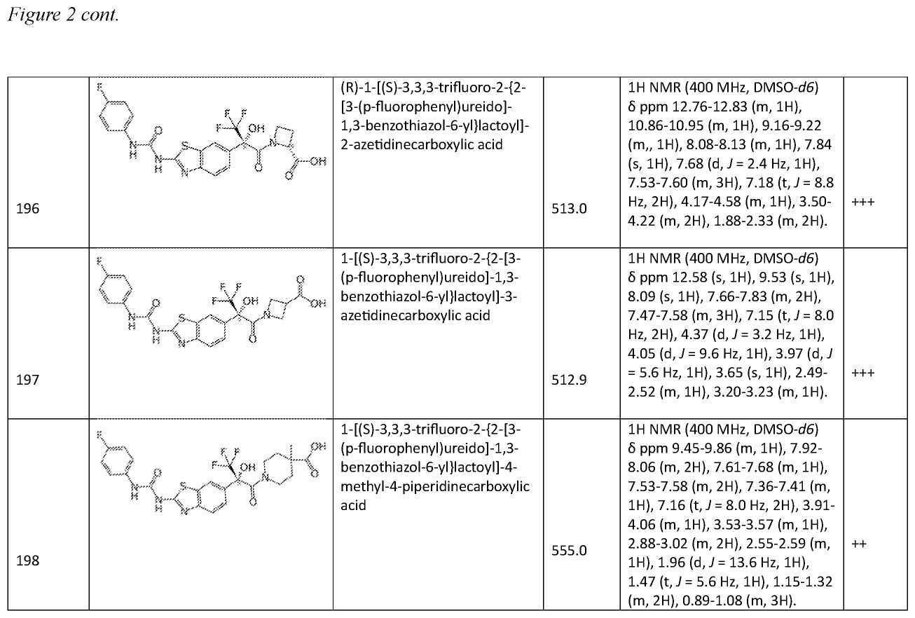

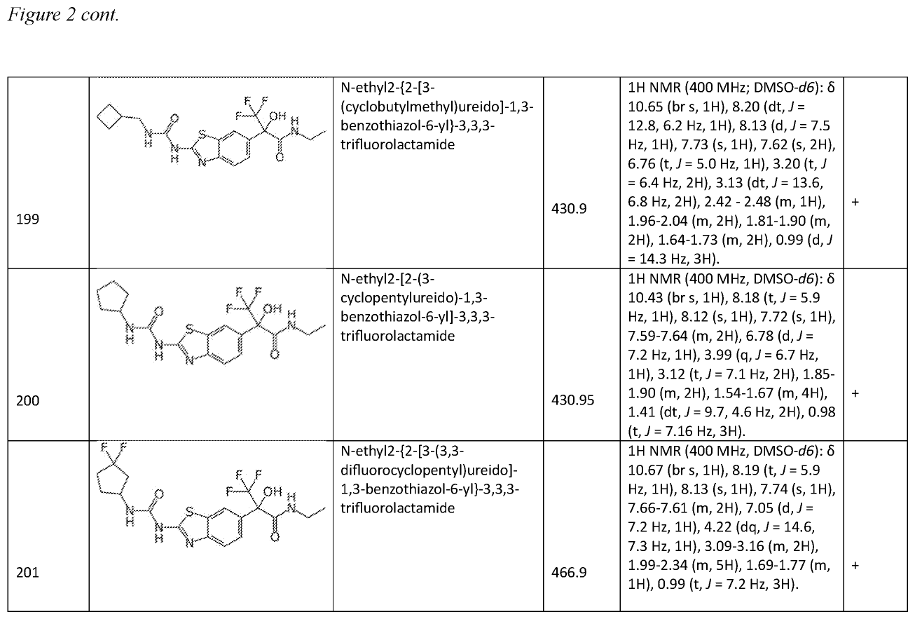

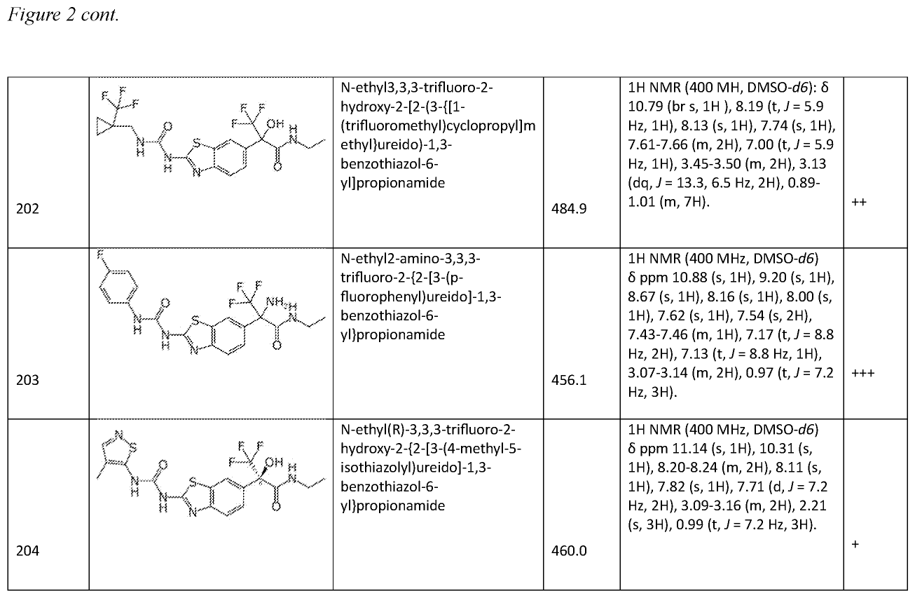

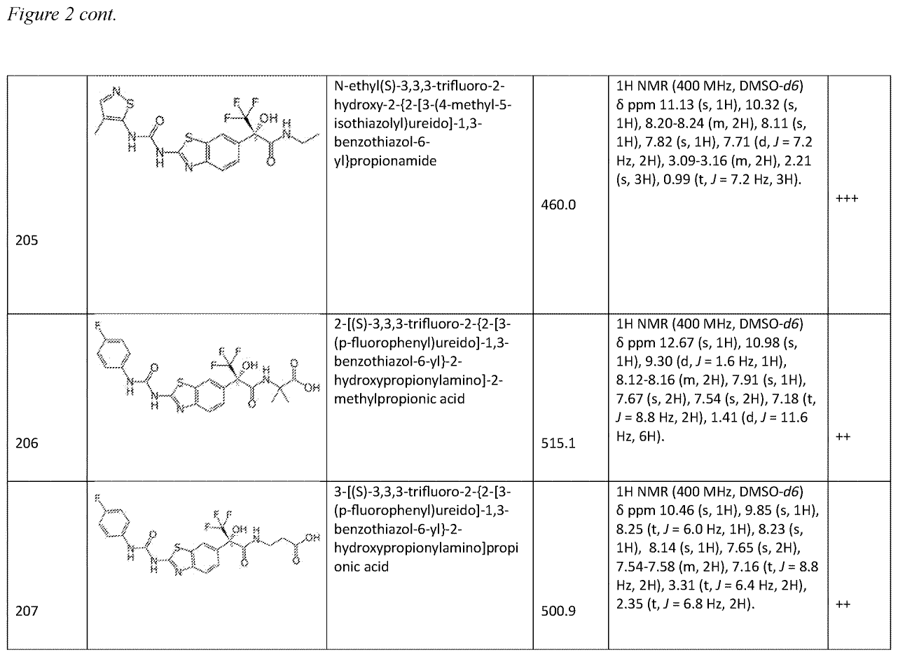

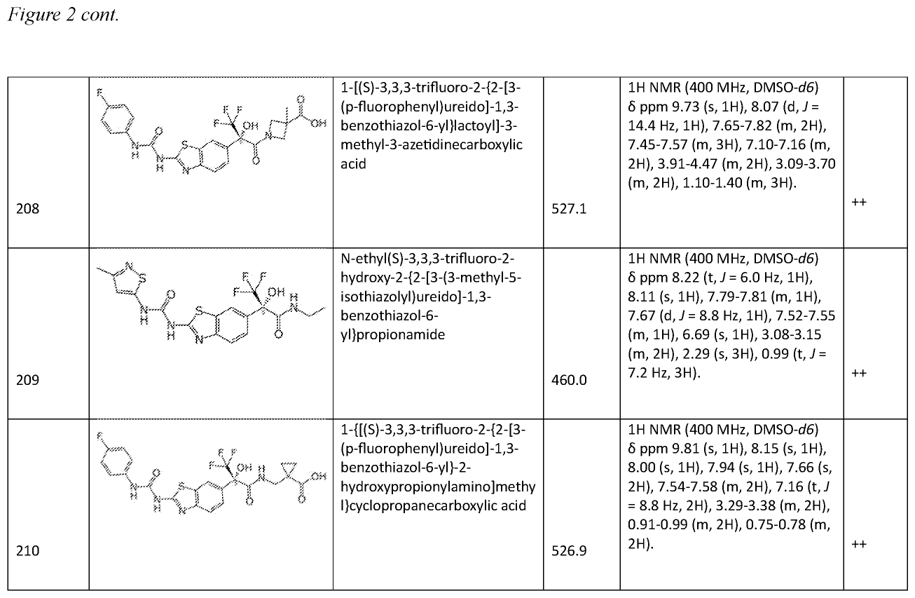

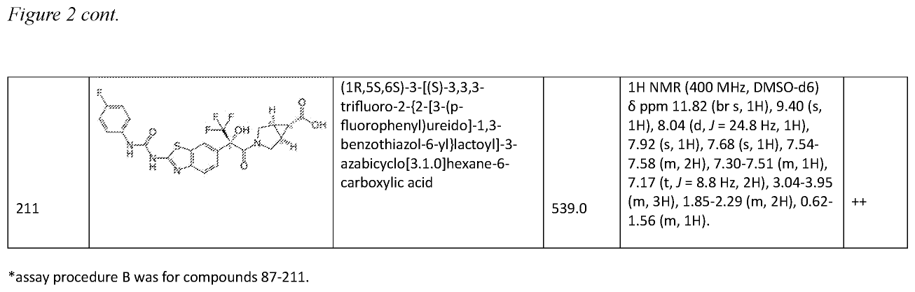

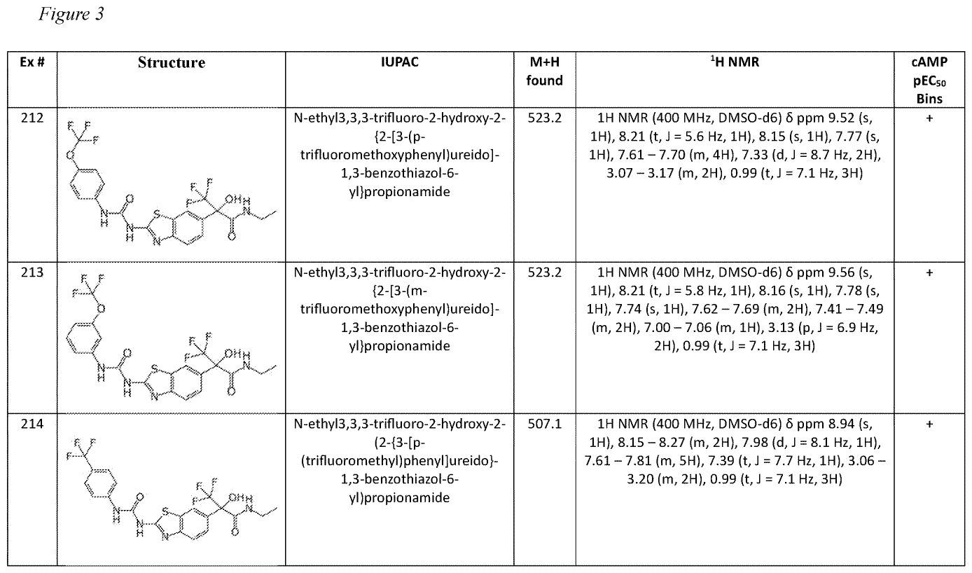

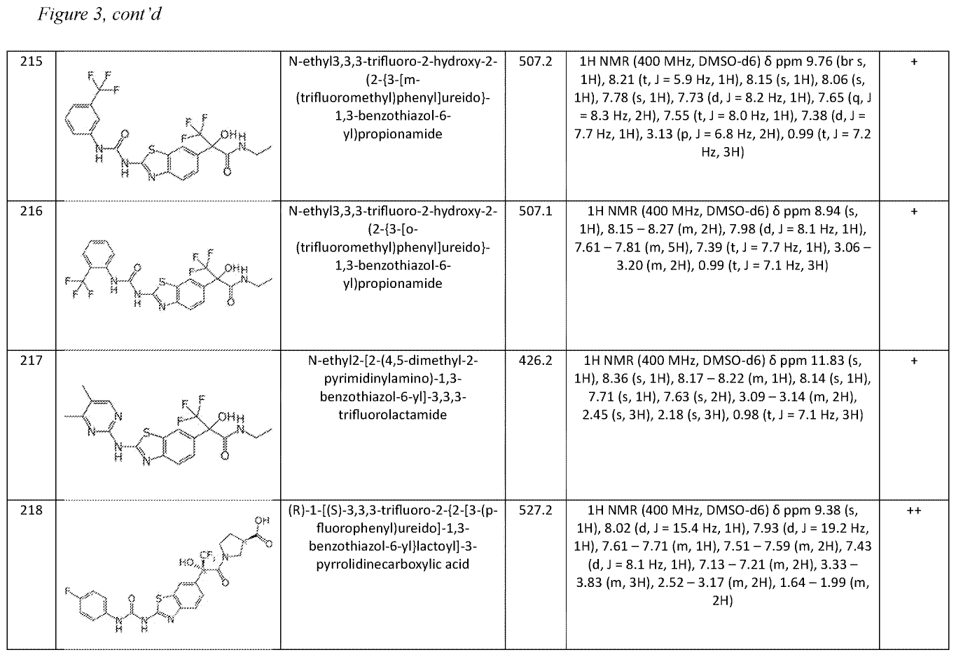

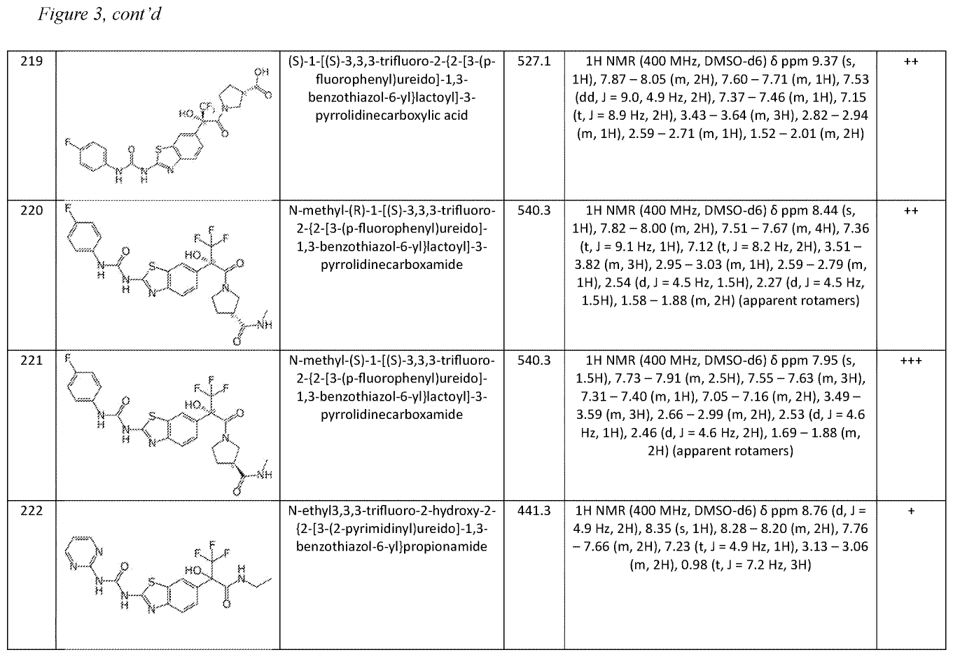

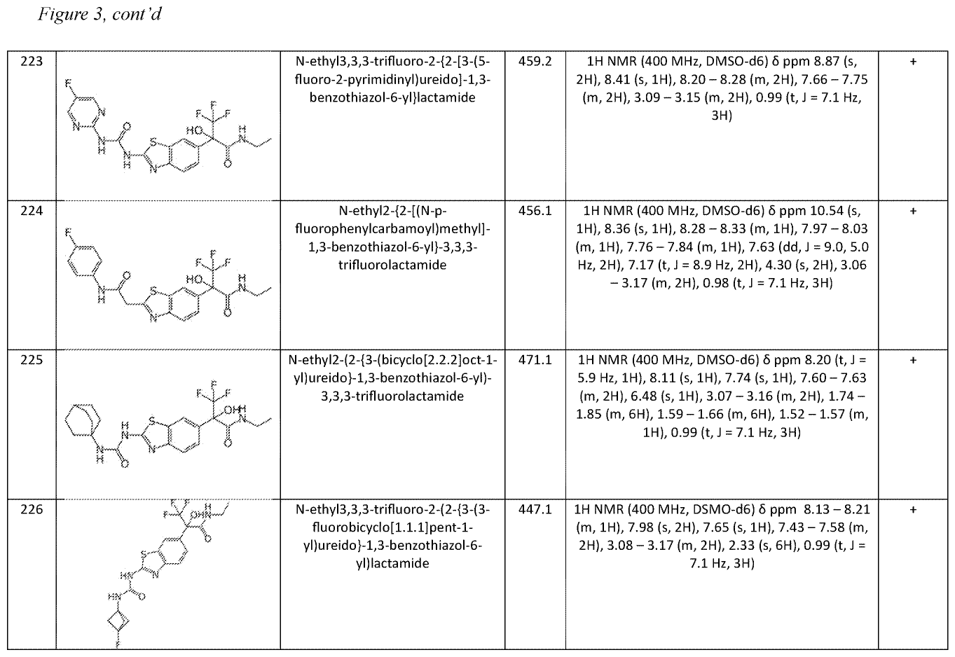

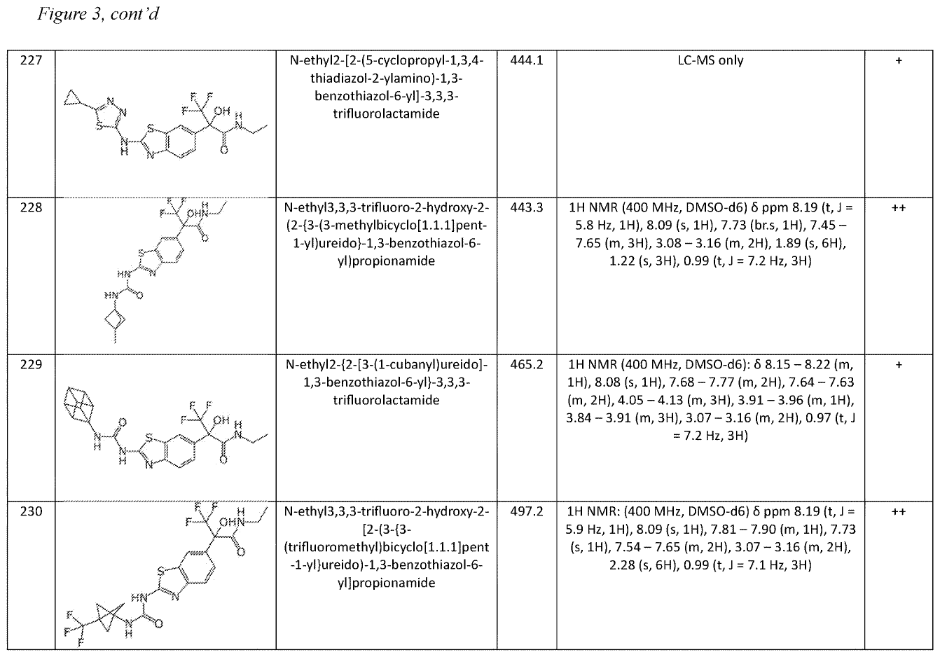

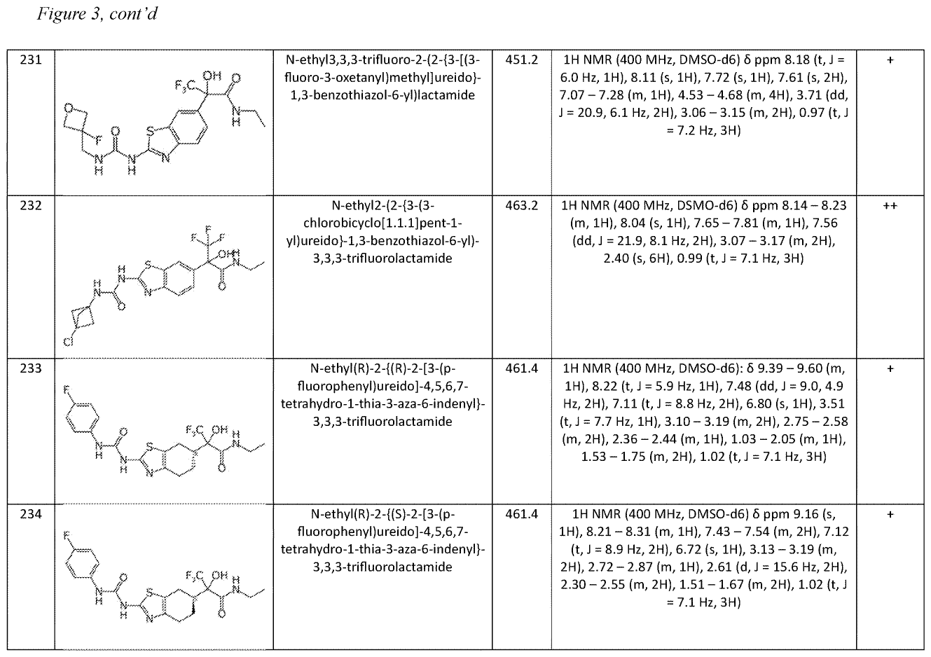

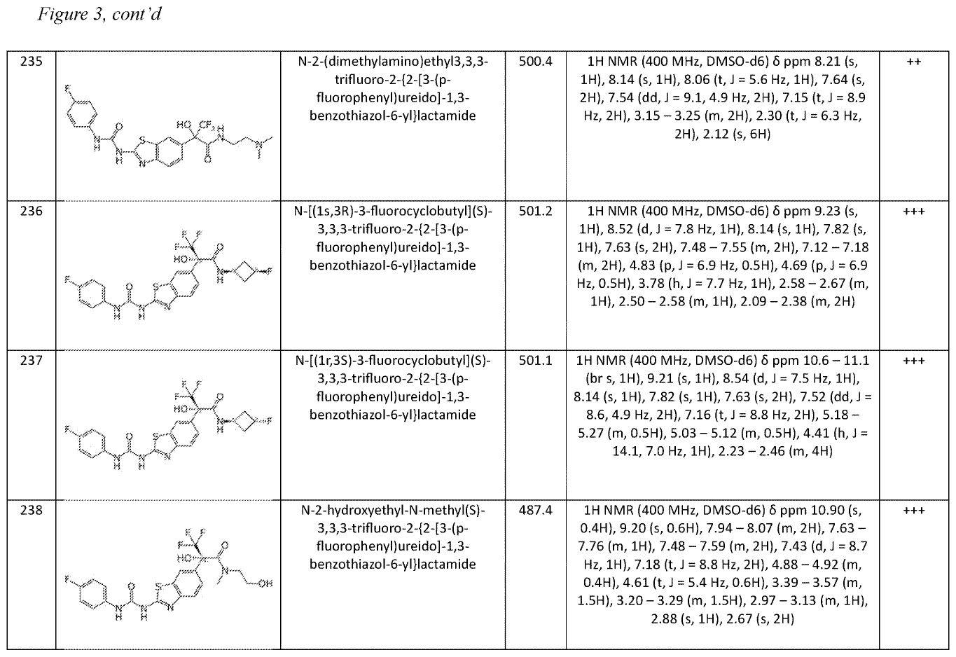

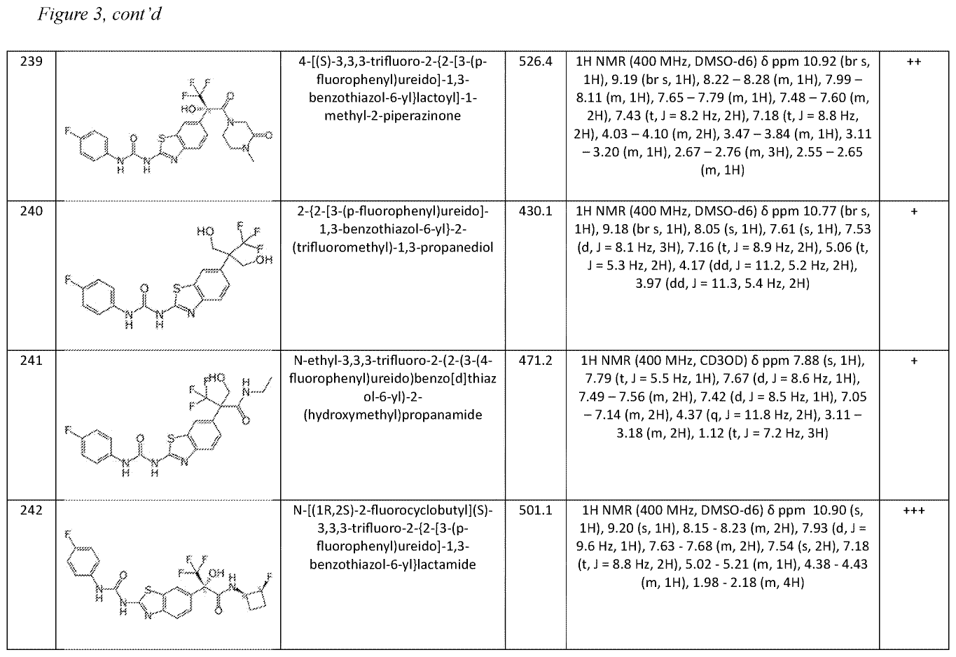

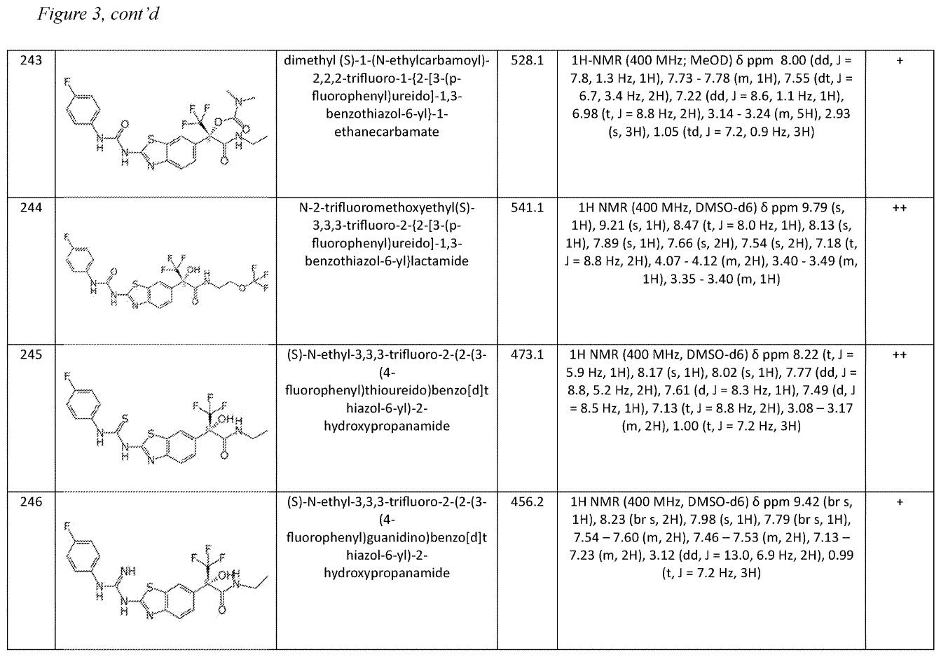

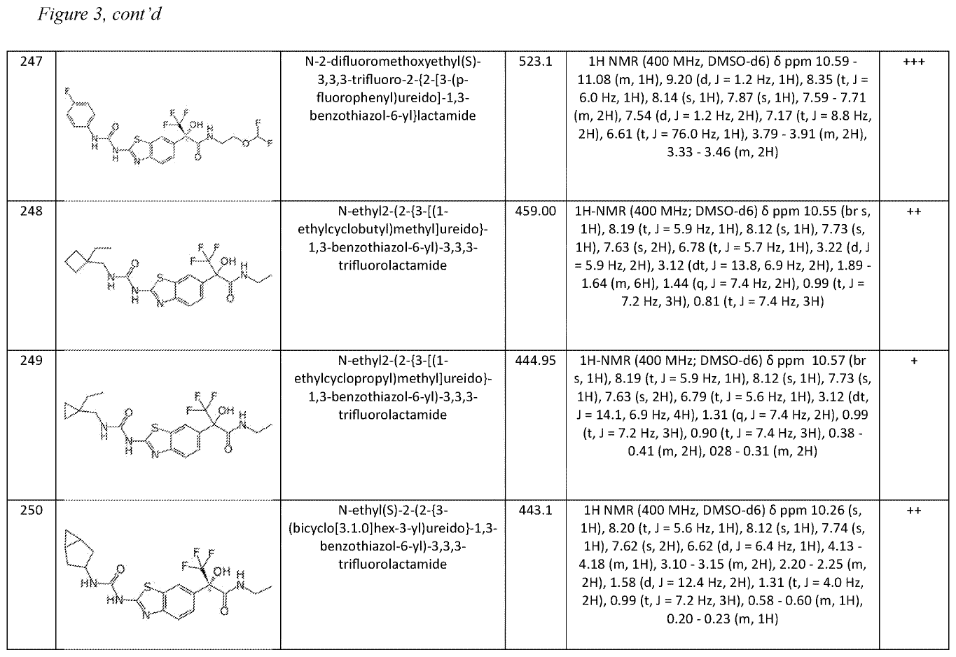

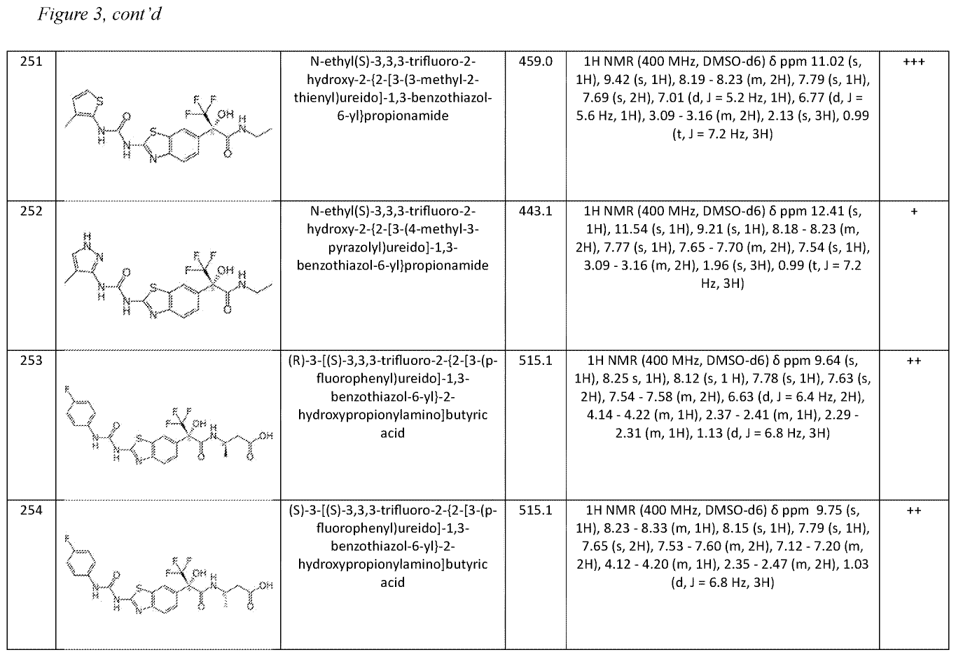

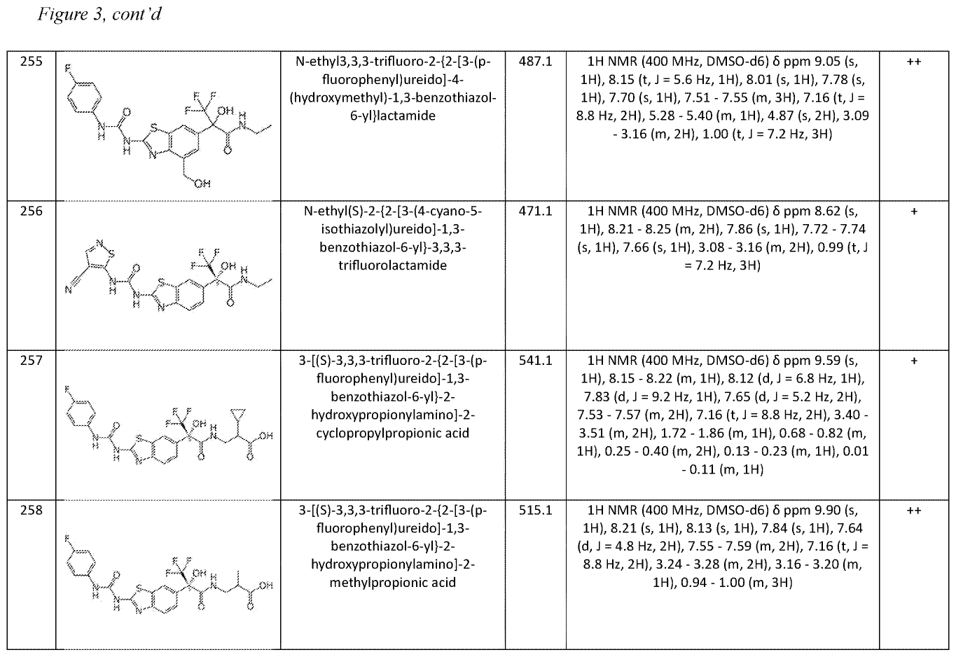

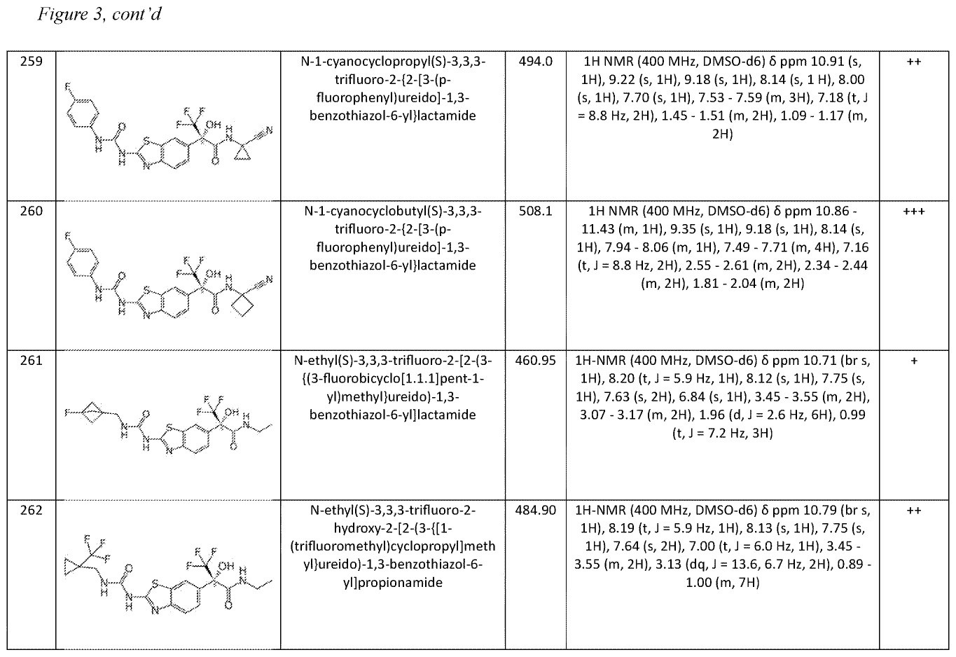

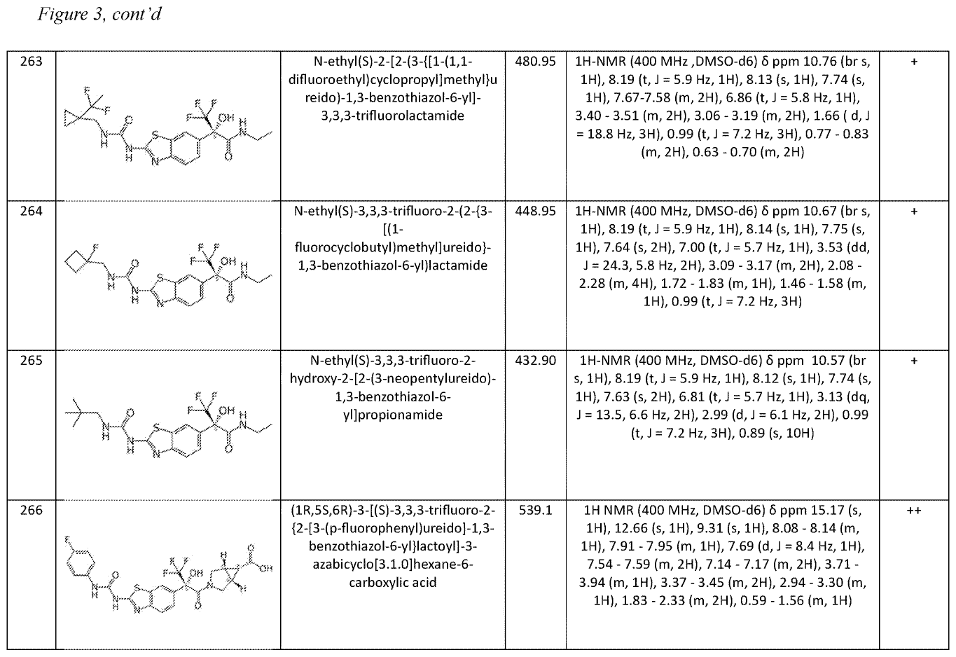

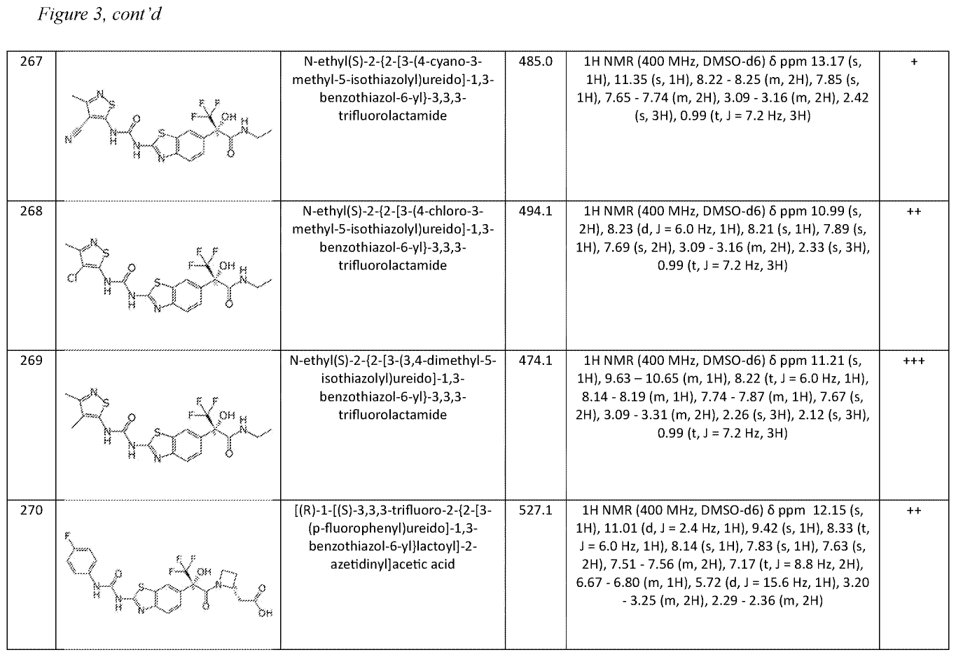

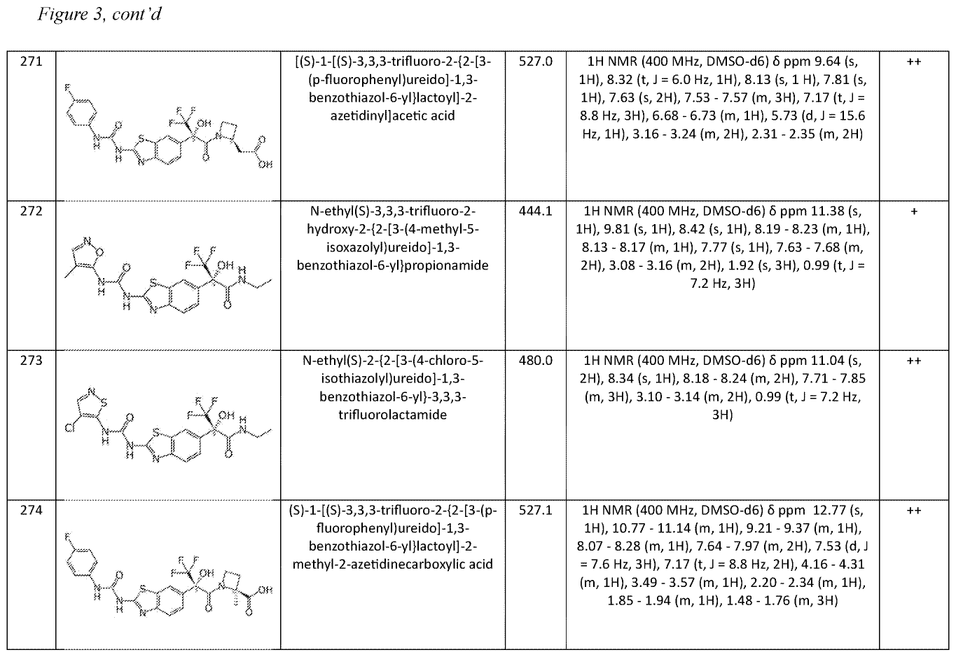

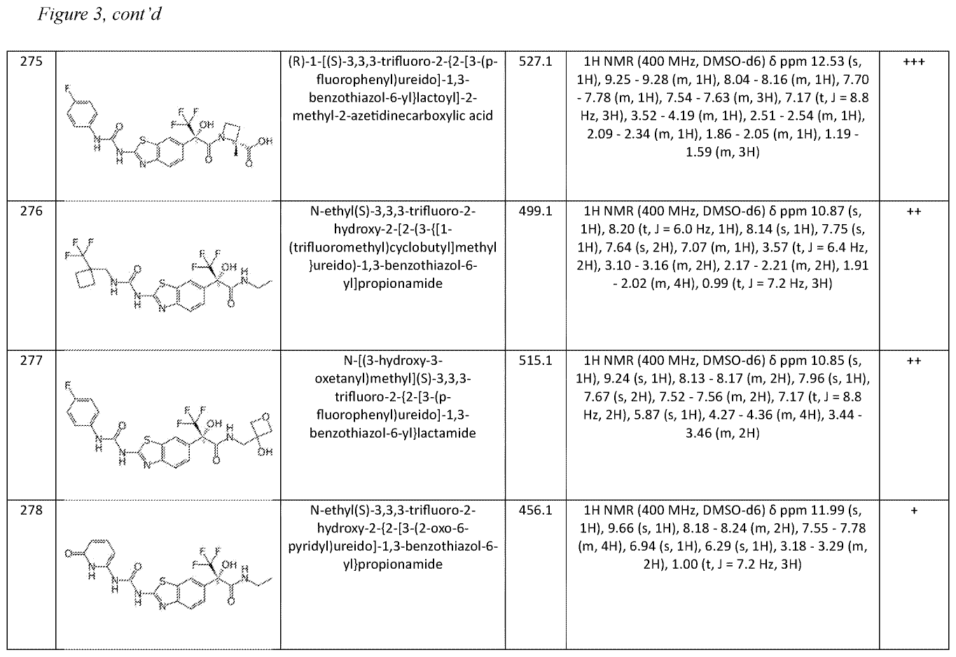

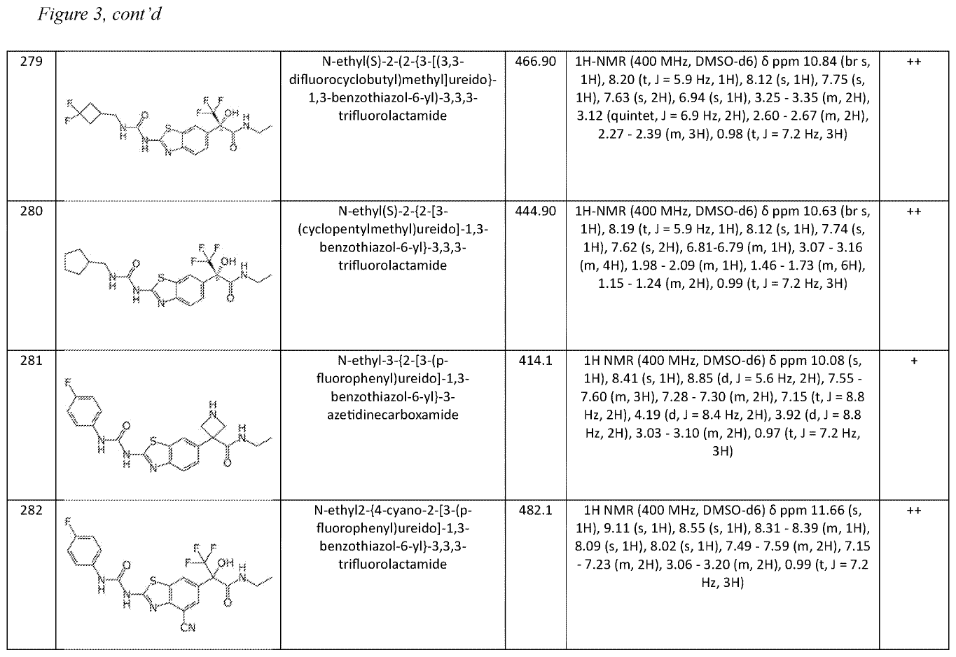

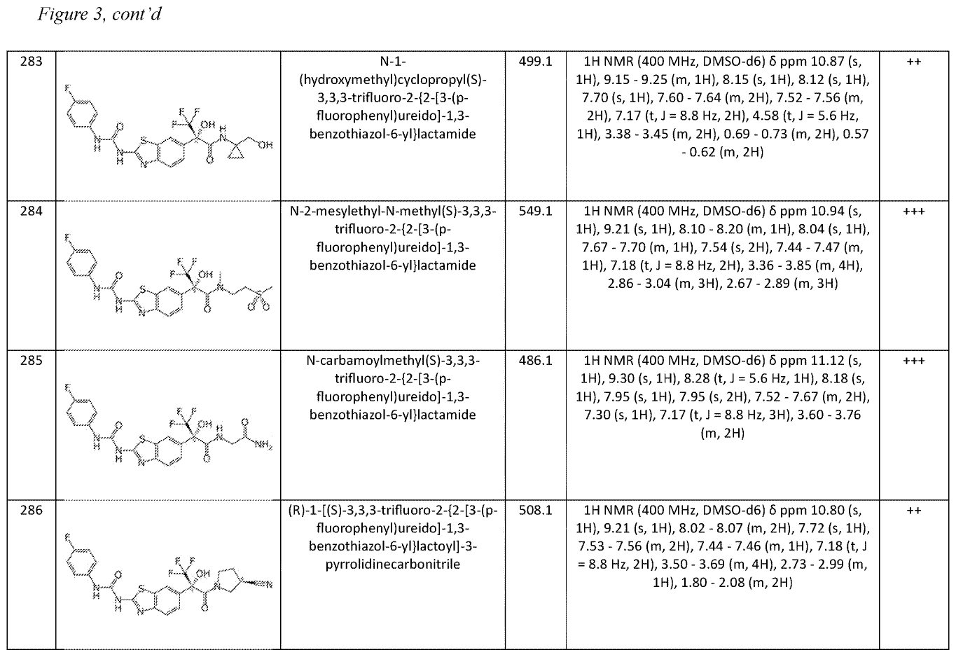

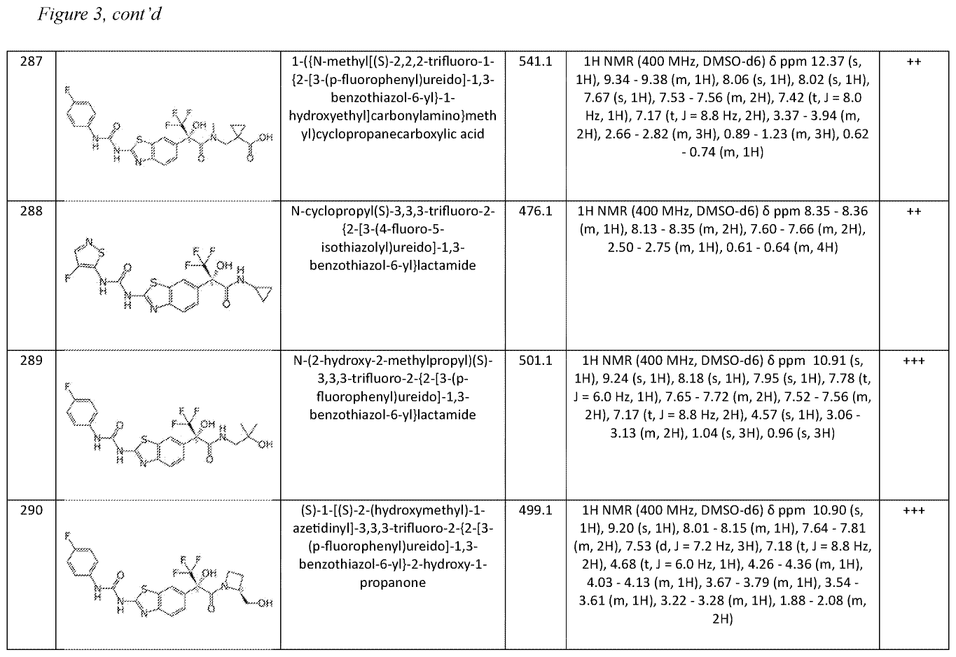

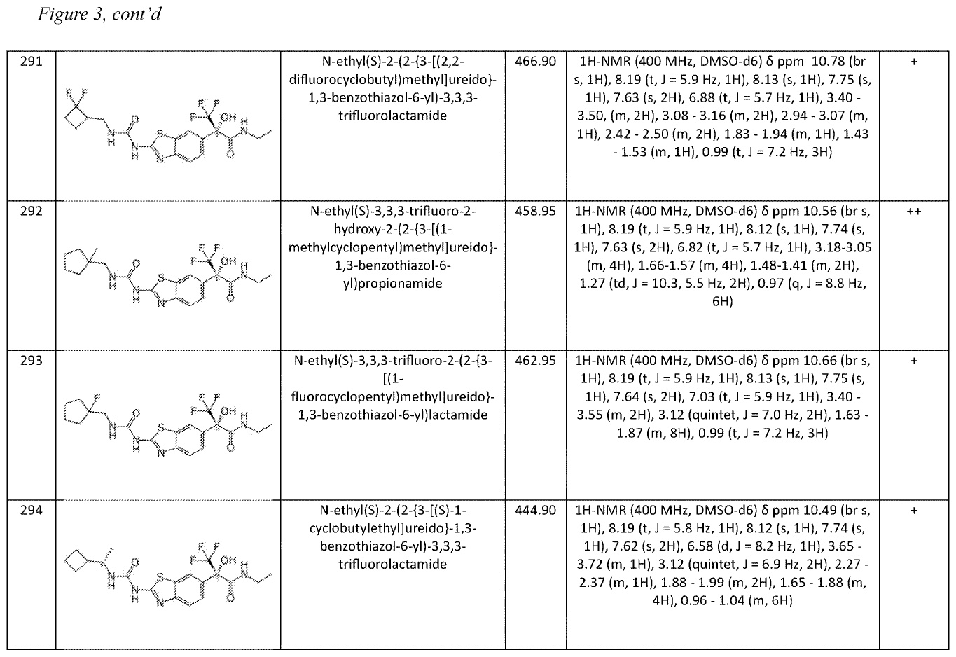

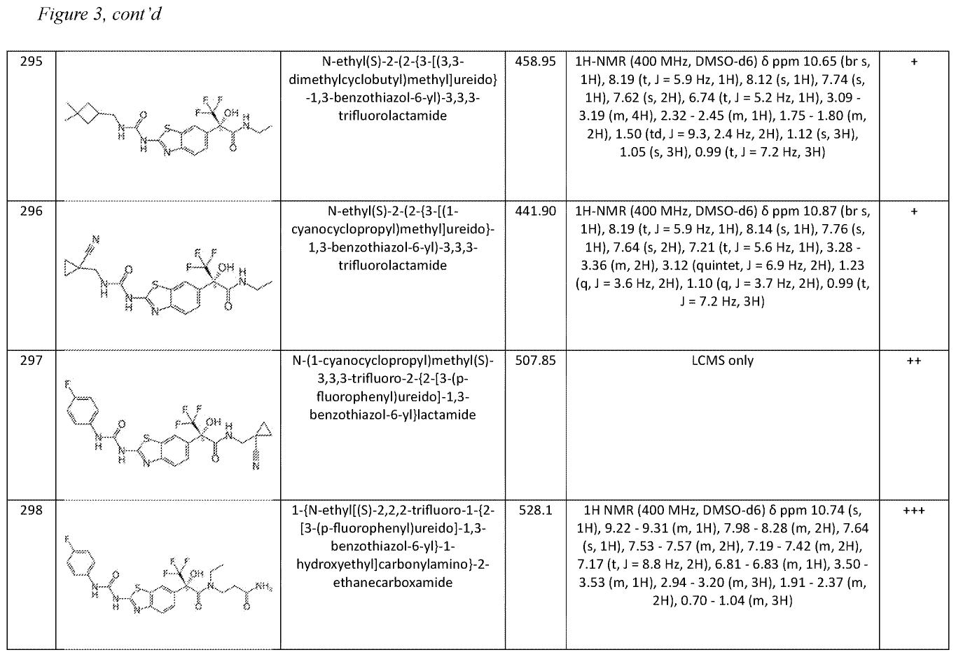

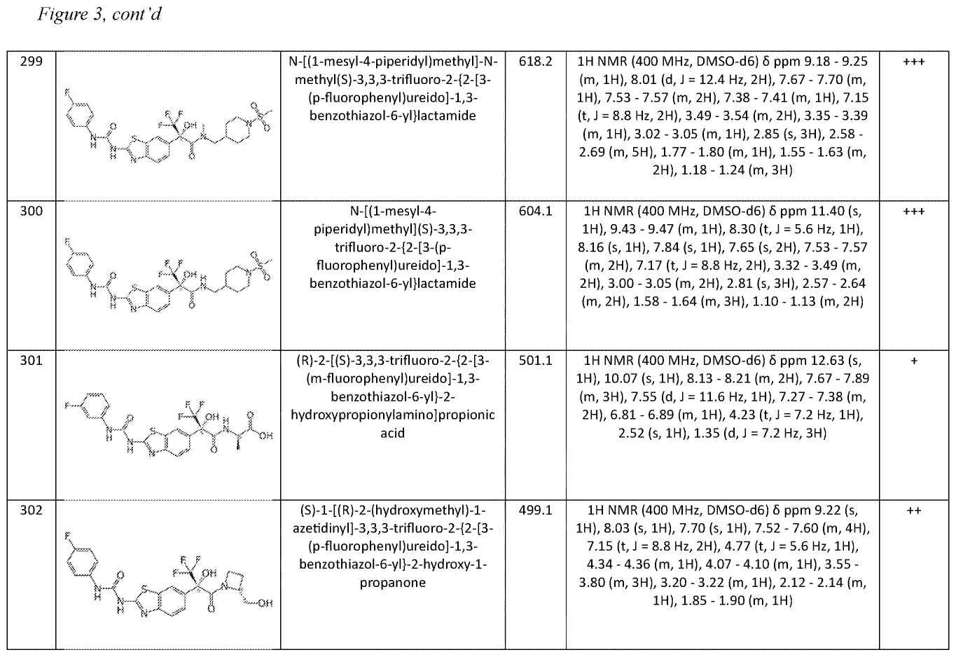

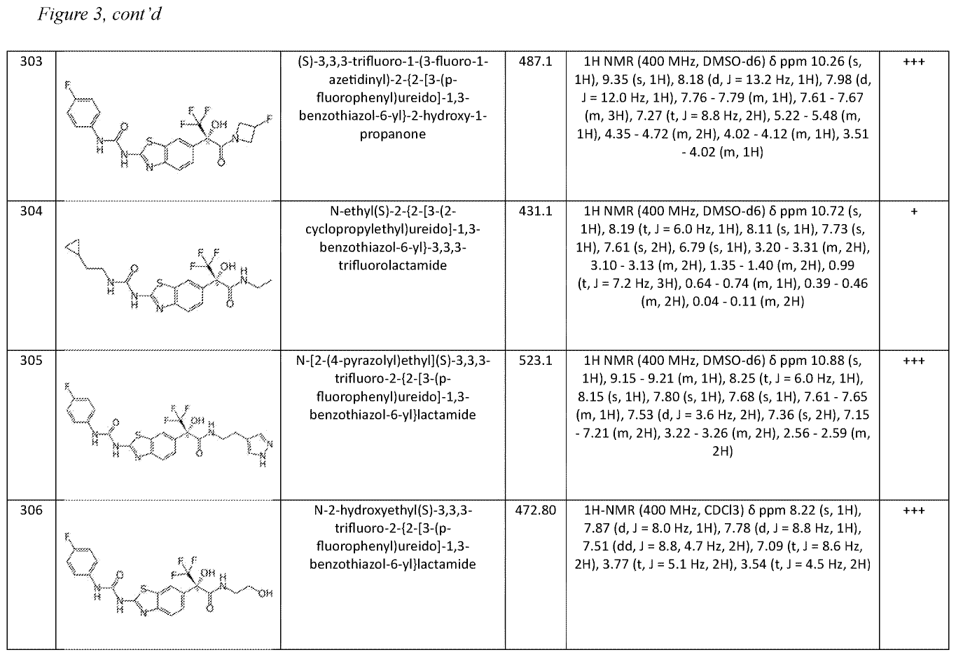

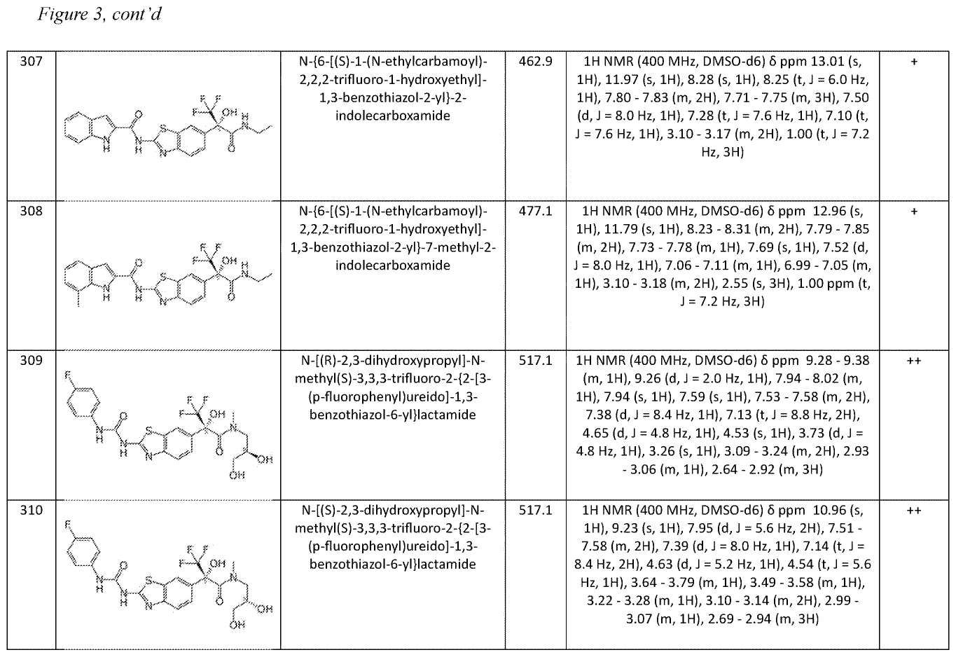

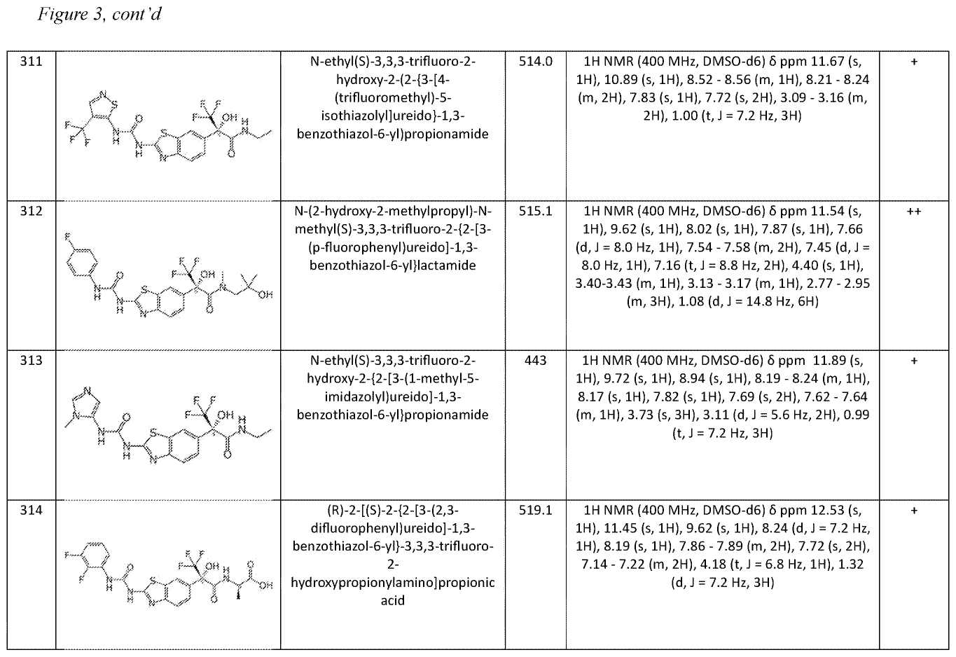

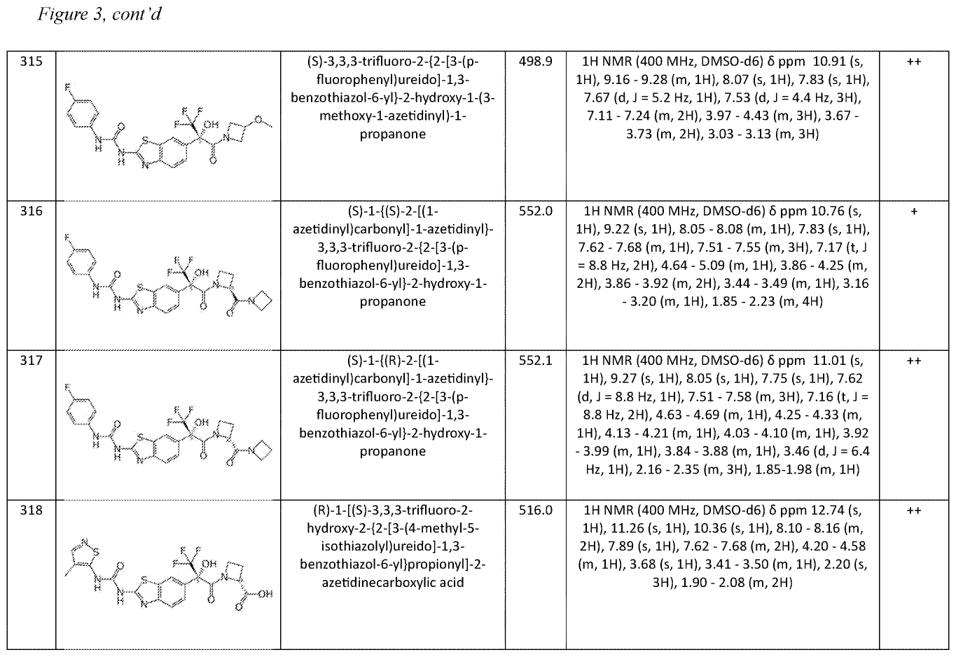

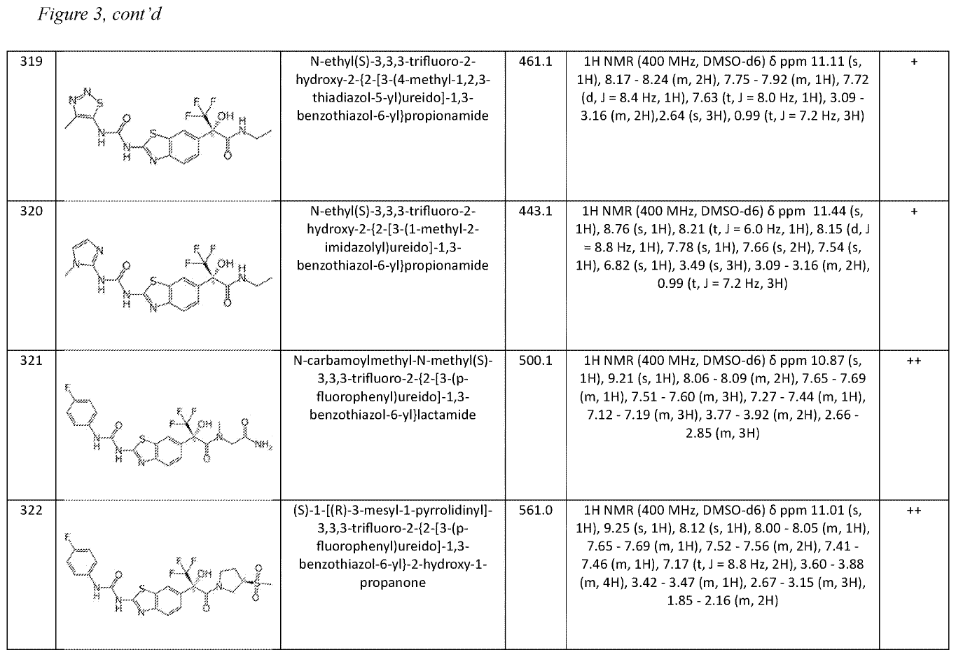

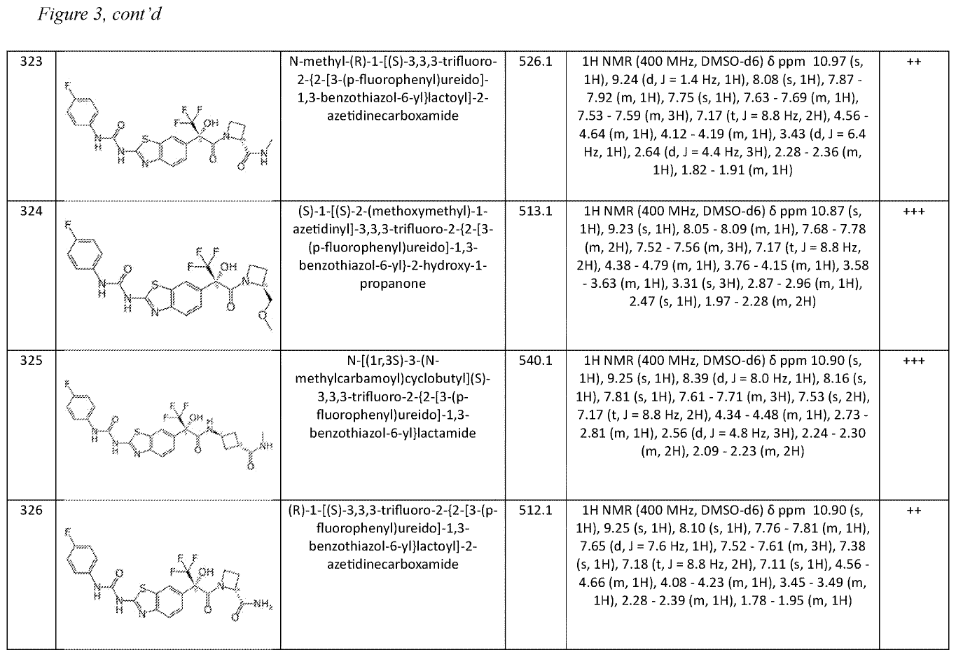

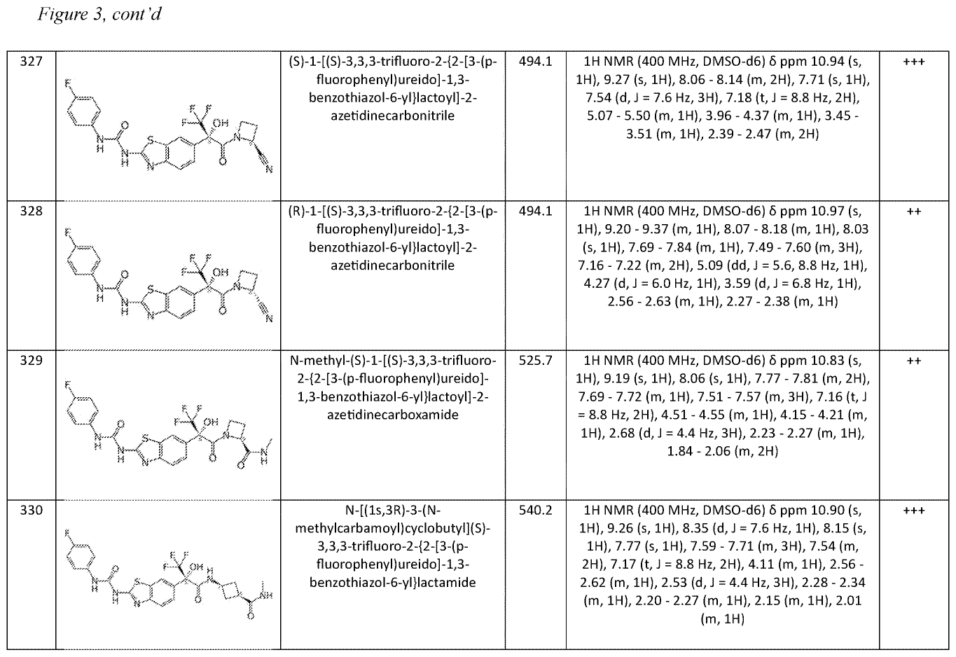

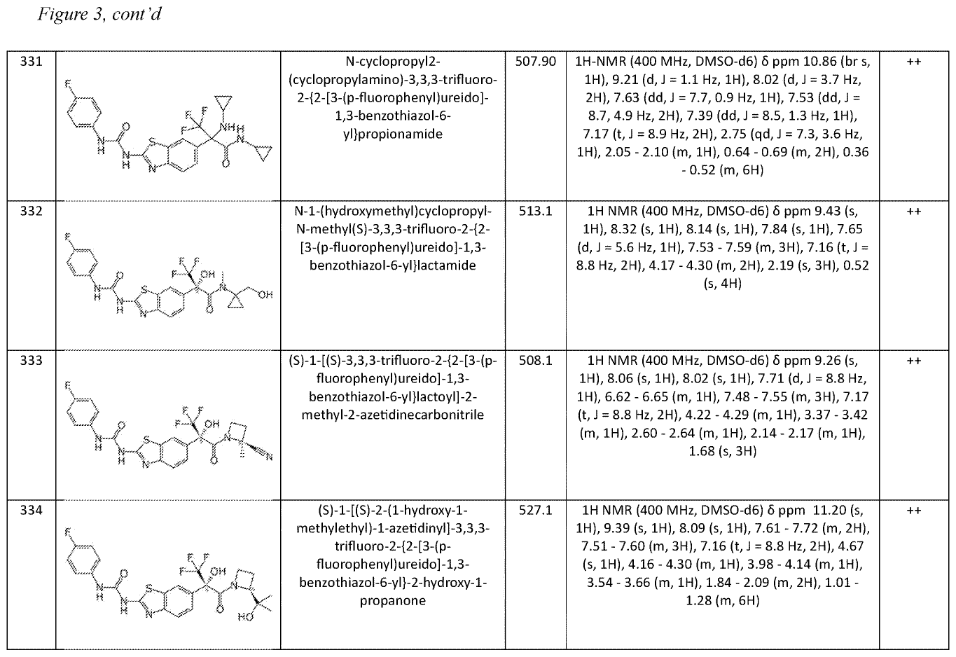

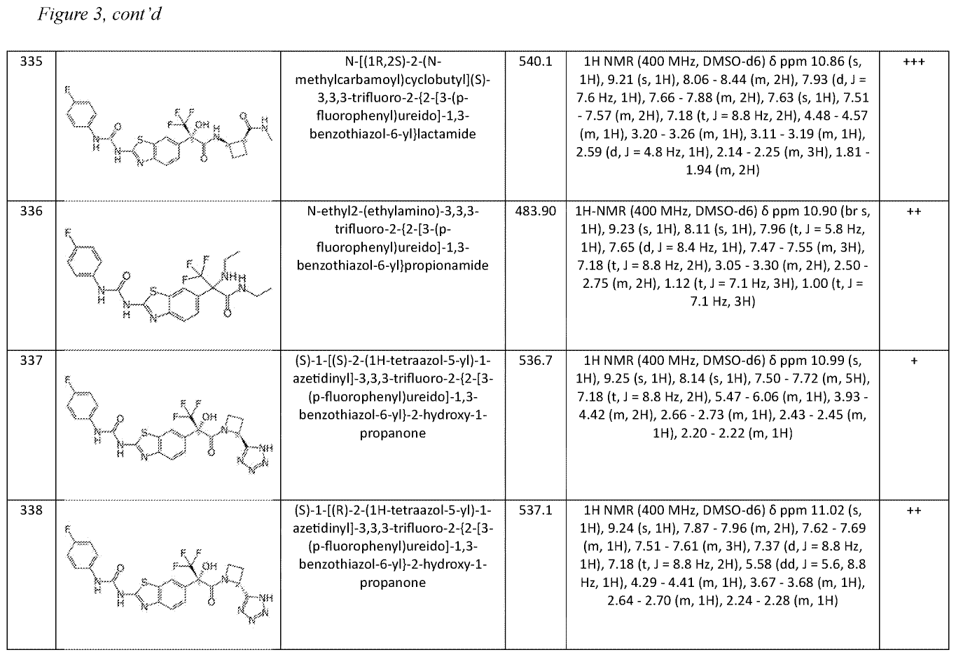

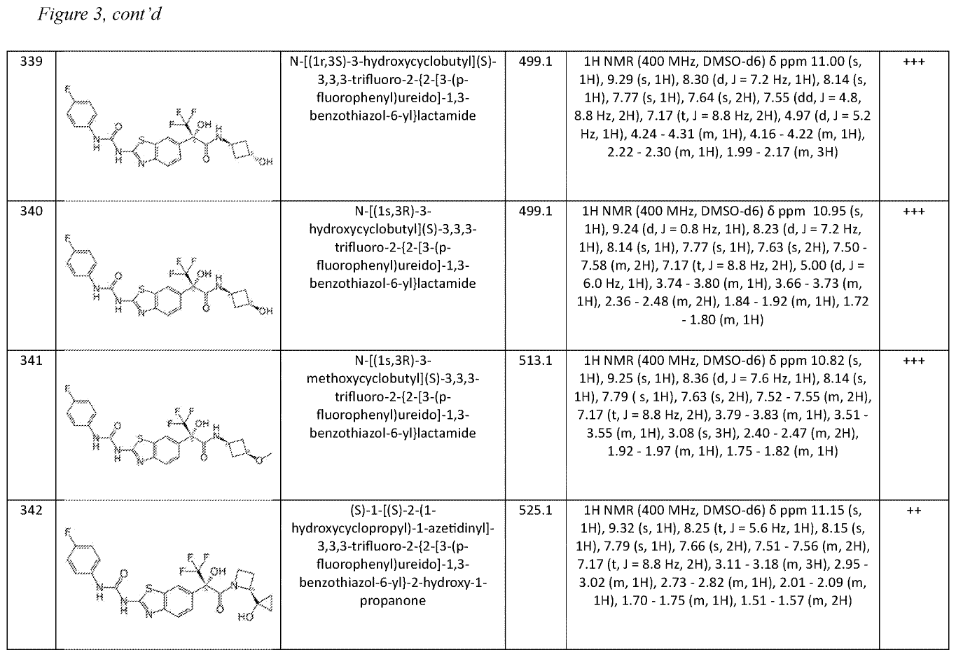

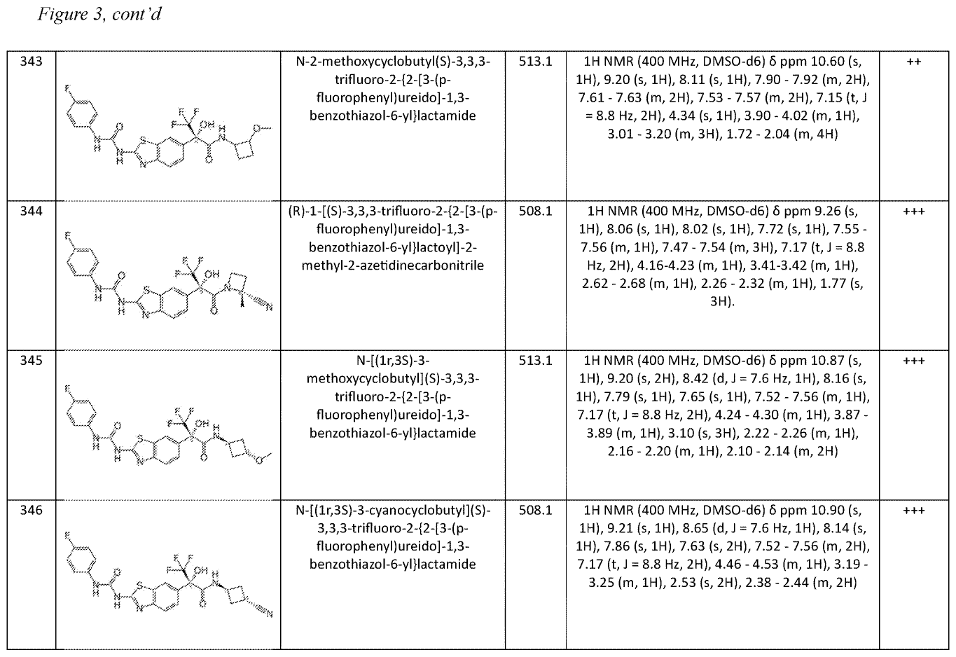

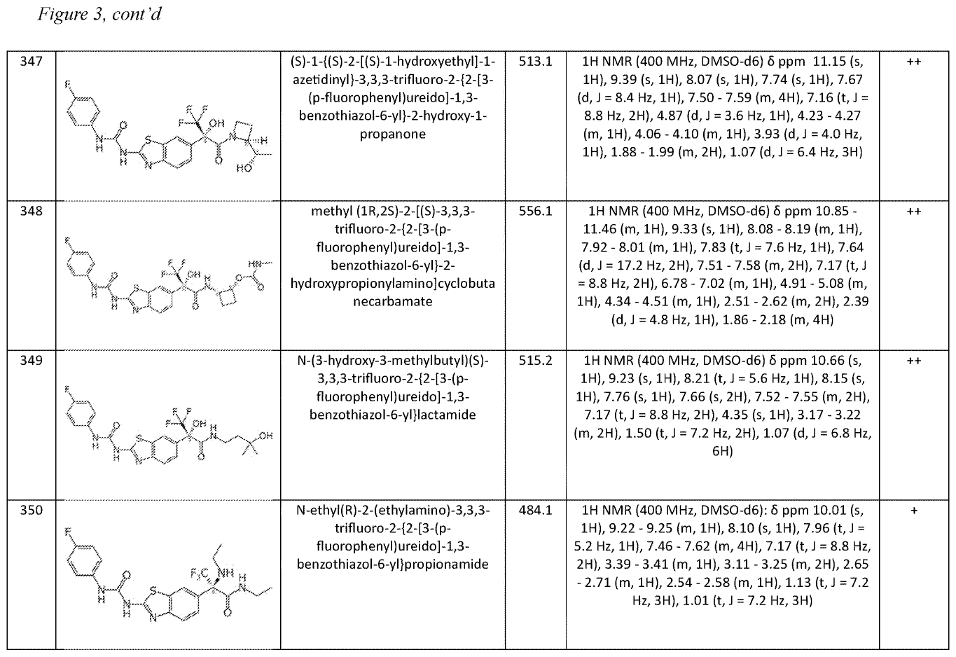

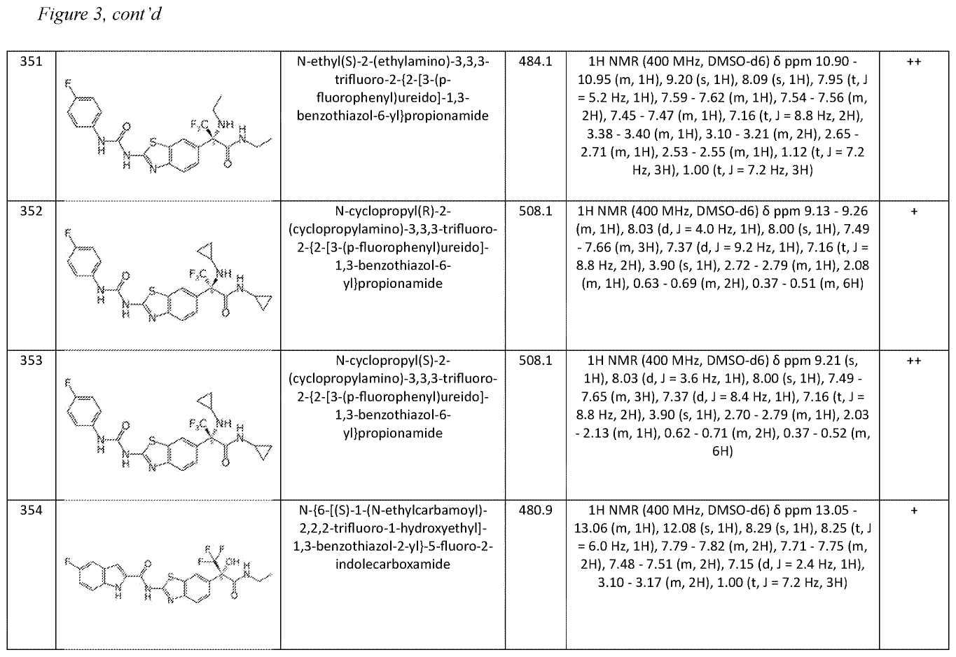

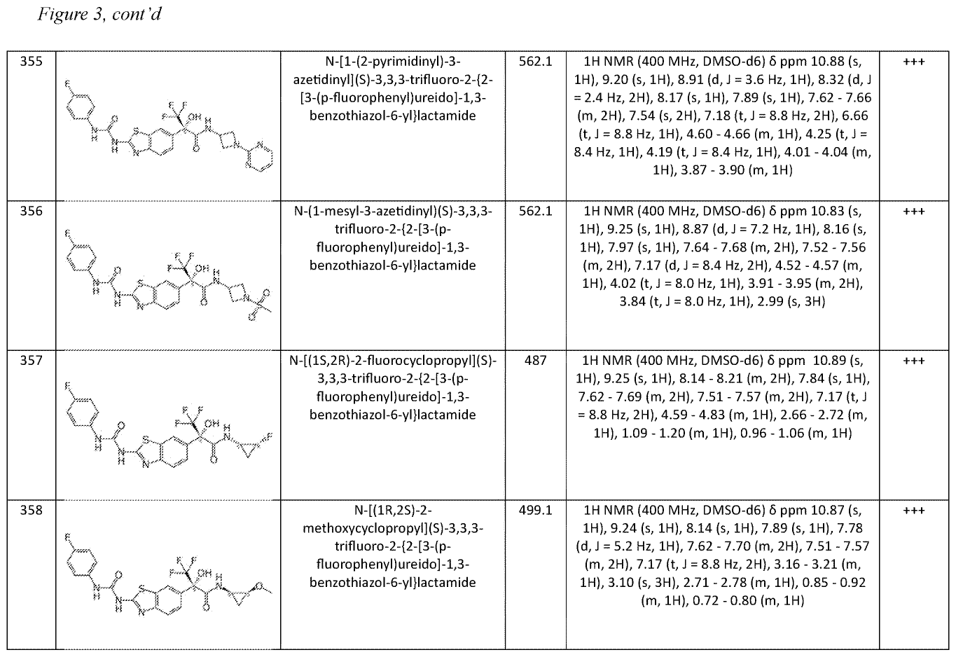

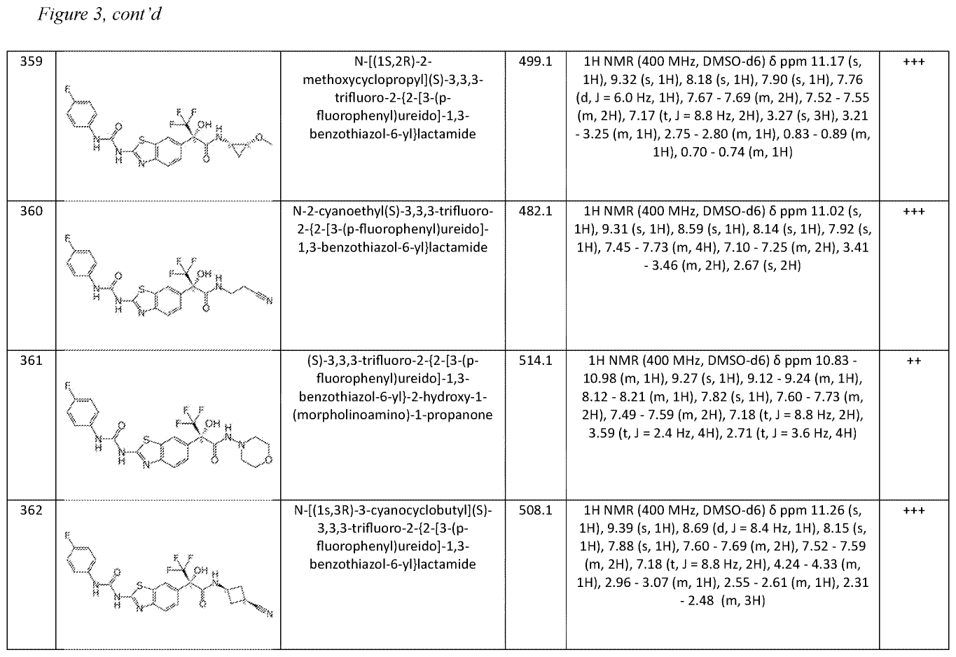

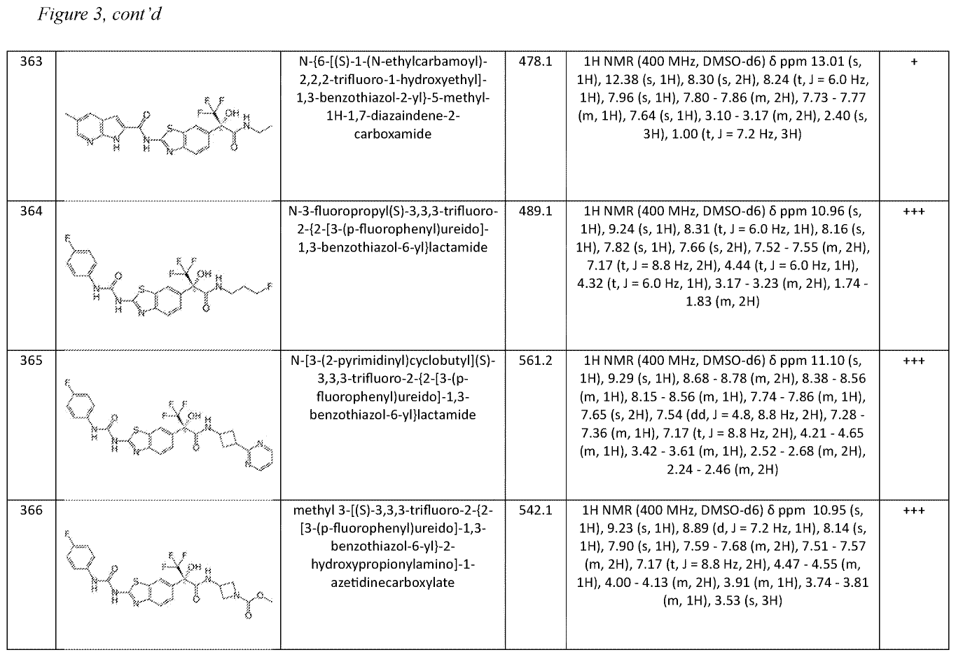

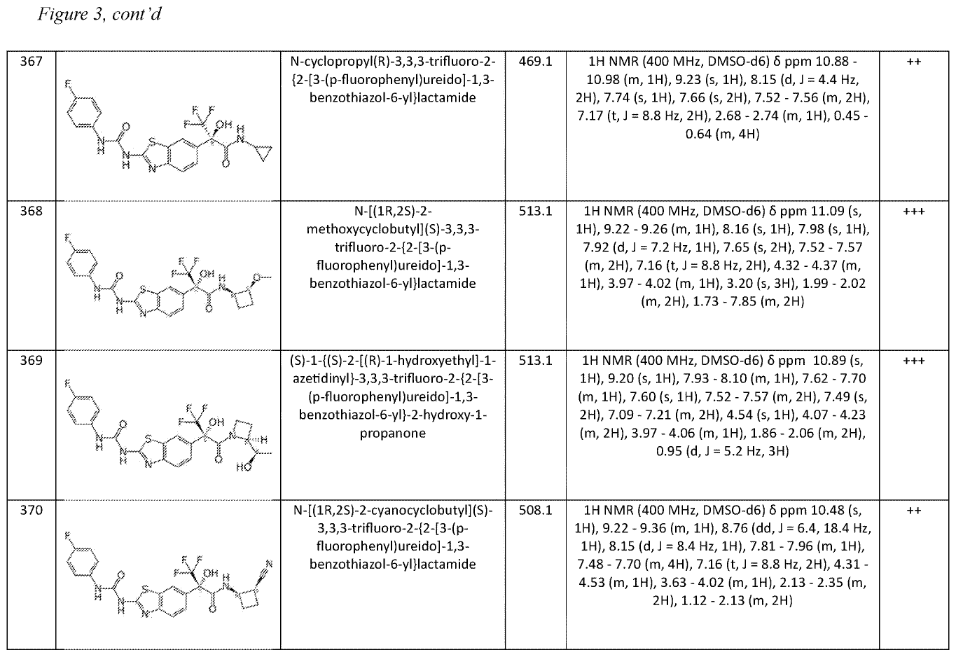

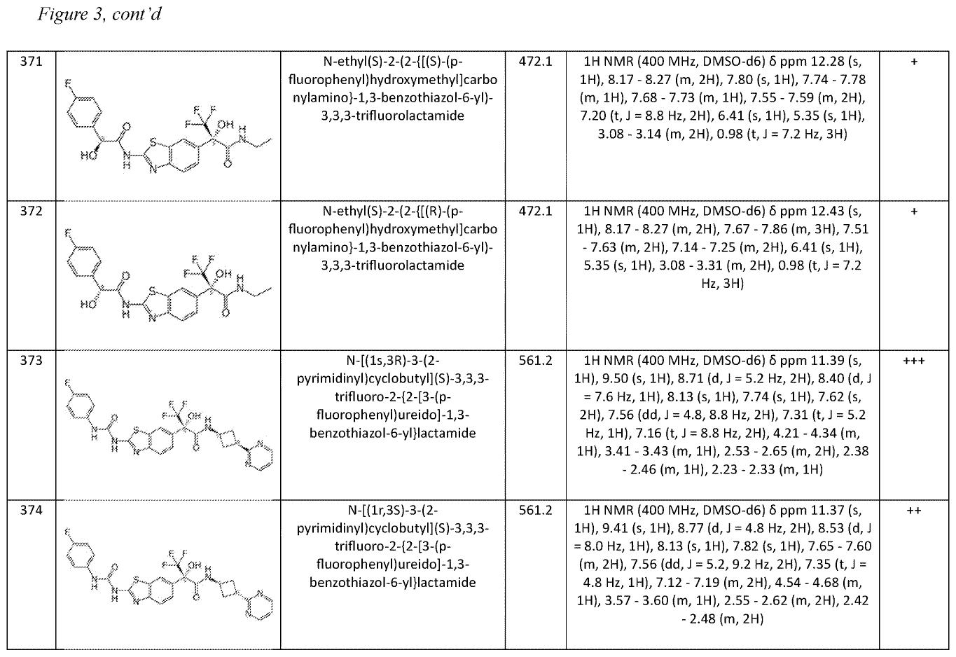

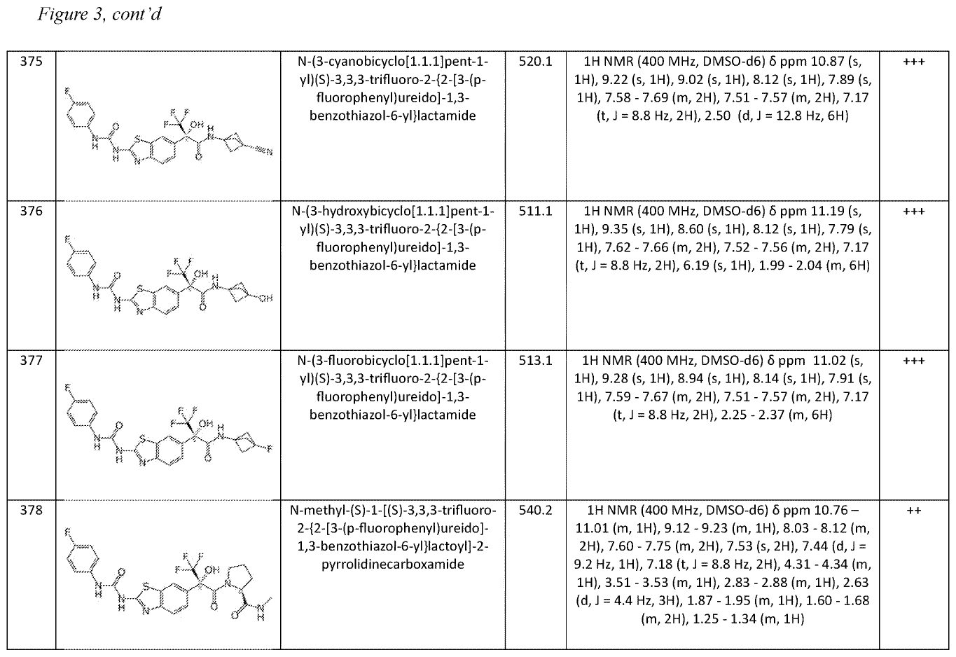

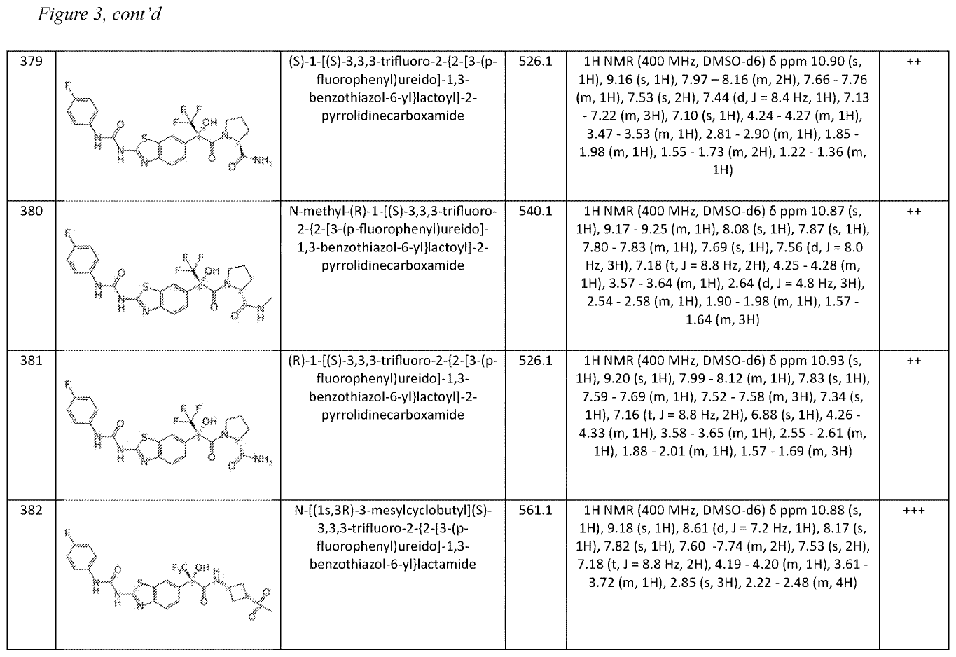

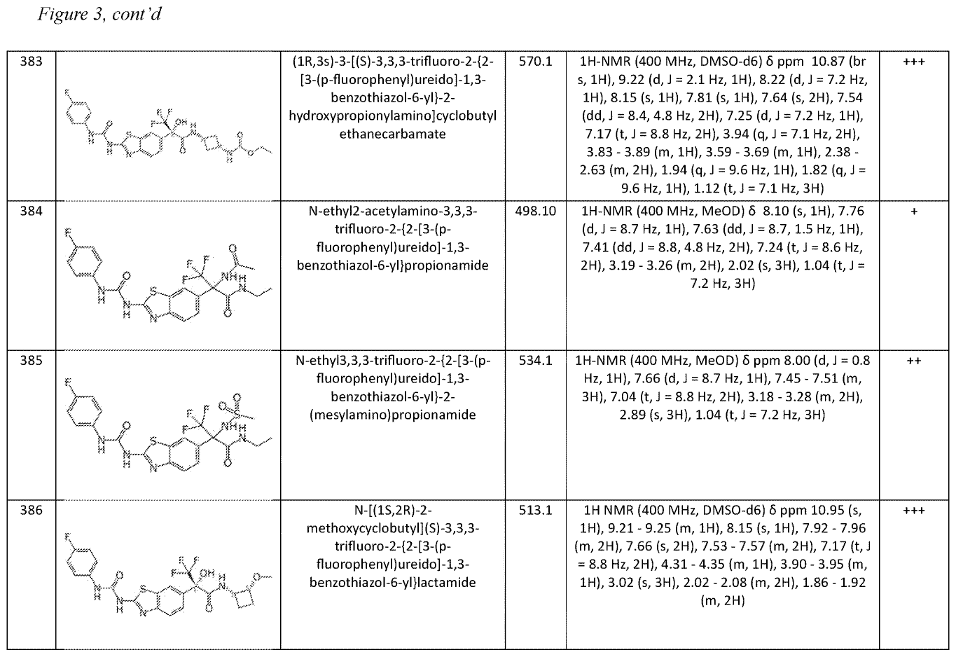

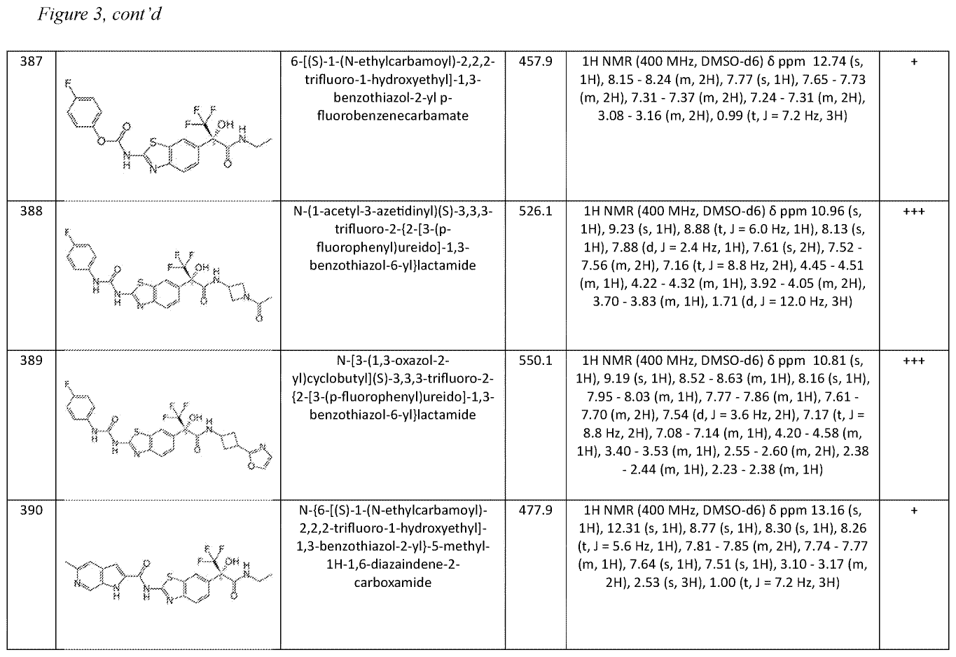

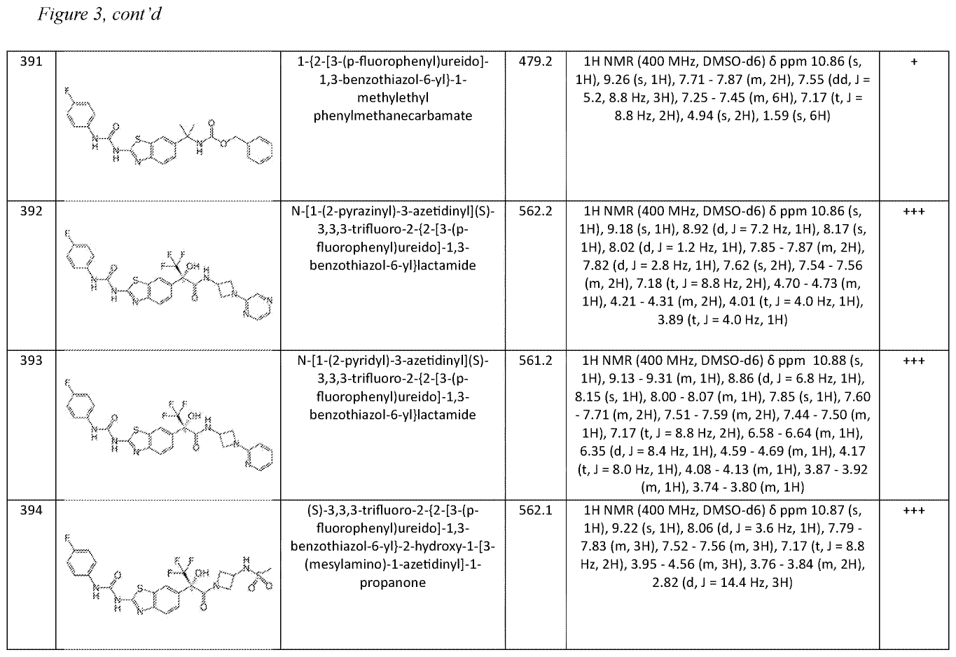

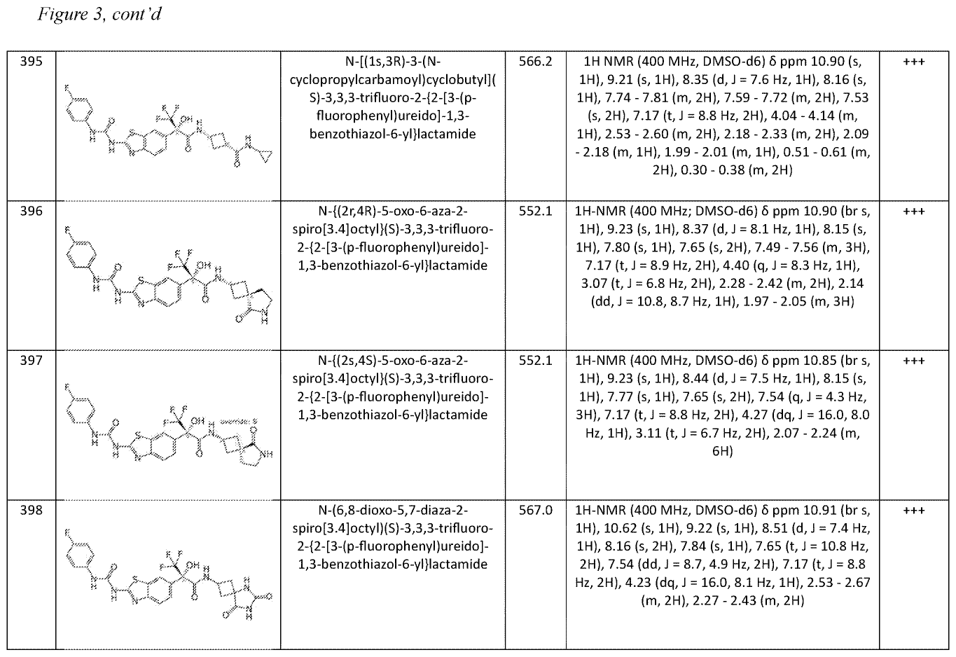

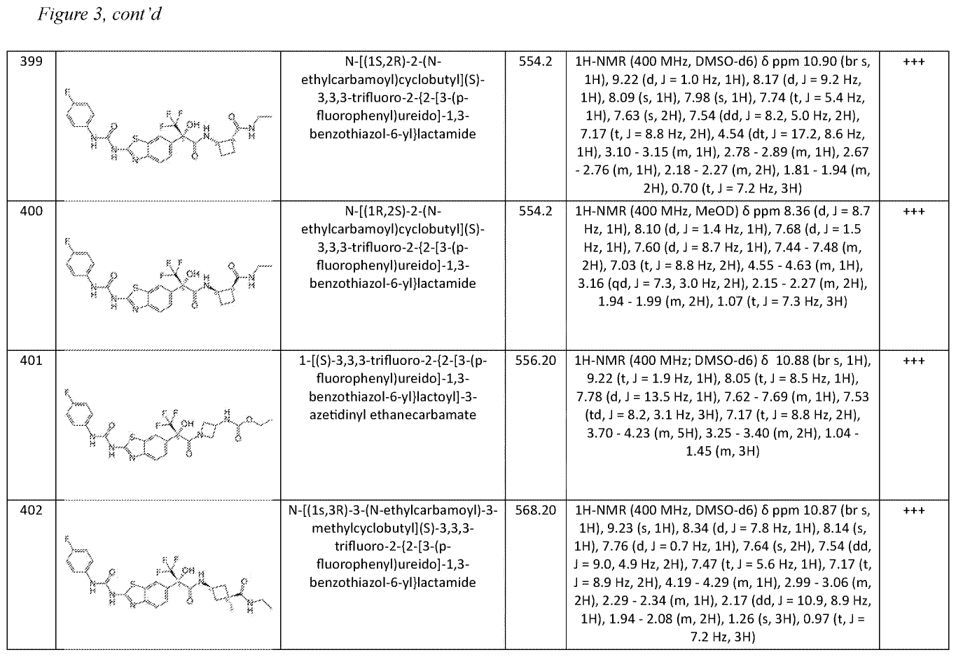

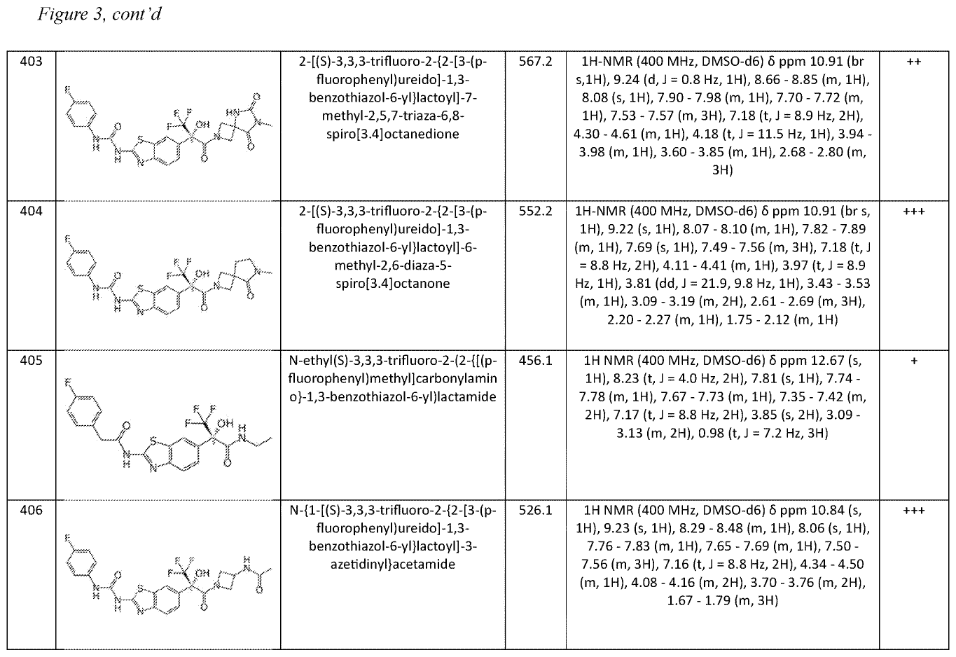

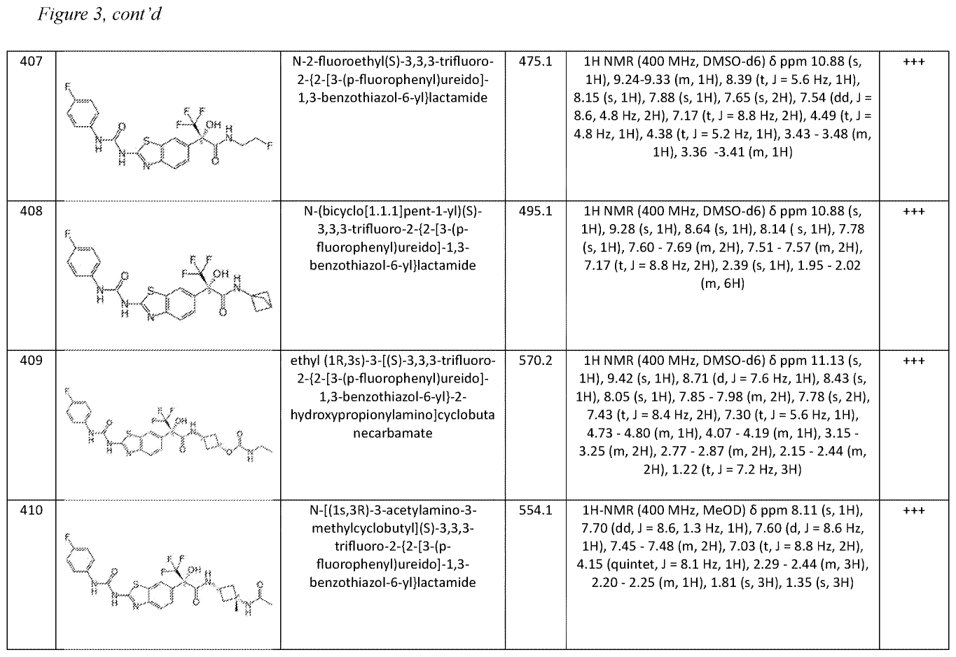

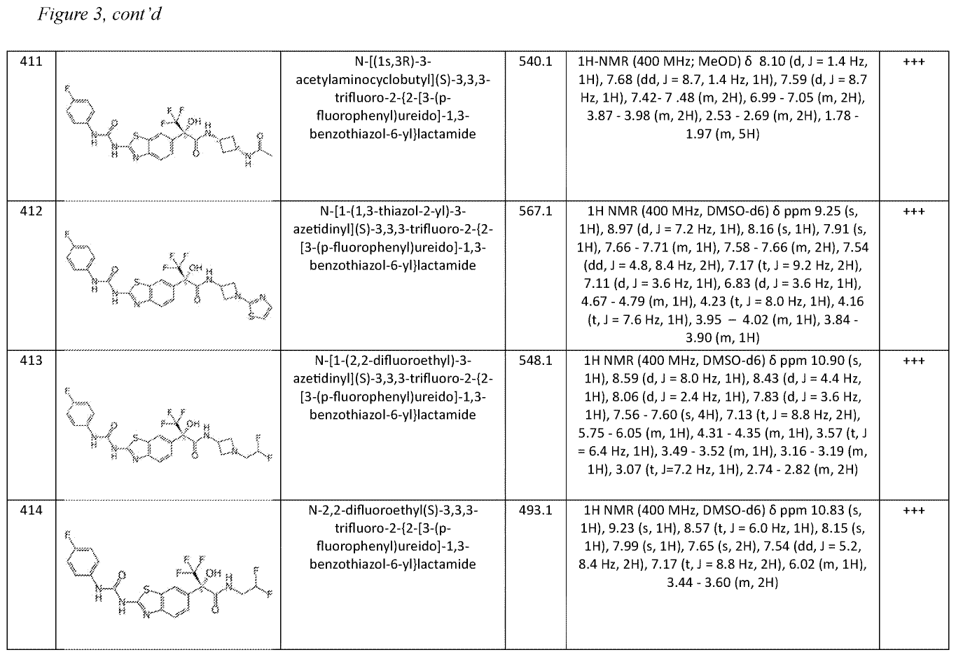

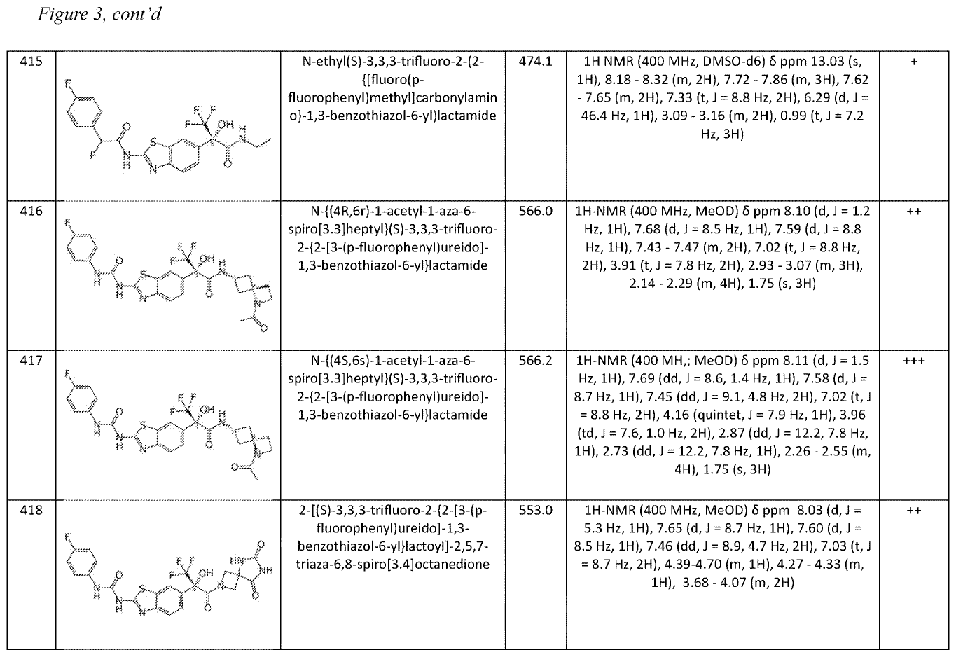

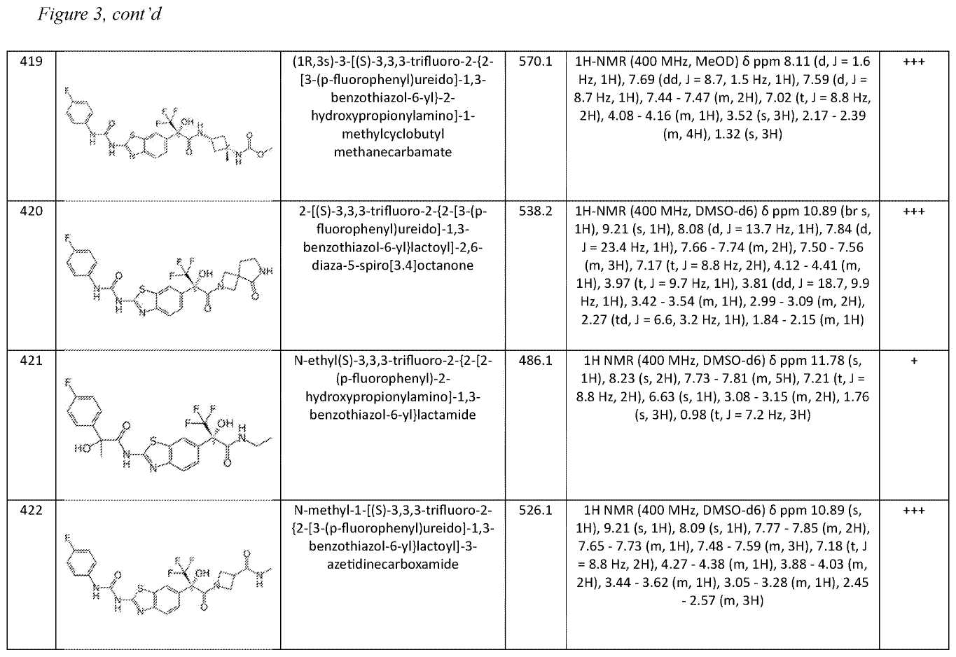

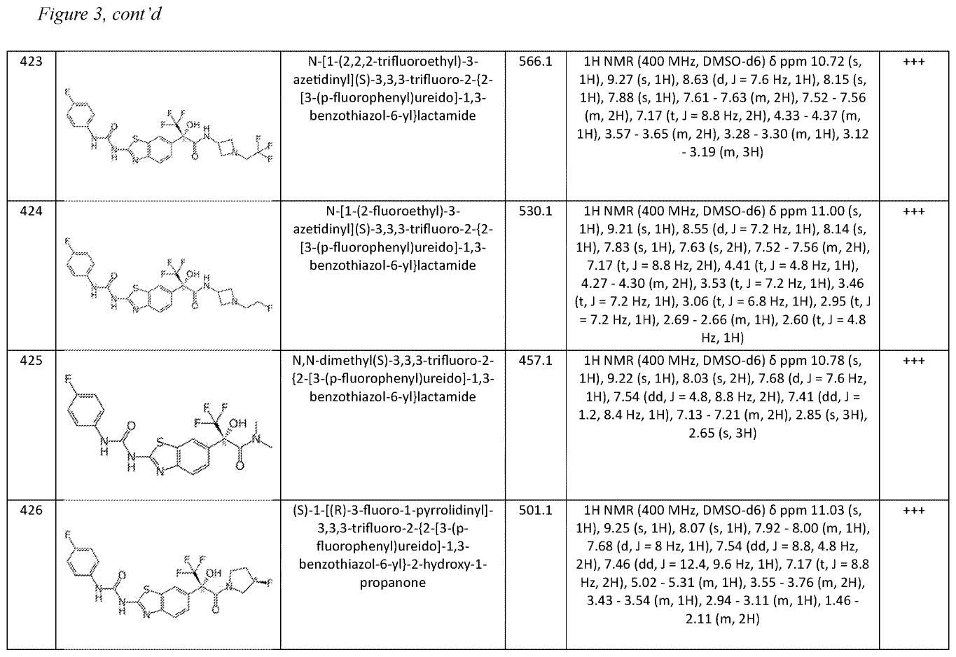

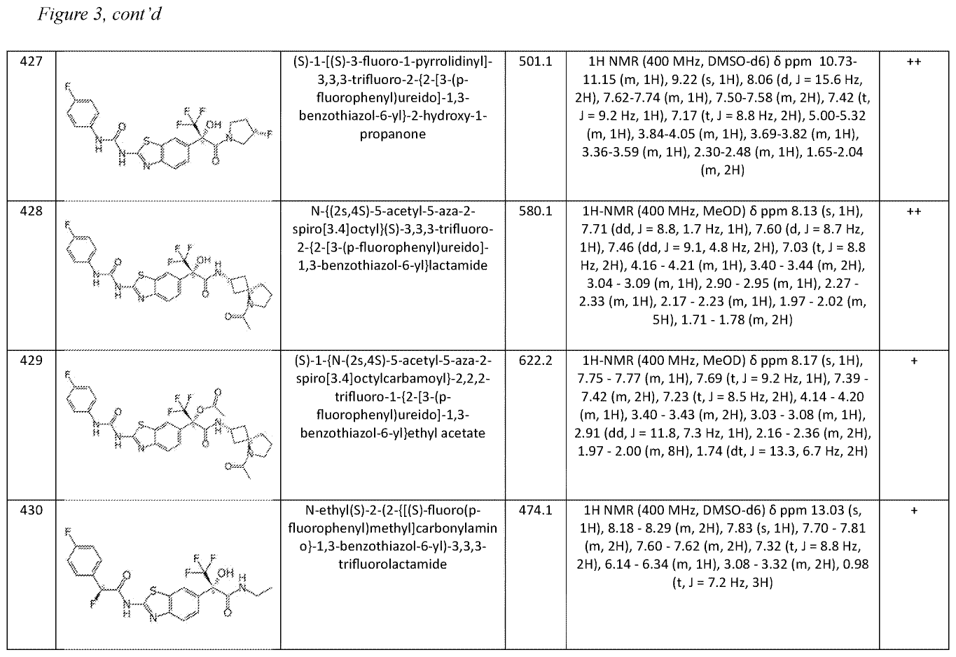

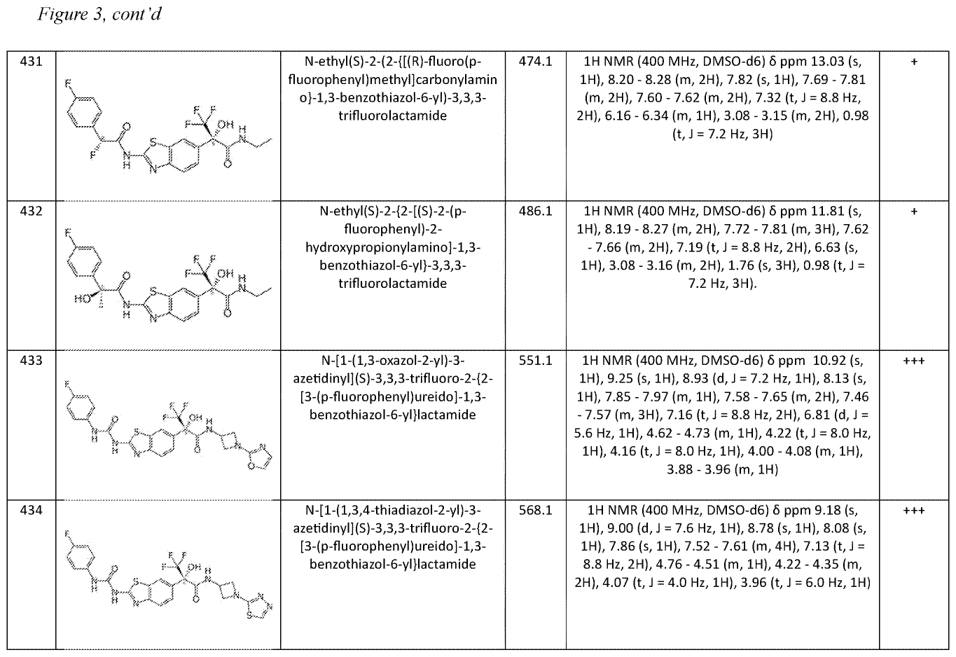

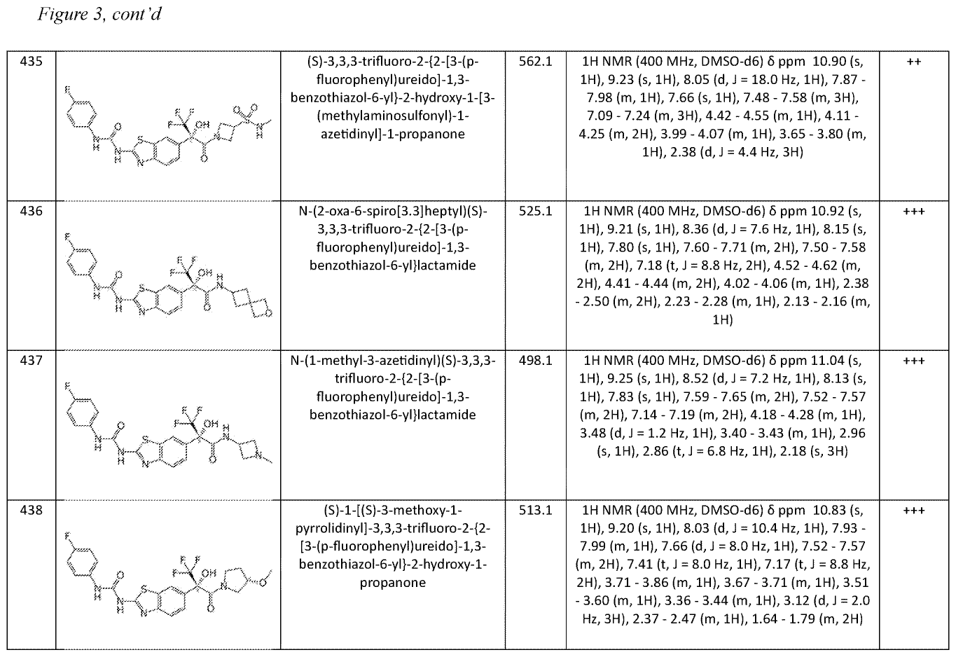

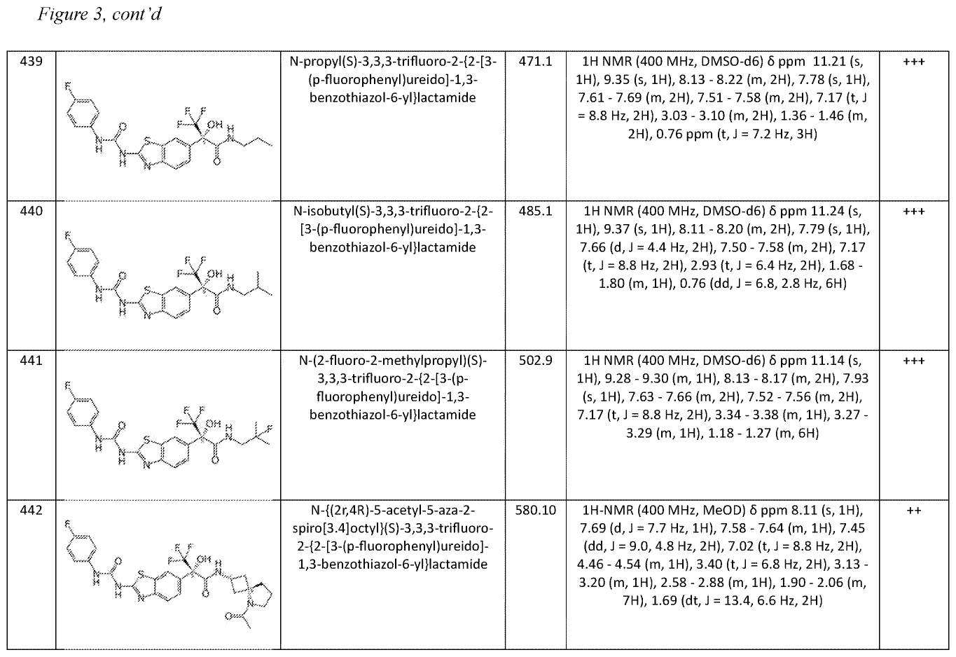

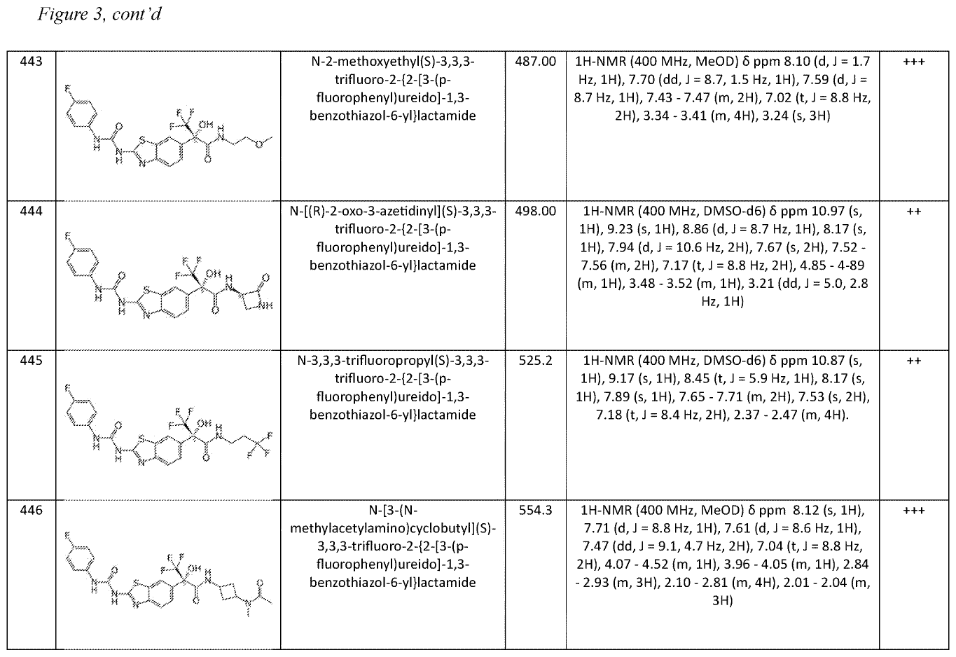

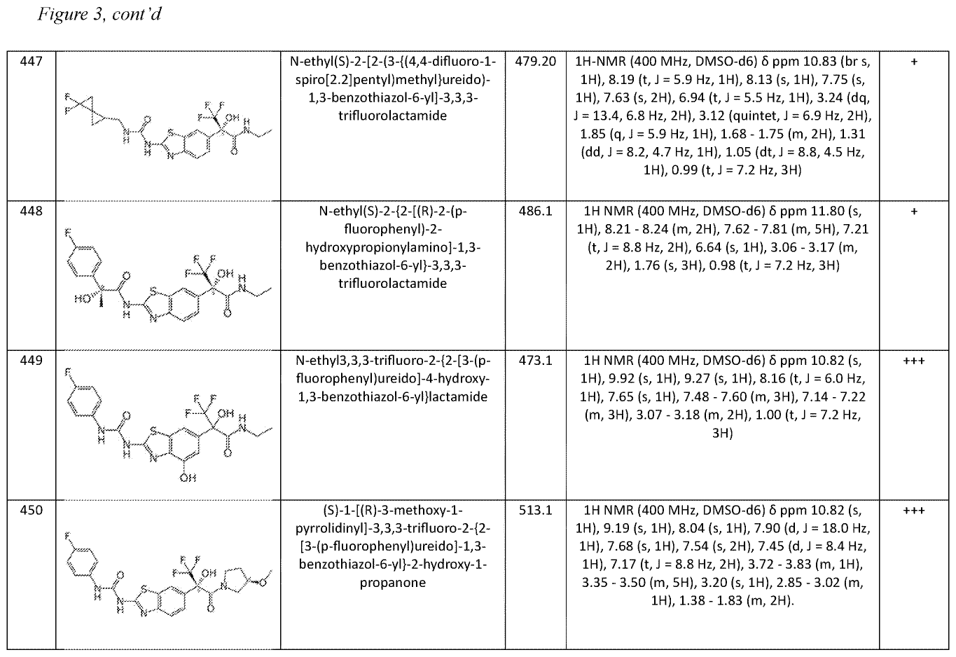

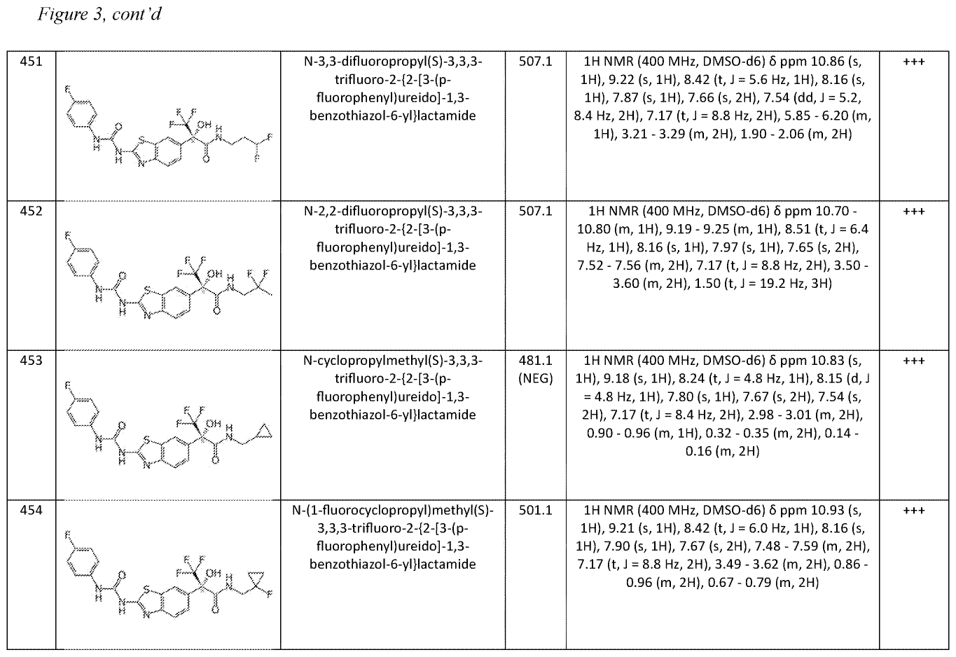

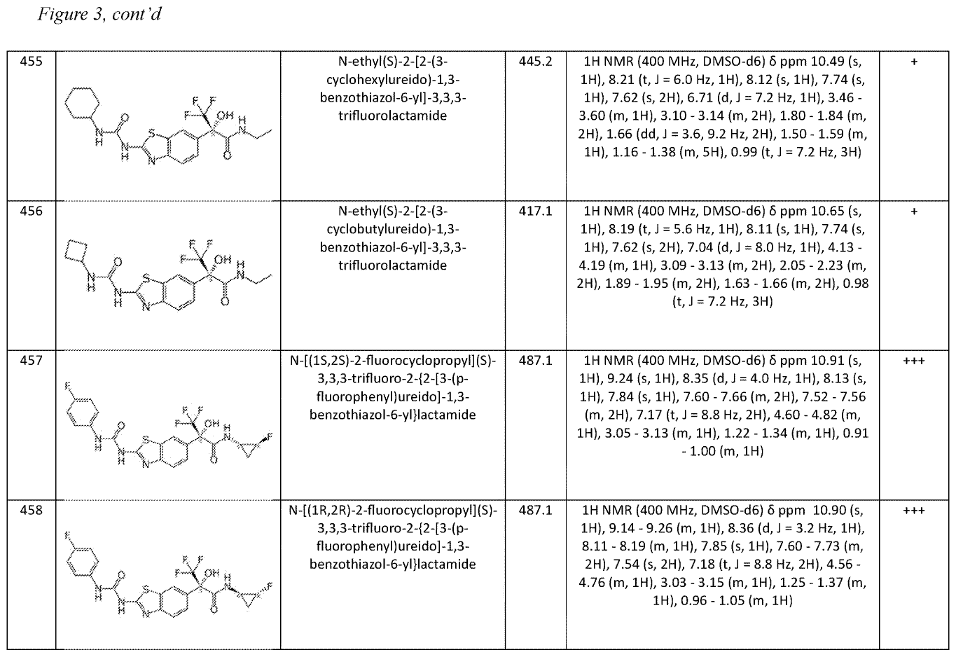

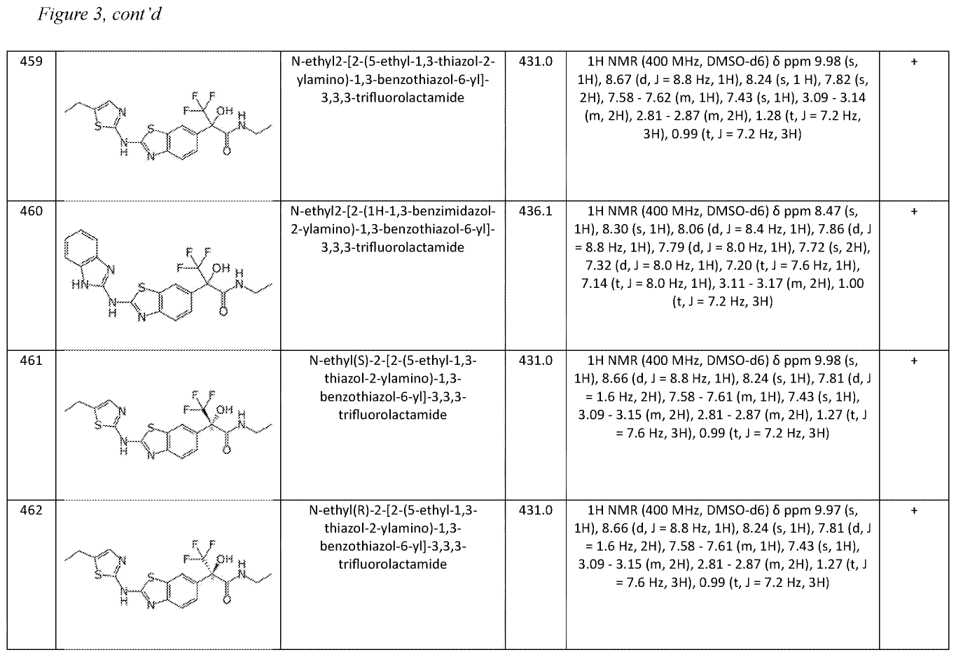

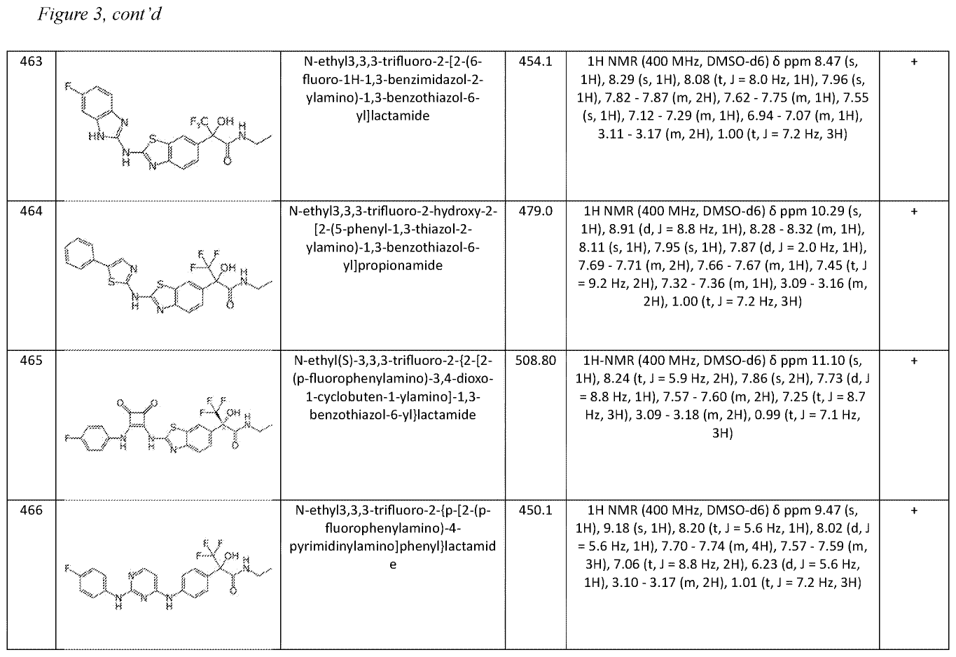

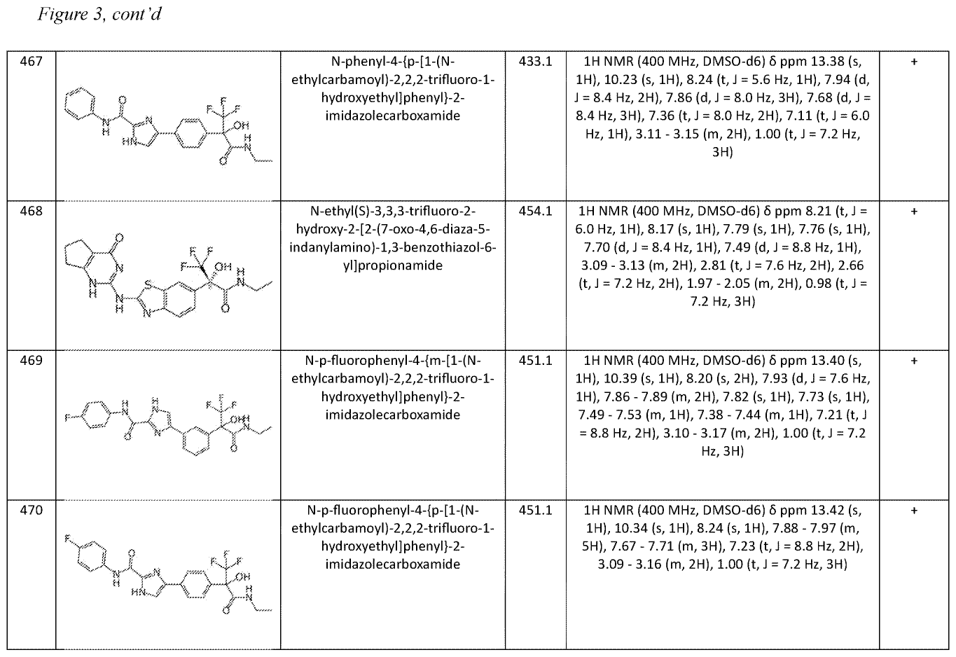

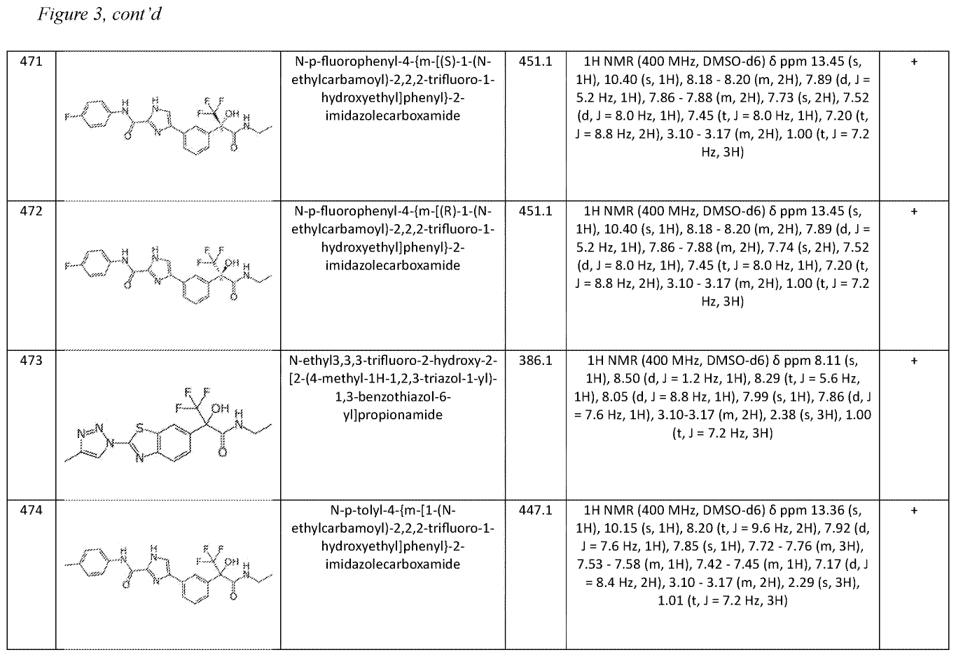

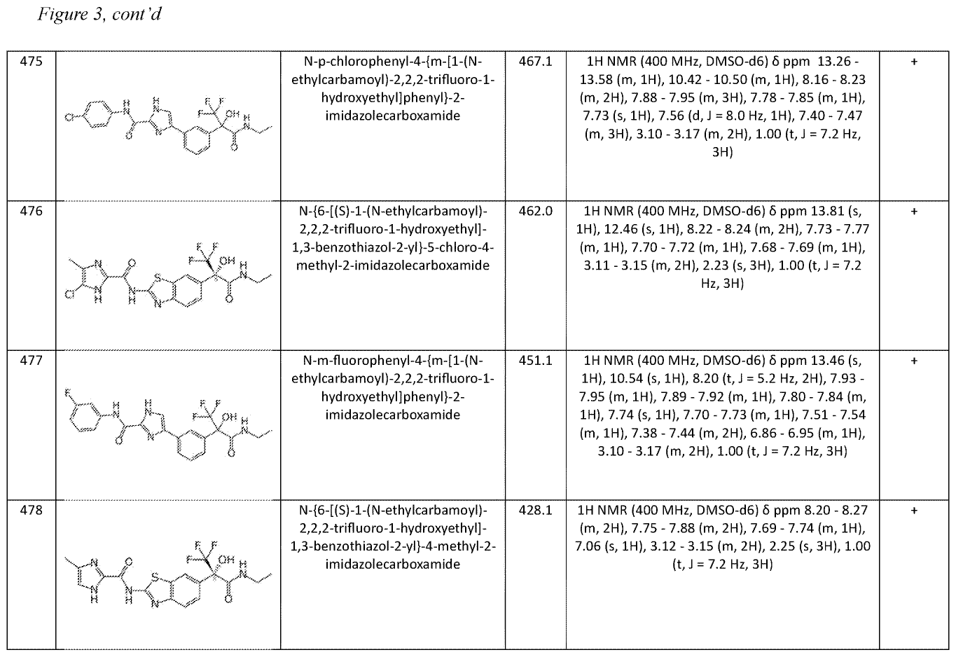

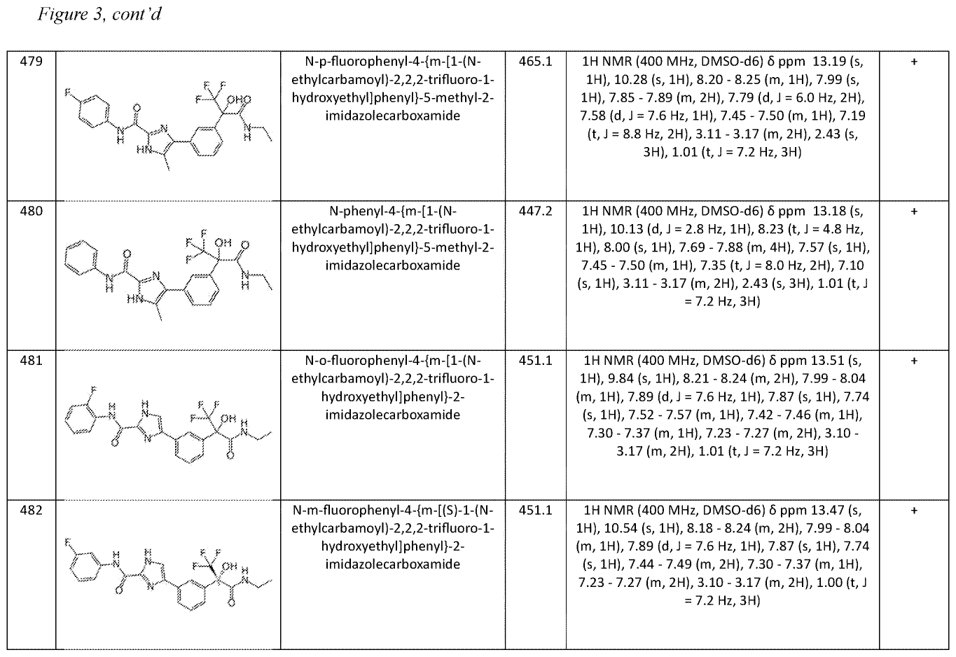

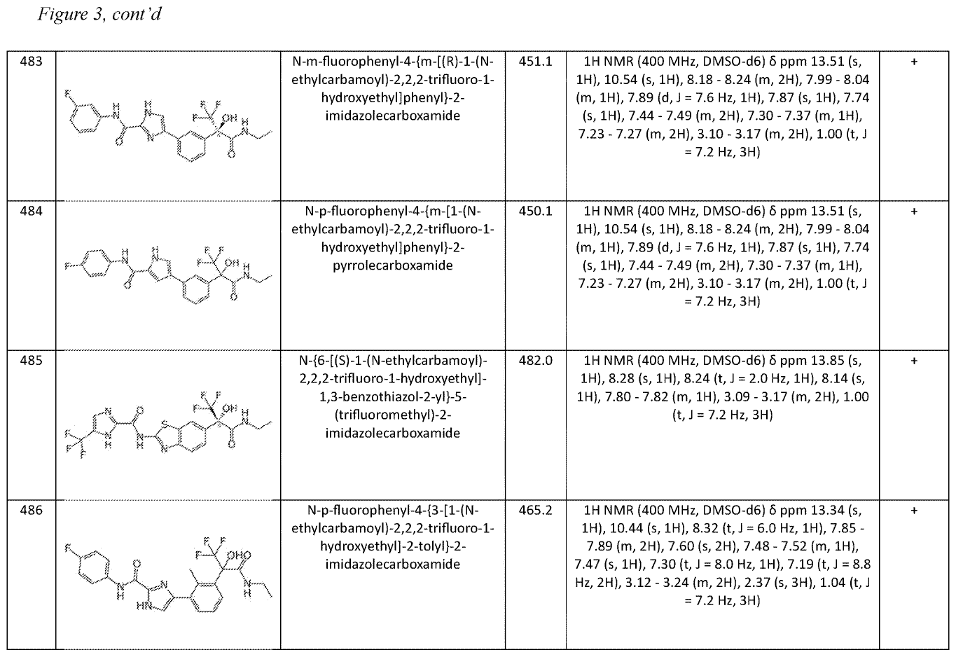

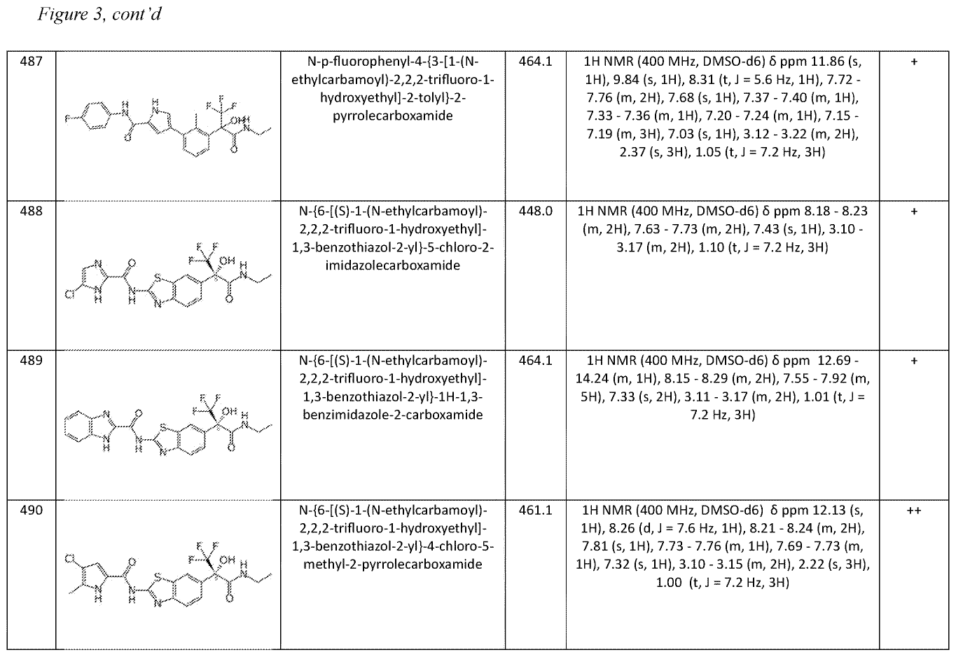

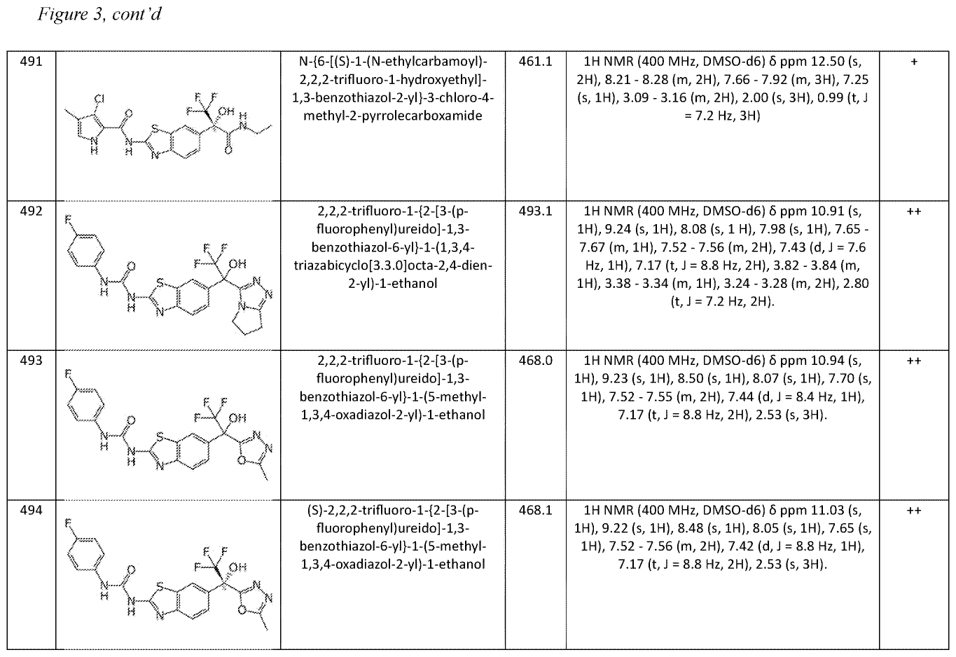

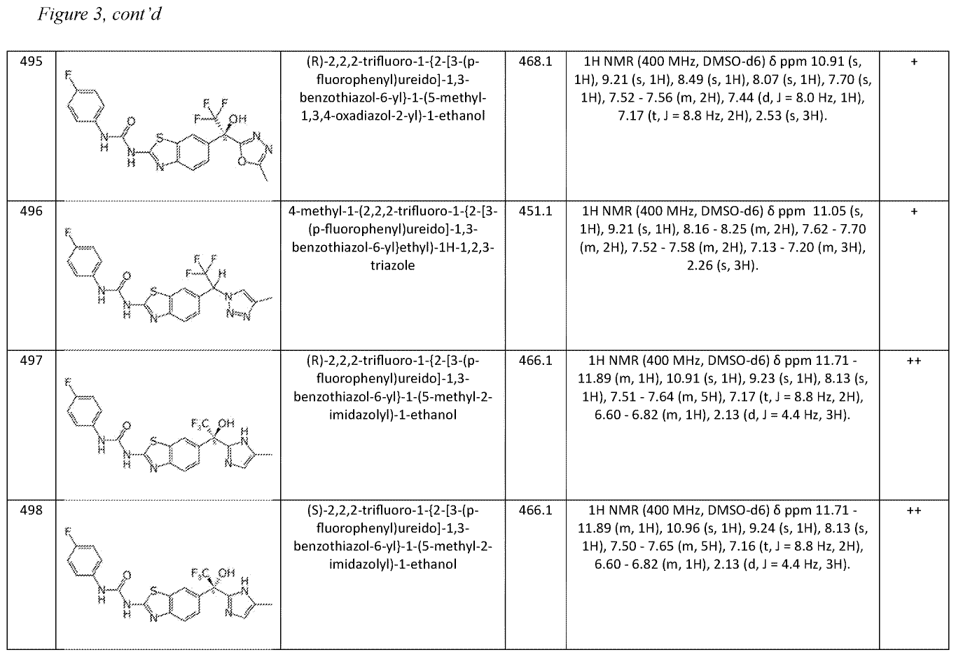

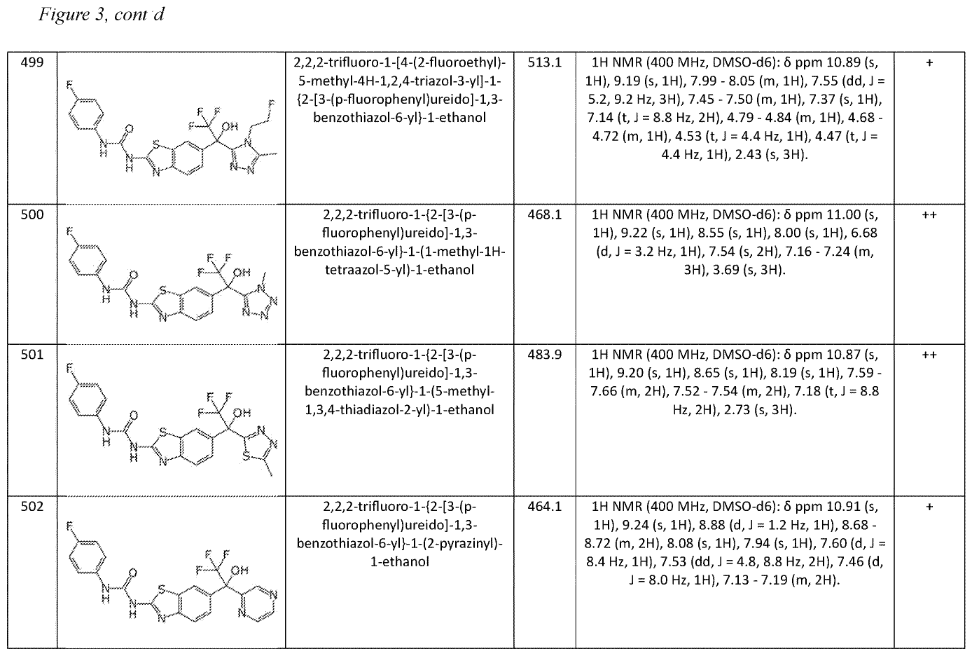

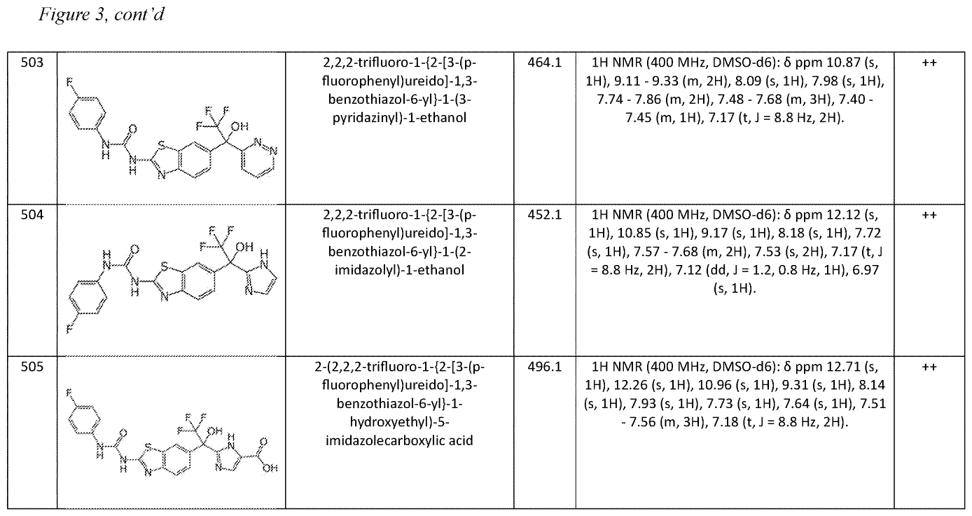

A complete listing of the compounds, characterization data, and assay data are set forth in the Tables in FIGS. 1, 2, and 3. The pEC₅₀ activity bins are: pBC₅₀>7: +++; pEC₅₀ between 6 and 7: ++; and pBC₅₀<6: +.

### INCORPORATION BY REFERENCE

All of the U.S. patents and U.S. and PCT patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A compound of Formula (I): 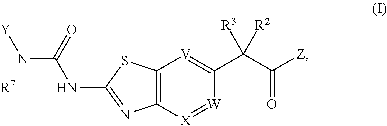
or a pharmaceutically acceptable salt thereof;
wherein:
Q is O or S;
V, W, and X are independently N or CR¹; provided that at least one of V, W, and X is CR¹;
Y is (C₁-C₆)alkyl, (C₃-C₈)cycloalkyl, 4- to 7-membered heterocycloalkyl, phenyl, or 5- or 6-membered heteroaryl; each of which is optionally substituted with one, two, or three substituents selected independently for each occurrence from fluoro, chloro, cyano, hydroxyl, (C₁-C₆)alkyl, (C₃-C₈)cycloalkyl, (C₁-C₆)haloalkyl, (C₁-C₆)hydroxyalkyl, (C₁-C₆)alkoxyalkyl, and phenyl: each of which substituents is optionally substituted with one, two, or three substituents selected independently for each occurrence from fluoro, (C₁-C₆)alkyl, (C₁-C₆)haloalkyl, (C₁-C₆)hydroxyalkyl, and (C₁-C₆)alkoxyalkyl;
Z is NR⁴R⁵ or OR⁶;
R¹ is independently for each occurrence H, (C₁-C₆)alkyl, (C₁-C₆)haloalkyl, (C₁-C₆)hydroxyalkyl, (C₁-C₆)alkoxyalkyl, fluoro, chloro, cyano, hydroxyl, or (C₁-C₆)alkoxy;
R² is (C₁-C₆)haloalkyl, (C₁-C₆)alkyl, or (C₃-C₈)cycloalkyl;
R³ is hydroxyl, (C₁-C₆)alkyl, (C₁-C₆)alkoxyalkyl, (C₁-C₆)hydroxyalkyl, NR¹¹R¹², NC(O)R¹³, OC(O)NR¹¹R¹², or NR¹⁴SO₂R¹⁵; or R² and R³ together with the carbon atom to which they are attached form a (C₃-C₈)cycloalkyl or a 4- to 7-membered heterocycloalkyl;
wherein (C₃-C₈)cycloalkyl or 4- to 7-membered heterocycloalkyl is optionally substituted with one, two, or three substituents selected independently for each occurrence from fluoro, chloro, cyano, hydroxyl, (C₁-C₆)alkyl, (C₁-C₆)haloalkyl, (C₁-C₆)hydroxyalkyl, and (C₁-C₆)alkoxyalkyl;
R⁴ is hydrogen, (C₁-C₆)alkyl, (C₃-C₈)cycloalkyl, 4- to 7-membered heterocycloalkyl, phenyl, or 5- or 6-membered heteroaryl; wherein (C₁-C₆)alkyl is optionally substituted with one, two or three substituents selected independently for each occurrence from R^{4a}, halo, hydroxyl, cyano, carboxy, (C₁-C₆)alkylsulfonyl, (C₁-C₆)alkylcarbonyl, C(O)NR¹¹R¹², NR¹¹R¹², OC(O)NR¹¹R¹² and (C₁-C₆)alkoxy; and wherein (C₃-C₈)cycloalkyl, 4- to 7-membered heterocycloalkyl, phenyl, or 5- or 6-membered heteroaryl is optionally substituted with one, two, or three substituents selected independently for each occurrence from fluoro, chloro, cyano, hydroxyl, 5- to 10-membered heteroaryl, (C₁-C₆)alkyl, (C₁-C₆)haloalkyl, (C₁-C₆)hydroxyalkyl, (C₁-C₆)alkoxyalkyl, (C₁-C₆)alkylsulfonyl, (C₁-C₆)alkylcarbonyl C(O)NR¹¹R¹², NR¹¹R¹², NC(O)OR¹³, NC(O)R¹³, OC(O)NR¹¹R¹², and SO₂NR¹¹R¹²;
R^{4a} is (C₃-C₈)cycloalkyl, phenyl, 5- to 10-membered heteroaryl, or 4- to 7-membered heterocycloalkyl, each of which is optionally substituted with one, two, or three substituents selected independently for each occurrence from fluoro, chloro, cyano, hydroxyl, (C₁-C₆)alkyl, (C₁-C₆)haloalkyl, (C₁-C₆)hydroxyalkyl, (C₁-C₆)alkoxyalkyl, and (C₁-C₆)alkylsulfonyl;
R⁵ is hydrogen or (C₁-C₆)alkyl; or R⁴ and R⁵ taken together with the nitrogen atom to which they are attached form an optionally 4- to 7-membered heterocycloalkyl; wherein 4- to 7-membered heterocycloalkyl is optionally substituted with one, two, or three substituents selected independently for each occurrence from fluoro, chloro, cyano, hydroxyl, carboxyl, 5- to 10-membered heteroaryl, (C₁-C₆)alkyl, (C₁-C₆)haloalkyl, (C₁-C₆)hydroxyalkyl, (C₁-C₆)alkoxyalkyl, (C₁-C₆)carboxyalkyl, (C₁-C₆)alkylsulfonyl, NR¹⁴SO₂R¹⁵, and C(O)NR¹¹R¹²;
R⁶ is hydrogen or (C₁-C₆)alkyl;
R⁷ is hydrogen or (C₁-C₆)alkyl; or R⁷ and Y taken together with the nitrogen atom to which they are attached form a 4- to 7-membered heterocycloalkyl;
R¹¹ and R¹² are independently for each occurrence selected from hydrogen, (C₁-C₆)alkyl, and (C₃-C₈)cycloalkyl; or R¹ and R¹² taken together with the atoms to which they are attached form a 4- to 7-membered heterocycloalkyl;
R¹³ is independently for each occurrence selected from (C₁-C₆)alkyl and (C₃-C₈)cycloalkyl; and
R¹⁴ is independently for each occurrence selected from hydrogen, (C₁-C₆)alkyl, and (C₃-C₈)cycloalkyl, and R¹⁵ is independently for each occurrence selected from (C₁-C₆)alkyl and (C₃-C₈)cycloalkyl; or R¹⁴ and R¹⁵ taken together with the atoms to which they are attached form a 4- to 7-membered heterocycloalkyl.

2. The compound of claim 1, wherein Q is O.

3. The compound of claim 1, wherein Q is S.

4. A compound of Formula (I): 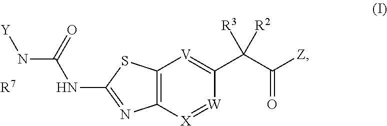
or a pharmaceutically acceptable salt thereof;
wherein:
V, W, and X are independently N or CR¹; provided that at least one of V, W, and X is CR¹;
Y is (C₁-C₆)alkyl, (C₃-C₈)cycloalkyl, 4- to 7-membered heterocycloalkyl, phenyl, or 5- or 6-membered heteroaryl; each of which is optionally substituted with one, two, or three substituents selected independently for each occurrence from fluoro, chloro, cyano, hydroxyl, (C₁-C₆)alkyl, (C₃-C₈)cycloalkyl, (C₁-C₆)haloalkyl, (C₁-C₆)hydroxyalkyl, (C₁-C₆)alkoxyalkyl, and phenyl: each of which substituents is optionally substituted with one, two, or three substituents selected independently for each occurrence from fluoro, (C₁-C₆)haloalkyl, (C₁-C₆)hydroxyalkyl, and (C₁-C₆)alkoxyalkyl;
Z is NR⁴R⁵ or OR⁶;
R¹ is independently for each occurrence H, (C₁-C₆)alkyl, (C₁-C₆)haloalkyl, (C₁-C₆)hydroxyalkyl, (C₁-C₆)alkoxyalkyl, fluoro, chloro, cyano, hydroxyl, or (C₁-C₆)alkoxy;
R² is (C₁-C₆)haloalkyl, (C₁-C₆)alkyl, or (C₃-C₈)cycloalkyl;
R³ is hydroxyl, (C₁-C₆)alkoxyalkyl, or —NH₂; or R² and R³ together with the carbon atom to which they are attached form a (C₃-C₈)cycloalkyl or a 4- to 7-membered heterocycloalkyl; wherein (C₃-C₈)cycloalkyl or 4- to 7-membered heterocycloalkyl is optionally substituted with one, two, or three substituents selected independently for each occurrence from fluoro, chloro, cyano, hydroxyl, (C₁-C₆)alkyl, (C₁-C₆)haloalkyl, (C₁-C₆)hydroxyalkyl, and (C₁-C₆)alkoxyalkyl;
R⁴ is hydrogen, (C₁-C₆)alkyl, (C₃-C₈)cycloalkyl, 4- to 7-membered heterocycloalkyl, phenyl, or 5- or 6-membered heteroaryl; wherein (C₁-C₆)alkyl is optionally substituted with one, two or three substituents selected independently for each occurrence from R^{4a}, halo, hydroxyl, cyano, carboxy, (C₁-C₆)alkylsulfonyl, and (C₁-C₆)alkoxy; and wherein (C₃-C₈)cycloalkyl, 4- to 7-membered heterocycloalkyl, phenyl, or 5- or 6-membered heteroaryl is optionally substituted with one, two, or three substituents selected independently for each occurrence from fluoro, chloro, cyano, hydroxyl, (C₁-C₆)alkyl, (C₁-C₆)haloalkyl, (C₁-C₆)hydroxyalkyl, and (C₁-C₆)alkoxyalkyl;
R^{4a} is (C₃-C₈)cycloalkyl, phenyl, or 4- to 7-membered heterocycloalkyl, each of which is optionally substituted with one, two, or three substituents selected independently for each occurrence from fluoro, chloro, cyano, hydroxyl, (C₁-C₆)alkyl, (C₁-C₆)haloalkyl, (C₁-C₆)hydroxyalkyl, and (C₁-C₆)alkoxyalkyl;
R⁵ is hydrogen or (C₁-C₆)alkyl; or R⁴ and R⁵ taken together with the nitrogen atom to which they are attached form an optionally 4- to 7-membered heterocycloalkyl; wherein 4- to 7-membered heterocycloalkyl is optionally substituted with one, two, or three substituents selected independently for each occurrence from fluoro, chloro, cyano, hydroxyl, carboxyl, (C₁-C₆)alkyl, (C₁-C₆)haloalkyl, (C₁-C₆)hydroxyalkyl, and (C₁-C₆)alkoxyalkyl;
R⁶ is hydrogen or (C₁-C₆)alkyl; and
R⁷ is hydrogen or (C₁-C₆)alkyl; or R⁷ and Y taken together with the nitrogen atom to which they are attached form a 4- to 7-membered heterocycloalkyl.

5. A compound of Formula (I): 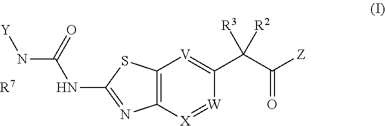
or a pharmaceutically acceptable salt thereof;
wherein:
V, W, and X are independently N or CR¹; provided that at least one of V, W, and X is CR¹;
Y is (C₁-C₆)alkyl, (C₃-C₈)cycloalkyl, 4- to 7-membered heterocycloalkyl, phenyl, or 5- or 6-membered heteroaryl; each of which is optionally substituted with one, two, or three substituents selected independently for each occurrence from fluoro, chloro, cyano, hydroxyl, (C₁-C₆)alkyl, (C₁-C₆)haloalkyl, (C₁-C₆)hydroxyalkyl, (C₁-C₆)alkoxyalkyl, and phenyl;
Z¹ is NR⁴R⁵ or OR⁶;
R¹ is independently for each occurrence H, (C₁-C₆)alkyl, (C₁-C₆)haloalkyl, (C₁-C₆)hydroxyalkyl, (C₁-C₆)alkoxyalkyl, fluoro, chloro, cyano, hydroxyl, or (C₁-C₆)alkoxy;
R² is (C₁-C₆)haloalkyl or (C₁-C₆)alkyl;
R³ is hydroxyl; or R² and R³ together with the carbon atom to which they are attached form a (C₃-C₈)cycloalkyl or a 4- to 7-membered heterocycloalkyl; wherein (C₃-C₈)cycloalkyl or 4- to 7-membered heterocycloalkyl is optionally substituted with one, two, or three substituents selected independently for each occurrence from fluoro, chloro, cyano, hydroxyl, (C₁-C₆)alkyl, (C₁-C₆)haloalkyl, (C₁-C₆)hydroxyalkyl, and (C₁-C₆)alkoxyalkyl;
R⁴ is hydrogen, (C₁-C₆)alkyl, (C₃-C₈)cycloalkyl, 4- to 7-membered heterocycloalkyl, phenyl, or 5- or 6-membered heteroaryl; wherein (C₁-C₆)alkyl is optionally substituted with one, two or three substituents selected independently for each occurrence from R^{4a}, halo, hydroxyl, and (C₁-C₆)alkoxy; and wherein (C₃-C₈)cycloalkyl, 4- to 7-membered heterocycloalkyl, phenyl, or 5- or 6-membered heteroaryl is optionally substituted with one, two, or three substituents selected independently for each occurrence from fluoro, chloro, cyano, hydroxyl, (C₁-C₆)alkyl, (C₁-C₆)haloalkyl, (C₁-C₆)hydroxyalkyl, and (C₁-C₆)alkoxyalkyl;
R^{4a} is (C₃-C₈)cycloalkyl, phenyl, or 4- to 7-membered heterocycloalkyl; each of which is optionally substituted with one, two, or three substituents selected independently for each occurrence from fluoro, chloro, cyano, hydroxyl, (C₁-C₆)alkyl, (C₁-C₆)haloalkyl, (C₁-C₆)hydroxyalkyl, and (C₁-C₆)alkoxyalkyl;
R⁵ is hydrogen or (C₁-C₆)alkyl; or R⁴ and R⁵ taken together with the nitrogen atom to which they are attached form an optionally 4- to 7-membered heterocycloalkyl; wherein 4- to 7-membered heterocycloalkyl is optionally substituted with one, two, or three substituents selected independently for each occurrence from fluoro, chloro, cyano, hydroxyl, (C₁-C₆)alkyl, (C₁-C₆)haloalkyl, (C₁-C₆)hydroxyalkyl, and (C₁-C₆)alkoxyalkyl;
R⁶ is hydrogen or (C₁-C₆)alkyl; and
R⁷ is hydrogen or (C₁-C₆)alkyl; or R⁷ and Y taken together with the nitrogen atom to which they are attached form a 4- to 7-membered heterocycloalkyl.

6. The compound of claim 1, 2, 4, or 5, having the structure of formula Ia: 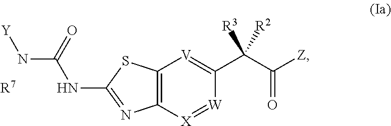
or a pharmaceutically acceptable salt thereof.

7. The compound of claim 1, 2, 4, or 5, having the structure of formula Ib: 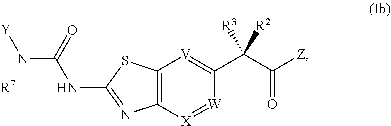
or a pharmaceutically acceptable salt thereof.

8. A compound of Formula (IIa): 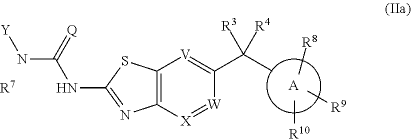
or a pharmaceutically acceptable salt thereof;
wherein:
A is 5- to 10-membered heteroaryl;
Q is S or O;
V, W, and X are independently N or CR¹; provided that at least one of V, W, and X is CR¹;
Y is (C₁-C₆)alkyl, (C₃-C₈)cycloalkyl, 4- to 7-membered heterocycloalkyl, phenyl, or 5- or 6-membered heteroaryl; each of which is optionally substituted with one, two, or three substituents selected independently for each occurrence from fluoro, chloro, cyano, hydroxyl, (C₁-C₆)alkyl, (C₃-C₈)cycloalkyl, (C₁-C₆)haloalkyl, (C₁-C₆)hydroxyalkyl, (C₁-C₆)alkoxyalkyl, and phenyl; each of which substituents is optionally substituted with one, two, or three substituents selected independently for each occurrence from fluoro, (C₁-C₆)alkyl, (C₁-C₆)haloalkyl, (C₁-C₆)hydroxyalkyl, and (C₁-C₆)alkoxyalkyl;
R¹ is independently for each occurrence H, (C₁-C₆)alkyl, (C₁-C₆)haloalkyl, (C₁-C₆)hydroxyalkyl, (C₁-C₆)alkoxyalkyl, fluoro, chloro, cyano, hydroxyl, or (C₁-C₆)alkoxy;
R² is (C₁-C₆)haloalkyl, (C₁-C₆)alkyl, or (C₁-C₆)cycloalkyl;
R³ is hydroxyl, (C₁-C₆)alkyl, (C₁-C₆)alkoxyalkyl, (C₁-C₆)hydroxyalkyl, NR¹¹R¹², NC(O)R¹³, OC(O)NR¹¹R¹², NR¹¹SO₂R¹²; or R² and R³ together with the carbon atom to which they are attached form a (C₃-C₆)cycloalkyl or a 4- to 7-membered heterocycloalkyl;
wherein (C₃-C₈)cycloalkyl or 4- to 7-membered heterocycloalkyl is optionally substituted with one, two, or three substituents selected independently for each occurrence from fluoro, chloro, cyano, hydroxyl, (C₁-C₆)alkyl, (C₁-C₆)haloalkyl, (C₁-C₆)hydroxyalkyl, and (C₁-C₆)alkoxyalkyl;
R⁷ is hydrogen or (C₁-C₆)alkyl; or R⁷ and Y taken together with the nitrogen atom to which they are attached form a 4- to 7-membered heterocycloalkyl;
R⁸, R⁹ and R¹⁰ are independently for each occurrence H, (C₁-C₆)alkyl, or carboxy; wherein each (C₁-C₆)alkyl is optionally substituted with one, two, or three substituents selected independently for each occurrence from the group consisting of (C₁-C₆)alkoxy, hydroxy, fluoro, chloro, cyano, and NR¹¹R¹²,
R¹¹ and R¹² are independently for each occurrence selected from hydrogen, (C₁-C₆)alkyl, and (C₃-C₈)cycloalkyl; or R¹¹ and R¹² taken together with the atoms to which they are attached form a 4- to 7-membered heterocycloalkyl;
R¹³ is independently for each occurrence selected from (C₁-C₆)alkyl and (C₃-C₈)cycloalkyl; and
R¹⁴ is independently for each occurrence selected from hydrogen, (C₁-C₆)alkyl, and (C₃-C₈)cycloalkyl, and R¹⁵ is independently for each occurrence selected from (C₁-C₆)alkyl and (C₃-C₈)cycloalkyl; or R¹⁴ and R¹⁵ taken together with the atoms to which they are attached form a 4- to 7-membered heterocycloalkyl.

9. The compound of claim 8, wherein A is a 5- or 6-membered heteroaryl.

10. The compound of claim 8 or 9, wherein A comprises 1, 2, 3, or 4 nitrogen atoms.

11. The compound of any one of claims 8 to 10, wherein A is diazolyl, triazolyl, imidazolyl, tetrazolyl, oxadiazolyl, thiadiazole, diazole, pyridazinyl, or pyrzinlyl.

12. The compound of any one of claims 8 to 11, wherein R⁸, R⁹ and R¹⁰ are independently for each occurrence H, (C₁-C₆)alkyl, or carboxy; and each (C₁-C₆)alkyl is optionally substituted with one, two, or three instances of fluoro.

13. The compound of any one of claims 8 to 12, wherein Q is O.

14. The compound of any one of claims 8 to 12, wherein Q is S.

15. A compound of Formula (IIb): 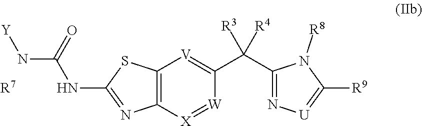
or a pharmaceutically acceptable salt thereof;
wherein:
V, W, and X are independently N or CR¹; provided that at least one of V, W, and X is CR¹;
Y is (C₁-C₆)alkyl, (C₃-C₈)cycloalkyl, 4- to 7-membered heterocycloalkyl, phenyl, or 5- or 6-membered heteroaryl; each of which is optionally substituted with one, two, or three substituents selected independently for each occurrence from fluoro, chloro, cyano, hydroxyl, (C₁-C₆)alkyl, (C₃-C₈)cycloalkyl, (C₁-C₆)haloalkyl, (C₁-C₆)hydroxyalkyl, (C₁-C₆)alkoxyalkyl, and phenyl; each of which substituents is optionally substituted with one, two, or three substituents selected independently for each occurrence from fluoro, (C₁-C₆)haloalkyl, (C₁-C₆)hydroxyalkyl, and (C₁-C₆)alkoxyalkyl;
U is N or CR¹⁰;
R¹ is independently for each occurrence H, (C₁-C₆)alkyl, (C₁-C₆)haloalkyl, (C₁-C₆)hydroxyalkyl, (C₁-C₆)alkoxyalkyl, fluoro, chloro, cyano, hydroxyl, or (C₁-C₆)alkoxy;
R² is (C₁-C₆)haloalkyl, (C₁-C₆)alkyl, or (C₃-C₈)cycloalkyl;
R³ is hydroxyl, (C₁-C₆)alkoxyalkyl, or —NH₂; or R² and R³ together with the carbon atom to which they are attached form a (C₁-C₆)cycloalkyl or a 4- to 7-membered heterocycloalkyl; wherein (C₃-C₈)cycloalkyl or 4- to 7-membered heterocycloalkyl is optionally substituted with one, two, or three substituents selected independently for each occurrence from fluoro, chloro, cyano, hydroxyl, (C₁-C₆)alkyl, (C₁-C₆)haloalkyl, (C₁-C₆)hydroxyalkyl, and (C₁-C₆)alkoxyalkyl;
R⁷ is hydrogen or (C₁-C₆)alkyl; or R⁷ and Y taken together with the nitrogen atom to which they are attached form a 4- to 7-membered heterocycloalkyl;
R⁸, R⁹ and R¹⁰ are independently for each occurrence H or (C₁-C₆)alkyl; wherein each (C₁-C₆)alkyl is optionally substituted with one, two, or three substituents selected independently for each occurrence from the group consisting of hydroxy, (C₁-C₆)hydroxyalkyl, (C₁-C₆)alkoxyalkyl, (C₁-C₆)alkoxy, fluoro, chloro, cyano, and NR¹¹R¹²; and
R¹¹ and R¹² are each independently hydrogen or (C₁-C₆)alkyl; or R¹¹ and R¹² taken together with the nitrogen atom to which they are attached form a 4- to 7-membered heterocycloalkyl.

16. The compound of claim 15, wherein R⁸ is H.

17. The compound of claim 15 or 16, wherein R⁹ is (C₁-C₆)alkyl.

18. The compound of claim 17, wherein R⁹ is methyl.

19. The compound of any one of claims 15 to 18, wherein U is N.

20. The compound of any one of claims 15 to 18, wherein U is CR¹⁰.

21. The compound of claim 20, wherein R¹⁰ is H.

22. The compound of any one of claims 1-21, wherein V, W, and X are each CR¹.

23. The compound of any one of claims 1-21, wherein V is N; and W and X are each CR¹.

24. The compound of any one of claims 1-21, wherein W is N, and V and X are each CR¹.

25. The compound of any one of claims 1-21, wherein X is N; and V and W are each CR¹.

26. The compound of any one of claims 1-21, wherein V and W are each N; and X is CR¹.

27. The compound of any one of claims 1-26, wherein R′ is independently for each occurrence selected from hydrogen, methyl, trifluoromethyl, fluoro, and methoxy.

28. The compound of any one of claims 1-27, wherein one and only one instance of R¹ is selected from methyl, trifluoromethyl, fluoro, and methoxy; and the remaining instances of R¹ are hydrogen.

29. The compound of any one of claims 1-27, wherein each occurrence of R¹ is hydrogen.

30. The compound of any one of claims 1 to 29, wherein Y is optionally substituted with fluoro or chloro.

31. The compound of any one of claims 1 to 30, wherein Y is optionally substituted phenyl.

32. The compound of claim 23, wherein Y is phenyl optionally substituted with 1 or 2 substituents independently selected from fluoro and cyano.

33. The compound of claim 24, wherein Y is 2,3-difluorophenyl, 2,4-difluorophenyl, 4-fluorophenyl, or 4-cyanophenyl.

34. The compound of claim 23, wherein Y is 4-fluoro phenyl.

35. The compound of any one of claims 1-30, wherein Y is optionally substituted 2-pyridyl.

36. The compound of claim 35, wherein Y is 4-fluoro-2-pyridyl.

37. The compound of any one of claims 1-29, wherein Y is 5-thiazolyl or 5-isothiazolyl, each of which is optionally substituted with methyl.

38. The compound of any one of claims 1-29, wherein Y is optionally substituted (C₃-C₈)cycloalkyl.

39. The compound of claim 38, wherein Y is bicyclo[1.1.1]pent-1-yl or cyclopentyl, each of which is optionally substituted with one or two fluoro substituents.

40. The compound of any one of claims 1-29, wherein Y is methyl substituted with cyclopropyl, cyclobutyl, or cyclopentyl, each of which is optionally substituted with one or two substituents independently selected from fluoro, trifluoromethyl, and methyl.

41. The compound of any one of claims 1 to 40, wherein R² is (C₁-C₆)haloalkyl.

42. The compound of claim 41, wherein R² is (C₁-C₆)fluoroalkyl.

43. The compound of claim 42, wherein R² is trifluoromethyl.

44. The compound of claim 42, wherein R² is difluoromethyl.

45. The compound of claim 44, wherein R² is 2,2,2-trifluoroethyl.

46. The compound of any one of claims 1 to 40, wherein R² is cyclopropyl.

47. The compound of any one of claims 1 to 40, wherein R² is (C₁-C₆)alkyl.

48. The compound of claim 47, wherein R² is methyl.

49. The compound of claim 48, wherein R² is ethyl.

50. The compound of any one of claims 1 to 49, wherein R³ is hydroxyl.

51. The compound of any one of claims 1 to 49, wherein R³ is methoxy.

52. The compound of any one of claims 1 to 49, wherein R³ is —NH₂.

53. The compound of any one of claims 1 to 49, wherein R³ is NR¹¹R¹², NC(O)R¹³, OC(O)NR¹¹R¹², or NR¹¹SO₂R¹².

54. The compound of any one of claims 1 to 40, wherein R² and R³ together with the carbon atom to which they are attached form an optionally substituted (C₃-C₈)cycloalkyl.

55. The compound of claim 54, wherein R² and R³ together with the carbon atom to which they are attached form a cyclopentyl.

56. The compound of claim 54, wherein R² and R³ together with the carbon atom to which they are attached form a cyclobutyl.

57. The compound of claim 54, wherein R² and R³ together with the carbon atom to which they are attached form a cyclopropyl.

58. The compound of any one of claims 1 to 40, wherein R² and R³ together with the carbon atom to which they are attached form an optionally substituted 4- to 7-membered heterocycloalkyl.

59. The compound of claim 58, wherein R² and R³, together with the carbon atom to which they are attached, form an optionally substituted 5-membered heterocycloalkyl.

60. The compound of claim 59, wherein R² and R³ together with the carbon atom to which they are attached form a pyrrolidinyl or a tetrahydrofuranyl.

61. The compound of any one of claims 1 to 3 and 12 to 49, wherein Z is NR⁴R³.

62. The compound of claim 61, wherein R⁴ is hydrogen.

63. The compound of claim 61, wherein R⁴ is (C₁-C₆)alkyl optionally substituted with one, two, or three substituents independently selected from halo and R^{4a}.

64. The compound of claim 61, wherein R⁴ is (C₁-C₆)alkyl optionally substituted with one, two, or three substituents independently selected from fluoro, (C₁-C₆)alkylsulfonyl, cyano, carboxy, NR¹¹R¹², or C(O)NR¹¹R¹².

65. The compound of claim 64, wherein R⁴ is (C₁-C₃)alkyl optionally substituted with fluoro, (C₁-C₆)alkylsulfonyl, cyano, or carboxy.

66. The compound of claim 65, wherein R⁴ is (C₁-C₆)alkyl substituted with one, two, or three instances of fluoro.

67. The compound of any one of claims 64 to 66, wherein R⁴ is methyl, ethyl or isopropyl, each of which is optionally substituted with fluoro, carboxy, methylsulfonyl, or cyano.

68. The compound of claim 63, wherein R⁴ is ethyl or isopropyl.

69. The compound of claim 63, wherein R⁴ is (C₁-C₆)alkyl substituted with one, two, or three substituents independently selected from R^{4a}.

70. The compound of claim 69 or 70, wherein R⁴ is methyl substituted with one, two, or three substituents independently selected from R^{4a}.

71. The compound of claim 69 or 70, wherein R^{4a} is (C₁-C₆)cycloalkyl optionally substituted with one, two, or three substituents independently selected from halo, cyano, hydroxyl, (C₁-C₆)alkyl, (C₁-C₆)haloalkyl, and (C₁-C₆)alkoxy.

72. The compound of claim 69 or 70, wherein R^{4a} is (C₃-C₈)cycloalkyl optionally substituted with one, two, or three substituents independently selected from halo, cyano, carboxy, hydroxyl, (C₁-C₆)alkyl, (C₁-C₆)haloalkyl, and (C₁-C₆)alkoxy.

73. The compound of claim 72, wherein R^{4a} is (C₃-C₈)cycloalkyl optionally substituted with fluoro.

74. The compound of any one of claims 69 to 73, wherein the optionally substituted (C₃-C₈)cycloalkyl is cyclopropyl.

75. The compound of claim 72, wherein R^{4a} is cyclopentyl optionally substituted with hydroxy.

76. The compound of claim 72, wherein R^{4a} is cyclopropyl substituted with carboxy.

77. The compound of claim 72, wherein R^{4a} is cyclopropyl.

78. The compound of claim 69 to 70, wherein R^{4a} is phenyl optionally substituted with one, two, or three substituents independently selected from halo, hydroxyl, and (C₁-C₆)alkoxy.

79. The compound of claim 78, wherein R^{4a} is phenyl optionally substituted with one, two, or three substituents independently selected from (C₁-C₆)alkoxy.

80. The compound of claim 79, wherein R^{4a} is phenyl optionally substituted with methoxy.

81. The compound of claim 69 or 70, wherein R^{4a} is 4- to 7-membered heterocycloalkyl optionally substituted with one, two, or three substituents independently selected from halo, cyano, hydroxyl, (C₁-C₆)alkyl, (C₁-C₆)haloalkyl, and (C₁-C₆)alkoxy.

82. The compound of claim 81, wherein R^{4a} is tetrahydrofuranyl, oxetanyl, piperidinyl, or pyrrolidinyl, each of which is optionally substituted with one, two, or three substituents independently selected from halo, cyano, hydroxyl, (C₁-C₆)alkyl, (C₁-C₆)haloalkyl, and (C₁-C₆)alkoxy.

83. The compound of claim 61, wherein R⁴ is (C₃-C₈)cycloalkyl optionally substituted with one, two, or three substituents independently selected from halo, hydroxyl, carboxy, (C₁-C₆)alkyl, and (C₁-C₆)alkoxy.

84. The compound of claim 61, wherein R⁴ is (C₃-C₈)cycloalkyl optionally substituted with one, two, or three substituents independently selected from halo, hydroxyl, and (C₁-C₆)alkoxy.

85. The compound of claim 84, wherein (C₃-C₈)cycloalkyl is optionally substituted with one, two or three instances of hydroxyl.

86. The compound of claim 84, wherein (C₃-C₈)cycloalkyl is optionally substituted with one, two, or three instances of fluoro.

87. The compound of claim 84, wherein (C₃-C₈)cycloalkyl is optionally substituted with (C₁-C₆)alkylsulfonyl, (C₁-C₆)alkylcarbonyl C(O)NR¹¹R¹², NR¹¹R¹², NC(O)OR¹³, NC(O)R¹³, OC(O)NR¹¹R¹², or SO₂NR¹¹R¹².

88. The compound of claim any one of claims 84 to 87, wherein (C₃-C₈)cycloalkyl is cyclopropyl, cyclobutyl, or cyclopentyl.

89. The compound of claim 88, wherein (C₃-C₈)cycloalkyl is cyclobutyl.

90. The compound of claim 61, wherein R⁴ is 4- to 7-membered heterocycloalkyl optionally substituted with one, two, or three substituents independently selected from halo, hydroxyl, (C₁-C₆)alkoxy, carboxy, and (C₁-C₆)alkyl.

91. The compound of claim 90, wherein R⁴ is oxetanyl, piperidinyl, pyrrolidinyl, or tetrahydrofuranyl, each of which is optionally substituted with one, two, or three substituents independently selected from hydroxyl and carboxyl.

92. The compound of claim 91, wherein R⁴ is oxetane or tetrahydrofuran.

93. The compound of claim 61, wherein R⁴ is phenyl.

94. The compound of claim 61, wherein R⁴ is 5- or 6-membered heteroaryl.

95. The compound of claim 94, wherein R⁴ is pyridyl.

96. The compound of claim 94, wherein R⁴ is diazolyl.

97. The compound of any one of claims 61 to 96, wherein R⁵ is hydrogen.

98. The compound of any one of claims 61 to 96, wherein R⁵ is (C₁-C₆)alkyl.

99. The compound of claim 98, wherein R⁵ is ethyl.

100. The compound of claim 61, wherein R⁴ and R⁵ taken together with the nitrogen atom to which they are attached form a 4- to 7-membered heterocycloalkyl optionally substituted with one, two, or three substituents independently selected from fluoro, hydroxy, carboxy, cyano, (C₁-C₆)alkyl, (C₁-C₆)alkoxycarbonyl, (C₁-C₆)alkylcarbonyl, (C₁-C₆)alkylsulfonyl, (C₁-C₆)fluoroalkyl, and (C₃-C₈)cycloalkyl.

101. The compound of claim 61, wherein R⁴ and R⁵ taken together with the nitrogen atom to which they are attached form a 4- to 7-membered heterocycloalkyl optionally substituted with 5- to 10-membered heteroaryl, (C₁-C₆)carboxyalkyl, (C₁-C₆)alkylsulfonyl, NR¹⁴SO₂R¹⁵, or C(O)NR¹¹R¹².

102. The compound of claim 100 or 101, wherein R⁴ and R⁵ taken together with the nitrogen atom to which they are attached form azetidiyl, pyrrolidiyl, piperidinyl, piperazinyl, tetrahydropyranyl, morpholinyl, 3-azabicyclo[3.1.0]hexanyl, 2-oxa-6-aza-6-spiro[3.3]heptyl, 8-oxa-3-azabicyclo[3.2.1]oct-3-yl, and 1,7-diaza-1-indanyl.

103. The compound of claim 102, wherein R⁴ and R⁵ taken together with the nitrogen atom to which they are attached form azetidiyl.

104. The compound of claim 102, wherein the heterocycloalkyl is morpholino.

105. The compound of any one of claims 1 to 7 and 22 to 104, wherein Z is OR⁶.

106. The compound of claim 105, wherein R⁶ is hydrogen

107. The compound of claim 105, wherein R⁶ is (C₁-C₆)alkyl.

108. The compound of claim 107, wherein R⁶ is methyl or ethyl.

109. The compound of any one of claims 1 to 108, wherein R⁷ is hydrogen.

110. The compound of any one of claims 1 to 108, wherein R⁷ is (C₁-C₆)alkyl.

111. The compound of any one of claims 1 to 29 and 41 to 108, wherein Y and R⁷ taken together with the nitrogen atom to which they are attached form a 4- to 7-membered heterocycloalkyl.

112. The compound of claim 111, wherein the 4- to 7-membered heterocycloalkyl is piperidine.

113. The compound of any one of claims 1 to 112, wherein R¹¹ is hydrogen.

114. The compound of any one of claims 1 to 113, wherein R¹² is hydrogen.

115. The compound of any one of claims 1 to 113, wherein R¹² is (C₁-C₆)alkyl.

116. A compound having the structure: 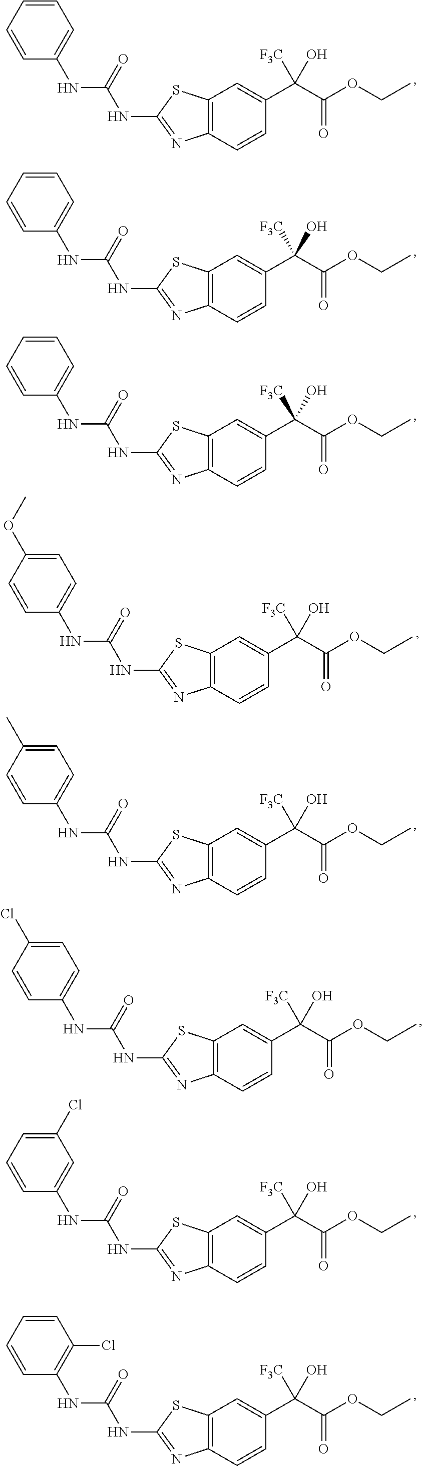 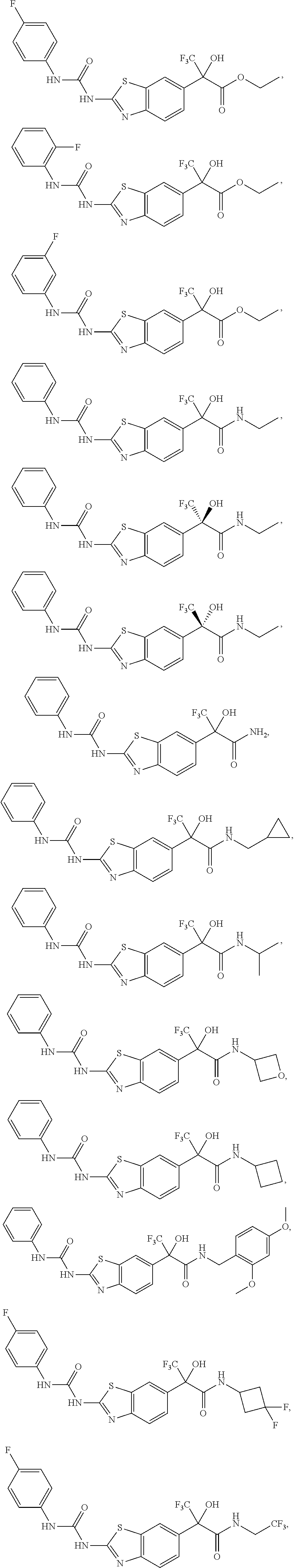 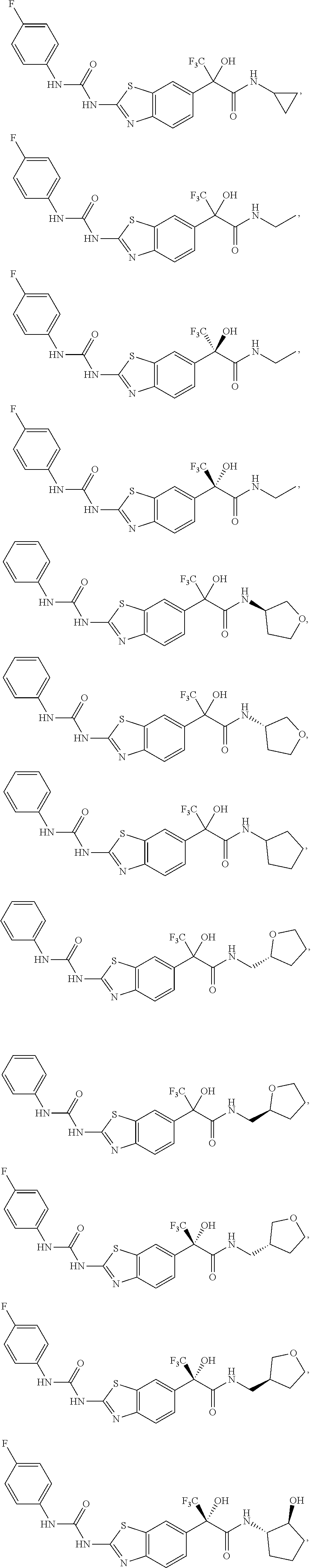 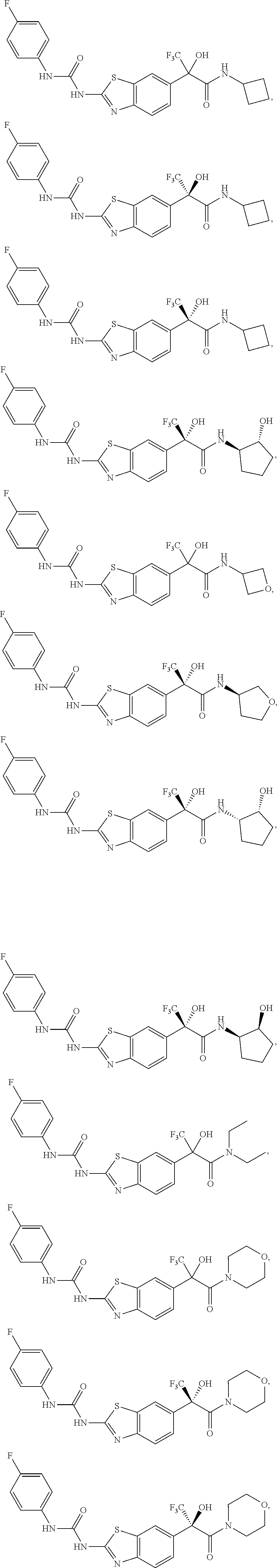 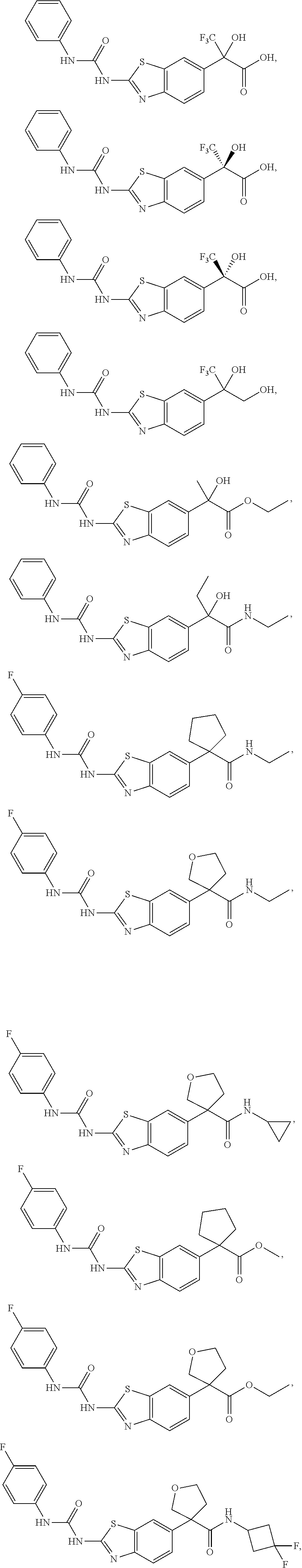 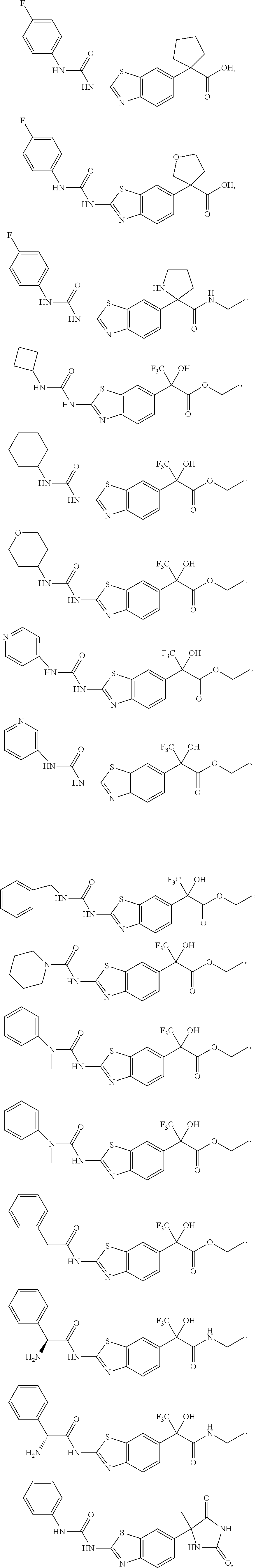 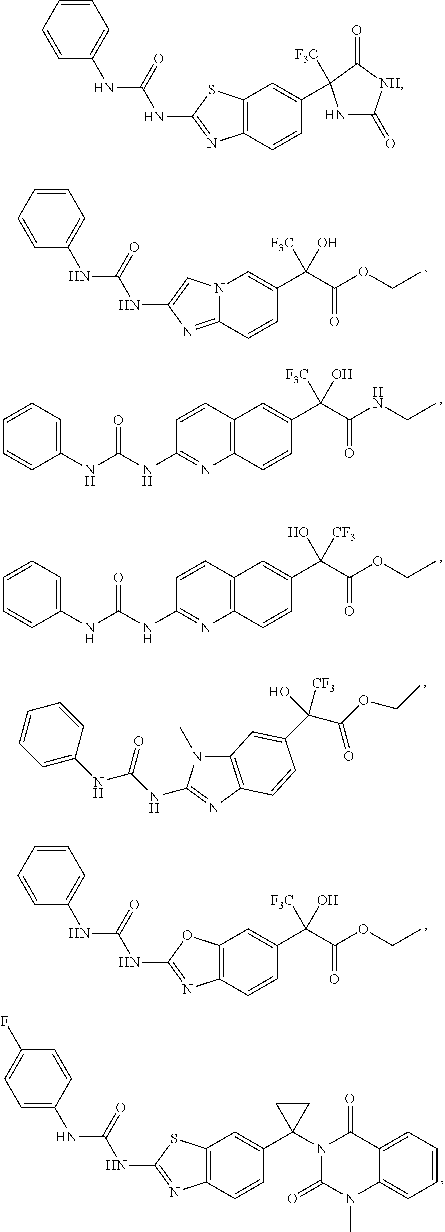 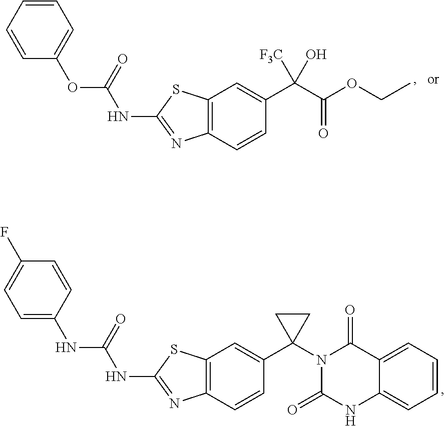 [SMILES: CCOC(=O)C(O)(c1ccc2nc(NC(=O)Oc3ccccc3)sc2c1)C(F)(F)F ; O=C(Nc1ccc(F)cc1)Nc1nc2ccc(C3(n4c(=O)[nH]c5ccccc5c4=O)CC3)cc2s1]
or a pharmaceutically acceptable salt thereof.

117. A compound having the structure: 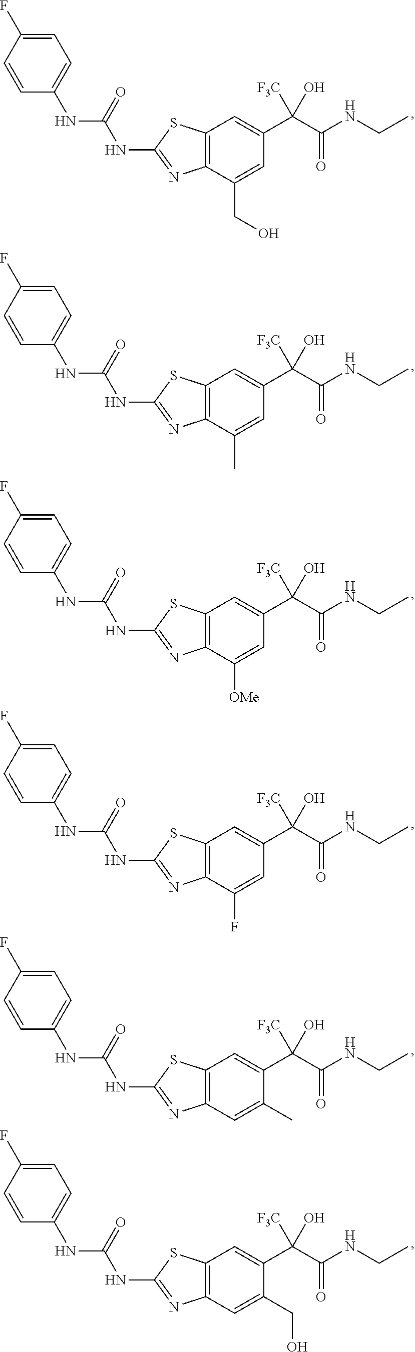 [SMILES: CCNC(=O)C(O)(c1cc(CO)c2nc(NC(=O)Nc3ccc(F)cc3)sc2c1)C(F)(F)F ; CCNC(=O)C(O)(c1cc(C)c2nc(NC(=O)Nc3ccc(F)cc3)sc2c1)C(F)(F)F ; CCNC(=O)C(O)(c1cc(OC)c2nc(NC(=O)Nc3ccc(F)cc3)sc2c1)C(F)(F)F ; CCNC(=O)C(O)(c1cc(F)c2nc(NC(=O)Nc3ccc(F)cc3)sc2c1)C(F)(F)F ; CCNC(=O)C(O)(c1cc2sc(NC(=O)Nc3ccc(F)cc3)nc2cc1C)C(F)(F)F ; CCNC(=O)C(O)(c1cc2sc(NC(=O)Nc3ccc(F)cc3)nc2cc1CO)C(F)(F)F] 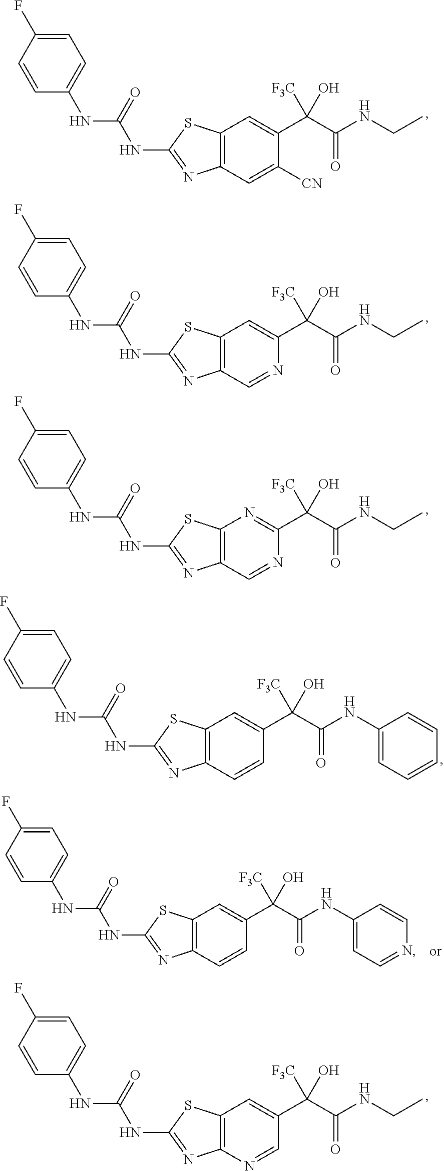 [SMILES: CCNC(=O)C(O)(c1cc2sc(NC(=O)Nc3ccc(F)cc3)nc2cc1C#N)C(F)(F)F ; CCNC(=O)C(O)(c1cc2sc(NC(=O)Nc3ccc(F)cc3)nc2cn1)C(F)(F)F ; CCNC(=O)C(O)(c1ncc2nc(NC(=O)Nc3ccc(F)cc3)sc2n1)C(F)(F)F ; O=C(Nc1ccc(F)cc1)Nc1nc2ccc(C(O)(C(=O)Nc3ccccc3)C(F)(F)F)cc2s1 ; O=C(Nc1ccc(F)cc1)Nc1nc2ccc(C(O)(C(=O)Nc3ccncc3)C(F)(F)F)cc2s1 ; CCNC(=O)C(O)(c1cnc2nc(NC(=O)Nc3ccc(F)cc3)sc2c1)C(F)(F)F]
or a pharmaceutically acceptable salt thereof.

118. A compound having a structure selected from the following table:

| # | Structure |
|---|---|
| 87 | 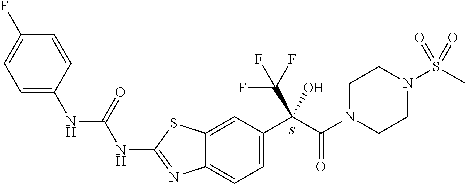 |
| 88 | 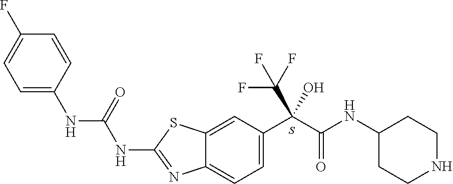 |
| 89 | 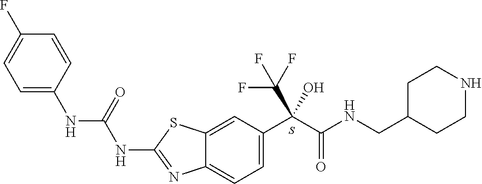 |
| 90 | 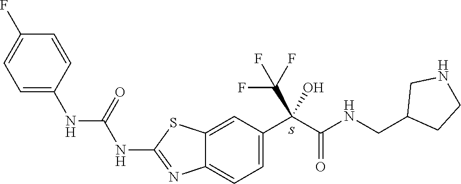 |
| 91 | 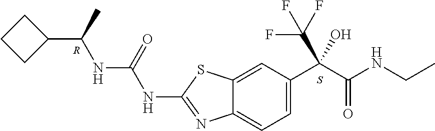 |
| 92 | 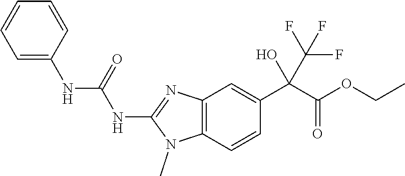 [SMILES: CCOC(=O)C(O)(c1ccc2c(c1)nc(NC(=O)Nc1ccccc1)n2C)C(F)(F)F] |
| 93 | 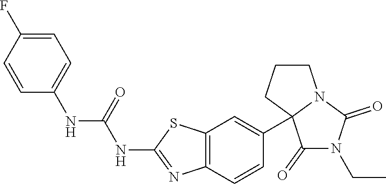 [SMILES: CCN1C(=O)N2CCCC2(c2ccc3nc(NC(=O)Nc4ccc(F)cc4)sc3c2)C1=O] |
| 94 | 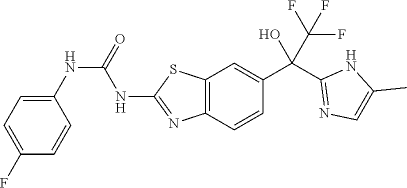 [SMILES: Cc1cnc(C(O)(c2ccc3nc(NC(=O)Nc4ccc(F)cc4)sc3c2)C(F)(F)F)[nH]1] |
| 95 | 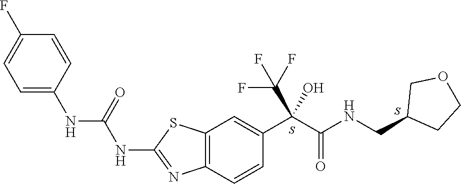 |
| 96 | 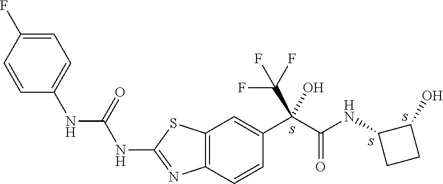 |
| 97 | 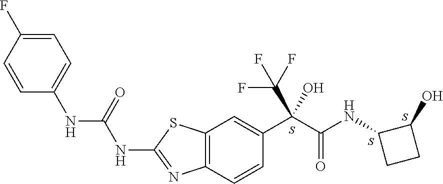 |
| 98 | 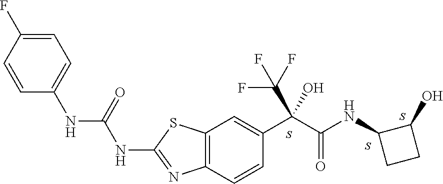 |
| 99 | 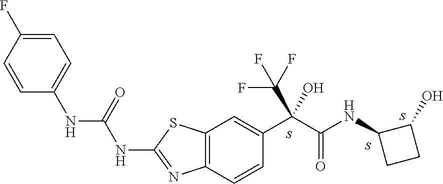 |
| 100 | 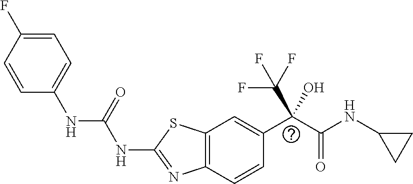 [SMILES: O=C(Nc1ccc(F)cc1)Nc1nc2ccc([C@](O)(C(=O)NC3CC3)C(F)(F)F)cc2s1] |
| 101 | 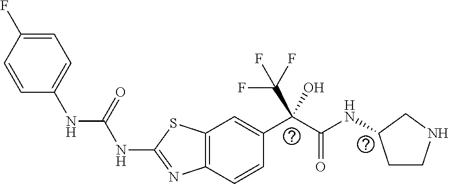 [SMILES: O=C(Nc1ccc(F)cc1)Nc1nc2ccc([C@](O)(C(=O)N[C@H]3CCNC3)C(F)(F)F)cc2s1] |
| 102 | 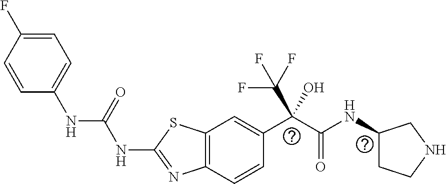 [SMILES: O=C(Nc1ccc(F)cc1)Nc1nc2ccc([C@](O)(C(=O)N[C@@H]3CCNC3)C(F)(F)F)cc2s1] |
| 103 | 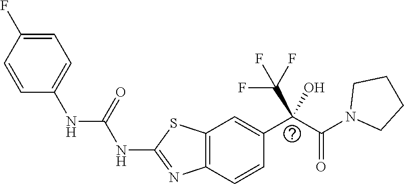 [SMILES: O=C(Nc1ccc(F)cc1)Nc1nc2ccc([C@](O)(C(=O)N3CCCC3)C(F)(F)F)cc2s1] |
| 104 | 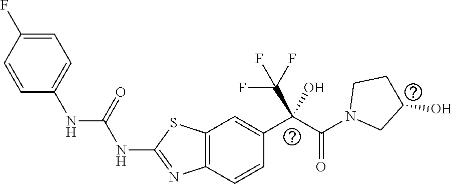 [SMILES: O=C(Nc1ccc(F)cc1)Nc1nc2ccc([C@](O)(C(=O)N3CC[C@H](O)C3)C(F)(F)F)cc2s1] |
| 105 | 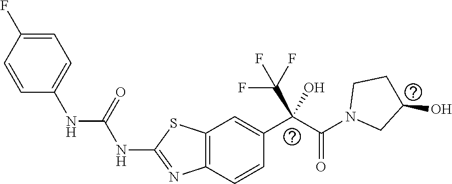 [SMILES: O=C(Nc1ccc(F)cc1)Nc1nc2ccc([C@](O)(C(=O)N3CC[C@@H](O)C3)C(F)(F)F)cc2s1] |
| 106 | 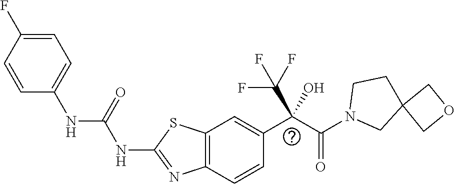 [SMILES: O=C(Nc1ccc(F)cc1)Nc1nc2ccc([C@](O)(C(=O)N3CCC4(COC4)C3)C(F)(F)F)cc2s1] |
| 107 | 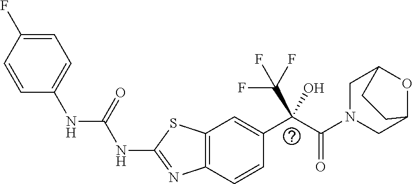 [SMILES: O=C(Nc1ccc(F)cc1)Nc1nc2ccc([C@](O)(C(=O)N3CC4CCC(C3)O4)C(F)(F)F)cc2s1] |
| 108 | 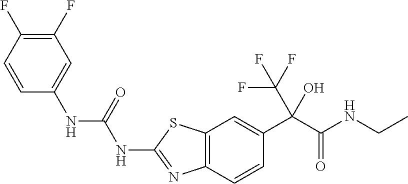 [SMILES: CCNC(=O)C(O)(c1ccc2nc(NC(=O)Nc3ccc(F)c(F)c3)sc2c1)C(F)(F)F] |
| 109 | 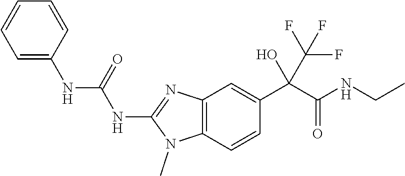 [SMILES: CCNC(=O)C(O)(c1ccc2c(c1)nc(NC(=O)Nc1ccccc1)n2C)C(F)(F)F] |
| 110 | 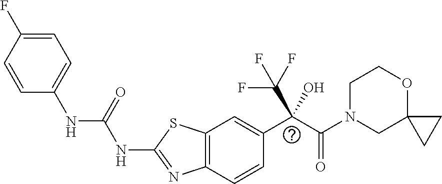 [SMILES: O=C(Nc1ccc(F)cc1)Nc1nc2ccc([C@](O)(C(=O)N3CCOC4(CC4)C3)C(F)(F)F)cc2s1] |
| 111 | 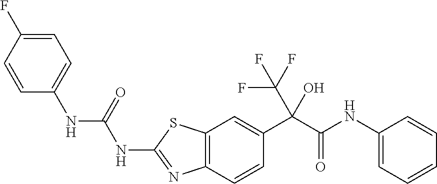 [SMILES: O=C(Nc1ccc(F)cc1)Nc1nc2ccc(C(O)(C(=O)Nc3ccccc3)C(F)(F)F)cc2s1] |
| 112 | 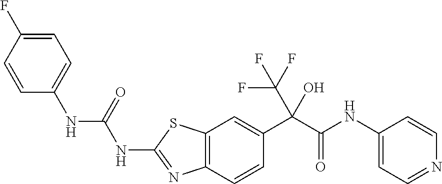 [SMILES: O=C(Nc1ccc(F)cc1)Nc1nc2ccc(C(O)(C(=O)Nc3ccncc3)C(F)(F)F)cc2s1] |
| 113 | 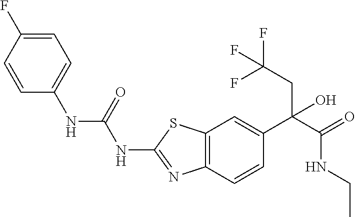 [SMILES: CCNC(=O)C(O)(CC(F)(F)F)c1ccc2nc(NC(=O)Nc3ccc(F)cc3)sc2c1] |
| 114 | 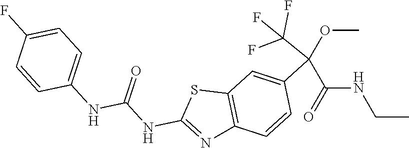 [SMILES: CCNC(=O)C(OC)(c1ccc2nc(NC(=O)Nc3ccc(F)cc3)sc2c1)C(F)(F)F] |
| 115 | 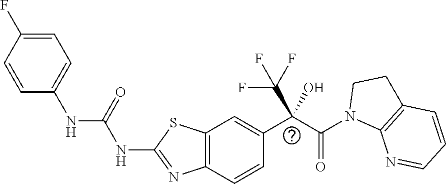 [SMILES: O=C(Nc1ccc(F)cc1)Nc1nc2ccc([C@](O)(C(=O)N3CCc4cccnc43)C(F)(F)F)cc2s1] |
| 116 | 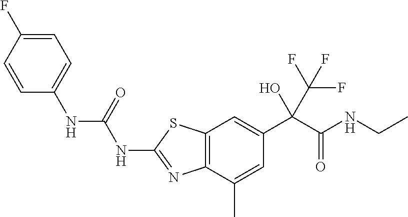 [SMILES: CCNC(=O)C(O)(c1cc(C)c2nc(NC(=O)Nc3ccc(F)cc3)sc2c1)C(F)(F)F] |
| 117 | 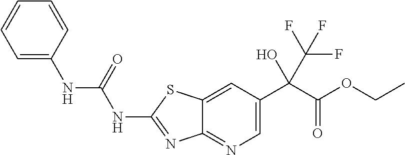 [SMILES: CCOC(=O)C(O)(c1cnc2nc(NC(=O)Nc3ccccc3)sc2c1)C(F)(F)F] |
| 118 | 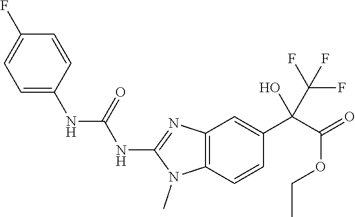 [SMILES: CCOC(=O)C(O)(c1ccc2c(c1)nc(NC(=O)Nc1ccc(F)cc1)n2C)C(F)(F)F] |
| 119 | 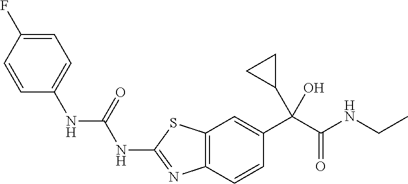 [SMILES: CCNC(=O)C(O)(c1ccc2nc(NC(=O)Nc3ccc(F)cc3)sc2c1)C1CC1] |
| 120 | 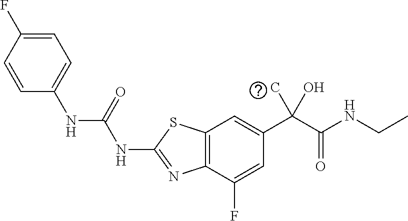 [SMILES: CCNC(=O)C(C)(O)c1cc(F)c2nc(NC(=O)Nc3ccc(F)cc3)sc2c1] |
| 121 | 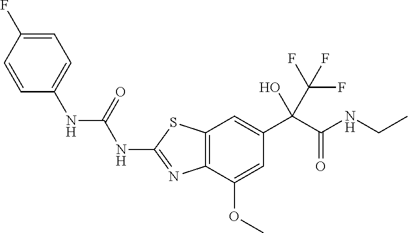 [SMILES: CCNC(=O)C(O)(c1cc(OC)c2nc(NC(=O)Nc3ccc(F)cc3)sc2c1)C(F)(F)F] |
| 122 | 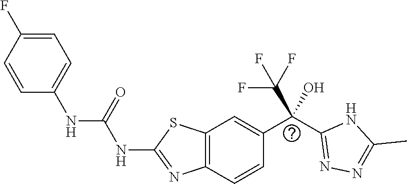 [SMILES: Cc1nnc([C@@](O)(c2ccc3nc(NC(=O)Nc4ccc(F)cc4)sc3c2)C(F)(F)F)[nH]1] |
| 123 | 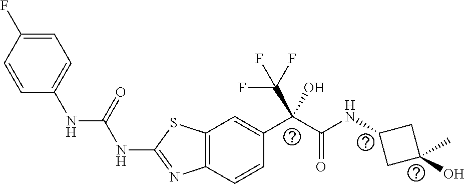 [SMILES: C[C@]1(O)C[C@H](NC(=O)[C@@](O)(c2ccc3nc(NC(=O)Nc4ccc(F)cc4)sc3c2)C(F)(F)F)C1] |
| 124 | 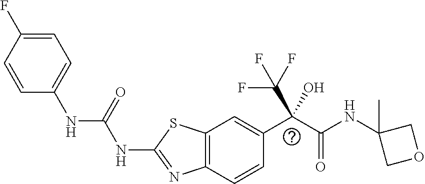 [SMILES: CC1(NC(=O)[C@@](O)(c2ccc3nc(NC(=O)Nc4ccc(F)cc4)sc3c2)C(F)(F)F)COC1] |
| 125 | 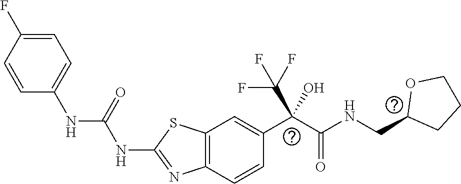 [SMILES: O=C(Nc1ccc(F)cc1)Nc1nc2ccc([C@](O)(C(=O)NC[C@@H]3CCCO3)C(F)(F)F)cc2s1] |
| 126 | 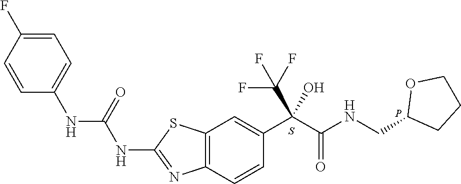 |
| 127 | 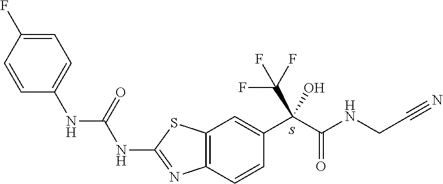 |
| 128 | 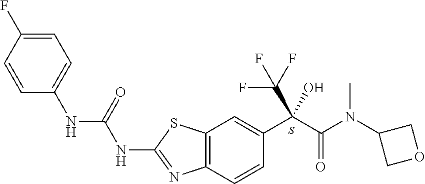 |
| 129 | 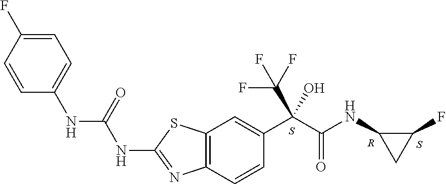 |
| 130 | 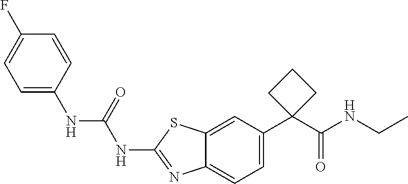 [SMILES: CCNC(=O)C1(c2ccc3nc(NC(=O)Nc4ccc(F)cc4)sc3c2)CCC1] |
| 131 | 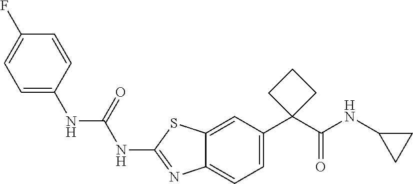 [SMILES: O=C(Nc1ccc(F)cc1)Nc1nc2ccc(C3(C(=O)NC4CC4)CCC3)cc2s1] |
| 132 | 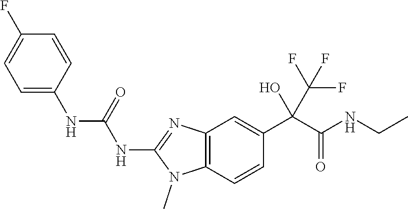 [SMILES: CCNC(=O)C(O)(c1ccc2c(c1)nc(NC(=O)Nc1ccc(F)cc1)n2C)C(F)(F)F] |
| 133 | 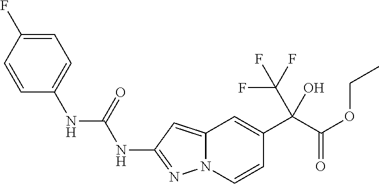 [SMILES: CCOC(=O)C(O)(c1ccn2nc(NC(=O)Nc3ccc(F)cc3)cc2c1)C(F)(F)F] |
| 134 | 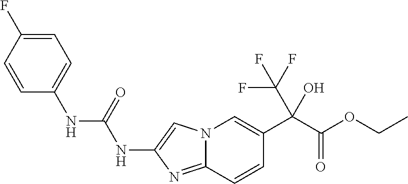 [SMILES: CCOC(=O)C(O)(c1ccc2nc(NC(=O)Nc3ccc(F)cc3)cn2c1)C(F)(F)F] |
| 135 | 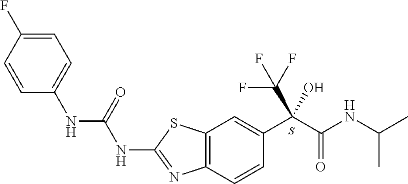 |
| 136 | 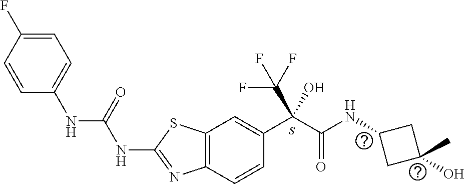 |
| 137 | 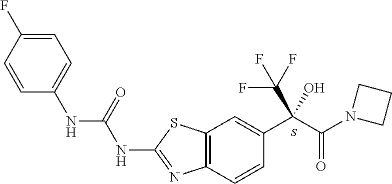 |
| 138 | 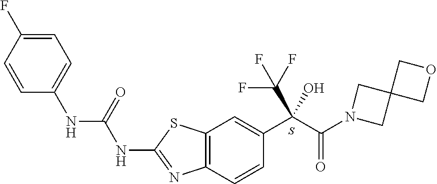 |
| 139 | 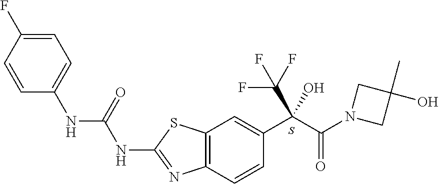 |
| 140 | 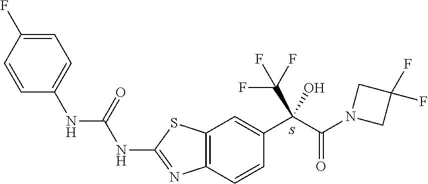 |
| 141 | 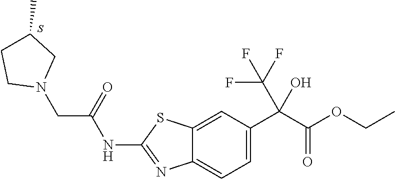 |
| 142 | 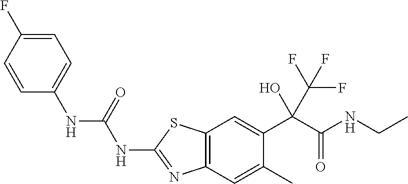 [SMILES: CCNC(=O)C(O)(c1cc2sc(NC(=O)Nc3ccc(F)cc3)nc2cc1C)C(F)(F)F] |
| 143 | 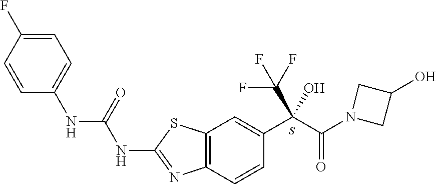 |
| 144 | 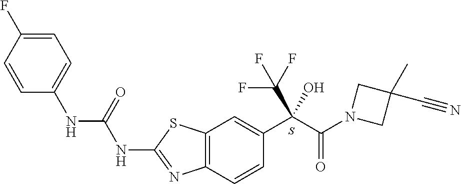 |
| 145 | 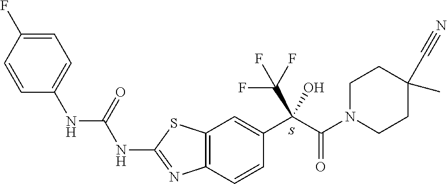 |
| 146 | 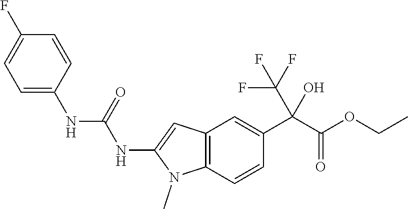 [SMILES: CCOC(=O)C(O)(c1ccc2c(c1)cc(NC(=O)Nc1ccc(F)cc1)n2C)C(F)(F)F] |
| 147 | 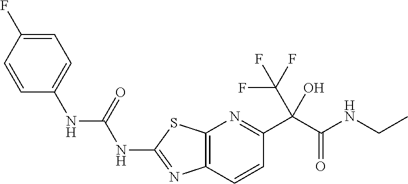 [SMILES: CCNC(=O)C(O)(c1ccc2nc(NC(=O)Nc3ccc(F)cc3)sc2n1)C(F)(F)F] |
| 148 | 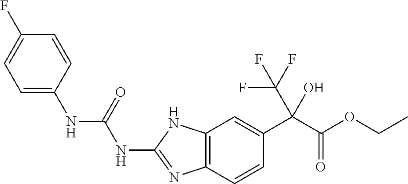 [SMILES: CCOC(=O)C(O)(c1ccc2nc(NC(=O)Nc3ccc(F)cc3)[nH]c2c1)C(F)(F)F] |
| 149 | 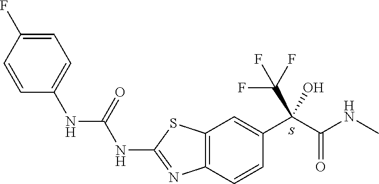 |
| 150 | 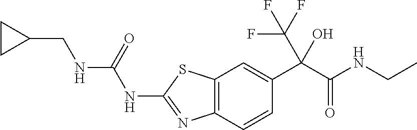 [SMILES: CCNC(=O)C(O)(c1ccc2nc(NC(=O)NCC3CC3)sc2c1)C(F)(F)F] |
| 151 | 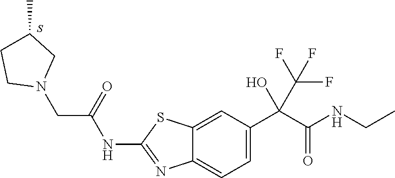 |
| 152 | 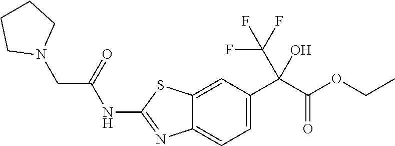 [SMILES: CCOC(=O)C(O)(c1ccc2nc(NC(=O)CN3CCCC3)sc2c1)C(F)(F)F] |
| 153 | 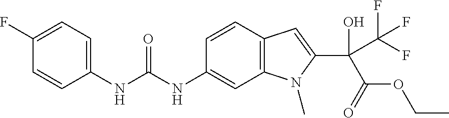 [SMILES: CCOC(=O)C(O)(c1cc2ccc(NC(=O)Nc3ccc(F)cc3)cc2n1C)C(F)(F)F] |
| 154 | 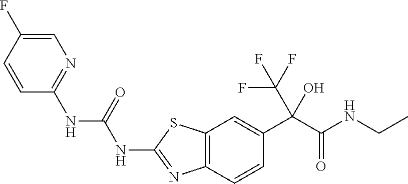 [SMILES: CCNC(=O)C(O)(c1ccc2nc(NC(=O)Nc3ccc(F)cn3)sc2c1)C(F)(F)F] |
| 155 | 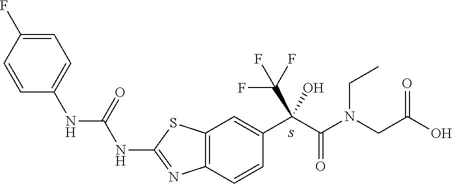 |
| 156 | 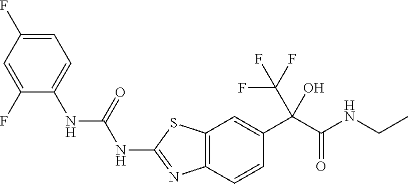 [SMILES: CCNC(=O)C(O)(c1ccc2nc(NC(=O)Nc3ccc(F)cc3F)sc2c1)C(F)(F)F] |
| 157 | 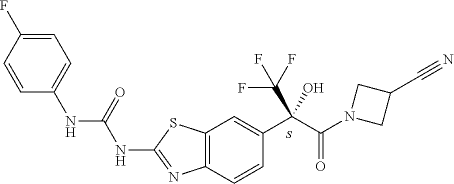 |
| 158 | 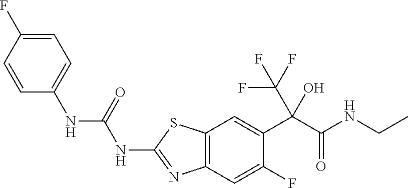 [SMILES: CCNC(=O)C(O)(c1cc2sc(NC(=O)Nc3ccc(F)cc3)nc2cc1F)C(F)(F)F] |
| 159 | 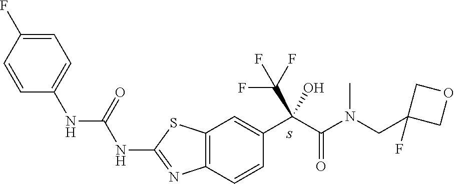 |
| 160 | 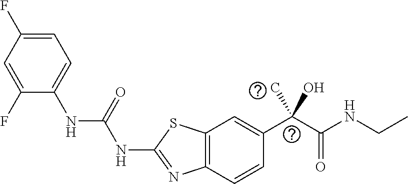 [SMILES: CCNC(=O)[C@](C)(O)c1ccc2nc(NC(=O)Nc3ccc(F)cc3F)sc2c1] |
| 161 | 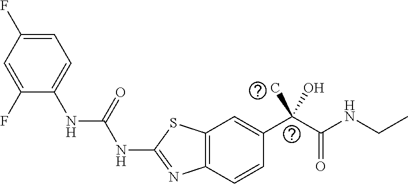 [SMILES: CCNC(=O)[C@@](C)(O)c1ccc2nc(NC(=O)Nc3ccc(F)cc3F)sc2c1] |
| 162 | 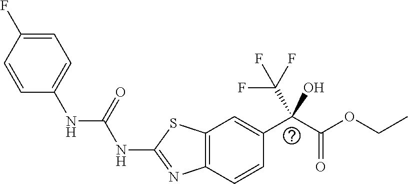 [SMILES: CCOC(=O)[C@](O)(c1ccc2nc(NC(=O)Nc3ccc(F)cc3)sc2c1)C(F)(F)F] |
| 164 | 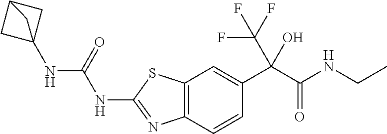 [SMILES: CCNC(=O)C(O)(c1ccc2nc(NC(=O)NC34CC(C3)C4)sc2c1)C(F)(F)F] |
| 165 | 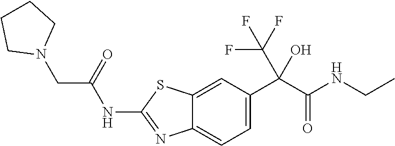 [SMILES: CCNC(=O)C(O)(c1ccc2nc(NC(=O)CN3CCCC3)sc2c1)C(F)(F)F] |
| 166 | 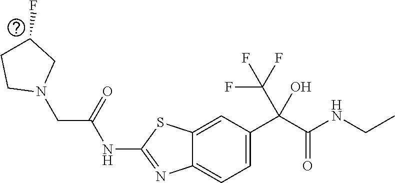 [SMILES: CCNC(=O)C(O)(c1ccc2nc(NC(=O)CN3CC[C@H](F)C3)sc2c1)C(F)(F)F] |
| 167 | 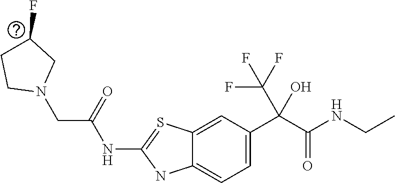 |
| 168 | 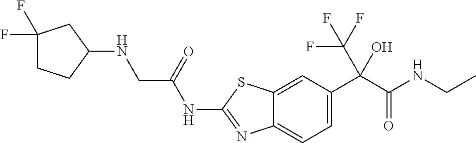 [SMILES: CCNC(=O)C(O)(c1ccc2nc(NC(=O)CNC3CCC(F)(F)C3)sc2c1)C(F)(F)F] |
| 169 | 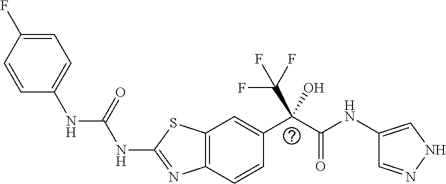 [SMILES: O=C(Nc1ccc(F)cc1)Nc1nc2ccc([C@](O)(C(=O)Nc3cn[nH]c3)C(F)(F)F)cc2s1] |
| 170 | 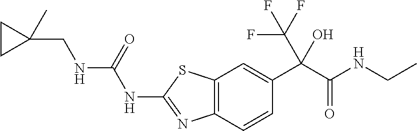 [SMILES: CCNC(=O)C(O)(c1ccc2nc(NC(=O)NCC3(C)CC3)sc2c1)C(F)(F)F] |
| 171 | 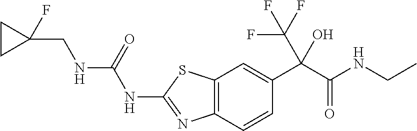 [SMILES: CCNC(=O)C(O)(c1ccc2nc(NC(=O)NCC3(F)CC3)sc2c1)C(F)(F)F] |
| 172 | 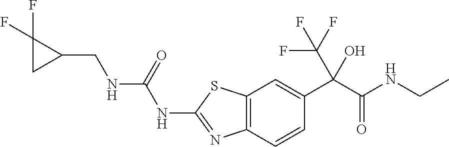 [SMILES: CCNC(=O)C(O)(c1ccc2nc(NC(=O)NCC3CC3(F)F)sc2c1)C(F)(F)F] |
| 173 | 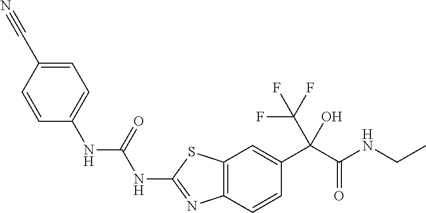 [SMILES: CCNC(=O)C(O)(c1ccc2nc(NC(=O)Nc3ccc(C#N)cc3)sc2c1)C(F)(F)F] |
| 174 | 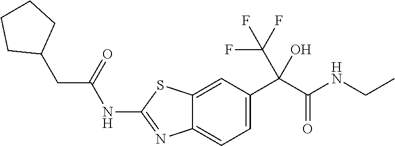 [SMILES: CCNC(=O)C(O)(c1ccc2nc(NC(=O)CC3CCCC3)sc2c1)C(F)(F)F] |
| 175 | 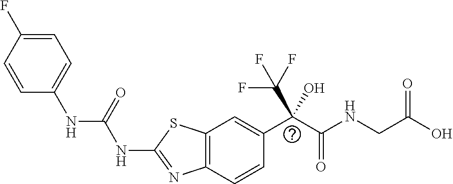 [SMILES: O=C(O)CNC(=O)[C@@](O)(c1ccc2nc(NC(=O)Nc3ccc(F)cc3)sc2c1)C(F)(F)F] |
| 176 | 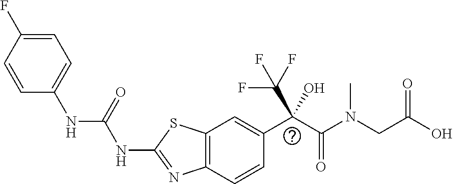 [SMILES: CN(CC(=O)O)C(=O)[C@@](O)(c1ccc2nc(NC(=O)Nc3ccc(F)cc3)sc2c1)C(F)(F)F] |
| 177 | 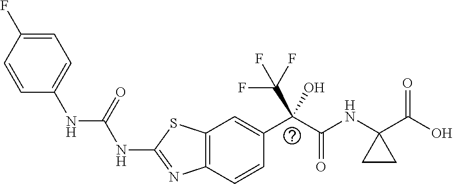 [SMILES: O=C(Nc1ccc(F)cc1)Nc1nc2ccc([C@](O)(C(=O)NC3(C(=O)O)CC3)C(F)(F)F)cc2s1] |
| 178 | 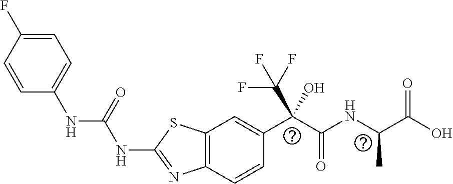 [SMILES: C[C@@H](NC(=O)[C@@](O)(c1ccc2nc(NC(=O)Nc3ccc(F)cc3)sc2c1)C(F)(F)F)C(=O)O] |
| 179 | 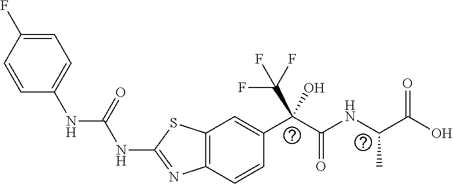 [SMILES: C[C@H](NC(=O)[C@@](O)(c1ccc2nc(NC(=O)Nc3ccc(F)cc3)sc2c1)C(F)(F)F)C(=O)O] |
| 180 | 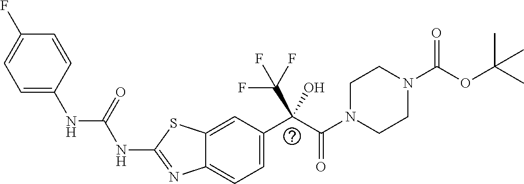 [SMILES: CC(C)(C)OC(=O)N1CCN(C(=O)[C@@](O)(c2ccc3nc(NC(=O)Nc4ccc(F)cc4)sc3c2)C(F)(F)F)CC1] |
| 181 | 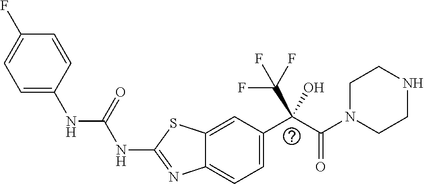 [SMILES: O=C(Nc1ccc(F)cc1)Nc1nc2ccc([C@](O)(C(=O)N3CCNCC3)C(F)(F)F)cc2s1] |
| 182 | 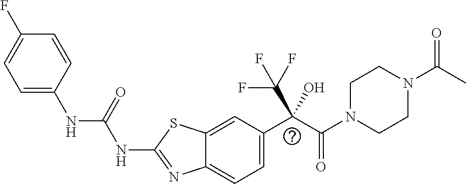 [SMILES: CC(=O)N1CCN(C(=O)[C@@](O)(c2ccc3nc(NC(=O)Nc4ccc(F)cc4)sc3c2)C(F)(F)F)CC1] |
| 183 | 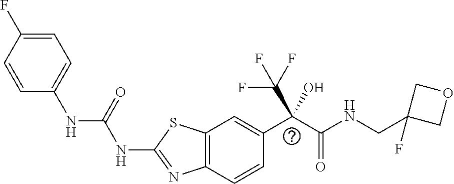 [SMILES: O=C(Nc1ccc(F)cc1)Nc1nc2ccc([C@](O)(C(=O)NCC3(F)COC3)C(F)(F)F)cc2s1] |
| 184 | 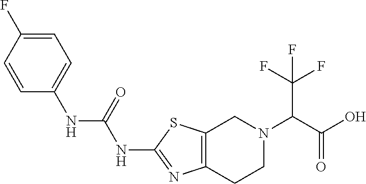 [SMILES: O=C(Nc1ccc(F)cc1)Nc1nc2c(s1)CN(C(C(=O)O)C(F)(F)F)CC2] |
| 185 | 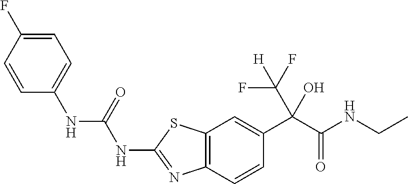 [SMILES: CCNC(=O)C(O)(c1ccc2nc(NC(=O)Nc3ccc(F)cc3)sc2c1)C(F)F] |
| 186 | 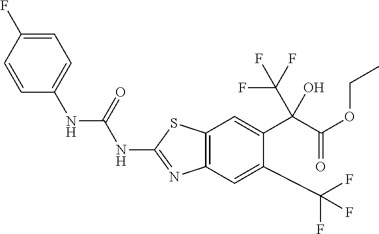 [SMILES: CCOC(=O)C(O)(c1cc2sc(NC(=O)Nc3ccc(F)cc3)nc2cc1C(F)(F)F)C(F)(F)F] |
| 187 | 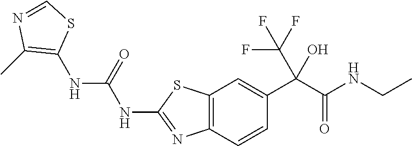 [SMILES: CCNC(=O)C(O)(c1ccc2nc(NC(=O)Nc3scnc3C)sc2c1)C(F)(F)F] |
| 188 | 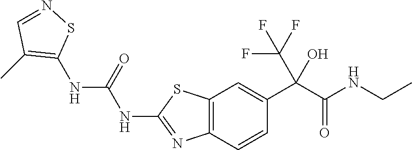 [SMILES: CCNC(=O)C(O)(c1ccc2nc(NC(=O)Nc3sncc3C)sc2c1)C(F)(F)F] |
| 189 | 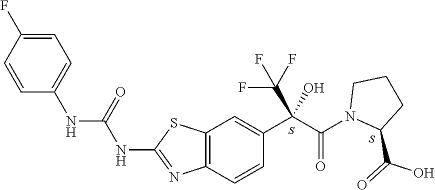 |
| 190 | 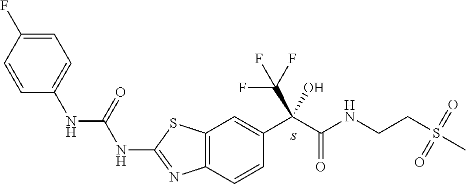 |
| 191 | 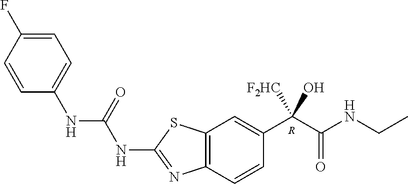 |
| 192 | 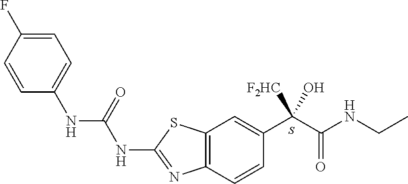 |
| 193 | 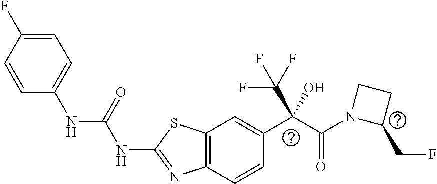 [SMILES: O=C(Nc1ccc(F)cc1)Nc1nc2ccc([C@](O)(C(=O)N3CC[C@H]3CF)C(F)(F)F)cc2s1] |
| 194 | 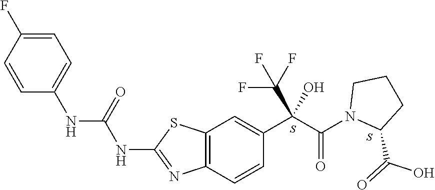 |
| 195 | 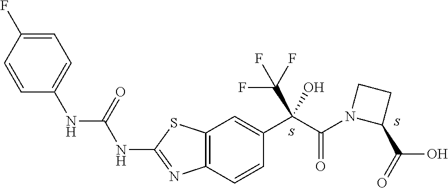 |
| 196 | 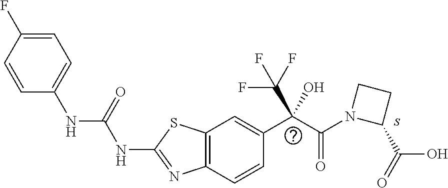 |
| 197 | 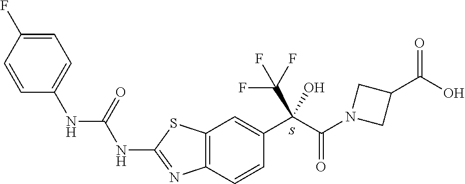 |
| 198 | 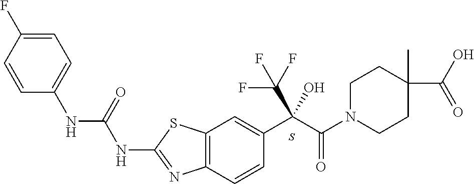 |
| 199 | 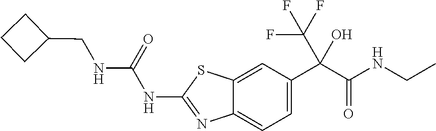 [SMILES: CCNC(=O)C(O)(c1ccc2nc(NC(=O)NCC3CCC3)sc2c1)C(F)(F)F] |
| 200 | 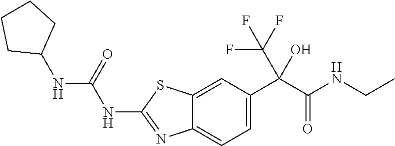 [SMILES: CCNC(=O)C(O)(c1ccc2nc(NC(=O)NC3CCCC3)sc2c1)C(F)(F)F] |
| 201 | 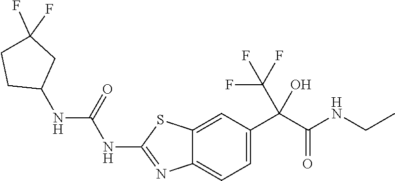 [SMILES: CCNC(=O)C(O)(c1ccc2nc(NC(=O)NC3CCC(F)(F)C3)sc2c1)C(F)(F)F] |
| 202 | 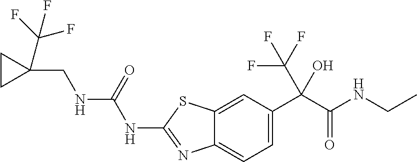 [SMILES: CCNC(=O)C(O)(c1ccc2nc(NC(=O)NCC3(C(F)(F)F)CC3)sc2c1)C(F)(F)F] |
| 203 | 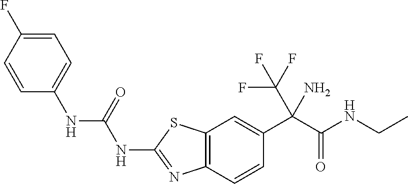 [SMILES: CCNC(=O)C(N)(c1ccc2nc(NC(=O)Nc3ccc(F)cc3)sc2c1)C(F)(F)F] |
| 204 | 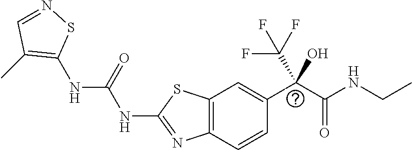 [SMILES: CCNC(=O)[C@](O)(c1ccc2nc(NC(=O)Nc3sncc3C)sc2c1)C(F)(F)F] |
| 205 | 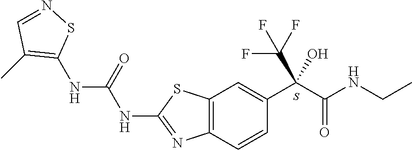 |
| 206 | 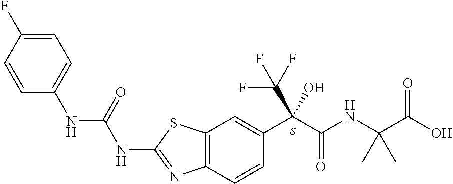 |
| 207 | 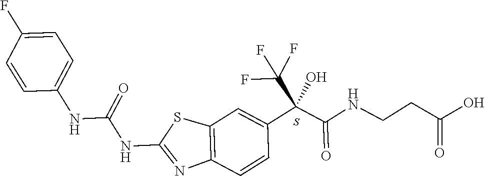 |
| 208 | 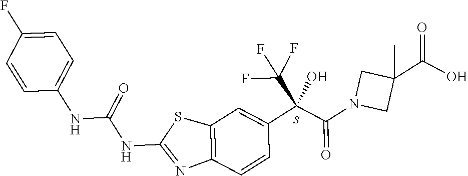 |
| 209 | 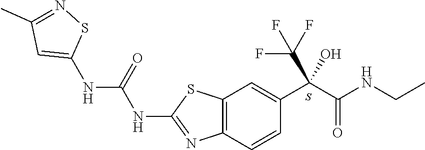 |
| 210 | 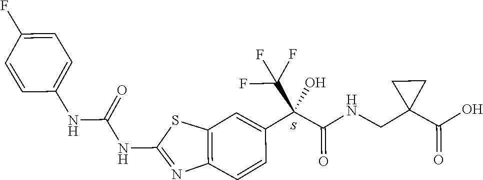 |
| 211 | 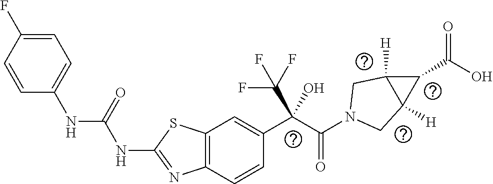 [SMILES: O=C(Nc1ccc(F)cc1)Nc1nc2ccc([C@](O)(C(=O)N3C[C@@H]4[C@H](C3)[C@H]4C(=O)O)C(F)(F)F)cc2s1] |

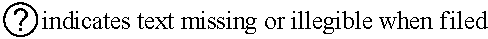

or a pharmaceutically acceptable salt thereof.

119. A compound having a structure selected from the following table:

| # | Structure |
|---|---|
| 212 | 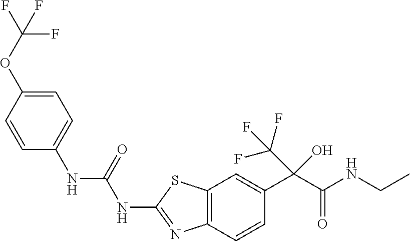 [SMILES: CCNC(=O)C(O)(c1ccc2nc(NC(=O)Nc3ccc(OC(F)(F)F)cc3)sc2c1)C(F)(F)F] |
| 213 | 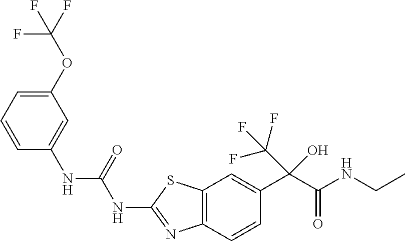 [SMILES: CCNC(=O)C(O)(c1ccc2nc(NC(=O)Nc3cccc(OC(F)(F)F)c3)sc2c1)C(F)(F)F] |
| 214 | 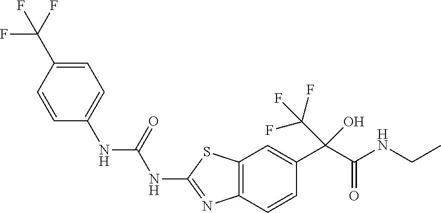 [SMILES: CCNC(=O)C(O)(c1ccc2nc(NC(=O)Nc3ccc(C(F)(F)F)cc3)sc2c1)C(F)(F)F] |
| 215 | 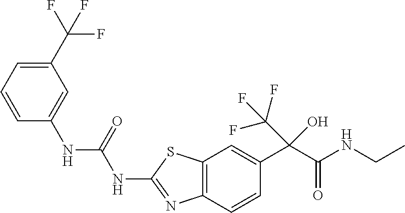 [SMILES: CCNC(=O)C(O)(c1ccc2nc(NC(=O)Nc3cccc(C(F)(F)F)c3)sc2c1)C(F)(F)F] |
| 216 | 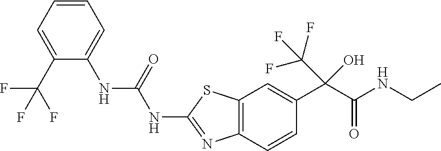 [SMILES: CCNC(=O)C(O)(c1ccc2nc(NC(=O)Nc3ccccc3C(F)(F)F)sc2c1)C(F)(F)F] |
| 217 | 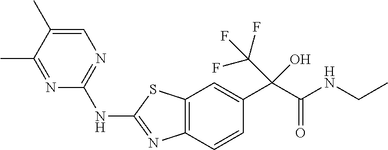 [SMILES: CCNC(=O)C(O)(c1ccc2nc(Nc3ncc(C)c(C)n3)sc2c1)C(F)(F)F] |
| 218 | 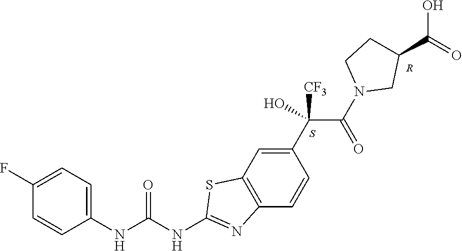 |
| 219 | 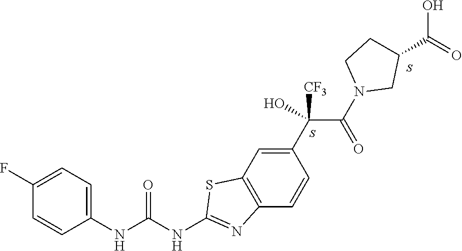 |
| 220 | 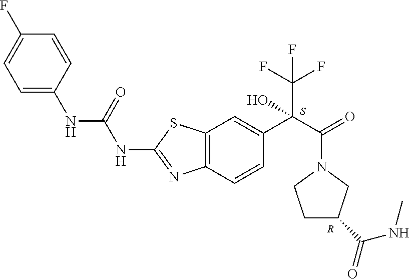 |
| 221 | 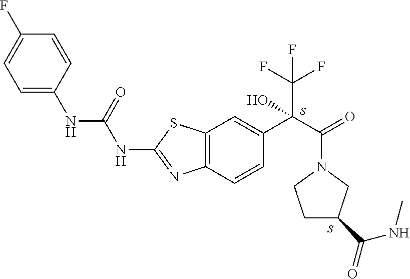 |
| 222 | 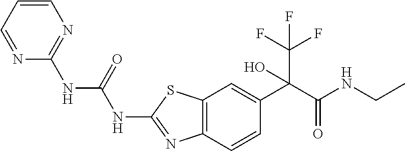 [SMILES: CCNC(=O)C(O)(c1ccc2nc(NC(=O)Nc3ncccn3)sc2c1)C(F)(F)F] |
| 223 | 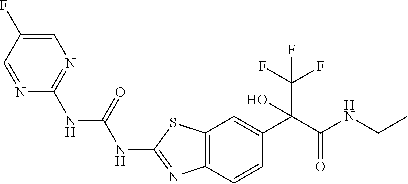 [SMILES: CCNC(=O)C(O)(c1ccc2nc(NC(=O)Nc3ncc(F)cn3)sc2c1)C(F)(F)F] |
| 224 | 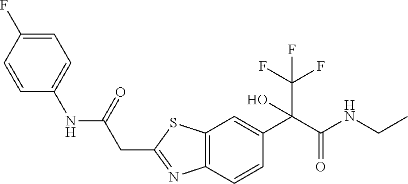 [SMILES: CCNC(=O)C(O)(c1ccc2nc(CC(=O)Nc3ccc(F)cc3)sc2c1)C(F)(F)F] |
| 225 | 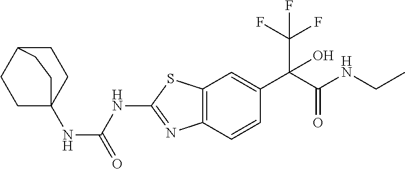 [SMILES: CCNC(=O)C(O)(c1ccc2nc(NC(=O)NC34CCC(CC3)CC4)sc2c1)C(F)(F)F] |
| 226 | 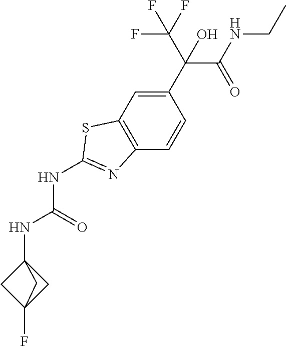 [SMILES: CCNC(=O)C(O)(c1ccc2nc(NC(=O)NC34CC(F)(C3)C4)sc2c1)C(F)(F)F] |
| 227 | 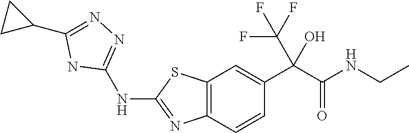 [SMILES: CCNC(=O)C(O)(c1ccc2nc(Nc3nnc(C4CC4)[nH]3)sc2c1)C(F)(F)F] |
| 228 | 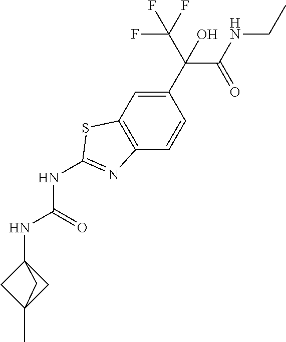 [SMILES: CCNC(=O)C(O)(c1ccc2nc(NC(=O)NC34CC(C)(C3)C4)sc2c1)C(F)(F)F] |
| 229 | 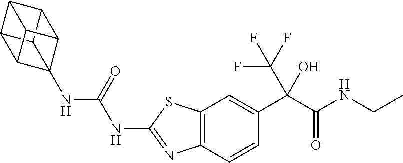 [SMILES: CCNC(=O)C(O)(c1ccc2nc(NC(=O)NC34C5C6C7C5C3C7C64)sc2c1)C(F)(F)F] |
| 230 | 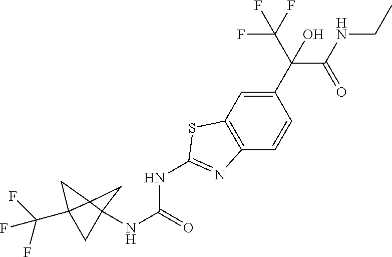 [SMILES: CCNC(=O)C(O)(c1ccc2nc(NC(=O)NC34CC5(C(F)(F)F)CC35C4)sc2c1)C(F)(F)F] |
| 231 | 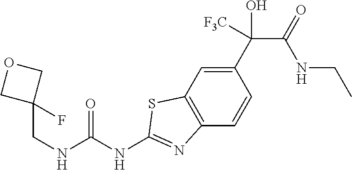 [SMILES: CCNC(=O)C(O)(c1ccc2nc(NC(=O)NCC3(F)COC3)sc2c1)C(F)(F)F] |
| 232 | 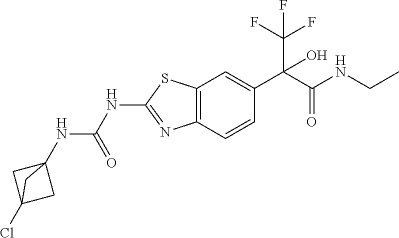 [SMILES: CCNC(=O)C(O)(c1ccc2nc(NC(=O)NC34CC(Cl)(C3)C4)sc2c1)C(F)(F)F] |
| 233 | 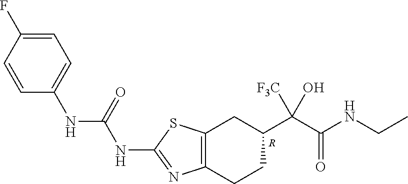 |
| 234 | 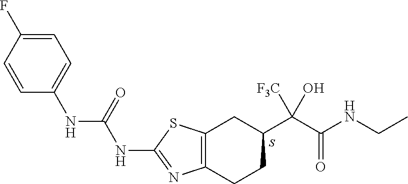 |
| 235 | 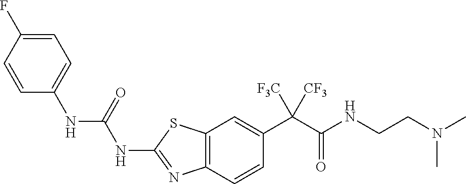 [SMILES: CN(C)CCNC(=O)C(c1ccc2nc(NC(=O)Nc3ccc(F)cc3)sc2c1)(C(F)(F)F)C(F)(F)F] |
| 236 | 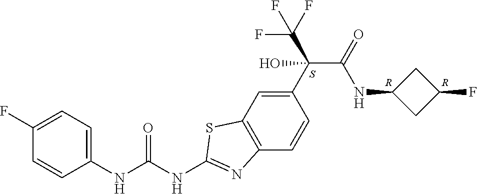 |
| 237 | 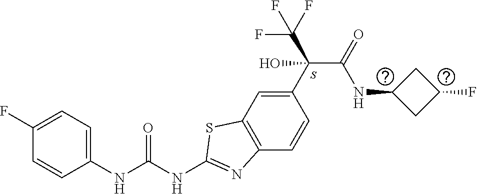 |
| 238 | 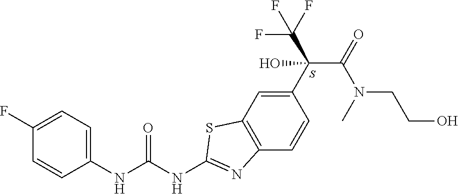 |
| 239 | 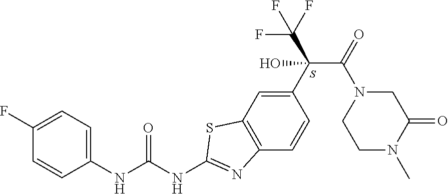 |
| 240 | 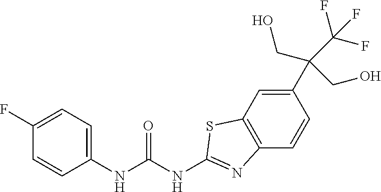 [SMILES: O=C(Nc1ccc(F)cc1)Nc1nc2ccc(C(CO)(CO)C(F)(F)F)cc2s1] |
| 241 | 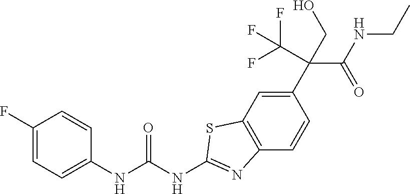 [SMILES: CCNC(=O)C(CO)(c1ccc2nc(NC(=O)Nc3ccc(F)cc3)sc2c1)C(F)(F)F] |
| 242 | 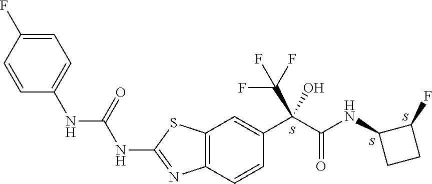 |
| 243 | 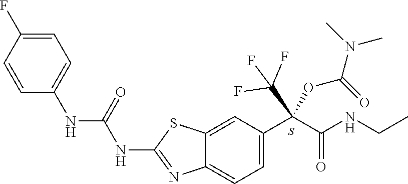 |
| 244 | 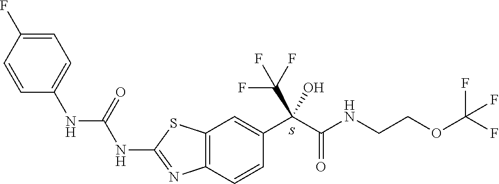 |
| 245 | 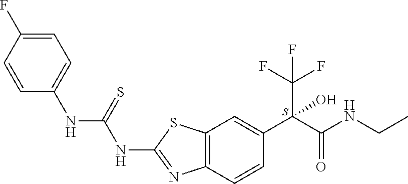 |
| 246 | 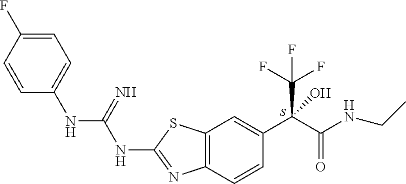 |
| 247 | 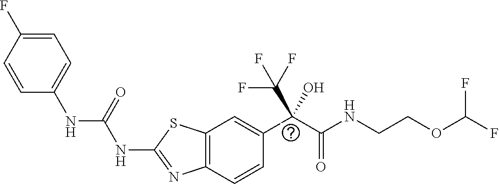 [SMILES: O=C(Nc1ccc(F)cc1)Nc1nc2ccc([C@](O)(C(=O)NCCOC(F)F)C(F)(F)F)cc2s1] |
| 248 | 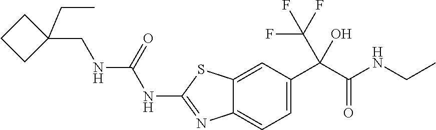 [SMILES: CCNC(=O)C(O)(c1ccc2nc(NC(=O)NCC3(CC)CCC3)sc2c1)C(F)(F)F] |
| 249 | 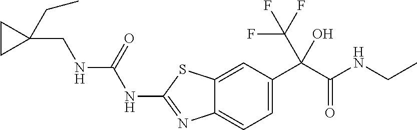 [SMILES: CCNC(=O)C(O)(c1ccc2nc(NC(=O)NCC3(CC)CC3)sc2c1)C(F)(F)F] |
| 250 | 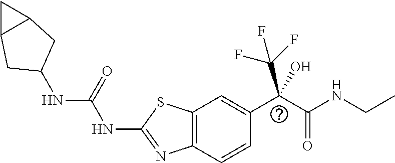 [SMILES: CCNC(=O)[C@@](O)(c1ccc2nc(NC(=O)NC3CC4CC4C3)sc2c1)C(F)(F)F] |
| 251 | 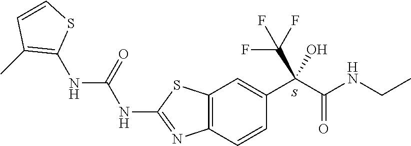 |
| 252 | 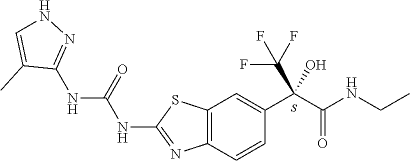 |
| 253 | 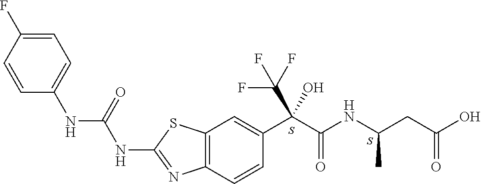 |
| 254 | 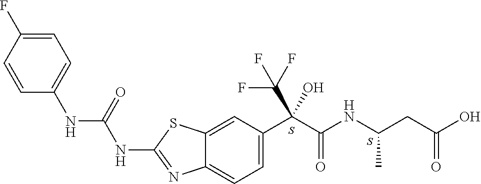 |
| 255 | 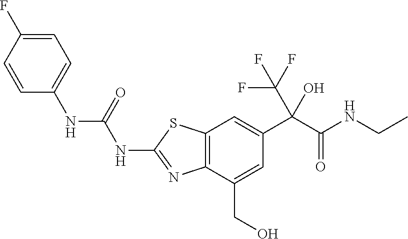 [SMILES: CCNC(=O)C(O)(c1cc(CO)c2nc(NC(=O)Nc3ccc(F)cc3)sc2c1)C(F)(F)F] |
| 256 | 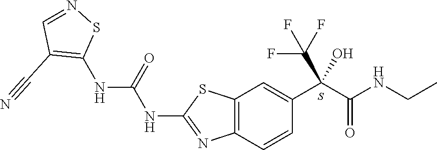 |
| 257 | 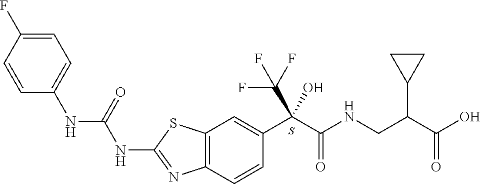 |
| 258 | 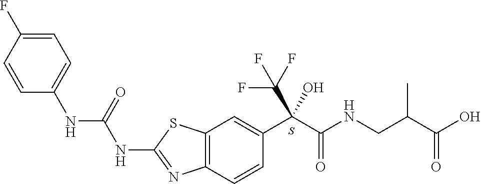 |
| 259 | 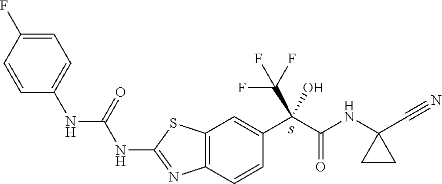 |
| 260 | 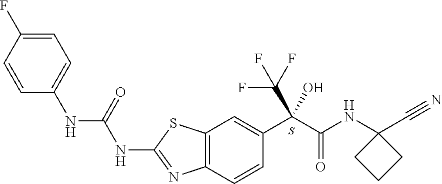 |
| 261 | 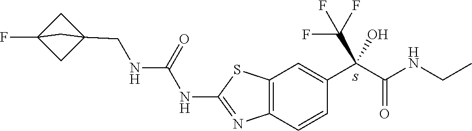 |
| 262 | 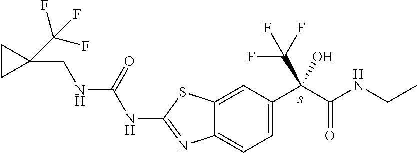 |
| 263 | 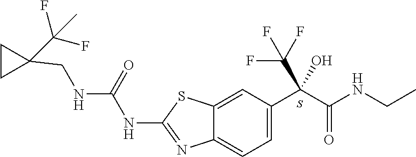 |
| 264 | 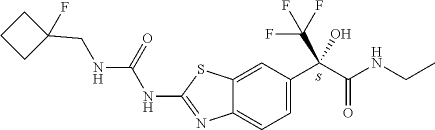 |
| 265 | 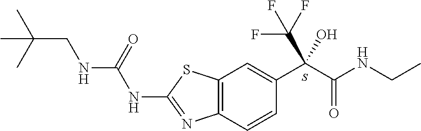 |
| 266 | 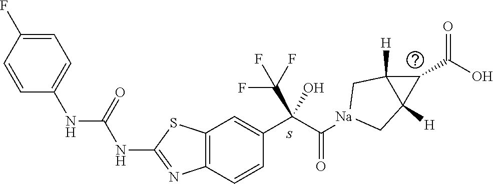 |
| 267 | 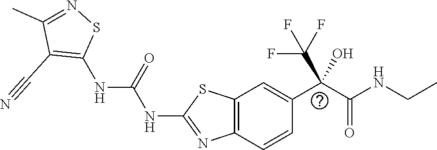 [SMILES: CCNC(=O)[C@@](O)(c1ccc2nc(NC(=O)Nc3snc(C)c3C#N)sc2c1)C(F)(F)F] |
| 268 | 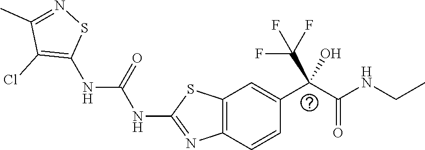 [SMILES: CCNC(=O)[C@@](O)(c1ccc2nc(NC(=O)Nc3snc(C)c3Cl)sc2c1)C(F)(F)F] |
| 269 | 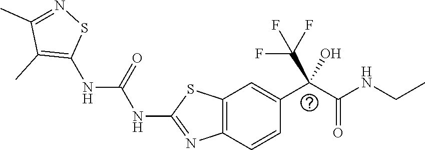 [SMILES: CCNC(=O)[C@@](O)(c1ccc2nc(NC(=O)Nc3snc(C)c3C)sc2c1)C(F)(F)F] |
| 270 | 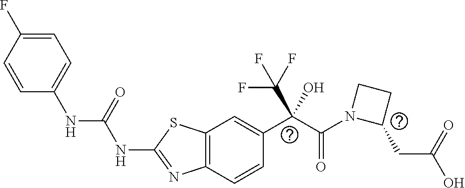 [SMILES: O=C(O)C[C@H]1CCN1C(=O)[C@@](O)(c1ccc2nc(NC(=O)Nc3ccc(F)cc3)sc2c1)C(F)(F)F] |
| 271 | 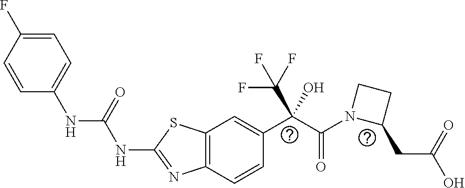 [SMILES: O=C(O)C[C@@H]1CCN1C(=O)[C@@](O)(c1ccc2nc(NC(=O)Nc3ccc(F)cc3)sc2c1)C(F)(F)F] |
| 272 | 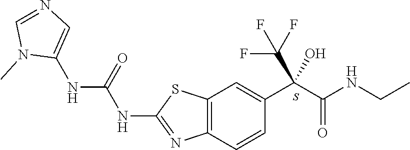 |
| 273 | 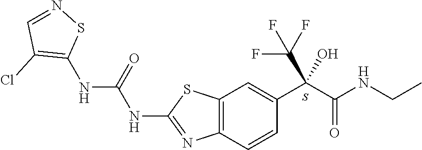 |
| 274 | 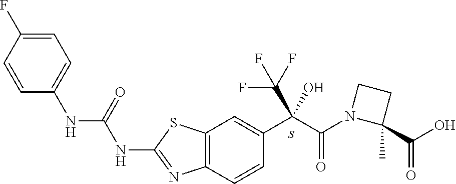 |
| 275 | 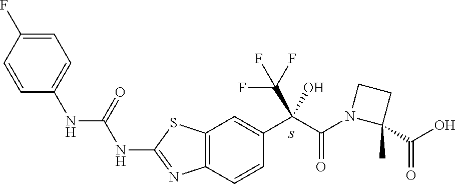 |
| 276 | 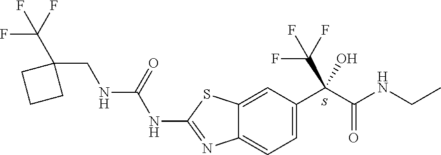 |
| 277 | 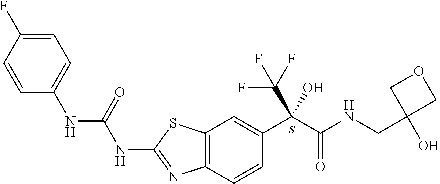 |
| 278 | 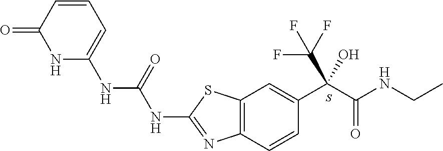 |
| 279 | 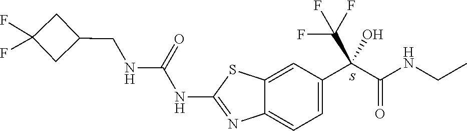 |
| 280 | 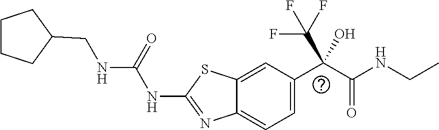 [SMILES: CCNC(=O)[C@@](O)(c1ccc2nc(NC(=O)NCC3CCCC3)sc2c1)C(F)(F)F] |
| 281 | 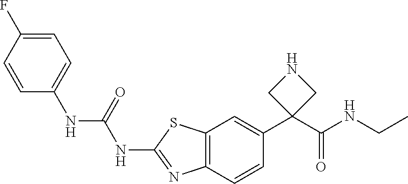 [SMILES: CCNC(=O)C1(c2ccc3nc(NC(=O)Nc4ccc(F)cc4)sc3c2)CNC1] |
| 282 | 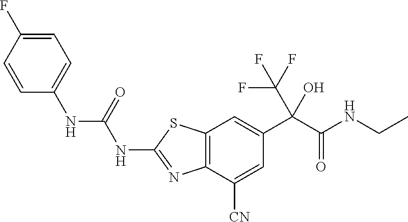 [SMILES: CCNC(=O)C(O)(c1cc(C#N)c2nc(NC(=O)Nc3ccc(F)cc3)sc2c1)C(F)(F)F] |
| 283 | 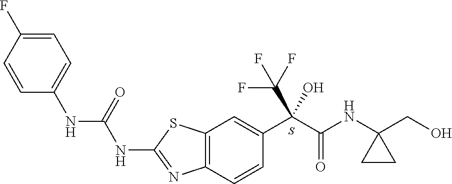 |
| 284 | 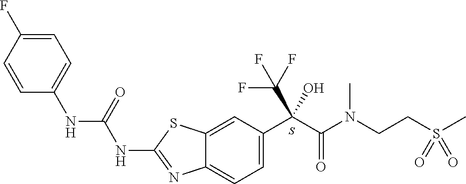 |
| 285 | 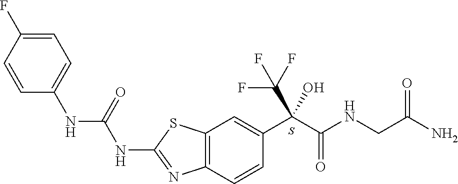 |
| 286 | 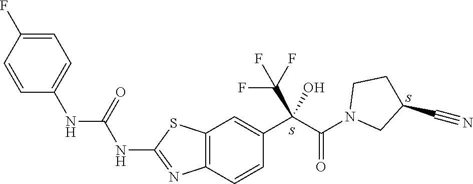 |
| 287 | 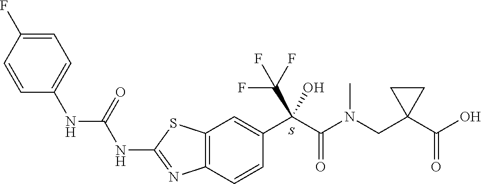 |
| 288 | 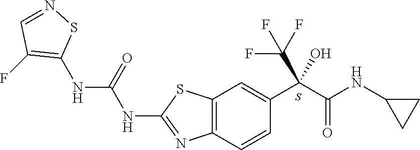 |
| 289 | 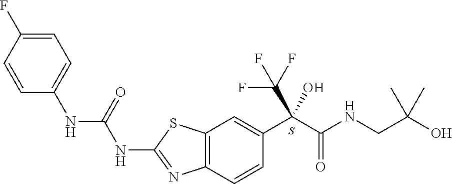 |
| 290 | 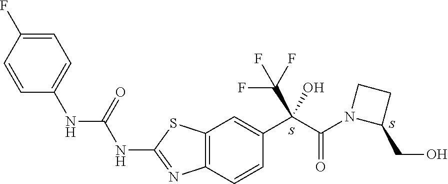 |
| 291 | 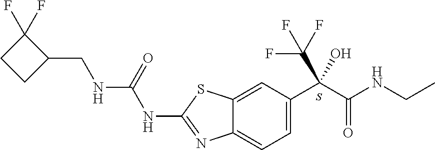 |
| 292 | 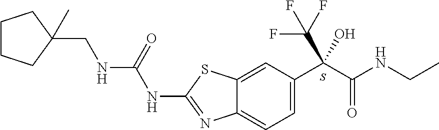 |
| 293 | 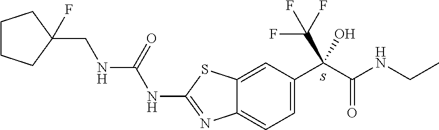 |
| 294 | 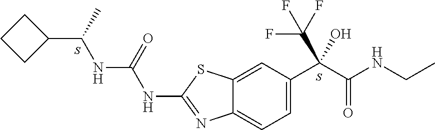 |
| 295 | 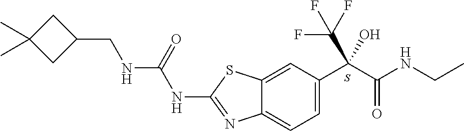 |
| 296 | 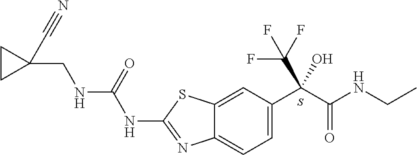 |
| 297 | 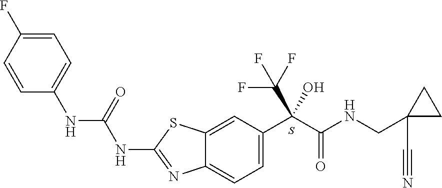 |
| 298 | 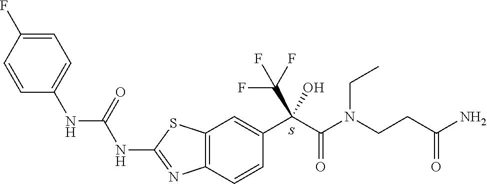 |
| 299 | 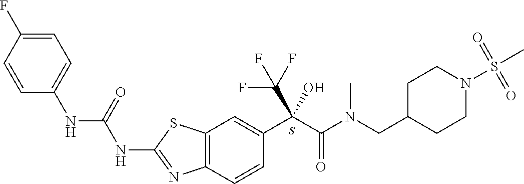 |
| 300 | 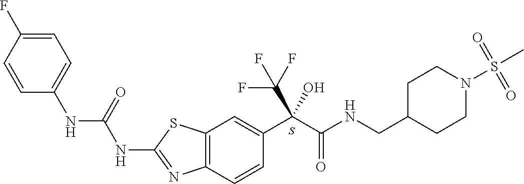 |
| 301 | 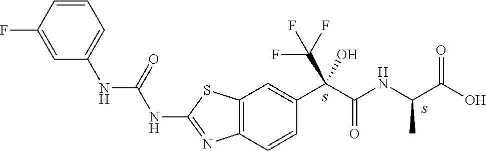 |
| 302 | 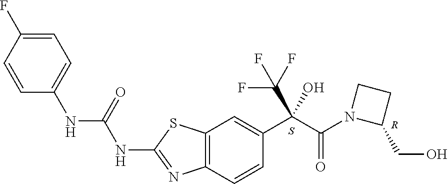 |
| 303 | 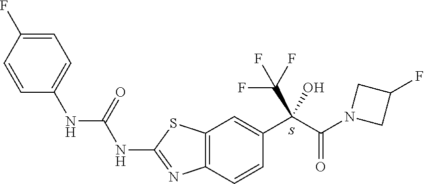 |
| 304 | 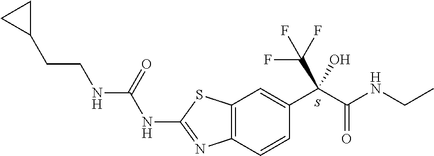 |
| 305 | 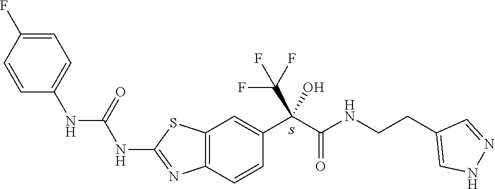 |
| 306 | 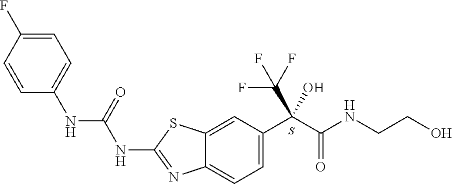 |
| 307 | 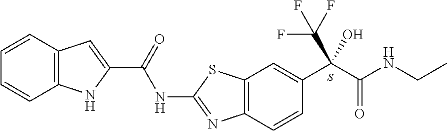 |
| 308 | 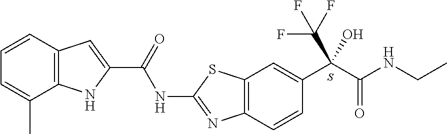 |
| 309 | 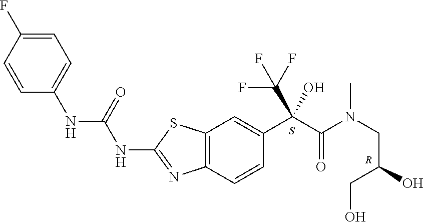 |
| 310 | 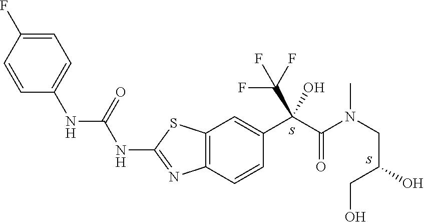 |
| 311 | 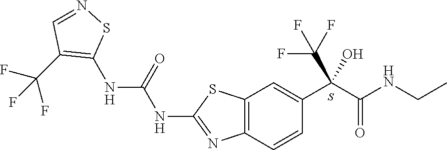 |
| 312 | 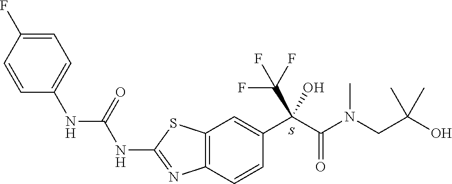 |
| 313 | 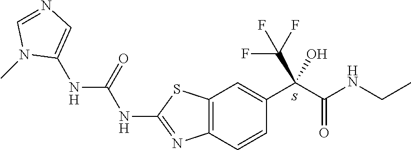 |
| 314 | 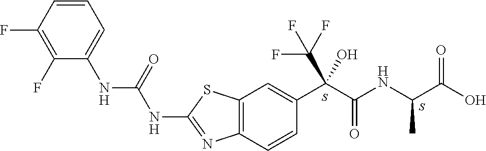 |
| 315 | 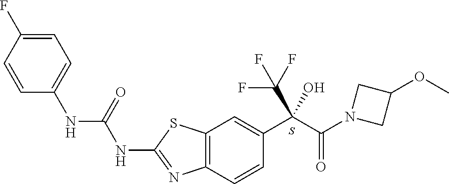 |
| 316 | 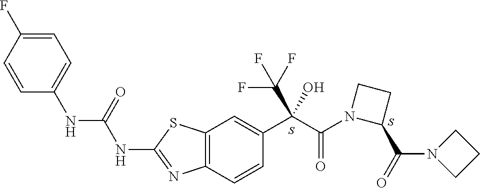 |
| 317 | 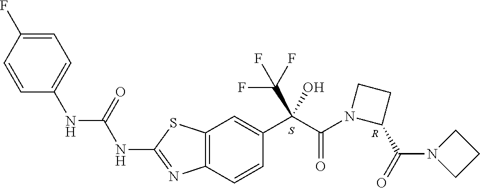 |
| 318 | 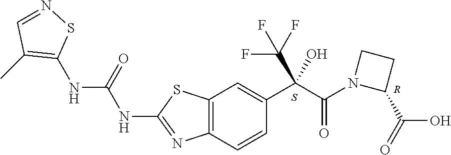 |
| 319 | 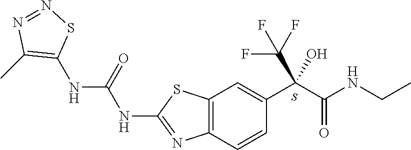 |
| 320 | 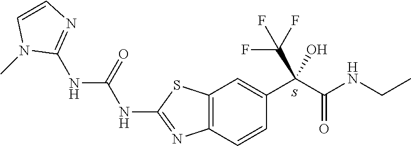 |
| 321 | 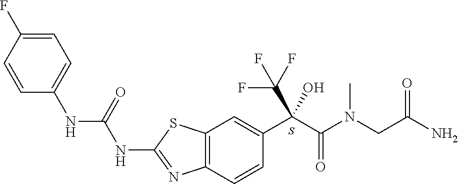 |
| 322 | 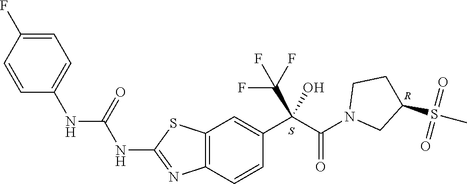 |
| 323 | 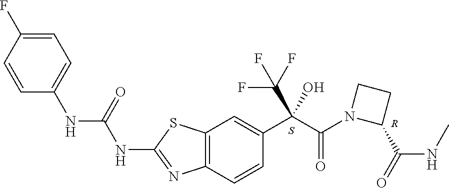 |
| 324 | 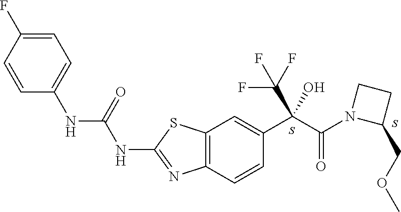 |
| 325 | 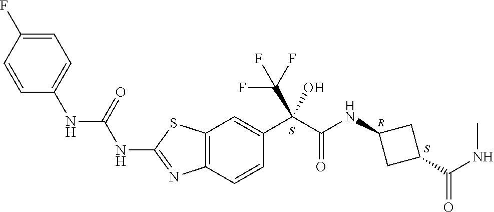 |
| 326 | 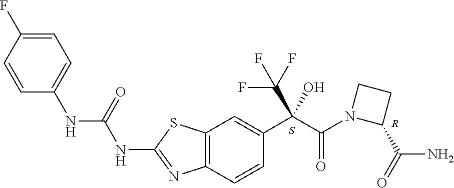 |
| 327 | 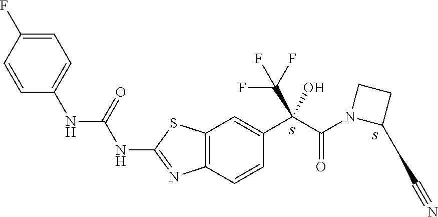 |
| 328 | 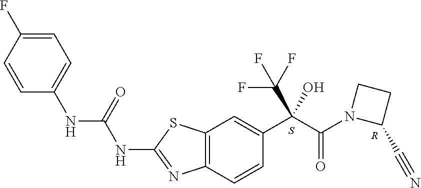 |
| 329 | 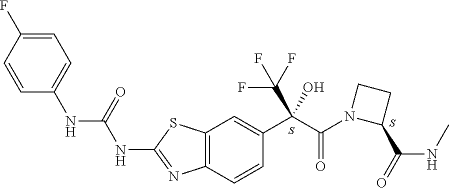 |
| 330 | 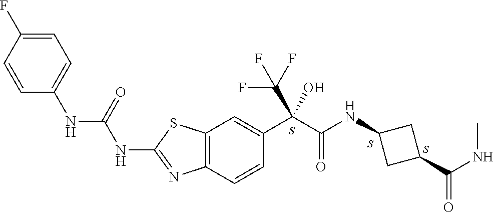 |
| 331 | 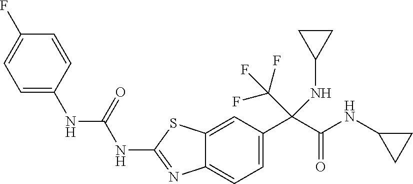 [SMILES: O=C(Nc1ccc(F)cc1)Nc1nc2ccc(C(NC3CC3)(C(=O)NC3CC3)C(F)(F)F)cc2s1] |
| 332 | 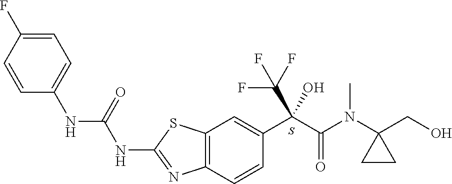 |
| 333 | 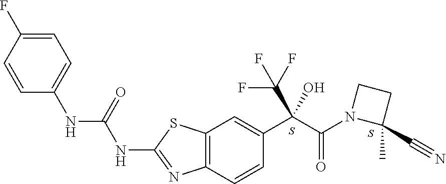 |
| 334 | 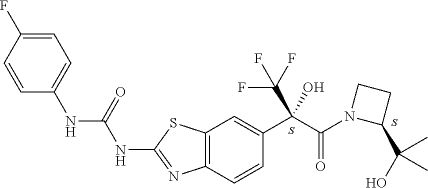 |
| 335 | 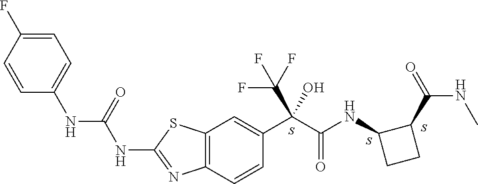 |
| 336 | 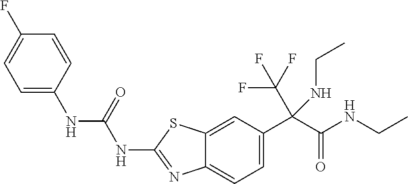 [SMILES: CCNC(=O)C(NCC)(c1ccc2nc(NC(=O)Nc3ccc(F)cc3)sc2c1)C(F)(F)F] |
| 337 | 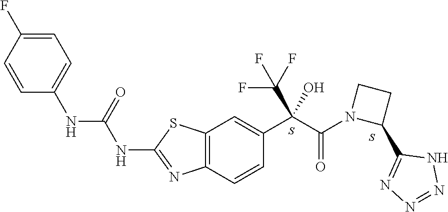 |
| 338 | 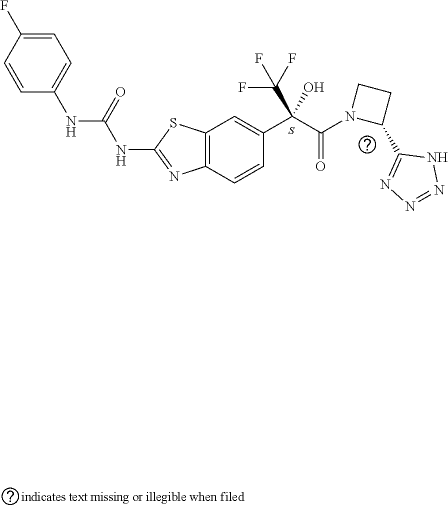 |
| 339 | 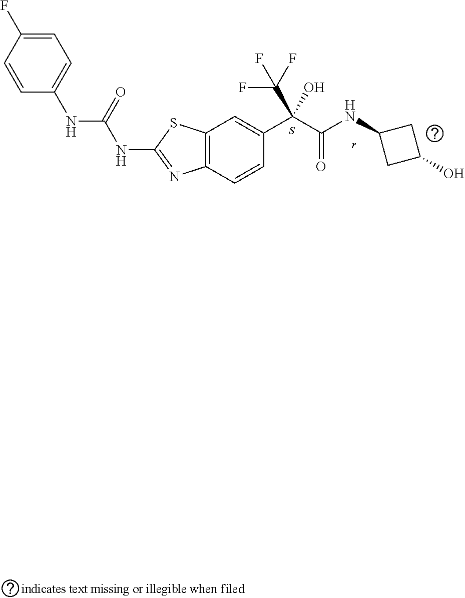 |
| 340 | 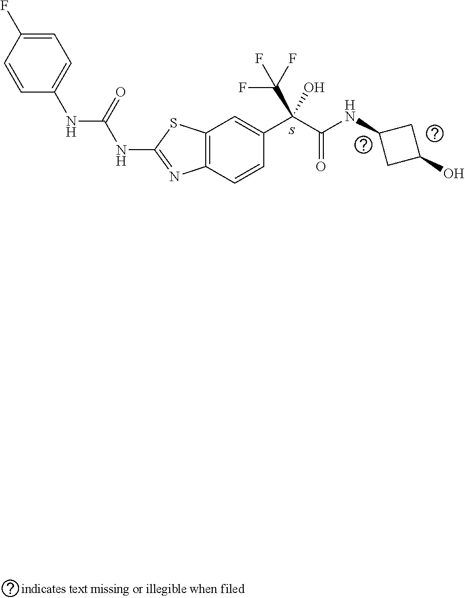 |
| 341 | 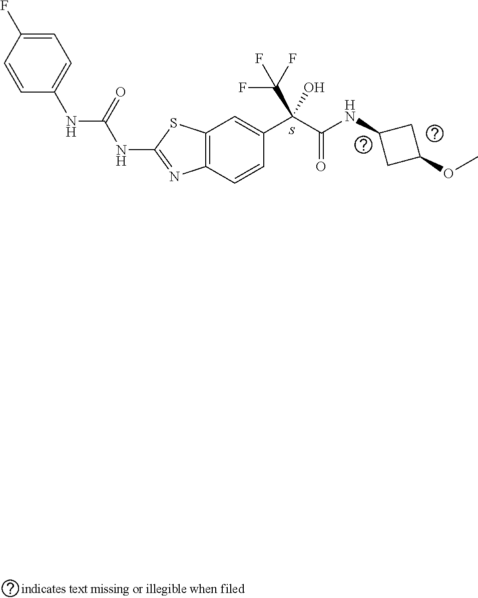 |
| 342 | 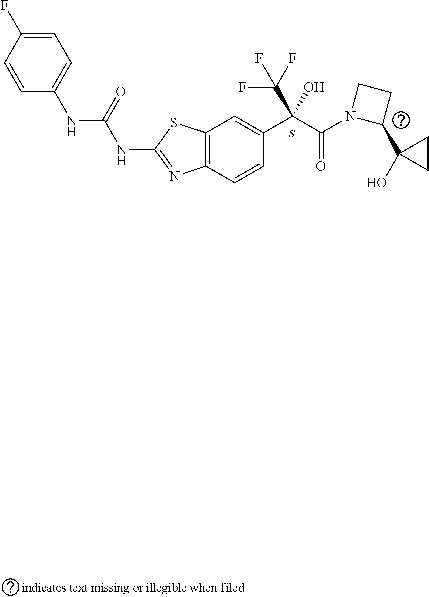 |
| 343 | 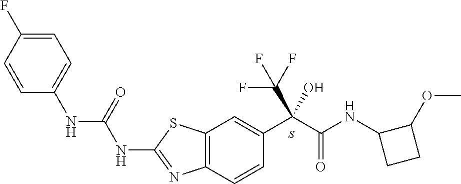 |
| 344 | 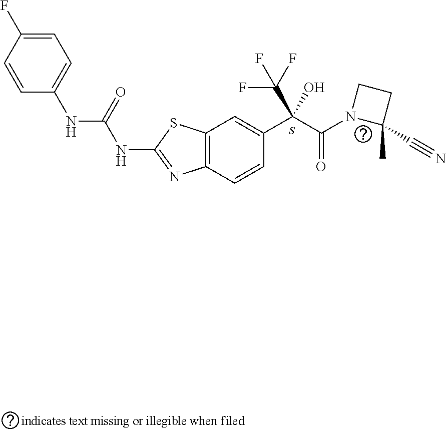 |
| 345 | 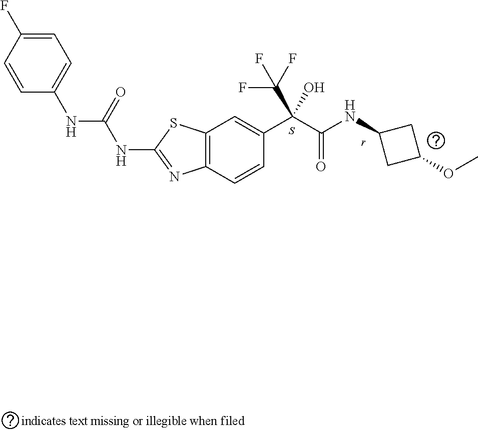 |
| 346 | 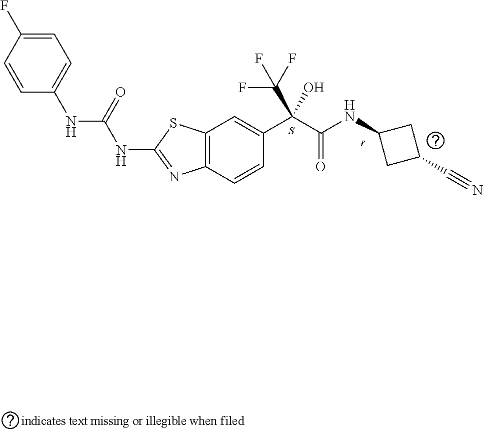 |
| 347 | 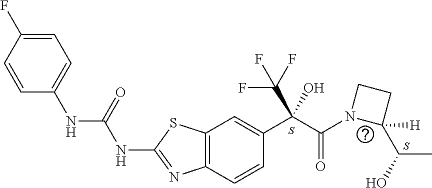 |
| 348 | 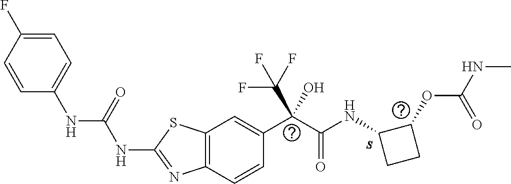 |
| 349 | 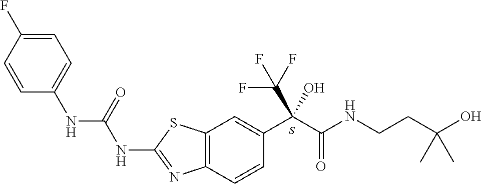 |
| 350 | 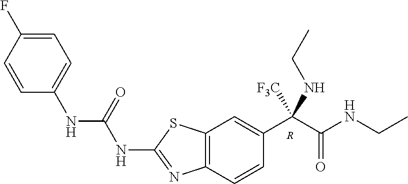 |
| 351 | 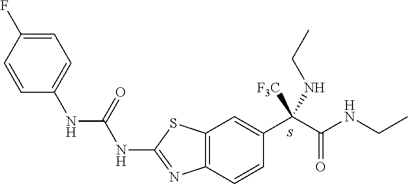 |
| 352 | 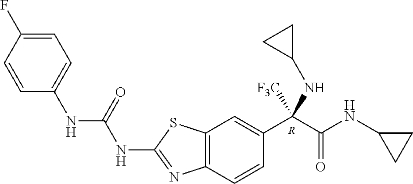 |
| 353 | 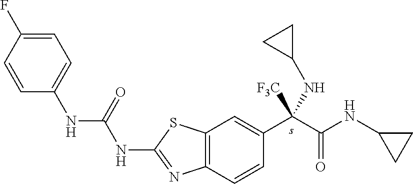 |
| 354 | 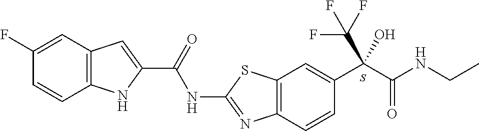 |
| 355 | 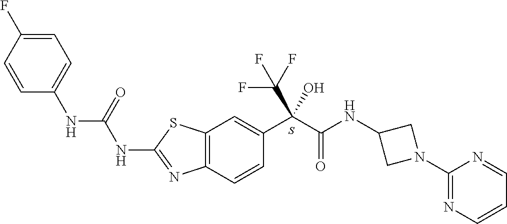 |
| 356 | 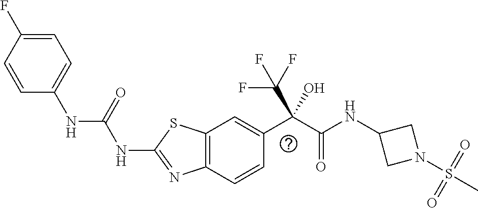 [SMILES: CS(=O)(=O)N1CC(NC(=O)[C@@](O)(c2ccc3nc(NC(=O)Nc4ccc(F)cc4)sc3c2)C(F)(F)F)C1] |
| 357 | 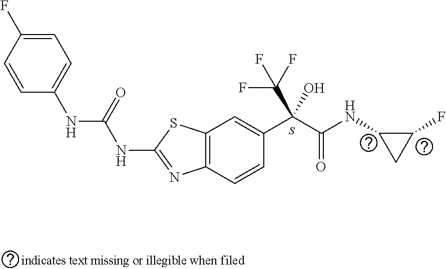 |
| 358 | 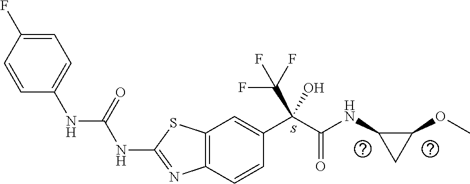 |
| 359 | 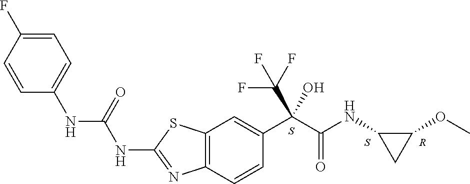 |
| 360 | 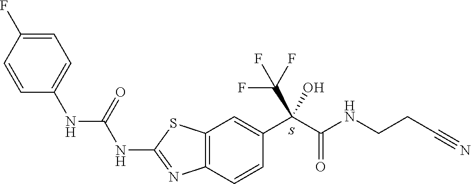 |
| 361 | 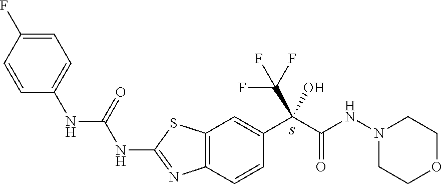 |
| 362 | 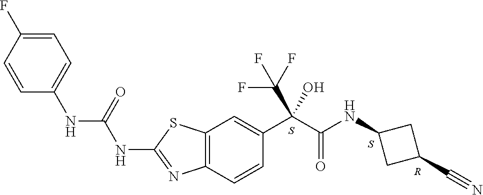 |
| 363 | 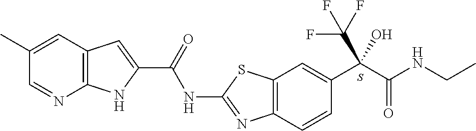 |
| 364 | 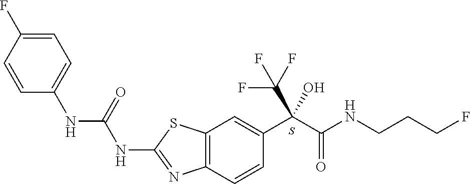 |
| 365 | 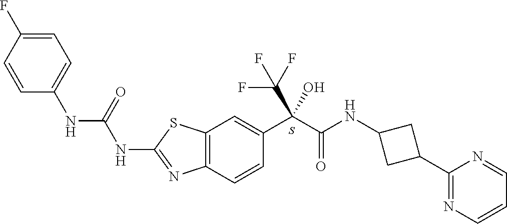 |
| 366 | 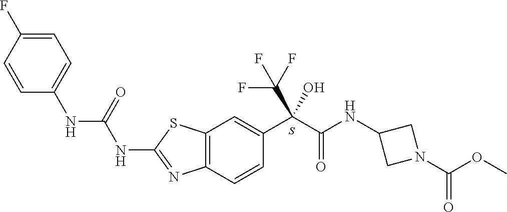 |
| 367 | 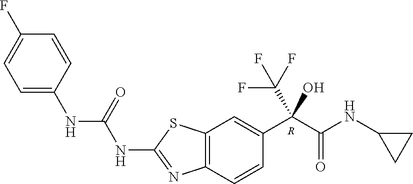 |
| 368 | 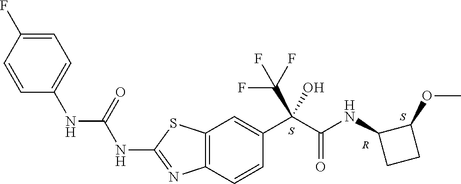 |
| 369 | 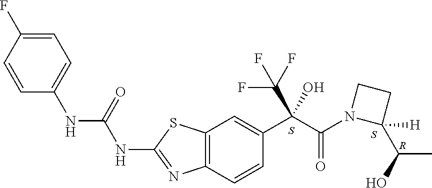 |
| 370 | 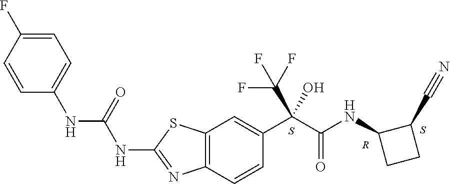 |
| 371 | 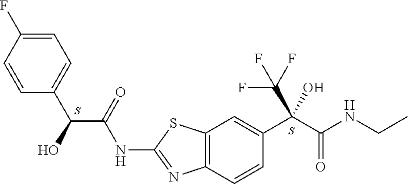 |
| 372 | 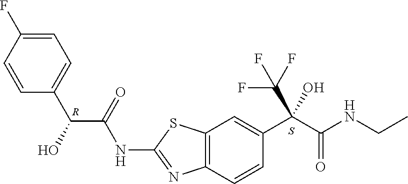 |
| 373 | 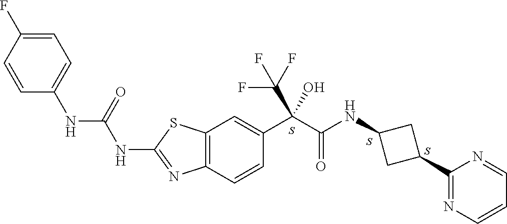 |
| 374 | 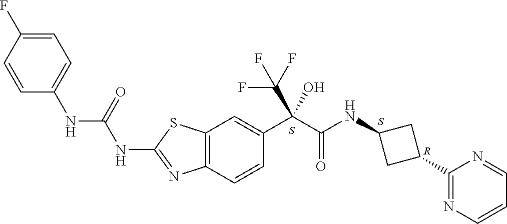 |
| 375 | 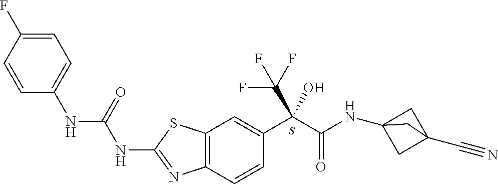 |
| 376 | 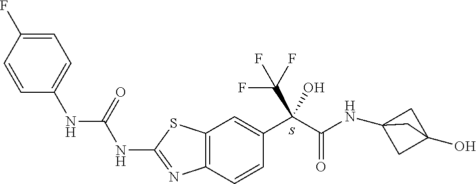 |
| 377 | 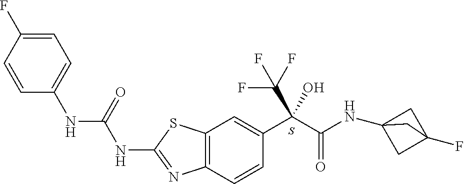 |
| 378 | 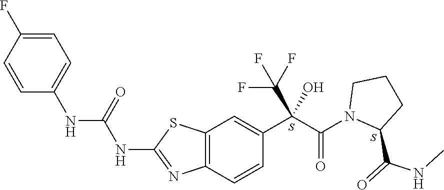 |
| 379 | 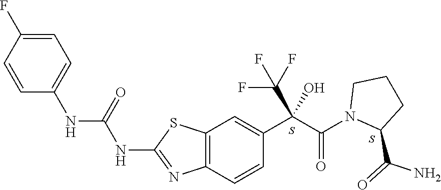 |
| 380 | 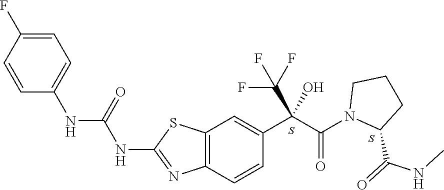 |
| 381 | 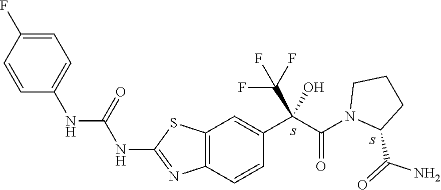 |
| 382 | 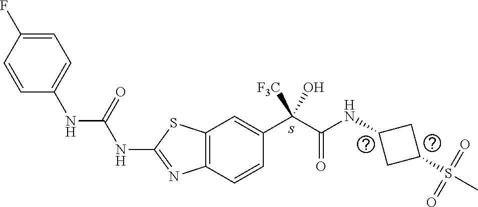 |
| 383 | 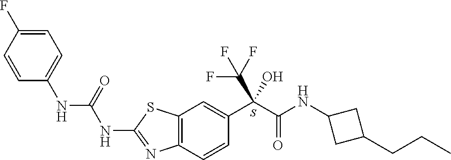 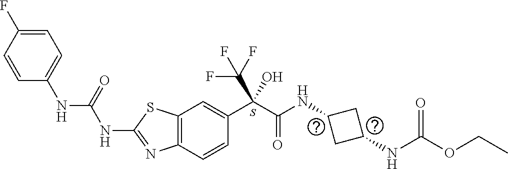 |
| 384 | 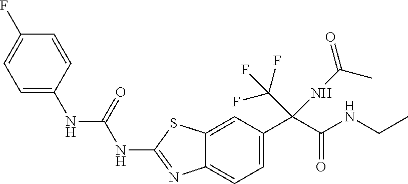 [SMILES: CCNC(=O)C(NC(C)=O)(c1ccc2nc(NC(=O)Nc3ccc(F)cc3)sc2c1)C(F)(F)F] |
| 385 | 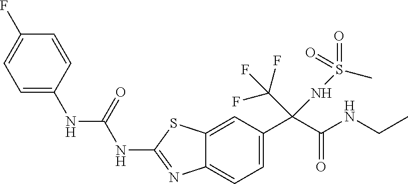 [SMILES: CCNC(=O)C(NS(C)(=O)=O)(c1ccc2nc(NC(=O)Nc3ccc(F)cc3)sc2c1)C(F)(F)F] |
| 386 | 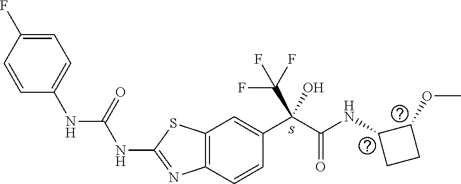 |
| 387 | 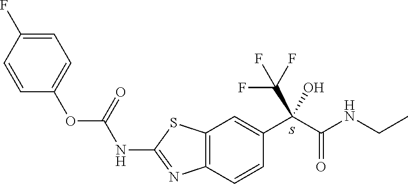 |
| 388 | 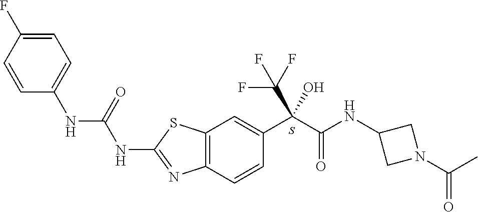 |
| 389 | 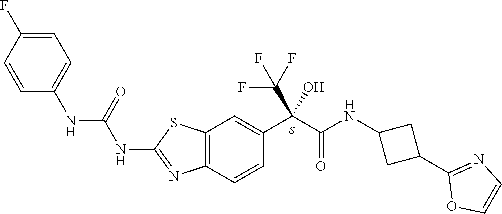 |
| 390 | 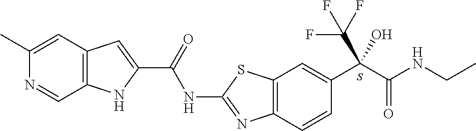 |
| 391 | 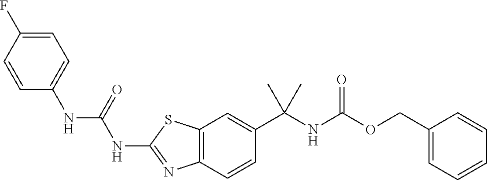 [SMILES: CC(C)(NC(=O)OCc1ccccc1)c1ccc2nc(NC(=O)Nc3ccc(F)cc3)sc2c1] |
| 392 | 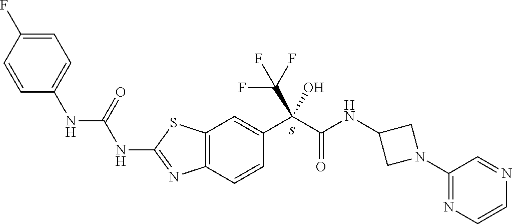 |
| 393 | 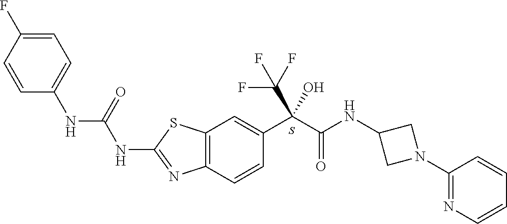 |
| 394 | 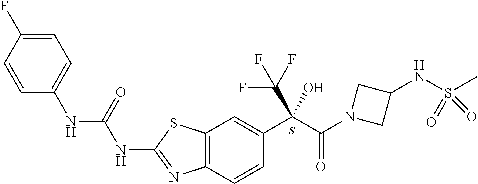 |
| 395 | 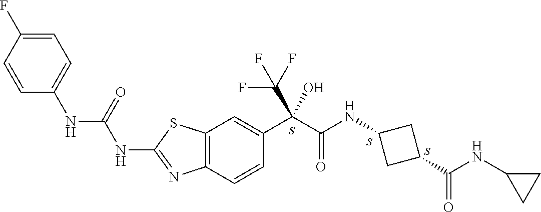 |
| 396 | 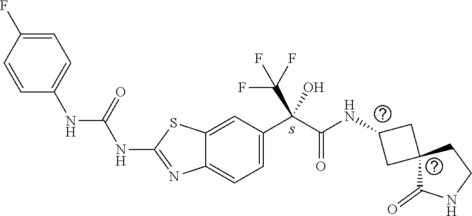 |
| 397 | 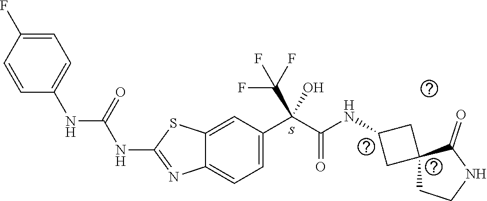 |
| 398 | 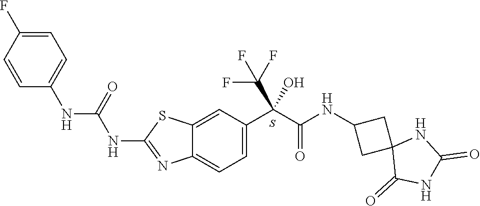 |
| 399 | 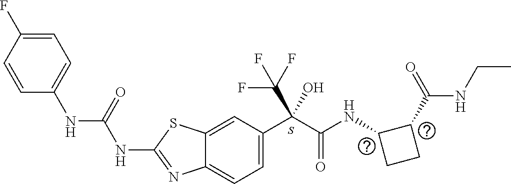 |
| 400 | 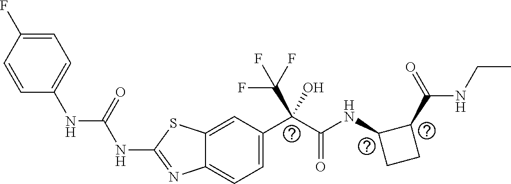 [SMILES: CCNC(=O)[C@H]1CC[C@H]1NC(=O)[C@@](O)(c1ccc2nc(NC(=O)Nc3ccc(F)cc3)sc2c1)C(F)(F)F] |
| 401 | 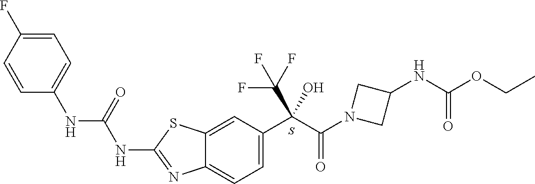 |
| 402 | 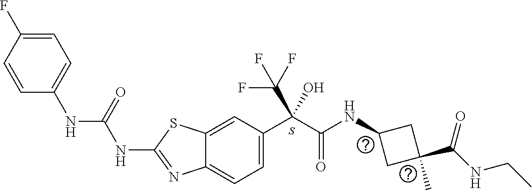 |
| 403 | 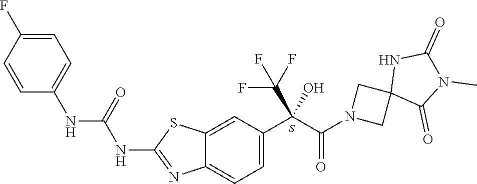 |
| 404 | 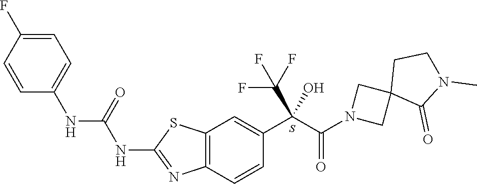 |
| 405 | 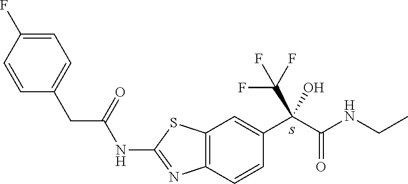 |
| 406 | 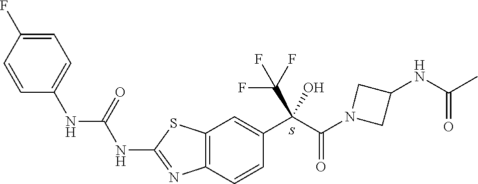 |
| 407 | 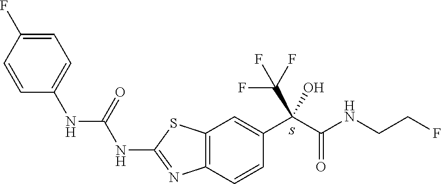 |
| 408 | 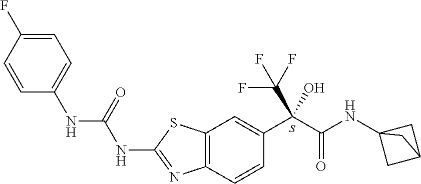 |
| 409 | 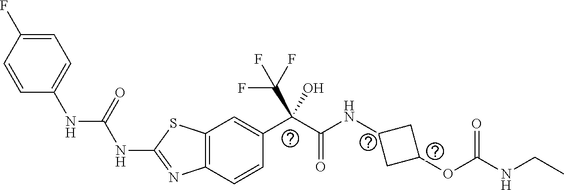 [SMILES: CCNC(=O)OC1CC(NC(=O)[C@@](O)(c2ccc3nc(NC(=O)Nc4ccc(F)cc4)sc3c2)C(F)(F)F)C1] |
| 410 | 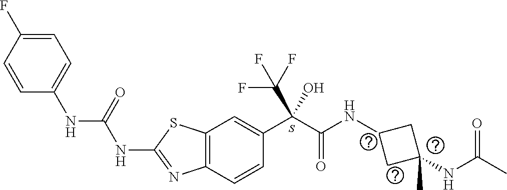 |
| 411 | 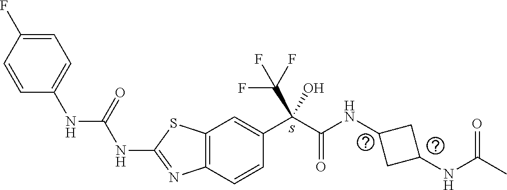 |
| 412 | 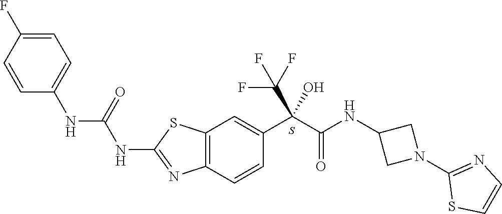 |
| 413 | 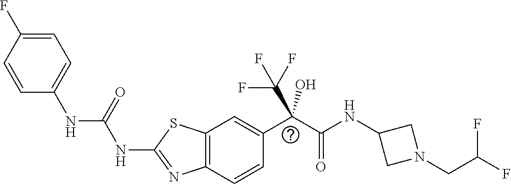 [SMILES: O=C(Nc1ccc(F)cc1)Nc1nc2ccc([C@](O)(C(=O)NC3CN(CC(F)F)C3)C(F)(F)F)cc2s1] |
| 414 | 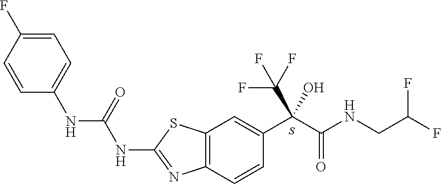 |
| 415 | 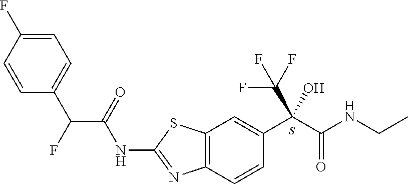 |
| 416 | 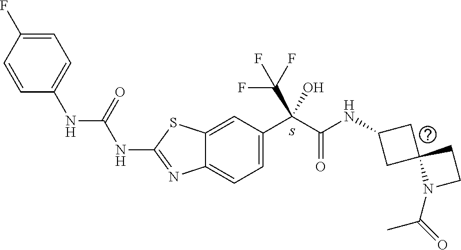 |
| 417 | 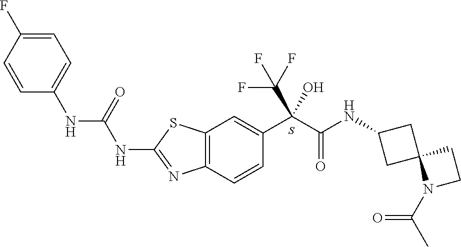 |
| 418 | 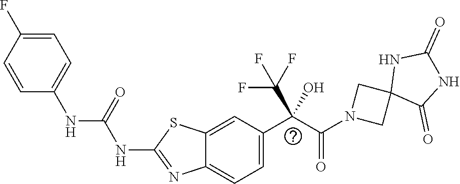 [SMILES: O=C(Nc1ccc(F)cc1)Nc1nc2ccc([C@](O)(C(=O)N3CC4(C3)NC(=O)NC4=O)C(F)(F)F)cc2s1] |
| 419 | 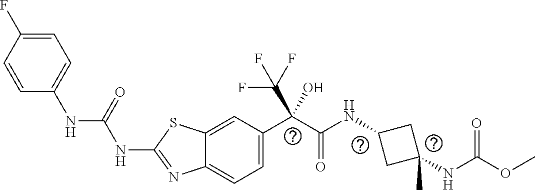 [SMILES: COC(=O)N[C@]1(C)C[C@H](NC(=O)[C@@](O)(c2ccc3nc(NC(=O)Nc4ccc(F)cc4)sc3c2)C(F)(F)F)C1] |
| 420 | 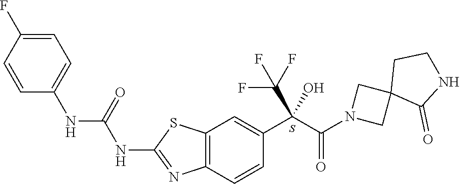 |
| 421 | 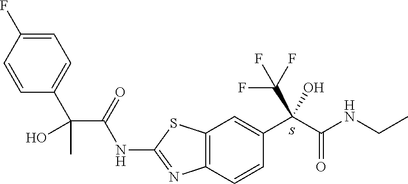 |
| 422 | 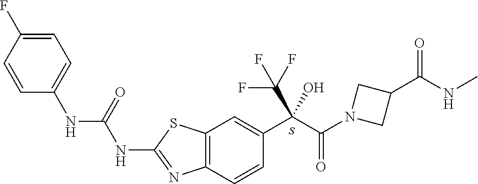 |
| 423 | 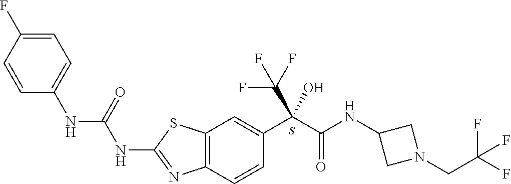 |
| 424 | 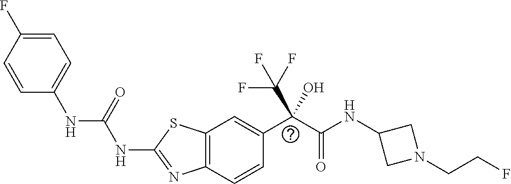 [SMILES: O=C(Nc1ccc(F)cc1)Nc1nc2ccc([C@](O)(C(=O)NC3CN(CCF)C3)C(F)(F)F)cc2s1] |
| 425 | 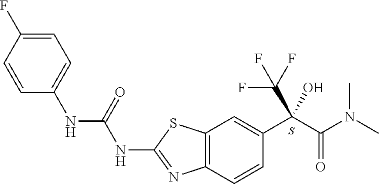 |
| 426 | 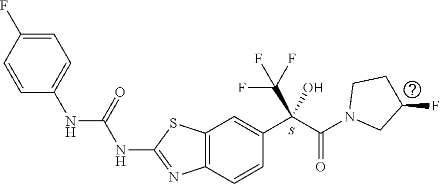 |
| 427 | 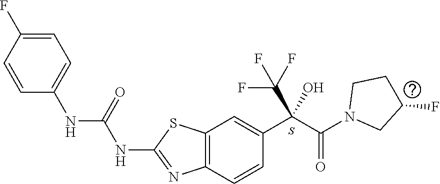 |
| 428 | 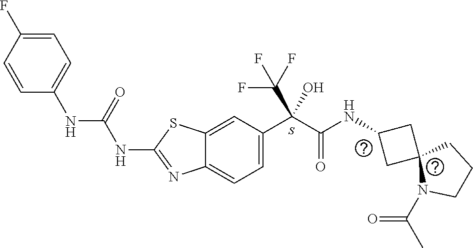 |
| 429 | 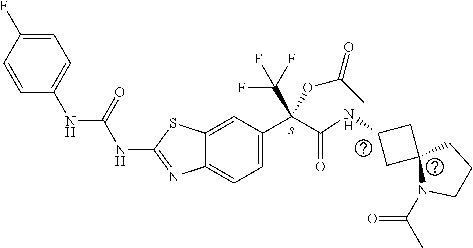 |
| 430 | 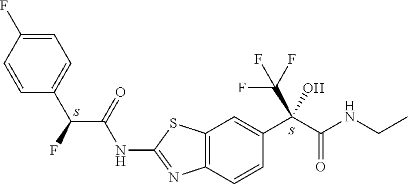 |
| 431 | 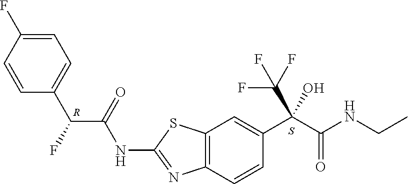 |
| 432 | 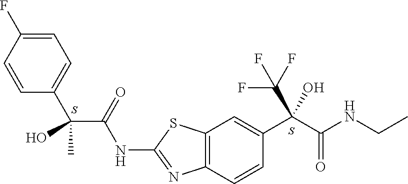 |
| 433 | 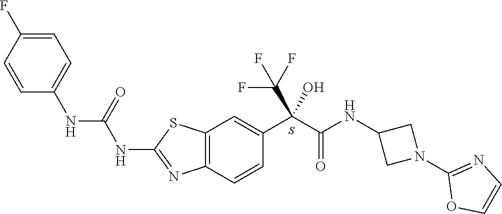 |
| 434 | 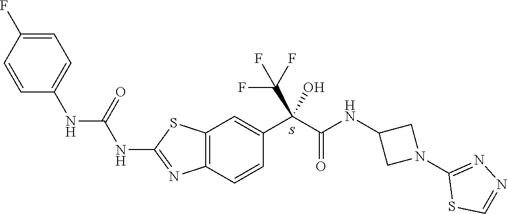 |
| 435 | 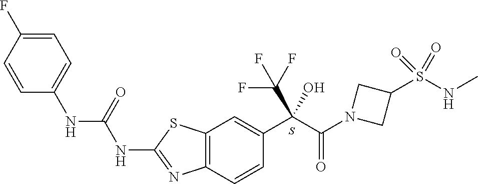 |
| 436 | 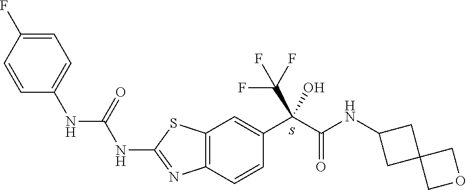 |
| 437 | 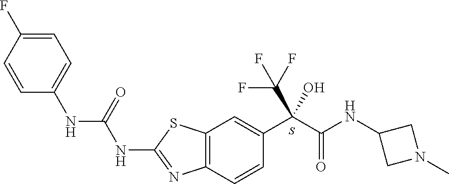 |
| 438 | 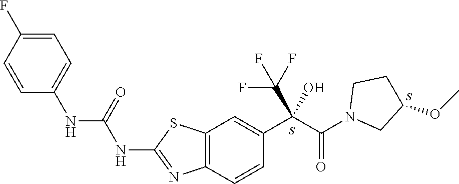 |
| 439 | 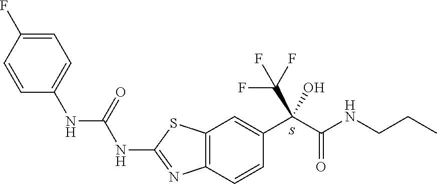 |
| 440 | 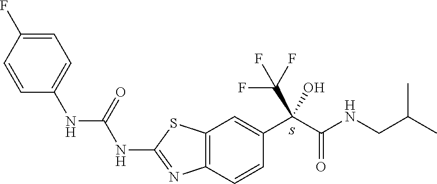 |
| 441 | 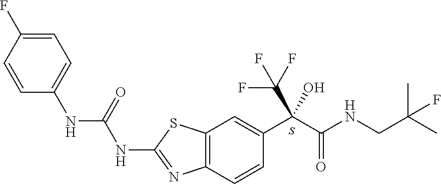 |
| 442 | 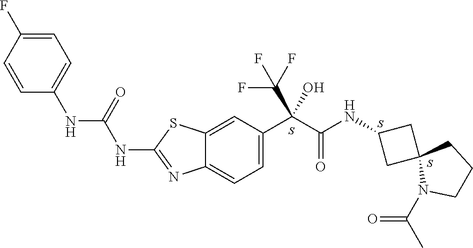 |
| 443 | 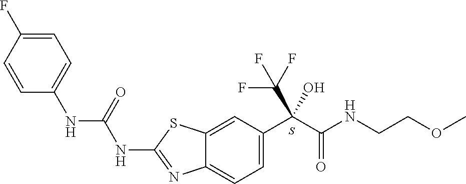 |
| 444 | 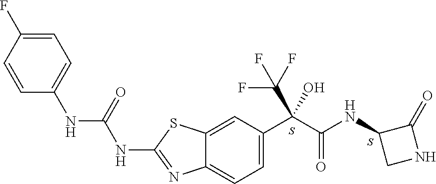 |
| 445 | 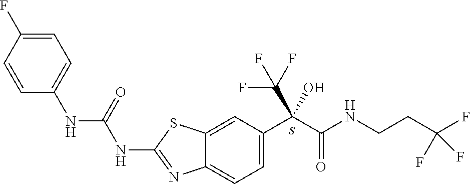 |
| 446 | 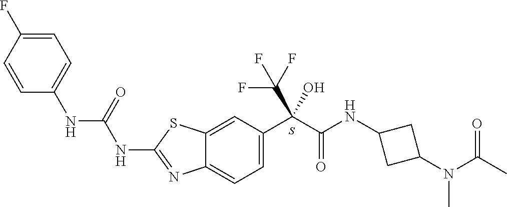 |
| 447 | 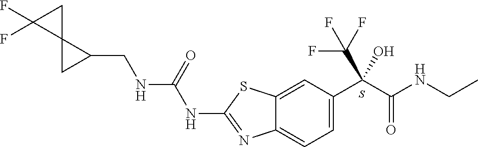 |
| 448 | 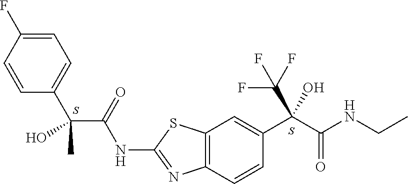 |
| 449 | 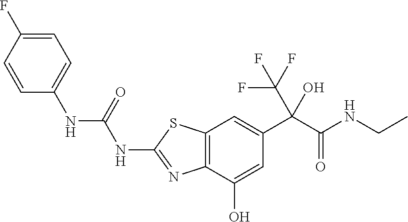 [SMILES: CCNC(=O)C(O)(c1cc(O)c2nc(NC(=O)Nc3ccc(F)cc3)sc2c1)C(F)(F)F] |
| 450 | 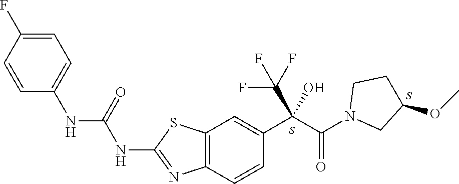 |
| 451 | 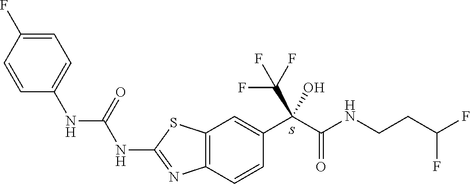 |
| 452 | 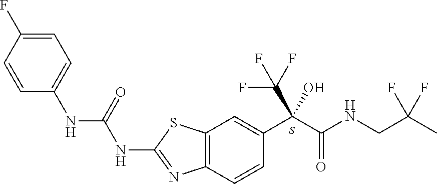 |
| 453 | 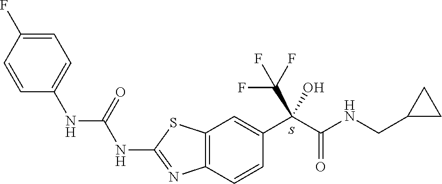 |
| 454 | 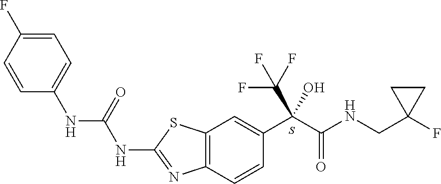 |
| 455 | 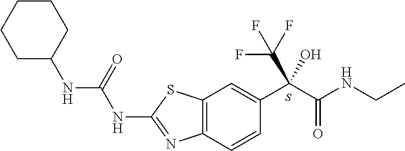 |
| 456 | 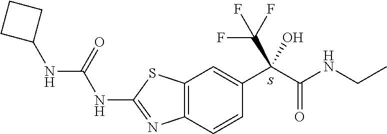 |
| 457 | 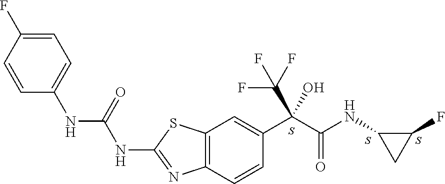 |
| 458 | 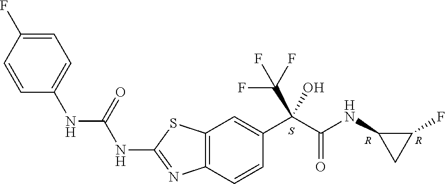 |
| 459 | 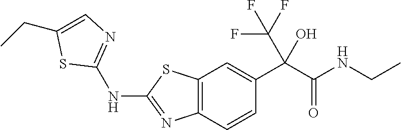 [SMILES: CCNC(=O)C(O)(c1ccc2nc(Nc3ncc(CC)s3)sc2c1)C(F)(F)F] |
| 460 | 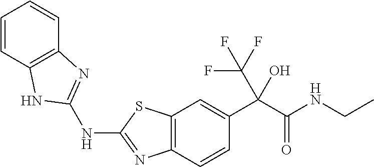 [SMILES: CCNC(=O)C(O)(c1ccc2nc(Nc3nc4ccccc4[nH]3)sc2c1)C(F)(F)F] |
| 461 | 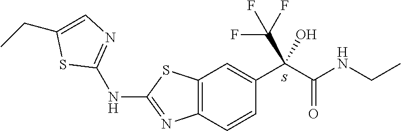 |
| 462 | 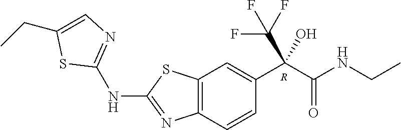 |
| 463 | 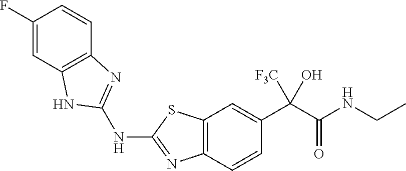 [SMILES: CCNC(=O)C(O)(c1ccc2nc(Nc3nc4ccc(F)cc4[nH]3)sc2c1)C(F)(F)F] |
| 464 | 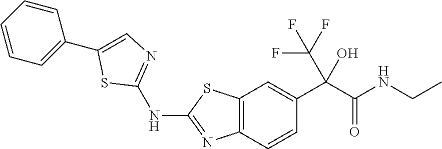 [SMILES: CCNC(=O)C(O)(c1ccc2nc(Nc3ncc(-c4ccccc4)s3)sc2c1)C(F)(F)F] |
| 465 | 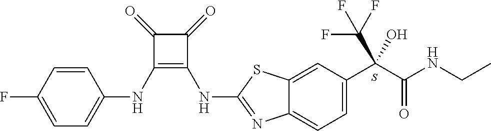 |
| 466 | 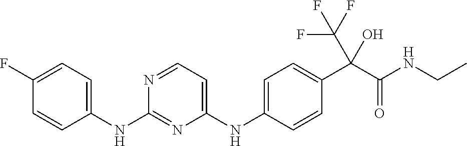 [SMILES: CCNC(=O)C(O)(c1ccc(Nc2ccnc(Nc3ccc(F)cc3)n2)cc1)C(F)(F)F] |
| 467 | 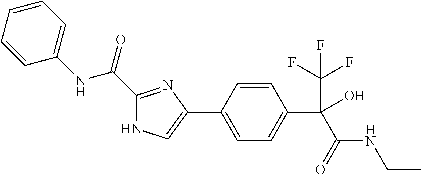 [SMILES: CCNC(=O)C(O)(c1ccc(-c2c[nH]c(C(=O)Nc3ccccc3)n2)cc1)C(F)(F)F] |
| 468 | 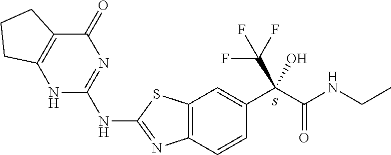 |
| 469 | 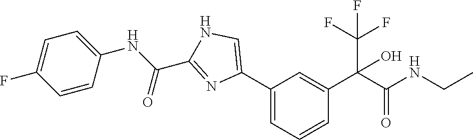 [SMILES: CCNC(=O)C(O)(c1cccc(-c2c[nH]c(C(=O)Nc3ccc(F)cc3)n2)c1)C(F)(F)F] |
| 470 | 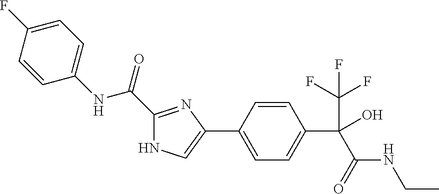 [SMILES: CCNC(=O)C(O)(c1ccc(-c2c[nH]c(C(=O)Nc3ccc(F)cc3)n2)cc1)C(F)(F)F] |
| 471 | 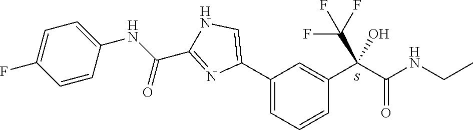 |
| 472 | 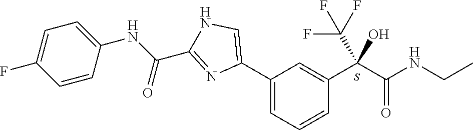 |
| 473 | 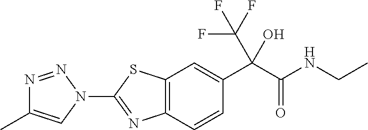 [SMILES: CCNC(=O)C(O)(c1ccc2nc(-n3cc(C)nn3)sc2c1)C(F)(F)F] |
| 474 | 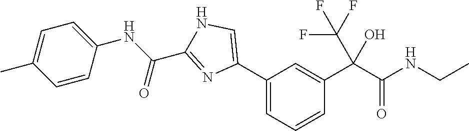 [SMILES: CCNC(=O)C(O)(c1cccc(-c2c[nH]c(C(=O)Nc3ccc(C)cc3)n2)c1)C(F)(F)F] |
| 475 | 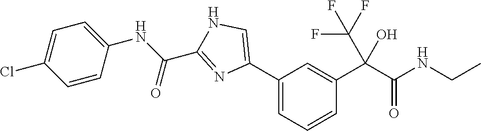 [SMILES: CCNC(=O)C(O)(c1cccc(-c2c[nH]c(C(=O)Nc3ccc(Cl)cc3)n2)c1)C(F)(F)F] |
| 476 | 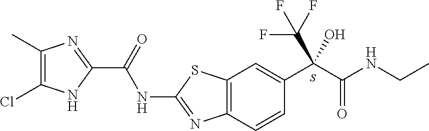 |
| 477 | 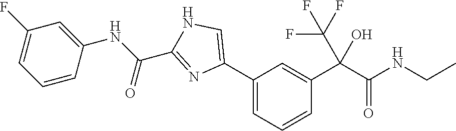 [SMILES: CCNC(=O)C(O)(c1cccc(-c2c[nH]c(C(=O)Nc3cccc(F)c3)n2)c1)C(F)(F)F] |
| 478 | 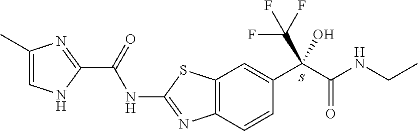 |
| 479 | 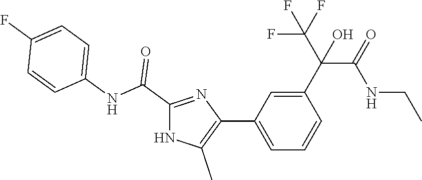 [SMILES: CCNC(=O)C(O)(c1cccc(-c2nc(C(=O)Nc3ccc(F)cc3)[nH]c2C)c1)C(F)(F)F] |
| 480 | 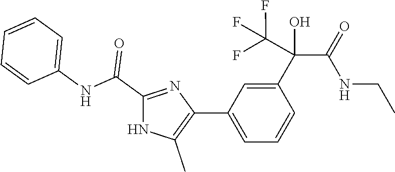 [SMILES: CCNC(=O)C(O)(c1cccc(-c2nc(C(=O)Nc3ccccc3)[nH]c2C)c1)C(F)(F)F] |
| 481 | 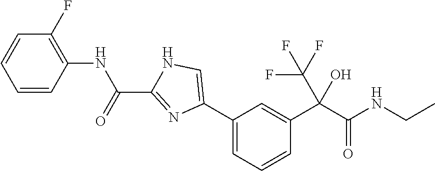 [SMILES: CCNC(=O)C(O)(c1cccc(-c2c[nH]c(C(=O)Nc3ccccc3F)n2)c1)C(F)(F)F] |
| 482 | 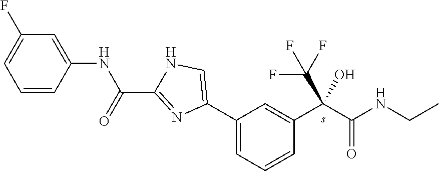 [SMILES: CCNC(=O)[C@@](O)(c1cccc(-c2c[nH]c(C(=O)Nc3cccc(F)c3)n2)c1)C(F)(F)F] |
| 483 | 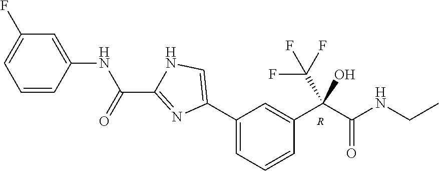 |
| 484 | 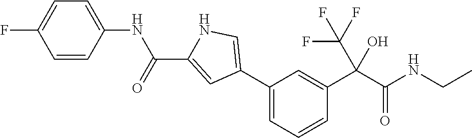 [SMILES: CCNC(=O)C(O)(c1cccc(-c2c[nH]c(C(=O)Nc3ccc(F)cc3)c2)c1)C(F)(F)F] |
| 485 | 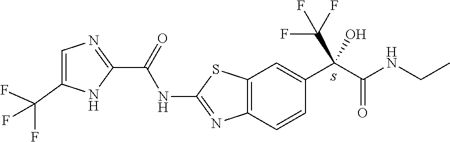 |
| 486 | 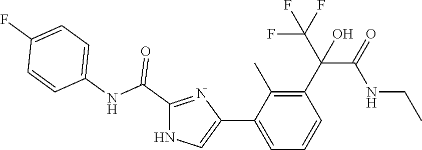 [SMILES: CCNC(=O)C(O)(c1cccc(-c2c[nH]c(C(=O)Nc3ccc(F)cc3)n2)c1C)C(F)(F)F] |
| 487 | 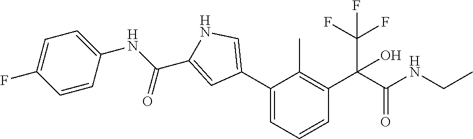 [SMILES: CCNC(=O)C(O)(c1cccc(-c2c[nH]c(C(=O)Nc3ccc(F)cc3)c2)c1C)C(F)(F)F] |
| 488 | 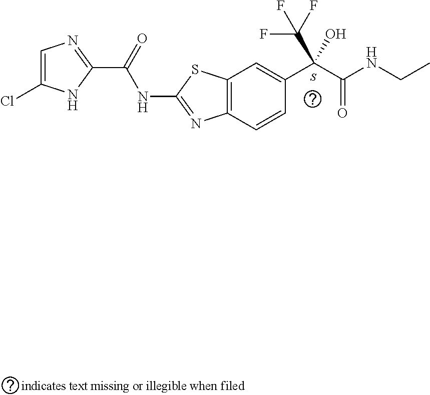 |
| 489 | 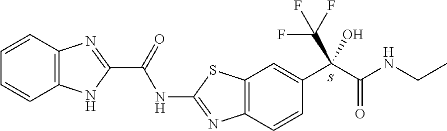 |
| 490 | 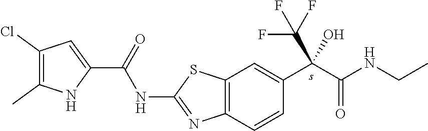 |
| 491 | 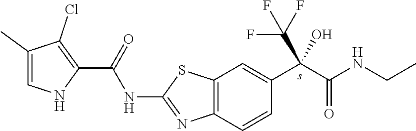 [SMILES: CCNC(=O)[C@@](O)(c1ccc2nc(NC(=O)c3[nH]cc(C)c3Cl)sc2c1)C(F)(F)F] |
| 492 | 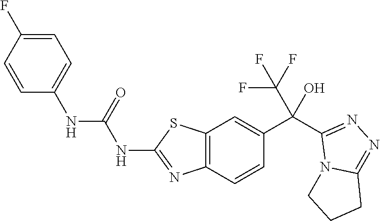 [SMILES: O=C(Nc1ccc(F)cc1)Nc1nc2ccc(C(O)(c3nnc4n3CCC4)C(F)(F)F)cc2s1] |
| 493 | 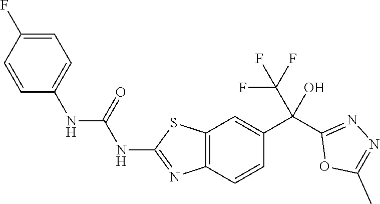 [SMILES: Cc1nnc(C(O)(c2ccc3nc(NC(=O)Nc4ccc(F)cc4)sc3c2)C(F)(F)F)o1] |
| 494 | 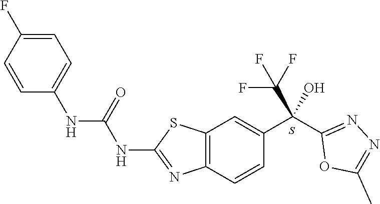 |
| 495 | 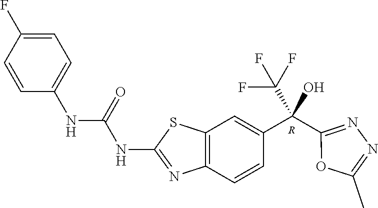 |
| 496 | 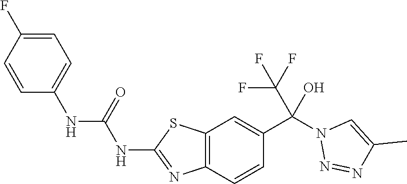 [SMILES: Cc1cn(C(O)(c2ccc3nc(NC(=O)Nc4ccc(F)cc4)sc3c2)C(F)(F)F)nn1] |
| 497 | 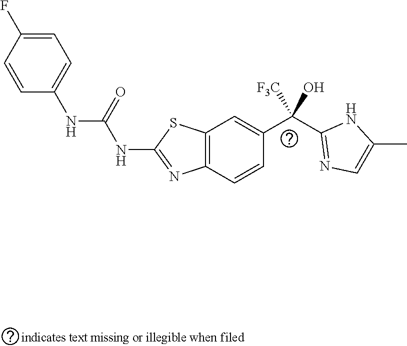 [SMILES: Cc1cnc([C@](O)(c2ccc3nc(NC(=O)Nc4ccc(F)cc4)sc3c2)C(F)(F)F)[nH]1] |
| 498 | 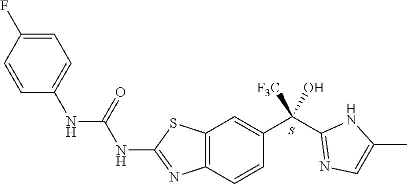 |
| 499 | 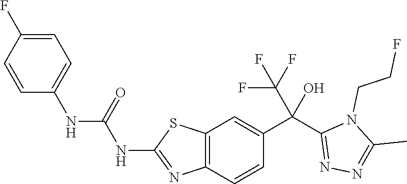 [SMILES: Cc1nnc(C(O)(c2ccc3nc(NC(=O)Nc4ccc(F)cc4)sc3c2)C(F)(F)F)n1CCF] |
| 500 | 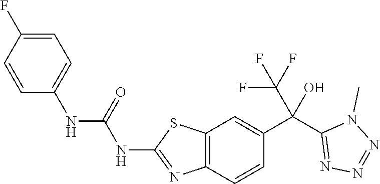 [SMILES: Cn1nnnc1C(O)(c1ccc2nc(NC(=O)Nc3ccc(F)cc3)sc2c1)C(F)(F)F] |
| 501 | 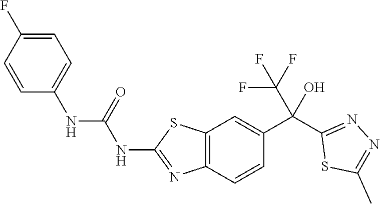 [SMILES: Cc1nnc(C(O)(c2ccc3nc(NC(=O)Nc4ccc(F)cc4)sc3c2)C(F)(F)F)s1] |
| 502 | 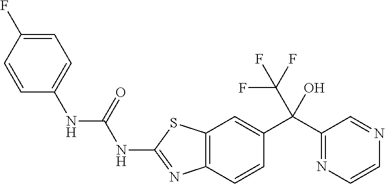 [SMILES: O=C(Nc1ccc(F)cc1)Nc1nc2ccc(C(O)(c3cnccn3)C(F)(F)F)cc2s1] |
| 503 | 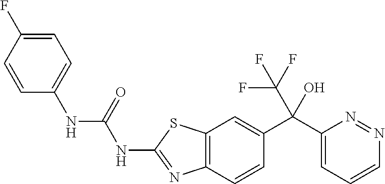 [SMILES: O=C(Nc1ccc(F)cc1)Nc1nc2ccc(C(O)(c3cccnn3)C(F)(F)F)cc2s1] |
| 504 | 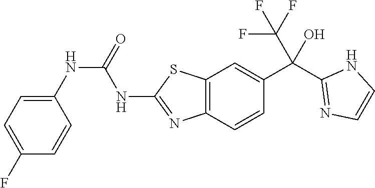 [SMILES: O=C(Nc1ccc(F)cc1)Nc1nc2ccc(C(O)(c3ncc[nH]3)C(F)(F)F)cc2s1] |
| 505 | 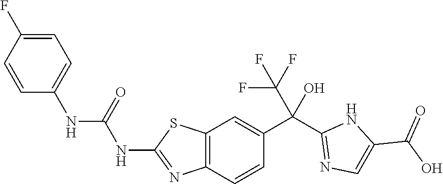 [SMILES: O=C(Nc1ccc(F)cc1)Nc1nc2ccc(C(O)(c3ncc(C(=O)O)[nH]3)C(F)(F)F)cc2s1] |

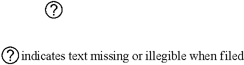

or a pharmaceutically acceptable salt thereof.

120. A pharmaceutical composition, comprising a compound of any one of claims 1 to 119; and at least one pharmaceutically acceptable excipient.

121. A method for treating or preventing osteoporosis, fracture, osteomalacia, arthritis, thrombocytopenia, hypoparathyroidism, hyperphosphatemia or tumoral calcinosis, comprising administering to a subject in need thereof an effective amount of a compound of any one of claims 1 to 119, or a pharmaceutically acceptable salt thereof.
